(19) United States
(12) Patent Application Publication
(10) Pub. No.: US 2023/0081426 A1
(43) Pub. Date: Mar. 16, 2023

(54) COMPOUNDS AND METHODS FOR KRAS MODULATION AND INDICATIONS THEREFOR

(71) Applicant: Plexxikon Inc., South San Francisco, CA (US)

(72) Inventors: Jiazhong Zhang, Foster City, CA (US); John Buell, San Francisco, CA (US); Phuongly Pham, San Francisco, CA (US); Wayne Spevak, Berkeley, CA (US); Mark Vander Wal, Berkeley, CA (US); Xinping Han, Berkeley, CA (US); Chao Zhang, Moraga, CA (US); Zuojun Guo, Pasadena, CA (US); Jack Walleshauser, Berkeley, CA (US); Ying Zhang, Fremont, CA (US); Steven Rank, Union City, CA (US); Yasuyuki Ogawa, Yokosuka-shi (JP); Songyuan Shi, Fremont, CA (US)

(21) Appl. No.: 17/480,111

(22) Filed: Sep. 20, 2021

Related U.S. Application Data
(60) Provisional application No. 63/080,548, filed on Sep. 18, 2020.
(60) Provisional application No. 63/140,248, filed on Jan. 21, 2021.

(51) Int. Cl. A61K 31/517; C07D 487/08; C07D 519/00; A61K 31/5377; A61K 31/5386; C07D 498/08; A61K 31/551

(52) U.S. Cl. CPC A61K 31/517; C07D 487/08; C07D 519/00; A61K 31/551; A61K 31/5386; C07D 498/08; A61K 31/5377

(57) ABSTRACT

Disclosed are compounds of Formula I:

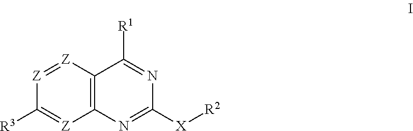

or a pharmaceutically acceptable salt, a solvate, a tautomer, a stereoisomer, or a deuterated analog thereof, wherein: R¹, R², R³, X, and Z are as described in any of the embodiments described in this disclosure; compositions thereof; and uses thereof.

### CROSS-REFERENCE TO RELATED APPLICATIONS

This application claims the benefit under 35 U.S.C. § 119(e) of U.S. Provisional Application No. 63/140,248, filed Jan. 21, 2021, and U.S. Provisional Application No. 63/080,548, filed Sep. 18, 2020, each of which is hereby incorporated by reference in its entirety.

### FIELD

The present invention relates to organic compounds useful for therapy in a mammals, and in particular for modulating KRAS for various diseases associated with the overexpression of KRAS.

### BACKGROUND

The KRAS protein is a GTPase transductor protein encoded by the KRAS proto-oncogene (Kirsten rat sarcoma 2 viral oncogene homolog) that encodes a GTPase transductor protein called KRAS. KRAS belongs to a group of guanosine triphosphate (GTP) binding proteins, known as RAS superfamily or RAS-like GTPases. KRAS protein switches between an inactive to an active form via binding to GTP (active form) and GDP (inactive form). Under physiological conditions, KRAS is predominantly GDP-bound and therefore inactive. The conversion from inactive RAS-GDP to active RAS-GTP further promotes the activation of various signaling pathways, which includes mitogen-activated protein kinase (MAPK) pathway, phosphoinositide 3-kinase (PI3K) pathway and the Ral-GEFs pathway, among them the MAPK pathway is the best characterized. RAS is one of the most frequently mutated oncogenes in human cancer but the frequency and distribution of RAS gene mutations are not uniform, 14. KRAS is the isoform most frequently mutated, which constitutes 86% of RAS mutations (Liu et al, Targeting the untargetable KRAS in cancer, Acta Pharmaceutica Sinica B 2019, 9(5), 871-879).

The extremely high affinity of KRAS for GTP and the abundance of GTP in cell cytoplasm are just some reasons why the development of a KRAS inhibitor drug has been a long and difficult process with no approved drugs to date (Id).

Mutation of glycine 12 (G12) has been associated with causing RAS activation by interfering with GAP binding and GAP-stimulated GTP hydrolysis. (Id). The KRAS-G12C oncoprotein has been recently studied, which is one of the most common KRAS mutants in cancer. The mutant cysteine 12 sits in close proximity to both the nucleotide pocket and the switch regions involved in effector interactions of KRAS protein. Small molecules that form covalent bond with the mutant cysteine demonstrated the possibility of directly and selectively targeting the mutant KRAS-G12C protein. These compounds that covalent bond with the mutant cysteine will be limited to G12C mutant, as the covalent reaction occurs specifically with the thiol group of the cysteine residue (Id). In other examples, cancers have been found to be associated with KRAS G12V mutations (from a glycine to a valine (V) at codon 12) and KRAS G12D mutations, from a glycine to an aspartic acid (D), and such KRAS mediated (Id.)

KRAS initiates the activation of an array of signaling molecules, allowing the transmission of transducing signals that affect a number of essential cellular processes such as cell differentiation, growth, chemotaxis and apoptosis. The activation of KRAS signaling involves GTP/GDP binding, the activation of KRAS signaling is now known as a multi-step process that requires proper KRAS post-translation, plasma membrane-localization and interaction with effector proteins. These mechanistic insights pave the way for the exploration of KRAS signaling targeted therapies.

Recent studies also reported the KRAS G12D-selective inhibitors. KRAS G12D is present, with pancreatic adenocarcinoma, colon adenocarcinoma, lung adenocarcinoma, colorectal adenocarcinoma, and rectal adenocarcinoma having the greatest prevalence (The AACR Project GENIE Consortium, Cancer Discovery. 2017; 7(8):818-83). There are now various therapies with KRAS G12D as a predictive biomarker. KRAS G12D has been an inclusion criterion in various clinical trials for a number of different indications.

KRAS is altered in 80.56% of pancreatic ductal adenocarcinoma patients with KRAS G12D present in 27.78% of all pancreatic ductal adenocarcinoma patients, KRAS G12D has been an inclusion criterion in human clinical trials for pancreatic ductal adenocarcinoma. KRAS is also altered in 44.18% of colorectal carcinoma patients with KRAS G12D present in 13.17% of all colorectal carcinoma patients, and KRAS G12D has been an inclusion criterion in human clinical trials for colorectal carcinoma. KRAS is also altered in 86.36% of pancreatic carcinoma patients with KRAS G12D present in 36.42% of all pancreatic carcinoma patients, and KRAS G12D has been an inclusion criterion in human clinical trials for pancreatic carcinoma. KRAS is also altered in 86.35% of pancreatic adenocarcinoma patients with KRAS G12D present in 36.2% of all pancreatic adenocarcinoma patients, and KRAS G12D has been an inclusion criterion in human clinical trials for pancreatic adenocarcinoma. KRAS is also altered in 44.18% of malignant colorectal neoplasm patients with KRAS G12D present in 13.17% of all malignant colorectal neoplasm patients, and KRAS G12D has been an inclusion criterion in human clinical trials for malignant colorectal neoplasm. KRAS is also altered in 44.21% of rectal carcinoma patients with KRAS G12D present in 11.19% of all rectal carcinoma patients, and KRAS G12D has been an inclusion criterion in human clinical trials for rectal carcinoma. KRAS is also altered in 32.84% of lung adenocarcinoma patients with KRAS G12D present in 4.77% of all lung adenocarcinoma patients, and KRAS G12D is an inclusion criterion in human clinical trials for lung adenocarcinoma. KRAS has been altered in 29.61% of non-small cell lung carcinoma patients with KRAS G12D present in 4.26% of all non-small cell lung carcinoma patients, and KRAS G12D has been an inclusion criteria in human clinical trials for non-small cell lung carcinoma. KRAS is also altered in 44.18% of colorectal carcinoma patients with KRAS G12D present in 13.17% of all colorectal carcinoma patients, and KRAS G12D has been an inclusion criterion in human clinical trials in human clinical trials for colorectal carcinoma. KRAS is also altered in 17.89% of malignant solid tumor patients with KRAS G12D present in 4.73% of all malignant solid tumor patients, and KRAS G12D has been an inclusion criterion in human clinical trials for malignant solid tumor. KRAS is also altered in 4.45% of acute myeloid leukemia patients with KRAS G12D present in 1.02% of all acute myeloid leukemia patients, and KRAS G12D has been an inclusion criterion in human clinical trials in human clinical trials for acute myeloid leukemia. KRAS is also altered in 4.45% of squamous cell lung carcinoma patients with KRAS G12D present in 0.31% of all squamous cell lung carcinoma patients, and KRAS G12D has been an inclusion criterion in human clinical trials for squamous cell lung carcinoma. KRAS is also altered in 4.1% of small cell lung carcinoma patients with KRAS G12D present in 1.12% of all small cell lung carcinoma patients, and KRAS G12D has been an inclusion criterion in human clinical trials for small cell lung carcinoma. KRAS is also altered in 2.0% of glioma patients with KRAS G12D present in 0.2% of all glioma patients, and KRAS G12D has been an inclusion criterion in human clinical trials for glioma. KRAS is also altered in 1.54% of myelodysplastic syndrome patients with KRAS G12D present in 0.16% of all myelodysplastic syndrome patients, and KRAS G12D has been an inclusion criterion in human clinical trials for myelodysplastic syndrome. KRAS is also altered in 1.99% of breast cancer patients with KRAS G12D present in 0.13% of all breast cancer patients, and KRAS G12D has been an inclusion criterion in human clinical trials for breast cancer. KRAS is also altered in 13.46% of gastric carcinoma patients with KRAS G12D present in 3.72% of all gastric carcinoma patients, and KRAS G12D has been an inclusion criterion in human clinical trials for gastric carcinoma. KRAS is also altered in 10.16% of ovarian carcinoma patients with KRAS G12D present in 2.94% of all ovarian carcinoma patients, and KRAS G12D has been an inclusion criterion in human clinical trials for ovarian carcinoma. KRAS is also altered in 12.96% of multiple myeloma patients with KRAS G12D present in 1.21% of all multiple myeloma patients, and KRAS G12D has been an inclusion criterion in human clinical trials for multiple myeloma. KRAS is also altered in 0.93% of hepatocellular carcinoma patients with KRAS G12D present in 0.46% of all hepatocellular carcinoma patients, and KRAS G12D has been an inclusion criterion in human clinical trials for hepatocellular carcinoma. KRAS is also altered in 2.52% of head and neck squamous cell carcinoma patients with KRAS G12D present in 0.31% of all head and neck squamous cell carcinoma patients, and KRAS G12D has been an inclusion criterion in human clinical trials for head and neck squamous cell carcinoma. KRAS is also altered in 1.6% of glioblastoma patients with KRAS G12D present in 0.14% of all glioblastoma patients, and KRAS G12D has been an inclusion criterion in human clinical trials for glioblastoma. KRAS is also altered in 2.11% of thyroid gland undifferentiated carcinoma patients, and KRAS G12D has been an inclusion criterion in human clinical trials for thyroid gland undifferentiated carcinoma patients.

Recent studies also reported the KRAS G12V selective inhibitors. KRAS G12V is present, pancreatic adenocarcinoma, lung adenocarcinoma, colon adenocarcinoma, colorectal adenocarcinoma, and rectal adenocarcinoma having the greatest prevalence (The AACR Project GENIE Consortium, Cancer Discovery. 2017; 7(8):818-83). There are now various therapies with KRAS G12V as a predictive biomarker. KRAS G12V has been an inclusion criterion in various clinical trials for a number of different indications.

KRAS is altered in 80.56% of pancreatic ductal adenocarcinoma patients with KRAS G12V present in 25.0% of all pancreatic ductal adenocarcinoma patients, KRAS G12V has been an inclusion criterion in human clinical trials for pancreatic ductal adenocarcinoma. KRAS is also altered in 44.18% of colorectal carcinoma patients with KRAS G12V present in 9.13% of all colorectal adenocarcinoma patients, and KRAS G12V has been an inclusion criterion in human clinical trials for colorectal carcinoma. KRAS is also altered in 86.36% of pancreatic carcinoma patients with KRAS G12V present in 27.25% of all pancreatic carcinoma patients, and KRAS G12D has been an inclusion criterion in human clinical trials for pancreatic carcinoma. KRAS is also altered in 86.35% of pancreatic adenocarcinoma patients with KRAS G12V present in 27.33% of all pancreatic adenocarcinoma patients, and KRAS G12V has been an inclusion criterion in human clinical trials for pancreatic adenocarcinoma. KRAS is also altered in 44.18% of malignant colorectal neoplasm patients with KRAS G12V present in 9.13% of all malignant colorectal neoplasm patients, and KRAS G12V has been an inclusion criterion in human clinical trials for malignant colorectal neoplasm. KRAS is also altered in 32.84% of lung adenocarcinoma patients with KRAS G12V present in 6.18% of all lung adenocarcinoma patients, and KRAS G12D is an inclusion criterion in human clinical trials for lung adenocarcinoma. KRAS has been altered in 29.61% of non-small cell lung carcinoma patients with KRAS G12V present in 5.45% of all non-small cell lung carcinoma patients, and KRAS G12V has been an inclusion criteria in human clinical trials for non-small cell lung carcinoma. KRAS is also altered in 44.18% of colorectal carcinoma patients with KRAS G12V present in 9.13% of all colorectal carcinoma patients, and KRAS G12V has been an inclusion criterion in human clinical trials in human clinical trials for colorectal carcinoma. KRAS is also altered in 17.89% of malignant solid tumor patients with KRAS G12V present in 3.92% of all malignant solid tumor patients, and KRAS G12V has been an inclusion criterion in human clinical trials for malignant solid tumor. KRAS is also altered in 4.45% of acute myeloid leukemia patients with KRAS G12V present in 0.6% of all acute myeloid leukemia patients, and KRAS G12V has been an inclusion criterion in human clinical trials in human clinical trials for acute myeloid leukemia. KRAS is also altered in 4.45% of squamous cell lung carcinoma patients with KRAS G12V present in 0.31% of all squamous cell lung carcinoma patients, and KRAS G12V has been an inclusion criterion in human clinical trials for squamous cell lung carcinoma. KRAS is also altered in 4.1% of small cell lung carcinoma patients with KRAS G12V present in 0.75% of all small cell lung carcinoma patients, and KRAS G12V has been an inclusion criterion in human clinical trials for small cell lung carcinoma. KRAS is also altered in 2.0% of glioma patients with KRAS G12V present in 0.15% of all glioma patients, and KRAS G12V has been an inclusion criterion in human clinical trials for glioma. KRAS is also altered in 1.54% of myelodysplastic syndrome patients with KRAS G12V present in 0.16% of all myelodysplastic syndrome patients, and KRAS G12V has been an inclusion criterion in human clinical trials for myelodysplastic syndrome. KRAS is also altered in 12.96% of multiple myeloma patients with KRAS G12V present in 1.62% of all multiple myeloma patients, and KRAS G12V has been an inclusion criterion in human clinical trials for multiple myeloma.

Compounds that can inhibit KRAS G12C, G12D, or G12V therefore, represent a new class of potential therapeutics capable of modulating tumor growth. As there are no KRAS G12 inhibitors, and more specifically no KRAS G12C, G12D, or G12V inhibitors, that are currently approved for the treatment or prevention of diseases in humans, there is an unmet need for new compounds that are capable of modulating KRAS G12C, G12D, or G12V.

### SUMMARY

One embodiment of the disclosure relates to novel compounds, as described in any of the embodiments herein, or a pharmaceutically acceptable salt, a solvate, a tautomer, a stereoisomer or a deuterated analog thereof, wherein these novel compounds can modulate KRAS. In one embodiment, the compounds inhibit one or more of KRAS G12C, G12D, and G12V. In one embodiment, the compounds of this disclosure inhibit KRAS G12D.

Another embodiment of this disclosure relates to a compound of Formula I:

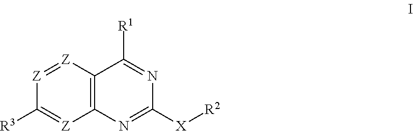

or a pharmaceutically acceptable salt, a solvate, a tautomer, a stereoisomer, or a deuterated analog thereof, wherein: R¹, R², R³, X, and Z are as described in any of the embodiments (including any of the subembodiments thereof) in this disclosure.

Other embodiments and sub-embodiments of Formula I are further described herein in this disclosure.

Another embodiment of the disclosure relates to a pharmaceutical composition comprising a compound according to Formula I or any embodiment and sub-embodiment of Formula I described herein in this disclosure, or a pharmaceutically acceptable salt, a solvate, a tautomer, a stereoisomer or a deuterated analog of any of these compounds, and a pharmaceutically acceptable carrier or excipient.

Another embodiment of the disclosure relates to a pharmaceutical composition comprising a compound according to Formula I, or any embodiment of Formula I described herein in this disclosure, or a pharmaceutically acceptable salt, a solvate, a tautomer, a stereoisomer or a deuterated analog of any of these compounds, and another therapeutic agent.

Another embodiment of this disclosure relates to a method for treating a subject with a disease or condition mediated by KRAS, said method comprising administering to the subject an effective amount of a compound according to Formula I, or any embodiment of Formula I described in this disclosure, or a pharmaceutically acceptable salt, a solvate, a tautomer, a stereoisomer or a deuterated analog of any of these compounds, or a pharmaceutical composition of any of the compounds as described in this disclosure, wherein the disease or condition express aberrantly or otherwise KRAS, or activating mutations or translocations of any of the foregoing. In one embodiment, the KRAS that is modulated is one or more of KRAS G12C, G12D, and G12V. In a more specific embodiment, the KRAS that is modulated is KRAS G12D.

Additional embodiments are described are further described in the Detailed Description of this disclosure.

### DETAILED DESCRIPTION

### I. Definitions

As used herein the following definitions apply unless clearly indicated otherwise:

It is noted here that as used herein and the appended claims, the singular forms “a,” “an,” and “the” include plural reference unless the context clearly dictates otherwise.

Unless a point of attachment indicates otherwise, the chemical moieties listed in the definitions of the variables of Formula I of this disclosure, and all the embodiments thereof, are to be read from left to right, wherein the right hand side is directly attached to the parent structure as defined. However, if a point of attachment (e.g., a dash “—”) is shown on the left hand side of the chemical moiety (e.g., -alkyloxy-C₁-C₆alkyl), then the left hand side of this chemical moiety is attached directly to the parent moiety as defined.

It is assumed that when considering generic descriptions of compounds described herein for the purpose of constructing a compound, such construction results in the creation of a stable structure. That is, one of ordinary skill in the art would recognize that, theoretically, some constructs would not normally be considered as stable compounds (that is, sterically practical and/or synthetically feasible). Where substitution of a variable (e.g., CN) would render an unstable compound, the substitution is not included as resulting in an unstable structure. This clarification also applies to any other chemical group within the definition of an optionally substituted variable that would be considered unstable if the chemical group were to be substituted.

“Alkyl,” by itself, or as part of another substituent, means, unless otherwise stated, a straight or branched chain hydrocarbon, having the number of carbon atoms designated (i.e. C₁-C₆ means one to six carbons). Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, arylalkyl, cycloalkylalkyl, heterocycloalkylalkyl, heteroarylalkyl, etc.), when a prefix is not included to indicate the number of carbon atoms in an alkyl portion, the alkyl moiety or portion thereof will have 12 or fewer main chain carbon atoms or 8 or fewer main chain carbon atoms or 6 or fewer main chain carbon atoms. For example, C₁-C₆alkyl refers to a straight or branched hydrocarbon having 1, 2, 3, 4, 5 or 6 carbon atoms and includes, but is not limited to, —CH₃, C₂alkyl, C₃alkyl, C₄alkyl, C₅alkyl, C₆alkyl, C₁-C₂alkyl, C₂alkyl, C₃alkyl, C₁-C₃alkyl, C₁-C₄alkyl, C₁-C₅alkyl, C₁-C₆alkyl, C₂-C₃alkyl, C₂-C₄alkyl, C₂-C₅alkyl, C₂-C₆alkyl, C₃-C₄alkyl, C₃-C₅alkyl, C₃-C₆alkyl, C₄-C₅alkyl, C₄-C₆alkyl, C₅₋C₆ alkyl and C₆alkyl. While it is understood that substitutions are attached at any available atom to produce a stable compound, when optionally substituted alkyl is an R group of a moiety such as —OR (e.g. alkoxy), —SR (e.g. thioalkyl), —NHR (e.g. alkylamino), —C(O)NHR, and the like, substitution of the alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

“Alkylene” by itself or as part of another substituent means a linear or branched saturated divalent hydrocarbon moiety derived from an alkane having the number of carbon atoms indicated in the prefix. For example, (i.e., C₁-C₆ means one to six carbons; C₁-C₆alkylene is meant to include methylene, ethylene, propylene, 2-methylpropylene, pentylene, hexylene and the like). C₁₋₄ alkylene includes methylene —CH₂—, ethylene —CH₂CH₂—, propylene —CH₂CH₂CH₂—, and isopropylene —CH(CH₃)CH₂—, —CH₂CH(CH₃)—, —CH₂—(CH₂)₂CH₂—, —CH₂—CH(CH₃)CH₂—, —CH₂—C(CH₃)₂—CH₂—CH₂CH(CH₃)—. Typically, an alkyl (or alkylene) group will have from 1 to 24 carbon atoms, with those groups having 10 or fewer, 8 or fewer, or 6 or fewer carbon atoms. When a prefix is not included to indicate the number of carbon atoms in an alkylene portion, the alkylene moiety or portion thereof will have 12 or fewer main chain carbon atoms or 8 or fewer main chain carbon atoms, 6 or fewer main chain carbon atoms, or 4 or fewer main chain carbon atoms, or 3 or fewer main chain carbon atoms, or 2 or fewer main chain carbon atoms, or 1 carbon atom.

“Alkenyl” refers to a linear monovalent hydrocarbon radical or a branched monovalent hydrocarbon radical having the number of carbon atoms indicated in the prefix and containing at least one double bond. For example, C₂-C₆ alkenyl is meant to include ethenyl, propenyl, and the like. “C₂-C₆alkenyl C₁-C₆alkylene” is a group —C₁-C₆alkylene-C₂-C₆alkenyl, where alkenyl and alkylene are as defined herein.

The term “alkenylene” refers to a linear divalent hydrocarbon radical or a branched divalent hydrocarbon radical containing at least one double bond and having the number of carbon atoms indicated in the prefix. Examples of such groups include vinyl, 2-propenyl, crotyl, 2-isopentenyl, 2-(butadienyl), 2,4-pentadienyl, 3-(1,4-pentadienyl), and the higher homologs and isomers.

The term “alkynyl” refers to a monoradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds. In some embodiments, alkynyl groups include ethynyl (—C≡CH), propargyl (or propynyl, e.g. —C≡C—CH₃ and the like. When a prefix is not included to indicate the number of carbon atoms in an alkenyl or alkynyl portion, the alkenyl or alkynyl moiety or portion thereof will have 12 or fewer main chain carbon atoms or 8 or fewer main chain carbon atoms, 6 or fewer main chain carbon atoms or 4 or fewer main chain carbon atoms.

The term “alkynylene” refers to a linear divalent hydrocarbon radical or a branched divalent hydrocarbon radical containing at least one triple bond and having the number of carbon atoms indicated in the prefix. Examples of such groups include ethynyl, 1- and 3-propynyl, 3-butynyl, and the higher homologs and isomers.

“Alkoxy” or “alkoxyl” refers to a —O-alkyl group, where alkyl is as defined herein. By way of example, “C₁-C₆alkoxy” refers to a —O—C₁-C₆alkyl group, where alkyl is as defined herein. While it is understood that substitutions on alkoxy are attached at any available atom to produce a stable compound, substitution of alkoxy is such that O, S, or N (except where N is a heteroaryl ring atom), are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom), or to an alkene or alkyne carbon of the other moiety.

The terms “alkoxyalkyl” and “alkoxyalkylene” refer to an alkyl group substituted with an alkoxy group. By way of example, “C₁-C₆alkoxyC₁-C₆alky 1” refers to C₁-C₆alkyl substituted with a C₁-C₆alkoxy where alkyl and alkoxy are as defined herein, while “C₁-C₃alkoxyC₁-C₃alkylene” refers to C₁-C₃alkyl substituted with a C₁-C₃alkoxy where alkylene and alkoxy are as defined herein.

“Amino” or “amine” denotes the group —NH₂.

“Aryl” by itself, or as part of another substituent, unless otherwise stated, refers to a monocyclic, bicyclic or polycyclic polyunsaturated aromatic hydrocarbon radical containing 6 to 14 ring carbon atoms, which can be a single ring or multiple rings (up to three rings) which are fused together or linked covalently. Aryl, however, does not encompass or overlap in any way with heteroaryl defined below. If one or more aryl rings are fused with a heteroaryl ring, the resulting ring system is heteroaryl. Non-limiting examples of unsubstituted aryl groups include phenyl, 1-naphthyl and 2-naphthyl. The term “arylene” refers to a divalent aryl, wherein the aryl is as defined herein.

“5-6 membered aromatic ring” refers to a phenyl ring or a 5-6 membered heteroaryl ring as defined herein. For purposes of this disclosure, bridgehead atoms cannot be two adjacent atoms on any particular ring.

A “bridged ring” or a “bridged compound” is a carbocyclic or heterocyclic compound or moiety having two or more rings containing a bridge of one to four carbon atoms, and optionally one or more heteroatoms (e.g., O, N or S), that connect two bridgehead atoms. In this disclosure, the phrase “bridged carbocyclic or heterocyclic ring” has the same meaning as the phrase “bridged carbocyclic ring or bridged heterocyclic ring.” For purposes of this disclosure, two bridgehead atoms in a bridged ring cannot the same atom on any particular ring. A “bridged heterocyclic ring” or “bridged heterocycloalkyl” refers to a bridged compound having at least one heteroatom. The bridgehead atoms are part of the skeletal framework of the molecule. Bridged rings (or compounds) may be fully carbocyclic (all carbon skeletal atoms). Below is an example of a bridged ring showing each of the bridge and bridgehead atoms.

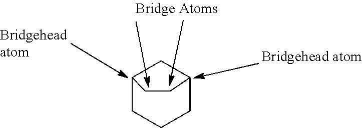

For purposes of this disclosure, a bridged ring is meant to include rings that may optionally have 1-2 C₁-C₃ alkyl groups which are not attached on either its bridge atoms and bridgehead atoms, and these bridged rings can be substituted as described in this disclosure. Other non-limiting examples of bridged rings include bicyclo[1.1.1]pentane, adamantyl, (1s,5s)-bicyclo[3.3.1]nonane, (1R,5S)-6,6-dimethylbicyclo[3.1.1]heptane, (1R,5S)-6,6-dimethylbicyclo[3.1.1]heptane, (1r,2R,4S,5r,6R,8S)-tetracyclo[3.3.1.02,4.06,8]nonane, bicyclo[2.2.1]heptane, bicyclo[2.2.2]octane, and 1-fluorobicyclo[2.2.2]octane.

“Cycloalkyl” or “Carbocycle” or “Carbocyclic” by itself, or as part of another substituent, unless otherwise stated, refers to saturated or partially unsaturated, nonaromatic monocyclic ring, or fused rings, such as bicyclic or tricyclic carbon ring systems, or cubane, having the number of carbon atoms indicated in the prefix or if unspecified having 3-6, also 4-6, and also 5-6 ring members per ring, such as cyclopropyl, cyclopentyl, cyclohexyl, where one or two ring carbon atoms may optionally be replaced by a carbonyl. Further, the term cycloalkyl is intended to encompass ring systems fused to an aromatic ring (e.g., of an aryl), regardless of the point of attachment to the remainder of the molecule. Cycloalkyl refers to hydrocarbon rings having the indicated number of ring atoms (e.g., C₃₋₆ cycloalkyl and 3-6 membered cycloalkyl both mean three to six ring carbon atoms). The term “cycloalkenyl” refers to a cycloalkyl having at least one unit of unsaturation. A substituent of a cycloalkyl or cycloalkenyl may be at the point of attachment of the cycloalkyl or cycloalkenyl group, forming a quaternary center.

“Cycloalkylalkyl” and “cycloalkylalkylene” refer to an -(alkylene)-cycloalkyl group where alkylene as defined herein has the indicated number of carbon atoms or if unspecified having six or fewer carbon atoms; and cycloalkyl is as defined herein has the indicated number of carbon atoms or if unspecified having 3-10, also 3-8, and also 3-6, ring members per ring. By way of example, 4-6 membered cycloalkyl-C₁-C₆alkyl refers to a cycloalkyl with 4-6 carbon atoms attached to an alkylene chain with 1-6 carbon atoms, wherein the alkylene chain is attached to the parent moiety. Other exemplary cycloalkylalkyl groups include, e.g., cyclopropylmethylene, cyclobutylethylene, cyclobutylmethylene, and the like. “Cycloalkylalkynylene” refers to a -(alkynylene)-cycloalkyl group, for example, C₃-C₆cycloalkylC₂-C₆alkynylene is a group —(C₂-C₆alkynylene)-C₃-C₆cycloalkyl. “C₃-C₆cycloalkylethynylene” is a group —C≡C—C₃-C₆cycloalkyl.

The term “cyano” refers to the group —CN. The term “C₁-C₆cyanoalkyl” refers to a C₁-C₆alkyl, as defined herein, that is substituted with 1, 2 or 3 cyano groups. “C₁-C₆cyanoalkylethynylene” is a group —C≡C—C₁-C₆cyanoalkyl. For purposes of clarification, when cyano or CN is included in the definition of a variable (e.g., variable T²), and the variable (e.g., T²) can be substituted, it is understood that CN would not be substituted because a substitution on CN would render an unstable compound.

The term “haloalkyl” refers to an alkyl substituted by one to seven halogen atoms. Haloalkyl includes monohaloalkyl or polyhaloalkyl. For example, the term “C₁-C₆haloalkyl” is meant to include trifluoromethyl, difluoromethyl, 2,2,2-trifluoroethyl, 4-chlorobutyl, 3-bromopropyl, and the like. Further, the term “haloalkylene” refers to an alkylene substituted by one to seven halogen atoms.

The term “haloalkoxy” or “haloalkoxyl” refers to a —O-haloalkyl group, where haloalkyl is as defined herein. Haloalkoxyl includes monohaloalkyloxyl or polyhaloalkoxyl. For example, the term “C₁-C₆haloalkoxyl” is meant to include trifluoromethyloxy, difluoromethyloxy, and the like.

“Halogen” or “halo” refers to all halogens, that is, chloro (Cl), fluoro (F), bromo (Br), or iodo (I).

“Heteroatom” is meant to include oxygen (O), nitrogen (N), and sulfur (S).

“Heteroaryl” refers to a monocyclic or bicyclic aromatic ring radical containing 5-9 ring atoms (also referred to in this disclosure as a 5-9 membered heteroaryl, including monocyclic aromatic ring radicals containing 5 or 6 ring atoms (also referred to in this disclosure as a 5-6 membered heteroaryl), containing one or more, 14, 13, or 12, heteroatoms independently selected from the group consisting of O, S, and N. Any aromatic ring or ring system containing at least one heteroatom is a heteroaryl regardless of the point of attachment (i.e., through any one of the fused rings). Heteroaryl is also intended to include moieties having an oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridyl, pyridazinyl, pyrazinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, triazinyl, quinoxalinyl, cinnolinyl, phthalazinyl, benzotriazinyl, benzimidazolyl, benzopyrazolyl, benzotriazolyl, benzisoxazolyl, isobenzofuryl, isoindolyl, indolizinyl, thienopyridyl, thienopyrimidinyl, pyrazolopyrimidinyl, imidazopyridines, benzothiaxolyl, benzothienyl, quinolyl, isoquinolyl, indazolyl, pteridinyl and thiadiazolyl. “Nitrogen containing heteroaryl” refers to heteroaryl wherein at least one of the ring heteroatoms is N.

“Heterocycloalkyl” refers to a saturated or unsaturated nonaromatic cycloalkyl group that contains from one to five heteroatoms selected from N, O, S (including S(O) and S(O)₂), or P (including phosphine oxide) wherein the nitrogen, sulfur, and phosphorous atoms are optionally oxidized, and the nitrogen atom(s) are optionally quarternized, the remaining ring atoms being C, where one or two C atoms may optionally be present as a carbonyl. Further, the term heterocycloalkyl is intended to encompass any ring or ring system containing at least one heteroatom that is not a heteroaryl, regardless of the point of attachment to the remainder of the molecule. Heterocycloalkyl groups include those having a ring with a formally charge-separated aromatic resonance structure, for example, N-methylpyridonyl. The heterocycloalkyl may include one or two ring carbon atoms present as oxo groups, and can include sulfone and sulfoxide derivatives. The heterocycloalkyl may be a monocyclic, a fused bicyclic or a fused polycyclic ring system of 3 to 12, 4 to 10, 5 to 10, or 5 to 6 ring atoms in which one to five ring atoms are heteroatoms selected from —N═, —N—, —O—, —S—, —S(O)—, or —S(O)₂— and further wherein one or two ring atoms are optionally replaced by a —C(O)— group. As an example, a 4-6 membered heterocycloalkyl is a heterocycloalkyl with 4-6 ring members having at least one heteroatom. The heterocycloalkyl can also be a heterocyclic alkyl ring fused with a cycloalkyl, an aryl or a heteroaryl ring.

Non limiting examples of heterocycloalkyl groups include pyrrolidinyl, piperidinyl, morpholinyl, pyridonyl, indolin-2-one, 3,4-dihydro-2H-benzo[b][1,4]oxazine, 2H-benzo[b][1,4]oxazin-3(4H)-one, and the like. A heterocycloalkyl group can be attached to the remainder of the molecule through a ring carbon or a heteroatom. Unsaturated heterocycloalkyls include, without limitation heterocycloalkenyl, which refers to a heterocycloalkyl having at least one unit of unsaturation. A substituent of a heterocycloalkyl may be at the point of attachment of the heterocycloalkyl group forming a quaternary center.

“Hydroxyl” or “hydroxy” refers to the group OH. The term “hydroxyalkyl” or “hydroxyalkylene” refers to an alkyl group or alkylene group, respectively as defined herein, substituted with 1-5 hydroxy groups.

The term “oxo” refers to C(═O) or (O). In some embodiments, two possible points of attachment on a carbon form an oxo group.

“Optional substituents” or “optionally substituted” as used throughout the disclosure means that the substitution on a compound may or may not occur, and that the description includes instances where the substitution occurs and instances in which the substitution does not. For example, the phrase “optionally substituted with 1-3 T¹ groups” means that the T¹ group may but need not be present. It is assumed in this disclosure that optional substitution on a compound occurs in a way that would result in a stable compound. In another example, if T² is defined in the specification as being substituted with 0-4 R⁸, and a particular T² group can only take 0-1 R⁸ substituents to remain a stable compound, it is understood, under this definition of T², that this particular T² group can only be substituted with 0-1 R⁸ groups, and not beyond the maximum number of R⁸ substituents to render an unstable compound.

“Spiro carbon atom” is a carbon atom which is common to two rings. A “carbocyclic spiro ring” comprises two cycloalkyl rings joined at one common spiro carbon atom as shown in this example:

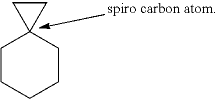

A “heterocyclic spiro ring” comprises a cycloalkyl or heterocycloalkyl ring joined at one common spiro carbon atom to a heterocyclic ring as shown in this example:

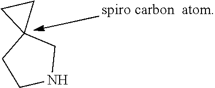

The term “aminoalkyl” refers to -alkylene-NH₂.

The term “alkylamino” refers amino substituted with one or two alkyl, and may be represented as —NH-alkyl or —N-(alkyl)₂. Accordingly, the term “alkylaminoalkyl” refers to alkyl substituted with at least one alkylamino group, and may be represented as, e.g. -alkylene-NH-alkyl or -alkylene-N-(alkyl)₂.

The term “alkylaminocarbonyl” refers to the group —C(O)-aminoalkyl, where aminoalkyl is as define above.

The term “alkylcarbonylaminoalkyl” refers to alkyl-C(O)-aminoalkyl, where alkyl and aminoalkyl are as defined herein.

As used herein in connection with compounds of the disclosure, the term “synthesizing” and like terms means chemical synthesis from one or more precursor materials.

As used herein, the term “composition” refers to a formulation suitable for administration to an intended animal subject for therapeutic purposes that contains at least one pharmaceutically active compound and at least one pharmaceutically acceptable carrier or excipient.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

“Pharmaceutically acceptable salt” refers to a salt which is acceptable for administration to a patient, such as a mammal (e.g., salts having acceptable mammalian safety for a given dosage regime). Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. Such salts can be derived from pharmaceutically acceptable inorganic or organic bases and from pharmaceutically-acceptable inorganic or organic acids, depending on the particular substituents found on the compounds described herein.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

When compounds of the present disclosure contain relatively acidic functionalities, base addition salts can be obtained by contacting the neutral form of such compounds with a sufficient amount of the desired base (i.e. a primary, secondary, tertiary, quaternary, or cyclic amine; an alkali metal hydroxide; alkaline earth metal hydroxide; or the like), either neat or in a suitable inert solvent. The desired acid can be, for example, a pyranosidyl acid (such as glucuronic acid or galacturonic acid), an alpha-hydroxy acid (such as citric acid or tartaric acid), an amino acid (such as aspartic acid or glutamic acid), an aromatic acid (such as benzoic acid or cinnamic acid), a sulfonic acid (such as p-toluenesulfonic acid or ethanesulfonic acid), or the like. In some embodiments, salts can be derived from pharmaceutically acceptable acids such as acetic, trifluoroacetic, propionic, ascorbic, benzenesulfonic, benzoic, camphorsulfonic, citric, ethanesulfonic, fumaric, glycolic, gluconic, glucoronic, glutamic, hippuric, hydrobromic, hydrochloric, isethionic, lactic, lactobionic, maleic, malic, malonic, mandelic, oxalic, methanesulfonic, mucic, naphthalenesulfonic, nicotinic, nitric, pamoic, pantothenic, phosphoric, succinic, sulfuric, sulfamic, hydroiodic, carbonic, tartaric, p-toluenesulfonic, pyruvic, aspartic, benzoic, cinnamic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylsulfamic, cyclohexylaminosulfonic, quinic, algenic, hydroxybutyric, galactaric and galacturonic acid and the like.

Also included are salts of amino acids such as arginate and the like, and salts of organic acids like glucuronic or galactunoric acids and the like (see, for example, Berge, S. M. et al., “Pharmaceutical Salts,” J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The pharmaceutically acceptable salt of the different compounds may be present as a complex. Examples of complexes include 8-chlorotheophylline complex (analogous to, e.g., dimenhydrinate; diphenhydramine 8-chlorotheophylline (1:1) complex; Dramamine) and various cyclodextrin inclusion complexes.

The term “deuterated” as used herein alone or as part of a group, means substituted deuterium atoms. The term “deuterated analog” as used herein alone or as part of a group, means a compound containing substituted deuterium atoms in place of hydrogen atoms. The deuterated analog of the disclosure may be a fully or partially deuterium substituted derivative. In some embodiments, the deuterium substituted derivative of the disclosure holds a fully or partially deuterium substituted alkyl, aryl or heteroaryl group.

The disclosure also embraces isotopically-labeled compounds of the present disclosure which are identical to those recited herein, but for the fact that one or more atoms are replaced by an atom having an atomic mass or mass number different from the atomic mass or mass number usually found in nature. All isotopic variations of the compounds of the present disclosure, whether radioactive or not, are intended to be encompassed within the scope of the present disclosure. Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Unless otherwise stated, when a position is designated specifically as “H” or “hydrogen,” the position is understood to have hydrogen at its natural abundance isotopic composition or its isotopes, such as deuterium (D) or tritium (³H). Certain isotopically-labeled compounds of the present disclosure (e.g., those labeled with ³H and ¹⁴C) are useful in compound and/or substrate tissue distribution assays. Tritiated (i.e., ³H) and carbon-14 (i.e., ¹⁴C) and fluorine-18 (¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those described in the Schemes and in the Examples herein below, by substituting an isotopically labeled reagent for a non-isotopically labeled reagent.

“Prodrugs” means any compound which releases an active parent drug according to Formula I in vivo when such prodrug is administered to a subject. Prodrugs of a compound of Formula I are prepared by modifying functional groups present in the compound of Formula I in such a way, either in routine manipulation or in vivo, that the modifications may be cleaved in vivo to release the parent compound. Prodrugs may proceed from prodrug form to active form in a single step or may have one or more intermediate forms which may themselves have activity or may be inactive. Some prodrugs are activated enzymatically to yield the active compound, or a compound which, upon further chemical reaction, yields the active compound. Prodrugs include compounds of Formula I wherein a hydroxy, amino, carboxyl or sulfhydryl group in a compound of Formula I is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds of Formula I, and the like. Other examples of prodrugs include, without limitation, carbonates, ureides, solvates, or hydrates of the active compound. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

(1) Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

(2) Reductive reactions: Reductive reactions are exemplified without limitation to reactions such as reduction of carbonyl functionalities, reduction of alcohol functionalities and carbon-carbon double bonds, reduction of nitrogen-containing functional groups, and other reduction reactions.

(3) Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng el al., U.S. Patent Publ. No. 2004/0077595, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g. stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols.

The term “carrier” is also meant to include microspheres, liposomes, micelles, nanoparticles (naturally-equipped nanocarriers, for example, exosomes), and the like. It is known that exosomes can be highly effective drug carriers, and there are various ways in which drugs can be loaded into exosomes, including those techniques described in J Control Release. 2015 Dec. 10; 219: 396-405, the contents of which are incorporated by reference in its entirety.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J. Med. Chem., 40:2011-2016; Shan et al., 1997, J Pharm Sci 86(7):756-757; Bagshawe, 1995, Drug Dev. Res., 34:220-230.

“Tautomer” means compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The tautomers also refer to one of two or more structural isomers that exist in equilibrium and are readily converted from one isomeric form to another. Examples of include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) can occur. The compounds described herein may have one or more tautomers and therefore include various isomers. A person of ordinary skill in the art would recognize that other tautomeric ring atom arrangements are possible. All such isomeric forms of these compounds are expressly included in the present disclosure.

“Isomers” mean compounds having identical molecular Formulae but which differ in the nature or sequence of bonding of their atoms or in the arrangement of their atoms in space. Isomers that differ in the arrangement of their atoms in space are termed “stereoisomers.” “Stereoisomer” and “stereoisomers” refer to compounds that exist in different stereoisomeric forms, for example, if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers.” When a compound has an asymmetric center, for example, an atom such as carbon bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.” As another example, stereoisomers include geometric isomers, such as cis- or trans-orientation of substituents on adjacent carbons of a double bond. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6th edition J. March, John Wiley and Sons, New York, 2007) differ in the chirality of one or more stereocenters.

“Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

As used herein, the terms “ligand” and “modulator” are used equivalently to refer to a compound that changes (i.e., increases or decreases) the activity of a target biomolecule, e.g., an enzyme such as those described herein. Generally a ligand or modulator will be a small molecule, where “small molecule refers to a compound with a molecular weight of 1500 Daltons or less, 1000 Daltons or less, 800 Daltons or less, or 600 Daltons or less. Thus, an “improved ligand” is one that possesses better pharmacological and/or pharmacokinetic properties than a reference compound, where “better” can be defined by one skilled in the relevant art for a particular biological system or therapeutic use.

The term “binds” in connection with the interaction between a target and a potential binding compound indicates that the potential binding compound associates with the target to a statistically significant degree as compared to association with proteins generally (i.e., non-specific binding). Thus, the term “binding compound” refers to a compound that has a statistically significant association with a target molecule. In some embodiments, a binding compound interacts with a specified target with a dissociation constant (K_{D}) of 10 mM or less, 1,000 μM or less, 100 μM or less, 10 μM or less, 1 μM or less, 1,000 nM or less, 100 nM or less, 10 nM or less, or 1 nM or less. In the context of compounds binding to a target, the terms “greater affinity” and “selective” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

The terms “modulate,” “modulation,” and the like refer to the ability of a compound to increase or decrease the function and/or expression of a target, such as KRAS, where such function may include transcription regulatory activity and/or binding. Modulation may occur in vitro or in vivo. Modulation, as described herein, includes the inhibition, antagonism, partial antagonism, activation, agonism or partial agonism of a function or characteristic associated with KRAS, either directly or indirectly, and/or the upregulation or downregulation of the expression KRAS, either directly or indirectly. In another embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction.

As used herein, the terms “treat,” “treating,” “therapy,” “therapies,” and like terms refer to the administration of material, e.g., any one or more compound(s) as described herein in an amount effective to prevent, alleviate, or ameliorate one or more symptoms of a disease or condition, i.e., indication, and/or to prolong the survival of the subject being treated.

The terms “prevent,” “preventing,” “prevention” and grammatical variations thereof as used herein, refers to a method of partially or completely delaying or precluding the onset or recurrence of a disease, disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “subject,” “animal subject,” and the like refers to a living organism including, but not limited to, human and non-human vertebrates, e.g. any mammal, such as a human, other primates, sports animals and animals of commercial interest such as cattle, horses, ovines, or porcines, rodents, or pets such as dogs and cats.

“Unit dosage form” refers to a composition intended for a single administration to treat a subject suffering from a disease or medical condition. Each unit dosage form typically comprises a compound of this disclosure plus one or more pharmaceutically acceptable excipients. Examples of unit dosage forms are individual tablets, individual capsules, bulk powders, liquid solutions, ointments, creams, eye drops, suppositories, emulsions or suspensions. Treatment of the disease or condition may require periodic administration of unit dosage forms, for example: one unit dosage form two or more times a day, one with each meal, one every four hours or other interval, or only one per day. The expression “oral unit dosage form” indicates a unit dosage form designed to be taken orally.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In the present context, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

The ability of a compound to inhibit the function of KRAS can be demonstrated in a biochemical assay, e.g., binding assay, or a cell based assay.

As used herein, the term “KRAS” mediated disease or condition” refers to a disease or condition in which the biological function of KRAS (e.g., KRAS G12C and/or KRAS G12D) affect the development and/or course of the disease or condition, and/or in which modulation of KRAS alters the development, course, and/or symptoms. A KRAS mediated disease or condition includes a disease or condition for which KRAS inhibition provides a therapeutic benefit, e.g. wherein treatment with KRAS inhibitors, including compounds described herein, provides a therapeutic benefit to the subject suffering from or at risk of the disease or condition. A KRAS mediated disease or condition is intended to include a cancer that harbors loss of function mutations in KRAS, or a cancer where there is activation of KRAS. A KRAS mediated disease or condition is also intended to include various human carcinomas, including those of colon, lung, pancreas, and ovary, as well as diseases or conditions associated with tumor neovascularization, and invasiveness.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of KRAS. In particular embodiments, the greater specificity is at least 2, 3, 4, 5, 8, 10, 50, 100, 200, 400, 500, or 1000-fold greater specificity.

As used herein in connection with binding compounds or ligands, the term “specific for KRAS,” and terms of like import mean that a particular compound binds to KRAS (e.g., KRAS G12C and/or KRAS G12D) to a statistically greater extent than to other targets that may be present in a particular sample. Also, where biological activity other than binding is indicated, the term “specific for KRAS” indicates that a particular compound has greater biological effect associated with binding KRAS than to other enzymes, e.g., enzyme activity inhibition.

The term “first line cancer therapy” refers to therapy administered to a subject as an initial regimen to reduce the number of cancer cells. First line therapy is also referred to as induction therapy, primary therapy and primary treatment. First-line therapy can be an administered combination with one or more agents. A summary of currently accepted approaches to first line treatment for certain disease can be found in the NCI guidelines for such diseases.

The term “second line cancer therapy” refers to a cancer treatment that is administered to a subject who does not respond to first line therapy, that is, often first line therapy is administered or who has a recurrence of cancer after being in remission. In certain embodiments, second line therapy that may be administered includes a repeat of the initial successful cancer therapy, which may be any of the treatments described under “first line cancer therapy.” A summary of the currently accepted approaches to second line treatment for certain diseases is described in the NCI guidelines for such diseases.

The term “refractory” refers to wherein a subject fails to respond or is otherwise resistant to cancer therapy or treatment. The cancer therapy may be first-line, second-line or any subsequently administered treatment. In certain embodiments, refractory refers to a condition where a subject fails to achieve complete remission after two induction attempts. A subject may be refractory due to a cancer cell's intrinsic resistance to a particular therapy, or the subject may be refractory due to an acquired resistance that develops during the course of a particular therapy.

In addition, abbreviations as used herein have respective meanings as follows:

| | |
|---|---|
| ° C. | Degree Celsius |
| Ac | Acetyl |
| AcOEt or EtOAc | Ethyl Acetate |
| BOC | tert-Butoxycarbonyl |
| DBU | 1,8-Diazabicyclo[5.4.0]undec-7-ene |
| DCM | Dichloromethane |
| DIEA | Diisopropylamine |
| DMF | Dimethylformamide |
| DMSO | Dimethylsulfoxide |
| ESI | Electrospray ionization |
| FBS | Fetal bovine serum |
| HPLC | High Performance Liquid Chromatography |
| IPA | Isopropyl alcohol |
| LAH | Lithium Aluminum Hydride |
| LCMS | Liquid Chromatography Mass Spectrometry |
| [M + H+]+ or (MH)+ | Mass peak plus hydrogen |
| [M − H−]− or (MH)− | Mass peak minus hydrogen |
| Me | Methyl |
| MeOH | Methanol |
| MS | Mass spectrometry |
| PBS | Phosphate buffered saline |
| RT | Room temperature |
| TBS | tert-Butyldimethylsilyl |
| TLC | Thin-layer chromatography |
| THF | Tetrahydrofuran |
| n-Bu | n-Butyl |
| N | Normal |
| IC₅₀ | Half maximal (50%) inhibitory concentration |
| RP | Reverse phase |

### II. Compounds

Compounds contemplated herein are described with reference to both generic formulae and specific compounds. In addition, the compounds described herein may exist in a number of different forms or derivatives, all within the scope of the present disclosure. These include, for example, tautomers, stereoisomers, racemic mixtures, regioisomers, salts, prodrugs (e.g. carboxylic acid esters), solvated forms, and active metabolites.

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present disclosure may exist as stereoisomers as defined herein. All such single stereoisomers, racemates and mixtures thereof are intended to be within the scope of the present disclosure. Unless specified to the contrary, all such stereoisomeric forms are included within the formulae provided herein.

In some embodiments, a chiral compound of the present disclosure is in a form that contains at least 80% of a single isomer (60% enantiomeric excess (“e.e.”) or diastereomeric excess (“d.e.”)), or at least 85% (70% e.e, or d.e.), 90% (80% e.e, or d.e.), 95% (90% e.e, or d.e.), 97.5% (95% e.e, or d.e.), or 99% (98% e.e, or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present disclosure includes both such regioisomers.

In addition to the present formulae and compounds described herein, the disclosure also includes prodrugs (generally pharmaceutically acceptable prodrugs), active metabolic derivatives (active metabolites), and their pharmaceutically acceptable salts.

Unless specified to the contrary, specification of a compound herein includes a pharmaceutically acceptable salt, a solvate, a tautomer, a stereoisomer, or a deuterated analog of such compound.

In some embodiments, compounds of the disclosure are complexed with an acid or a base, including base addition salts such as ammonium, diethylamine, ethanolamine, ethylenediamine, diethanolamine, t-butylamine, piperazine, meglumine; acid addition salts, such as acetate, acetylsalicylate, besylate, camsylate, citrate, formate, fumarate, glutarate, hydrochlorate, maleate, mesylate, nitrate, oxalate, phosphate, succinate, sulfate, tartrate, thiocyanate and tosylate; and amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. In some instances, the amorphous form of the complex is facilitated by additional processing, such as by spray-drying, mechanochemical methods such as roller compaction, or microwave irradiation of the parent compound mixed with the acid or base. Such methods may also include addition of ionic and/or non-ionic polymer systems, including, but not limited to, hydroxypropyl methyl cellulose acetate succinate (HPMCAS) and methacrylic acid copolymer (e.g. Eudragit® L100-55), that further stabilize the amorphous nature of the complex. Such amorphous complexes provide several advantages. For example, lowering of the melting temperature relative to the free base facilitates additional processing, such as hot melt extrusion, to further improve the biopharmaceutical properties of the compound. Also, the amorphous complex is readily friable, which provides improved compression for loading of the solid into capsule or tablet form.

### Compound Embodiments

Embodiment 1(a) of this disclosure relates to a compound having Formula I:

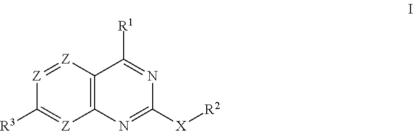

or a pharmaceutically acceptable salt, a solvate, a tautomer, a stereoisomer, or a deuterated analog thereof, wherein:
R¹ is bridged heterocycloalkyl substituted with 0-4 T¹ and 1 G;
R² is alkyl substituted with 0-5 R⁴ and 0-2 J, aryl substituted with 0-4 R⁴ and 0-1 J, arylalkyl substituted with 0-4 R⁴ and 0-1 J, heteroaryl substituted with 0-5 R⁴ and 0-1 J, heteroarylalkyl substituted with 0-5 R⁴ and 0-1 J, heterocycloalkyl substituted with 0-5 R⁴ and 0-1 J, heterocycloalkylalkyl substituted with 0-5 R⁴ and 0-1 J, bridged heterocycloalkyl substituted with 0-5 R⁴ and 0-1 J, bridged heterocycloalkylalkyl substituted with 0-5 R⁴ and 0-1 J, cycloalkyl substituted with 0-5 R⁴ and 0-1 J; or cycloalkylalkyl substituted with 0-5 R⁴ and 0-1 J; provided that when R² is alkyl substituted with 0-5 R⁴ and 0-2 J, X is not —[C(R¹⁰)₂]₁₋₆;
R³ is aryl, heteroaryl, —N(H)-heteroaryl or heterocycloalkyl, wherein R³ is substituted with 0-4 R⁵ and 0-1 M;
each R⁴ is independently alkyl, halogen, hydroxyl, CN, haloalkyl, hydroxyalkyl, C₁-C₆cyanoalkyl, or alkoxy, provided that when R⁴ is attached to a nitrogen atom, R⁴ is not halogen or CN;
each R⁵ is independently alkyl, alkoxy, halogen, hydroxyl, CN, haloalkyl, C₁-C₆cyanoalkyl or hydroxyalkyl, provided that when R⁵ is attached to a nitrogen atom, R⁵ is not halogen or CN;
each R⁶ is independently H, alkyl, haloalkyl or hydroxyalkyl;
each R⁷ is independently H, halogen, alkyl, alkenyl, alkynyl, CN, haloalkyl hydroxyalkyl, cycloalkyl, —C(O)OR¹⁰, or alkoxy optionally substituted with 1-3 halogens;
each R⁸ is independently alkyl, halogen, hydroxyl, —NH₂, —N(H)alkyl, —N(alkyl)₂, CN, SH, haloalkyl or hydroxyalkyl, provided that when R⁸ is attached to nitrogen, R⁸ is not halogen, hydroxyl, —NH₂, —N(H)alkyl, —N(alkyl)₂, or CN;
each R⁹ is independently halogen, hydroxyl, CN, haloalkyl or hydroxyalkyl;
each R¹⁰ is independently H, N(H)₂, alkyl, hydroxyalkyl or haloalkyl;
R¹¹ is cycloalkyl substituted with 0-4 R⁴ and 0-1 J;
each J is independently is —N(R¹⁰)₂, —NO₂, —SO₂N(R¹⁰)₂, —C(O)N(R¹⁰)₂, -alkyl-N(R¹⁰)₂, -alkyl-NHC(O)alkyl, -alkyl-NHC(O)OCH₂-phenyl or —C(O)OR¹⁰; provided that when J is N(R¹⁰)₂, —NO₂, or —SO₂N(R¹⁰)₂, J is not attached to a nitrogen atom;
M is —C(O)NH₂, —N(H)C(O)CH₃, —N(H)SO₂-alkyl, NO₂, or —N(R¹⁰)₂, provided that M is not attached to a nitrogen atom;
X is absent, —[C(R¹⁰)₂]₁₋₆—, —[C[(R¹⁰)₂]₀₋₄—O—, —O—[C(R¹⁰)₂]₁₋₄—, C₂-C₆ alkenylene, or —NR⁶—[C(R¹⁰)₂]₀₋₄—; each Z is independently CR⁷ or N;
G is L¹-L²-T²;
each T¹ is independently alkyl, halogen, hydroxyl, haloalkyl or hydroxyalkyl, provided that when T¹ is attached to nitrogen, T¹ is not halogen or hydroxyl;
T² is H, CN, —C(NH)N(H)CN, —C(NH)OCH₃, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)—SO₂N(R¹⁰)₂, —N(R¹⁰)—C(O)—N(R¹⁰)₂, —N(R¹⁰)C(O)-alkyl, —O—C(O)-alkyl, —C(═NR¹⁰)N(R¹⁰)₂, cycloalkyl, heterocycloalkyl, bridged heterocycloalkyl, heterocyclic spiro ring, carbocyclic spiro ring, aryl or heteroaryl, wherein T² is substituted with 0-4 R⁸ and 0-1 R¹¹ provided that when L¹ and L² are both absent, T² is not CN, —N(R¹⁰)—SO₂N(R¹⁰)₂, —N(R¹⁰)—C(O)—N(R¹⁰)₂, N(R¹⁰)₂, —OR¹⁰, —N(R¹⁰)C(O)-alkyl or —O—C(O)-alkyl, and provided that when L¹ and L² taken together are —C(O), —C(O)C(O)N(H)—, —C(O)O—, —C(O)N(H)—, —S(O)₂—, -alkylene-C(O)N(H)—, -alkylene-C(O)—, —SO₂—, or —SO₂NH—, T² is not CN, —N(R¹⁰)—SO₂N(R¹⁰)₂, —N(R¹⁰)—C(O)—N(R¹⁰)₂, —C(O)OR¹⁰, —N(R¹⁰)₂, —OR¹⁰, —C(O)N(R¹⁰)₂, —SO₂N(R¹⁰)₂, —N(R¹⁰)C(O)-alkyl, —O—C(O)-alkyl, —C(NH)OCH₃, or —C(═NR¹⁰)N(R¹⁰)₂
L¹ is absent, —C(O)—, —C(O)C(O)N(H)—, —C(O)O—, —C(O)N(H)—, —C(O)N(H)—O—, -alkylene-C(O)N(H)—, -alkylene-C(O)—, —SO₂—, or —SO₂N(H)—, provided that when L¹ is —C(O)N(H)—O—, T² is H; and
L² is absent, alkylene substituted with 0-4 R⁹, or C₃-C₆alkenylene substituted with 0-4 R⁹, provided that when L¹ is —C(O)— and L² is absent, T² is not H.

Embodiment 1(b) of this disclosure relates to a compound having Formula I:

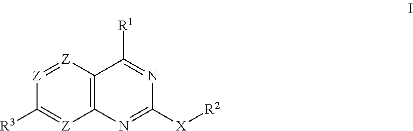

or a pharmaceutically acceptable salt, a solvate, a tautomer, a stereoisomer, or a deuterated analog thereof, wherein:
R¹ is bridged heterocycloalkyl substituted with 0-4 T¹ and 1 G;
R² is alkyl substituted with 0-5 R⁴ and 0-2 J, aryl substituted with 0-4 R⁴ and 0-1 J, arylalkyl substituted with 0-4 R⁴ and 0-1 J, heteroaryl substituted with 0-5 R⁴, heteroarylalkyl substituted with 0-5 R⁴, heterocycloalkyl substituted with 0-5 R⁴, heterocycloalkylalkyl substituted with 0-5 R⁴ and 0-1 J, bridged heterocycloalkyl substituted with 0-5 R⁴, bridged heterocycloalkylalkyl substituted with 0-5 R⁴ and 0-1 J, cycloalkyl substituted with 0-5 R⁴ and 0-1 J, or cycloalkylalkyl substituted with 0-5 R⁴ and 0-1 J; provided that when R² is alkyl substituted with 0-5 R⁴ and 0-2 J, X is not —C[(R¹⁰)₂]₁₋₆;
R³ is aryl, heteroaryl, or heterocycloalkyl, wherein R³ is substituted with 0-4 R⁵ and 0-1 M;
each R⁴ is independently alkyl, halogen, hydroxyl, CN, haloalkyl, hydroxyalkyl, or alkoxy, provided that when R⁴ is attached to a nitrogen atom, R⁴ is not halogen, hydroxyl, or CN;
each R⁵ is independently alkyl, halogen, hydroxyl, CN, haloalkyl, or hydroxyalkyl, provided that when R⁵ is attached to a nitrogen atom, R⁵ is not halogen, hydroxyl, or CN;
each R⁶ is independently H, alkyl, haloalkyl, or hydroxyalkyl;
each R⁷ is independently H, halogen, alkyl, alkenyl, alkynyl, CN, haloalkyl, hydroxyalkyl, cycloalkyl, —C(O)OR¹⁰, or alkoxy optionally substituted with 1-3 halogens;
each R⁸ is independently alkyl, halogen, hydroxyl, —NH₂, CN, haloalkyl, or hydroxyalkyl, provided that when R⁸ is attached to nitrogen, R⁸ is not halogen, hydroxyl, —NH₂ or CN;
each R⁹ is independently halogen, hydroxyl, CN, haloalkyl, or hydroxyalkyl;
each R¹⁰ is independently H, alkyl, hydroxyalkyl, or haloalkyl;
each J is independently is —N(R¹⁰)₂, —C(O)N(R¹⁰)₂, or —C(O)OR¹⁰;
M is —C(O)NH₂, —NH₂C(O)CH₃, —N(H)SO₂-alkyl, or —N(R¹⁰)₂;
X is absent, —[C(R¹⁰)₂]₁₋₆—, —[C(R¹⁰)₂]₀₋₃—O—, or —NR⁶—;
each Z is independently CR⁷ or N;
G is L¹-L²-T²;
each T¹ is independently alkyl halogen hydroxyl, haloalkyl, or hydroxyalkyl, provided that when T¹ is attached to nitrogen, T¹ is not halogen or hydroxyl;
T² is H, —C(O)OR¹⁰, N(R¹⁰)₂, —OR¹⁰, —C(O)N(R¹⁰)₂, —SO₂N(R¹⁰)₂, —N(R¹⁰)C(O)-alkyl, —O—C(O)-alkyl, C(═NR¹⁰)N(R¹⁰)₂, cycloalkyl, heterocycloalkyl, bridged heterocycloalkyl, heterocyclic spiro ring, carbocyclic spiro ring, aryl, or heteroaryl, wherein T² is substituted with 0-4 R⁸, provided that when L¹ and L² are both absent, T² is not N(R¹⁰)₂, —OR¹⁰, —N(R¹⁰)C(O)-alkyl, or —O—C(O)-alkyl;
L¹ is absent, —C(O)—, —C(O)C(O)N(H)—, —C(O)O—, —C(O)N(H)—, —S(O)₂—, -alkylene-C(O)N(H)—, -alkylene-C(O)—, —SO₂—, or —SO₂NH—; and
L² is absent, or alkylene substituted with 0-4 R⁹, provided that when L¹ is —C(O)— and L² is absent, T² is not H.
Embodiment 1(c) relates to Embodiment 1(a) wherein G is H.
Embodiment 1(d) relates to Embodiment 1(a) wherein G is not H.
Embodiment 1(e) relates to Embodiment 1(b) wherein G is H.
Embodiment 1(f) relates to Embodiment 1(b) wherein G is not H.

Embodiment 2(a) of this disclosure relates to the compound of Embodiment 1(a), wherein:
X is absent, —[C(R¹⁰)₂]₁₋₄—, —(CH₂)₀₋₃—O—, O—[C(R¹⁰)₂]₁₋₃—, C₃-C₄ alkenylene, or —NR⁶—;
R¹ is a 6-9 membered bridged heterocycloalkyl having at least one nitrogen atom, wherein R¹ is substituted with 0-4 T¹ and 1 G;
R² is C₁-C₆alkyl substituted with 0-5 R⁴ and 0-2 J, phenyl substituted with 0-4 R⁴ and 0-1 J, —C₀-C₆alkyl-phenyl substituted with 0-4 R⁴ and 0-1 J, 5-10 membered heteroaryl substituted with 0-5 R⁴ and 0-1 J, —C₀-C₆alkyl-5-10 membered heteroaryl substituted with 0-5 R⁴ and 0-1 J, —C₀-C₆alkyl-4-10 membered heterocycloalkyl substituted with 0-5 R⁴ and 0-1 J, or —C₀-C₆alkyl-C₃-C₁₀cycloalkyl substituted with 0-5 R⁴ and 0-1 J, provided that when R² is C₁-C₆alkyl substituted with 0-5 R⁴ and 0-2 J, X is not-C[(R¹⁰)₂]₁₋₃;
R³ is phenyl, naphthyl, 5-10 membered heteroaryl, or 5-10 membered heterocycloalkyl, wherein R³ is substituted with 0-4 R⁵ and 0-1 M;
each R⁴ is independently C₁-C₆alkyl, halogen, hydroxyl, CN, C₁-C₆haloalkyl, C₁-C₆hydroxyalkyl, C₁-C₆cyanoalkyl, or C₁-C₆alkoxy, provided that when R⁴ is attached to a nitrogen atom, R⁴ is not halogen, or CN;
each R⁵ is independently C₁-C₆alkyl. C₁-C₆alkoxy, halogen, hydroxyl, CN, C₁-C₆haloalkyl, C₁-C₆cynoalkyl, or C₁-C₆hydroxyalkyl, provided that when R⁵ is attached to a nitrogen atom, R⁵ is not halogen or CN;
each R⁶ is independently H, C₁-C₆alkyl. C₁-C₆haloalkyl or C₁-C₆hydroxyalkyl;
each R⁷ is independently H, halogen, C₁-C₄alkyl, C₂-C₄alkenyl, C₂-C₄alkynyl, CN, C₁-C₄haloalkyl, C₁-C₄hydroxyalkyl; C₃-C₄cycloalkyl, —C(O)OR¹⁰, or C₁-C₄alkoxy optionally substituted with 1-3 halogens;
each R⁸ is independently C₁-C₆alkyl, halogen, hydroxyl, —NH₂, —N(H)C₁-C₆alkyl, —N(C₁-C₆alkyl)₂, CN, C₁-C₆haloalkyl, or C₁-C₆hydroxyalkyl, provided that when R⁸ is attached to nitrogen, R⁸ is not halogen, hydroxyl, —NH₂, —N(H)C₁-C₆alkyl. —N(C₁-C₆alkyl)₂, or CN;
each R⁹ is independently halogen, hydroxyl, CN, C₁-C₆haloalkyl, or C₁-C₆hydroxyalkyl;
each R¹⁰ is independently H, —N(H)₂, C₁-C₆alkyl, or C₁-C₆1haloalkyl:
R¹¹ is C₃-C₆cycloalkyl substituted with 0-3 R⁴ and 0-1 J; each J is independently is —N(R¹⁰)₂, —SO₂N(R¹⁰)₂, —C(O)N(R¹⁰)₂, or —C(O)OR¹⁰; -provided that when J is —N(R¹⁰)₂, or —SO₂N(R¹⁰)₂, J is not attached to a nitrogen atom;
each T¹ is independently C₁-C₆alkyl, halogen, hydroxyl, C₁-C₆haloalkyl, or C₁-C₆hydroxyalkyl, provided that when T¹ is attached to nitrogen, T¹ is not halogen or hydroxyl;
T² is H, CN, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)C(O)—C₁-C₆alkyl, —O—C(O)— C₁-C₆alkyl. —C(═NH)NH₂, C₃-C₆cycloalkyl, 4-10 membered heterocycloalkyl, 5-9 membered bridged heterocycloalkyl, 6-9 membered heterocyclic spiro ring, 6-9 membered carbocyclic spiro ring, phenyl, naphthyl or 5-10 membered heteroaryl, wherein T² is substituted with 0-4 R⁸ and 0-1 R¹¹; provided that when L¹ and L² are both absent, T² is not CN, N(R¹⁰)₂, —OR¹⁰, —N(R¹⁰)C(O)—C₁-C₆alkyl or —O—C(O)—C₁-C₆alkyl, and provided that when L¹ and L² taken together are —C(O)—, —C(O)C(O)N(H)—, —C(O)O—, —C(O)N(H)—, —C(O)N(H)—O—, —C₁-C₆alkyelene-C(O)N(H)—, —C₁-C₆alkylene-C(O)—, —SO₂—, or —SO₂NH—, T² is not CN, —C(O)OR¹⁰, C₁-C₆alkoxy, N(R¹⁰)₂, —OR¹⁰, —C(O)N(R¹⁰)₂, —SO₂N(R¹⁰)₂, —N(R¹⁰)C(O)—C₁-C₆alkyl, —O—C(O)—C₁-C₆alkyl, or —C(═NH)NH₂;
L¹ is absent, —C(O)—, —C(O)C(O)N(H)—, —C(O)O—, —C(O)N(H)—, —C₁-C₆alkyelene-C(O)N(H)—, —C₁-C₆alkylene-C(O)—, —SO₂—, or —SO₂N(H)— provided that when L¹ is —C(O)N(H)—O—, T² is H;
L² is absent, C₁-C₆alkylene substituted with 0-2 R⁹, or C₃-C₆alkenylene substituted with 0-4 R⁹, provided that when L¹ is —C(O)— and L² is absent, T² is not H; and
M is —C(O)NH₂, —NH₂C(O)CH₃, —N(H)SO₂—C₁-C₃alkyl, or —NH₂, provided that M is not attached to a nitrogen atom.

Embodiment 2(b) of this disclosure relates to the compound of Embodiment 1(b), wherein:
X is absent, —C[(R¹⁰)₂]₁₋₃—, —(CH₂)₀₋₂—O—, or —NR⁶—
R¹ is a 6-9 membered bridged heterocycloalkyl having at least one nitrogen atom, wherein R¹ is substituted with 0-4 T¹ and 1 G;
R² is C₁-C₆alkyl substituted with 0-5 R⁴ and 0-2 J, phenyl substituted with 0-4 R⁴ and 0-1 J, —C₀-C₆alkyl-phenyl substituted with 0-4 R⁴ and 0-1 J, 5-10 membered heteroaryl substituted with 0-5 R⁴, —C₀-C₆alkyl-5-10 membered heteroaryl substituted with 0-5 R⁴, —C₀-C₆alkyl-4-10 membered heterocycloalkyl substituted with 0-5 R⁴, or —C₀-C₆alkyl-C₃-C₁₀cycloalkyl substituted with 0-5 R⁴ and 0-1 J, provided that when R² is C₁-C₆alkyl substituted with 0-5 R⁴ and 0-2 J, X is not-C[(R¹⁰)₂]₁₋₃;
R³ is phenyl, naphthyl, 5-10 membered heteroaryl, or 5-10 membered heterocycloalkyl, wherein R³ is substituted with 0-4 R⁵ and 0-1 M;
each R⁴ is independently C₁-C₆alkyl, halogen, hydroxyl, CN, C₁-C₆haloalkyl. C₁-C₆hydroxyalkyl, or C₁-C₆alkoxy, provided that when R⁴ is attached to a nitrogen atom, R⁴ is not halogen, hydroxyl, or CN;
each R⁵ is independently C₁-C₆alkyl, halogen, hydroxyl, CN, C₁-C₆haloalkyl, or C₁-C₆hydroxyalkyl, provided that when R⁵ is attached to a nitrogen atom, R⁴ is not halogen, hydroxyl, or CN;
each R⁶ is independently H, C₁-C₆alkyl. C₁-C₆haloalkyl, or C₁-C₆hydroxyalkyl;
each R⁷ is independently H, halogen, C₁-C₄alkyl. C₂-C₄alkenyl, C₂-C₄alkynyl, CN, C₁-C₄haloalkyl. C₁-C₄hydroxyalkyl; C₃-C₄cycloalkyl, —C(O)OR¹⁰, or C₁-C₄alkoxy optionally substituted with 1-3 halogens;
each R⁸ is independently C₁-C₆alkyl, halogen, hydroxyl, —NH₂, CN, C₁-C₆haloalkyl, or C₁-C₆hydroxyalkyl, provided that when R⁸ is attached to nitrogen, R⁸ is not halogen, hydroxyl, —NH₂, or CN;
each R⁹ is independently halogen, hydroxyl, CN, C₁-C₆haloalkyl, or C₁-C₆hydroxyalkyl;
each R¹⁰ is independently H, C₁-C₆alkyl, or C₁-C₆1haloalkyl;
each T¹ is independently C₁-C₆alkyl halogen hydroxyl, C₁-C₆haloalkyl, or C₁-C₆hydroxyalkyl, provided that when T¹ is attached to nitrogen, T¹ is not halogen or hydroxyl;
T² is H, —C(O)OR¹⁰, C₁-C₆alkoxy, N(R¹⁰)₂, —OR¹⁰, —C(O)N(R¹⁰)₂, —SO₂N(R¹⁰)₂, —N(R¹⁰)C(O)—C₁-C₆alkyl, —O—C(O)—C₁-C₆alkyl, —C(═NH)NH₂, C₃-C₆cycloalkyl, 4-10 membered heterocycloalkyl, 5-9 membered bridged heterocycloalkyl, 6-9 membered heterocyclic spiro ring, 6-9 membered carbocyclic spiro ring, phenyl, naphthyl or 5-10 membered heteroaryl, wherein T² is substituted with 0-4 R⁸, provided that when
L¹ and L² are both absent, T² is not N(R¹⁰)₂, —OR¹⁰, —N(R¹⁰)C(O)—C₁-C₆alkyl, or —O—C(O)—C₁-C₆alky 1;
L¹ is absent, —C(O)—, —C(O)C(O)N(H)—, —C(O)O—, —C(O)N(H)—, —C₁-C₆alkyelene-C(O)N(H)—, —C₁-C₆alkylene-C(O)—, —SO₂—, or —SO₂NH—;
L² is absent, or C₁-C₆alkylene substituted with 0-4 R⁹, provided that when L¹ is —C(O)— and L² is absent, T² is not H; and
M is —C(O)NH₂, —NH₂C(O)CH₃, —N(H)SO₂— C₁-C₃alkyl, or —NH₂.
Embodiment 2(c) relates to the compound of Embodiment 2(a) wherein G is H.
Embodiment 2(d) relates to the compound of Embodiment 2(a) wherein G is not H.
Embodiment 2(e) relates to the compound of Embodiment 2(b) wherein G is H.
Embodiment 2(f) relates to the compound of Embodiment 2(b) wherein G is not H.

Embodiment 3 of this disclosure relates to the compound of any of Embodiments 1(a), 1(b), 2(a) or 2(b) having one of the following formulae:

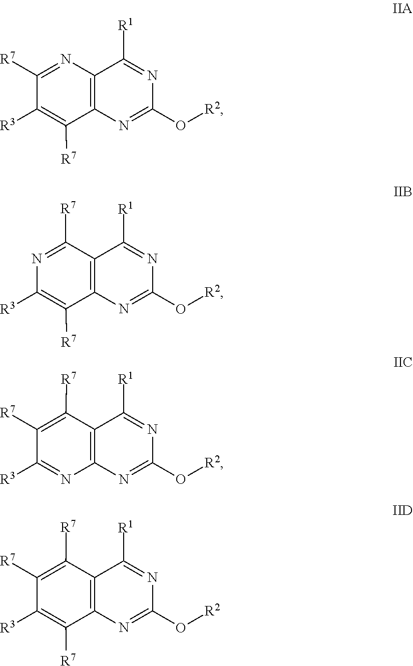

or a pharmaceutically acceptable salt, a solvate, a tautomer, a stereoisomer, or a deuterated analog thereof.
Embodiment 3(a) relates to the compound of Embodiment 3 wherein G is H.
Embodiment 3(b) relates to the compound of Embodiment 3 wherein G is not H.

Embodiment 4 of this disclosure relates to the compound of Embodiment 3 having Formula IIB or IID, or a pharmaceutically acceptable salt, a solvate, a tautomer, a stereoisomer, or a deuterated analog thereof.

Embodiment 4(a) relates to the compound of Embodiment 4 wherein G is H.

Embodiment 4(b) relates to the compound of Embodiment 4 wherein G is not H.

Embodiment 5 of this disclosure relates to the compound according to the compound of any one of Embodiments 1(a), 1(b), 2(a), 2(b), 3, or 4 having one of the following formulae:

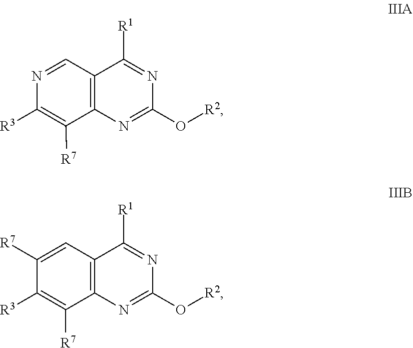

or a pharmaceutically acceptable salt, a solvate, a tautomer, a stereoisomer, or a deuterated analog thereof, wherein each R⁷ is independently H, F, Cl, C₁-C₃alkyl, or C₁-C₃haloalkyl.
Embodiment 5(a) relates to the compound of Embodiment 5 wherein G is H.
Embodiment 5(b) relates to the compound of Embodiment 5 wherein G is not H.

Embodiment 6 of this disclosure relates to the compound according to any one of Embodiments 1(a), 1(b), 2(a), 2(b), 3, 4 or 5 having one of the following formulae:

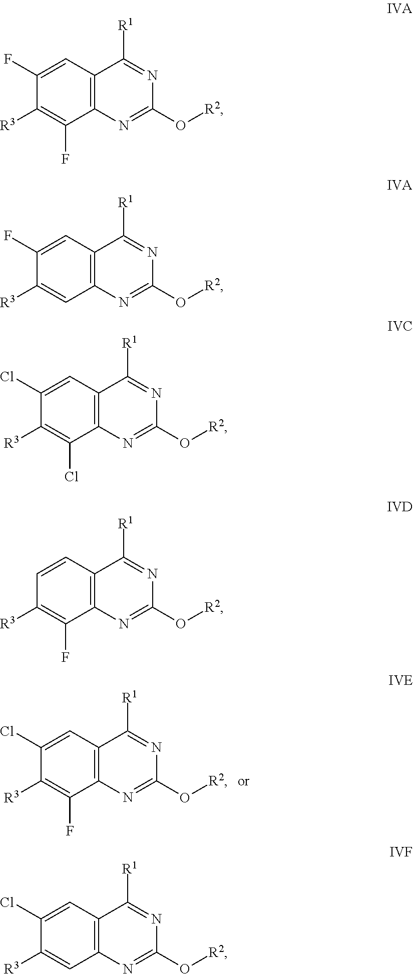

or a pharmaceutically acceptable salt, a solvate, a tautomer, a stereoisomer, or a deuterated analog thereof.
Embodiment 6(a) relates to the compound of Embodiment 6 wherein G is H.
Embodiment 6(b) relates to the compound of Embodiment 6 wherein G is not H.

Embodiment 7(a) of this disclosure relates to the compound according to any one of Embodiments 1(a), 2(a), 3, 4, 5 or 6 wherein:
R¹ is a 7-9 membered bridged heterocycloalkyl having at least one nitrogen atom, wherein R¹ is substituted with 0-3 T¹ and 1 G;
R² is —C₀-C₅alkyl-4-10 membered heterocycloalkyl substituted with 0-4 R⁴ and 0-1 J; —C₀-C₅alkyl-5-10 membered heteroaryl substituted with 0-3 R⁴ and 0-1 J; or C₁-C₆alkyl substituted with 1-2 J groups;
R³ is phenyl, naphthyl, 5-10 membered heteroaryl, or 5-10 membered heterocycloalkyl, wherein the naphthyl, 5-10 membered heteroaryl, and 5-10 membered heterocycloalkyl are each substituted with 0-4 R⁵, and wherein the phenyl is substituted with 0-4 R⁵ and 0-1M;
each R⁴ is independently C₁-C₅alkyl, halogen, hydroxyl, CN, C₁-C₅haloalkyl, C₁-C₅hydroxyalkyl, C₁-C₅cyanoalkyl or C₁-C₅alkoxy, provided that when R⁴ is attached to a nitrogen atom, R⁴ is not halogen, hydroxyl, or CN;
each R⁵ is independently C₁-C₅alkyl, C₁-C₅alkoxy, halogen, hydroxyl, CN, C₁-C₅haloalkyl. C₁-C₅cynoalkyl, or C₁-C₅hydroxyalkyl;
each R⁸ is independently C₁-C₅alkyl, halogen, hydroxyl, —NH₂, —N(H)C₁-C₅alkyl, —N(C₁-C₅alkyl)₂-CN, C₁-C₅haloalkyl or C₁-C₅hydroxyalkyl, provided that when R⁸ is attached to nitrogen, R⁸ is not halogen, hydroxyl, —NH₂, —N(H)C₁-C₅alkyl-N(C₁-C₅alkyl)₂, or CN;
each R⁹ is independently halogen, hydroxyl, CN, C₁-C₅haloalkyl, or C₁-C₅hydroxyalkyl;
each R¹⁰ is independently H, C₁-C₅alkyl, or C₁-C₅haloalkyl;
each T¹ is independently C₁-C₅alkyl, halogen, hydroxyl, C₁-C₅haloalkyl or C₁-C₅hydroxyalkyl, provided that when T¹ is attached to nitrogen, T¹ is not halogen or hydroxyl;
T² is H, CN, —C(O)OR¹⁰, C₁-C₅alkoxy, N(R¹⁰)₂, —C(O)N(R¹⁰)₂, —SO₂N(R¹⁰)₂, —SO₂R¹⁰,
—N(R¹⁰)C(O)—C₁-C₅alkyl, —O—C(O)—C₁-C₅alkyl, C₃-C₆cycloalkyl, 4-10 membered heterocycloalkyl, 7-9 membered bridged heterocycloalkyl, phenyl, naphthyl or 5-10 membered heteroaryl, wherein T² substituted with 0-4 R⁸, provided that when L¹ and L² are both absent, T² is not CN, C₁-C₅alkoxy. N(R¹⁰)₂,
—N(R¹⁰)C(O)—C₁-C₅alkyl or —O—C(O)—C₁-C₅alkyl, and provided that when L¹ and L² taken together are —C(O)—, —C(O)N(H)—, —C₁-C₅alkyelene-C(O)N(H)—, —C₁-C₅alkylene-C(O)—, —SO₂—, or
—SO₂NH, T² is not CN, —C(O)OR¹⁰, C₁-C₅alkoxy, N(R¹⁰)₂, —C(O)N(R¹⁰)₂, —SO₂N(R¹⁰)₂, —N(R¹⁰)C(O)—C₁-C₅alkyl, or —O—C(O)—C₁-C₅alkyl;
L¹ is absent, —C(O)—, —C(O)N(H)—, —C₁-C₅alkyelene-C(O)N(H)—, —C₁-C₅alkylene-C(O)—, —SO₂—, or —SO₂N(H)—; and
L² is absent, or C₁-C₅alkylene substituted with 0-4 R⁹, provided that when L¹ is —C(O)— and L² is absent, T² is not H; and
M is —C(O)NH₂, —NH₂C(O)CH₃, —N(H)SO₂CH₃, or —NH₂, provided that M is not attached to a nitrogen atom.

Embodiment 7(b) of this disclosure relates to the compound according to any one of Embodiments 1(b), 2(b), 3, 4, 5 or 6, wherein:
R¹ is a 7-9 membered bridged heterocycloalkyl having at least one nitrogen atom, wherein R¹ is substituted with 0-3 T¹ and 1 G;
R² is —C₀-C₅alkyl-4-10 membered heterocycloalkyl substituted with 0-4 R⁴; —C₀-C₅alkyl-5-10 membered heteroaryl substituted with 0-3 R⁴; or C₁-C₆alkyl substituted with 1-2 J groups;
R³ is phenyl, naphthyl, 5-10 membered heteroaryl, or 5-10 membered heterocycloalkyl, wherein the naphthyl, 5-10 membered heteroaryl, and 5-10 membered heterocycloalkyl are each substituted with 0-4
R⁵, and wherein the phenyl is substituted with 0-4 R⁵ and 0-1M; each R⁴ is independently C₁-C₅alkyl, halogen, hydroxyl, CN, C₁-C₅haloalkyl. C₁-C₅hydroxyalkyl or C₁-C₅alkoxy, provided that when R⁴ is attached to a nitrogen atom, R⁴ is not halogen, hydroxyl, or CN;
each R⁵ is independently C₁-C₅alkyl, halogen, hydroxyl, CN, C₁-C₅haloalkyl, or C₁-C₅hydroxyalkyl;
each R⁸ is independently C₁-C₅alkyl, halogen, hydroxyl, CN, C₁-C₅haloalkyl, or C₁-C₅hydroxy alkyl, provided that when R⁸ is attached to nitrogen, R⁸ is not halogen, hydroxyl, or CN;
each R⁹ is independently halogen, hydroxyl, CN, C₁-C₅haloalkyl, or C₁-C₅hydroxy alkyl;
each R¹⁰ is independently H, C₁-C₅alkyl, or C₁-C₅haloalkyl;
each T¹ is independently C₁-C₅alkyl halogen hydroxyl, C₁-C₅haloalkyl or C₁-C₅hydroxy alkyl, provided that when T¹ is attached to nitrogen, T¹ is not halogen or hydroxyl;
T² is H, —C(O)OR¹⁰, C₁-C₅alkoxy, N(R¹⁰)₂, —C(O)N(R¹⁰)₂, —SO₂N(R¹⁰)₂, —N(R¹⁰)C(O)—C₁-C₅alkyl, —O—C(O)—C₁-C₅alkyl, C₃-C₆cycloalkyl, 4-10 membered heterocycloalkyl, 7-9 membered bridged heterocycloalkyl, phenyl, naphthyl or 5-10 membered heteroaryl, wherein T² is substituted with 0-4 R⁸, provided that when L¹ and L² are both absent, T² is not C₁-C₅alkoxy, N(R¹⁰)₂, —N(R¹⁰)C(O)—C₁-C₅alkyl or —O—C(O)—C₁-C₅alkyl;
L¹ is absent, —C(O)—, —C(O)N(H)—, —C₁-C₅alkyeleneC(O)N(H)—, —C₁-C₅alkylene-C(O)—, —SO₂—, or —SO₂NH—; and
L² is absent, or C₁-C₅alkylene substituted with 0-4 R⁹, provided that when L¹ is —C(O)— and L² is absent, T² is not H; and
M is —C(O)NH₂, —NH₂C(O)CH₃, —N(H)SO₂CH₃, or —NH₂.
Embodiment 7(c) relates to the compound of Embodiment 7(a) wherein G is H.
Embodiment 7(d) relates to the compound of Embodiment 7(a) wherein G is not H.
Embodiment 7(e) relates to the compound of Embodiment 7(b) wherein G is H.
Embodiment 7(f) relates to the compound of Embodiment 7(b) wherein G is not H.

Embodiment 8(a) of this disclosure relates to the compound according to any of Embodiments 1(a), 2(a), 3, 4, 5, 6 or 7(a), wherein:
R¹ is a 7-9 membered bridged heterocycloalkyl having at least one nitrogen atom, wherein R¹ is substituted with 0-2 T¹ and 1 G;
R² is 5-10 membered heterocyloalkyl-C₁-C₄alkyl substituted with 0-3 R⁴, or C₁-C₅alkyl substituted with 1-2 J groups;
R³ is phenyl, naphthyl 5-10 membered heteroaryl, or a 5-10 membered heterocycloalkyl, wherein the naphthyl, 5-10 membered heteroaryl, and 5-10 membered heterocycloalkyl are each substituted with 0-3 R⁵, and wherein the phenyl is substituted with 0-4 R⁵ and 0-1M;
each R⁴ is independently C₁-C₄alkyl, halogen, hydroxyl, CN, C₁-C₄haloalkyl. C₁-C₄hydroxyalkyl or C₁-C₄alkoxy, provided that when R⁴ is attached to a nitrogen atom, R⁴ is not halogen, hydroxyl, or CN;
each R⁵ is independently C₁-C₄alkyl, halogen, hydroxyl, CN, C₁-C₄haloalkyl. C₁-C₄cynoalkyl or C₁-C₄hydroxyalkyl;
each R⁸ is independently C₁-C₄alkyl, halogen, hydroxyl, —NH₂, —N(H)C₁-C₄alkyl. —N(C₁-C₄alkyl)₂, CN, C₁-C₄haloalkyl or C₁-C₄hydroxyalkyl, provided that when R⁸ is attached to nitrogen, R⁸ is not halogen, hydroxyl, —NH₂, —N(H)C₁-C₅alkyl-N(C₁-C₅alkyl)₂, or CN;
each R⁹ is independently halogen, hydroxyl, CN, C₁-C₄haloalkyl or C₁-C₄hydroxyalkyl;
each R¹⁰ is independently H, C₁-C₄alkyl, or C₁-C₄haloalkyl; each T¹ is independently C₁-Chalky 1 halogen hydroxyl, C₁-C₄haloalkyl, or C₁-C₄hydroxyalkyl, provided that when T¹ is attached to nitrogen, T¹ is not halogen or hydroxyl;
T² is H, CN, —C(O)OR¹⁰, C₁-C₄alkoxy. —N(R¹⁰)₂, —C(O)N(R¹⁰)₂, SO₂N(R¹⁰)₂, —SO₂R¹⁰, —N(R¹⁰)C(O)—C₁-C₄alkyl, —O—C(O)—C₁-C₄alkyl, C₃-C₆cycloalkyl, 4-6 membered heterocycloalkyl, 7-8 membered bridged heterocycloalkyl, phenyl, naphthyl or 5-6 membered heteroaryl, wherein T² is substituted with 0-4 R⁸,
provided that when L¹ and L² are both absent, T² is not CN, C₁-C₄alkoxy. N(R¹⁰)₂,
—N(R¹⁰)C(O)—C₁-C₄alkyl, or —O—C(O)—C₁-C₄alkyl, and provided that when L¹ and L² taken together are —C(O)—, —C(O)N(H)—, —S(O)₂—, C₁-C₄alkyelene-C(O)N(H)—, or —C₁-C₄alkylene-C(O)—. T² is not CN, —C(O)OR¹⁰, C₁-C₄alkoxy. —N(R¹⁰)₂, —C(O)N(R¹⁰)₂, SO₂N(R¹⁰)₂, —N(R¹⁰)C(O)—C₁-C₄alkyl, or —O—C(O)—C₁-C₄alkyl;
L¹ is absent, —C(O)—, —C(O)N(H)—, —S(O)₂—, —SO₂N(H)—, C₁-C₄alkyelene-C(O)N(H)—, C₁-C₄alkylene-C(O)—, or —C₁-C₄alkylene-C(O)—; and
L² is absent, or C₁-C₄alkylene substituted with 0-2 R⁹, provided that when L¹ is —C(O)— and L² is absent, T² is not H.

Embodiment 8(b) of this disclosure relates to the compound according to any of Embodiments 1(b), 2(b), 3, 4, 5, 6 or 7(b), wherein:
R¹ is a 7-9 membered bridged heterocycloalkyl having at least one nitrogen atom, wherein R¹ is substituted with 0-2 T¹ and 1 G;
R² is 5-10 membered heterocyloalkyl-C₁-C₄alkyl substituted with 0-3 R⁴ or C₁-C₄alky 1 substituted with 1-2 J groups;
R³ is phenyl, naphthyl 5-10 membered heteroaryl, or a 5-10 membered heterocycloalkyl, wherein the naphthyl, 5-10 membered heteroaryl, and 5-10 membered heterocycloalkyl are each substituted with 0-3 R⁵, and wherein the phenyl is substituted with 0-4 R⁵ and 0-1M;
each R⁴ is independently C₁-C₄alkyl, halogen, hydroxyl, CN, C₁-C₄haloalkyl, C₁-C₄hydroxyalkyl, C₁-C₄cyanoalkyl or C₁-C₄alkoxy, provided that when R⁴ is attached to a nitrogen atom, R⁴ is not halogen, hydroxyl or CN;
each R⁵ is independently C₁-C₄alkyl, halogen, hydroxyl, CN, C₁-C₄haloalkyl, or C₁-C₄hydroxyalkyl;
each R⁸ is independently C₁-C₄alkyl, halogen, hydroxyl, CN, C₁-C₄haloalkyl, or C₁-C₄hydroxyalkyl, provided that when R⁸ is attached to nitrogen, R⁸ is not halogen, hydroxyl, or CN;
each R⁹ is independently halogen, hydroxyl, CN, C₁-C₄haloalkyl, or C₁-C₄hydroxyalkyl;
each R¹⁰ is independently H, C₁-C₄alkyl, or C₁-C₄haloalkyl;
each T¹ is independently C₁-C₄alkyl halogen hydroxyl, C₁-C₄haloalkyl, or C₁-C₄hydroxyalkyl, provided that when T¹ is attached to nitrogen, T¹ is not halogen or hydroxyl;
T² is H, —C(O)OR¹⁰, C₁-C₄alkoxy, —N(R¹⁰)₂, —C(O)N(R¹⁰)₂, SO₂N(R¹⁰)₂, —N(R¹⁰)C(O)— C₁-C₆alkyl, —O—C(O)—C₁-C₆alkyl, C₃-C₆cycloalkyl, 4-6 membered heterocycloalkyl, 7-8 membered bridged heterocycloalkyl, phenyl, naphthyl or 5-6 membered heteroaryl, wherein T² is substituted with 0-4 R⁸, provided that when L¹ and L² are both absent, T² is not C₁-C₄alkoxy, N(R¹⁰)₂, —N(R¹⁰)C(O)—C₁-C₄alkyl, or —O—C(O)—C₁-C₄alkyl;
L¹ is absent, —C(O)—, —C(O)N(H)—, —S(O)₂—, C₁-C₄alkyelene-C(O)N(H)—, or —C₁-C₄alkylene-C(O)—; and
L² is absent, or C₁-C₄alkylene substituted with 0-2 R⁹, provided that when L¹ is —C(O)— and L² is absent, T² is not H.
Embodiment 8(c) relates to the compound of Embodiment 8(a) wherein G is H.
Embodiment 8(d) relates to the compound of Embodiment 8(a) wherein G is not H.
Embodiment 8(e) relates to the compound of Embodiment 8(b) wherein G is H.
Embodiment 8(f) relates to the compound of Embodiment 8(b) wherein G is not H.

Embodiment 9 of this disclosure relates to the compound according to any one of Embodiments 1(a), 1(b), 2(a), 2(b), 3, 4, 5, 6, 7(a), 7(b), 8(a) or 8(b), wherein R¹ is 7-9 membered bridged piperazine or piperidine substituted with 0-2 T¹ and 1 G.

Embodiment 9(a) relates to the compound of Embodiment 9 wherein G is H.

Embodiment 9(b) relates to the compound of Embodiment 9 wherein G is not H.

Embodiment 10(a) of this disclosure relates to the compound according to any one of Embodiments 1(a), 2(a), 3, 4, 5, 6, 7(a), or 8(a), wherein RGs one of the following formulae:

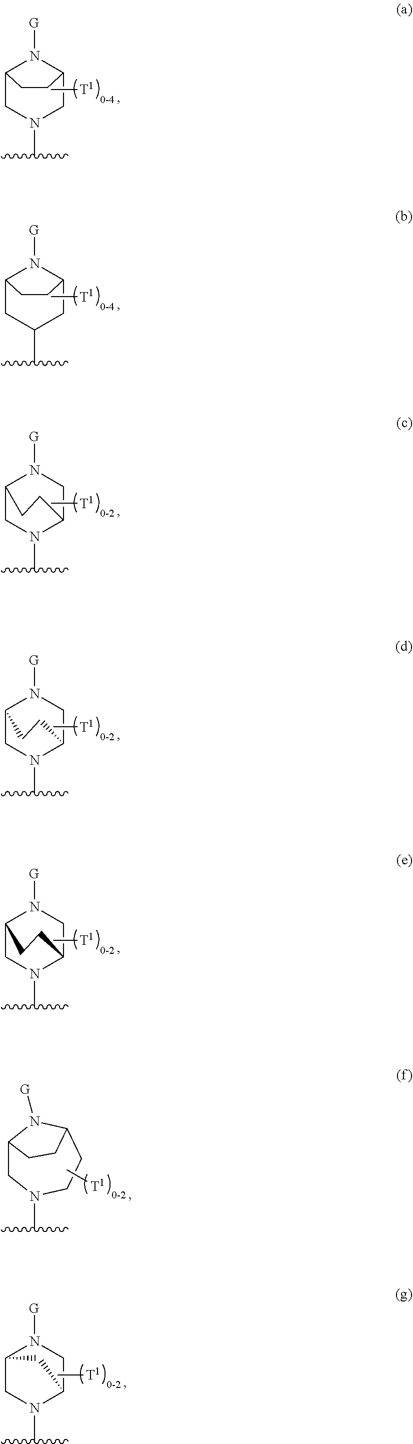

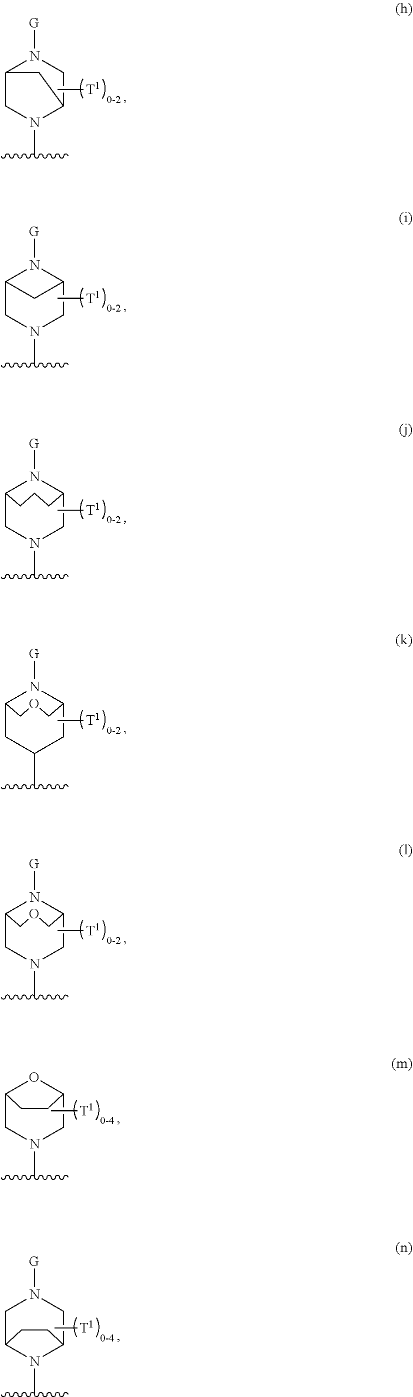

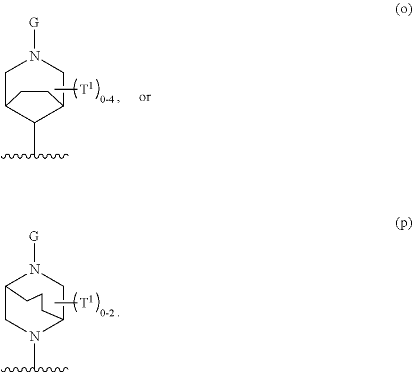

Embodiment 10(b) of this disclosure relates to the compound according to any one of Embodiments 1(b), 2(b), 3, 4, 5, 6, 7(b), or 8(b), wherein R¹ is one of the following formulae:

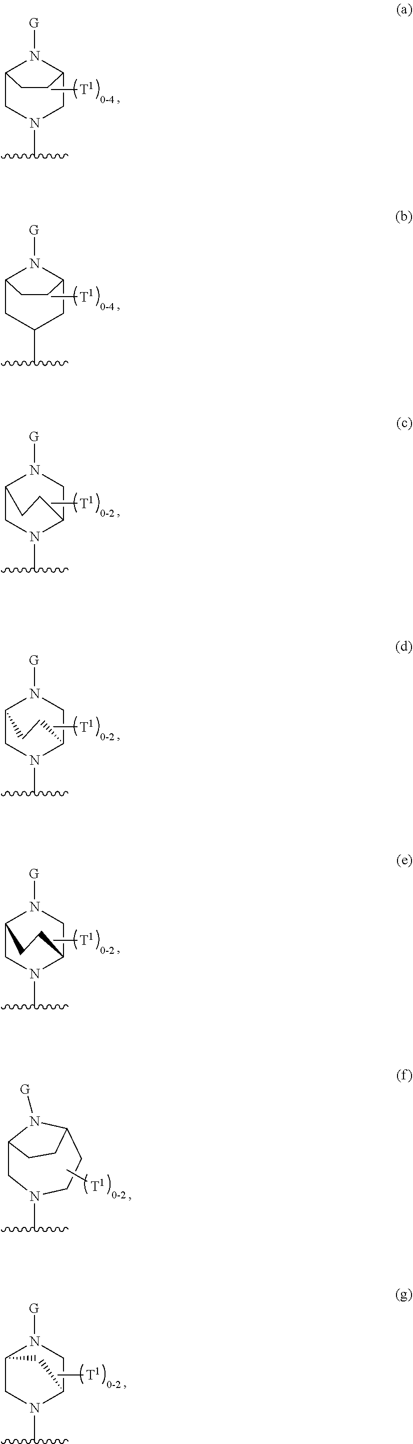

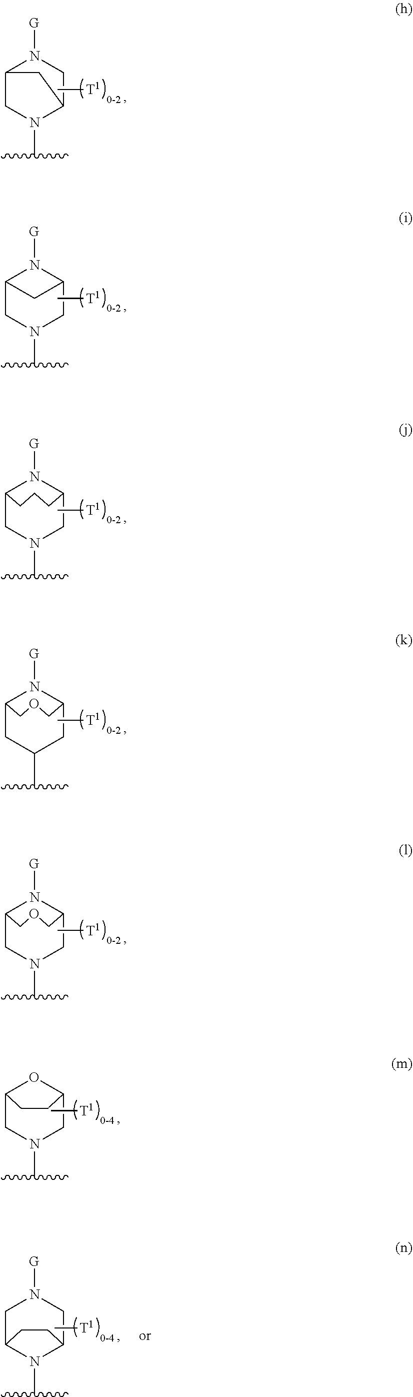

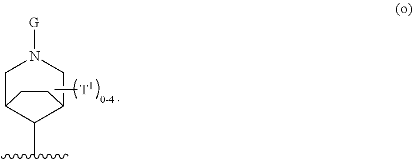

Embodiment 10(c) relates to the compound of Embodiment 10(a) wherein G is H.
Embodiment 10(d) relates to the compound of Embodiment 10(a) wherein G is not H.
Embodiment 10(e) relates to the compound of Embodiment 10(b) wherein G is H.
Embodiment 10(f) relates to the compound of Embodiment 10(b) wherein G is not H.

Embodiment 11(a) of this disclosure relates to the compound according to Embodiment 10(a), wherein R¹ is formula (a) or (b).

Embodiment 11(b) of this disclosure relates to the compound according to Embodiment 10(b), wherein R¹ is formula (a) or (b).

Embodiment 11(c) relates to the compound of Embodiment 11(a) wherein G is H.

Embodiment 11(d) relates to the compound of Embodiment 11(a) wherein G is not H.

Embodiment 11(e) relates to the compound of Embodiment 11(b) wherein G is H.

Embodiment 11(f) relates to the compound of Embodiment 11(b) wherein G is not H.

Embodiment 12(a) of this disclosure relates to the compound according to any one of Embodiments 1(a), 2(a), 3, 4, or 5, wherein R² is one of the following formulae:

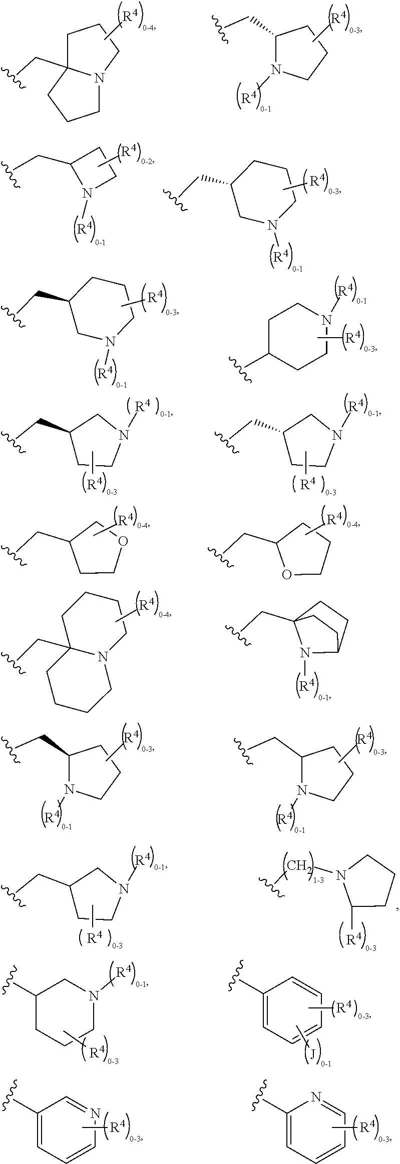

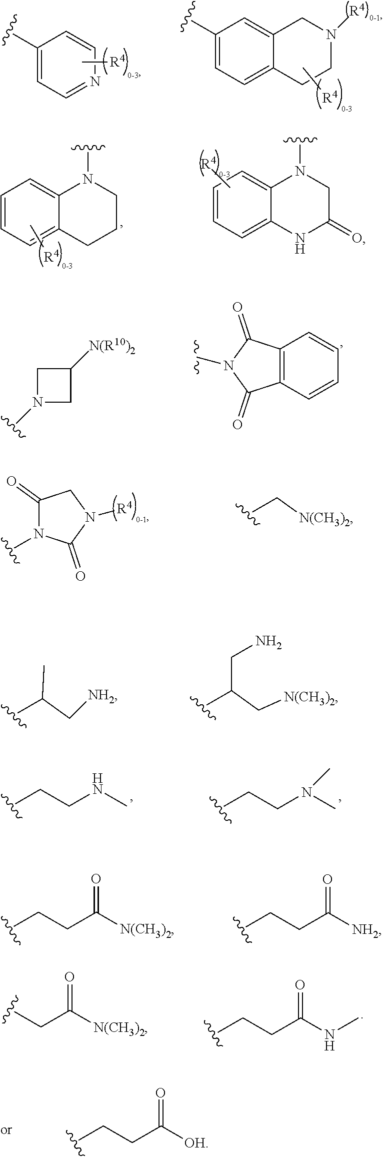

Embodiment 12(b) of this disclosure relates to the compound according to any one of Embodiments 1(b), 2(b), 3, 4, or 5, wherein R² is one of the following formulae:

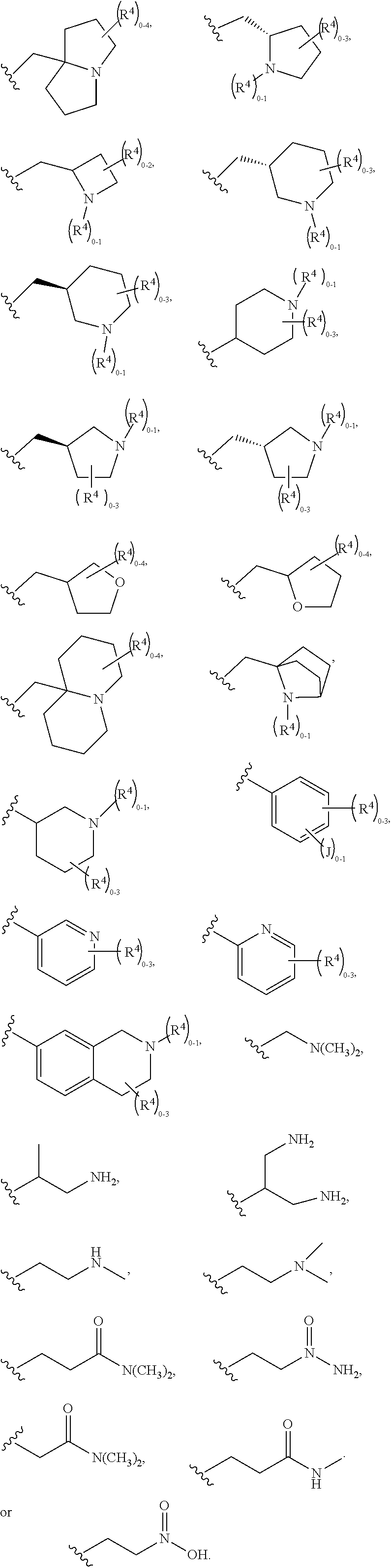

Embodiment 12(c) relates to the compound of Embodiment 12(a) wherein G is H.
Embodiment 12(d) relates to the compound of Embodiment 12(a) wherein G is not H.
Embodiment 12(e) relates to the compound of Embodiment 12(b) wherein G is H.
Embodiment 12(f) relates to the compound of Embodiment 12(b) wherein G is not H.

Embodiment 13 of this disclosure relates to the compound according to any of 1(a), 1(b), 2(a), 2(b), 3, 4, 5, 6, 7(a), 7(b), 8(a), 8(b), 9, 10(a), 10(b), 11(a), 11(b), 12(a), or 12(b), wherein R² is one of the following formulae:

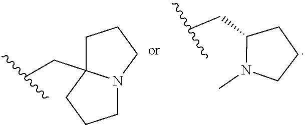

Embodiment 13(a) relates to the compound of Embodiment 13 wherein G is H.
Embodiment 13(b) relates to the compound of Embodiment 13 wherein G is not H.

Embodiment 14(a) of this disclosure relates to the compound according to any one of Embodiments 1(a), 2(a), 3, 4, 5, 6, 7(a), 8(a), 9, 10(a), 11(a), 12(a), or 13, wherein R³ is one of the following formulae:

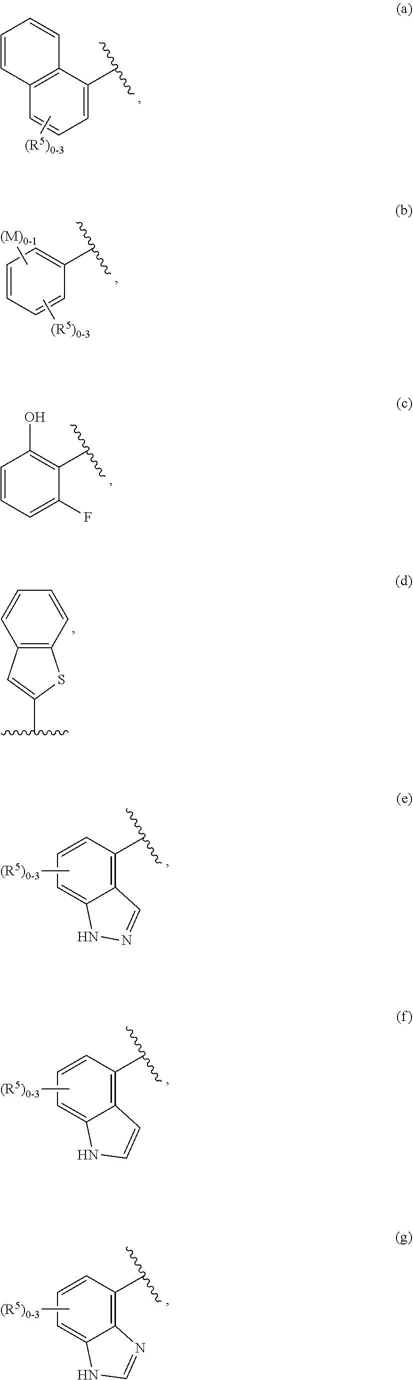

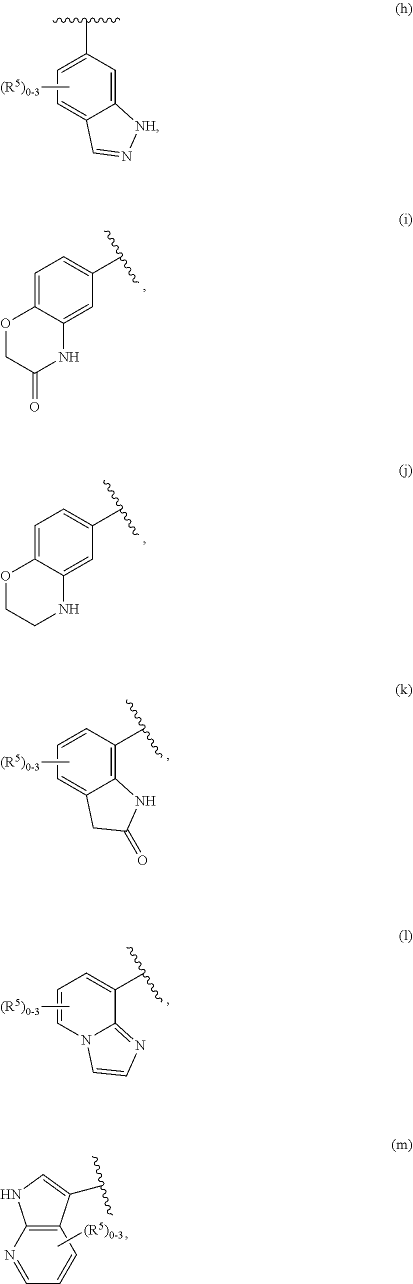

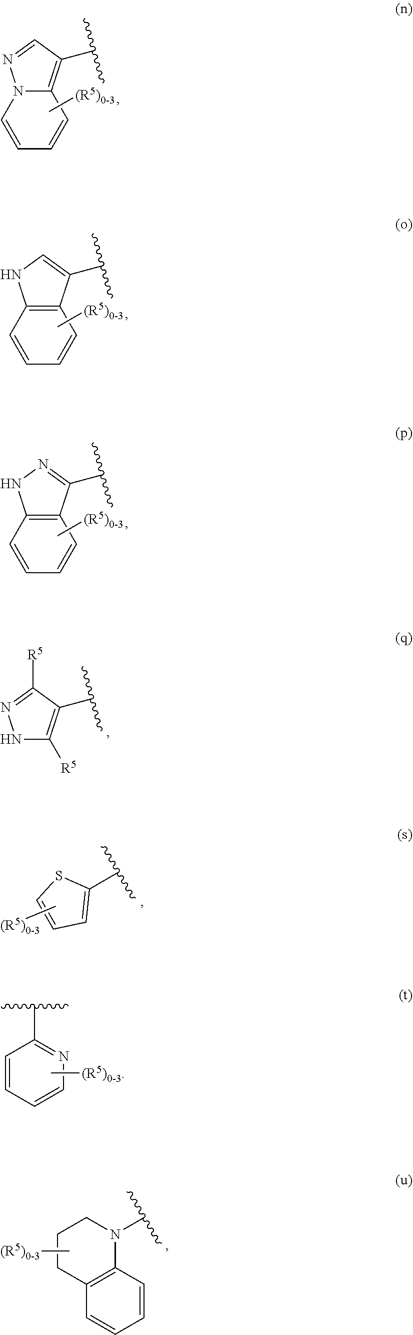

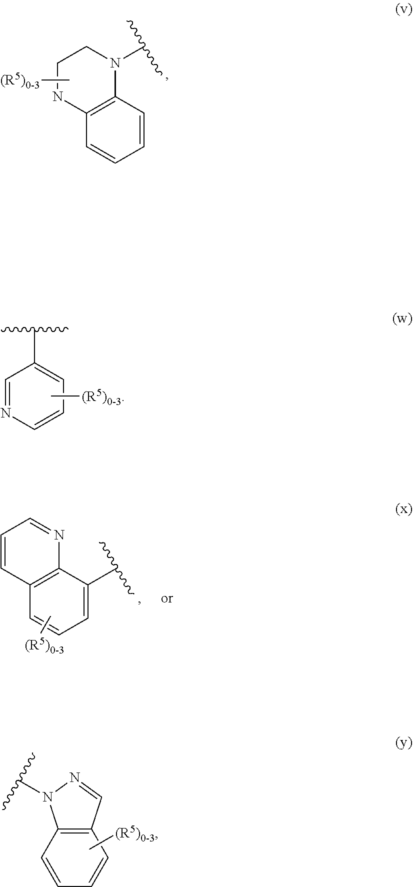

Embodiment 14(b) of this disclosure relates to the compound according to any one of Embodiments 1(b), 2(b), 3, 4, 5, 6, 7(b), 8(b), 9, 10(b), 11(b), 12(b), or 13, wherein R³ is one of the following formulae:

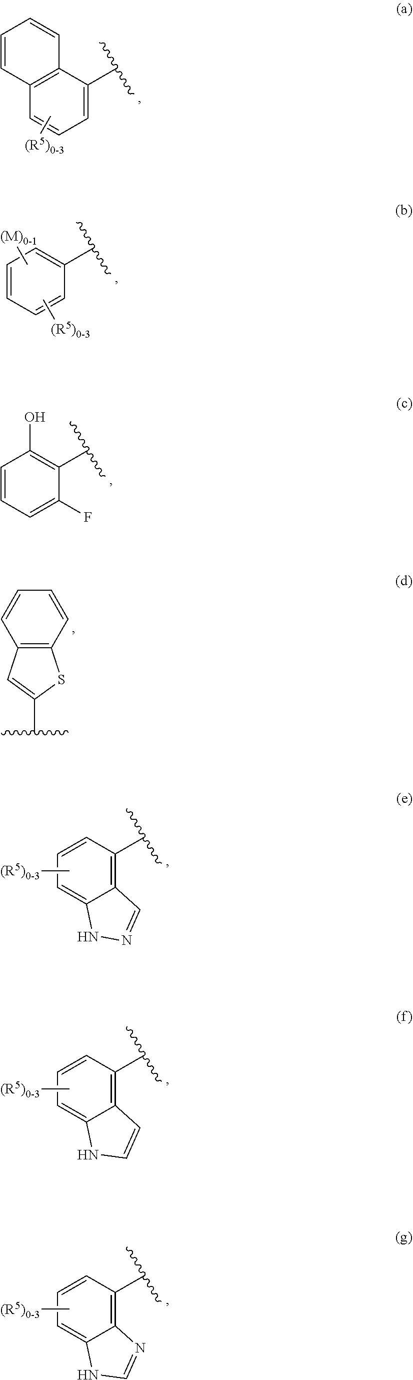

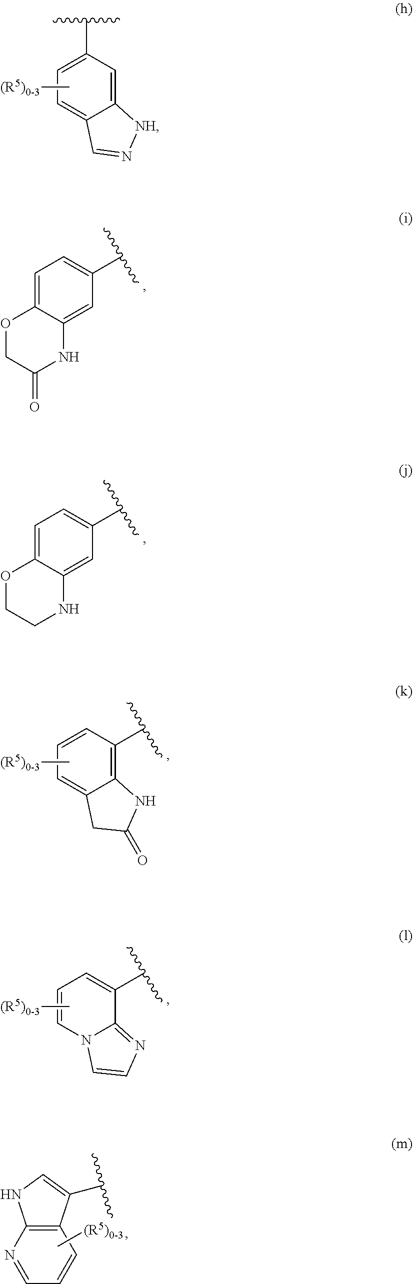

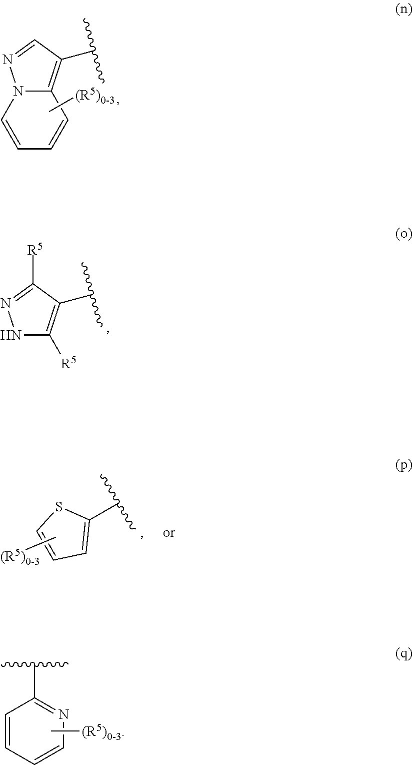

Embodiment 14(c) relates to the compound of Embodiment 14(a) wherein G is H.
Embodiment 14(d) relates to the compound of Embodiment 14(a) wherein G is not H.
Embodiment 14(e) relates to the compound of Embodiment 14(b) wherein G is H.
Embodiment 14(f) relates to the compound of Embodiment 14(b) wherein G is not H.

Embodiment 15(a) of this disclosure relates to the compound according to Embodiments 1(a), 2(a), 3, 4, 5, 6, 7(a), 8(a), 9, 10(a), 11(a), 12(a), 13, or 14(a), wherein R³ is

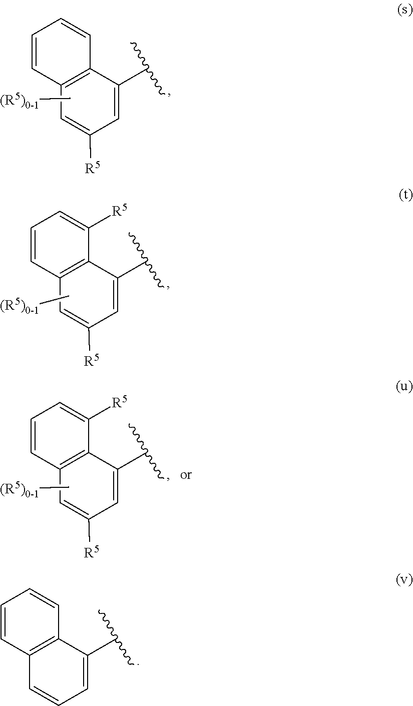

Embodiment 15(b) of this disclosure relates to the compound according to Embodiments 1(b), 2(b), 3, 4, 5, 6, 7(b), 8(b), 9, 10(b), 11(b), 12(b), 13, or 14(b), wherein R³ is

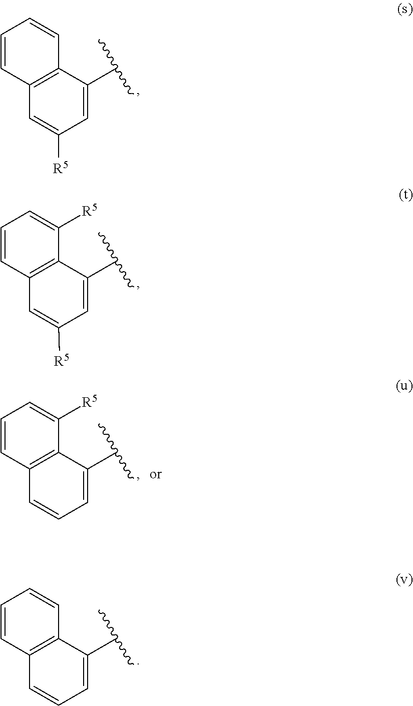

Embodiment 15(c) relates to the compound of Embodiment 15(a) wherein G is H.
Embodiment 15(d) relates to the compound of Embodiment 15(a) wherein G is not H.
Embodiment 15(e) relates to the compound of Embodiment 15(b) wherein G is H.
Embodiment 15(f) relates to the compound of Embodiment 15(b) wherein G is not H.

Embodiment 16(a) of this disclosure relates to the compound according to any of Embodiments 1(a), 2(a), 3, 4, 5, 6, 7(a), 8(a), 9, 10(a), 11(a), 12(a), 13, 14(a), or 15(a), wherein R³ is:

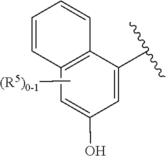

Embodiment 16(b) of this disclosure relates to the compound according to any of Embodiments 1(b), 2(b), 3, 4, 5, 6, 7(b), 8(b), 9, 10(b), 11(b), 12(b), 13, 14(b), or 15(b), wherein R³ is:

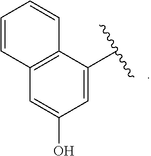 [SMILES: *c1cc(O)cc2ccccc12]

Embodiment 16(c) relates to the compound of Embodiment 16(a) wherein G is H.
Embodiment 16(d) relates to the compound of Embodiment 16(a) wherein G is not H.
Embodiment 16(e) relates to the compound of Embodiment 16(b) wherein G is H.
Embodiment 16(f) relates to the compound of Embodiment 16(b) wherein G is not H.

Embodiment 17(a) of this disclosure relates to the compound according to any of Embodiments 1(a), 2(a), 3, 4, 5, 6, 7(a), 8(a), 9, 10(a), 11(a), 12(a), 13, 14(a), 15(a) or 16(a) wherein:
L¹ is —C(O)N(H)—, —C(O)—, —C₁-C₃alkylene, —SO₂—, —SO₂N(H)—, or —C₁-C₃alkylene-C(O)—;
L² is absent or C₁-C₃alkylene; and
T² is one of the following formulae:

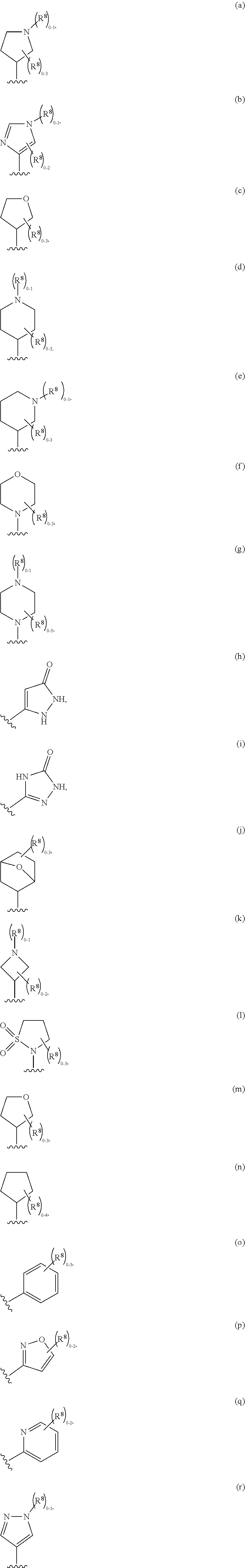

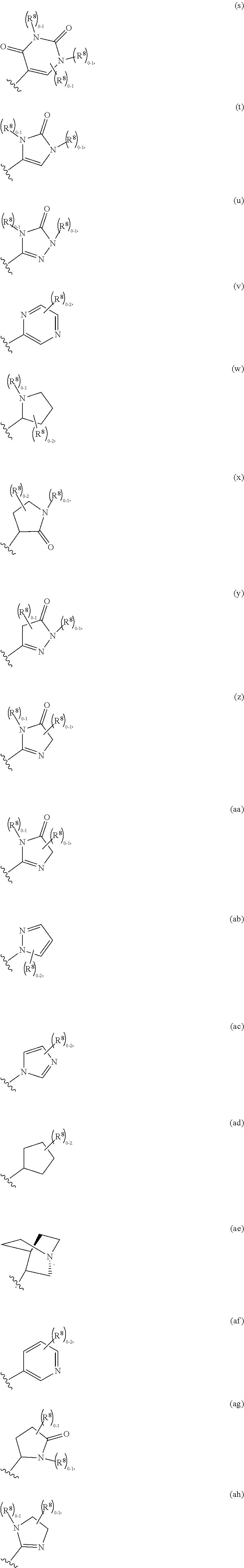

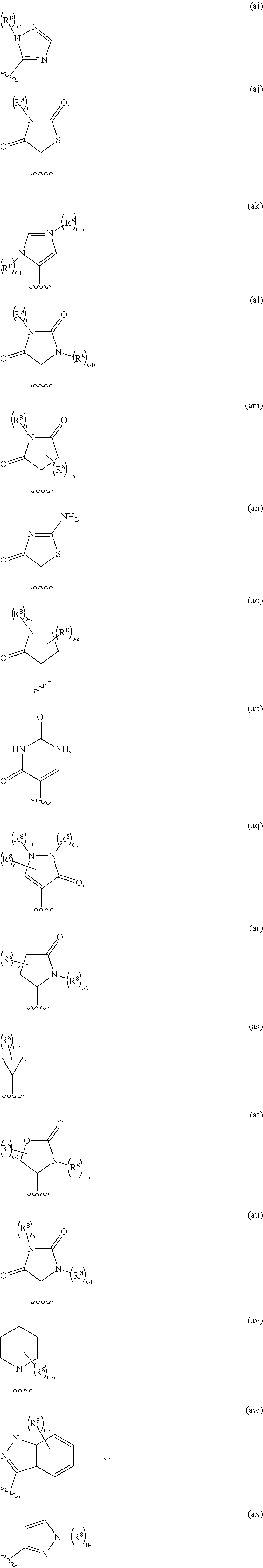

Embodiment 17(b) of this disclosure relates to the compound according to any of Embodiments 1(b), 2(b), 3, 4, 5, 6, 7(b), 8(b), 9, 10(b), 11(b), 12(b), 13, 14(b), 15(b), or 16(b) wherein:
L¹ is —C(O)N(H)—, —C(O)—, or C₁-C₃alkylene;
L² is absent or C₁-C₃alkylene; and
T² is one of the following formulae:

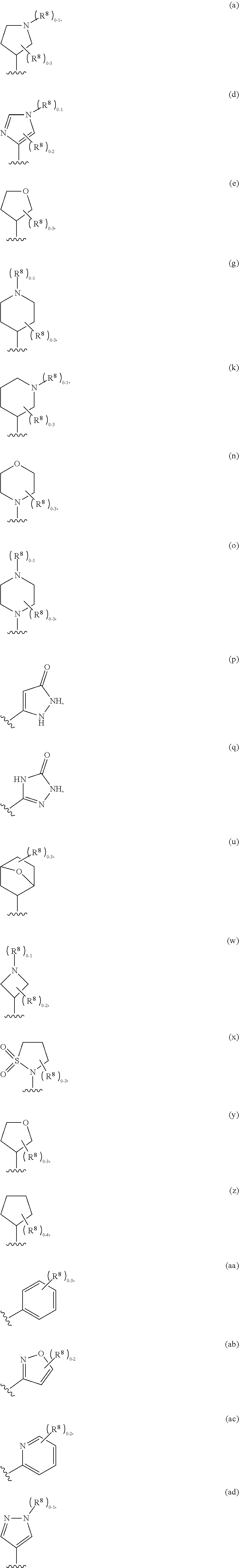

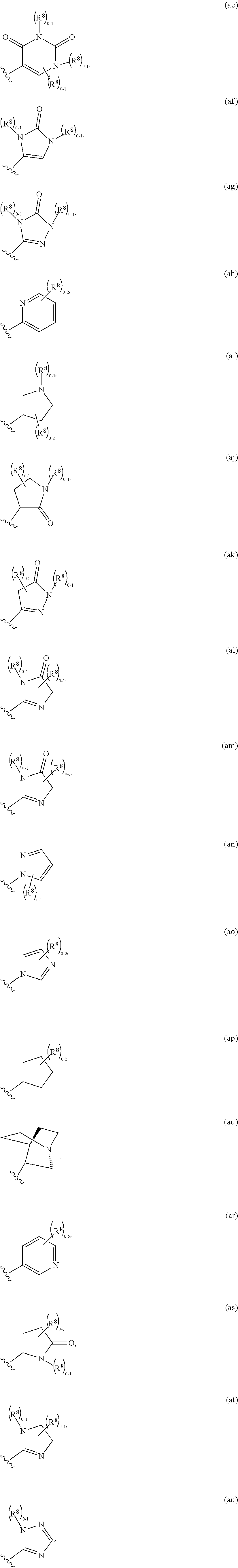

Embodiment 17(c) of this disclosure relates to the compound according to Embodiments 17(b) wherein:
L¹ is —C(O)N(H)—, —C(O)—, or C₁-C₃alkylene;
L² is absent or C₁-C₃alkylene; and
T² is one of the following formulae:

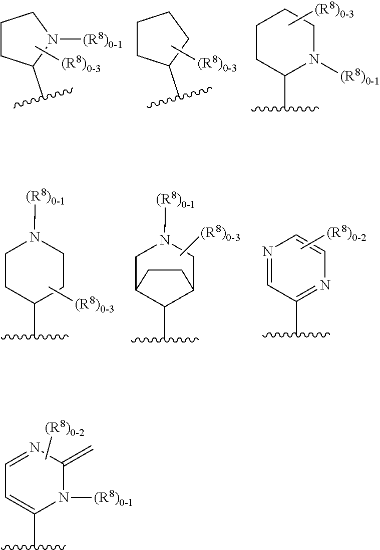

Embodiment 18(a) of this disclosure relates to the compound according to any one of Embodiments 1(a), 2(a), 3, 4, 5, 6, 7(a), 8(a), 9, 10(a), 11(a), 12(a), 13, 14(a), 15(a), 16(a), or 17(a), wherein:
L¹ is —C(O)N(H)— or —C(O)—; and
L² is absent, —CH₂—, or —CH₂—CH₂—.

Embodiment 18(b) of this disclosure relates to the compound according to any one of Embodiments 1(b), 2(b), 3, 4, 5, 6, 7(b), 8(b), 9, 10(b), 11(b), 12(b), 13, 14(b), 15(b), 16(b), or 17(b), wherein:
L¹ is —C(O)N(H)— or —C(O)—; and
L² is absent.

Embodiment 19(a) of this disclosure relates to the compound according to any one of Embodiments 1(a), 2(a), 3, 4, 5, 6, 7(a), 8(a), 9, 10(a), 11(a), 12(a), 13, 14(a), 15(a), 16(a), 17(a), or 18(a), wherein:
L¹ is absent;
L² is absent; and
T² is H.

Embodiment 19(b) of this disclosure relates to the compound according to any one of Embodiments 1(b), 2(b), 3, 4, 5, 6, 7(b), 8(b), 9, 10(b), 11(b), 12(b), 13, 14(b), 15(b), 16(b), 17(b), or 18(b), wherein:
L¹ is absent;
L² is absent; and
T² is H.

Embodiment 20(a) of this disclosure relates to the compound according to any one of Embodiments 1(a), 2(a), 3, 4, 5, 6, 7(a), 8(a), 9, 10(a), 11(a), 12(a), 13, 14(a), 15(a), or 16(a), wherein G is:
—C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, —C(O)(CH₂)₂C(O)OCH₃, or one of the following Formulae:

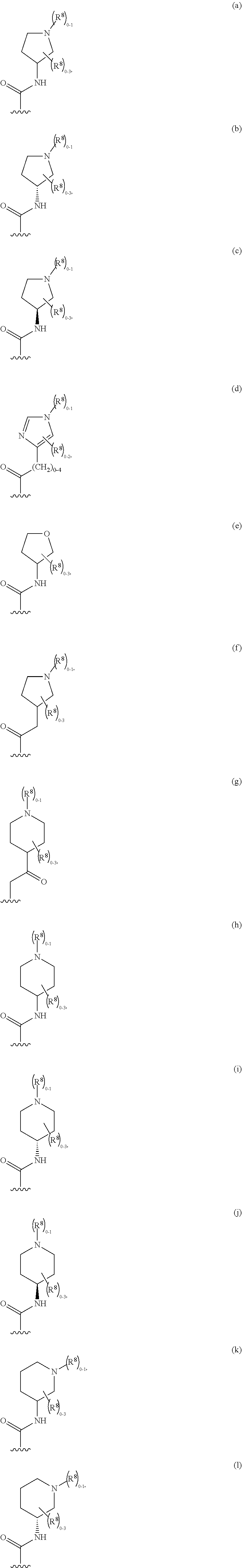

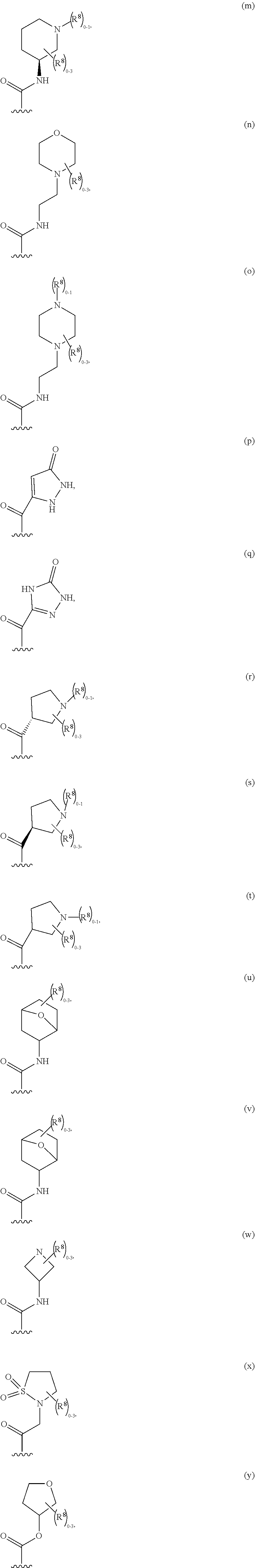

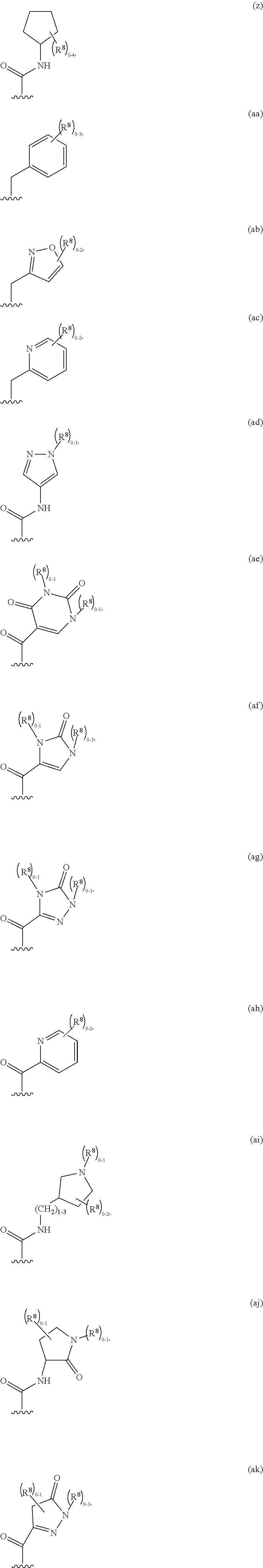

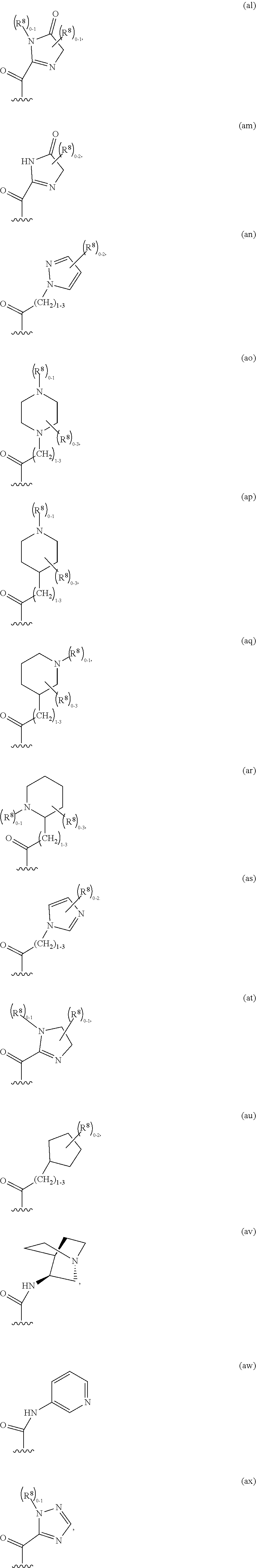

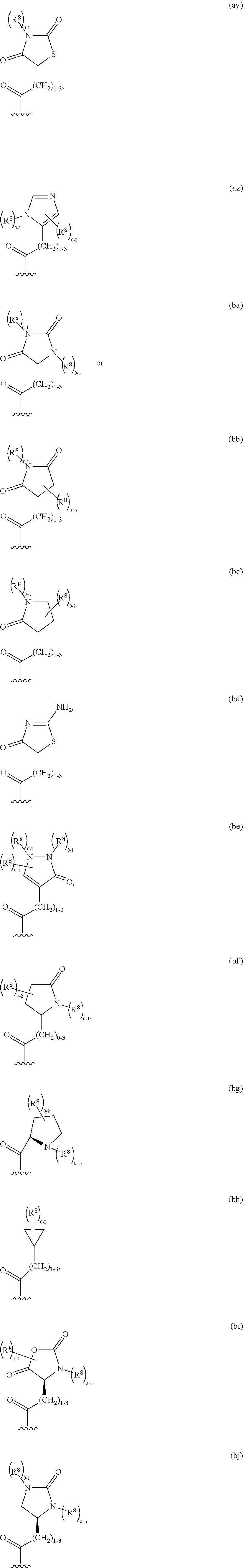

Embodiment 20(b) of this disclosure relates to the compound according to any one of Embodiments 1(b), 2(b), 3, 4, 5, 6, 7(b), 8(b), 9, 10(b), 11(b), 12(b), 13, 14(b), 15(b), or 16(b), wherein G is:
C(O)N(H)CH₂COOH, —S(O)₂(CH₂)₂OCH₃, —C(O)(CH₂)₂—SO₂—NH₂, —SO₂N(H)(CH₂)₂CH₃, —C(O)CH₂CH(OH)CH₂NH₂, —C(O)CH₂CH₂OH, —C(O)CH₂N(H)C(O)CH₃, —C(O)NH₂, —C(O)N(H)CH₂C(O)OCH₂CH₃, —C(O)CH₂NH₂, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)(CH₂)₂C(O)OH, —C(O)CH₂OC(O)CH₃, —C(O)CH₂OH, —C(O)(CH₂)₂C(O)OCH₃, or one of the following Formulae:

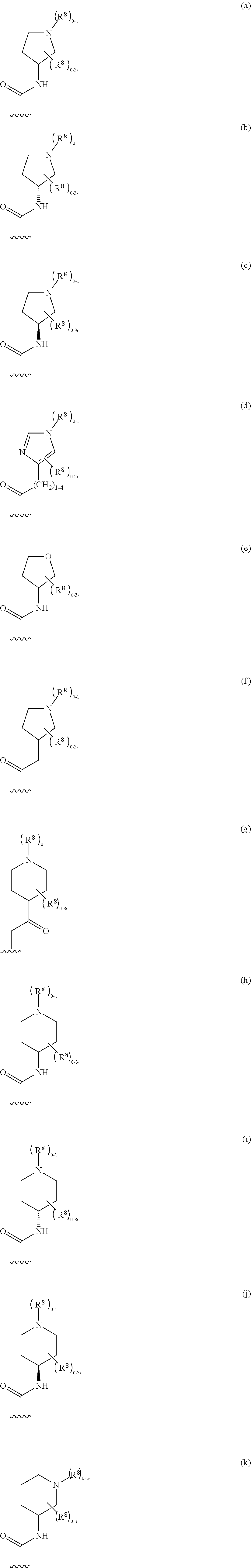

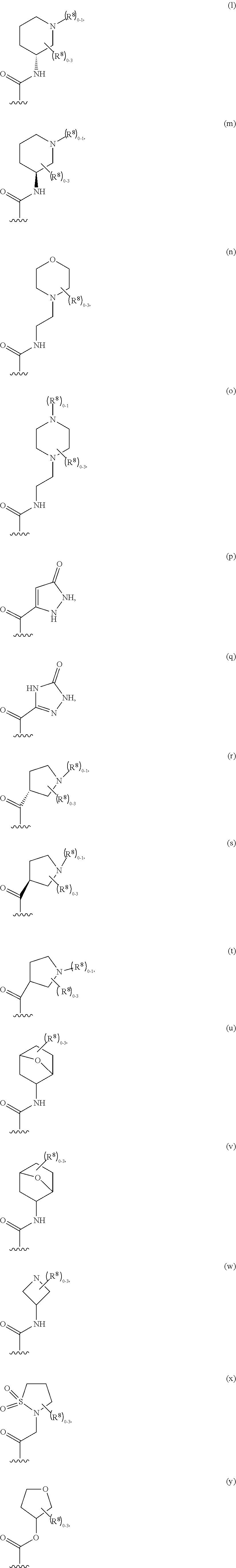

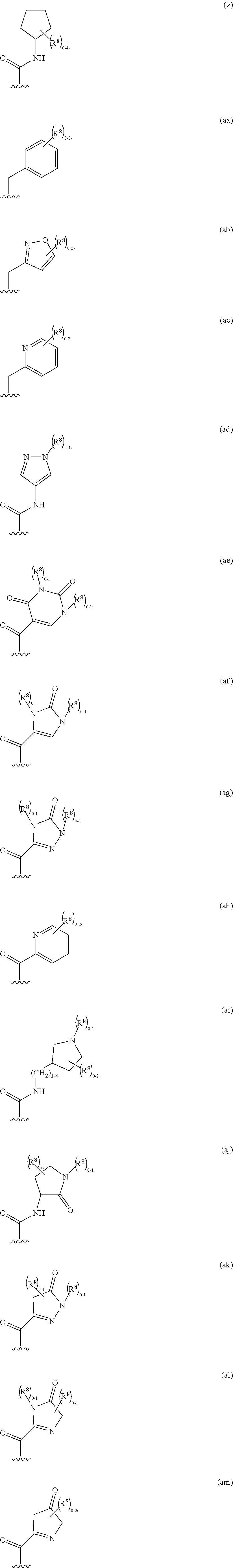

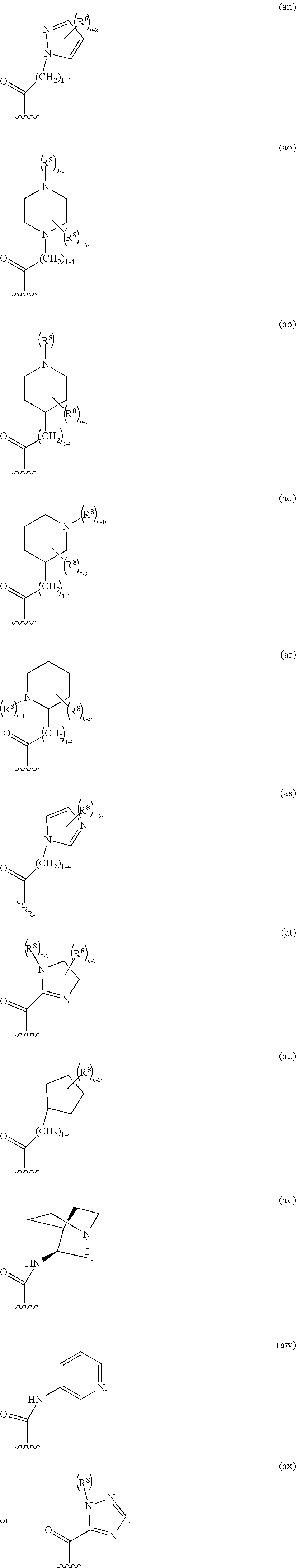

Embodiment 21(a) of this disclosure relates to the compound according to Embodiment 20(b), wherein G is —C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, or —C(O)(CH₂)₂C(O)OCH₃.

Embodiment 21(b) of this disclosure relates to the compound according to Embodiment 20(b), wherein G is —C(O)N(H)CH₂COOH, —S(O)₂(CH₂)₂OCH₃, —C(O)(CH₂)₂—SO₂—NH₂, —SO₂N(H)(CH₂)₂CH₃, —C(O)CH₂CH(OH)CH₂NH₂, —C(O)CH₂CH₂OH, —C(O)CH₂N(H)C(O)CH₃, —C(O)NH₂, —C(O)N(H)CH₂C(O)OCH₂CH₃, C(O)CH₂NH₂, —C(O)CH₂CH₃, —C(O)CH₂N(CH₃)₂, —C(O)N(H)CH₂C(CH₃)₂OH, —C(O)(CH₂)₂C(O)OH, —C(O)CH₂OC(O)CH₃, —C(O)CH₂OH or —C(O)(CH₂)₂C(O)OCH₃.

Embodiment 22(a) of this disclosure relates to the compound according to Embodiment 20(a), wherein G is one of formulae (a), (b), (c), (d), (e), (h), (i), (j), (k), (l), (m), (p), (q), (r), (s), (t), (u), (v), (w), (z), (ad), (ae), (af), (ag), (ah), (aj), (ak), (al), (am), (at), (av), (aw), (ax), or (bg).

Embodiment 22(b) of this disclosure relates to the compound according to Embodiment 20(b), wherein G is one of formulae (a), (b), (c), (d), (e), (h), (i), (j), (k), (l), (m), (p), (q), (r), (s), (t), (u), (v), (w), (z), (ad), (ae), (af), (ag), (ah), (aj), (ak), (al) or (am).

Embodiment 23(a) of this disclosure relates to the compound according to Embodiment 20(a), wherein G is one of formulae (d), (f), (n), (o), (x), (ai), (an), (ao), (ap), (aq), (ar), (as), (au), (ay), (az), (ba), (bb), (be), (bd), (be), (bf), (bi), or (bj).

Embodiment 23(b) of this disclosure relates to the compound according to Embodiment 20(b), wherein G is one of formulae (d), (f), (n), (o), (x), (ai) or (an).

Embodiment 24(a) of this disclosure relates to the compound according to any one of Embodiments 21(a), 22(a), or 23(a), wherein R² is

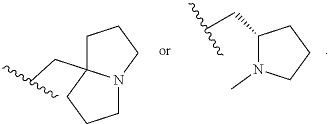

Embodiment 24(b) of this disclosure relates to the compound according to any one of Embodiments 21(b), 22(b), or 23(b), wherein R² is

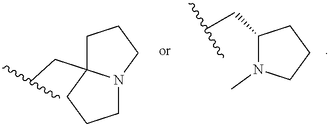

Embodiment 25 of this disclosure relates to the compound according to Embodiment 1 selected from Table IA, or a pharmaceutically acceptable salt thereof.

Embodiment 25(a) relates to the compound of Embodiment 25 wherein G is H.

Embodiment 25(b) relates to the compound of Embodiment 25 wherein G is not H.

Embodiment 1(ii) of this disclosure relates to a compound having Formula I:

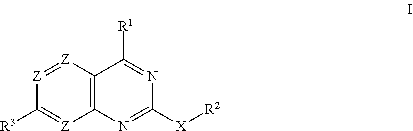

or a pharmaceutically acceptable salt, a solvate, a tautomer, a stereoisomer, or a deuterated analog thereof, wherein:
R¹ is bridged heterocycloalkyl substituted with 0-4 T¹ and 1 G;
R² is alkyl substituted with 0-5 R⁴ and 0-2 J, aryl substituted with 0-4 R⁴ and 0-1 J, arylalkyl substituted with 0-4 R⁴ and 0-1 J, heteroaryl substituted with 0-5 R⁴ and 0-1 J, heteroarylalkyl substituted with 0-5 R⁴ and 0-1 J, heterocycloalkyl substituted with 0-5 R⁴ and 0-1 J, heterocycloalkylalkyl substituted with 0-5 R⁴ and 0-1 J, bridged heterocycloalkyl substituted with 0-5 R⁴ and 0-1 J, bridged heterocycloalkylalkyl substituted with 0-5 R⁴ and 0-1 J, cycloalkyl substituted with 0-5 R⁴ and 0-1 J; cycloalkylalkyl substituted with 0-5 R⁴ and 0-1 J, or 6-11 membered heterocyclic spiro ring substituted with 0-5 R⁴ and 0-1 J; provided that when R² is alkyl substituted with 0-5 R⁴ and 0-2 J, X is not —[C(R¹⁰)₂]₁₋₆;
R³ is aryl, heteroaryl, —N(H)-heteroaryl, or heterocycloalkyl, wherein R³ is substituted with 0-4 R⁵ and 0-1 M;
each R⁴ is independently alkyl, halogen, hydroxyl, CN, haloalkyl, hydroxyalkyl, C₁-C₆cyanoalkyl, or alkoxy, provided that when R⁴ is attached to a nitrogen atom, R⁴ is not halogen or CN;
each R⁵ is independently alkyl, alkynyl, alkoxy, halogen, hydroxyl, CN, haloalkyl, C₁-C₆cyanoalkyl. C₂-C₄alkenyl, alkoxyalkyl, cycloalkyl, cycloalkylalkyl, or hydroxyalkyl, provided that when R⁵ is attached to a nitrogen atom, R⁵ is not halogen or CN;
each R⁶ is independently H, alkyl, haloalkyl, or hydroxyalkyl;
each R⁷ is independently H, halogen, alkyl, alkenyl, alkynyl, CN, haloalkyl, hydroxyalkyl, cycloalkyl, —C(O)OR¹⁰, or alkoxy optionally substituted with 1-3 halogens;
each R⁸ is independently alkyl, halogen, hydroxyl, —NH₂, —N(H)alkyl, —N(alkyl)₂, CN, SH, haloalkyl, or hydroxyalkyl, provided that when R⁸ is attached to nitrogen, R⁸ is not halogen, hydroxyl, —NH₂, —N(H)alkyl, —N(alkyl)₂, or CN;
each R⁹ is independently halogen, hydroxyl, CN, haloalkyl, or hydroxyalkyl;
each R¹⁰ is independently H, N(H)₂, alkyl, hydroxyalkyl, or haloalkyl;
R¹¹ is cycloalkyl substituted with 0-4 R⁴ and 0-1 J;
each J is independently is —N(R¹⁰)₂, —NO₂, —SO₂N(R¹⁰)₂, —NH—SO₂R¹⁰, —NHC(O)N(R¹⁰)₂, —C(O)N(R¹⁰)₂, —C(O)C(R¹⁰)₂-aryl, —C(O)OCH₂-aryl, -alkyl-N(R¹⁰)₂, -alkyl-NHC(O)alkyl, -alkyl-NHC(O)OCH₂-phenyl, —C(O)-alkylene-OH, —OC(O)R¹⁰, heterocycloalkyl substituted with 0-1 hydroxy and 0-1 halo, or —C(O)OR¹⁰; provided that when J is N(R¹⁰)₂, —NO₂, or —SO₂N(R¹⁰)₂, J is not attached to a nitrogen atom;
M is —C(O)R¹⁰, —C(O)NH₂, —N(H)C(O)CH₃, —N(H)SO₂-alkyl, NO₂, —C₁₋C₃alkyl-C(O)OR¹⁰, or —N(R¹⁰)₂, provided that M is not attached to a nitrogen atom;
X is absent, —[C(R¹⁰)₂]₁₋₆—, —[C[(R¹⁰)₂]₀₋₄—O—, —O—[C(R¹⁰)₂]₁₋₄—, C₂-C₆ alkenylene, —NR⁶—[C(R¹⁰)₂]₀₋₄—, or —[C(R¹⁰)₂]₀₋₄—NR⁶—; each Z is independently CR⁷ or N;
G is L¹-L²-T² provided that:
when Formula I is

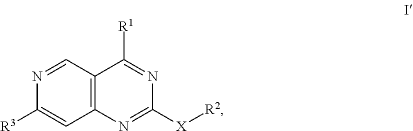

then G is not hydrogen;
each T¹ is independently alkyl, halogen, hydroxyl, haloalkyl, or hydroxyalkyl, provided that when T¹ is attached to nitrogen, T¹ is not halogen or hydroxyl;
T² is H, CN, —C(NH)N(H)CN, —C(NH)OCH₃, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)—SO₂N(R¹⁰)₂, —N(R¹⁰)—C(O)—N(R¹⁰)₂, —N(R¹⁰)—SO₂(R¹⁰), —N(R¹⁰)—C(O)O(R¹⁰), —P(O)(R¹⁰)₂, —N(R¹⁰)C(O)-alkyl, —O—C(O)-alkyl, —C(═NR¹⁰)N(R¹⁰)₂, cycloalkyl, heterocycloalkyl, bridged heterocycloalkyl, heterocyclic spiro ring, carbocyclic spiro ring, aryl, or heteroaryl, wherein T² is substituted with 0-4 R⁸ and 0-1 Rⁿ provided that when L¹ and L² are both absent, T² is not CN, —N(R¹⁰)—SO₂N(R¹⁰)₂, —N(R¹⁰)—C(O)—N(R¹⁰)₂, N(R¹⁰)₂, —OR¹⁰, —N(R¹⁰)C(O)-alkyl, or —O—C(O)-alkyl, and provided that when L¹ and L² taken together are —C(O), —C(O)C(O)N(H)—, —C(O)O—, —C(O)N(H)—, —S(O)₂—, -alkylene-C(O)N(H)—, -alkylene-C(O)—, —SO₂—, or —SO₂NH—, T² is not CN, —N(R¹⁰)—SO₂N(R¹⁰)₂, —N(R¹⁰)—C(O)—N(R¹⁰)₂, —C(O)OR¹⁰, —N(R¹⁰)₂, —OR¹⁰, —C(O)N(R¹⁰)₂, —SO₂N(R¹⁰)₂, —N(R¹⁰)C(O)-alkyl, —O—C(O)-alkyl, —C(NH)OCH₃, or —C(═NR¹⁰)N(R¹⁰)₂;
L¹ is absent, —C(O)—, —C(O)C(O)N(H)—, —C(O)O—, —C(O)N(H)—, —C(O)N(H)—O—, -alkylene-C(O)N(H)—, -alkylene-C(O)—, —SO₂—, or —SO₂N(H)—, provided that when L¹ is —C(O)N(H)—O—, T² is H; and
L² is absent, alkylene substituted with 0-4 R⁹, —C₂-C₄alkenylene, or C₃-C₆alkenylene substituted with 0-4 R⁹, provided that when L¹ is —C(O)— and L² is absent, T² is not H.
Embodiment 1(ii)(a) relates to Embodiment 1(ii) wherein G is H.
Embodiment 1(ii)(b) relates to Embodiment 1(ii) wherein G is not H.
Embodiment 1 (ii)(c) relates to Embodiment 1(ii) or 1 (ii)(b) wherein G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 1(ii)(d) relates to Embodiment 1(ii) wherein G has one of the following Formulae:

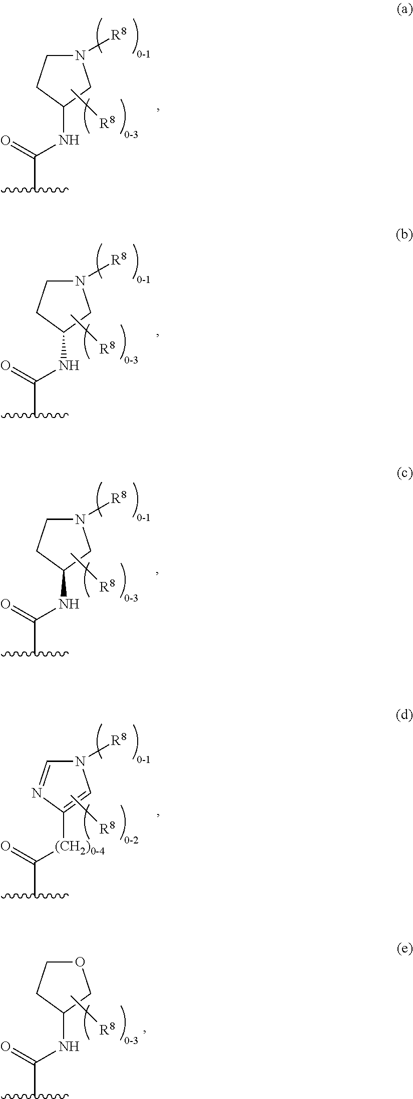

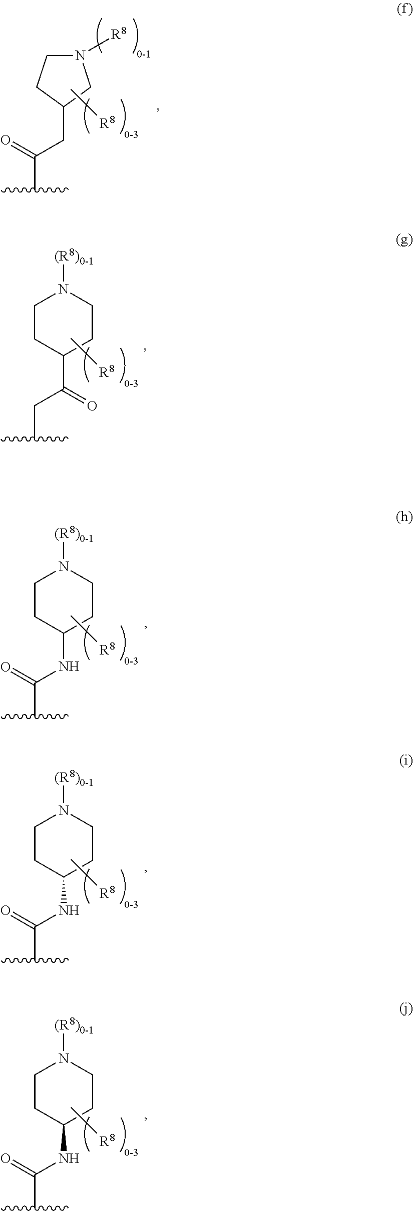

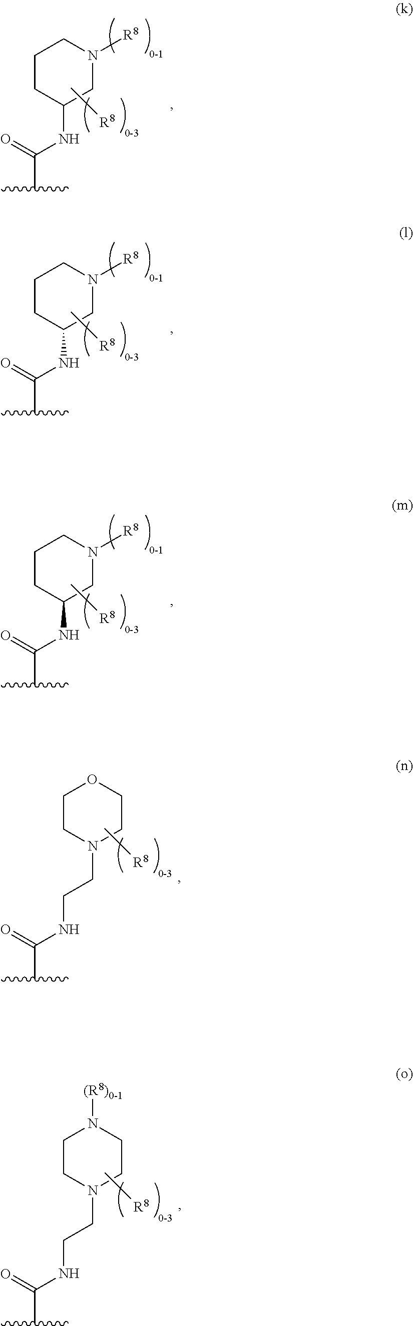

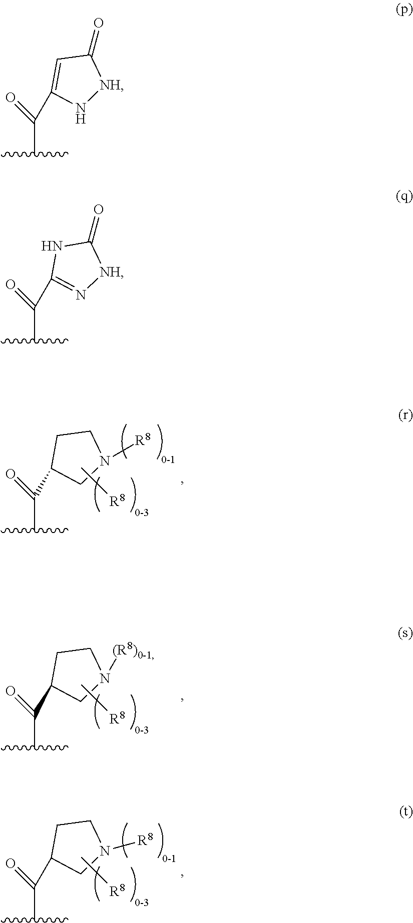

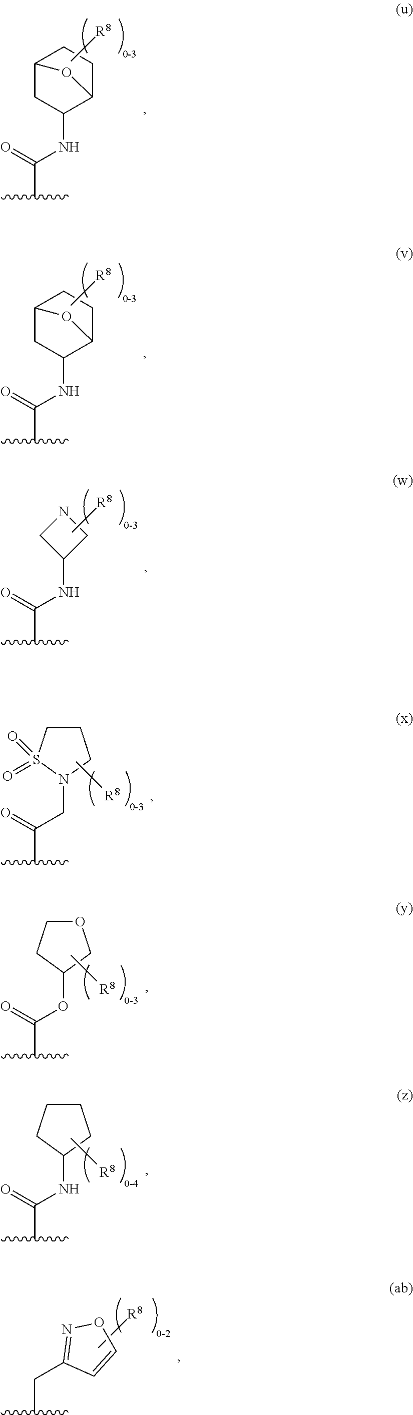

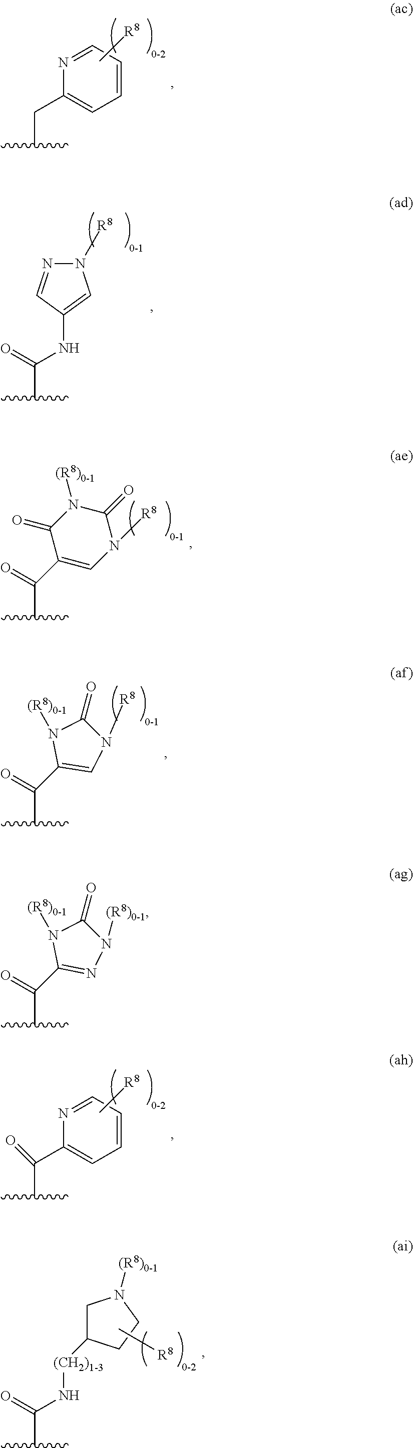

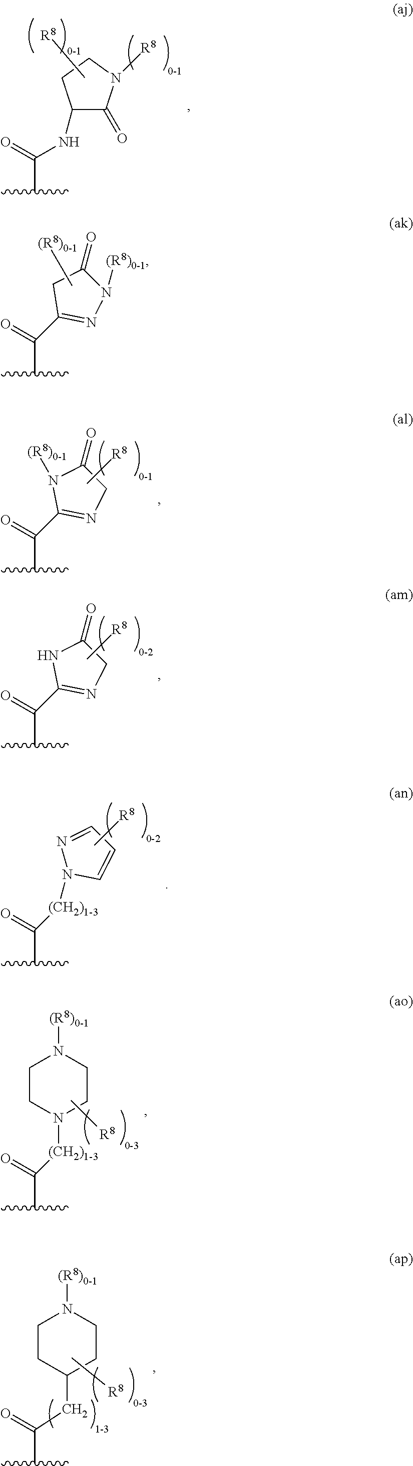

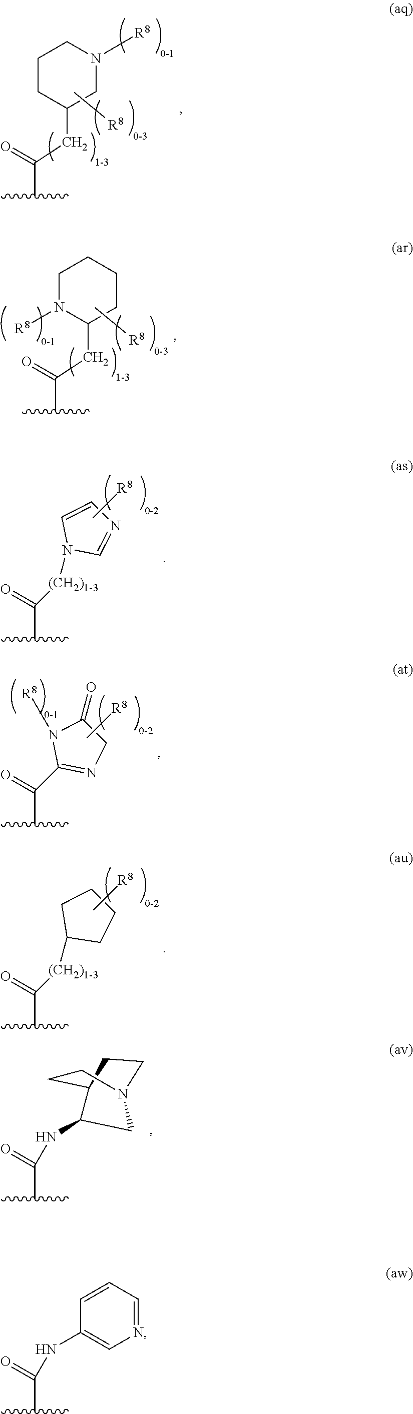

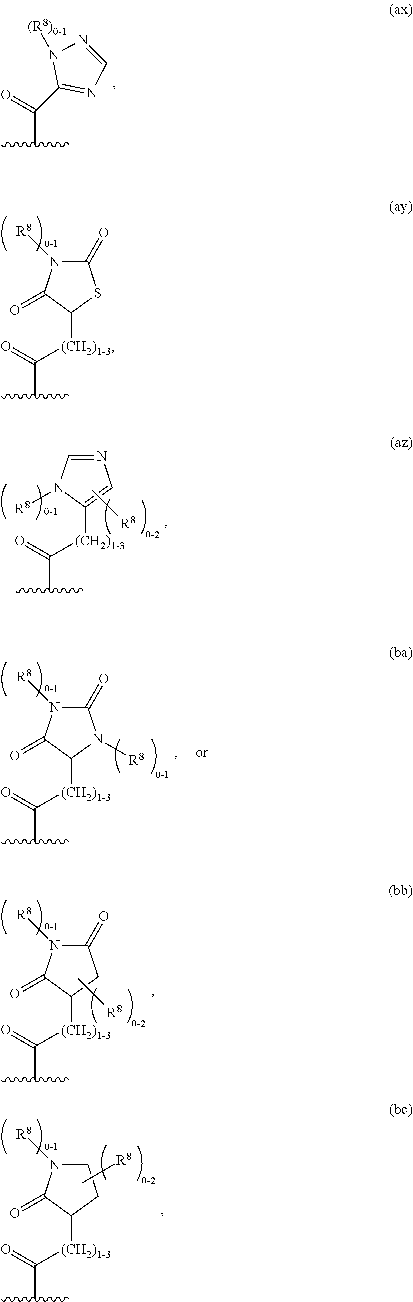

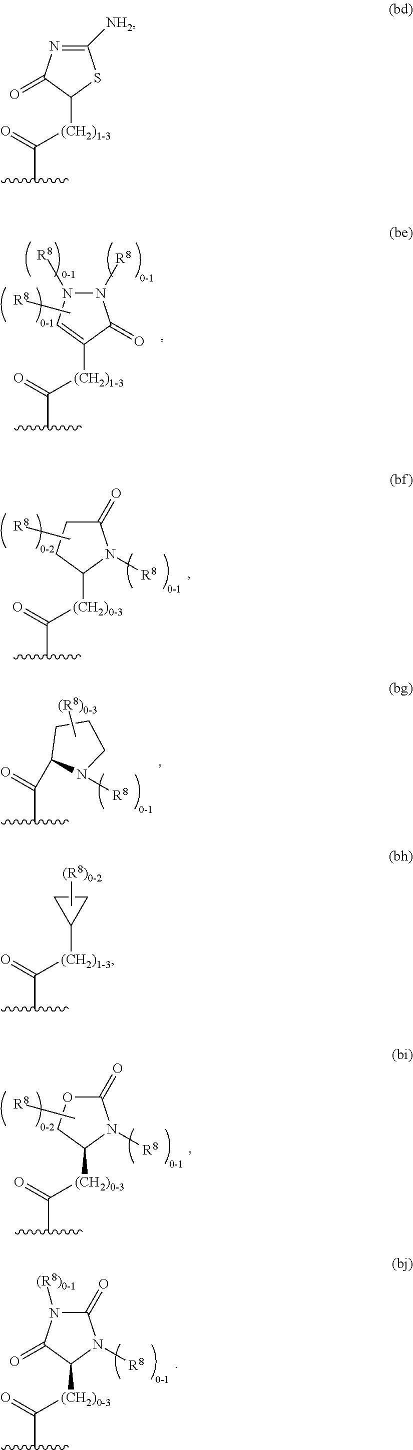

Embodiment 1 (ii)(e) relates to Embodiment 1(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 1(iii) relates to any one of Embodiments 1(ii), 1(ii)(a), 1(ii)(b), 1(ii)(c), 1(ii)(d), or 1(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 1(iv) relates to any one of Embodiments 1(ii), 1(ii)(a), 1(ii)(b), 1(ii)(c), 1(ii)(d), or 1(ii)(e) wherein R² is saturated 7-9 membered heterocycloalkyl comprising at least one nitrogen.

Embodiment 1(v) relates to any one of Embodiments 1(ii), 1(ii)(a), 1(ii)(b), 1(ii)(c), 1(ii)(d), or 1(ii)(e) wherein R² is:

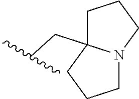 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 1(vi) relates to Embodiment 1(ii) wherein each Z is CR⁷.

Embodiment 1(vii) relates to Embodiment 1(ii) wherein when G is H, then R² is

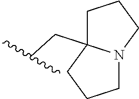 [SMILES: *CC12CCCN1CCC2]

that is substituted with 0-3 R⁴.

Embodiment 2(ii) of this disclosure relates to the compound of Embodiment 1(ii), wherein:
X is absent, —[C(R¹⁰)₂]₁₋₄—, —(CH₂)₀₋₃—O—, O—[C(R¹⁰)₂]₁₋₃—, C₃-C₄ alkenylene, or —NR⁶—
R¹ is a 6-9 membered bridged heterocycloalkyl having at least one nitrogen atom, wherein R¹ is substituted with 0-4 T¹ and 1 G;
R² is C₁-C₆alkyl substituted with 0-5 R⁴ and 0-2 J, phenyl substituted with 0-4 R⁴ and 0-1 J, —C₀-C₆alkyl-phenyl substituted with 0-4 R⁴ and 0-1 J, 5-10 membered heteroaryl substituted with 0-5 R⁴ and 0-1 J, —C₀-C₆alkyl-5-10 membered heteroaryl substituted with 0-5 R⁴ and 0-1 J, —C₀-C₆alkyl-4-10 membered heterocycloalkyl substituted with 0-5 R⁴ and 0-1 J, or —C₀-C₆alkyl-C₃-C₁₀cycloalkyl substituted with 0-5 R⁴ and 0-1 J, provided that when R² is C₁-C₆alkyl substituted with 0-5 R⁴ and 0-2 J, X is not-C[(R¹⁰)₂]₁₋₃;
R³ is phenyl, naphthyl, 5-10 membered heteroaryl, or 5-10 membered heterocycloalkyl, wherein R³ is substituted with 0-4 R⁵ and 0-1 M;
each R⁴ is independently C₁-C₆alkyl, halogen, hydroxyl, CN, C₁-C₆haloalkyl, C₁-C₆hydroxyalkyl, C₁-C₆cyanoalkyl or C₁-C₆alkoxy, provided that when R⁴ is attached to a nitrogen atom, R⁴ is not halogen, or CN;
each R⁵ is independently C₁-C₆alkyl, C₁-C₆alkynyl, C₁-C₆alkoxy, halogen, hydroxyl, CN, C₁-C₆haloalkyl, C₁-C₆cynoalkyl, or C₁-C₆hydroxyalkyl, provided that when R⁵ is attached to a nitrogen atom, R⁵ is not halogen or CN;
each R⁶ is independently H, C₁-C₆alkyl, C₁-C₆haloalkyl, or C₁-C₆hydroxyalkyl; each R⁷ is independently H, halogen, C₁-C₄alkyl, C₂-C₄alkenyl, C₂-C₄alkynyl, CN, C₁-C₄haloalkyl, C₁-C₄hydroxyalkyl; C₃-C₄cycloalkyl, —C(O)OR¹⁰, or C₁-C₄alkoxy optionally substituted with 1-3 halogens;
each R⁸ is independently C₁-C₆alkyl, halogen, hydroxyl, —NH₂, —N(H)C₁-C₆alkyl, —N(C₁-C₆alkyl)₂, CN, C₁-C₆haloalkyl, or C₁-C₆hydroxyalkyl, provided that when R⁸ is attached to nitrogen, R⁸ is not halogen, hydroxyl, —NH₂, —N(H)C₁-C₆alkyl, —N(C₁-C₆alkyl)₂, or CN;
each R⁹ is independently halogen, hydroxyl, CN, C₁-C₆haloalkyl, or C₁-C₆hydroxyalkyl;
each R¹⁰ is independently H, —N(H)₂, C₁-C₆alkyl, or C₁-C₆1haloalkyl;
R¹¹ is C₃-C₆cycloalkyl substituted with 0-3 R⁴ and 0-1 J;
each J is independently is —N(R¹⁰)₂, —SO₂N(R¹⁰)₂, —C(O)N(R¹⁰)₂, or —C(O)OR¹⁰; provided that when J is —N(R¹⁰)₂ or —SO₂N(R¹⁰)₂, J is not attached to a nitrogen atom;
each T¹ is independently C₁-C₆alkyl halogen, hydroxyl, C₁-C₆haloalkyl, or C₁-C₆hydroxyalkyl, provided that when T¹ is attached to nitrogen, T¹ is not halogen or hydroxyl;
T² is H, CN, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)C(O)—C₁-C₆alkyl, —O—C(O)—C₁-C₆alkyl, —C(═NH)NH₂, C₃-C₆cycloalkyl, 4-10 membered heterocycloalkyl, 5-9 membered bridged heterocycloalkyl, 6-9 membered heterocyclic spiro ring, 6-9 membered carbocyclic spiro ring, phenyl, naphthyl, or 5-10 membered heteroaryl, wherein T² is substituted with 0-4 R⁸ and 0-1 R¹¹;
provided that when L¹ and L² are both absent, T² is not CN, —N(R¹⁰)₂, —OR¹⁰, —N(R¹⁰)C(O)—C₁-C₆alkyl, or —O—C(O)—C₁-C₆alkyl, and provided that when L¹ and L² taken together are —C(O)—, —C(O)C(O)N(H)—, —C(O)O—, —C(O)N(H)—, —C(O)N(H)—O—, —C₁-C₆alkyelene-C(O)N(H)—, —C₁-C₆ alkelene-C(O)—, —SO₂—, or —SO₂NH—, T² is not CN, —C(O)OR¹⁰, C₁-C₅alkoxy, N(R¹⁰)₂, —OR¹⁰, —C(O)N(R¹⁰)₂, —SO₂N(R¹⁰)₂, —N(R¹⁰)C(O)—C₁-C₆alkyl, —O—C(O)— C₁-C₆alkyl or —C(═NH)NH₂;
L¹ is absent, —C(O)—, —C(O)C(O)N(H)—, —C(O)O—, —C(O)N(H)—, —C₁-C₆alkyelene-C(O)N(H)—, —C₁-C₆alkylene-C(O)—, —SO₂—, or —SO₂N(H)— provided that when L¹ is —C(O)N(H)—O—, T² is H;
L² is absent, C₁-C₆alkylene substituted with 0-2 R⁹, or C₃-C₆alkenylene substituted with 0-4 R⁹, provided that when L¹ is —C(O)— and L² is absent, T² is not H; and
M is —C(O)NH₂, —NH₂C(O)CH₃, —N(H)SO₂— C₁-C₃alkyl, or —NH₂, provided that M is not attached to a nitrogen atom.
Embodiment 2(ii)(a) relates to Embodiment 2(ii) wherein G is H.
Embodiment 2(ii)(b) relates to Embodiment 2(ii) wherein G is not H.
Embodiment 2(ii)(c) relates to Embodiment 2(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 2(iii) relates to any one of Embodiments 2(ii), 2(ii)(a), 2(ii)(b), or 2(ii)(c) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 2(iv) relates to any one of Embodiments 2(ii), 2(ii)(a), 2(ii)(b), or 2(ii)(c) wherein R² is saturated 7-9 membered heterocycloalkyl comprising at least one nitrogen.

Embodiment 2(v) relates to any one of Embodiments 2(ii), 2(ii)(a), 2(ii)(b), or 2(ii)(c) wherein R² is

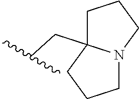 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 2(vi) relates to Embodiment 2(ii) wherein each Z is CR⁷.

Embodiment 2(vii) relates to Embodiment 2(ii) wherein when G is H, then R² is

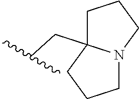 [SMILES: *CC12CCCN1CCC2]

that is substituted with 0-3 R⁴.

Embodiment 3(ii) of this disclosure relates to the compound of any one of Embodiments 1(ii), 1(ii)(a), 1(ii)(b), 1(ii)(c), 1(ii)(d), 1(ii)(e), 2(ii), 2(ii)(a), 2(ii)(b), or 2(ii)(c) having one of the following formulae:

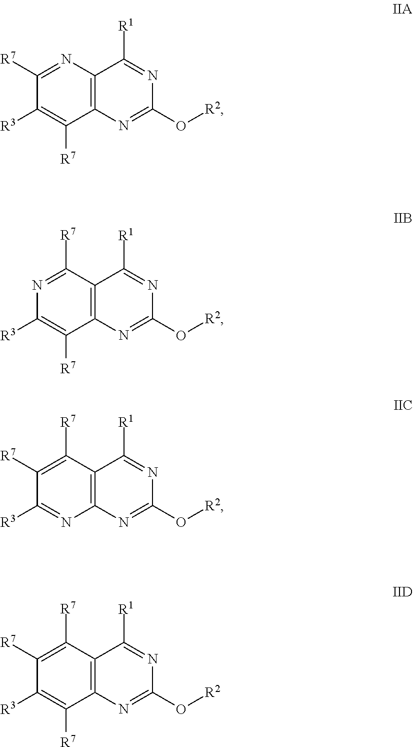

or a pharmaceutically acceptable salt, a solvate, a tautomer, a stereoisomer, or a deuterated analog thereof.
Embodiment 3(ii)(a) relates to Embodiment 3(ii) wherein G is H, provided that when the compound has the Formula (IIB), R⁷ is not H.
Embodiment 3(ii)(b) relates to Embodiment 3(ii) wherein G is not H.
Embodiment 3(ii)(c) relates to Embodiment 3(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 3(ii)(d) relates to Embodiment 3(ii) wherein G has one of the following Formulae:

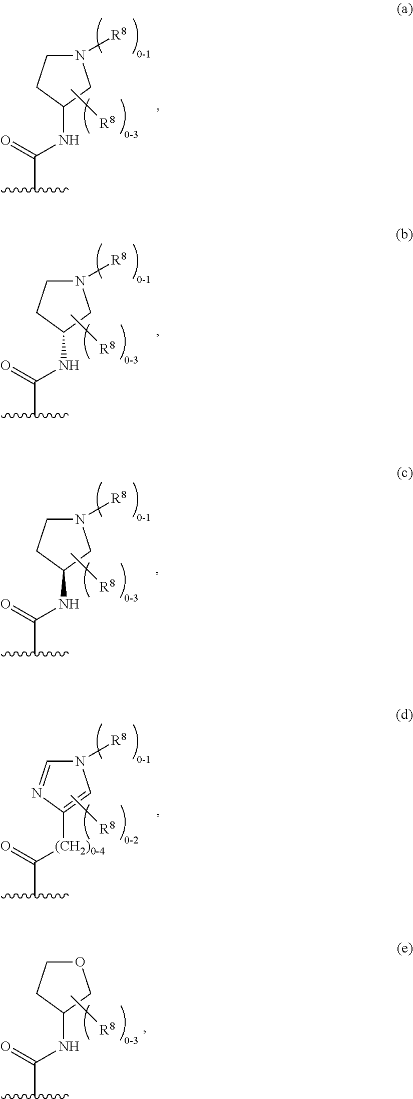

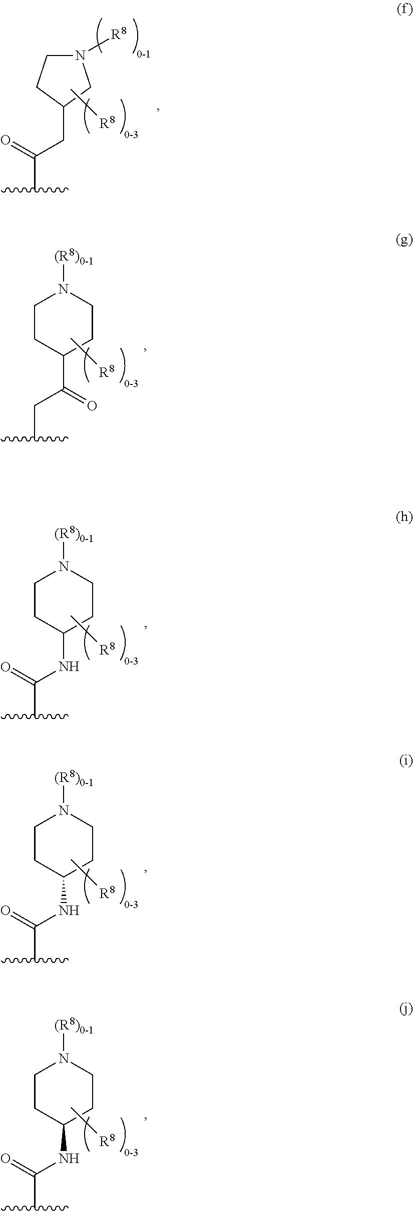

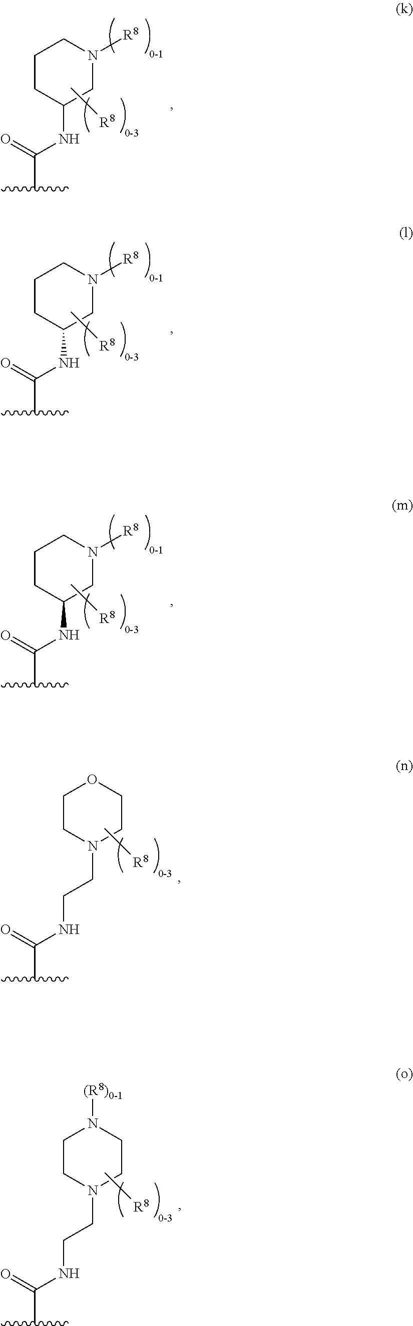

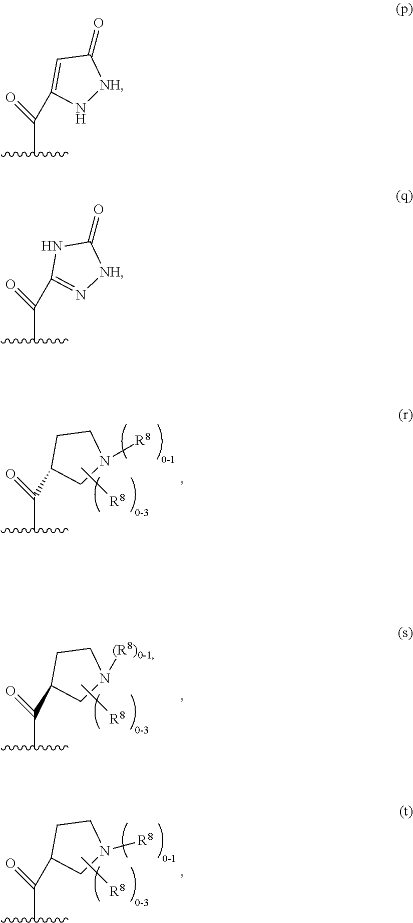

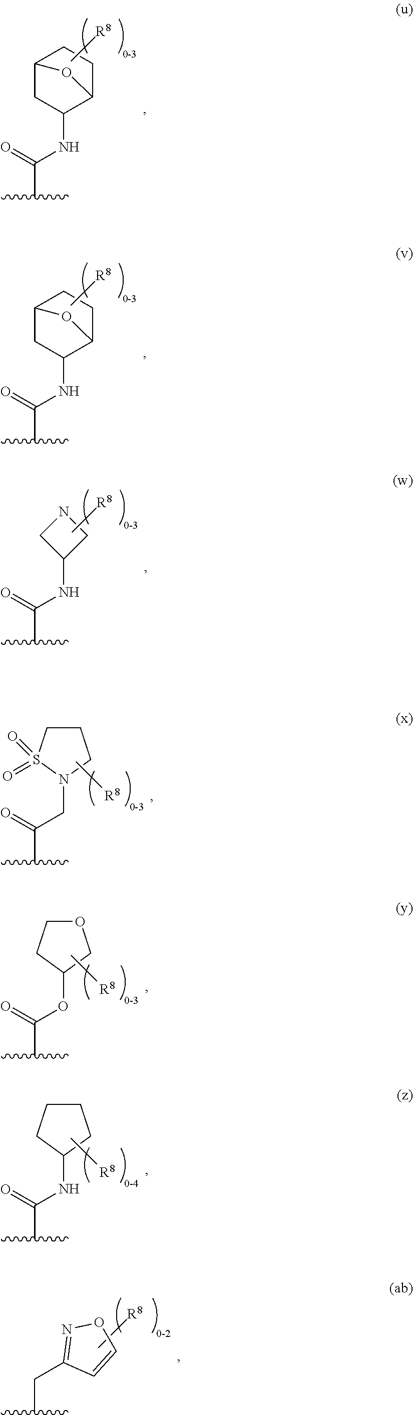

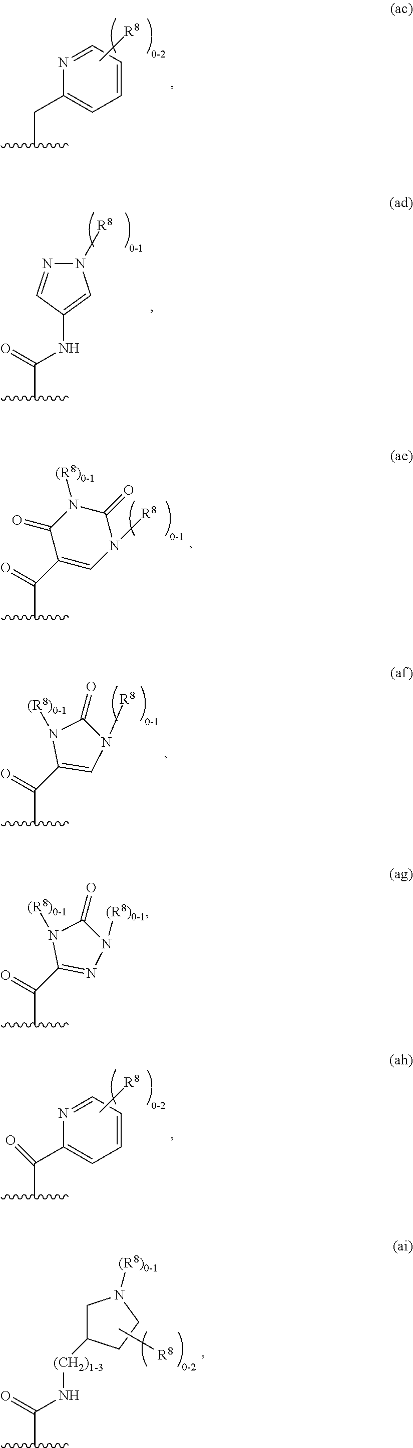

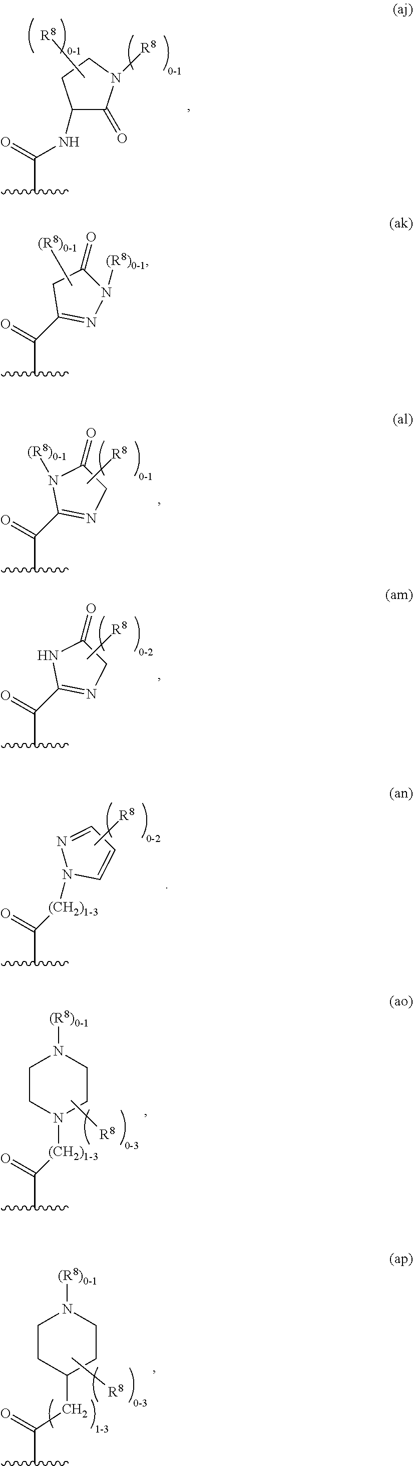

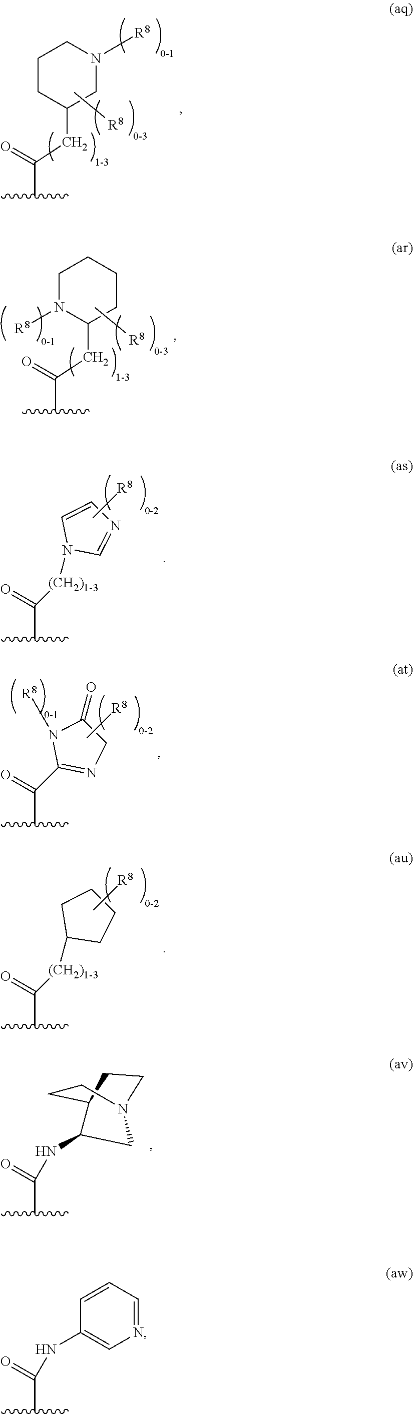

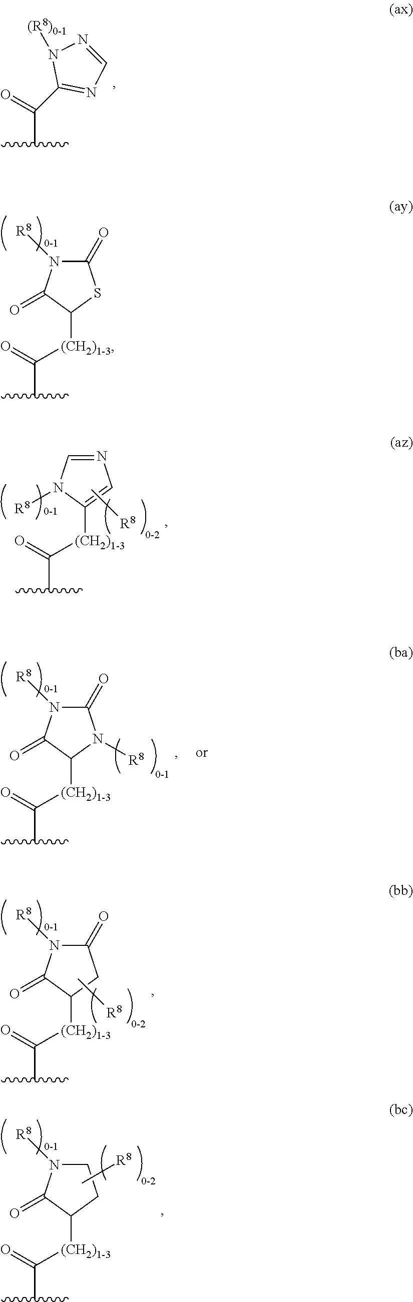

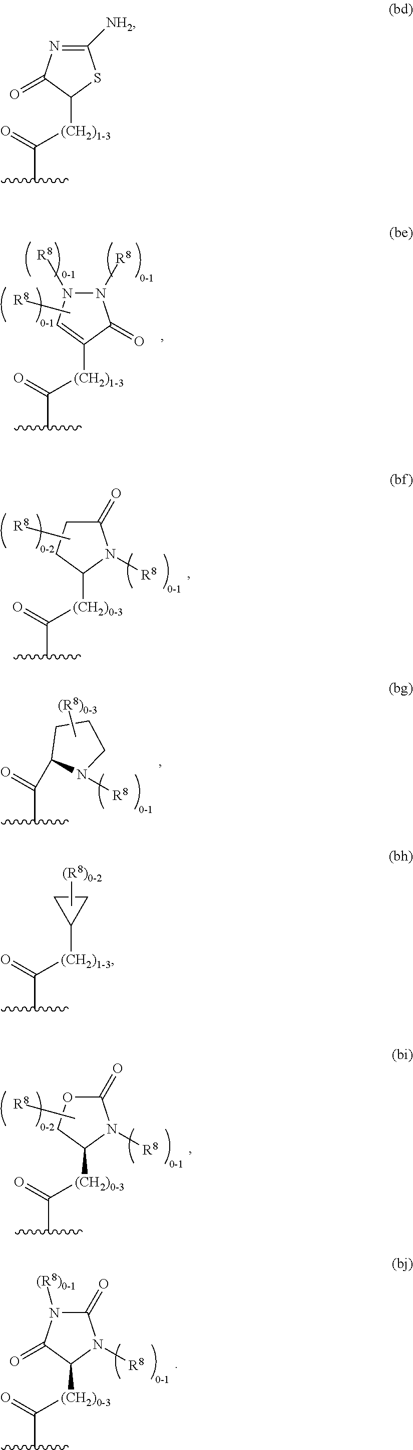

Embodiment 3(ii)(e) relates to Embodiment 3(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 3(iii) relates to any one of Embodiments 3(ii), 3(ii)(a), 3(ii)(b), 3(ii)(c), 3(ii)(d), or 3(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 3(iv) relates to any one of Embodiments 3(ii), 3(ii)(a), 3(ii)(b), 3(ii)(c), 3(ii)(d), or 3(ii)(e) wherein R² is saturated 7-9 membered heterocycloalkyl comprising at least one nitrogen.

Embodiment 3(v) relates to any one of Embodiments 3(ii), 3(ii)(a), 3(ii)(b), 3(ii)(c), 3(ii)(d), or 3(ii)(e) wherein R² is

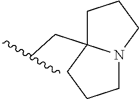 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 3(vi) relates to Embodiment 3(ii) wherein when G is H, then R² is

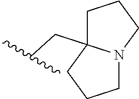 [SMILES: *CC12CCCN1CCC2]

that is substituted with 0-3 R⁴.

Embodiment 4(ii) of this disclosure relates to the compound of Embodiment 3(ii) having Formula IIB or IID, or a pharmaceutically acceptable salt, a solvate, a tautomer, a stereoisomer, or a deuterated analog thereof.
Embodiment 4(ii)(a) relates to Embodiment 4(ii) wherein G is H, provided that when the compound has the Formula (IIB), R⁷ is not H.
Embodiment 4(ii)(b) relates to Embodiment 4(ii) wherein G is not H.
Embodiment 4(ii)(c) relates to Embodiment 4(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 4(ii)(d) relates to Embodiment 4(ii) wherein G has one of the following Formulae:

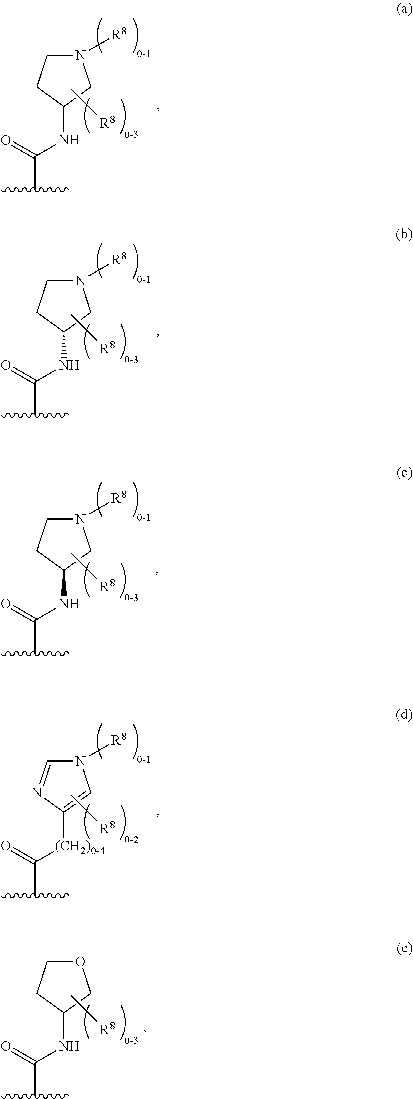

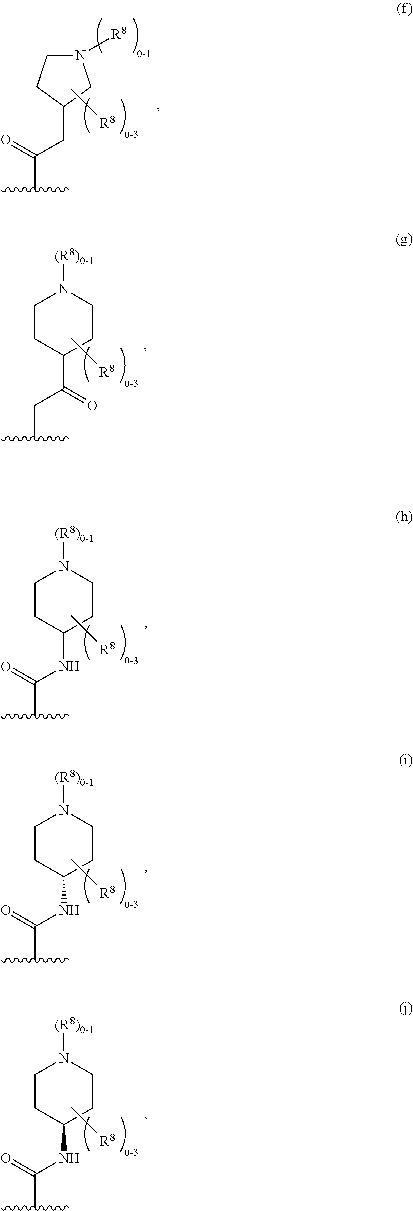

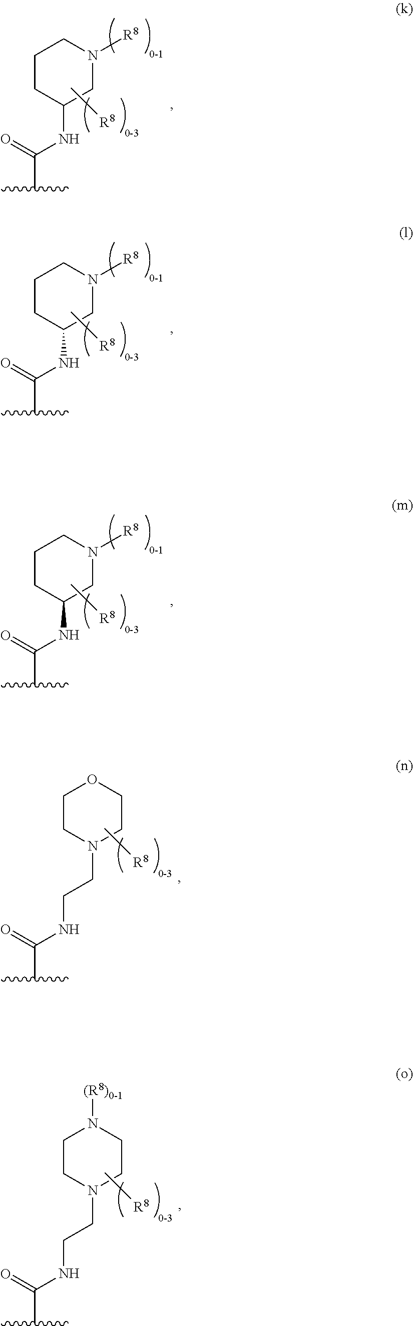

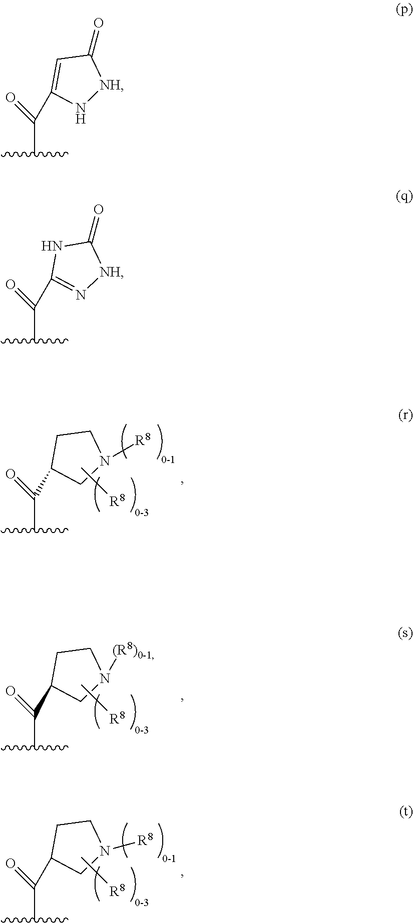

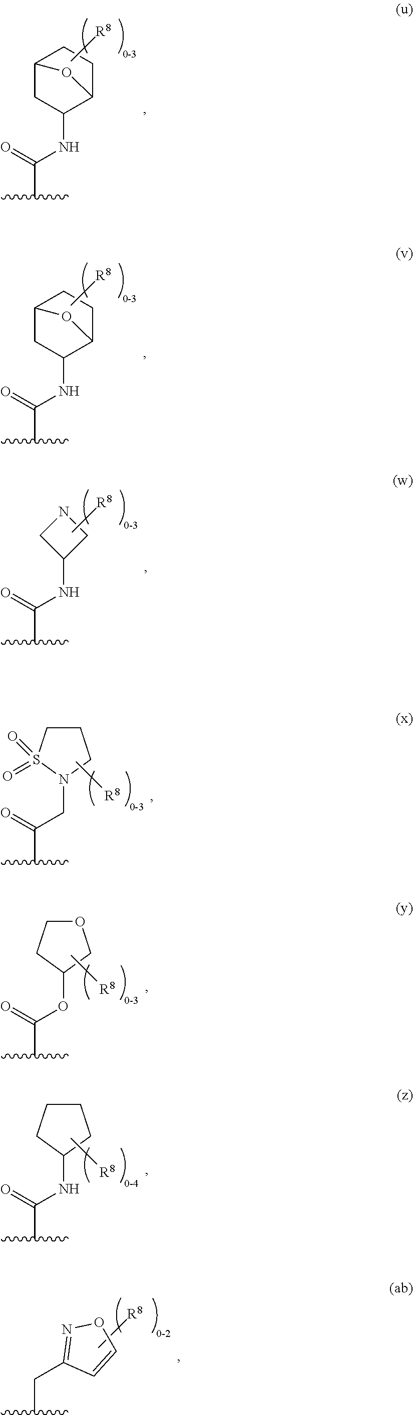

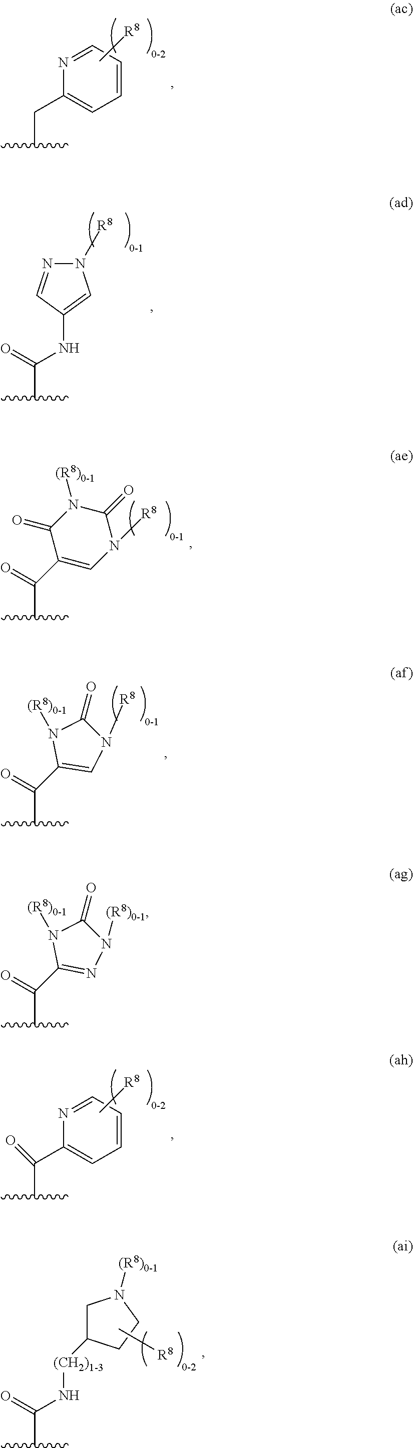

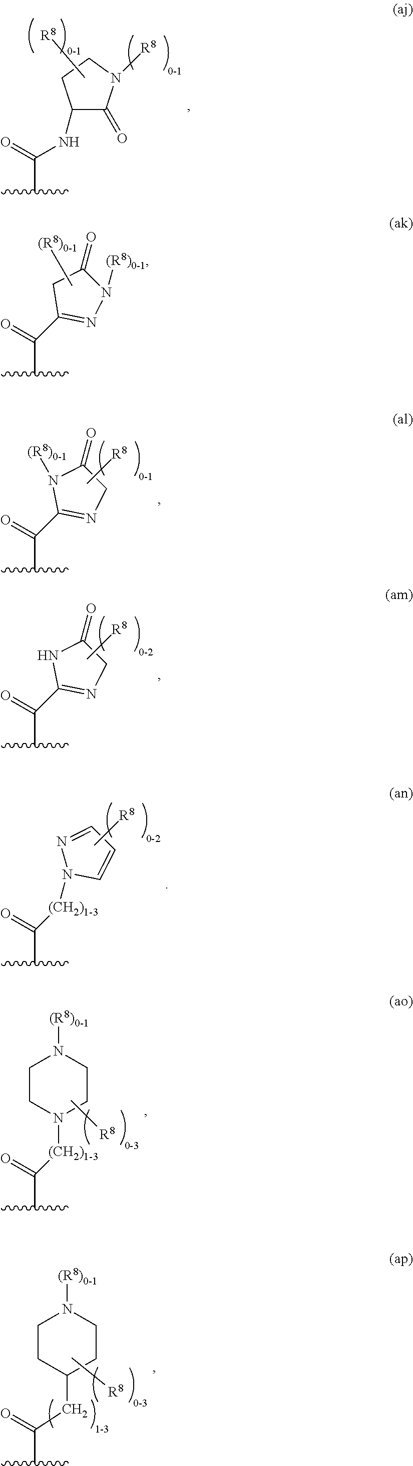

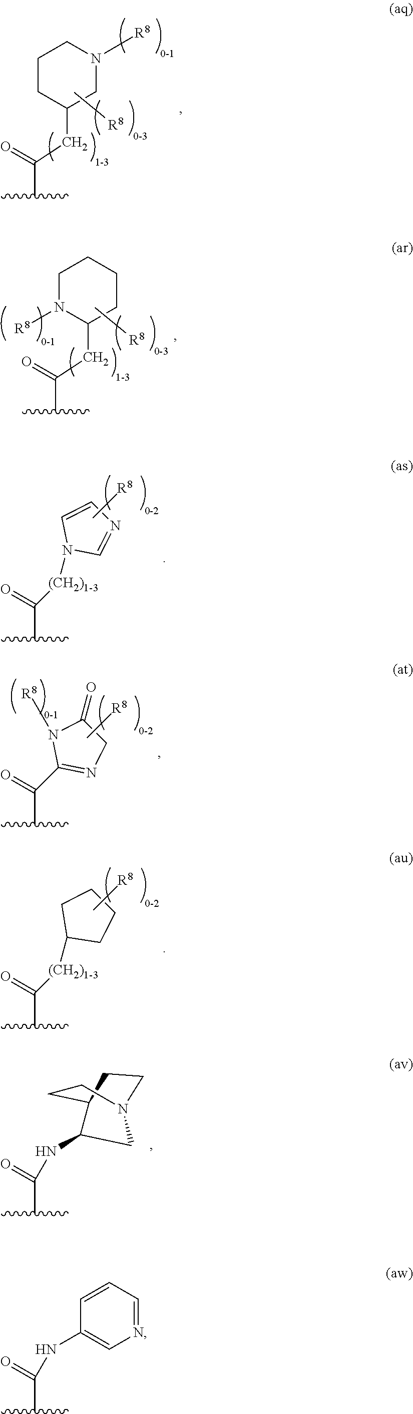

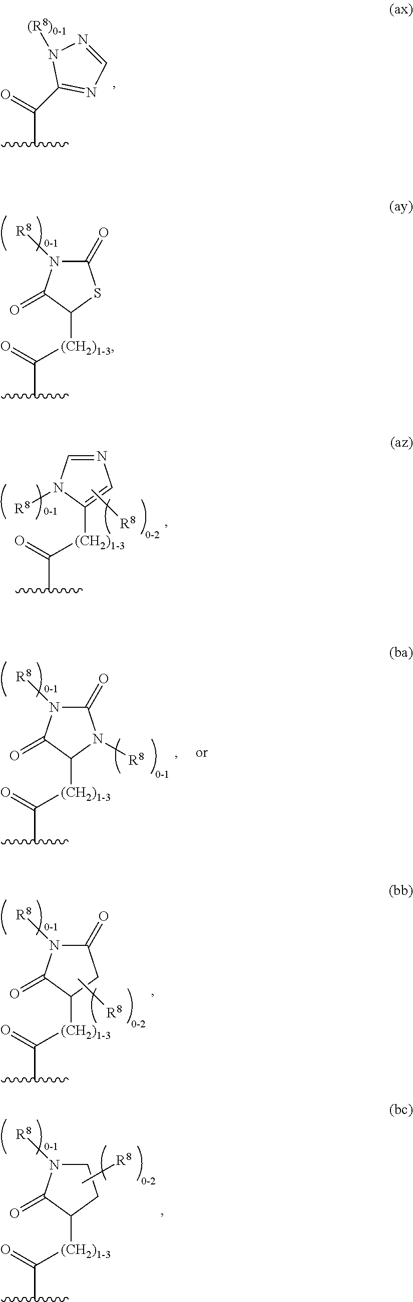

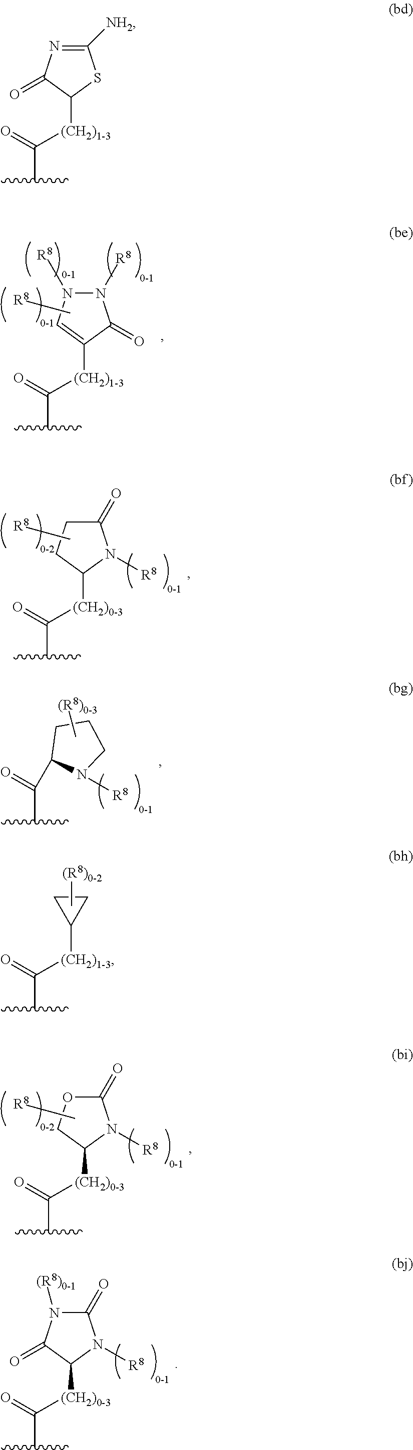

Embodiment 4(ii)(e) relates to Embodiment 4(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 4(iii) relates to any one of the Embodiments 4(ii), 4(ii)(a), 4(ii)(b), 4(ii)(c), 4(ii)(d), or 4(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 4(iv) relates to any one of the Embodiments 4(ii), 4(ii)(a), 4(ii)(b), 4(ii)(c), 4(ii)(d), or 4(ii)(e) wherein R² is saturated 7-9 membered heterocycloalkyl comprising at least one nitrogen.

Embodiment 4(v) relates to any one of the Embodiments 4(ii), 4(ii)(a), 4(ii)(b), 4(ii)(c), 4(ii)(d), or 4(ii)(e) wherein R² is

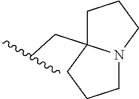 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 4(vi) relates to Embodiment 4(ii) having the Formula of (HD):

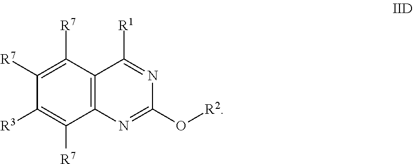

Embodiment 4(vi)(a) relates to Embodiment 4(vi) wherein G is H.
Embodiment 4(vi)(b) relates to Embodiment 4(vi) wherein G is not H.

Embodiment 4(vii) relates to any one of the Embodiments 4(vi), 4(vi)(a), or 4(vi)(b) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 4(viii) relates to any one of the Embodiments 4(vi), 4(vi)(a), or 4(vi)(b) wherein R² is saturated 7-9 membered heterocycloalkyl comprising at least one nitrogen.

Embodiment 4(ix) relates to any one of the Embodiments 4(vi), 4(vi)(a), or 4(vi)(b) wherein R² is

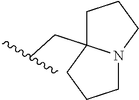 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 4(x) relates to Embodiment 4(ii) wherein when G is H, then R² is

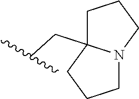 [SMILES: *CC12CCCN1CCC2]

that is substituted with 0-3 R⁴.

Embodiment 5(ii) of this disclosure relates to the compound according to the compound of any one of Embodiments 1(ii), 2(ii), 3(ii), or 4(ii) having one of the following formulae:

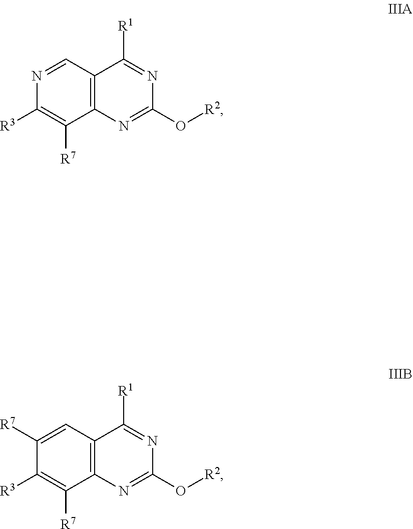

or a pharmaceutically acceptable salt, a solvate, a tautomer, a stereoisomer, or a deuterated analog thereof, wherein each R⁷ is independently H, F, Cl, C₁-C₃alkyl, or C₁-C₃haloalkyl.
Embodiment 5(ii)(a) relates to Embodiment 5(ii) wherein G is H, provided that when the compound has the Formula (IIIA), R⁷ is not H.
Embodiment 5(ii)(b) relates to Embodiment 5(ii) wherein G is not H.
Embodiment 5(ii)(c) relates to Embodiment 5(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 5(ii)(d) relates to Embodiment 5(ii) wherein G has one of the following Formulae:

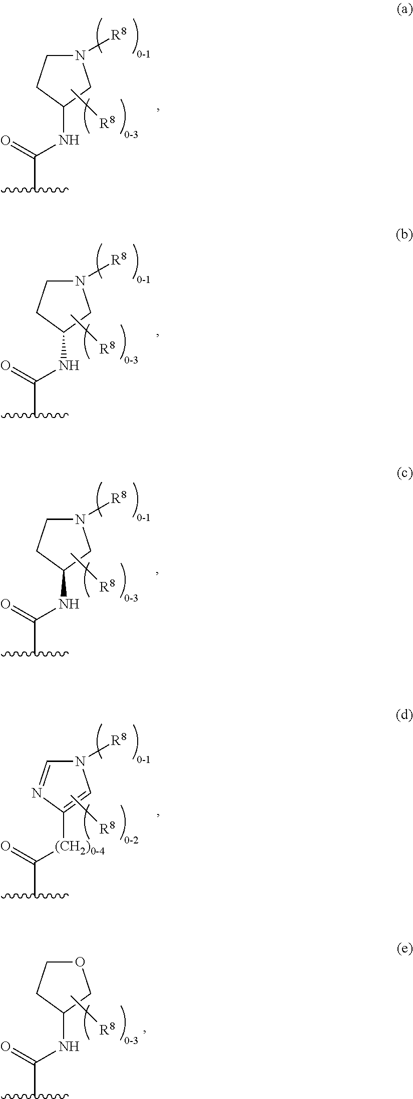

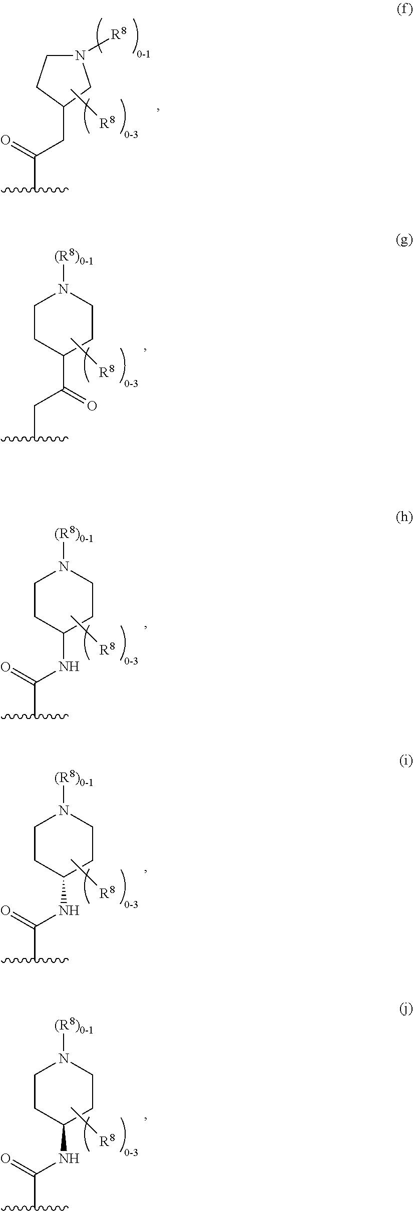

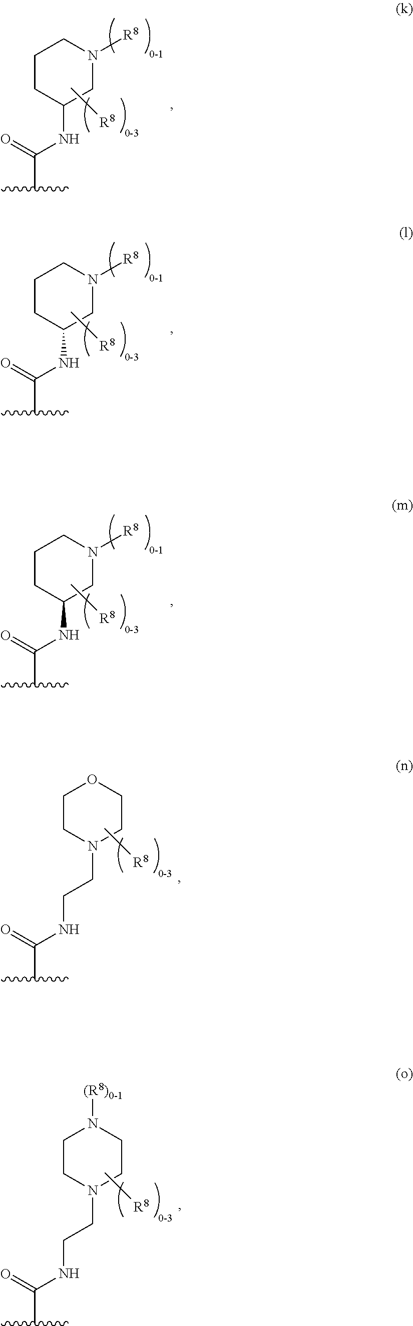

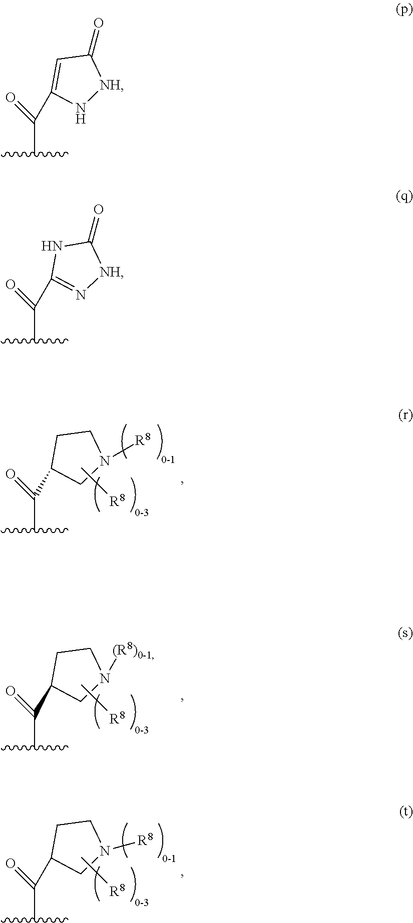

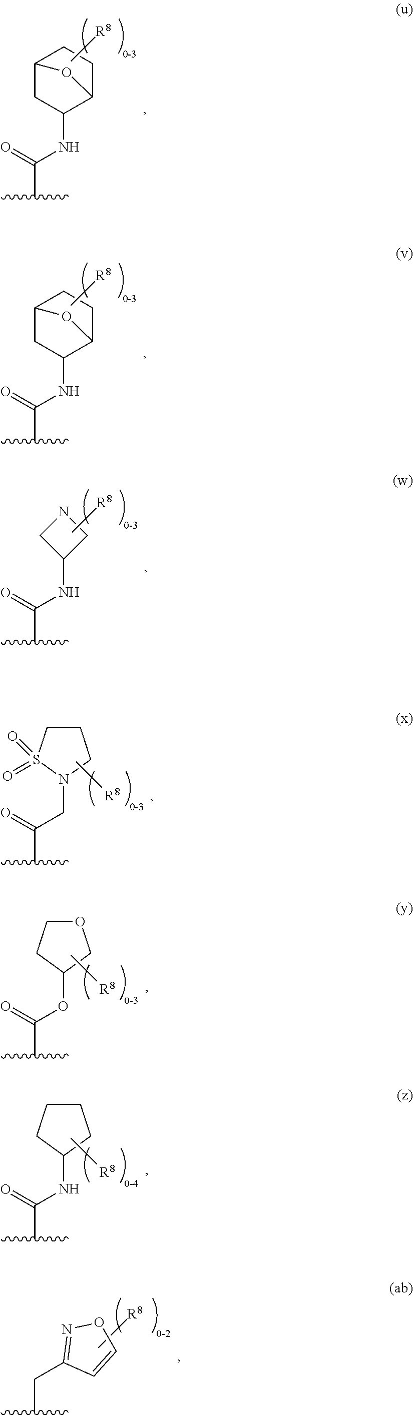

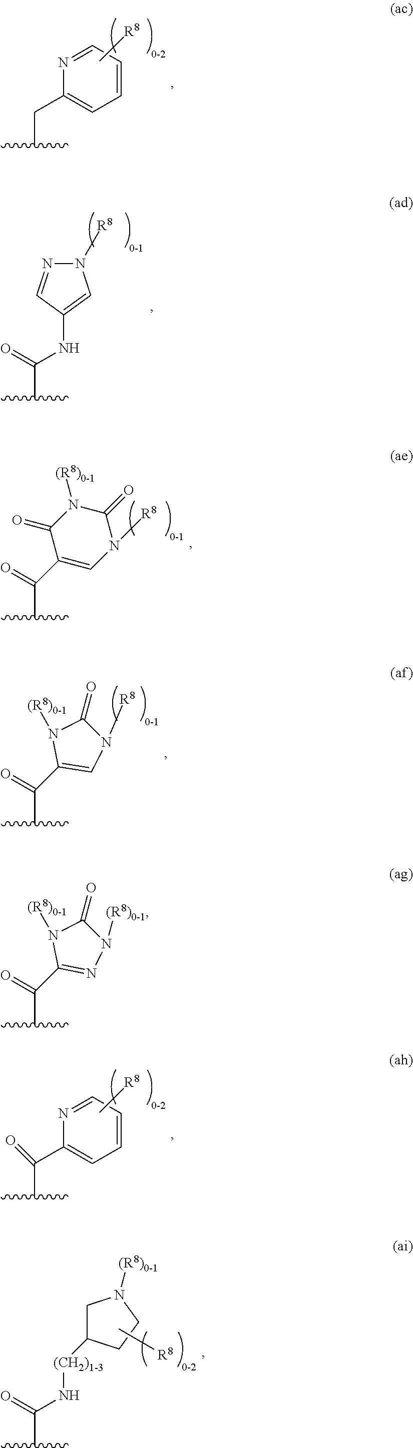

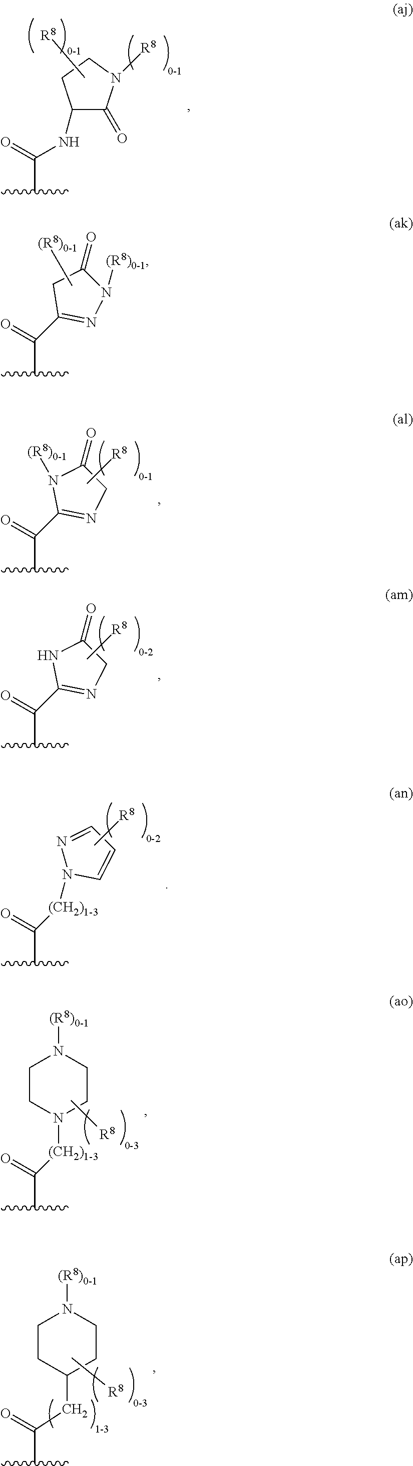

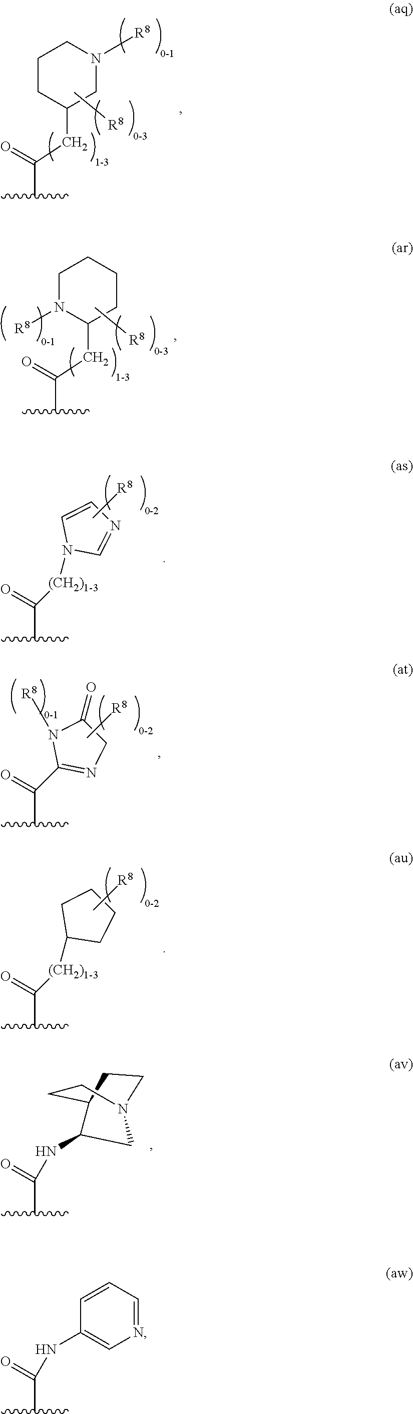

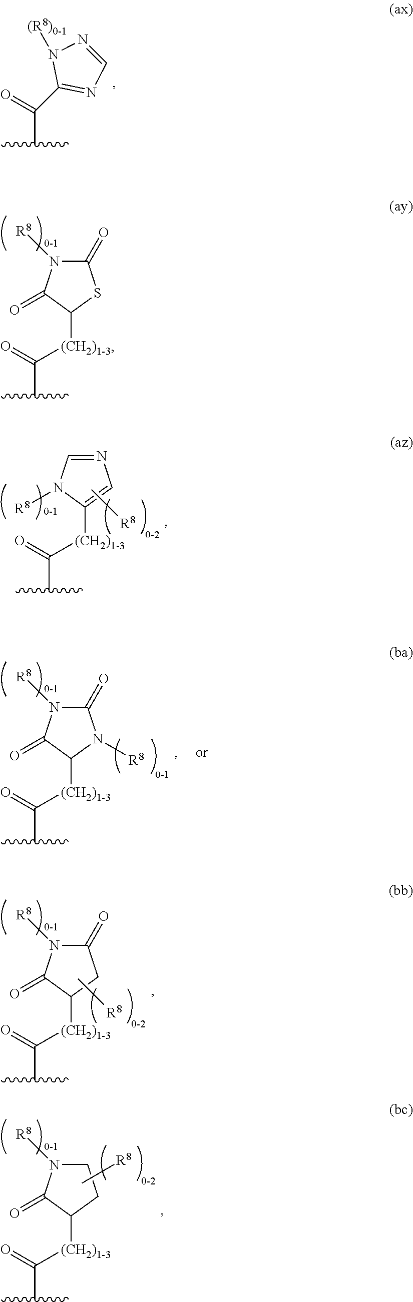

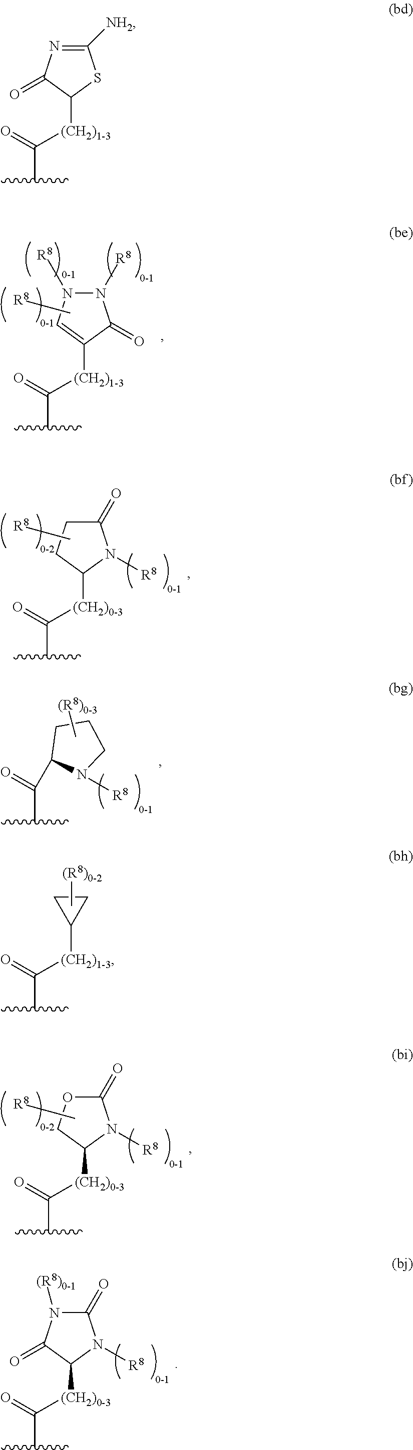

Embodiment 5(ii)(e) relates to Embodiment 5(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 5(iii) relates to any one of the Embodiments 5(ii), 5(ii)(a), 5(ii)(b), 5(ii)(c), 5(ii)(d), or 5(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 5(iv) relates to any one of the Embodiments 5(ii), 5(ii)(a), 5(ii)(b), 5(ii)(c), 5(ii)(d), or 5(ii)(e) wherein R² is saturated 7-9 membered heterocycloalkyl comprising at least one nitrogen.

Embodiment 5(v) relates to any one of the Embodiments 5(ii), 5(ii)(a), 5(ii)(b), 5(ii)(c), 5(ii)(d), or 5(ii)(e) wherein R² is

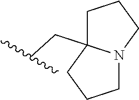 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 5(vi) relates to Embodiment 5(ii) having the Formula of (IIIB):

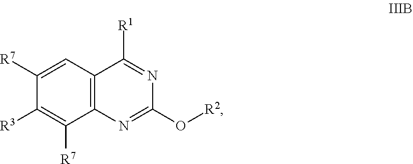

Embodiment 5(vi)(a) relates to Embodiment 5(vi) wherein G is H.
Embodiment 5(vi)(b) relates to Embodiment 5(vi) wherein G is not H.

Embodiment 5(vii) relates to any one of the Embodiments 5(vi), Embodiments 5(vi)(a), or 5(vi)(b) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 5(viii) relates to any one of the Embodiments 5(vi), Embodiments 5(vi)(a), or 5(vi)(b) wherein R² is saturated 7-9 membered heterocycloalkyl comprising at least one nitrogen.

Embodiment 5(ix) relates to any one of the Embodiments 5(vi), Embodiments 5(vi)(a), or 5(vi)(b) wherein R² is

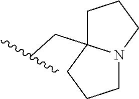 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 5(x) relates to Embodiment 5(ii) wherein when G is H, then R² is

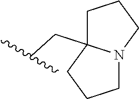 [SMILES: *CC12CCCN1CCC2]

that is substituted with 0-3 R⁴.

Embodiment 6(ii) of this disclosure relates to the compound according to any one of Embodiments 1(ii), 2(ii), 3(ii), 4(ii), or 5(ii) having one of the following formulae:

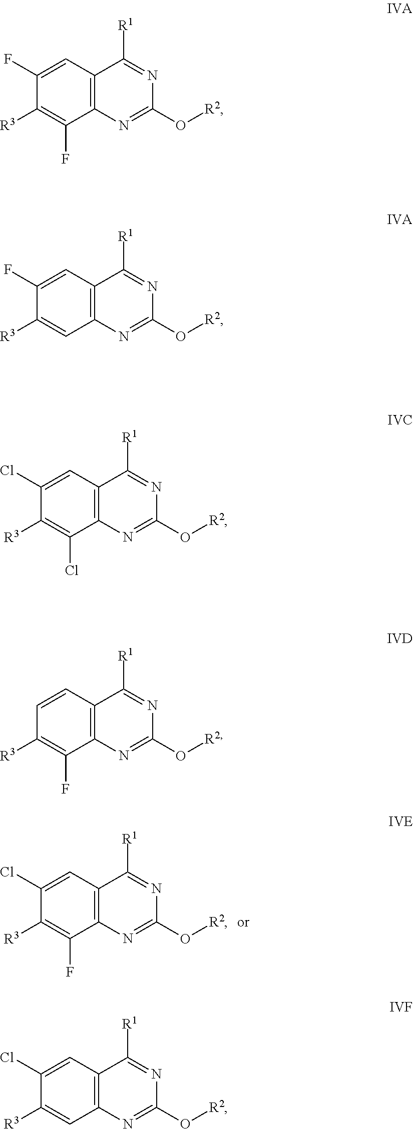

or a pharmaceutically acceptable salt, a solvate, a tautomer, a stereoisomer, or a deuterated analog thereof.
Embodiment 6(ii)(a) relates to Embodiment 6(ii) wherein G is H.
Embodiment 6(ii)(b) relates to Embodiment 6(ii) wherein G is not H.
Embodiment 6(ii)(c) relates to Embodiment 6(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 6(ii)(d) relates to Embodiment 6(ii) wherein G has one of the following Formulae:

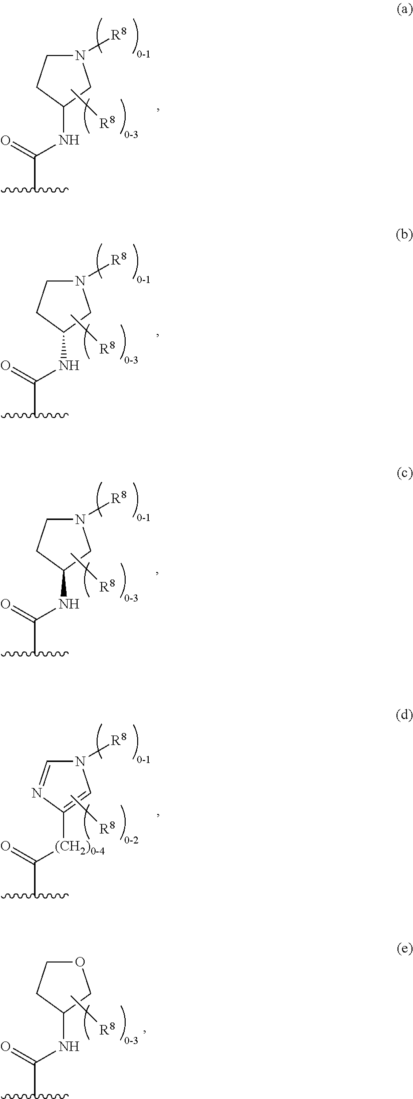

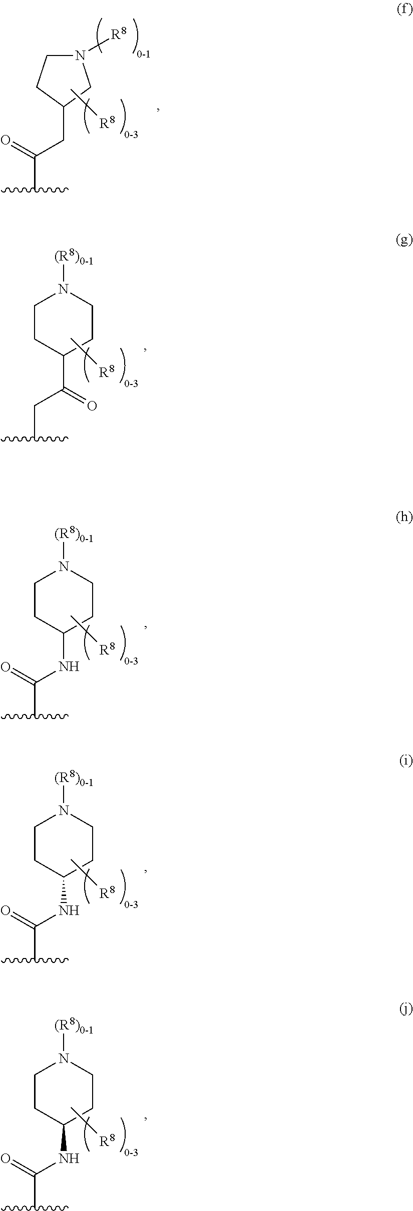

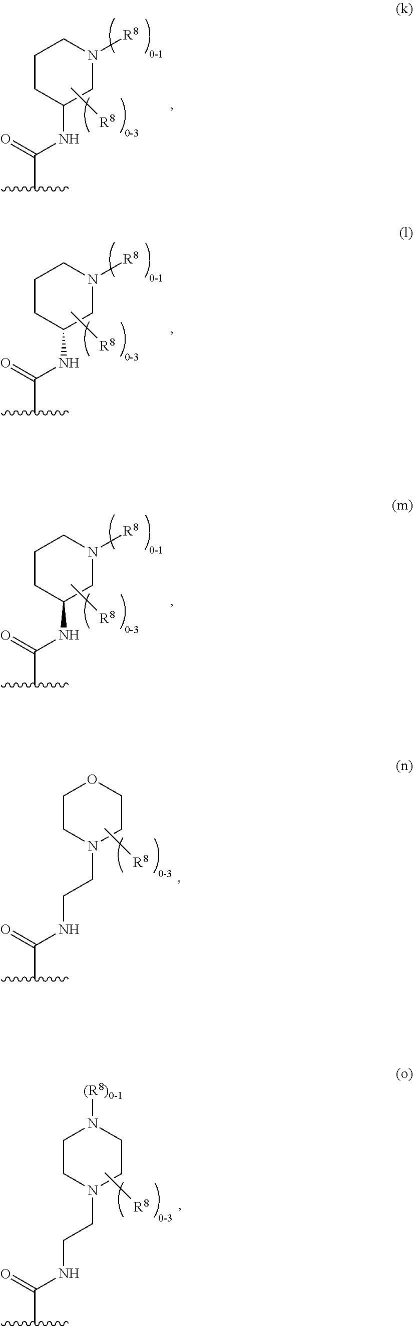

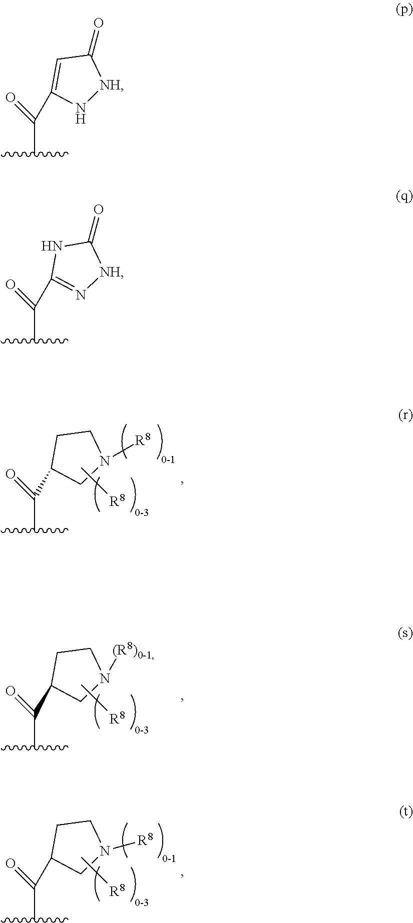

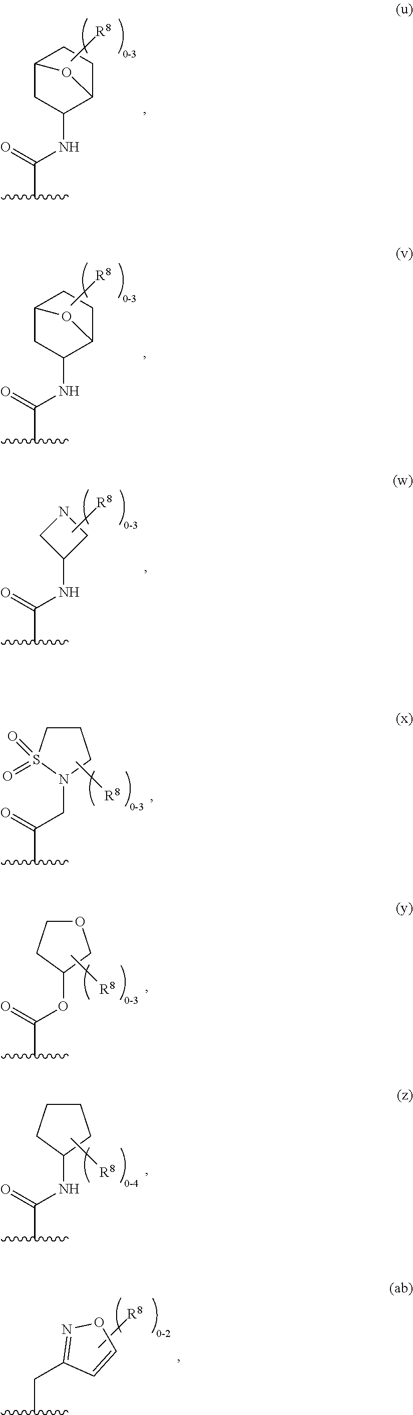

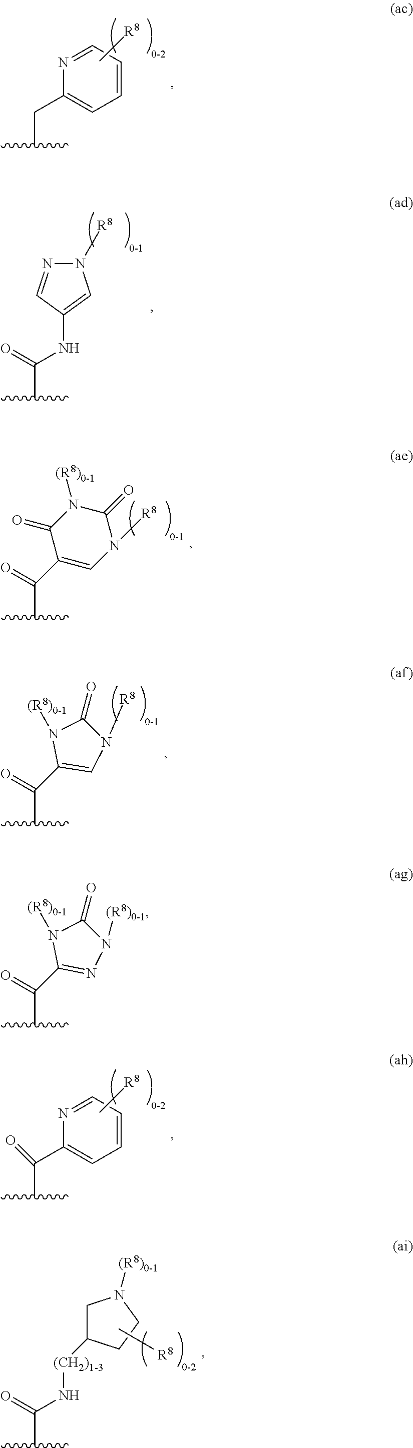

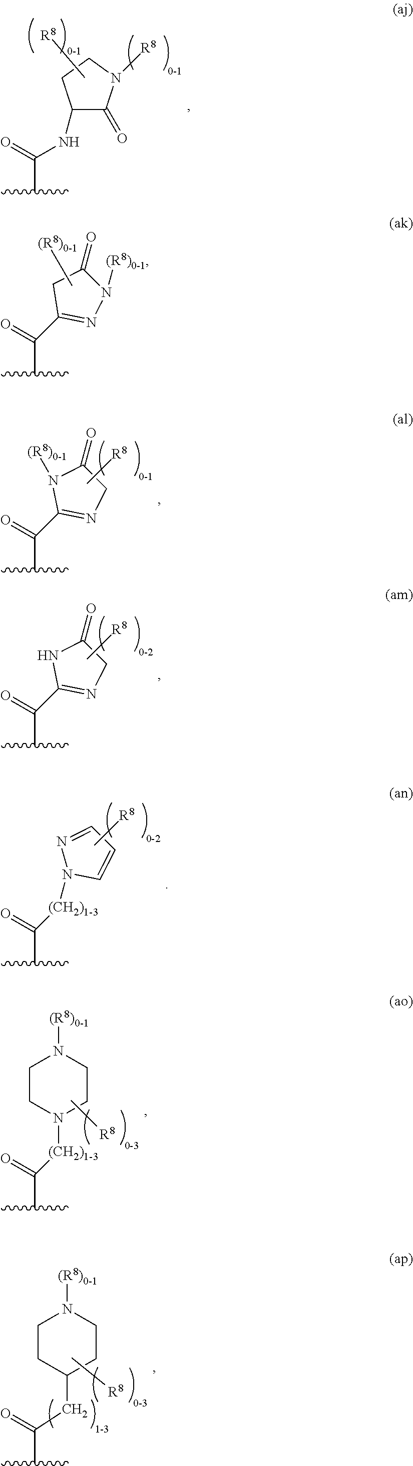

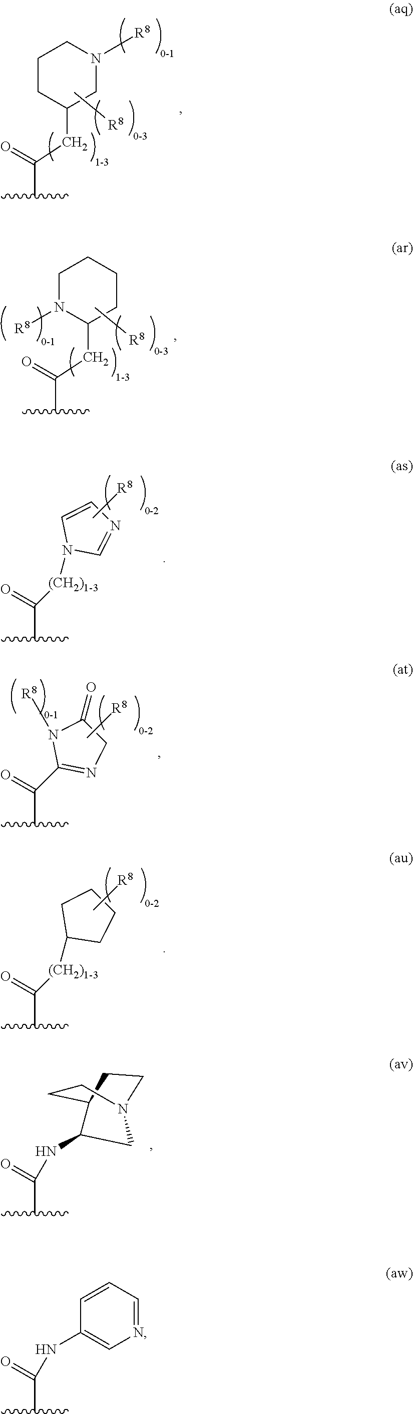

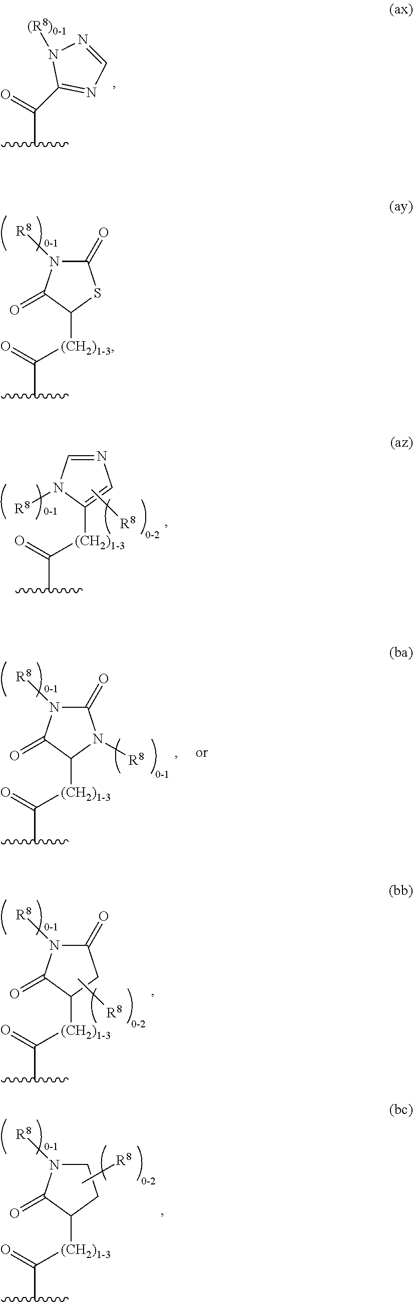

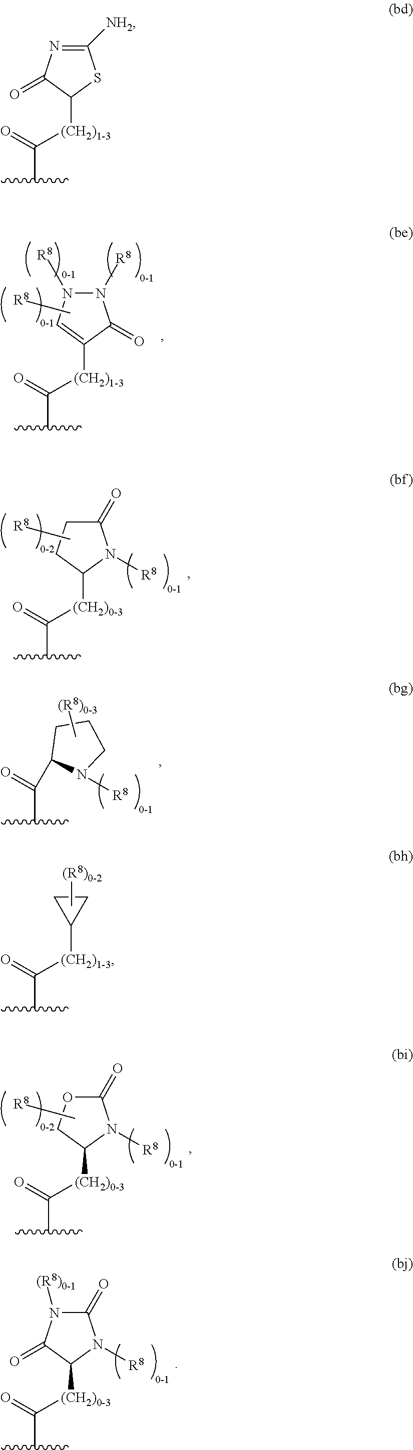

Embodiment 6(ii)(e) relates to Embodiment 6(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 6(iii) relates to any one of Embodiments 6(ii), 6(ii)(a), 6(ii)(b), 6(ii)(c), 6(ii)(d), or 6(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 6(iv) relates to any one of Embodiments 6(ii), 6(ii)(a), 6(ii)(b), 6(ii)(c), 6(ii)(d), or 6(ii)(e) wherein R² is saturated 7-9 membered heterocycloalkyl comprising at least one nitrogen.

Embodiment 6(v) relates to any one of Embodiments 6(ii), 6(ii)(a), 6(ii)(b), 6(ii)(c), 6(ii)(d), or 6(ii)(e) wherein R² is

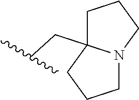 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 6(vi) relates to Embodiment 6(ii) wherein when G is H, then R² is

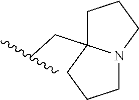 [SMILES: *CC12CCCN1CCC2]

that is substituted with 0-3 R⁴.

Embodiment 7(ii) of this disclosure relates to the compound according to any one of Embodiments 1(ii), 2(ii), 3(ii), 4(ii), 5(ii), or 6(ii) wherein:
R¹ is a 7-9 membered bridged heterocycloalkyl having at least one nitrogen atom, wherein R¹ is substituted with 0-3 T¹ and 1 G;
R² is —C₀-C₅alkyl-4-10 membered heterocycloalkyl substituted with 0-4 R⁴ and 0-1 J; —C₀-C₅alkyl-5-10 membered heteroaryl substituted with 0-3 R⁴ and 0-1 J; or C₁-C₆alkyl substituted with 1-2 J groups;
R³ is phenyl, naphthyl, 5-10 membered heteroaryl, or 5-10 membered heterocycloalkyl, wherein the naphthyl, 5-10 membered heteroaryl, and 5-10 membered heterocycloalkyl are each substituted with 0-4 R⁵, and wherein the phenyl is substituted with 0-4 R⁵ and 0-1M;
each R⁴ is independently C₁-C₅alkyl, halogen, hydroxyl, CN, C₁-C₅haloalkyl, C₁-C₅hydroxyalkyl, C₁-C₅cyanoalkyl, or C₁-C₅alkoxy, provided that when R⁴ is attached to a nitrogen atom, R⁴ is not halogen, hydroxyl, or CN;
each R⁵ is independently C₁-C₅alkyl, C₁-C₅alkynyl, C₁-C₅alkoxy, halogen, hydroxyl, CN, C₁-C₅haloalkyl, C₁-C₅cynoalkyl, or C₁-C₅hydroxyalkyl
each R⁸ is independently C₁-C₅alkyl, halogen, hydroxyl, —NH₂, —N(H)C₁-C₅alkyl, —N(C₁-C₅alkyl)₂, CN, C₁-C₅haloalkyl, or C₁-C₅hydroxyalkyl provided that when R⁸ is attached to nitrogen, R⁸ is not halogen, hydroxyl, —NH₂, —N(H)C₁-C₅alkyl, —N(C₁-C₅alkyl)₂, or CN;
each R⁹ is independently halogen, hydroxyl, CN, C₁-C₅haloalkyl, or C₁-C₅hydroxyalkyl;
each R¹⁰ is independently H, C₁-C₅alkyl, or C₁-C₅haloalkyl;
each T¹ is independently C₁-C₅alkyl, halogen, hydroxyl, C₁-C₅haloalkyl, or C₁-C₅hydroxyalkyl, provided that when T¹ is attached to nitrogen, T¹ is not halogen or hydroxyl;
T² is H, CN, —C(O)OR¹⁰, C₁-C₅alkoxy, N(R¹⁰)₂, —C(O)N(R¹⁰)₂, —SO₂N(R¹⁰)₂, —SO₂R¹⁰, —N(R¹⁰)C(O)—C₁-C₅alkyl, —O—C(O)—C₁—CN alkyl. C₃-C₆cycloalkyl, 4-10 membered heterocycloalkyl, 7-9 membered bridged heterocycloalkyl, phenyl, naphthyl, or 5-10 membered heteroaryl, wherein T² substituted with 0-4 R⁸,
provided that when L¹ and L² are both absent, T² is not CN, C₁-C₅alkoxy, N(R¹⁰)₂, —N(R¹⁰)C(O)—C₁-C₅alkyl, or —O—C(O)—C₁-C₅alkyl, and provided that when L¹ and L² taken together are —C(O)—, —C(O)N(H)—, —C₁-C₅alkyelene-C(O)N(H)—, —C₁-C₅alkylene-C(O)—. —SO₂—, or —SO₂NH, T² is not CN, —C(O)OR¹⁰, C₁-C₅alkoxy, N(R¹⁰)₂, —C(O)N(R¹⁰)₂, —SO₂N(R¹⁰)₂, —N(R¹⁰)C(O)—C₁-C₅alkyl, or —O—C(O)—C₁-C₅alkyl;
L¹ is absent, —C(O)—, —C(O)N(H)—, —C₁-C₅alkyelene-C(O)N(H)—, —C₁-C₅ alkelene-C(O)—, —SO₂—, or —SO₂N(H)—; and
L² is absent, or C₁-C₅alkylene substituted with 0-4 R⁹, provided that when L¹ is —C(O)— and L² is absent, T² is not H; and
M is —C(O)NH₂, —NH₂C(O)CH₃, —N(H)SO₂CH₃, or —NH₂, provided that M is not attached to a nitrogen atom.
Embodiment 7(ii)(a) relates to Embodiment 7(ii) wherein G is H.
Embodiment 7(ii)(b) relates to Embodiment 7(ii) wherein G is not H.
Embodiment 7(ii)(c) relates to Embodiment 7(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 7(ii)(d) relates to Embodiment 7(ii) wherein G has one of the following Formulae:

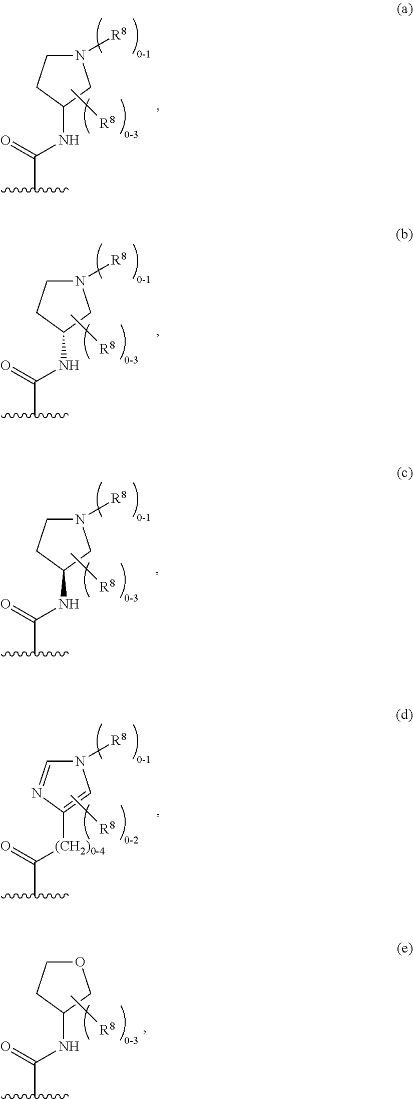

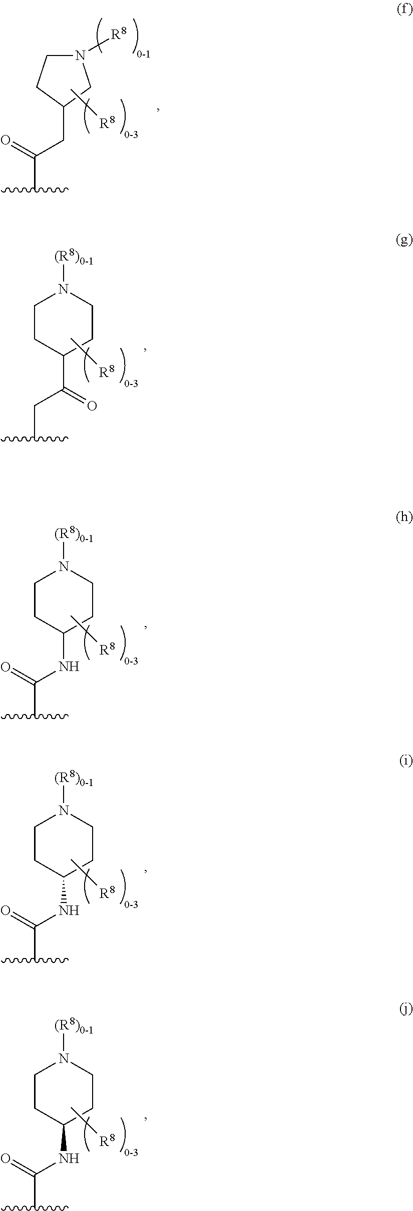

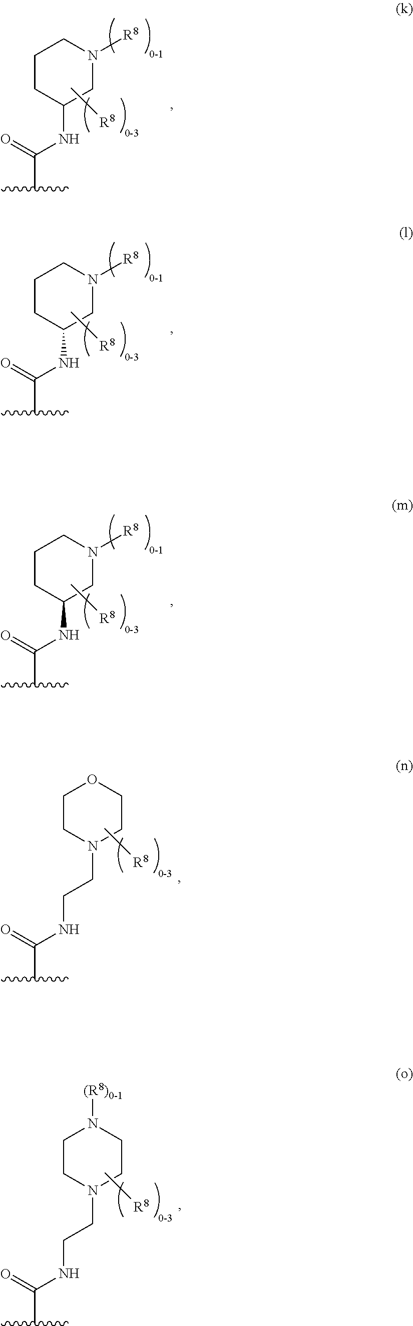

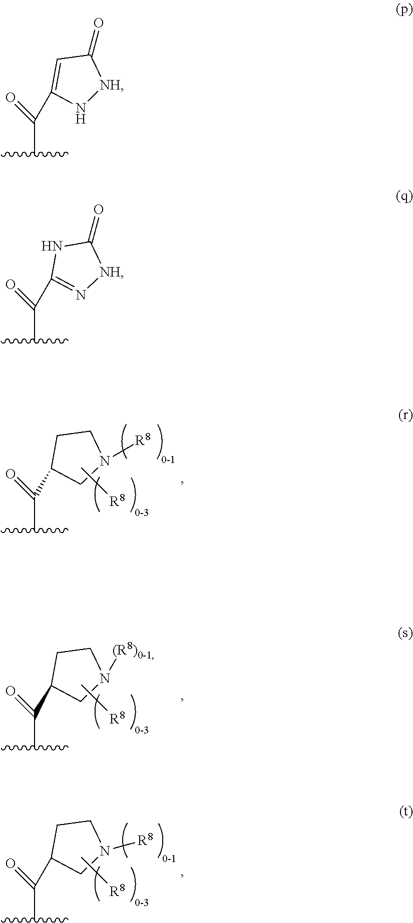

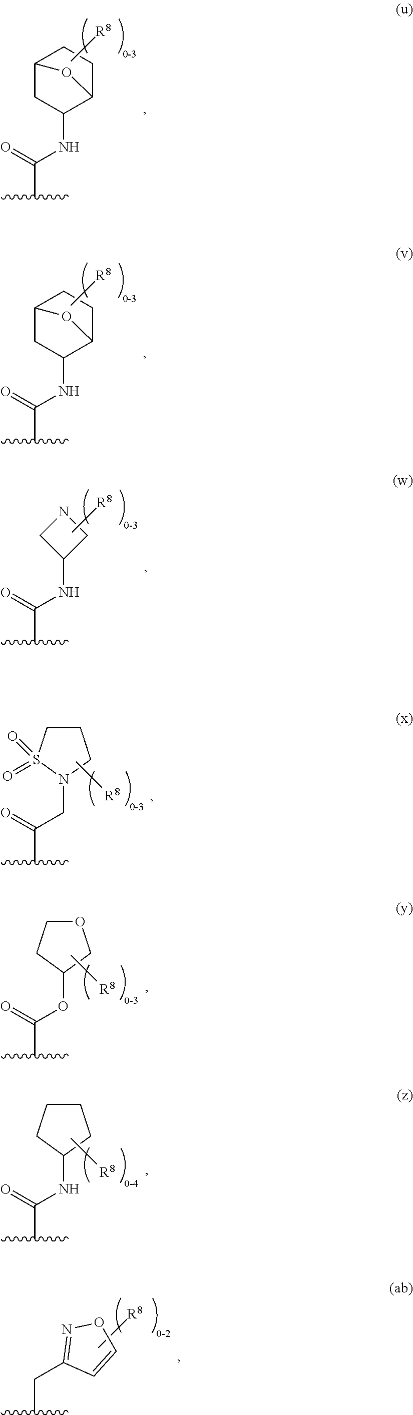

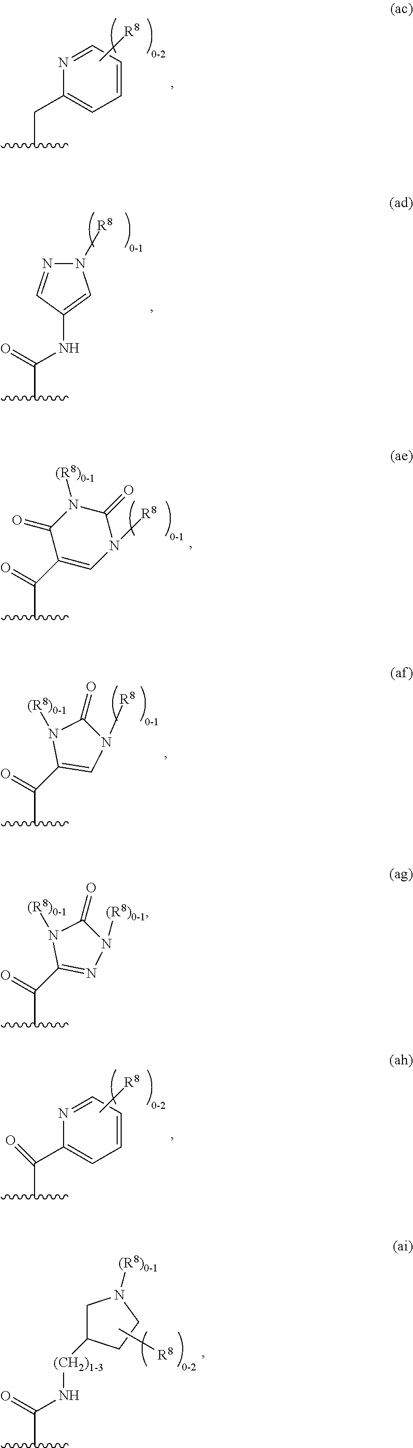

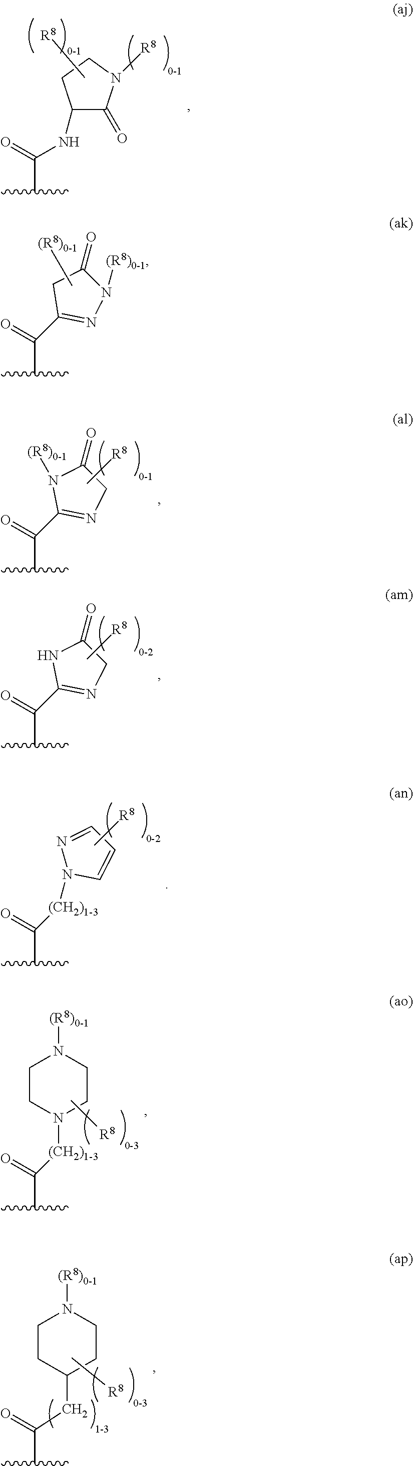

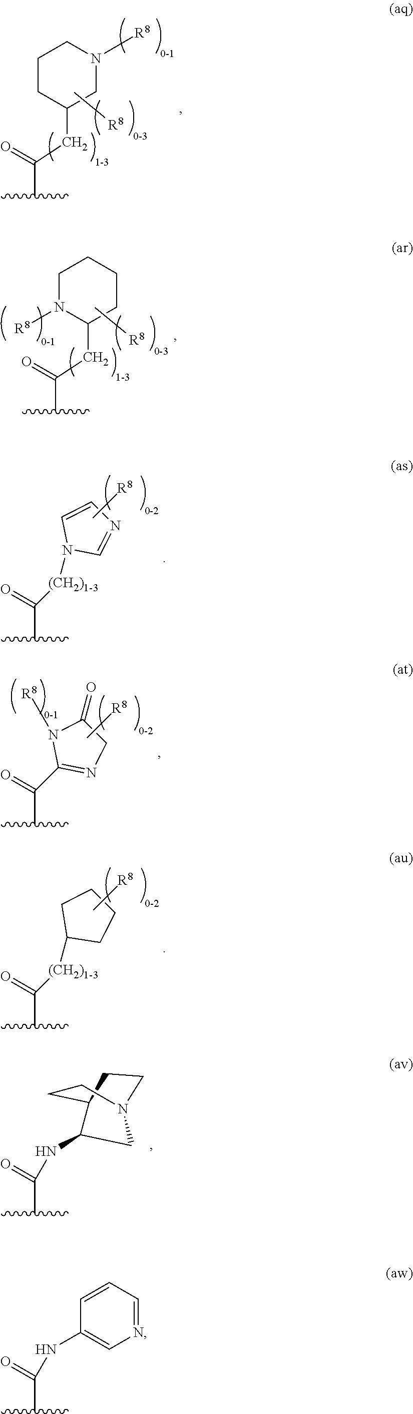

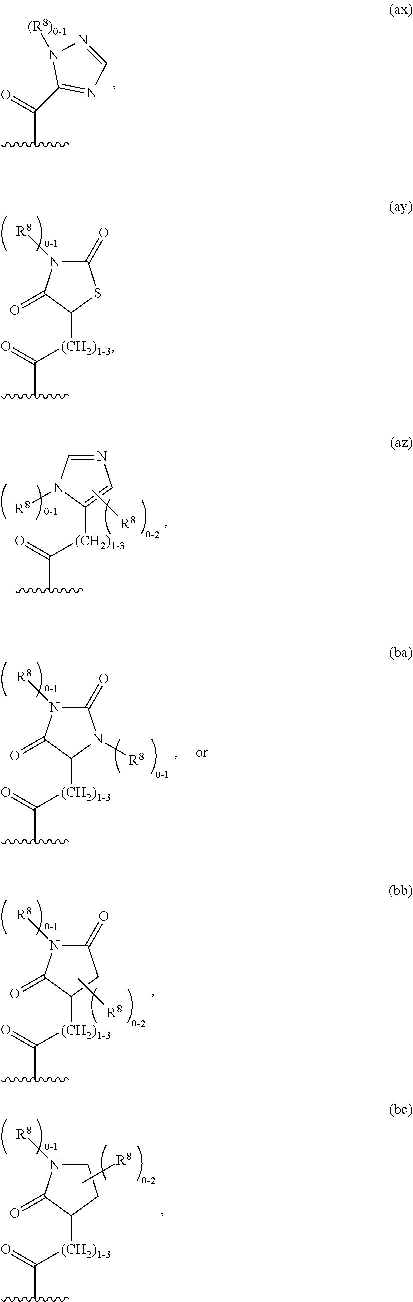

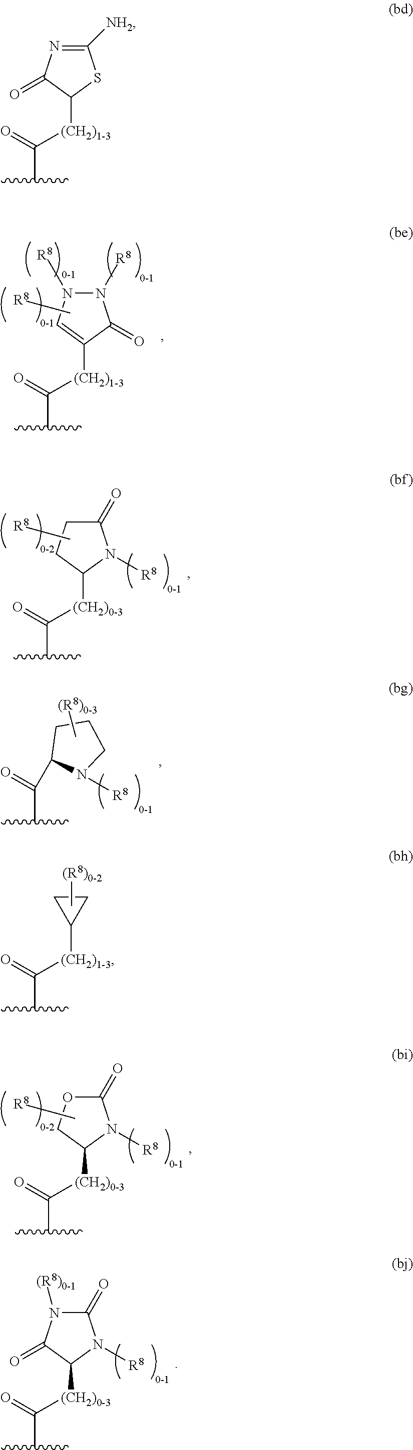

Embodiment 7(ii)(e) relates to Embodiment 7(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 7(iii) relates to any one of Embodiments 7(ii), 7(ii)(a), 7(ii)(b), 7(ii)(c), 7(ii)(d), or 7(ii)(e) wherein R² is saturated 7-9 membered heterocycloalkyl comprising at least one nitrogen.

Embodiment 7(iv) relates to any one of Embodiments 7(ii), 7(ii)(a), 7(ii)(b), 7(ii)(c), 7(ii)(d), or 7(ii)(e) wherein R² is

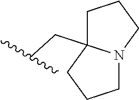 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 7(v) relates to Embodiment 7(ii) wherein when G is H, then R² is

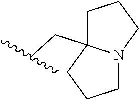 [SMILES: *CC12CCCN1CCC2]

that is substituted with 0-3 R⁴.

Embodiment 8(ii) of this disclosure relates to the compound according to any of Embodiments 1(ii), 2(ii), 3(ii), 4(ii), 5(ii), 6(ii), or 7(ii), wherein:
R¹ is a 7-9 membered bridged heterocycloalkyl having at least one nitrogen atom, wherein R¹ is substituted with 0-2 T¹ and 1 G;
R² is 5-10 membered heterocyloalkyl-C₁-C₄alkyl substituted with 0-3 R⁴ or C₁-C₄alky 1 substituted with 1-2 J groups;
R³ is phenyl, naphthyl, 5-10 membered heteroaryl, or a 5-10 membered heterocycloalkyl, wherein the naphthyl, 5-10 membered heteroaryl, and 5-10 membered heterocycloalkyl are each substituted with 0-3 R⁵, and wherein the phenyl is substituted with 0-4 R⁵ and 0-1M;
each R⁴ is independently C₁-C₄alkyl, halogen, hydroxyl, CN, C₁-C₄haloalkyl, C₁-C₄hydroxyalkyl, or C₁-C₄alkoxy, provided that when R⁴ is attached to a nitrogen atom, R⁴ is not halogen, hydroxyl, or CN;
each R⁵ is independently C₁-C₄alkyl, C₁-C₄alkynyl, halogen, hydroxyl, CN, C₁-C₄haloalkyl, C₁-C₄cynoalkyl, or C₁-C₄hydroxyalkyl;
each R⁸ is independently C₁-C₄alkyl, halogen, hydroxyl, —NH₂, —N(H)C₁-C₄alkyl, —N(C₁-C₄alkyl)₂, CN, C₁-C₄haloalkyl, or C₁-C₄hydroxyalkyl, provided that when R⁸ is attached to nitrogen, R⁸ is not halogen, hydroxyl, —NH₂, —N(H)C₁-C₅alkyl. —N(C₁-C₅alkyl)₂, or CN;
each R⁹ is independently halogen, hydroxyl, CN, C₁-C₄haloalkyl, or C₁-C₄hydroxyalkyl;
each R¹⁰ is independently H, C₁-C₄alkyl, or C₁-C₄haloalkyl;
each T¹ is independently C₁-Chalky 1 halogen hydroxyl, C₁-C₄haloalkyl, or C₁-C₄hydroxyalkyl, provided that when T¹ is attached to nitrogen, T¹ is not halogen or hydroxyl;
T² is H, CN, —C(O)OR¹⁰, C₁-C₄alkoxy. —N(R¹⁰)₂, —C(O)N(R¹⁰)₂, SO₂N(R¹⁰)₂, —SO₂R¹⁰, —N(R¹⁰)C(O)—C₁-C₄alkyl. —O—C(O)—C₁-C₆alkyl, C₃-C₆cycloalkyl, 4-6 membered heterocycloalkyl, 7-8 membered bridged heterocycloalkyl, phenyl, naphthyl, or 5-6 membered heteroaryl, wherein T² is substituted with 0-4 R⁸,
provided that when L¹ and L² are both absent, T² is not CN, C₁-C₄alkoxy, N(R¹⁰)₂, —N(R¹⁰)C(O)—C₁-C₄alkyl, or —O—C(O)—C₁-C₄alkyl, and provided that when L¹ and L² taken together are —C(O)—, —C(O)N(H)—, —S(O)₂—, C₁-C₄alkyelene-C(O)N(H)—, or —C₁-C₄alkylene-C(O)—,
T² is not CN, —C(O)OR¹⁰, C₁-C₄alkoxy, —N(R¹⁰)₂, —C(O)N(R¹⁰)₂, SO₂N(R¹⁰)₂, —N(R¹⁰)C(O)—C₁-C₄alky 1, or —O—C(O)—C₁-C₄alkyl;
L¹ is absent, —C(O)—, —C(O)N(H)—, —S(O)₂—, —SO₂N(H)—, C₁-C₄alkyelene-C(O)N(H)—, —C₁-C₄alkylene-C(O)—, or —C₁-C₄alkylene-C(O)—; and
L² is absent, or C₁-C₄alkylene substituted with 0-2 R⁹, provided that when L¹ is —C(O)— and L² is absent, T² is not H.
Embodiment 8(ii)(a) relates to Embodiment 8(ii) wherein G is H.
Embodiment 8(ii)(b) relates to Embodiment 8(ii) wherein G is not H.
Embodiment 8(ii)(c) relates to Embodiment 8(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 8(ii)(d) relates to Embodiment 8(ii) wherein G has one of the following Formulae:

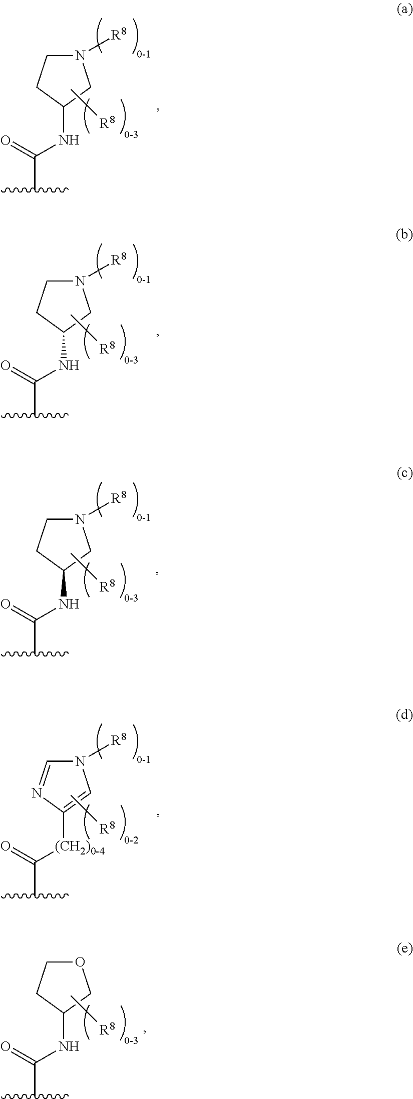

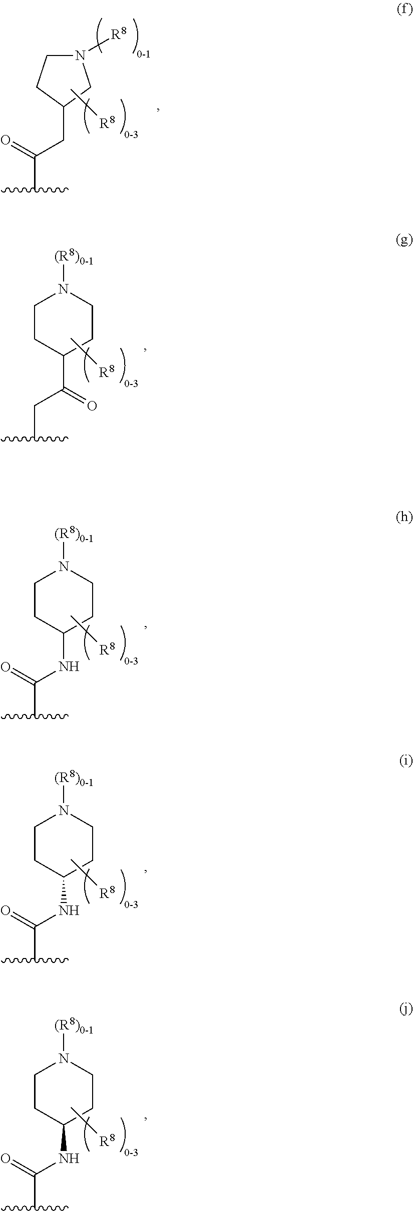

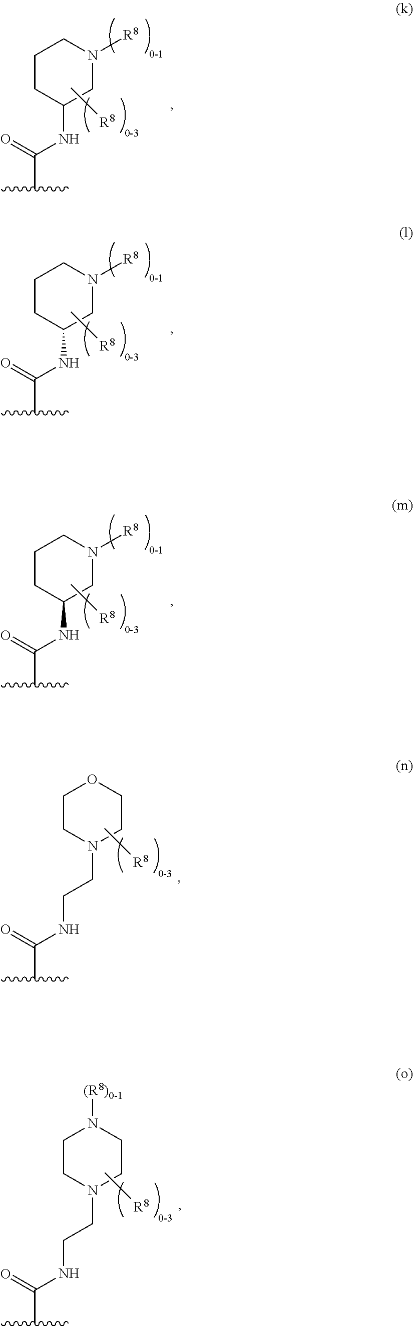

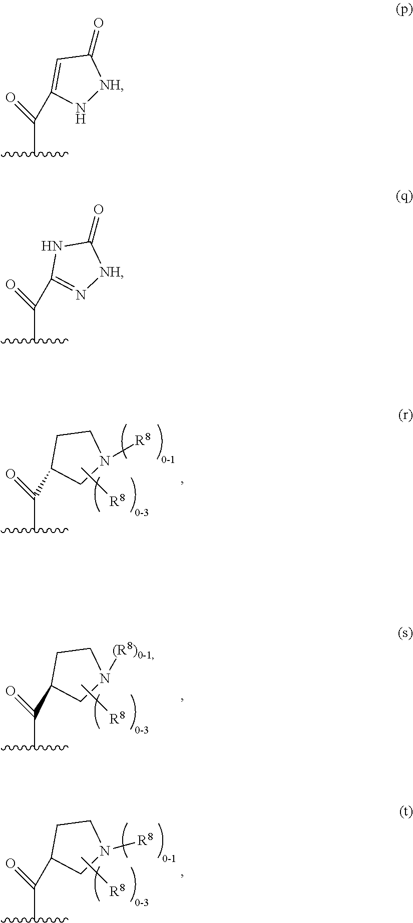

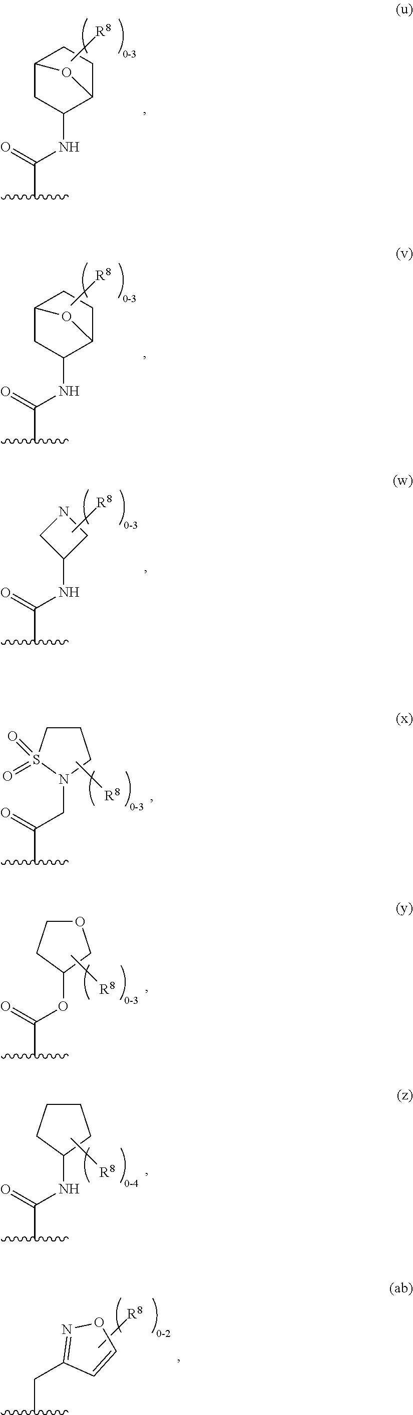

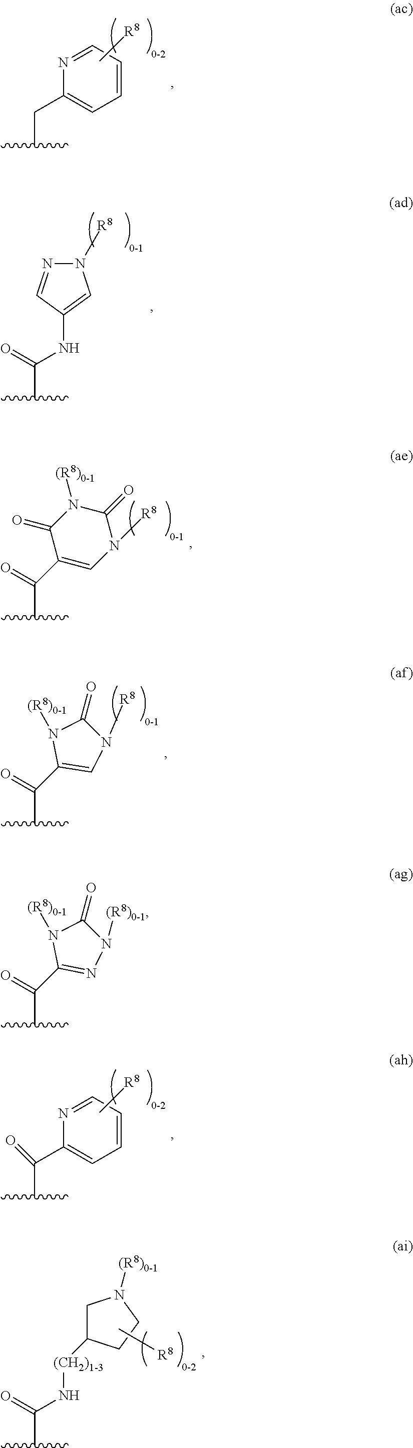

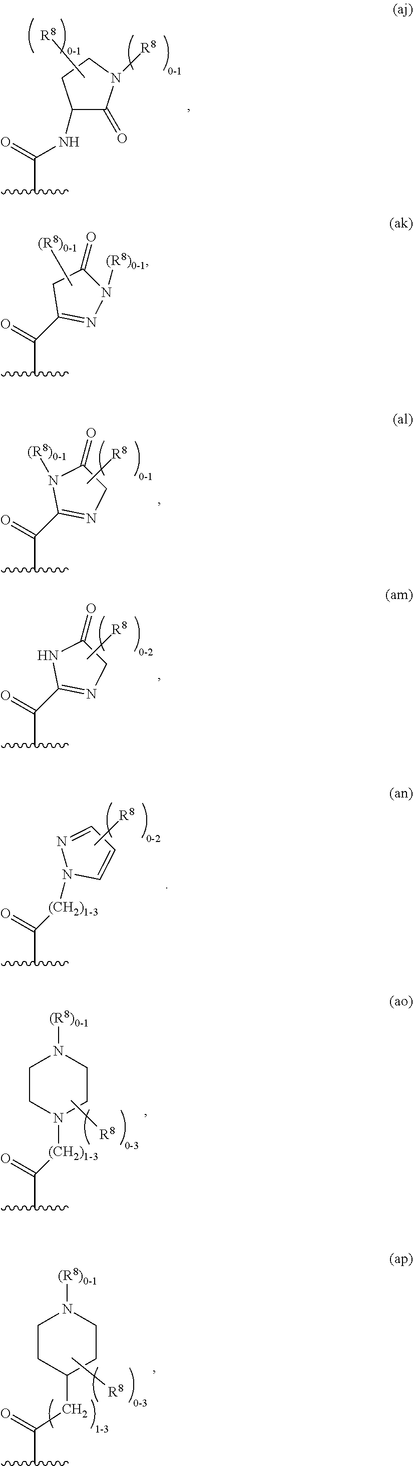

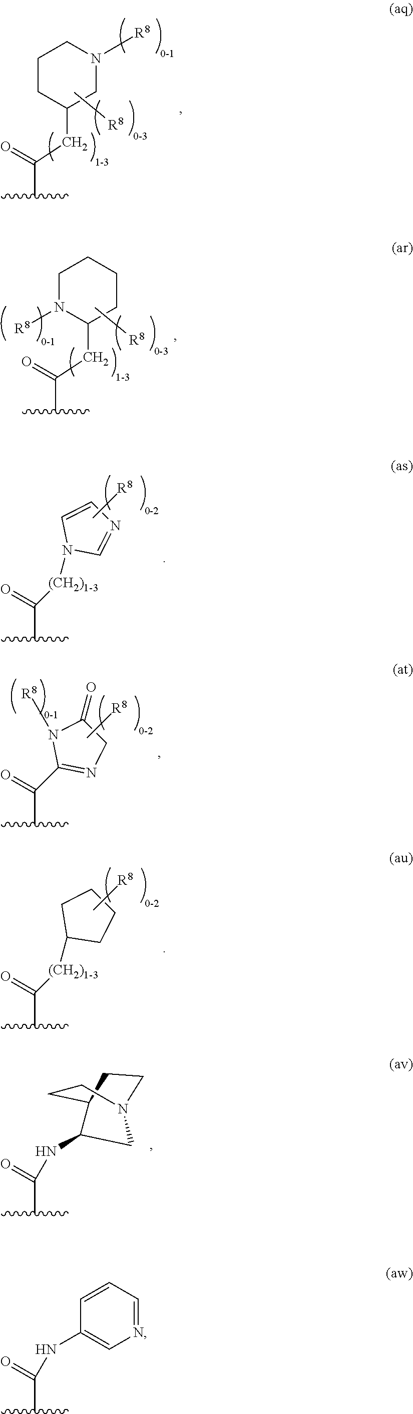

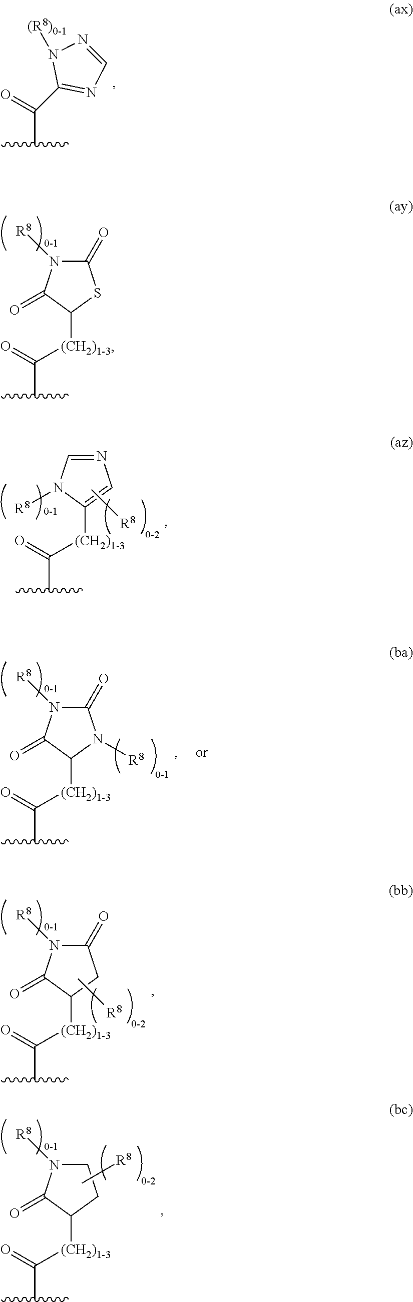

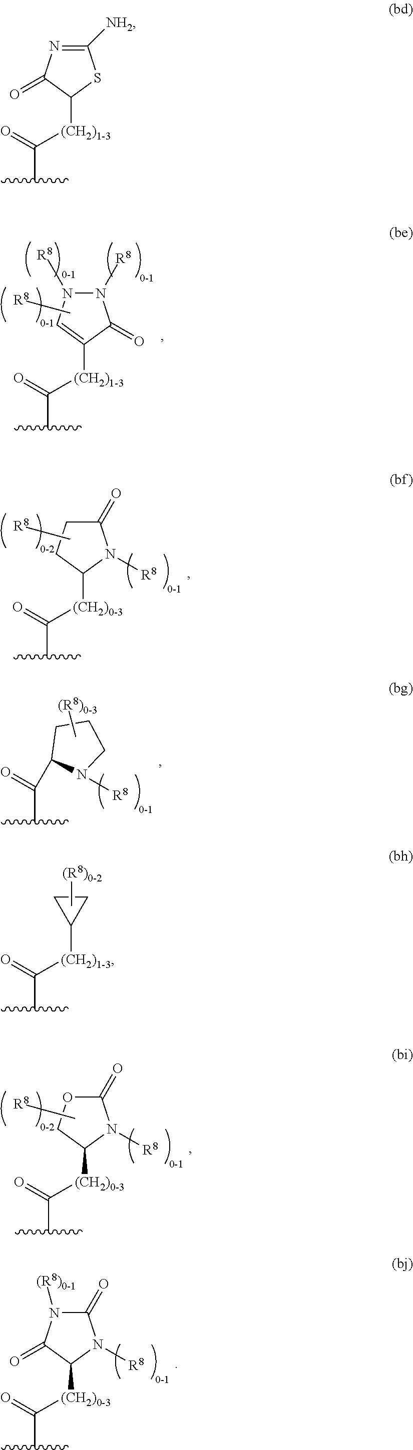

Embodiment 8(ii)(e) relates to Embodiment 8(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 8(iii) relates to any one of Embodiments 8(ii), 8(ii)(a), 8(ii)(b), 8(ii)(c), 8(ii)(d), or 8(ii)(e) wherein R² is saturated 7-9 membered heterocycloalkyl comprising at least one nitrogen.

Embodiment 8(iv) relates to any one of Embodiments 8(ii), 8(ii)(a), 8(ii)(b), 8(ii)(c), 8(ii)(d), or 8(ii)(e) wherein R² is

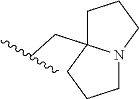 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 8(v) relates to Embodiment 8(ii) wherein when G is H, then R² is

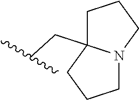 [SMILES: *CC12CCCN1CCC2]

that is substituted with 0-3 R⁴.

Embodiment 9(ii) of this disclosure relates to the compound according to any one of Embodiments 1(ii), 2(ii), 3(ii), 4(ii), 5(ii), 6(ii), 7(ii), or 8(ii), wherein R¹ is 7-9 membered bridged piperazine or piperidine substituted with 0-2 T¹ and 1 G.
Embodiment 9(ii)(a) relates to Embodiment 9(ii) wherein G is H.
Embodiment 9(ii)(b) relates to Embodiment 9(ii) wherein G is not H.
Embodiment 9(ii)(c) relates to Embodiment 9(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), heterocycloalkyl, bridged heterocycloalkyl, heterocyclic spiro ring, aryl, or heteroaryl.
Embodiment 9(ii)(d) relates to Embodiment 9(ii) wherein G has one of the following Formulae:

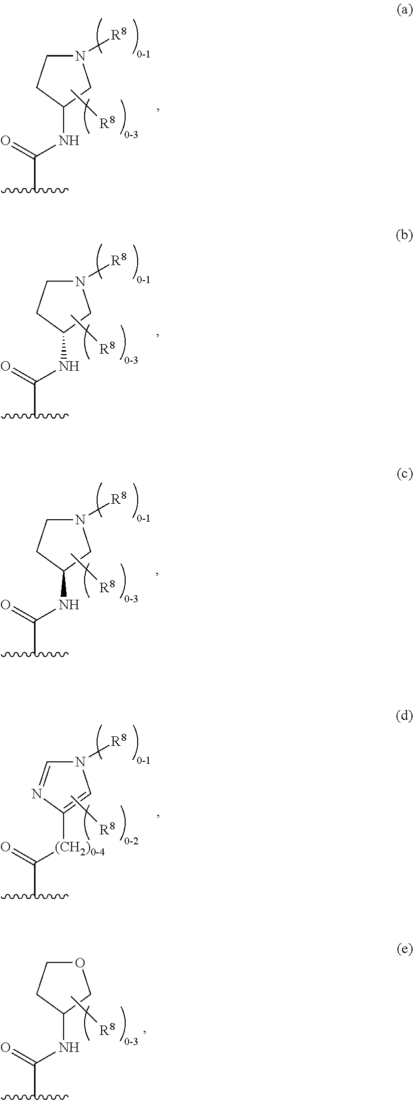

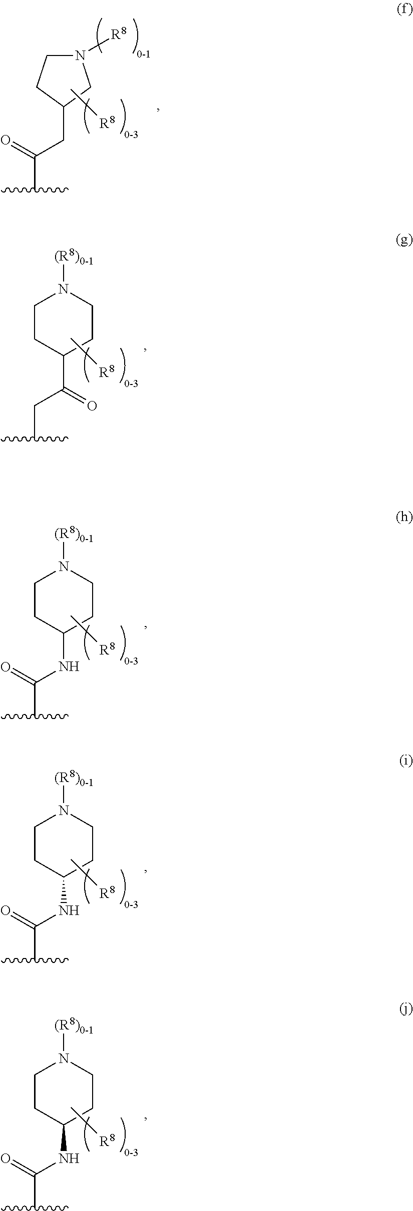

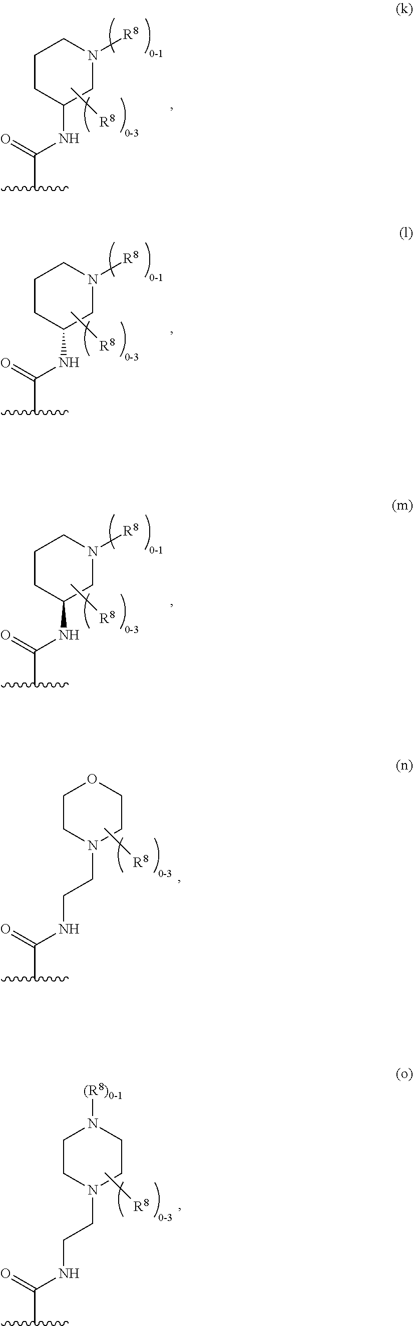

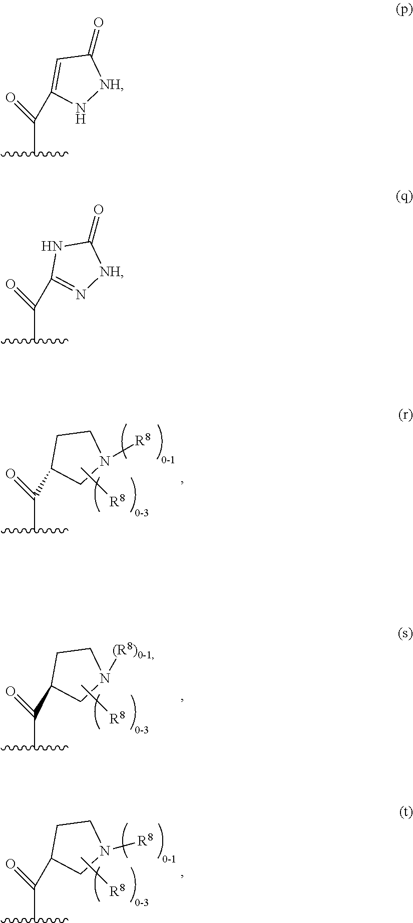

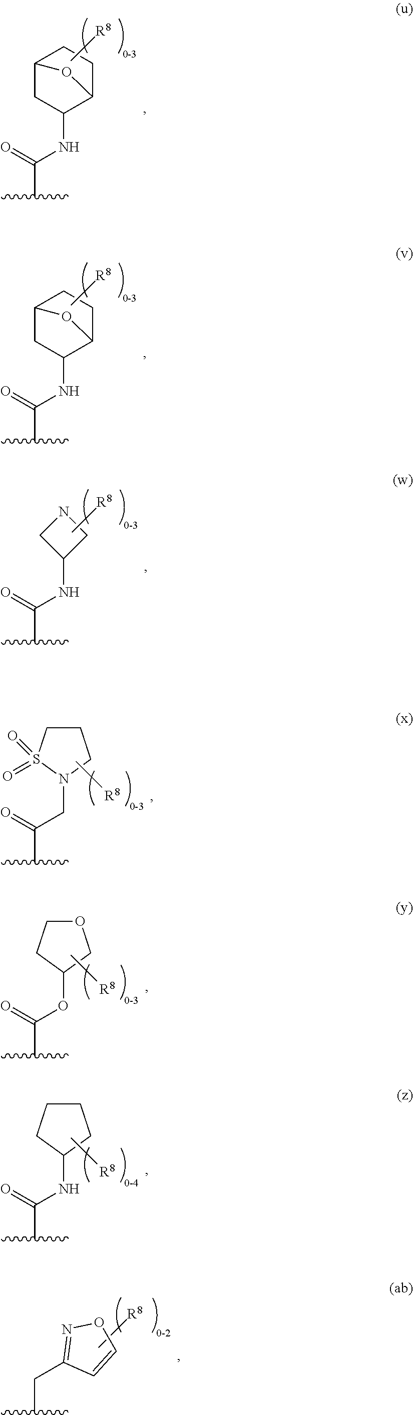

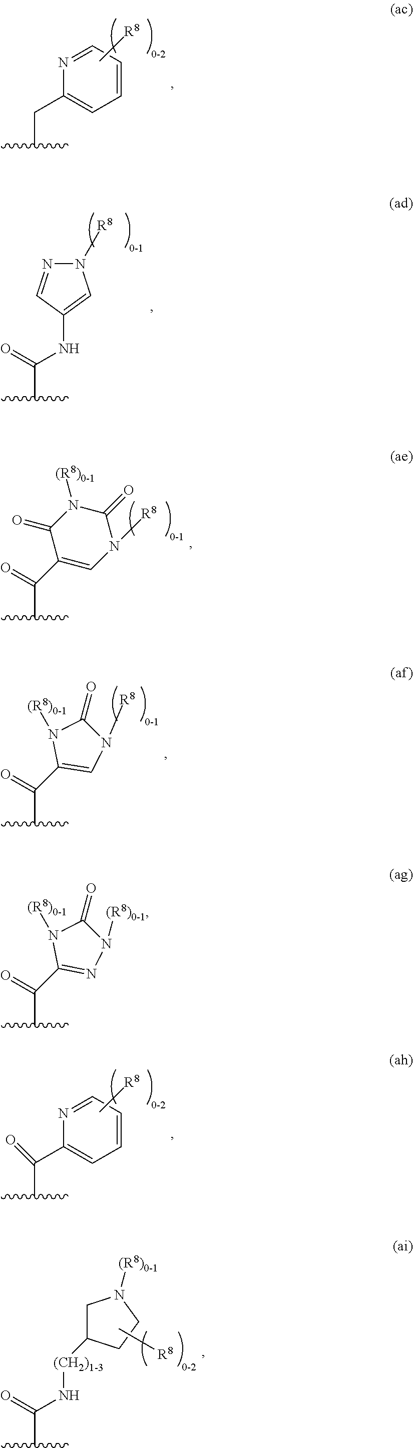

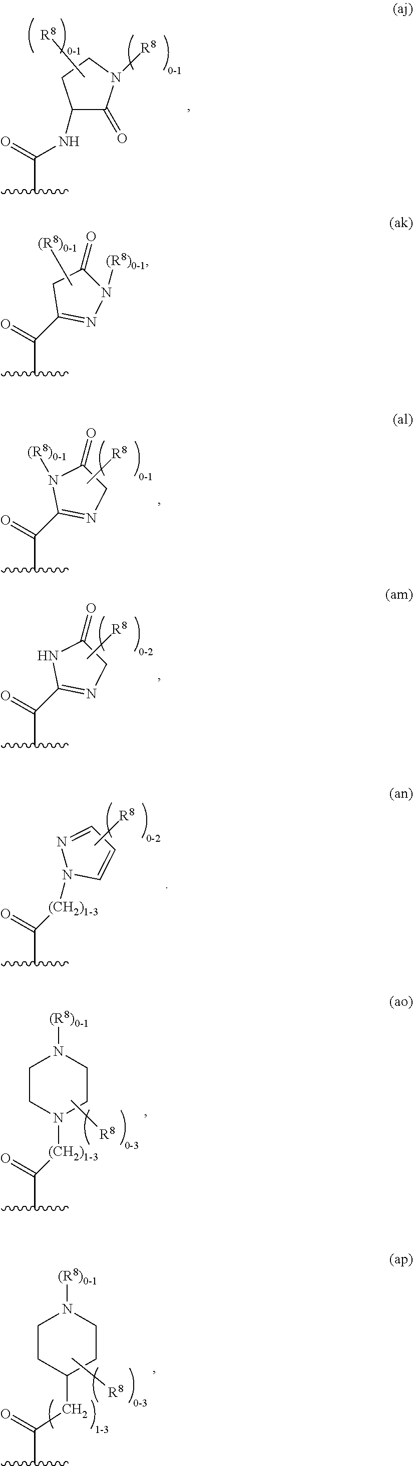

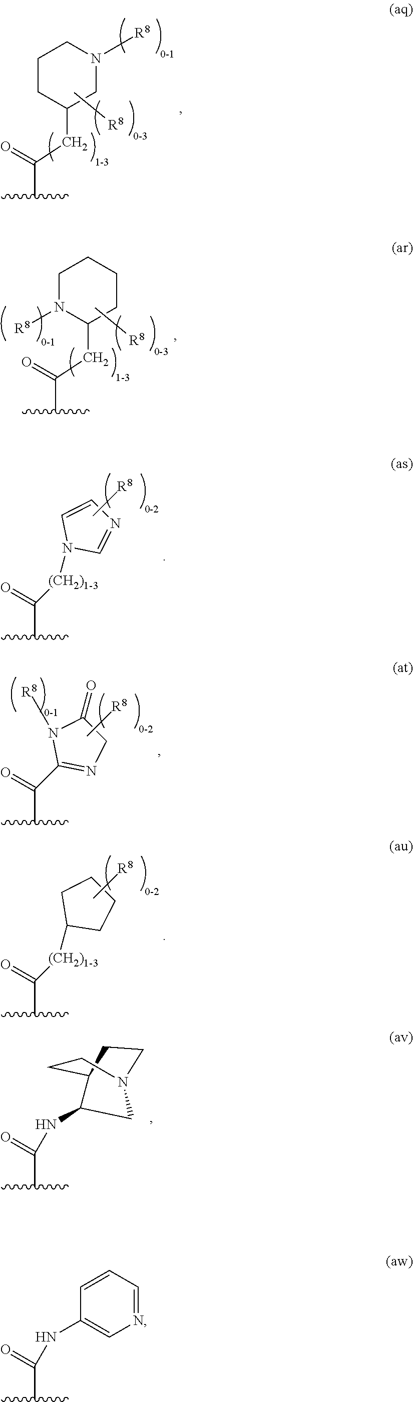

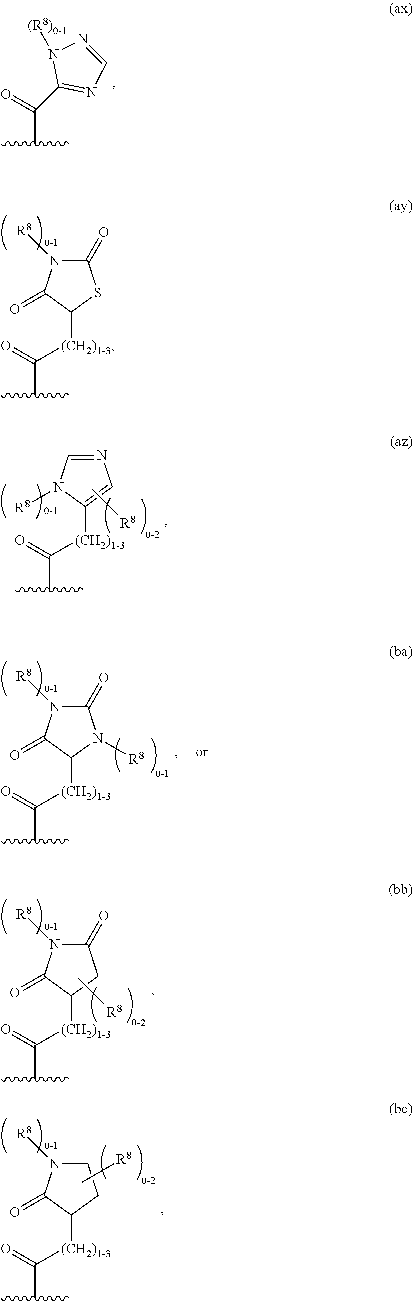

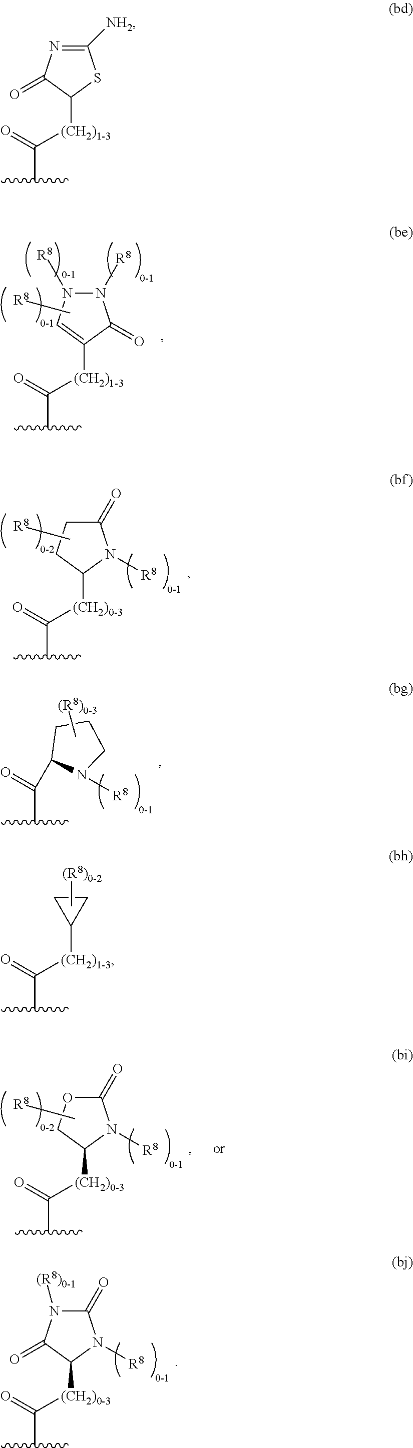

Embodiment 9(ii)(e) relates to Embodiment 9(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 9(iii) relates to any one of Embodiments 9(ii), 9(ii)(a), 9(ii)(b), 9(ii)(c), 9(ii)(d), or 9(ii)(e) wherein R² is

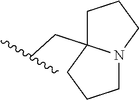 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 9(iv) relates to Embodiment 9(ii) wherein when G is H, then R² is

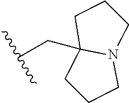 [SMILES: *CC12CCCN1CCC2]

that is substituted with 0-3 R⁴.

Embodiment 10(ii) of this disclosure relates to the compound according to any one of Embodiments 1(ii), 2(ii), 3(ii), 4(ii), 5(ii), 6(ii), 7(ii), or 8(ii), wherein R¹ is one of the following formulae:

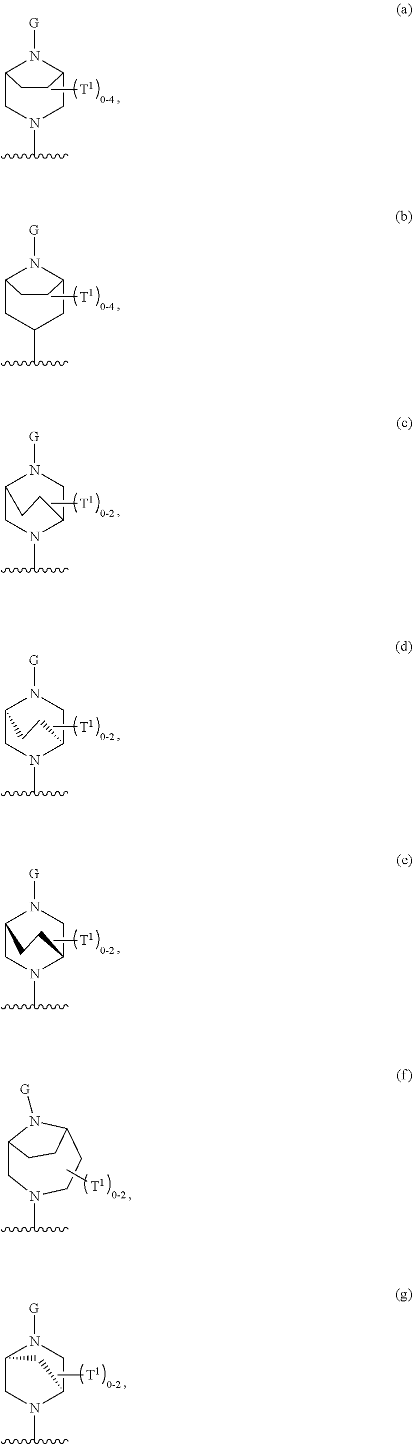

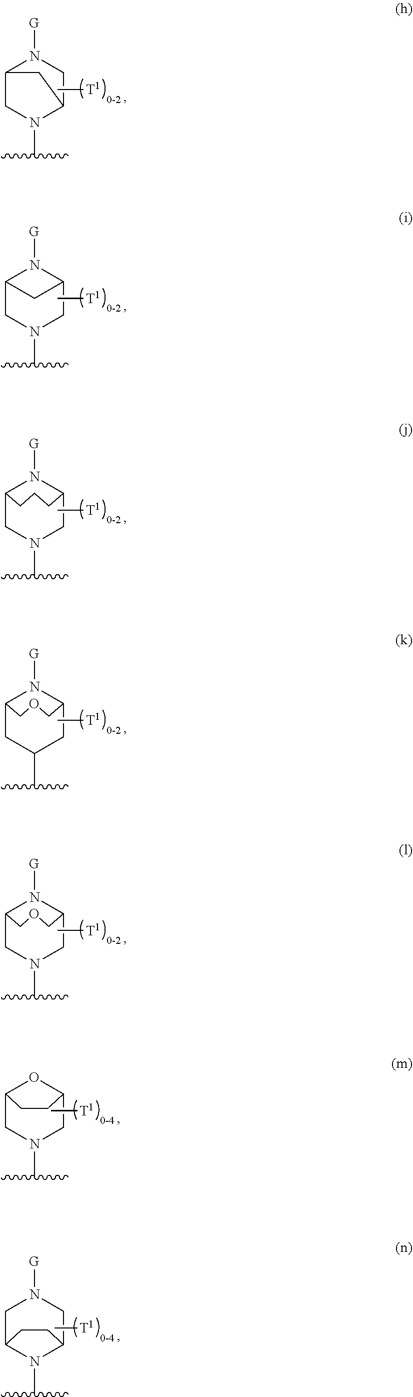

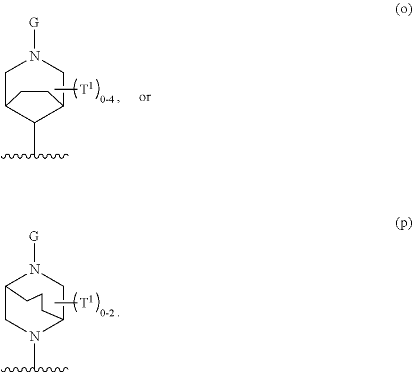

Embodiment 10(ii)(a) relates to Embodiment 10(ii) wherein G is H.
Embodiment 10(ii)(b) relates to Embodiment 10(ii) wherein G is not H.
Embodiment 10(ii)(c) relates to Embodiment 10(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 10(ii)(d) relates to Embodiment 10(ii) wherein G has one of the following Formulae:

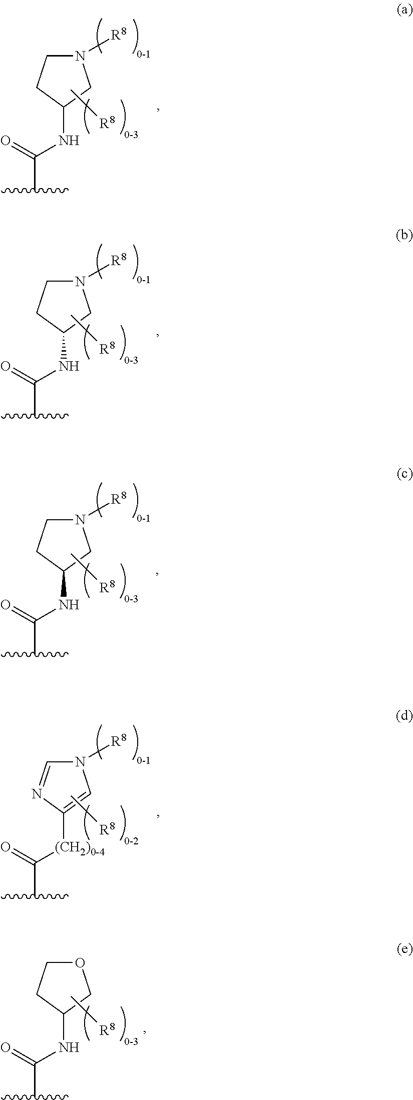

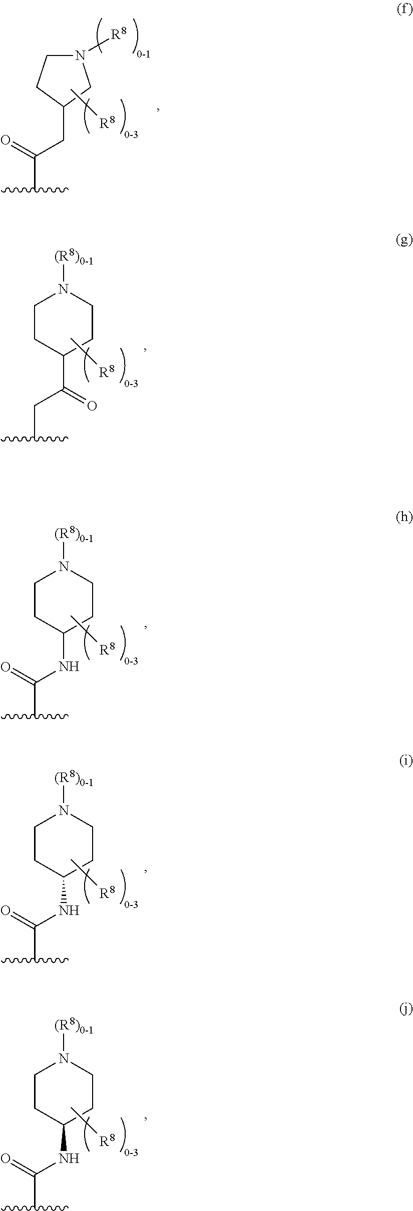

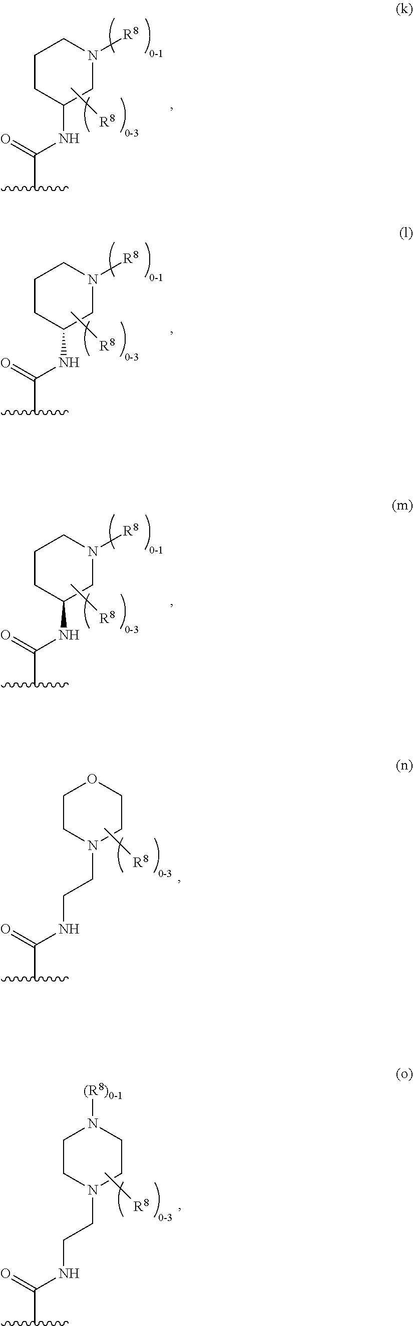

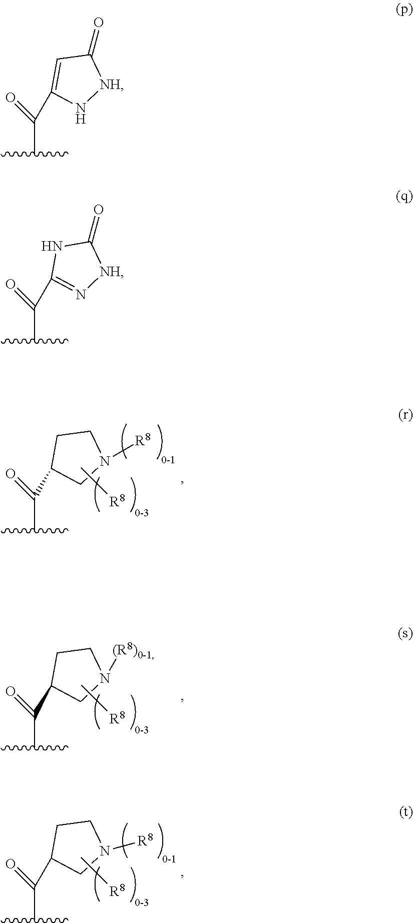

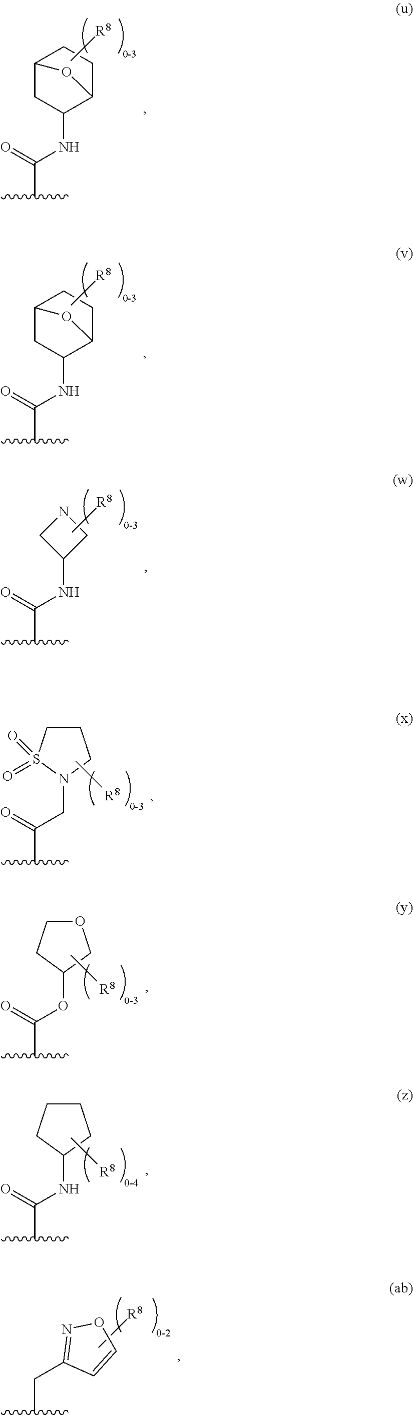

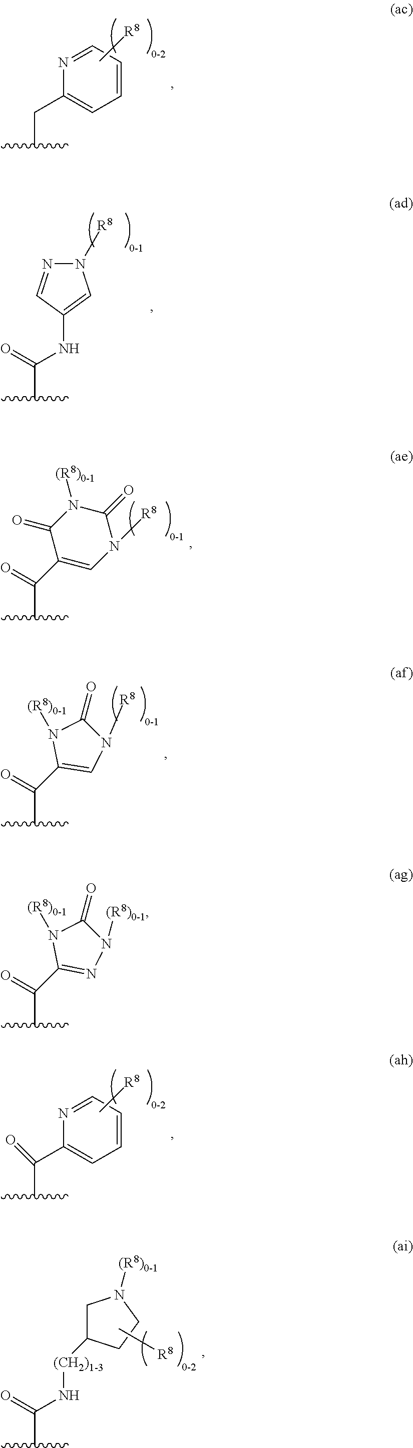

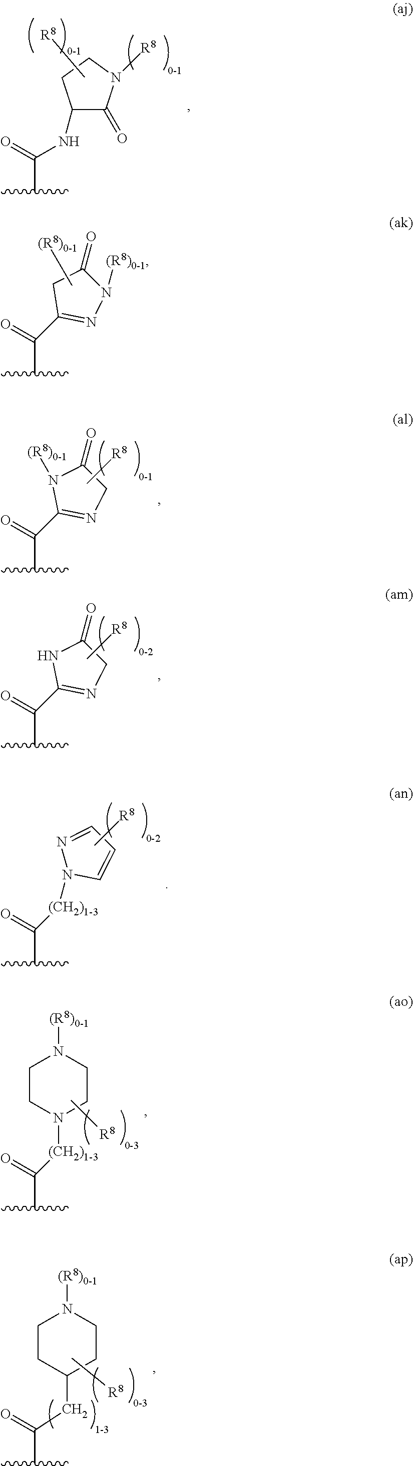

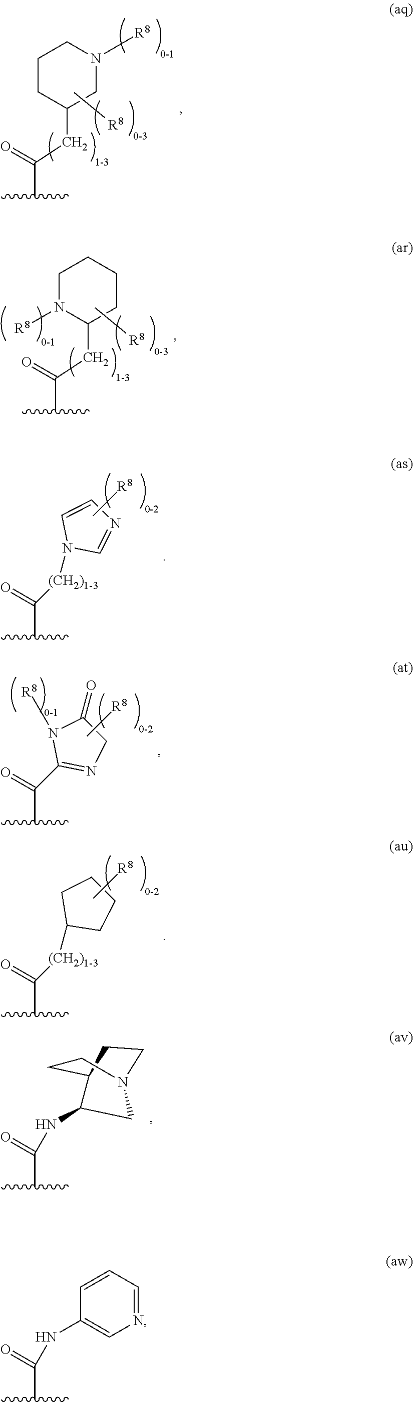

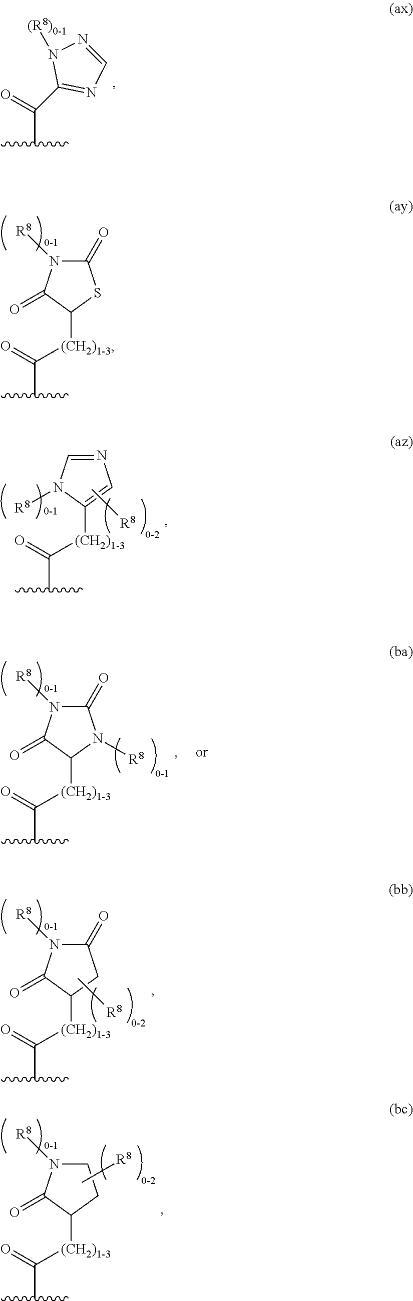

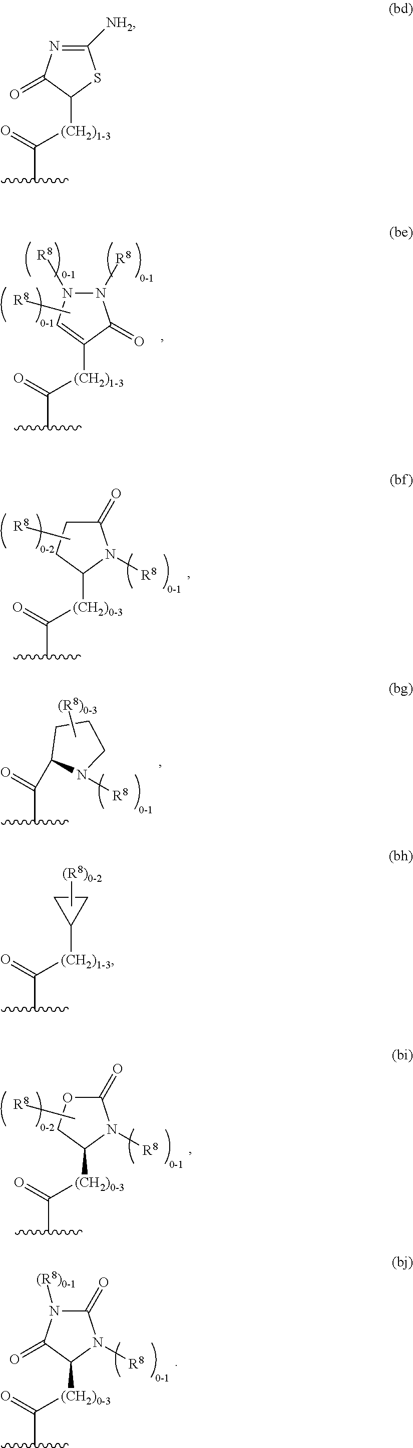

Embodiment 10(ii)(e) relates to Embodiment 10(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 10(iii) relates to any one of Embodiments 10(ii), 10(ii)(a), 10(ii)(b), 10(ii)(c), 10(ii)(d), or 10(ii)(e) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 10(iv) relates to any one of Embodiments 10(ii), 10(ii)(a), 10(ii)(b), 10(ii)(c), 10(ii)(d), or 10(ii)(e) wherein R² is saturated 7-9 membered heterocycloalkyl comprising at least one nitrogen.
Embodiment 10(v) relates to any one of Embodiments 10(ii), 10(ii)(a), 10(ii)(b), 10(ii)(c), 10(ii)(d), or 10(ii)(e) wherein
R² is

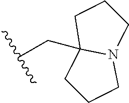 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 11 (ii) of this disclosure relates to the compound according to Embodiment 10(ii), wherein R¹ is formula (a) or (b).
Embodiment 11(ii)(a) relates to Embodiment 11(ii) wherein G is H.
Embodiment 11(ii)(b) relates to Embodiment 11(ii) wherein G is not H.

Embodiment 11(iii) relates to Embodiment 11(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 11(iv) relates to any one of Embodiments 11(ii), 11(ii)(a), or 11(ii)(b) wherein R² is saturated 7-9 membered heterocycloalkyl comprising at least one nitrogen.

Embodiment 11(v) relates to any one of Embodiments 11(ii), 11(ii)(a), or 11(ii)(b) wherein R² is

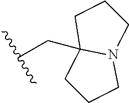 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 11 (vi) relates to the compound of Embodiment 11 (ii) wherein R¹ has the Formula (a).

Embodiment 11(vii) relates to Embodiment 1(ii) wherein when G is H, then R² is

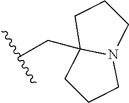 [SMILES: *CC12CCCN1CCC2]

that is substituted with 0-3 R⁴.

Embodiment 12(ii) of this disclosure relates to the compound according to any one of Embodiments 1(ii), 2(ii), 3(ii), 4(ii), or 5(ii), wherein R² is one of the following formulae:

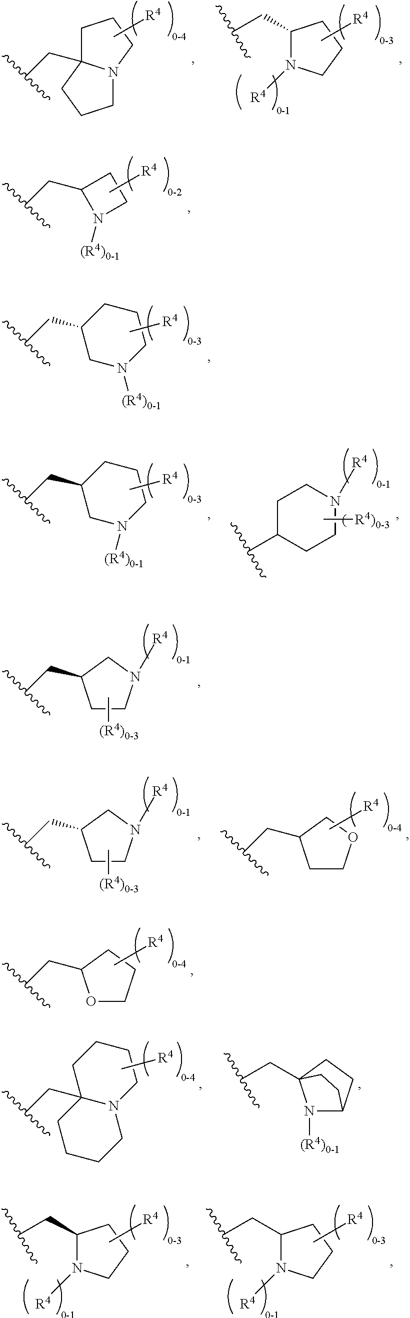

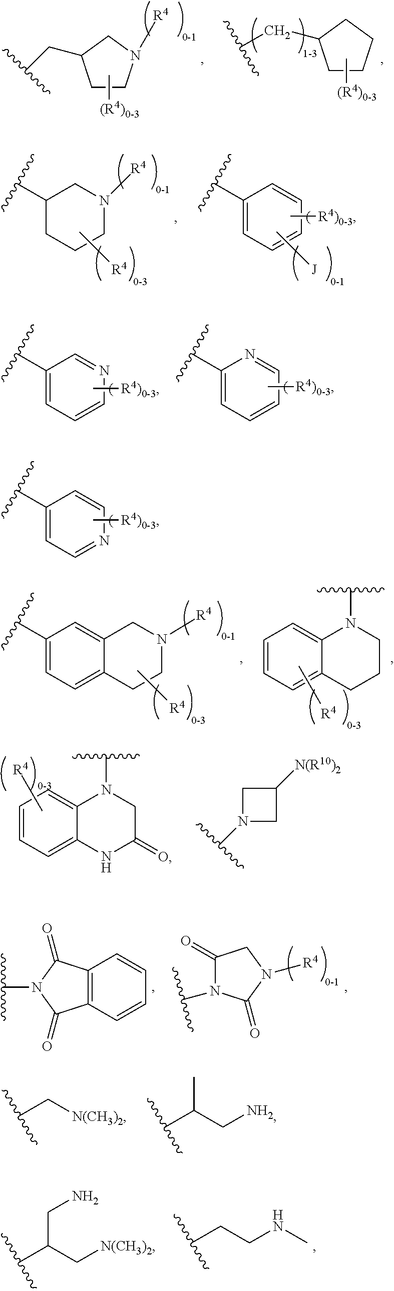

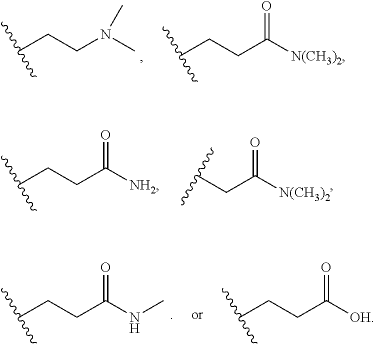

Embodiment 12(ii)(a) relates to Embodiment 12(ii) wherein G is H.
Embodiment 12(ii)(b) relates to Embodiment 12(ii) wherein G is not H.
Embodiment 12(ii)(c) relates to Embodiment 12(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 12(ii)(d) relates to Embodiment 12(ii) wherein G has one of the following Formulae:

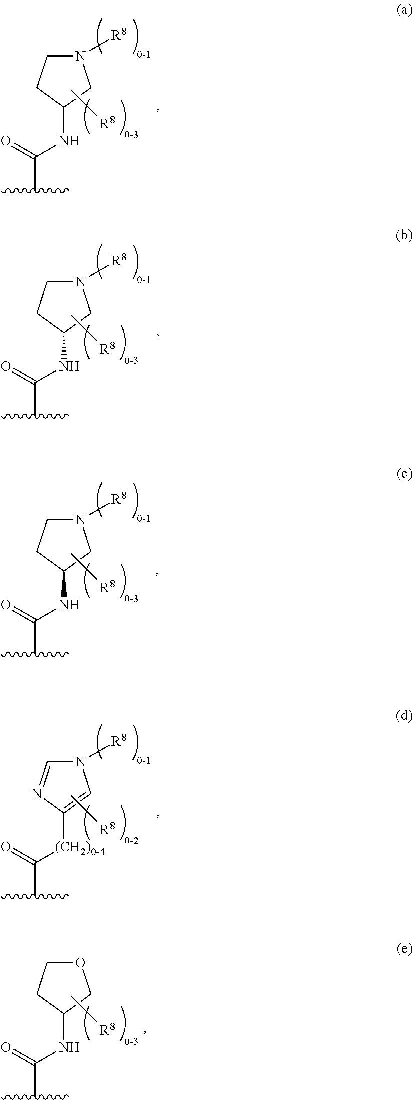

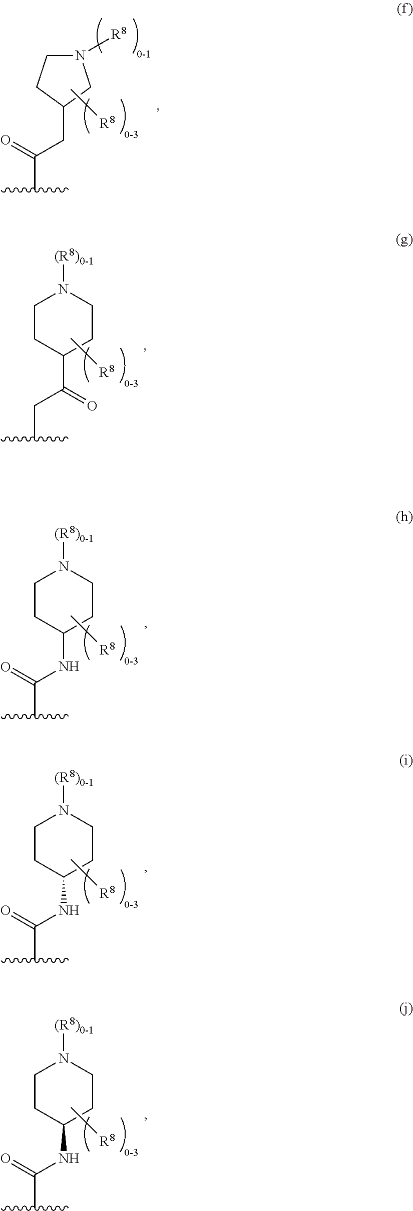

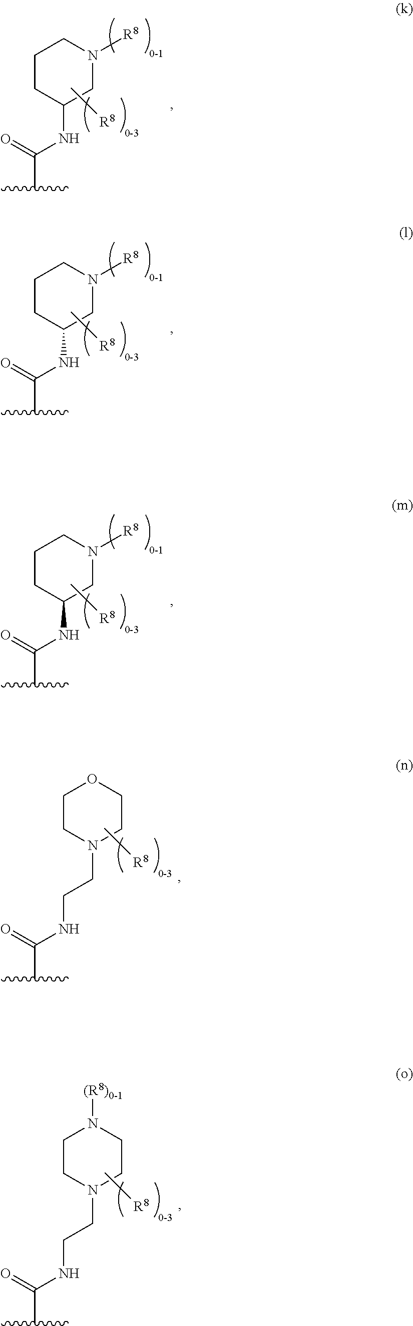

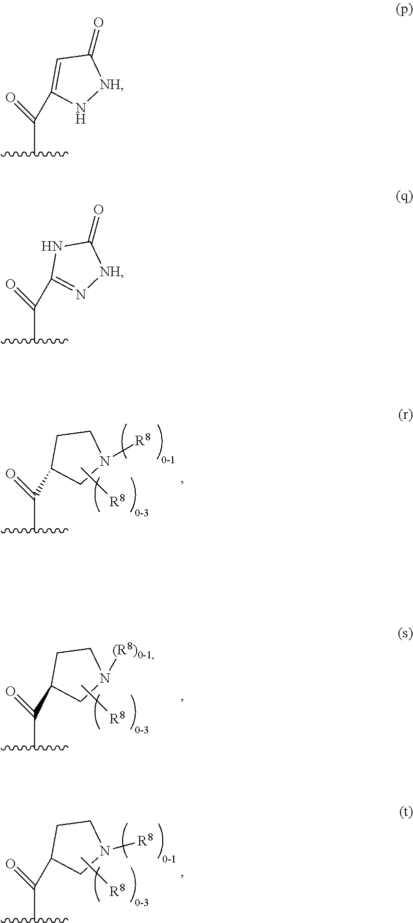

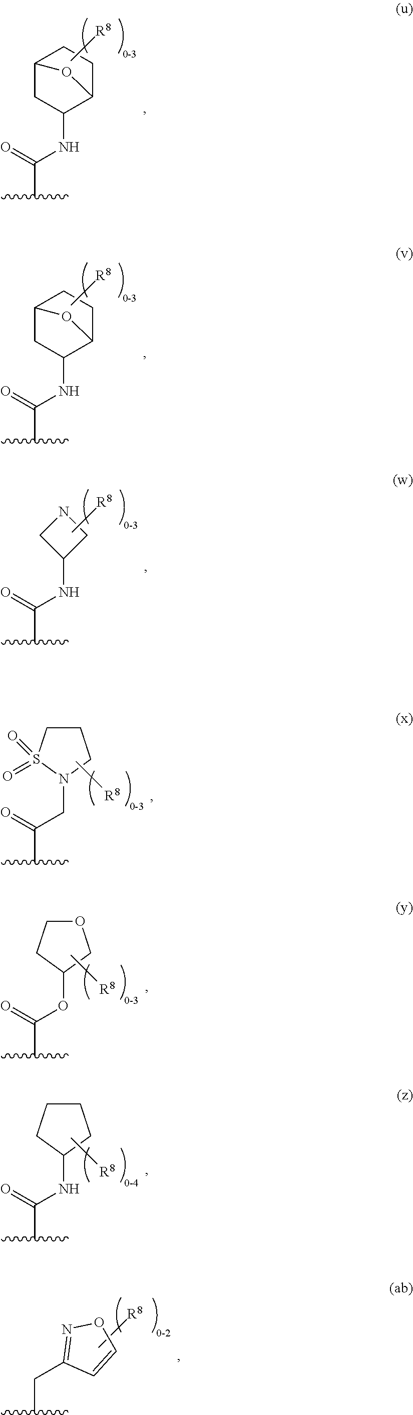

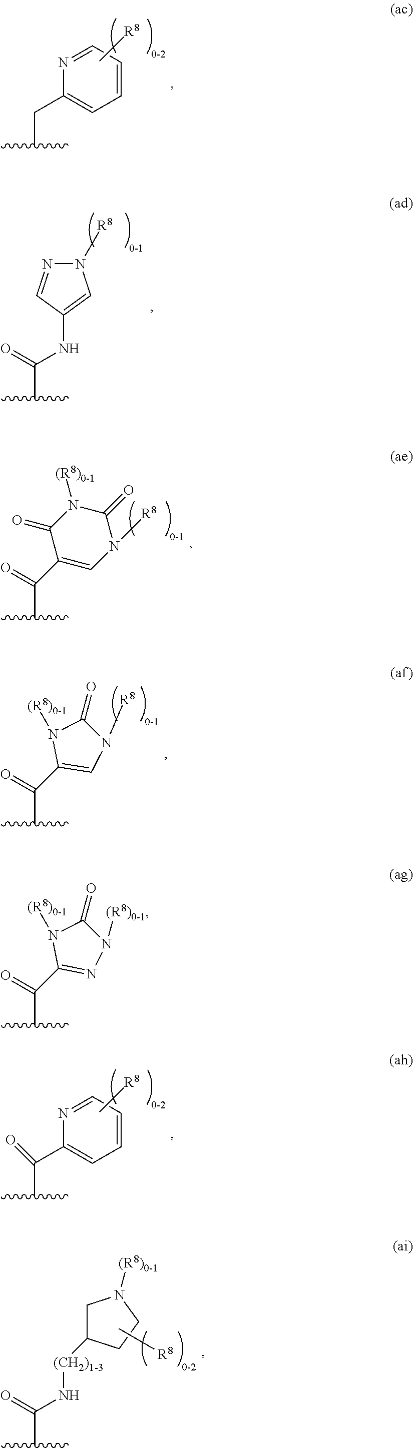

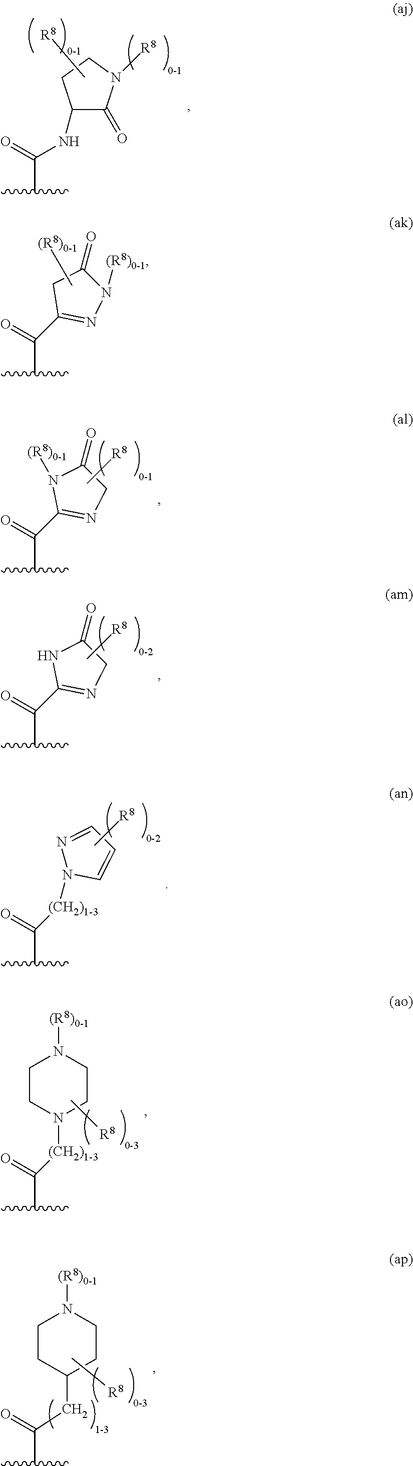

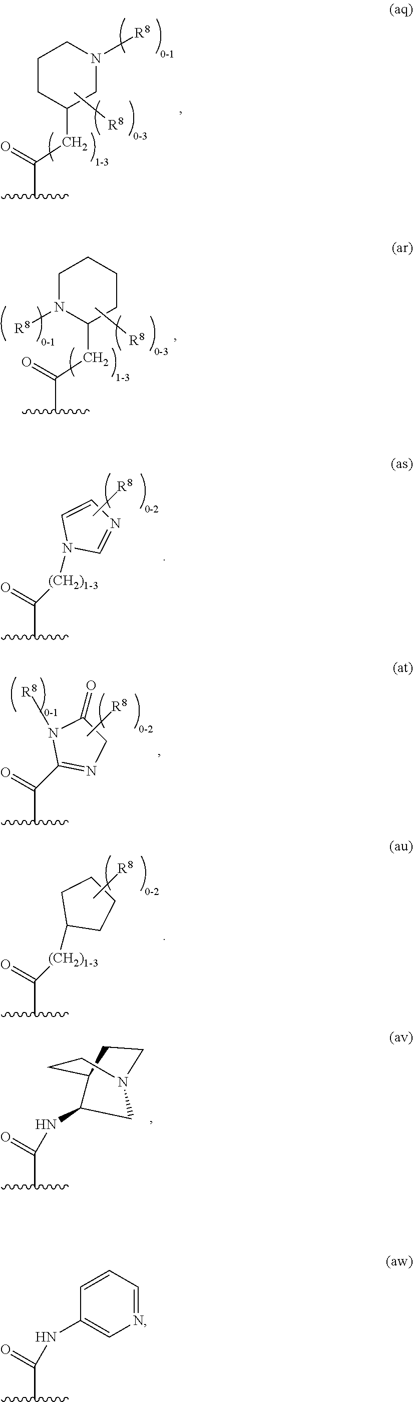

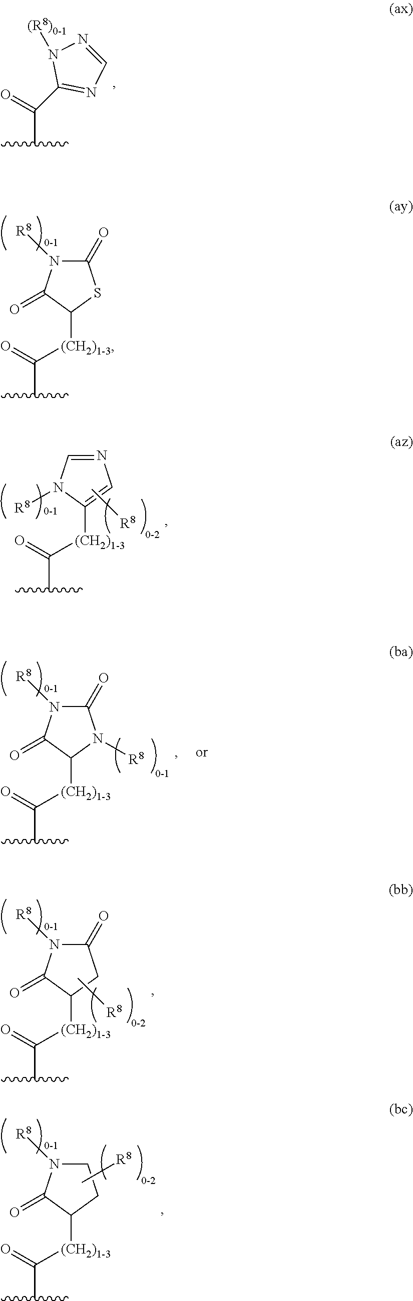

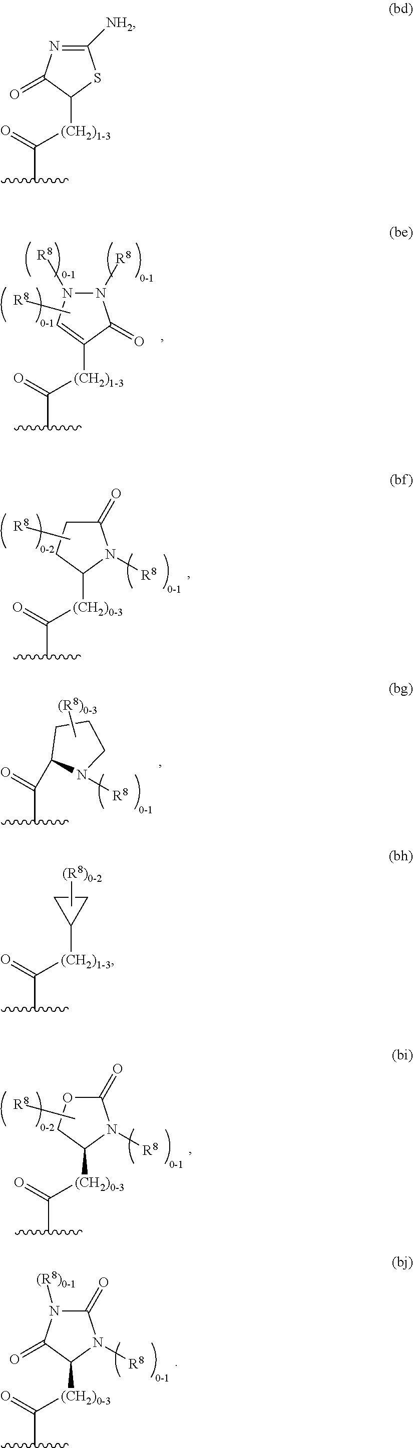

Embodiment 12(ii)(e) relates to Embodiment 12(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 12(iii) relates to Embodiment 12(ii) wherein when G is H, then R² is

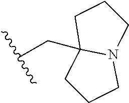 [SMILES: *CC12CCCN1CCC2]

that is substituted with 0-3 R⁴.

Embodiment 13(ii) of this disclosure relates to the compound according to any of 1(ii), 2(ii), 3(ii), 4(ii), 5(ii), 6(ii), 7(ii), 8(ii), 9(ii), 10(ii), 11(ii), or 12(ii), wherein R² is one of the following formulae and R⁴ is H, Cl, or F:

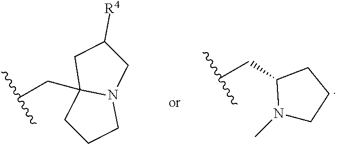

Embodiment 13(ii)(a) relates to Embodiment 13(ii) wherein G is H.
Embodiment 13(ii)(b) relates to Embodiment 13(ii) wherein G is not H.
Embodiment 13(ii)(c) relates to Embodiment 13(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 13(ii)(d) relates to Embodiment 13(ii) wherein G has one of the following Formulae:

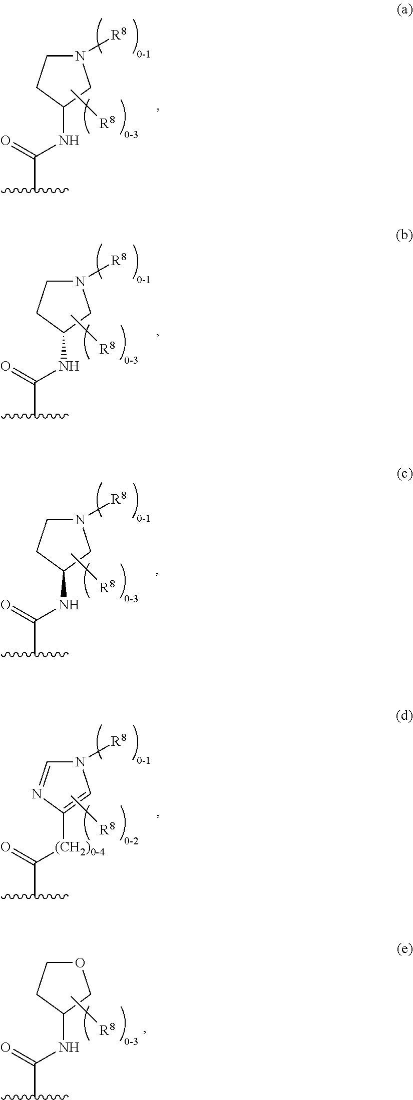

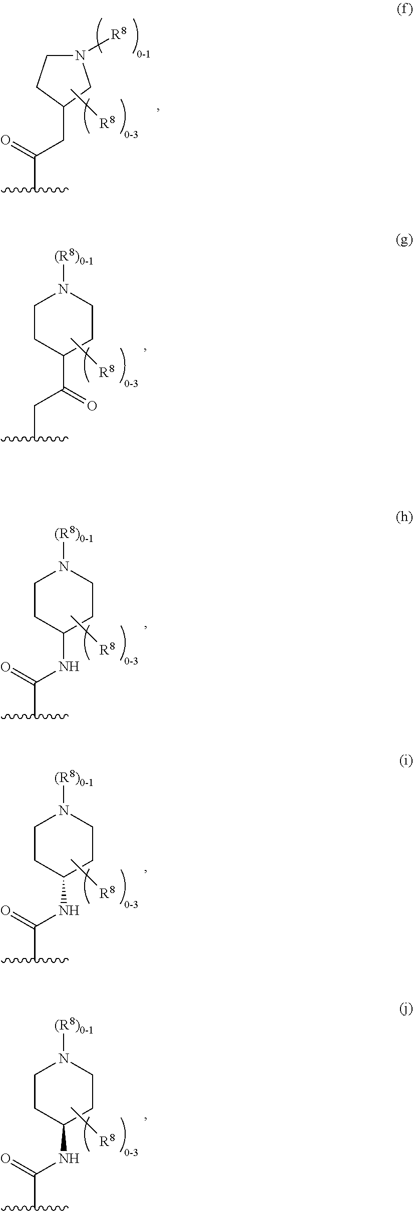

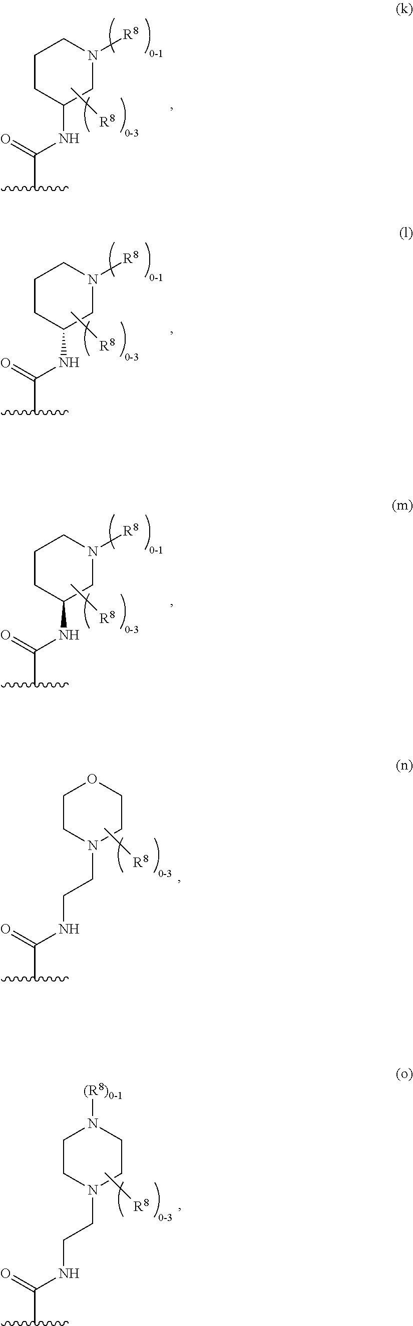

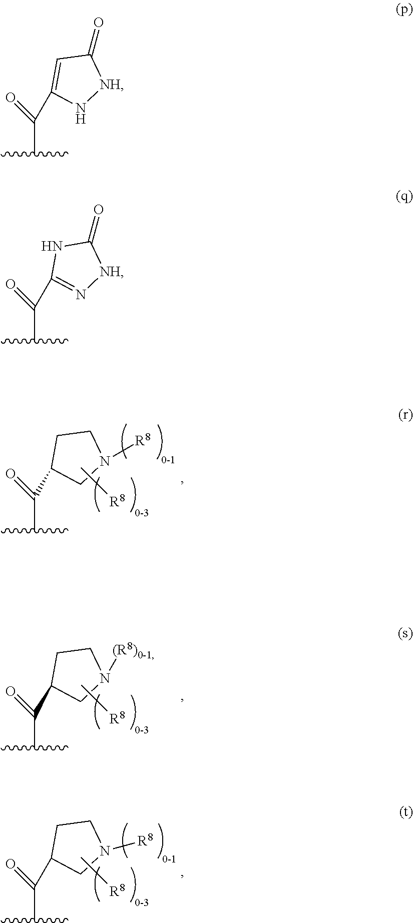

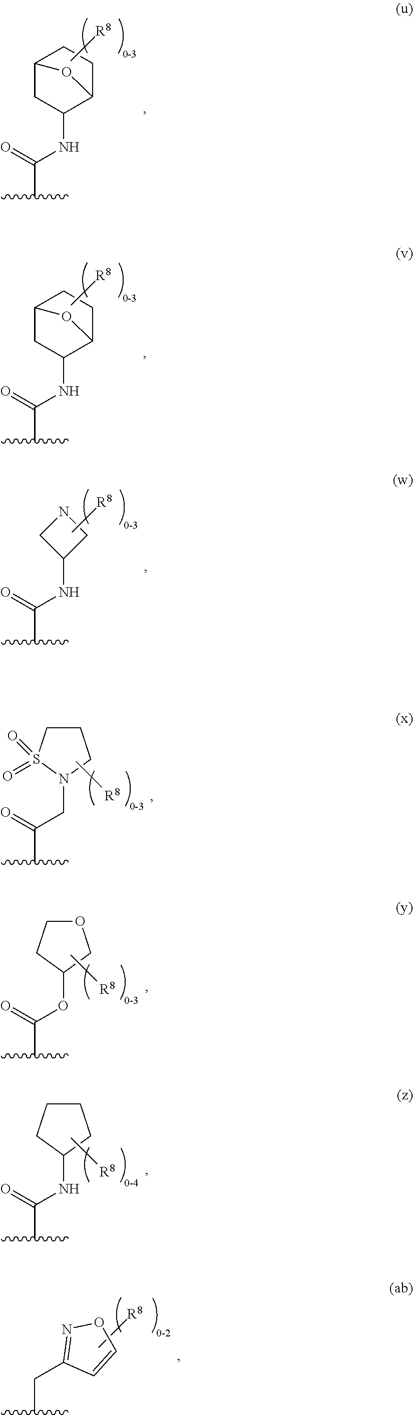

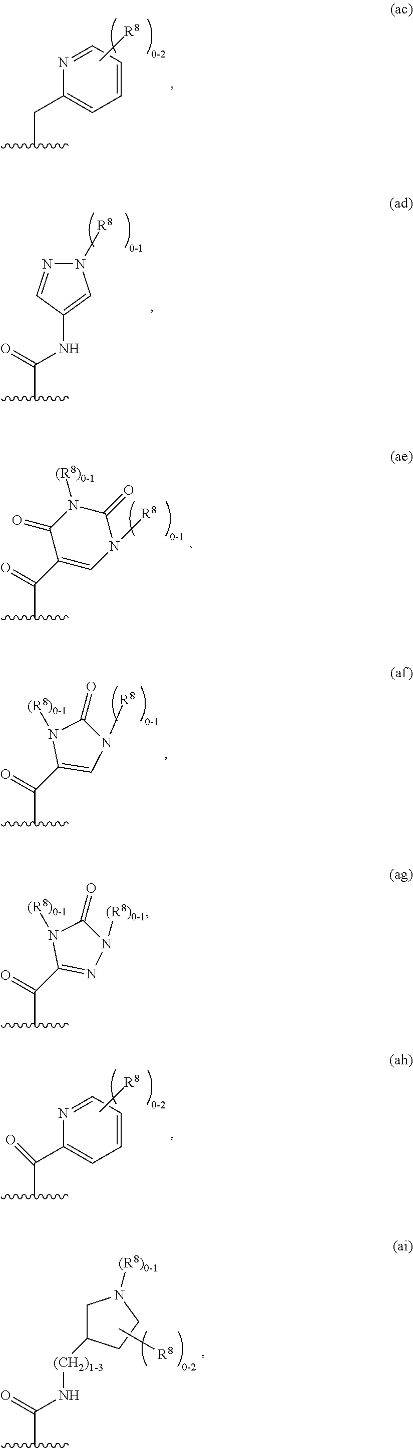

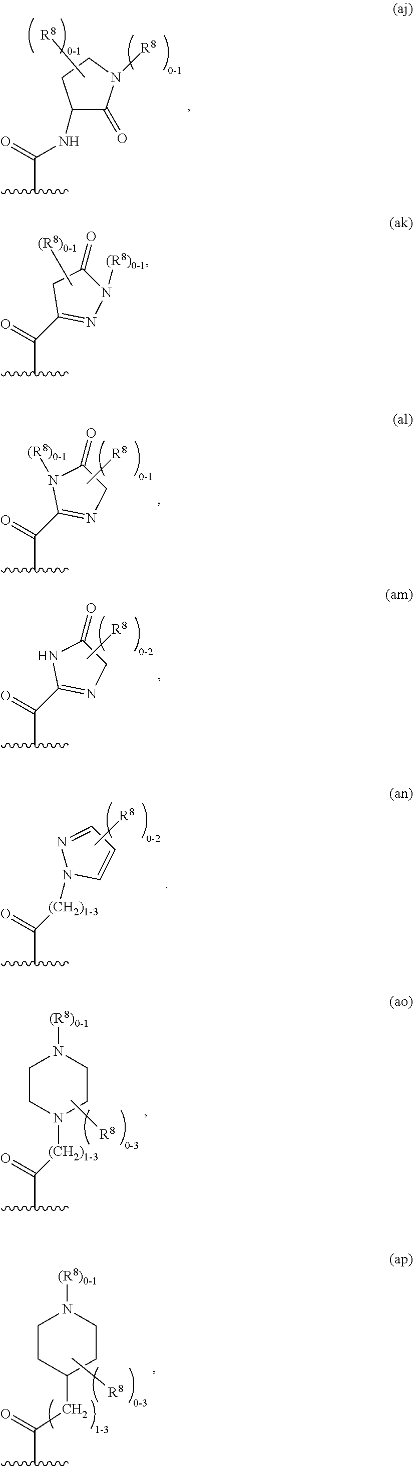

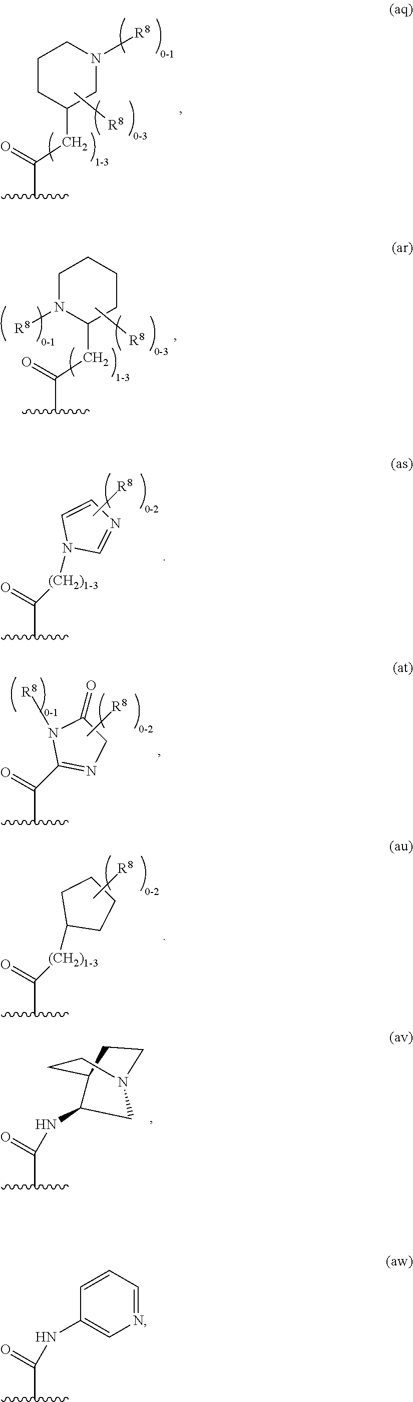

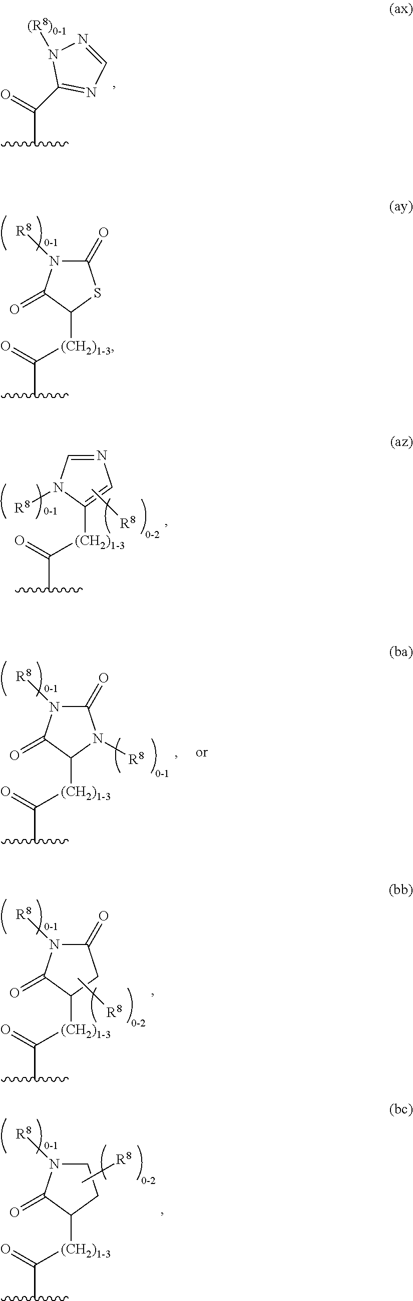

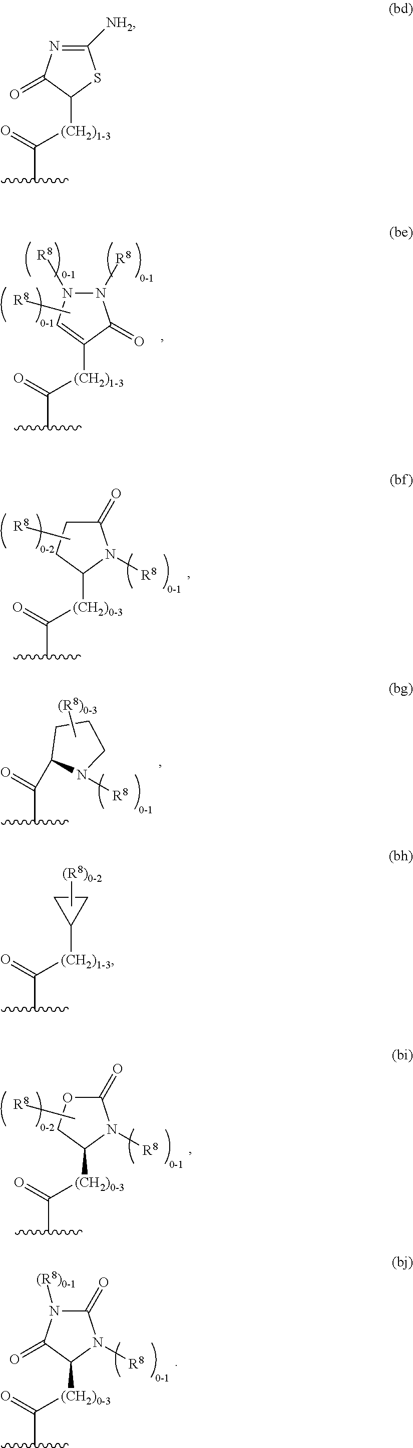

Embodiment 13(ii)(e) relates to Embodiment 13(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 13(iii) relates to the compound of Embodiment 13(ii) wherein R⁴ is H and R² has the following formula:

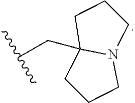 [SMILES: *CC12CCCN1CCC2]

Embodiment 13(iv) relates to the compound of Embodiment 13(ii) wherein R⁴ is F and R² has the following formula:

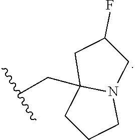 [SMILES: *CC12CCCN1CC(F)C2]

Embodiment 13(v) relates to the compound of Embodiment 13(ii) wherein R⁴ is Cl and R² has the following formula:

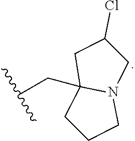 [SMILES: *CC12CCCN1CC(Cl)C2]

Embodiment 14(ii) of this disclosure relates to the compound according to any one of Embodiments 1(ii), 2(ii), 3(ii), 4(ii), 5(ii), 6(ii), 7(ii), 8(ii), 9(ii), 10(ii), 11(ii), 12(ii), or 13(h), wherein R³ is one of the following formulae:

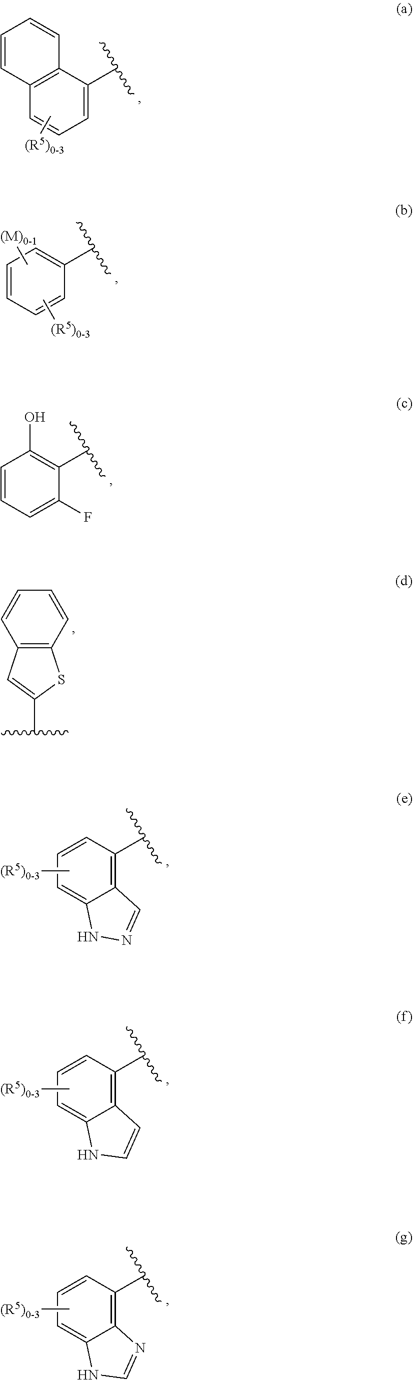

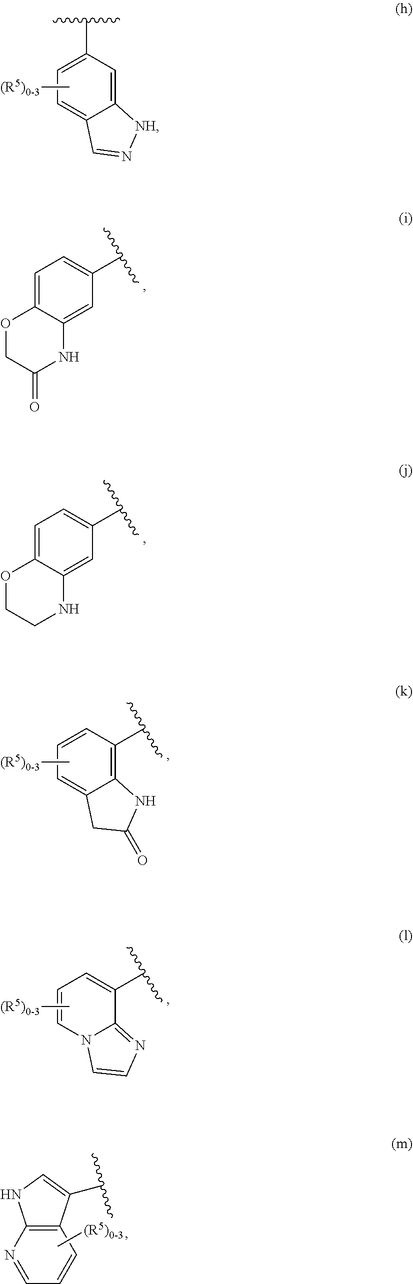

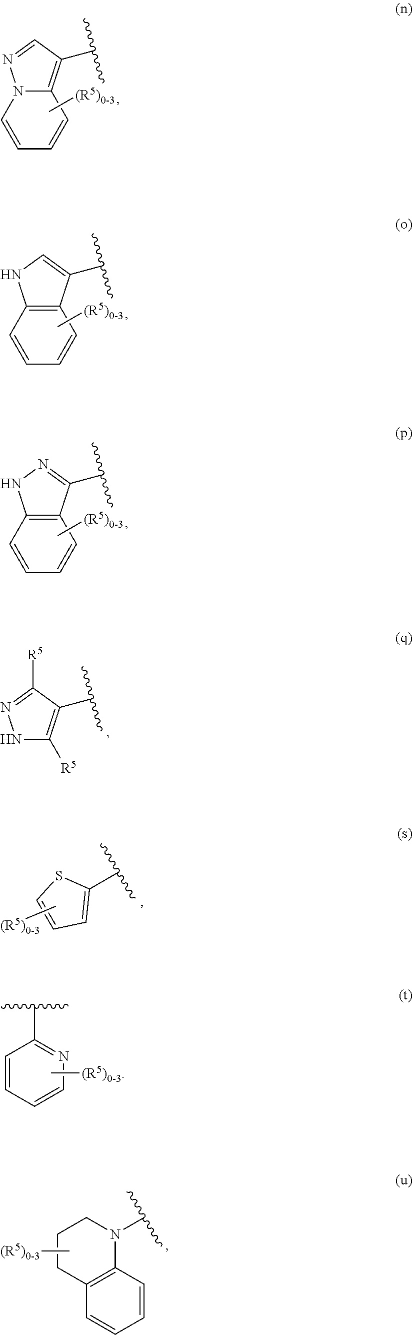

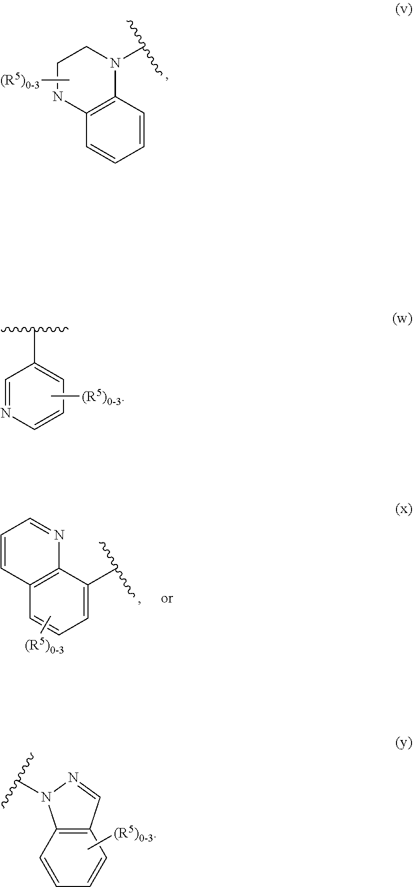

Embodiment 14(ii)(a) relates to Embodiment 14(ii) wherein G is H.
Embodiment 14(ii)(b) relates to Embodiment 14(ii) wherein G is not H.
Embodiment 14(ii)(c) relates to Embodiment 14(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 14(ii)(d) relates to Embodiment 14(ii) wherein G has one of the following Formulae:

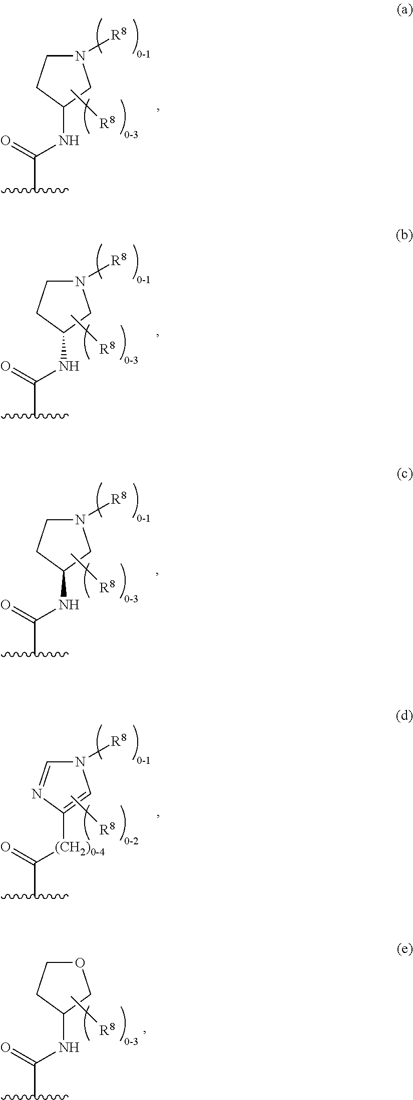

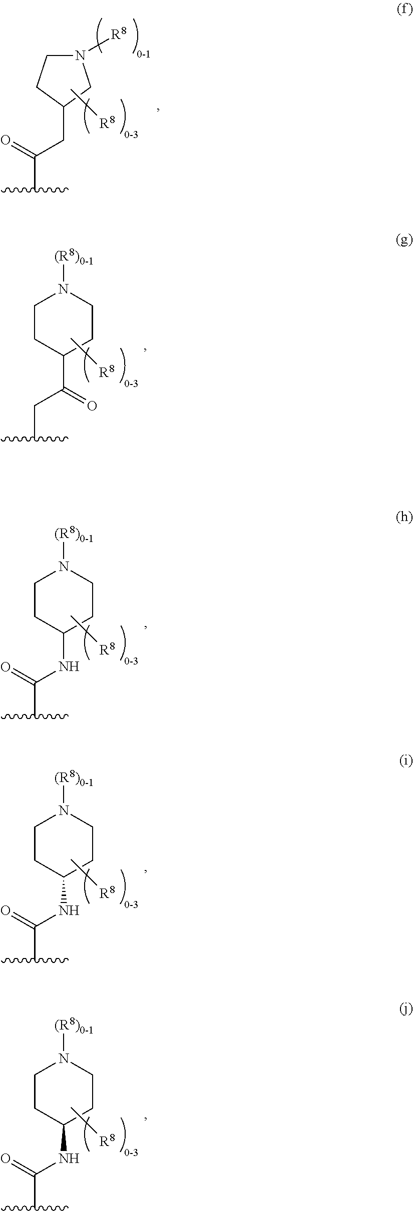

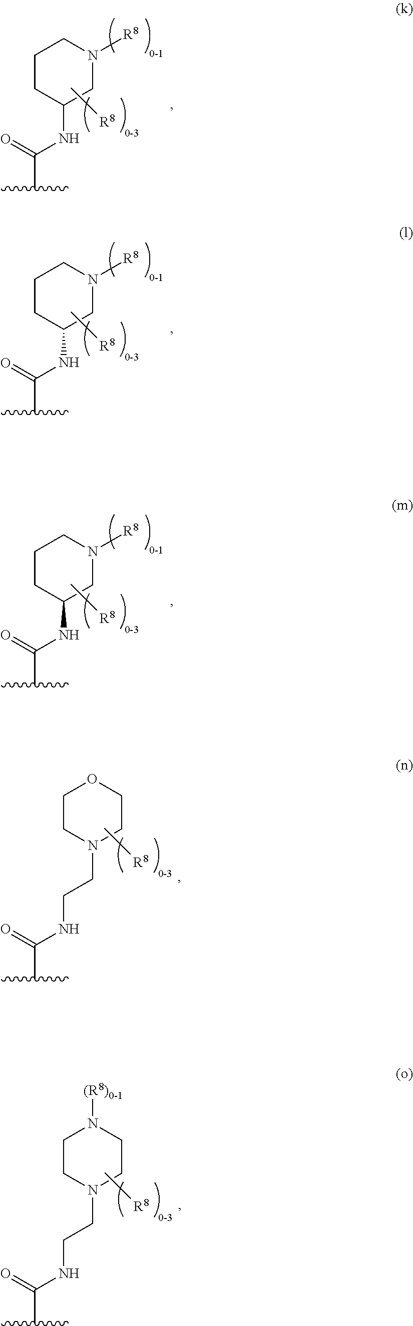

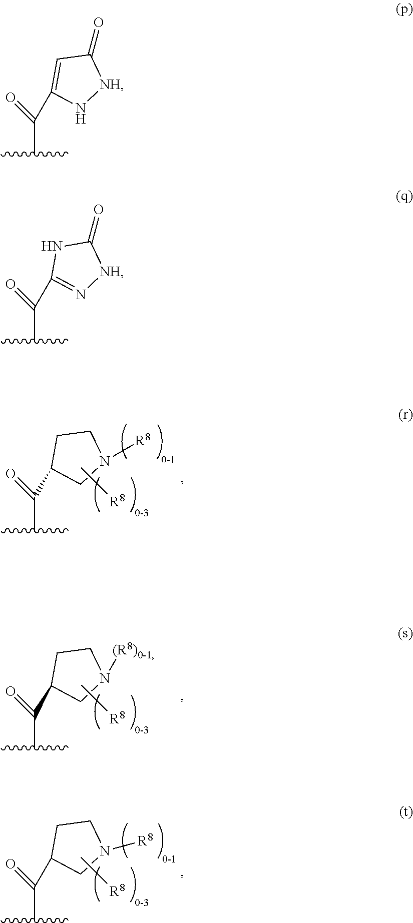

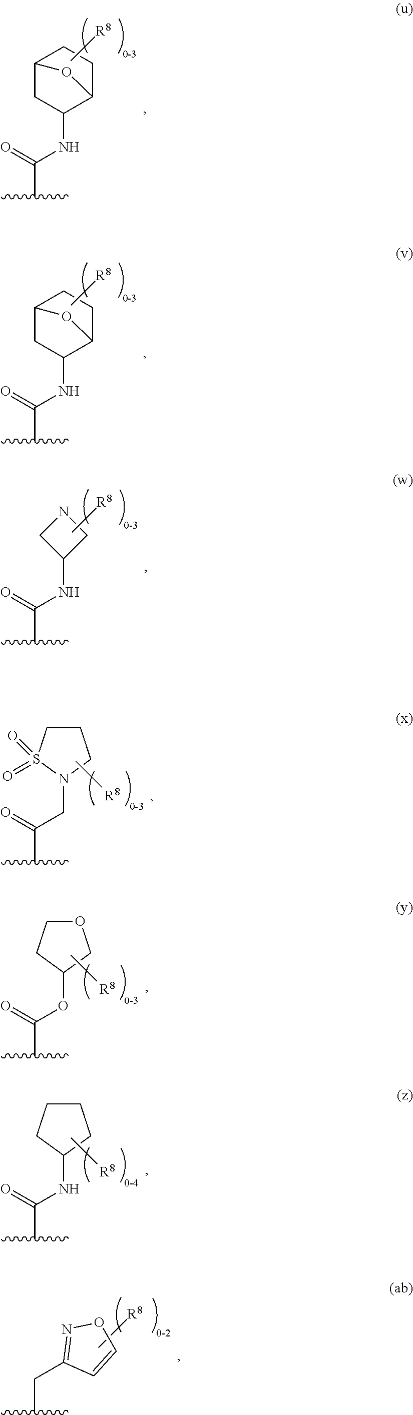

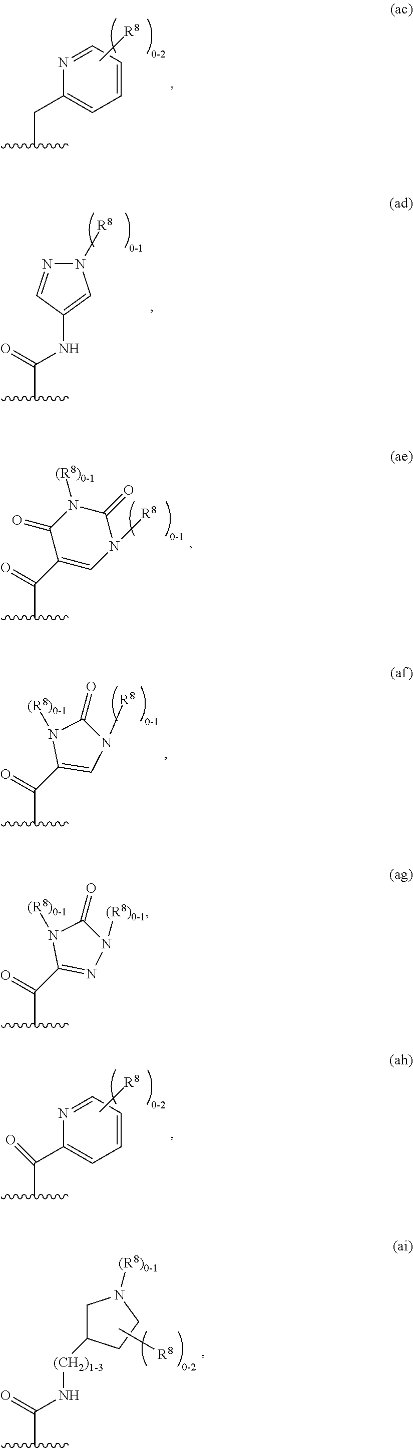

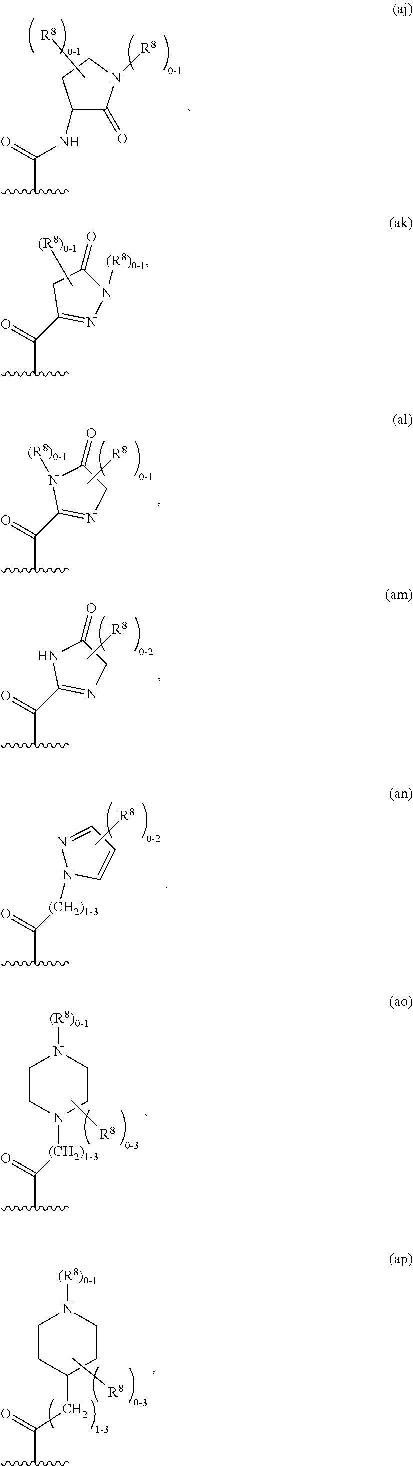

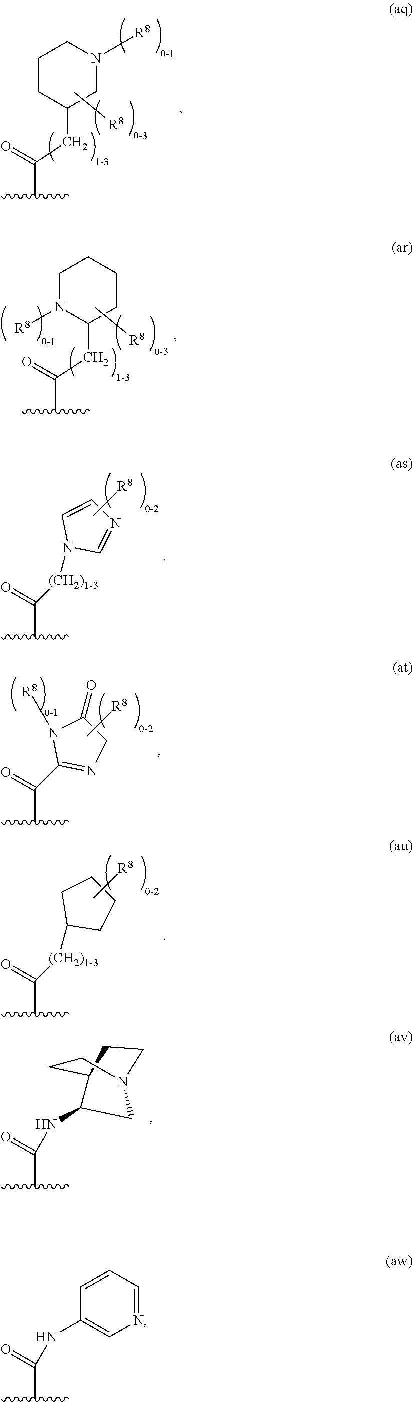

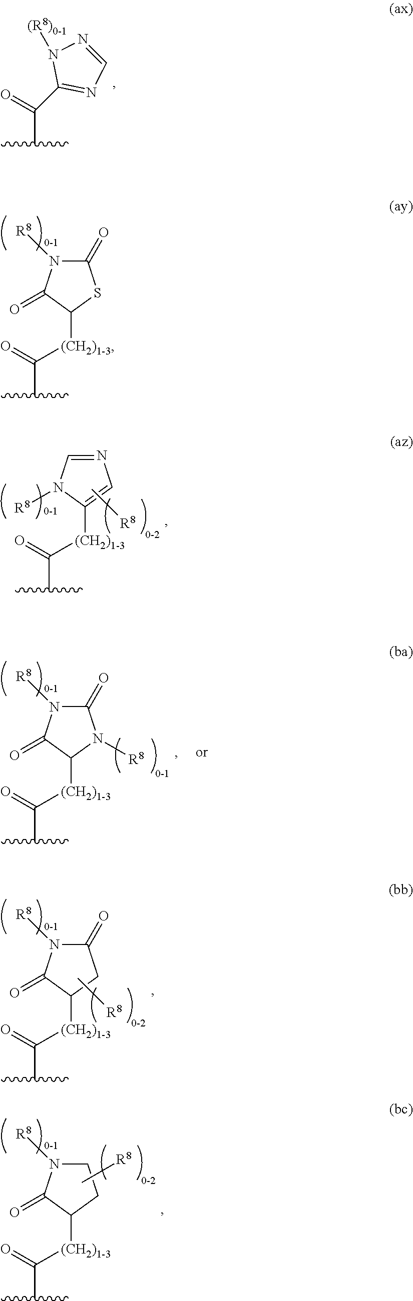

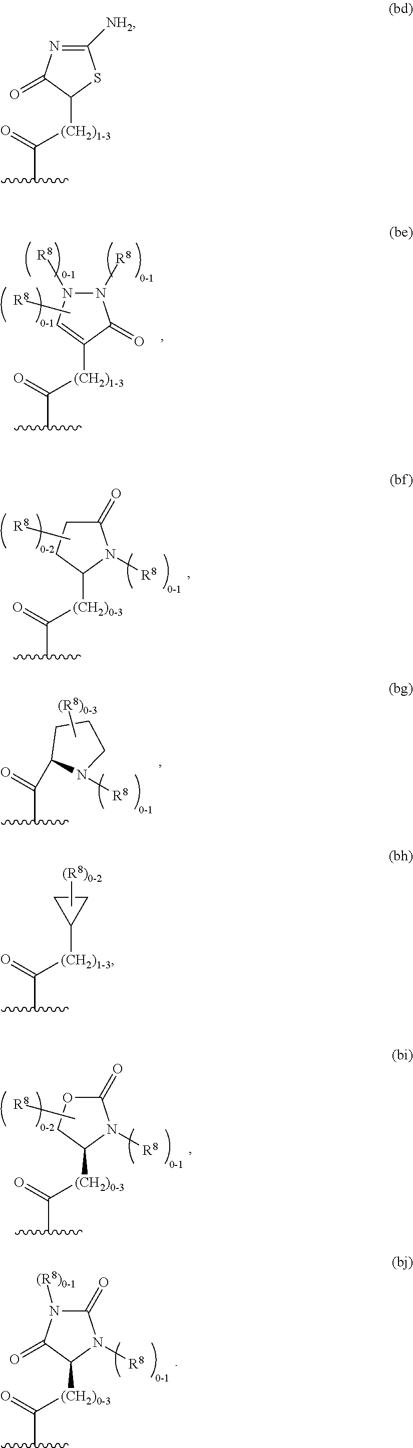

Embodiment 14(ii)(e) relates to Embodiment 14(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 14(iii) relates to Embodiment 14(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 14(iv) relates to Embodiment 14(ii) wherein R² is saturated 7-9 membered heterocycloalkyl comprising at least one nitrogen.

Embodiment 14(v) relates to Embodiment 14(ii) wherein R² is

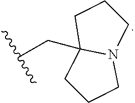 [SMILES: *CC12CCCN1CCC2]

Embodiment 15(ii) of this disclosure relates to the compound according to Embodiments 1(ii), 2(ii), 3(ii), 4(ii), 5(ii), 6(ii), 7(ii), 8(ii), 9(ii), 10(ii), 11(ii), 12(ii), 13(ii), or 14(ii), wherein R³ is

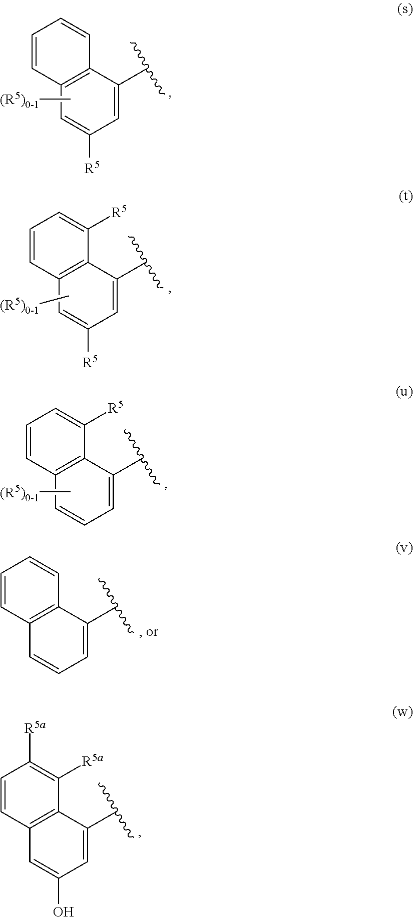

and R^{5a} is hydrogen, halo, or ethynyl.
Embodiment 15(ii)(a) relates to Embodiment 15(ii) wherein G is H.
Embodiment 15(ii)(b) relates to Embodiment 15(ii) wherein G is not H.
Embodiment 15(ii)(c) relates to Embodiment 15(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 15(ii)(d) relates to Embodiment 15(ii) wherein G has one of the following Formulae:

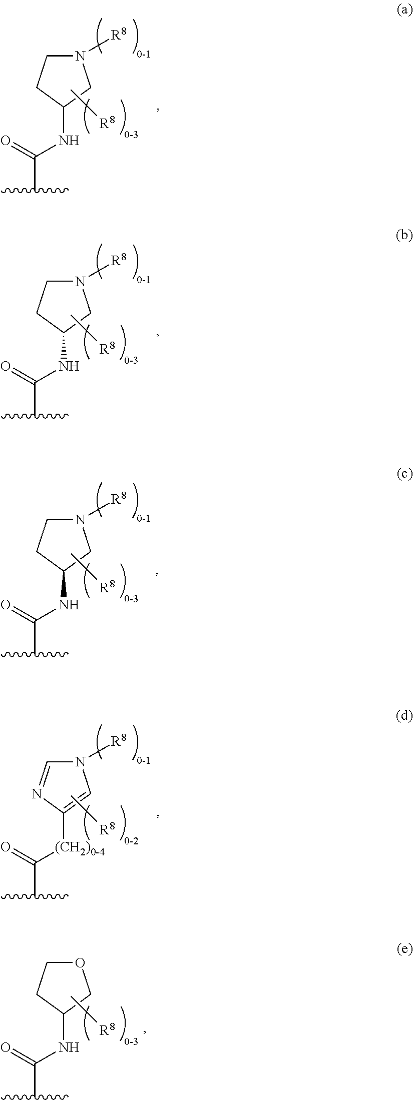

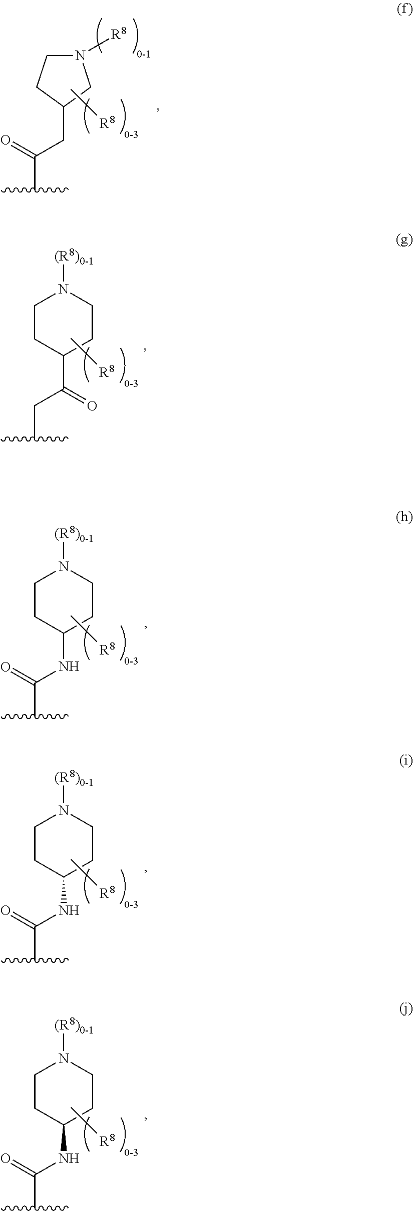

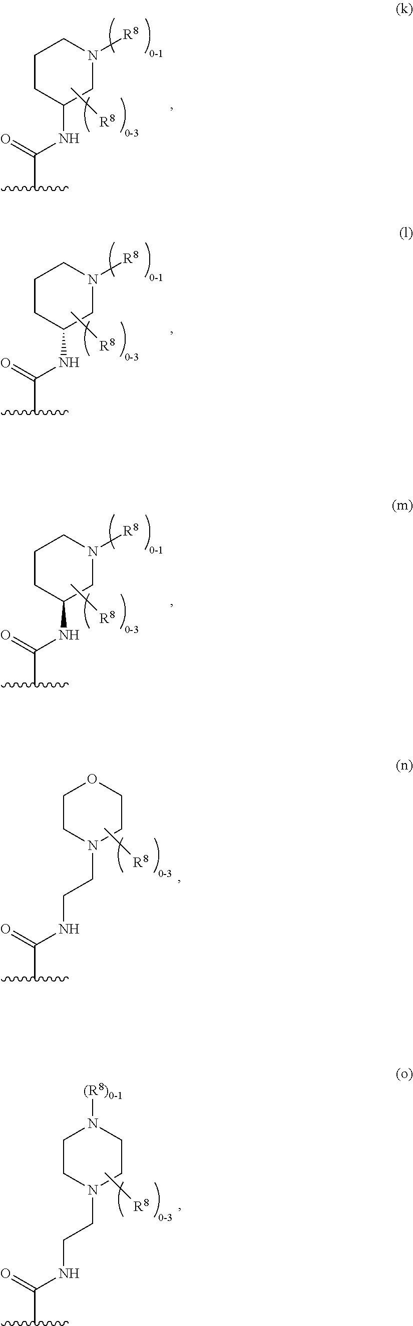

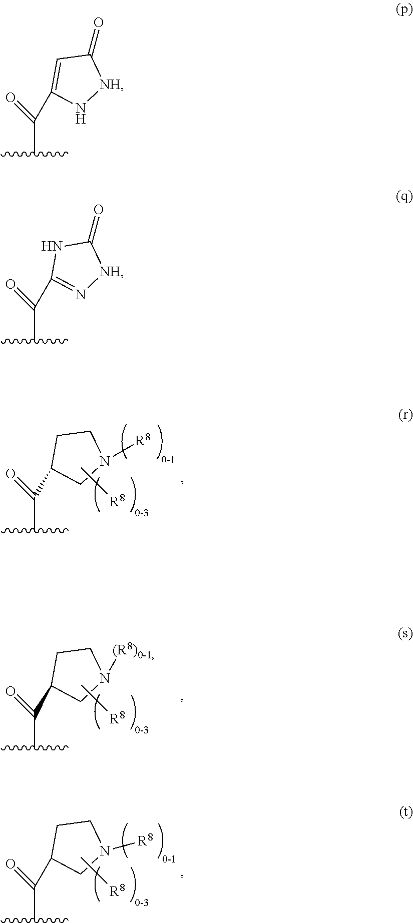

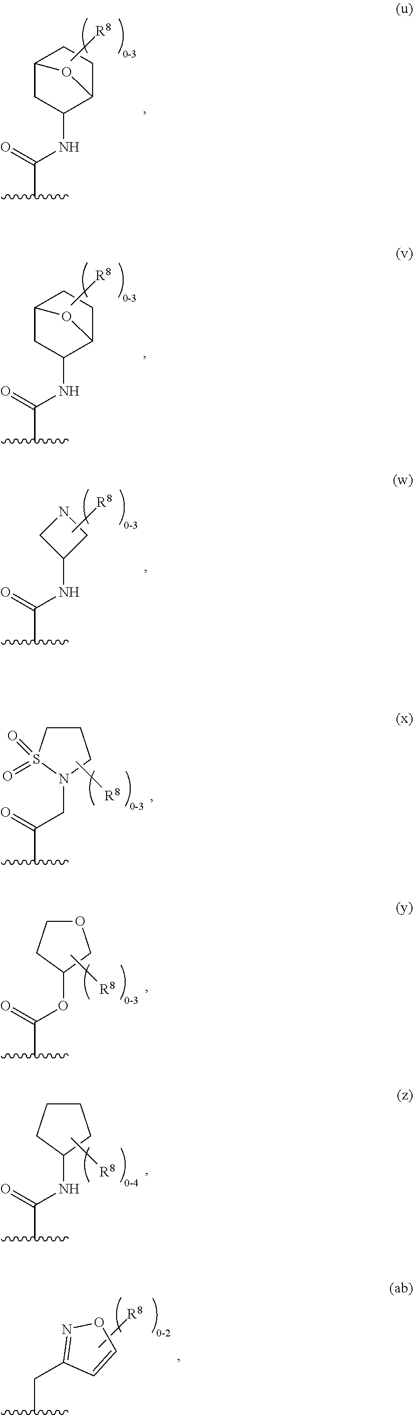

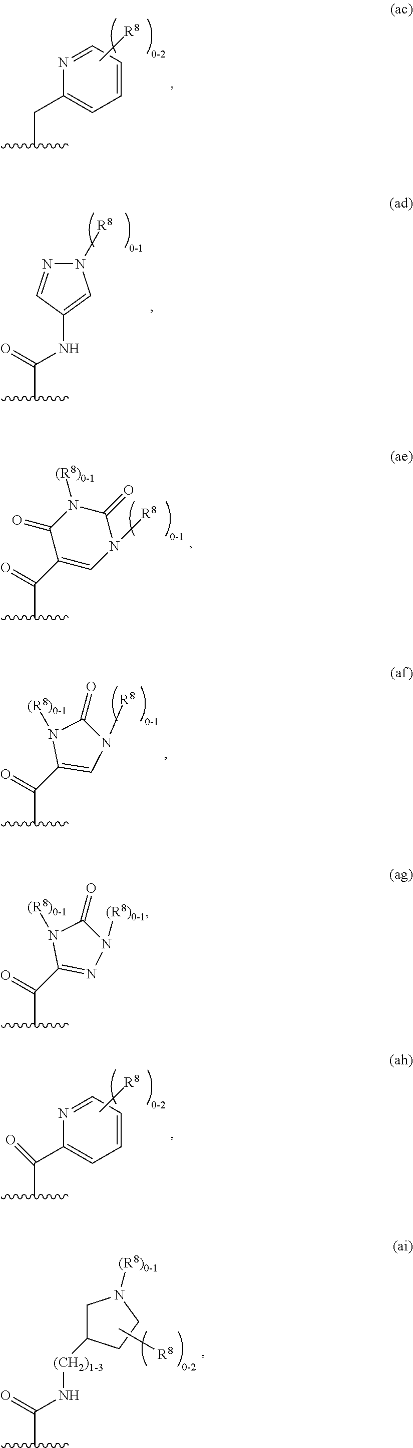

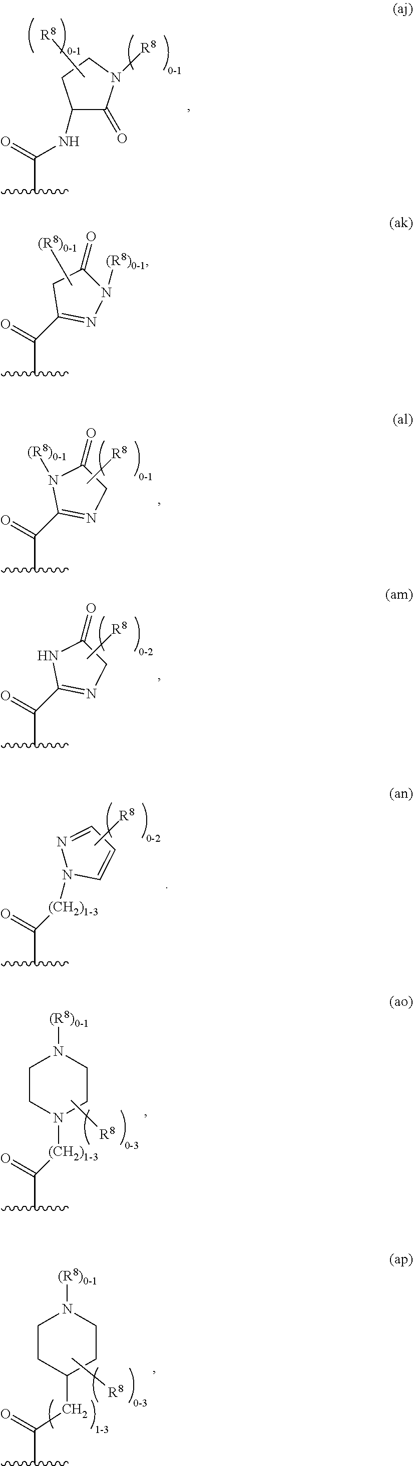

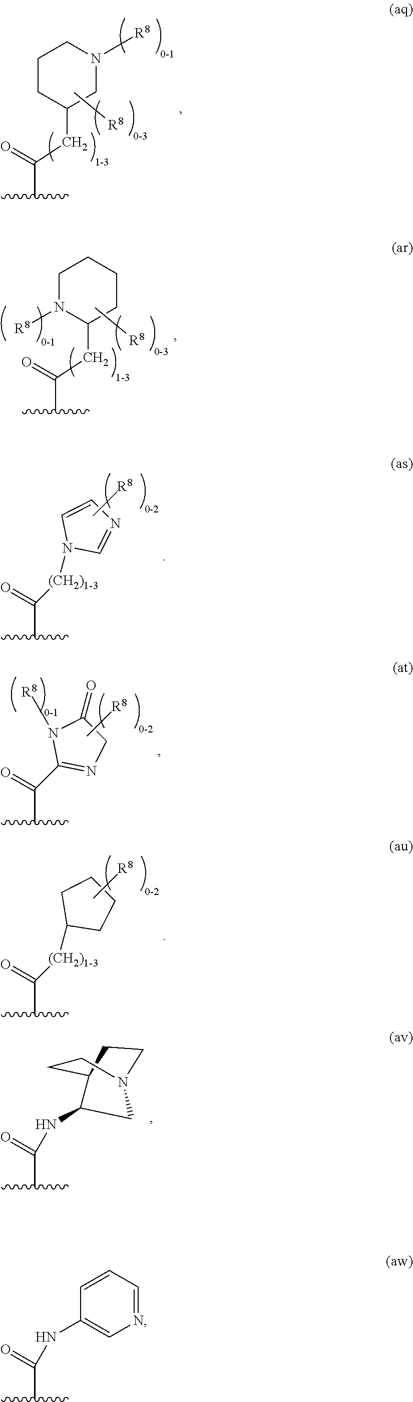

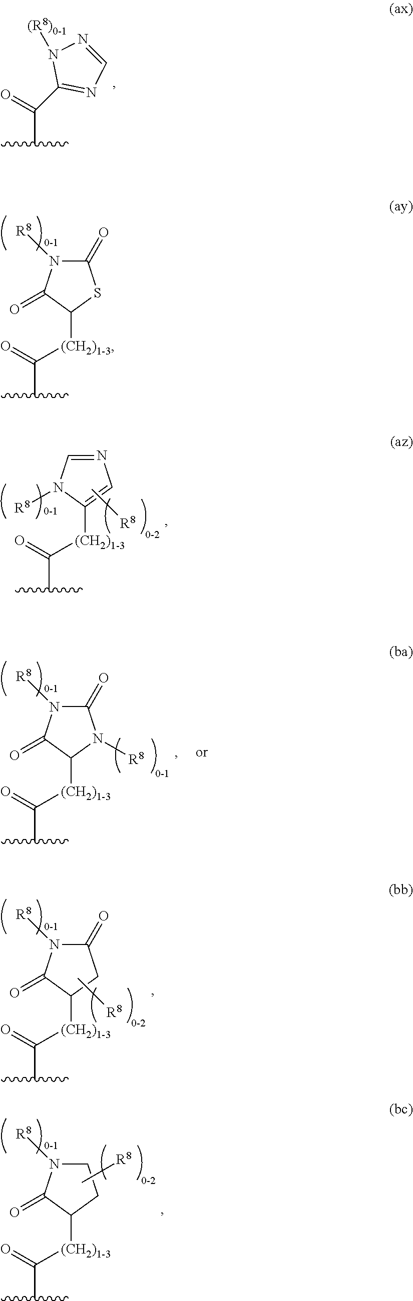

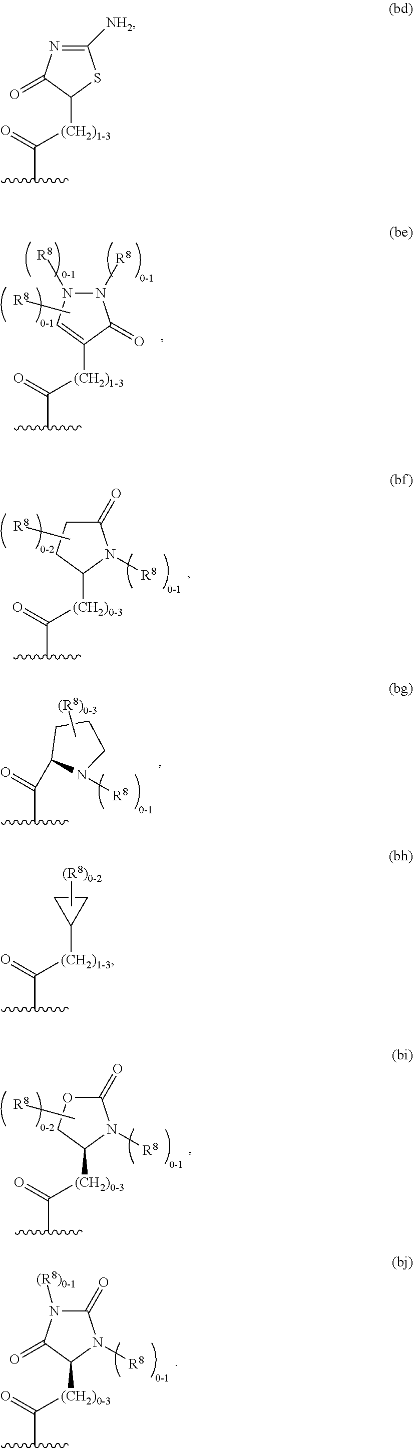

Embodiment 15(ii)(e) relates to Embodiment 15(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 15(iii) relates to Embodiment 15(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 15(iv) relates to Embodiment 15(ii) wherein R² is saturated 7-9 membered heterocycloalkyl comprising at least one nitrogen.

Embodiment 15(v) relates to Embodiment 15(ii) wherein R² is

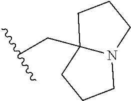 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 15(vi) relates to the compound of Embodiment 15(ii) wherein R³ is

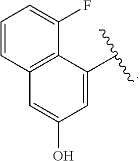 [SMILES: *c1cc(O)cc2cccc(F)c12]

Embodiment 15(vii) relates to Embodiment 15(vi) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 15(viii) relates to Embodiment 15(vi) wherein R² is saturated 7-9 membered heterocycloalkyl comprising at least one nitrogen.

Embodiment 15(ix) relates to Embodiment 15(vi) wherein R² is

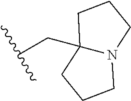 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 15(x) relates to the compound of Embodiment 15(ii) wherein R³ is

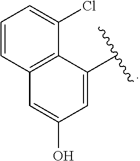 [SMILES: *c1cc(O)cc2cccc(Cl)c12]

Embodiment 15(xi) relates to Embodiment 15(x) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 15(xii) relates to Embodiment 15(x) wherein R² is saturated 7-9 membered heterocycloalkyl comprising at least one nitrogen.

Embodiment 15(xiii) relates to Embodiment 15(x) wherein R² is

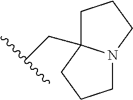 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 15(xiv) relates to the compound of Embodiment 15(ii) wherein R³ is

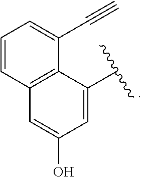 [SMILES: *c1cc(O)cc2cccc(C#C)c12]

Embodiment 15(xv) relates to Embodiment 15(xiv) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 15(xvi) relates to Embodiment 15(xiv) wherein R² is saturated 7-9 membered heterocycloalkyl comprising at least one nitrogen.

Embodiment 15(xvii) relates to Embodiment 15(xiv) wherein R² is

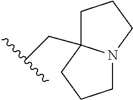 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 15(xviii) relates to the compound of Embodiment 15(ii) wherein R³ is

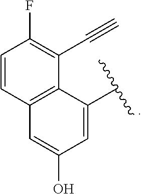 [SMILES: *c1cc(O)cc2ccc(F)c(C#C)c12]

Embodiment 15(xix) relates to Embodiment 15(xviii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 15(xx) relates to Embodiment 15(xviii) wherein R² is saturated 7-9 membered heterocycloalkyl comprising at least one nitrogen.

Embodiment 15(xxi) relates to Embodiment 15(xvii) wherein R² is

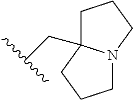 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 16(ii) of this disclosure relates to the compound according to any of Embodiments 1(ii), 2(ii), 3(ii), 4(ii), 5(ii), 6(ii), 7(ii), 8(ii), 9(ii), 10(ii), 11(ii), 12(ii), 13(ii), or 14(ii), wherein R³ is:

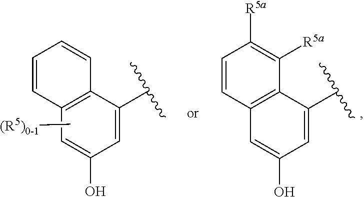

and R^{5a} is hydrogen, halo, or ethynyl.
Embodiment 16(ii)(a) relates to Embodiment 16(ii) wherein G is H.
Embodiment 16(ii)(b) relates to Embodiment 16(ii) wherein G is not H.
Embodiment 16(ii)(c) relates to Embodiment 16(ii) wherein G is not hydrogen, alkyl, hydroxyalkyl, alkenyl, benzyl, alkoxyalkyl, alkylaminoalkyl (e.g. -alkylene-N(R¹⁰)₂), alkylcarbonyl (e.g. —C(O)-Alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.
Embodiment 16(ii)(d) relates to Embodiment 16(ii) wherein G has one of the following Formulae:

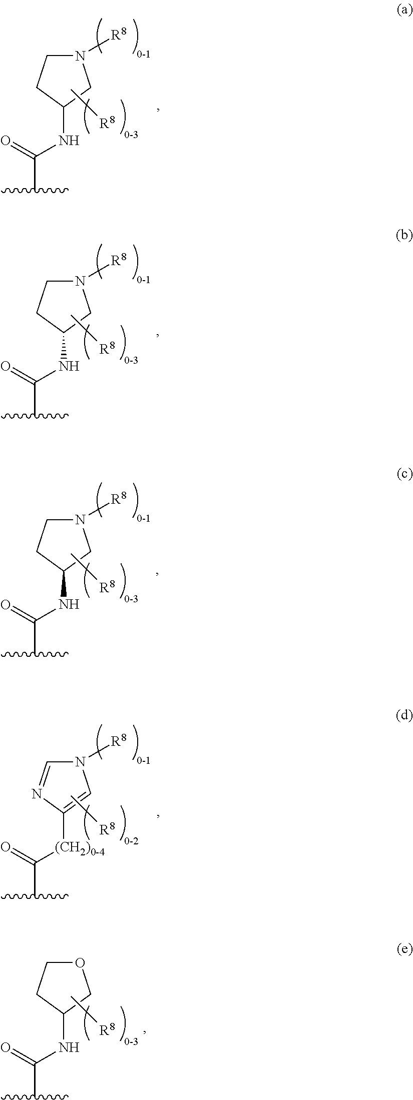

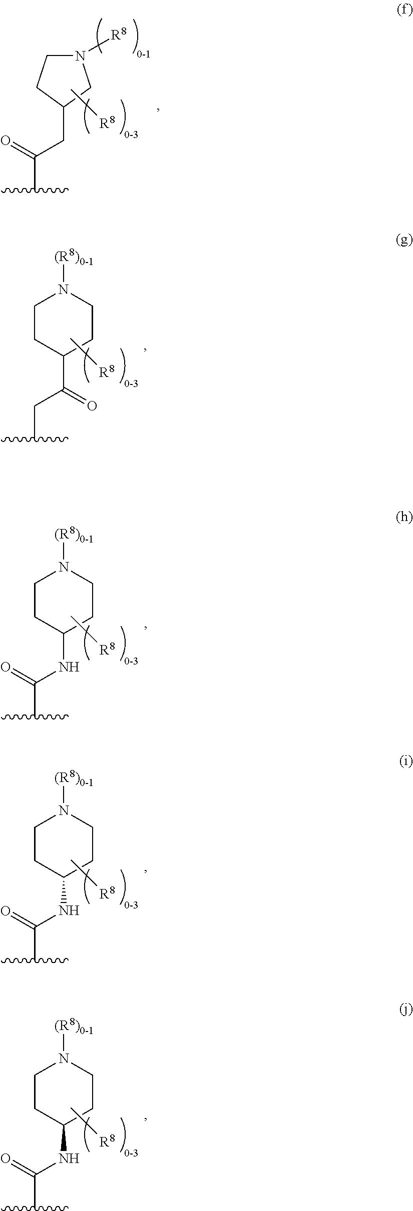

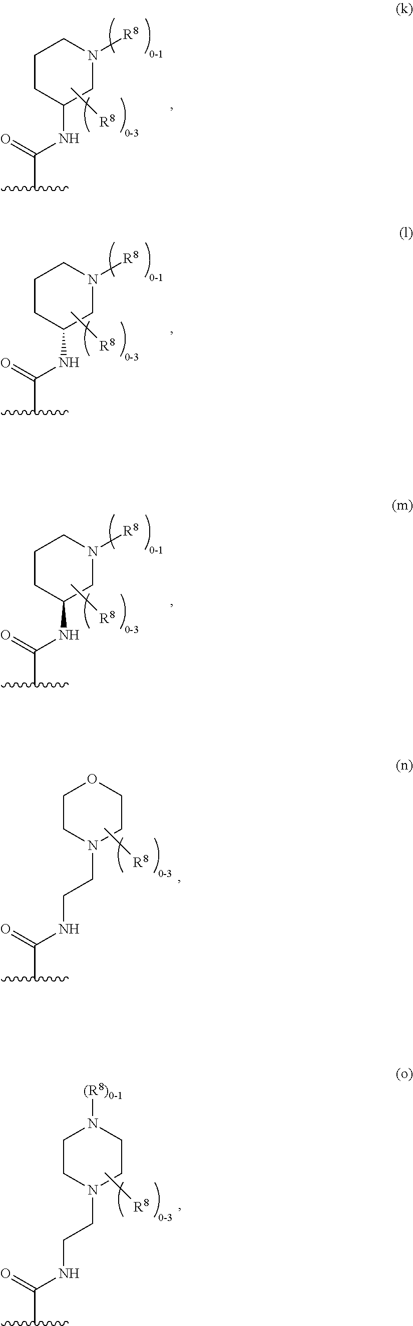

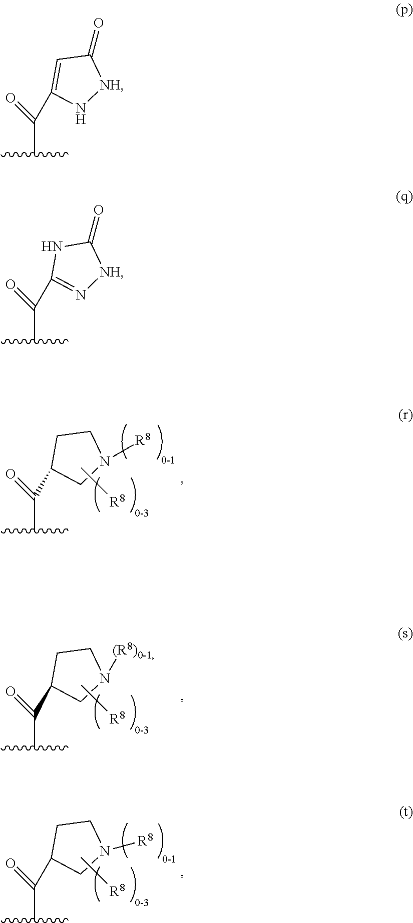

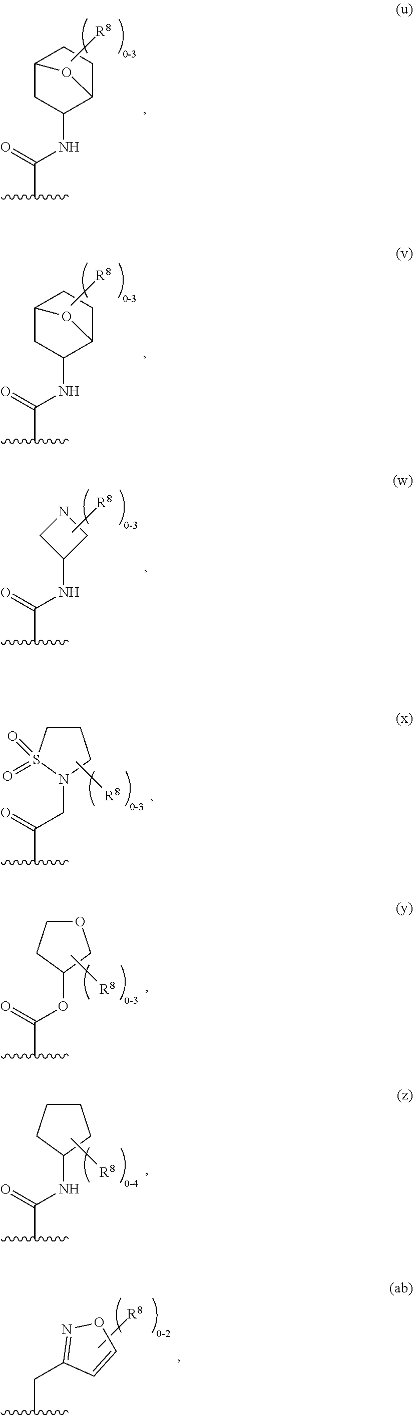

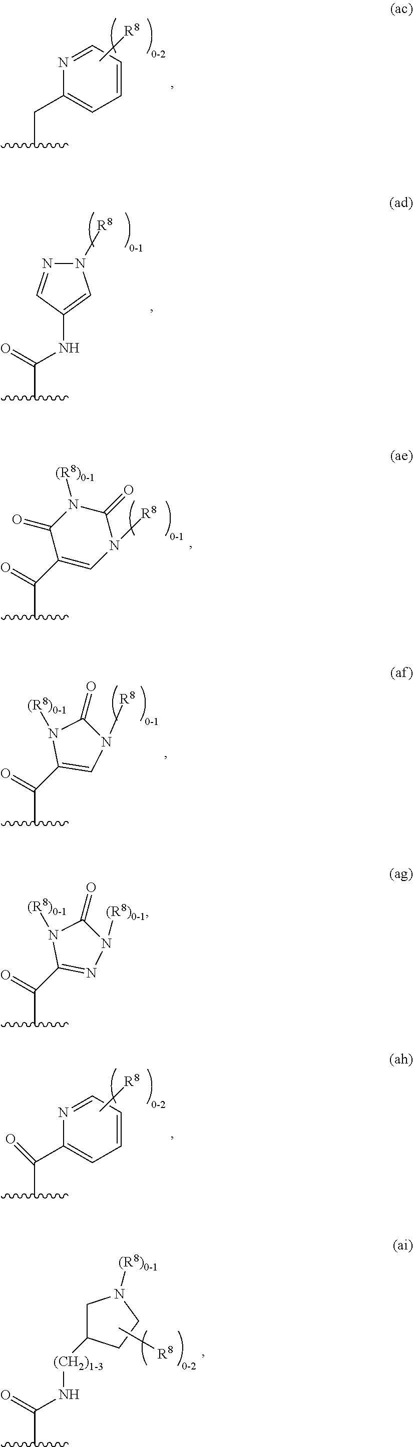

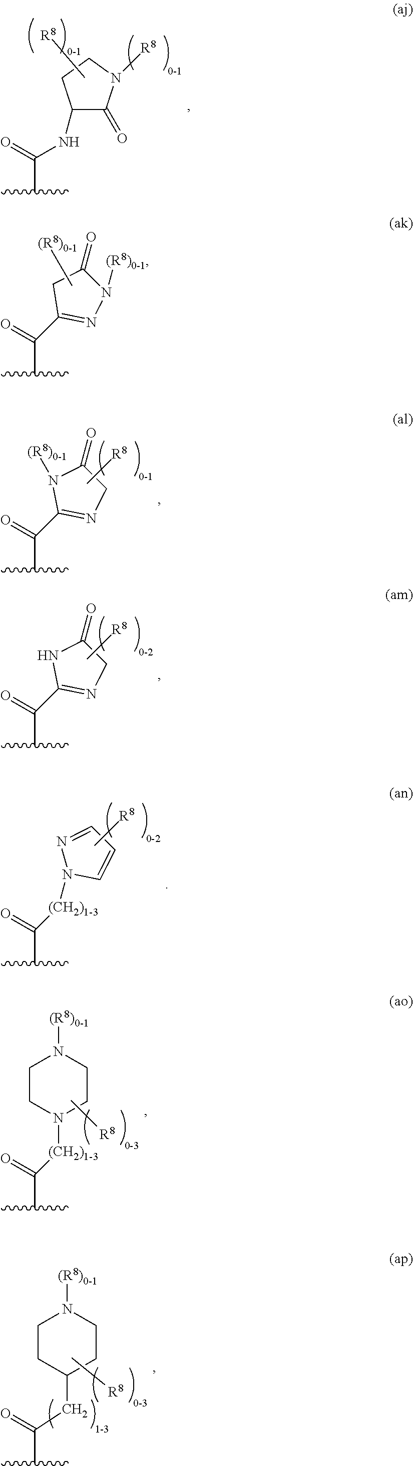

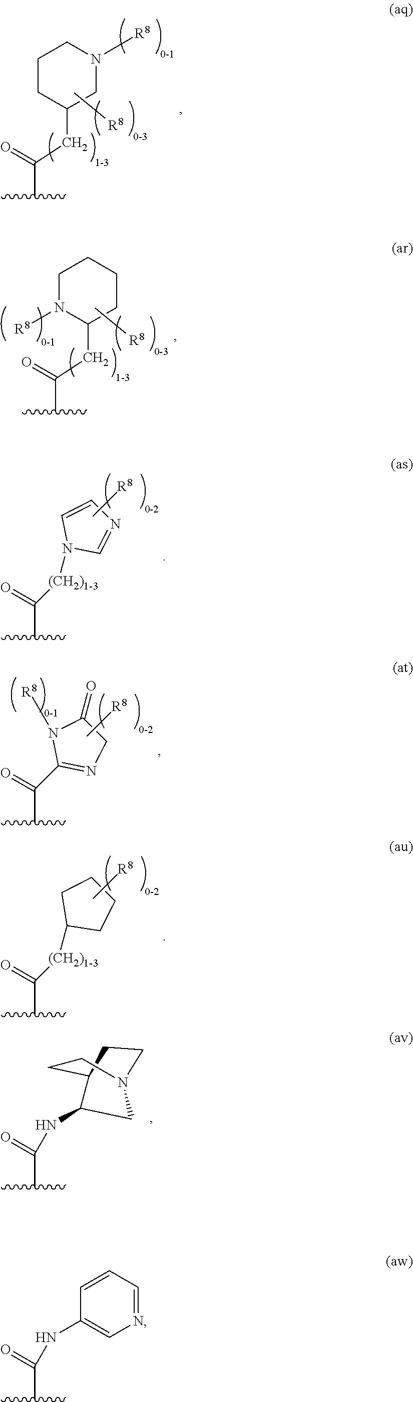

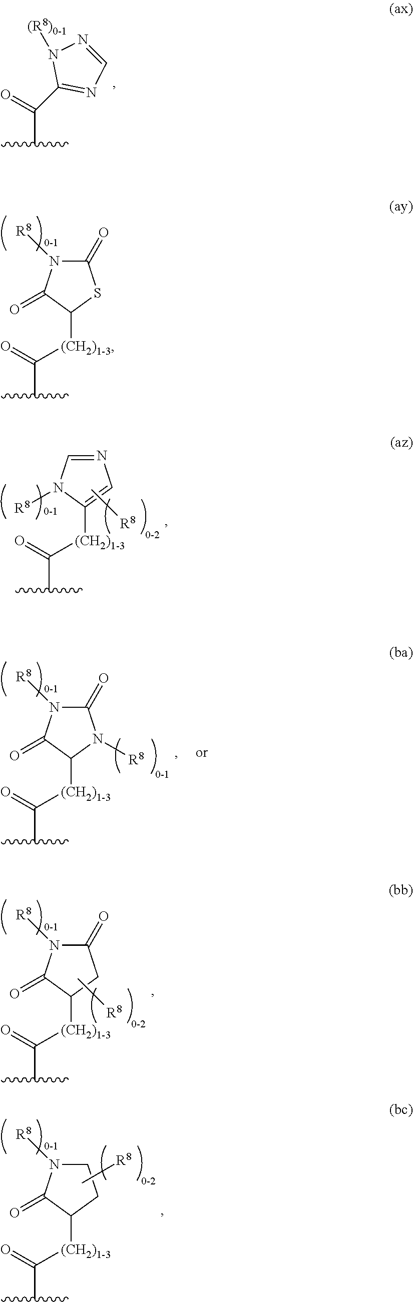

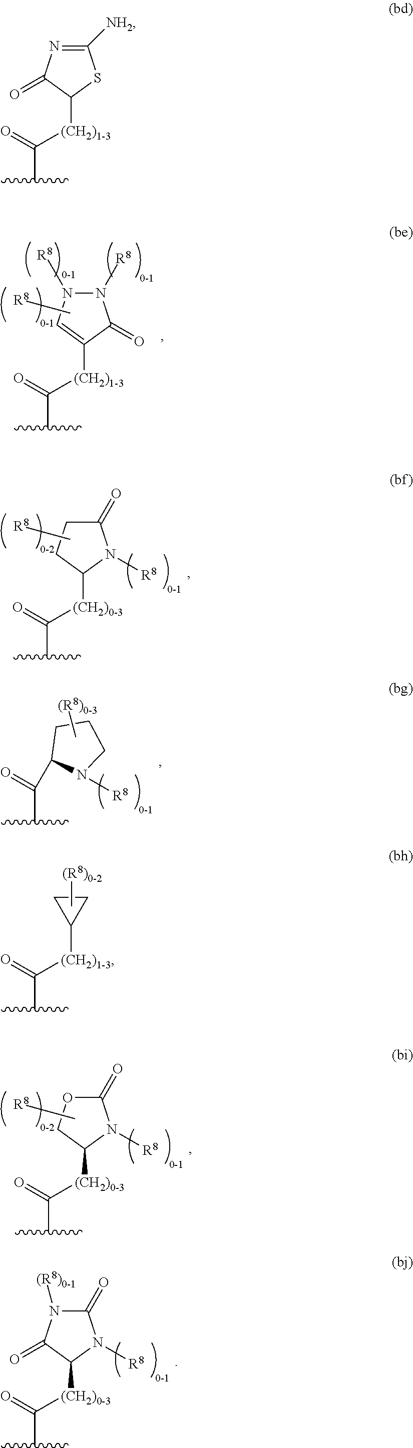

Embodiment 16(ii)(e) relates to Embodiment 16(ii) wherein G is cyanoalkylcarbonyl (—C(O)-alkyl-CN), -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

Embodiment 16(iii) relates to Embodiment 16(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 16(iv) relates to Embodiment 16(ii) wherein R² is saturated 7-9 membered heterocycloalkyl comprising at least one nitrogen.

Embodiment 16(v) relates to Embodiment 16(ii) wherein R² is

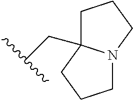 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 16(vi) relates to the compound of Embodiment 16(ii) wherein R³ is

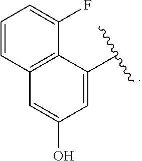 [SMILES: *c1cc(O)cc2cccc(F)c12]

Embodiment 16(vii) relates to Embodiment 16(vi) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 16(viii) relates to Embodiment 16(vi) wherein R² is saturated 7-9 membered heterocycloalkyl comprising at least one nitrogen.

Embodiment 16(ix) relates to Embodiment 16(vi) wherein R² is

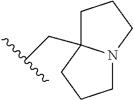 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 16(x) relates to the compound of Embodiment 16(ii) wherein R³ is

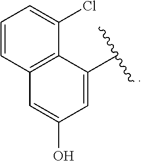 [SMILES: *c1cc(O)cc2cccc(Cl)c12]

Embodiment 16(xi) relates to Embodiment 16(x) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 16(xii) relates to Embodiment 16(x) wherein R² is saturated 7-9 membered heterocycloalkyl comprising at least one nitrogen.

Embodiment 16(xiii) relates to Embodiment 16(x) wherein R² is

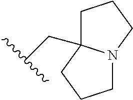 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 16(xiv) relates to the compound of Embodiment 16(ii) wherein R³ is

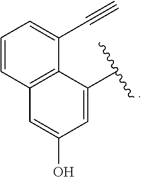 [SMILES: *c1cc(O)cc2cccc(C#C)c12]

Embodiment 16(xv) relates to Embodiment 16(xiv) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 16(xvi) relates to Embodiment 16(xiv) wherein R² is saturated 7-9 membered heterocycloalkyl comprising at least one nitrogen.

Embodiment 16(xvii) relates to Embodiment 16(xiv) wherein R² is

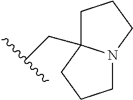 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 16(xviii) relates to the compound of Embodiment 16(ii) wherein R³ is

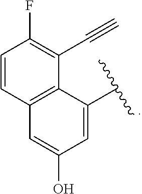 [SMILES: *c1cc(O)cc2ccc(F)c(C#C)c12]

Embodiment 16(xix) relates to Embodiment 16(xviii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 16(xx) relates to Embodiment 16(xviii) wherein R² is saturated 7-9 membered heterocycloalkyl comprising at least one nitrogen.

Embodiment 16(xxi) relates to Embodiment 16(xviii) wherein R² is

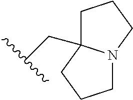 [SMILES: *CC12CCCN1CCC2]

that is substituted with 0-3 R⁴, provided that G is not alkyl, hydroxyalkyl, alkenyl, benzyl, alkoxyalkyl, alkylaminoalkyl (e.g. -alkylene-N(R¹⁰)₂), alkylcarbonyl (e.g. —C(O)-alkyl), alkylcarbonylaminoalkyl (e.g. -alkylene-N—C(O)—R¹⁰), cycloalkyl, heterocycloalkyl, bridged heterocycloalkyl, heterocyclic spiro ring, aryl, or heteroaryl.

Embodiment 17(ii) of this disclosure relates to the compound according to any of Embodiments 1(ii), 2(ii), 3(ii), 4(ii), 5(ii), 6(ii), 7(ii), 8(ii), 9(ii), 10(ii), 11(ii), 12(ii), 13(ii), 14(ii), 15(ii), or 16(ii), wherein:
L¹ is —C(O)N(H)—, —C(O)—, —C₁-C₃alkylene. —SO₂—, —SO₂N(H)—, or —C₁-C₃alkylene-C(O)—;
L² is absent or C₁-C₃alkylene; and
T² is one of the following formulae:

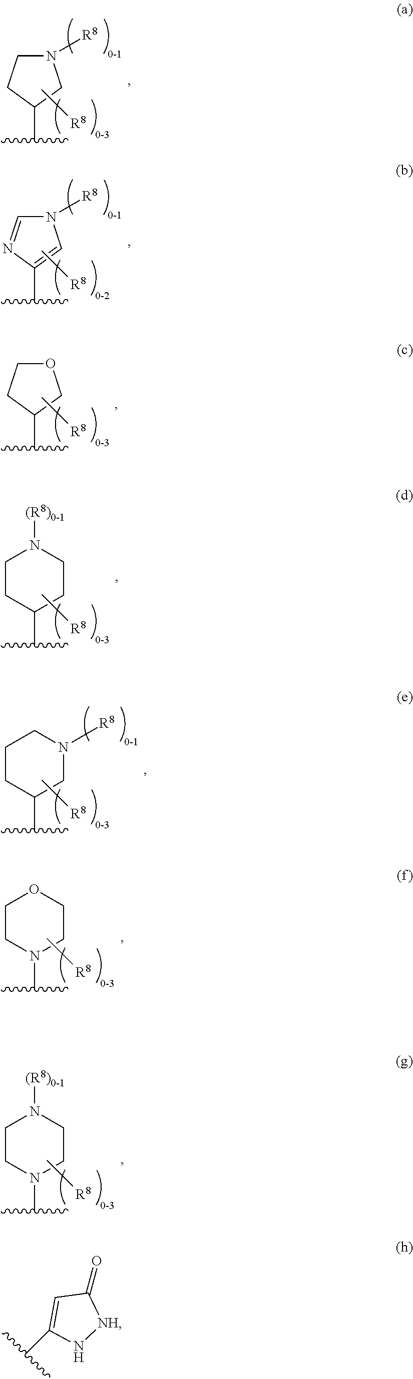

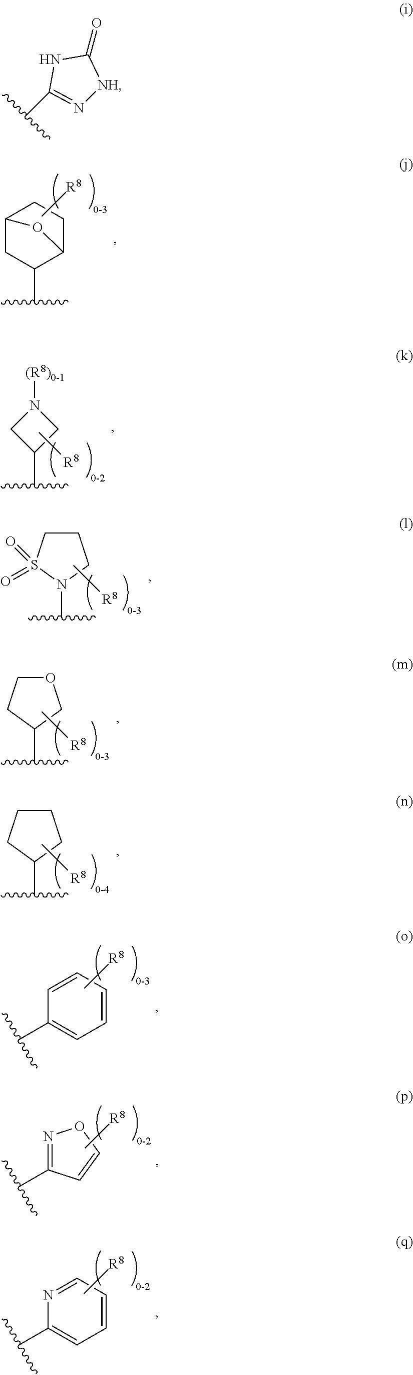

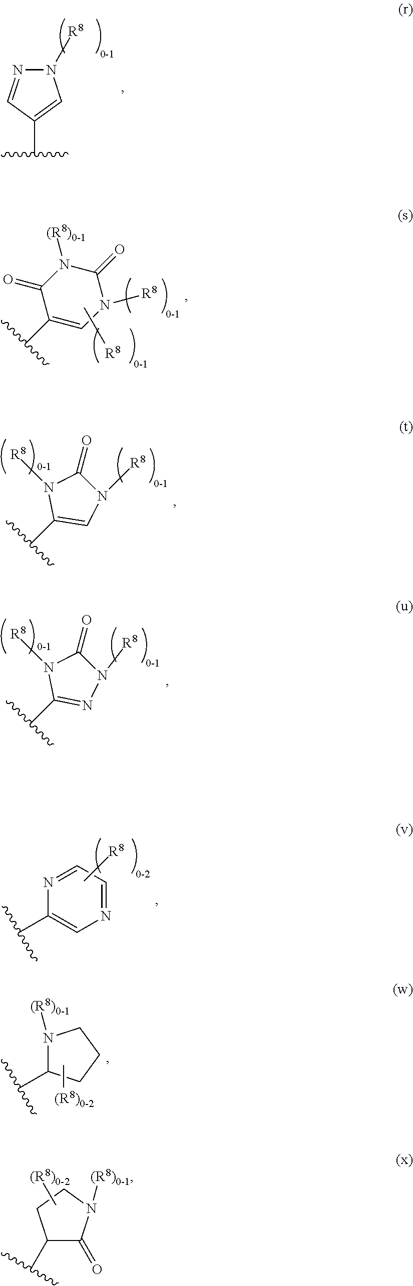

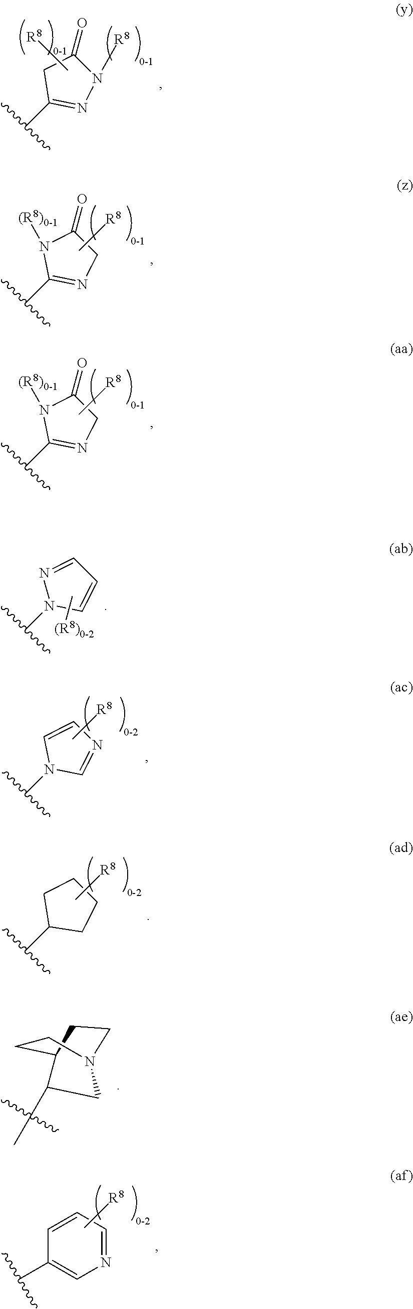

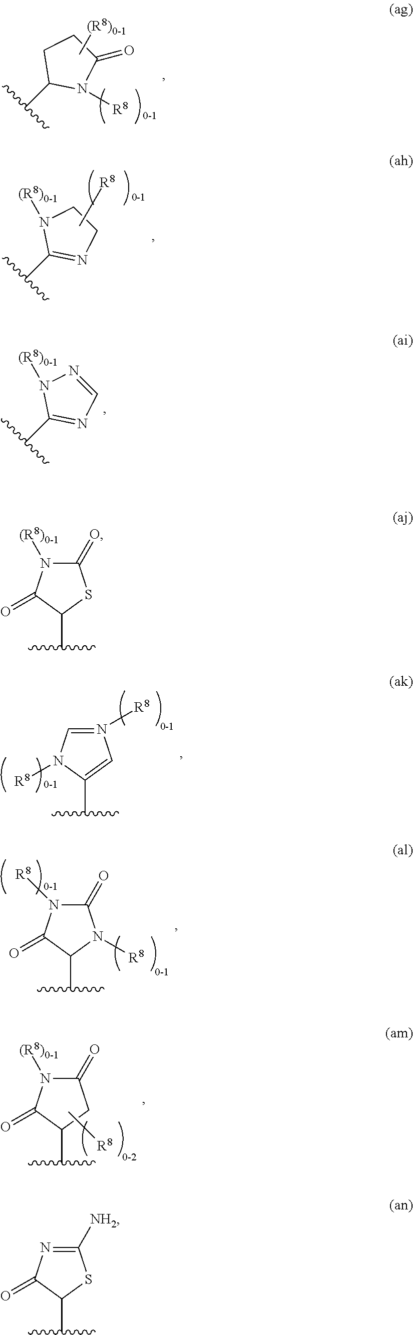

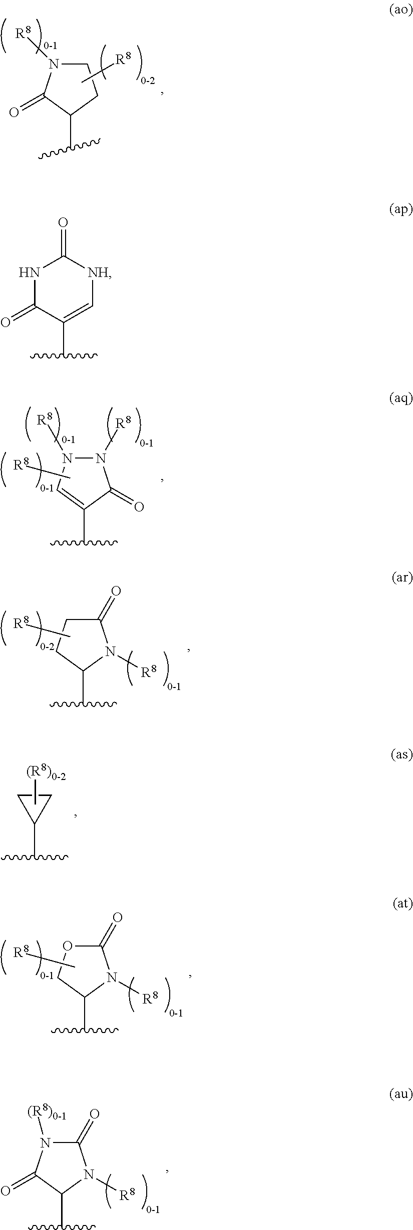

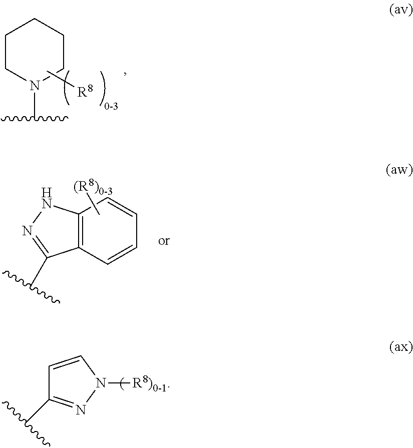

Embodiment 17(ii)(a) relates to Embodiment 17(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 17(ii)(b) relates to Embodiment 17(ii) wherein R² is saturated 7-9 membered heterocycloalkyl comprising at least one nitrogen.
Embodiment 17(ii)(c) relates to Embodiment 17(ii) wherein R² is

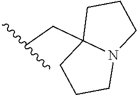 [SMILES: *CC12CCCN1CCC2]

that is substituted with 0-3 R⁴.

Embodiment 17(iii) of this disclosure relates to the compound according to Embodiments 17(ii) wherein:
L¹ is —C(O)N(H)—, —C(O)—, or C₁-C₃alkylene;
L² is absent or C₁-C₃alkylene; and
T² is one of the following formulae:

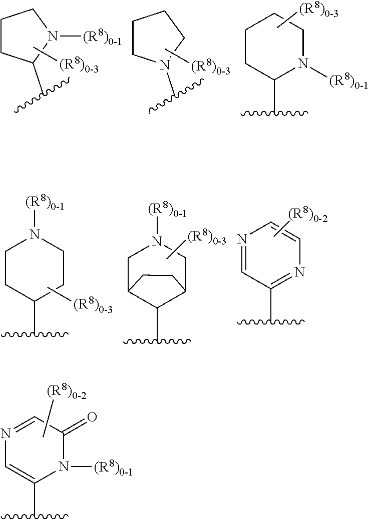

Embodiment 17(iii)(a) relates to Embodiment 17(iii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 17(iii)(b) relates to Embodiment 17(iii) wherein R² is saturated 7-9 membered heterocycloalkyl comprising at least one nitrogen.
Embodiment 17(iii)(c) relates to Embodiment 17(iii) wherein R² is

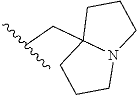 [SMILES: *CC12CCCN1CCC2]

that is substituted with 0-3 R⁴.

Embodiment 18(ii) of this disclosure relates to the compound according to any one of Embodiments 1(ii), 2(ii), 3(ii), 4(ii), 5(ii), 6(ii), 7(ii), 8(ii), 9(ii), 10(ii), 11(ii), 12(ii), 13(ii), 14(ii), 15(ii), 16(ii), or 17(h), wherein:
L¹ is —C(O)N(H)— or —C(O)—; and
L² is absent, —CH₂— or —CH₂—CH₂—.
Embodiment 18(ii)(a) relates to Embodiment 18(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 18(ii)(b) relates to Embodiment 18(ii) wherein R² is saturated 7-9 membered heterocycloalkyl comprising at least one nitrogen.
Embodiment 18(ii)(c) relates to Embodiment 18(ii) wherein R² is

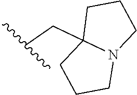 [SMILES: *CC12CCCN1CCC2]

that is substituted with 0-3 R⁴.

Embodiment 19(ii) of this disclosure relates to the compound according to any one of Embodiments 1(ii), 2(ii), 3(ii), 4(ii), 5(ii), 6(ii), 7(ii), 8(ii), 9(ii), 10(ii), 11(ii), 12(ii), 13(ii), 14(ii), 15(ii), or 16(ii) wherein:
L¹ is absent;
L² is absent; and
T² is H.
Embodiment 19(ii)(a) relates to Embodiment 19(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

Embodiment 19(ii)(b) relates to Embodiment 19(ii) wherein R² is saturated 7-9 membered heterocycloalkyl comprising at least one nitrogen.
Embodiment 19(ii)(c) relates to Embodiment 19(ii) wherein R² is

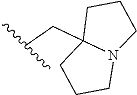 [SMILES: *CC12CCCN1CCC2]

that is substituted with 0-3 R⁴.

Embodiment 20(ii) of this disclosure relates to the compound according to any one of 1(ii), 2(ii), 3(ii), 4(ii), 5(ii), 6(ii), 7(ii), 8(ii), 9(ii), 10(ii), 11(ii), 12(ii), 13(ii), 14(ii), 15(ii), or 16(ii), wherein G is:
—CH₂—C(H)(OH)—CH₂—OH, —C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, —C(O)(CH₂)₂C(O)OCH₃, or one of the following Formulae:

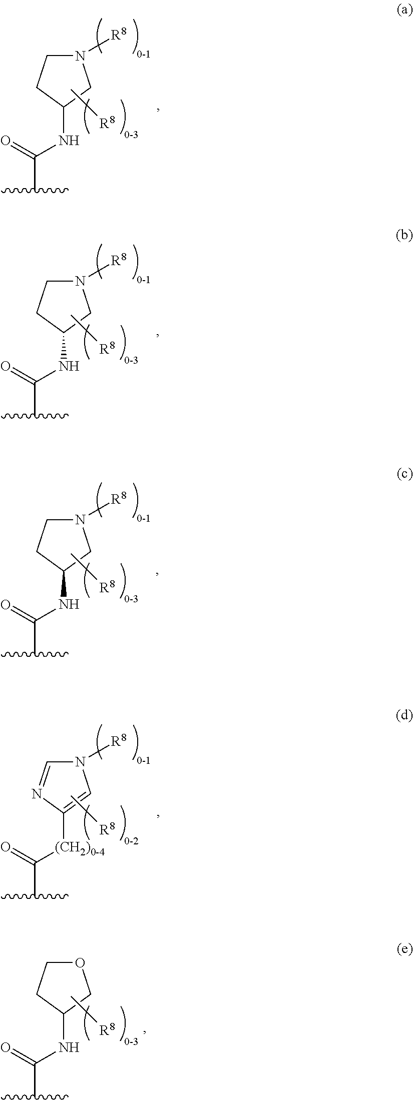

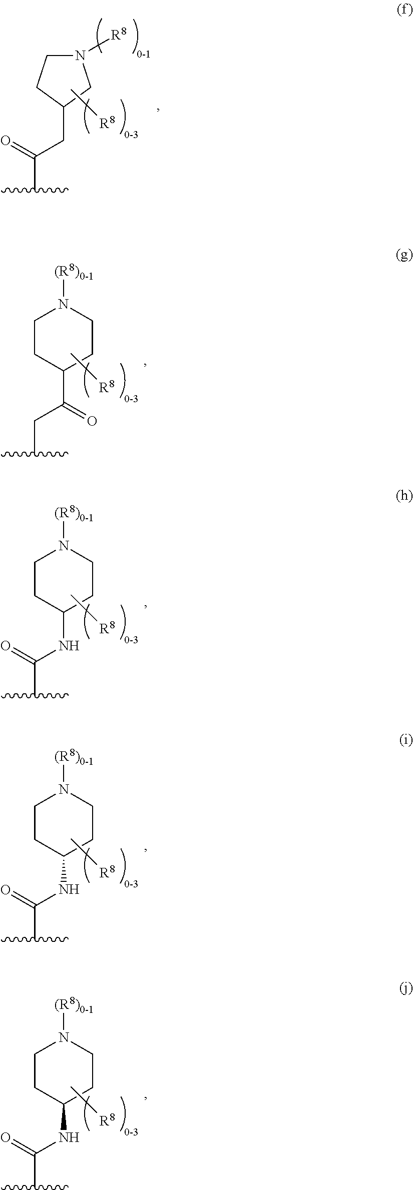

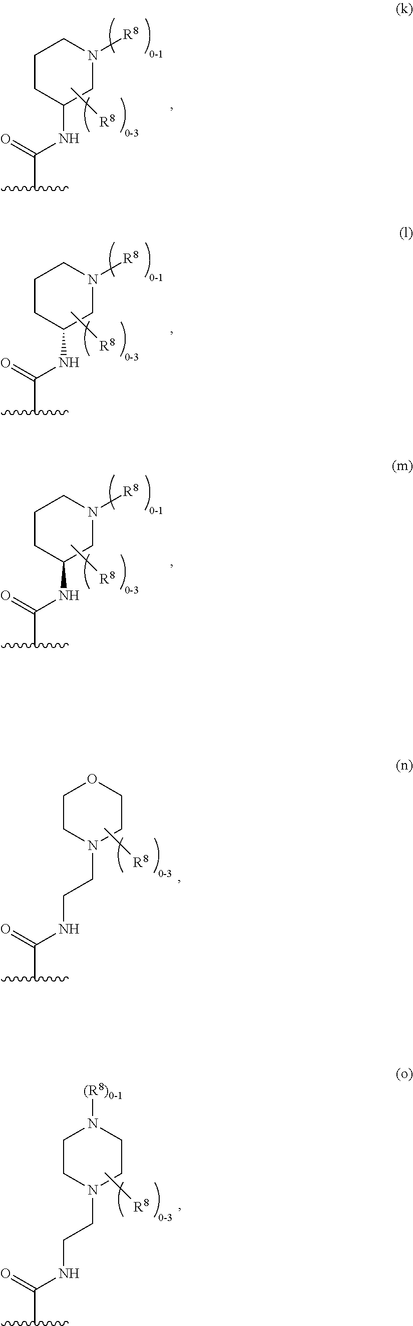

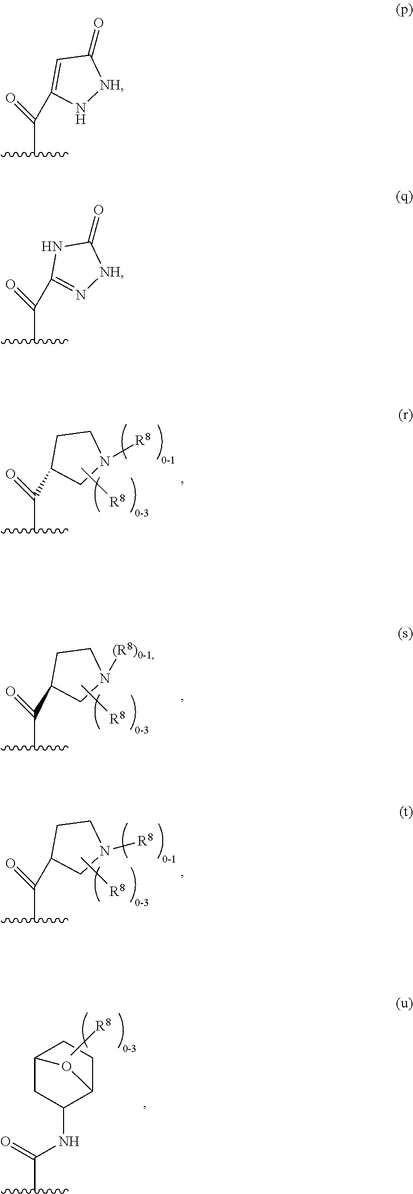

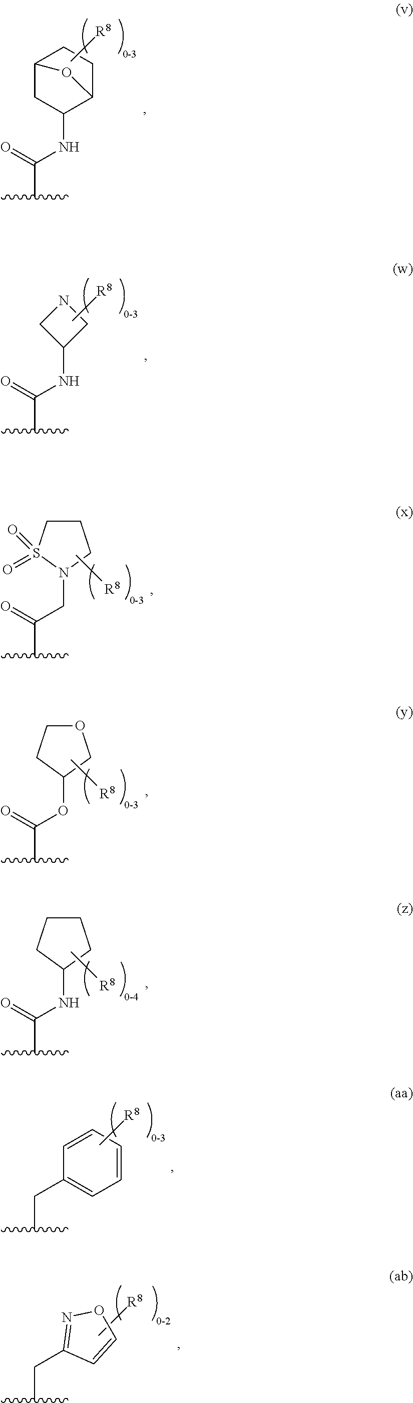

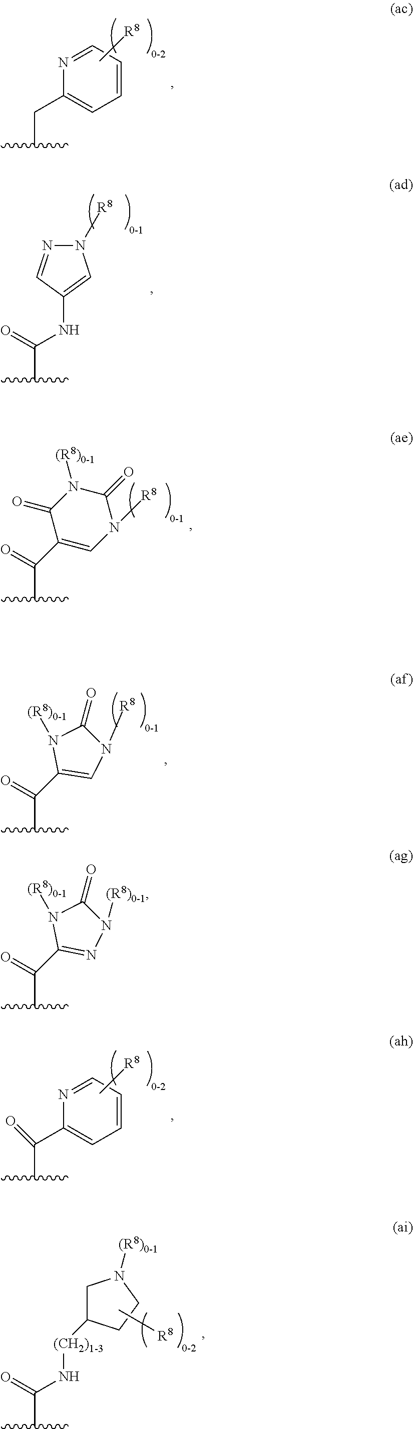

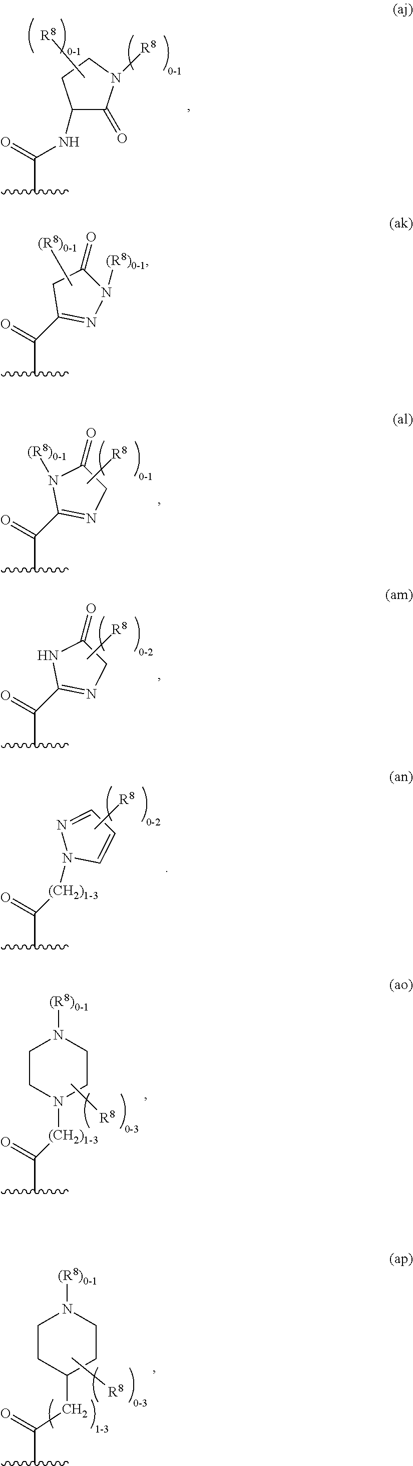

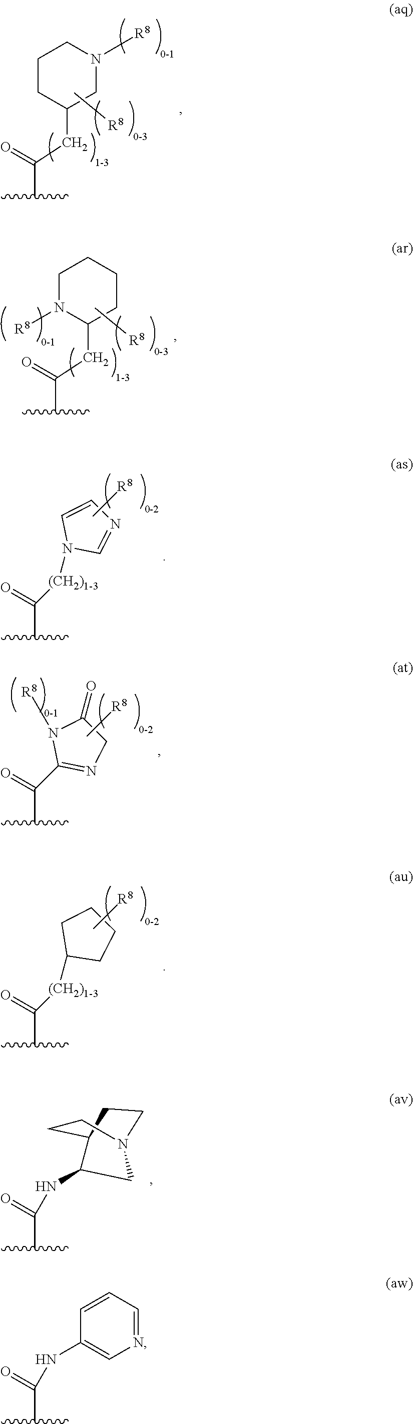

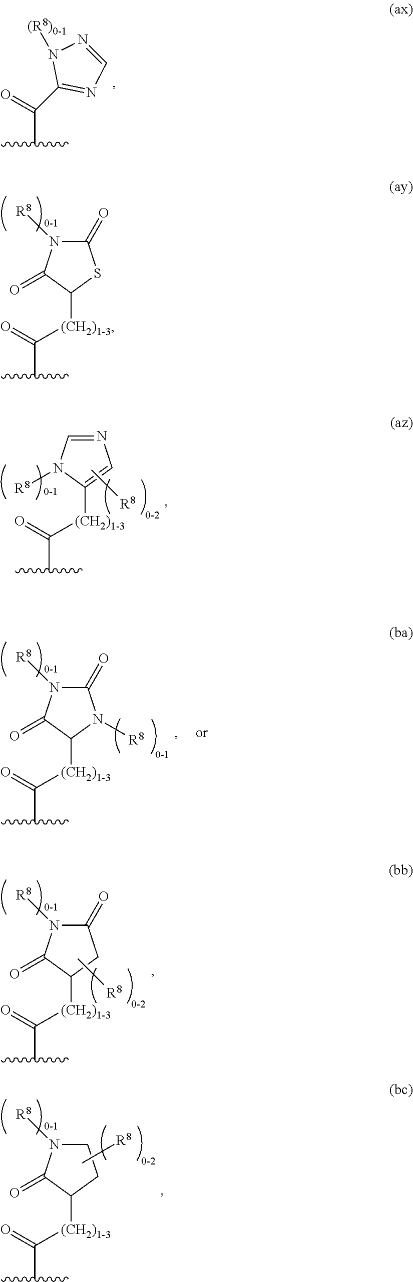

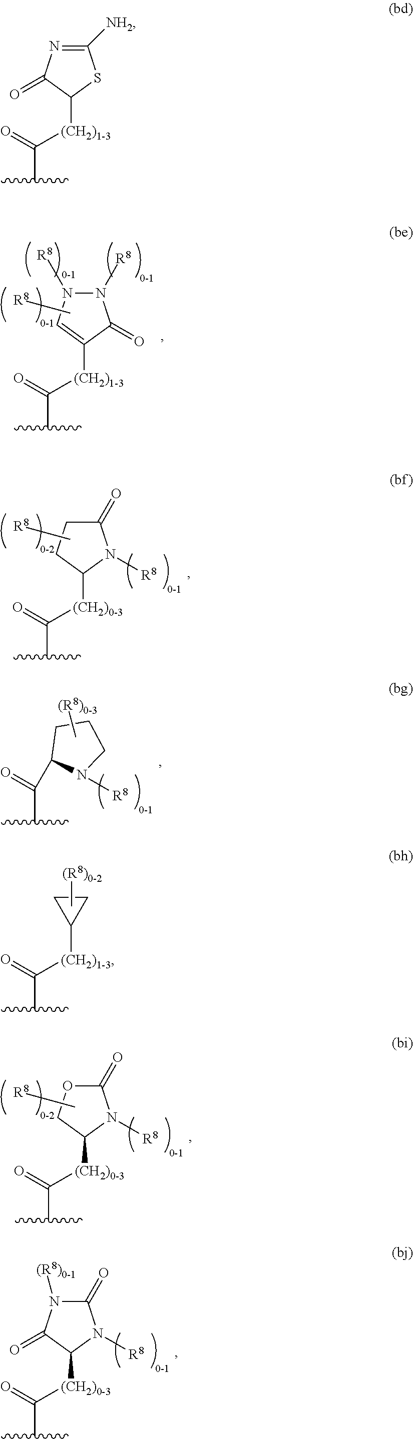

Embodiment 20(ii)(a) relates to Embodiment 20(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 20(ii)(b) relates to Embodiment 20(ii) wherein R² is saturated 7-9 membered heterocycloalkyl comprising at least one nitrogen.
Embodiment 20(ii)(c) relates to Embodiment 20(ii) wherein R² is

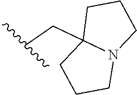 [SMILES: *CC12CCCN1CCC2]

that is substituted with 0-3 R⁴.

Embodiment 21 (ii) of this disclosure relates to the compound according to Embodiment 20(ii), wherein G is —CH₂—C(H)(OH)—CH₂—OH, —C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, or —C(O)(CH₂)₂C(O)OCH₃.
Embodiment 21(ii)(a) relates to Embodiment 21 (ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 21 (ii)(b) relates to Embodiment 21 (ii) wherein R² is saturated 7-9 membered heterocycloalkyl comprising at least one nitrogen.
Embodiment 21 (ii)(c) relates to Embodiment 21 (ii) wherein R² is

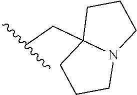 [SMILES: *CC12CCCN1CCC2]

that is substituted with 0-3 R⁴.

Embodiment 22(ii) of this disclosure relates to the compound according to Embodiment 20(ii), wherein G is one of formulae (a), (b), (c), (d), (e), (h), (i), (j), (k), (l), (m), (p), (q), (r), (s), (t), (u), (v), (w), (z), (ad), (ae), (af), (ag), (ah), (aj), (ak), (al), (am), (at), (av), (aw), (ax), or (bg).
Embodiment 22(ii)(a) relates to Embodiment 22(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 22(ii)(b) relates to Embodiment 22(ii) wherein R² is saturated 7-9 membered heterocycloalkyl comprising at least one nitrogen.
Embodiment 22(ii)(c) relates to Embodiment 22(ii) wherein R² is

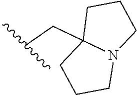 [SMILES: *CC12CCCN1CCC2]

that is substituted with 0-3 R⁴.

Embodiment 23(ii) of this disclosure relates to the compound according to Embodiment 20(ii), wherein G is one of formulae (d), (f), (n), (o), (x), (ai), (an), (ao), (ap), (aq), (ar), (as), (au), (ay), (az), (ba), (bb), (be), (bd), (be), (bf), (bi), or (bj).
Embodiment 23(ii)(a) relates to Embodiment 23(ii) wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.
Embodiment 23(ii)(b) relates to Embodiment 23(ii) wherein R² is saturated 7-9 membered heterocycloalkyl comprising at least one nitrogen.
Embodiment 23(ii)(c) relates to Embodiment 23(ii) wherein R² is

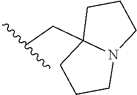 [SMILES: *CC12CCCN1CCC2]

that is substituted with 0-3 R⁴.

Embodiment 24(ii) of this disclosure relates to the compound according to any one of Embodiments 21 (ii), 22(ii), or 23(ii), wherein R² is

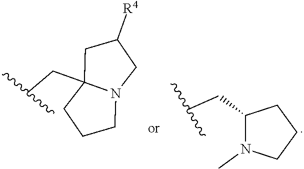

### and R⁴ is H, Cl, or F.

Embodiment 24(iii) relates to the compound of Embodiment 24(ii) wherein R⁴ is H and R² has the following formula:

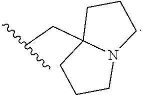 [SMILES: *CC12CCCN1CCC2]

Embodiment 24(iv) relates to the compound of Embodiment 24(ii) wherein R⁴ is F and R² has the following formula:

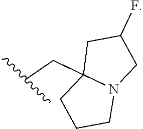 [SMILES: *CC12CCCN1CC(F)C2]

Embodiment 24(v) relates to the compound of Embodiment 24(ii) wherein R⁴ is Cl and R² has the following formula:

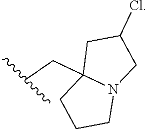 [SMILES: *CC12CCCN1CC(Cl)C2]

Embodiment 25(ii) of this disclosure relates to the compound according to Embodiment 1(ii) selected from Table IA, or a pharmaceutically acceptable salt thereof.
Embodiment 25(ii)(a) relates to the compound of Embodiment 25(ii) wherein G is H.
Embodiment 25(ii)(b) relates to the compound of Embodiment 25(ii) wherein G is not H.

Embodiment 25(iii) of this disclosure relates to the compound according to Embodiment 1(ii) selected from Table IB, or a pharmaceutically acceptable salt thereof.
Embodiment 25(iii)(a) relates to the compound of Embodiment 25(iii) wherein G is H.
Embodiment 25(iii)(b) relates to the compound of Embodiment 25(iii) wherein G is not H.
Embodiment 25(iv) relates to Embodiment 25(ii) wherein R² is

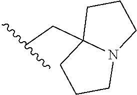 [SMILES: *CC12CCCN1CCC2]

that is substituted with 0-3 R⁴.

Additionally, the formulae described in this disclosure are intended to include hydrated or solvated as well as unhydrated or unsolvated forms of the identified structures. For example, the indicated compounds include both hydrated and non-hydrated forms. Other examples of solvates include the structures in combination with a suitable solvent, such as isopropanol, ethanol, methanol, dimethyl sulfoxide, ethyl acetate, acetic acid, or ethanolamine.

Additionally, the formulae described in this disclosure are intended to include hydrated or solvated as well as unhydrated or unsolvated forms of the identified structures. For example, the indicated compounds include both hydrated and non-hydrated forms. Other examples of solvates include the structures in combination with a suitable solvent, such as isopropanol, ethanol, methanol, dimethyl sulfoxide, ethyl acetate, acetic acid, or ethanolamine.

### III. Formulations and Administration

Embodiment 26 of this disclosure relates to a pharmaceutical composition comprising a compound in any one of Embodiments 1-25, including any subembodiments thereof (such as those indicated with designations “(a)”, “(b)”, “(h)”, “(hi)”, “(iv)”, “(v)”, or “(vi)”, “(vii)”, “(viii)”, “(ix)”, “(x)”, etc.), and a pharmaceutically acceptable carrier.

Embodiment 27 of this disclosure relates to a pharmaceutical composition of Embodiment 26, further comprising a second pharmaceutical agent.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in The Science and Practice of Pharmacy, 21^{st} edition, Lippincott, Williams and Wilkins, Philadelphia, Pa., 2005 (hereby incorporated by reference herein).

Compounds of the present disclosure (i.e. any of the compounds described in Embodiments 1-25, including any of the subembodiments thereof) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, the compounds can be administered by oral administration. For oral administration, for example, the compounds can be formulated into conventional oral dosage forms such as capsules, tablets, and liquid preparations such as syrups, elixirs, and concentrated drops.

For inhalants, compounds of the disclosure may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the disclosure may also be used in combination with other inhaled therapies, for example corticosteroids such as fluticasone propionate, beclomethasone dipropionate, triamcinolone acetonide, budesonide, and mometasone furoate; beta agonists such as albuterol, salmeterol, and formoterol; anticholinergic agents such as ipratropium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers.

In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the disclosure are formulated in sterile liquid solutions, such as in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

The amounts of various compounds to be administered can be determined by standard procedures taking into account factors such as the compound IC₅₀, the biological half-life of the compound, the age, size, and weight of the subject, and the indication being treated. The importance of these and other factors are well known to those of ordinary skill in the art. Generally, a dose will be between about 0.01 and 50 mg/kg, or 0.1 and 20 mg/kg of the subject being treated. Multiple doses may be used.

The compounds of the disclosure may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the disclosure or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present disclosure, or at the same time as a compound of the disclosure. Use in combination also includes use with a therapy or medical procedure that is administered once or infrequently, such as surgery, along with a compound of the disclosure administered within a short time or longer time before or after the other therapy or procedure. In some embodiments, the present disclosure provides for delivery of compounds of the disclosure and one or more other drug therapeutics delivered by a different route of administration or by the same route of administration. The use in combination for any route of administration includes delivery of compounds of the disclosure and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the disclosure. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the disclosure and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### IV. Methods of Use

The methods and compounds will typically be used in therapy for human subjects. However, they may also be used to treat similar or identical indications in other animal subjects.

In certain embodiments, the patient is 60 years or older and relapsed after a first line cancer therapy. In certain embodiments, the patient is 18 years or older and is relapsed or refractory after a second line cancer therapy. In certain embodiments, the patient is 60 years or older and is primary refractory to a first line cancer therapy. In certain embodiments, the patient is 70 years or older and is previously untreated. In certain embodiments, the patient is 70 years or older and is ineligible and/or unlikely to benefit from cancer therapy.

In certain embodiments, the therapeutically effective amount used in the methods provided herein is at least 10 mg per day. In certain embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500 mg per day. In other embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, 5000 mg per day or more. In certain embodiments, the compound is administered continuously.

In certain embodiments, provided herein is a method for treating a diseases or condition mediated by KRAS by administering to a mammal having a disease or condition at least 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, 5000 mg per day of any of the compounds described in a compound in one of Embodiments 1-36, or a pharmaceutically acceptable salt, deuterated analog, a tautomer or a stereoisomer thereof, and wherein the compound is administered on an empty stomach.

Embodiment 28 or this disclosure relates to a method for treating a subject with a disease or condition mediated by KRAS, said method comprising administering to the subject an effective amount of a compound in one of Embodiments 1-25 (including any subembodiments thereof), or a pharmaceutically acceptable salt, a solvate, a tautomer, a stereoisomer, or a deuterated analog thereof, or a pharmaceutical composition in one of Embodiments 26-27.

Embodiment 29 or this disclosure relates the method for treatment of a disease or condition according to Embodiment 28, wherein the disease or condition is a neoplastic disorder, a cancer, an age-related disease, an inflammatory disorder, a cognitive disorder or a neurodegenerative disease.

Embodiment 30 or this disclosure relates the method of Embodiment 29, wherein the disease or condition is pancreatic cancer (e.g., pancreatic carcinoma, pancreatic ductal adenocarcinoma, pancreatic adenocarcinoma, pancreatic adenosquamous carcinoma, pancreatic neuroendocrine neoplasm), colorectal cancer, (e.g., colorectal carcinoma, colorectal adenocarcinoma, colorectal mucinous adenocarcinoma, colorectal neuroendocrine carcinoma, malignant colorectal neoplasm) malignant solid tumor, rectal carcinoma (e.g., rectal adenocarcinoma), endometrial endometriod carcinoma, appendix carcinoma, mucinous adenocarcinoma, cholangiocarcinoma, extrahepatic cholangiocarcinoma, bladder cancer, bladder urothelial carcinoma, lung cancer (e.g., lung adenocarcinoma, non-small cell lung cancer, small cell lung carcinoma, squamous cell lung carcinoma), ampulla of vater carcinoma, ovarian cancer (e.g., ovarian mucinous adenocarcinoma, ovarian serous adenocarcinoma, low grade ovarian serous adenocarcinoma), gastric adenocarcinoma, malignant small intestinal neoplasm, poorly differentiated adenocarcinoma, acute myeloid leukemia, breast cancer, breast invasive ductal carcinoma, ampulla vater pancreatobiliary type adenocarcinoma, glioma, multiple myeloma, myelodysplastic syndrome, or small intestine carcinoma.

Embodiment 30(a) or this disclosure relates the method of Embodiment 30, wherein the disease or condition is pancreatic cancer, colorectal cancer, malignant solid tumor, rectal carcinoma, lung cancer, acute myeloid leukemia, glioma, multiple myeloma, or myelodysplastic syndrome.

Embodiment 30(b) or this disclosure relates the method of Embodiment 30(a), wherein the disease or condition is pancreatic carcinoma, pancreatic ductal adenocarcinoma, pancreatic adenocarcinoma, colorectal carcinoma, colorectal adenocarcinoma, malignant colorectal neoplasm, malignant solid tumor, rectal adenocarcinoma, lung adenocarcinoma, non-small cell lung cancer, small cell lung carcinoma, squamous cell lung carcinoma, acute myeloid leukemia, glioma, multiple myeloma, or myelodysplastic syndrome.

Embodiment 30(c) or this disclosure relates the method of Embodiment 29, wherein the disease or condition is mediated by KRAS G12D.

Embodiment 30(d) or this disclosure relates the method of Embodiment 30(c), wherein the disease or condition is pancreatic ductal adenocarcinoma colorectal carcinoma, pancreatic carcinoma, pancreatic adenocarcinoma, malignant colorectal neoplasm patients, malignant colorectal neoplasm, rectal carcinoma, lung adenocarcinoma, non-small cell lung carcinoma, colorectal carcinoma, malignant solid tumor, acute myeloid leukemia, squamous cell lung carcinoma, small cell lung carcinoma, glioma, myelodysplastic syndrome, breast cancer, gastric carcinoma, ovarian carcinoma, multiple myeloma patients, hepatocellular carcinoma, head and neck squamous cell carcinoma, glioblastoma or thyroid gland undifferentiated carcinoma. Embodiment 30(e) or this disclosure relates the method of Embodiment 29, wherein the disease or condition is mediated by KRAS G12V.
Embodiment 30(f) or this disclosure relates the method of Embodiment 30(e), wherein the disease or condition is pancreatic ductal adenocarcinoma, pancreatic ductal adenocarcinoma, colorectal adenocarcinoma patients, colorectal carcinoma, rectal adenocarcinoma, pancreatic carcinoma, pancreatic adenocarcinoma, malignant colorectal neoplasm, lung adenocarcinoma, non-small cell lung carcinoma, colorectal carcinoma, malignant solid tumor, acute myeloid leukemia, squamous cell lung carcinoma, small cell lung carcinoma, glioma, myelodysplastic syndrome or multiple myeloma.

### V. Combination Therapy

KRAS modulators may be usefully combined with another pharmacologically active compound, or with two or more other pharmacologically active compounds, particularly in the treatment of cancer. In one embodiment, the composition includes any one or more compound(s) as described herein along with one or more compounds that are therapeutically effective for the same disease indication, wherein the compounds have a synergistic effect on the disease indication. In one embodiment, the composition includes any one or more compound(s) as described herein effective in treating a cancer and one or more other compounds that are effective in treating the same cancer, further wherein the compounds are synergistically effective in treating the cancer.

Embodiment 31 or this disclosure relates the method according to any one of Embodiments 28-30, further comprising administering one or more additional therapeutic agents.

Embodiment 32 or this disclosure relates the method according to Embodiment 31, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from the group consisting of azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immunotherapy agent selected from a PD-1 or PD-L1 inhibitor; v) a hormone or hormone antagonist selected from the group consisting of enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38 (7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, AEE-788, AG-013736, AMG 706, AMN107, BMS-354825, BMS-599626, UCN-01 (7-hydroxystaurosporine), vemurafenib, dabrafenib, trametinib, cobimetinib selumetinib and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; and xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, a mTOR inhibitor, a PI3K inhibitor, a Cdk4 inhibitor, an Akt inhibitor, a Hsp90 inhibitor, a famesyltransferase inhibitor or an aromatase inhibitor (anastrozole letrozole exemestane); xvi) a Mek inhibitor; xvii) a tyrosine kinase inhibitor; xviii) a c-Kit mutant inhibitor, xix) an EGFR inhibitor, a PD-1 inhibitor, or xx) an epigenetic modulator.

In another embodiment, the present disclosure provides a method of treating a cancer in a subject in need thereof by administering to the subject an effective amount of a composition including any one or more compound(s) as described herein in combination with one or more other therapies or medical procedures effective in treating the cancer. Other therapies or medical procedures include suitable anticancer therapy (e.g. drug therapy, vaccine therapy, gene therapy, photodynamic therapy) or medical procedure (e.g. surgery, radiation treatment, hyperthermia heating, bone marrow or stem cell transplant). In one embodiment, the one or more suitable anticancer therapies or medical procedures is selected from treatment with a chemotherapeutic agent (e.g. chemotherapeutic drug), radiation treatment (e.g. x-ray, .gamma.-ray, or electron, proton, neutron, or .alpha, particle beam), hyperthermia heating (e.g. microwave, ultrasound, radiofrequency ablation), Vaccine therapy (e.g. AFP gene hepatocellular carcinoma vaccine, AFP adenoviral vector vaccine, AG-858, allogeneic GM-CSF-secretion breast cancer vaccine, dendritic cell peptide vaccines), gene therapy (e.g. Ad5CMV-p53 vector, adenovector encoding MDA7, adenovirus 5-tumor necrosis factor alpha), photodynamic therapy (e.g. aminolevulinic acid, motexatin lutetium), surgery, or bone marrow and stem cell transplantation.

Embodiment 32(a) of this disclosure relates to the method according to Embodiment 32, wherein the one or more additional therapeutic agents is a PD-1 inhibitor selected from pembrolizumab, nivolumab and emiplimab.

Embodiment 32(a)(1) of this disclosure relates to the method according to Embodiment 32(a), wherein the PD-1 inhibitor is pembrolizumab.

Embodiment 32(a)(2) of this disclosure relates to the method according to Embodiment 32(a), wherein the PD-1 inhibitor is nivolumab.

Embodiment 32(a)(3) of this disclosure relates to the method according to Embodiment 32(a), wherein the PD-1 inhibitor is emiplimab.

Embodiment 32(b) of this disclosure relates to the method according to Embodiment 32, wherein the one or more additional therapeutic agents is a PD-L1 inhibitor selected from atezolizumab, avelumab and durvalumab.

Embodiment 32(b)(1) of this disclosure relates to the method according to Embodiment 32(b), wherein the PD-L1 inhibitor is atezolizumab.

Embodiment 32(b)(2) of this disclosure relates to the method according to Embodiment 32(b), wherein the PD-L1 inhibitor is avelumab.

Embodiment 32(b)(3) of this disclosure relates to the method according to Embodiment 32(b), wherein the PD-L1 inhibitor is durvalumab.

Embodiment 32(c) of this disclosure relates the method according to Embodiment 31, wherein the additional therapeutic agents is a CTLA-4 inhibitor.

Embodiment 32(c)(1) of this disclosure relates the method according to Embodiment 32(c), wherein the CTLA-4 inhibitor is ipilimumab.

### VI. Kits

In another aspect, the present disclosure provides kits that include one or more compounds as described in any one of a compound in one of Embodiments 1-25 (including any subembodiments thereof), or a pharmaceutically acceptable salt, a solvate, a tautomer, a stereoisomer, or a deuterated analog thereof, or a pharmaceutical composition in one of Embodiments 26-27. In some embodiments, the compound or composition is packaged, e.g., in a vial, bottle, flask, which may be further packaged, e.g., within a box, envelope, or bag. The compound or composition may be approved by the U.S. Food and Drug Administration or similar regulatory agency for administration to a mammal, e.g., a human. The compound or composition may be approved for administration to a mammal, e.g., a human, for a KRAS mediated disease or condition. The kits described herein may include written instructions for use and/or other indication that the compound or composition is suitable or approved for administration to a mammal, e.g., a human, for a KRAS mediated disease or condition. The compound or composition may be packaged in unit dose or single dose form, e.g., single dose pills, capsules, or the like.

### VII. Binding Assays

The methods of the present disclosure can involve assays that are able to detect the binding of compounds to a target molecule. Such binding is at a statistically significant level, with a confidence level of at least 90%, or at least 95, 97, 98, 99% or greater confidence level that the assay signal represents binding to the target molecule, i.e., is distinguished from background. In some embodiments, controls are used to distinguish target binding from non-specific binding. A large variety of assays indicative of binding are known for different target types and can be used for this disclosure.

Binding compounds can be characterized by their effect on the activity of the target molecule. Thus, a “low activity” compound has an inhibitory concentration (IC₅₀) or effective concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ or EC₅₀ is defined as the concentration of compound at which 50% of the activity of the target molecule (e.g. enzyme or other protein) activity being measured is lost or gained relative to the range of activity observed when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667 .$$

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121:313-21; Liparoto et al., (1999) Biosensor analysis of the interleukin-2 receptor complex, Journal of Molecular Recognition. 12:316-21; Lipschultz et al., (2000) Experimental design for analysis of complex kinetics using surface plasmon resonance, Methods. 20(3):310-8; Malmqvist., (1999) BIACORE: an affinity biosensor system for characterization of biomolecular interactions, Biochemical Society Transactions 27:335-40; Alfthan, (1998) Surface plasmon resonance biosensors as a tool in antibody engineering, Biosensors & Bioelectronics. 13:653-63; Fivash et al., (1998) BIAcore for macromolecular interaction, Current Opinion in Biotechnology. 9:97-101; Price et al.; (1998) Summary report on the ISOBM TD-4 Workshop: analysis of 56 monoclonal antibodies against the MUC1 mucin. Tumour Biology 19 Suppl 1:1-20; Malmqvist et al, (1997) Biomolecular interaction analysis: affinity biosensor technologies for functional analysis of proteins, Current Opinion in Chemical Biology. 1:378-83; O'Shannessy et al., (1996) Interpretation of deviations from pseudo-first-order kinetic behavior in the characterization of ligand binding by biosensor technology, Analytical Biochemistry. 236:275-83; Malmborg et al., (1995) BIAcore as a tool in antibody engineering, Journal of Immunological Methods. 183:7-13; Van Regenmortel, (1994) Use of biosensors to characterize recombinant proteins, Developments in Biological Standardization. 83:143-51; and O'Shannessy, (1994) Determination of kinetic rate and equilibrium binding constants for macromolecular interactions: a critique of the surface plasmon resonance literature, Current Opinions in Biotechnology. 5:65-71.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g. by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra-high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as described in Gordon, A. J. and Ford, R. A., (1972) The Chemist's Companion: A Handbook Of Practical Data, Techniques, And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistry, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., (1997), Application of Fluorescence Polarization Assays in High-Throughput Screening, Genetic Engineering News, 17:27.

FP is particularly desirable since its readout is independent of the emission intensity (Checovich, W. J., et al., (1995) Nature 375:254-256; Dandliker, W. B., et al., (1981) Methods in Enzymology 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5:77-88.

Fluorophores derived from sphingolipids that may be used in FP assays are commercially available. For example, Molecular Probes (Eugene, Oreg.) currently sells sphingomyelin and one ceramide fluorophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan AG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6:178-182; Mitra et al., (1996) Gene 173:13-17; and Selvin et al., (1995) Meth. Enzymol. 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38:2365-2373; Kahl et al., (1996) Anal. Biochem. 243:282-283; Undenfriend et al., (1987) Anal. Biochem. 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well-known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257:112-119).

General Synthesis

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

The compounds of this disclosure may contain one or more asymmetric or chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, supercritical fluid chromathography, chiral seed crystals, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

It will also be appreciated that in each of the schemes, the addition of any substituent may result in the production of a number of isomeric products (including, but not limited to, enantiomers or one or more diastereomers) any or all of which may be isolated and purified using conventional techniques. When enantiomerically pure or enriched compounds are desired, chiral chromatography and/or enantiomerically pure or enriched starting materials may be employed as conventionally used in the art or as described in the Examples.

Compounds of the present disclosure may be synthesized in accordance with the general reaction schemes and/or examples described below. The general schemes may be altered by substitution of the starting materials with other materials having similar structures to result in corresponding products. The structure of the desired product will generally make apparent to a person of skill in the art the required starting materials.

### Intermediate 3

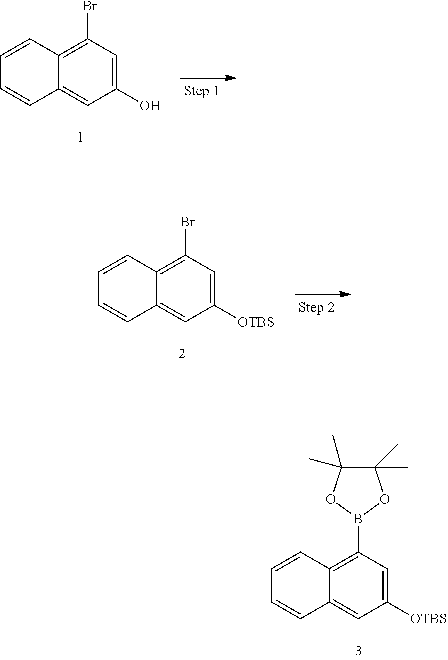 [SMILES: Oc1cc(Br)c2ccccc2c1 ; CC(C)(C)[Si](C)(C)Oc1cc(Br)c2ccccc2c1 ; CC1(C)OB(c2cc(O[Si](C)(C)C(C)(C)C)cc3ccccc23)OC1(C)C]

### Step 1: ((4-bromonaphthalen-2-yl)oxy)(tert-butyl)dimethylsilane (2)

To a solution of 4-bromonaphthalen-2-ol (1, 5.00 g, 22.4 mmol) in 200 mL DCM was added tert-butyl-chloro-dimethyl-silane (5.07 g, 33.6 mmol, 1.5 eq.). The reaction mixture was stirred at ambient temp until all solids dissolved, then imidazole (4.58 g, 67.2 mmol, 3.0 eq.) was added portion-wise to the reaction mixture at ambient temperature while stirring. After 30 min., the reaction mixture was filtered to remove solids, diluted with 150 mL DCM, washed with water and brine, dried over MgSO₄, and concentrated onto silica and was purified by silica gel chromatography eluting with EtOAc/Hexane (product eluted ˜0-5% EtOAc/Hexane). The desired fractions were combined and concentrated to give 4-bromonaphthalen-2-yl)oxy)(tert-butyl)dimethylsilane (2, 7.36 g).

### Step 2: tert-butyldimethyl((4-(4,4,5,5-tetramethyl-1,3,2-dioxaborolan-2-yl)naphthalen-2-yl)oxy)silane (3)

To a solution of ((4-bromonaphthalen-2-yl)oxy)(tert-butyl)dimethylsilane (2, 3.00 g, 8.89 mmol) in 30 mL dioxane was added of 4,4,5,5-tetramethyl-2-(4,4,5,5-tetramethyl-1,3,2-dioxaborolan-2-yl)-1,3,2-dioxaborolane (2.71 g, 10.7 mmol, 1.2 eq.), potassium acetate (1.75 g, 17.8 mmol, 2.0 eq.), and Pd₂(PPh₃)₂C₁₋₂ (0.62 g, 0.889 mmol, 0.1 eq.). The flask was fitted with reflux condenser and stirred under N₂ in a 100° C. pre-heated oil bath. After 2 hr, the reaction mixture was cooled to ambient temp, diluted with 150 mL DCM, filtered to remove insoluble material. The filtrate was washed with water and brine, dried over MgSO₄, and concentrated. The concentrate was dissolved in ˜70 mL DCM and evaporated onto silica and was purified by silica gel chromatography eluting with eluting 0-15% EtOAc/hexane (product eluted ˜10% EtOAc/hexane). The desired fractions were concentrated to give tert-butyldimethyl((4-(4,4,5,5-tetramethyl-1,3,2-dioxaborolan-2-yl)naphthalen-2-yl)oxy)silane (3, 1.98 g).

### Intermediate 6

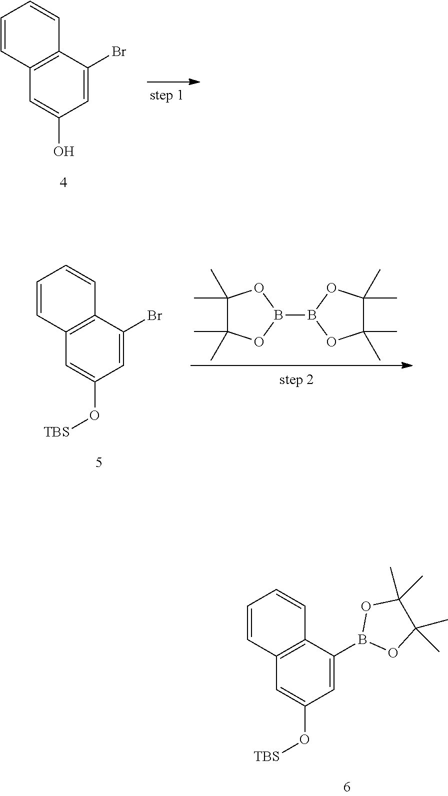

### Step 1: Synthesis of ((4-bromonaphthalen-2-yl)oxy)(tert-butyl)dimethylsilane (5)

4-bromonaphthalen-2-ol (4, 5 g, 22.41 mmol) and tert-butyl-chloro-dimethyl-silane (5.07 g, 33.62 mmol) were dissolved in DCM (200 ml). After stirring for 5 minutes at room temperature, imidazole (4.58 g, 67.24 mmol) was added and the reaction was stirred for 30 minutes. The reaction became cloudy with imidazole salts which were filtered off. The reaction was evaporated on to silica gel and purified by normal phase flash column chromatography to give ((4-bromonaphthalen-2-yl)oxy)(tert-butyl)dimethylsilane (5, 7.17 g).

### Step 2: Synthesis of tert-butyldimethyl((4-(4,4,5,5-tetramethyl-1,3,2-dioxaborolan-2-yl)naphthalen-2-yl)oxy)silane (6)

((4-bromonaphthalen-2-yl)oxy)(tert-butyl)dimethylsilane (5, 7.17 g, 21.26 mmol), 4,4,5,5-tetramethyl-2-(4,4,5,5-tetramethyl-1,3,2-dioxaborolan-2-yl)-1,3,2-dioxaborolane (8.1 g, 31.88 mmol), [1,1′-bis(diphenylphosphino)ferrocene]dichloropalladium(II) (1.56 g, 2.13 mmol), potassium acetate (3.13 g, 31.88 mmol), and dioxane (200 ml) were combined in a 500 ml pressure vial. The vial was purged with nitrogen, sealed, and stirred vigorously at 100° C. for 6 hours. The reaction was poured over brine and extracted with ethyl acetate. The organic layer was dried over anhydrous sodium sulfate, filtered, and evaporated on to silica gel. The product was isolated by normal phase flash column to give tert-butyldimethyl((4-(4,4,5,5-tetramethyl-1,3,2-dioxaborolan-2-yl)naphthalen-2-yl)oxy)silane (6, 7.01 g).

### Example 1

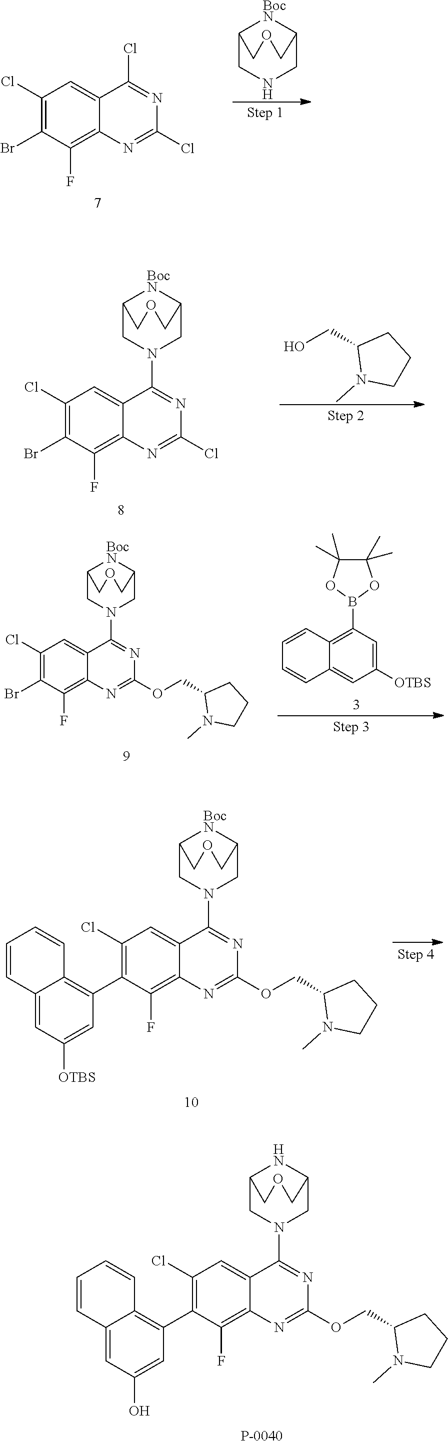

### Step 1: tert-butyl 7-(7-bromo-2,6-dichloro-8-fluoroquinazolin-4-yl)-3-oxa-7,9-diazabicyclo[3.3.1]nonane-9-carboxylate (8)

To a solution of 7-bromo-2,4,6-trichloro-8-fluoro-quinazoline (7, 200 mg, 0.605 mmol) in 3 mL dioxane was added tert-butyl 3-oxa-7,9-diazabicyclo[3.3.1]nonane-9-carboxylate (166 mg, 0.726 mmol, 1.2 eq.) and DIPEA (320 uL, 1.84 mmol, 3.0 eq.). The reaction mixture was allowed to stir at ambient temp. After 2 hr., the reaction mixture was diluted with 20 mL DCM, washed with water, ammonium chloride, and brine, dried over MgSO₄, and concentrated. The crude material was dissolved in DCM and evaporated onto silica and purified by silica gel column chromatography eluting with 0-15% EtOAc/DCM (product eluted ˜5-8% EtOAc/DCM). The desired fractions were pooled and concentrated to give tert-butyl 7-(7-bromo-2,6-dichloro-8-fluoroquinazolin-4-yl)-3-oxa-7,9-diazabicyclo[3.3.1]nonane-9-carboxylate (8, 145 mg). MS (ESI) [M+H⁺]⁺=522.8.

### Step 2: tert-butyl 7-(7-bromo-6-chloro-8-fluoro-2-(((S)-1-methylpyrrolidin-2-yl)methoxy)quinazolin-4-yl)-3-oxa-7,9-diazabicyclo[3.3.1]nonane-9-carboxylate (9)

To a solution of tert-butyl 7-(7-bromo-2,6-dichloro-8-fluoroquinazolin-4-yl)-3-oxa-7,9-diazabicyclo[3.3.1]nonane-9-carboxylate (8, 140 mg, 0.268 mmol) in 2 mL DMSO was added [(2S)-1-methylpyrrolidin-2-yl]methanol (156 mg, 1.35 mmol, 5 eq) and potassium fluoride (32 mg, 0.55 mmol, 2.0 eq). The reaction was stirred in a 120° C. pre-heated oil bath for 3.5 hr., at which time the reaction mixture was cooled to ambient temp., diluted with 25 mL EtOAc, washed with water and brine, dried over MgSO₄, and concentrated. The residue was dissolved in DCM and evaporated onto silica and was purified by silica gel column chromatography eluting with 0-30% MeOH/EtOAc (product elutes ˜5% MeOH/EtOAc). The desired fractions were pooled and concentrated to give tert-butyl 7-(7-bromo-6-chloro-8-fluoro-2-(((S)-1-methylpyrrolidin-2-yl)methoxy)quinazolin-4-yl)-3-oxa-7,9-diazabicyclo[3.3.1]nonane-9-carboxylate (9, 70 mg, 40% yield). MS (ESI) [M+H⁺]⁺=601.8.

### Step 3: tert-butyl 7-(7-(3-((tert-butyldimethylsilyl)oxy)naphthalen-1-yl)-6-chloro-8-fluoro-2-(((S)-1-methylpyrrolidin-2-yl)methoxy)quinazolin-4-yl)-3-oxa-7,9-diazabicyclo[3.3.1]nonane-9-carboxylate (10)

To a solution of tert-butyl 7-(7-bromo-6-chloro-8-fluoro-2-(((S)-1-methylpyrrolidin-2-yl)methoxy)quinazolin-4-yl)-3-oxa-7,9-diazabicyclo[3.3.1]nonane-9-carboxylate (9, 70 mg, 0.107 mmol) in 2 mL dioxane and 0.5 mL H₂O was added tert-butyl-dimethyl-[[4-(4,4,5,5-tetramethyl-1,3,2-dioxaborolan-2-yl)-2-naphthyl]oxy]silane (3, 66 mg, 0.172 mmol, 1.6 eq.), solid Na₂CO₃ (36 mg, 0.34 mmol, 3.2 eq.), and Pd(PPh₃)₄ (15 mg, 0.013 mmol, 0.1 eq.). The reaction mixture was stirred in a 90° C. pre-heated oil bath. After 2 hr., the reaction mixture was cooled to ambient temp, diluted with 25 mL DCM and washed with water. The DCM phase was concentrated, dissolved in 4 mL of ˜25% H₂O/MeCN, and purified by RP-HPLC, eluting with 10-100% MeCN/H₂O (0.1% TFA). The desired fractions were pooled, frozen, and lyophilized to give tert-butyl 7-(7-(3-((tert-butyldimethylsilyl)oxy)naphthalen-1-yl)-6-chloro-8-fluoro-2-(((S)-1-methylpyrrolidin-2-yl)methoxy)quinazolin-4-yl)-3-oxa-7,9-diazabicyclo[3.3.1]nonane-9-carboxylate (10, 40 mg). MS (ESI) [M+H⁺]⁺=778.5.

### Step 4: 4-(4-(3-oxa-7,9-diazabicyclo[3.3.1]nonan-7-yl)-6-chloro-8-fluoro-2-(((S)-1-methylpyrrolidin-2-yl)methoxy)quinazolin-7-yl)naphthalen-2-ol (P-0040)

To a solution of tert-butyl 7-[7-[3-[tert-butyl(dimethyl)silyl]oxy-1-naphthyl]-6-chloro-8-fluoro-2-[[(2S)-1-methylpyrrolidin-2-yl]methoxy]quinazolin-4-yl]-3-oxa-7,9-diazabicyclo[3.3.1]nonane-9-carboxylate (10, 39 mg, 0.50 mmol) in 0.5 mL MeOH was added 3 ml of 4N HCl/dioxane. The reaction mixture stirred at ambient temp. After for 90 min, the reaction mixture was concentrated to a yellow residue, dissolved in 2 mL H₂O and purified by RP silica gel column chromatography eluting with MeCN/H₂O (0.1% formic acid). The desired fractions were pooled, frozen, and lyophilized to white solid: 4-(4-(3-oxa-7,9-diazabicyclo[3.3.1]nonan-7-yl)-6-chloro-8-fluoro-2-(((S)-1-methylpyrrolidin-2-yl)methoxy)quinazolin-7-yl)naphthalen-2-ol (P-0040, 18 mg) as a bis-HCl salt. MS (ESI) [M+H⁺]⁺=564.0.

### Example 2

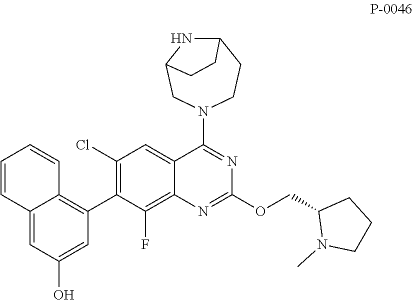 [SMILES: CN1CCC[C@H]1COc1nc(N2CCC3CCC(C2)N3)c2cc(Cl)c(-c3cc(O)cc4ccccc34)c(F)c2n1]

### 4-(4-(3,9-diazabicyclo[4.2.1]nonan-3-yl)-6-chloro-8-fluoro-2-(((S)-1-methylpyrrolidin-2-yl)methoxy)quinazolin-7-yl)naphthalen-2-ol (P-0046)

All synthetic steps were performed as detailed in Example 1, by substitution of tert-butyl 3,9-diazabicyclo[4.2.1]nonane-9-carboxylate in synthetic Step 1 to afford 4-(4-(3,9-diazabicyclo[4.2.1]nonan-3-yl)-6-chloro-8-fluoro-2-(((S)-1-methylpyrrolidin-2-yl)methoxy)quinazolin-7-yl)naphthalen-2-ol (P-0046). MS (ESI) [M+H⁺]⁺=563.0.

### Example 3

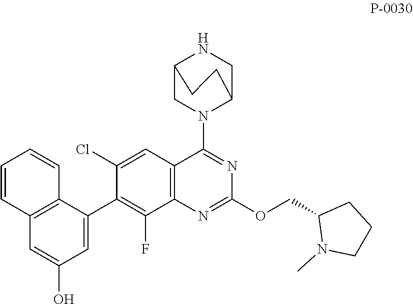 [SMILES: CN1CCC[C@H]1COc1nc(N2CC3CCC2CN3)c2cc(Cl)c(-c3cc(O)cc4ccccc34)c(F)c2n1]

### 4-(4-(2,5-diazabicyclo[2.2.2]octan-2-yl)-6-chloro-8-fluoro-2-(((S)-1-methylpyrrolidin-2-yl)methoxy)quinazolin-7-yl)naphthalen-2-ol (P-0030)

All synthetic steps were performed as detailed in Example 1, by substitution of tert-butyl 2,5-diazabicyclo[2.2.2]octane-2-carboxylate in synthetic Step 1 to afford 4-(4-(2,5-diazabicyclo[2.2.2]octan-2-yl)-6-chloro-8-fluoro-2-(((S)-1-methylpyrrolidin-2-yl)methoxy)quinazolin-7-yl)naphthalen-2-ol (P-0030). MS (ESI) [M+H⁺]⁺=548.3.

### Example 4

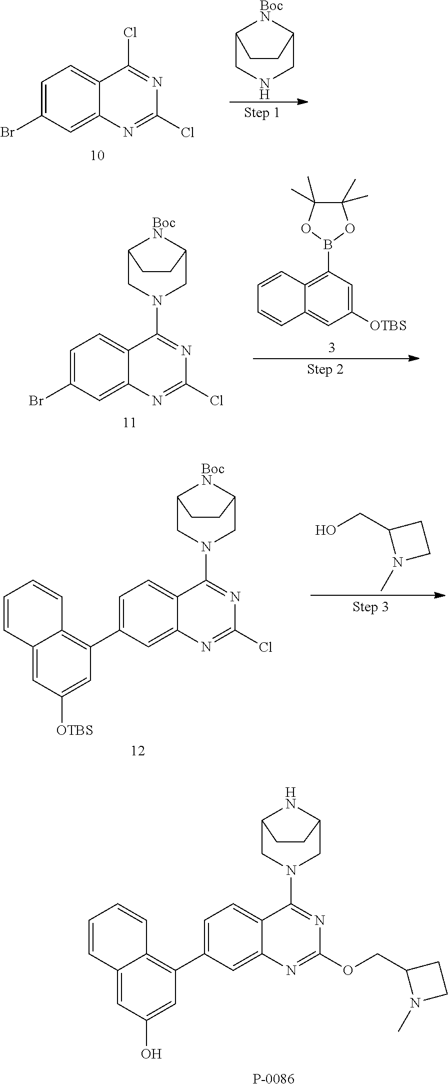

### Step 1: tert-butyl (1R,5S)-3-(7-bromo-2-chloroquinazolin-4-yl)-3,8-diazabicyclo[3.2.1]octane-8-carboxylate (11)

To a solution of 7-bromo-2,4-dichloro-quinazoline (10, 500 mg, 1.80 mmol) in 7 mL dioxane was added tert-butyl (1R,5S)-3,8-diazabicyclo[3.2.1]octane-8-carboxylate (460 mg, 2.17 mmol, 1.2 eq.) and DIEA (1.0 mL, 5.7 mmol, 3.2 eq.). The reaction was purged with N₂ and allowed to stir at ambient temp. After overnight stir, the reaction mixture was diluted with 40 mL DCM, washed with water and brine, dried over MgSO₄, and concentrated. The resulting reaction mixture was dissolved in DCM, evaporated onto silica and was purified by silica gel column chromatography eluting with 0-30% EtOAc/DCM (product eluted at ˜10% EtOAc/DCM). The desired fractions were pooled and concentrated to give tert-butyl (1R,5S)-3-(7-bromo-2-chloroquinazolin-4-yl)-3,8-diazabicyclo[3.2.1]octane-8-carboxylate (11, 578 mg). LCMS MS (ESI) [M+H⁺]⁺=454.7.

### Step 2: tert-butyl (1R,5S)-3-(7-(3-((tert-butyldimethylsilyl)oxy)naphthalen-1-yl)-2-chloroquinazolin-4-yl)-3,8-diazabicyclo[3.2.1]octane-8-carboxylate_(12)

To a solution of tert-butyl (1R,5S)-3-(7-bromo-2-chloroquinazolin-4-yl)-3,8-diazabicyclo[3.2.1]octane-8-carboxylate (11, 1.83 g, 4.03 mmol) in 28 mL dioxane and 7 mL H₂O was added, tert-butyl-dimethyl-[[4-(4,4,5,5-tetramethyl-1,3,2-dioxaborolan-2-yl)-2-naphthyl]oxy]silane (3, 2.02 g, 5.26 mmol, 1.3 eq.), solid K₂CO₃ (1.96 g, 14.2 mmol, 3.5 eq), and Pd(PPh₃)₄ (933 mg (0.807 mmol, 0.2 eq.). The reaction mixture was stirred in a 80° C. pre-heated oil bath. After 1 hr., the reaction mixture was cooled to ambient temp, diluted with 200 mL DCM, washed with water and brine, dried over MgSO₄, and concentrated. The crude material was dissolved in DCM, evaporated onto silica and purified by silica gel column chromatography, eluting with 0-25% EtOAc/Hexane (the product eluted at ˜10% EtOAc/hexane). The desired fractions were pooled and concentrated to give tert-butyl (1R,5S)-3-(7-(3-((tert-butyldimethylsilyl)oxy)naphthalen-1-yl)-2-chloroquinazolin-4-yl)-3,8-diazabicyclo[3.2.1]octane-8-carboxylate (12, 1.86 g). MS (ESI) [M+H⁺]⁺=632.1.

### Step 3: 4-(4-((1R,5S)-3,8-diazabicyclo[3.2.1]octan-3-yl)-2-((1-methylazetidin-2-yl)methoxy)quinazolin-7-yl)naphthalen-2-ol (P-0086)

To a solution of tert-butyl (1R,5S)-3-(7-(3-((tert-butyldimethylsilyl)oxy)naphthalen-1-yl)-2-chloroquinazolin-4-yl)-3,8-diazabicyclo[3.2.1]octane-8-carboxylate (12, 100 mg, 0.158 mmol) in 1.0 mL dioxane was added (1-methylazetidin-2-yl)methanol (160 mg, 1.59 mmol, 10 eq.), solid K₂CO₃ (220 mg, 1.59 mmol, 10 eq.) and RuPhos Pd G4 catalyst (40 mg, 0.047 mmol, 0.3 eq.). The reaction mixture was stirred under N₂ in a 95° C. pre-heated sand bath. After 30 min, the reaction mixture was filtered to remove insoluble material, concentrated, dissolved in DCM, and evaporated onto silica. The product was purified by silica gel column chromatography, eluting with EtOAc/DCM to remove most by-product peaks, then MeOH/EtOAc to elute protected product (protected product eluted ˜15% MeOH/EtOAc). The desired fractions were pooled and concentrated, and then dissolved in 0.5 mL MeOH and 3 mL of 4N HCl/dioxane. The reaction mixture was stirred at ambient temp for 1 hr., at which time the reaction mixture was concentrated, dissolved in 2 mL of ˜10% MeCN/H₂O, and purified by RP-flash silica gel column chromatography eluting with MeCN/H₂O (0.1% formic acid). The desired fractions were pooled, frozen, and lyophilized to give 4-(4-((1R,5S)-3,8-diazabicyclo[3.2.1]octan-3-yl)-2-((1-methylazetidin-2-yl)methoxy)quinazolin-7-yl)naphthalen-2-ol (P-0086, 21 mg, 24% yield) as a bis-HCl salt. MS (ESI) [M+H⁺]⁺=482.2.

### Example 5

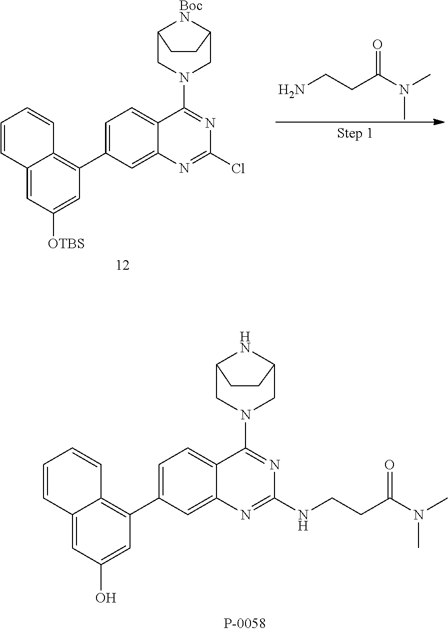

### Step 1: 3-((4-((1R,5S)-3,8-diazabicyclo[3.2.1]octan-3-yl)-7-(3-hydroxynaphthalen-1-yl)quinazolin-2-yl)amino)-N,N-dimethylpropanamide (P-0058)

To a solution of tert-butyl (1R,5S)-3-(7-(3-((tert-butyldimethylsilyl)oxy)naphthalen-1-yl)-2-chloroquinazolin-4-yl)-3,8-diazabicyclo[3.2.1]octane-8-carboxylate (12, 50 mg, 0.079 mmol) (10) in 0.5 mL dioxane was added 3-amino-N,N-dimethyl-propanamide (173 mg, 1.49 mmol, 19 eq.), and solid K₂CO₃ (214 mg, 1.55 mmol, 20 eq.). The reaction mixture was stirred under N₂ in a pre-heated 100° C. oil bath. After 1 hr., the reaction mixture was cooled to ambient temp and filtered over glass fiber pad to remove insoluble material. To the filtrate was added 0.3 mL MeOH and 2 mL of 2N HCl/dioxane. After stirring at ambient temp for 1 hr., the reaction mixture was concentrated to residue, dissolved in 4 mL of ˜10% MeCN/H₂O and purified by prep-RP HPLC, eluting with H₂O/MeCN (0.1% TFA). The desired fractions were pooled, frozen, and lyophilized. The lyophilized product was dissolved in MeOH and passed through a 200 mg SPE-HCO₃ column for neutralization. The filtrate was concentrated and dried overnight in vacuum oven (50° C.) to give 3-((4-((1R,5S)-3,8-diazabicyclo[3.2.1]octan-3-yl)-7-(3-hydroxynaphthalen-1-yl)quinazolin-2-yl)amino)-N,N-dimethylpropanamide (P-0058, 9.1 mg). MS (ESI) [M+H⁺]⁺=497.6.

### Example 6

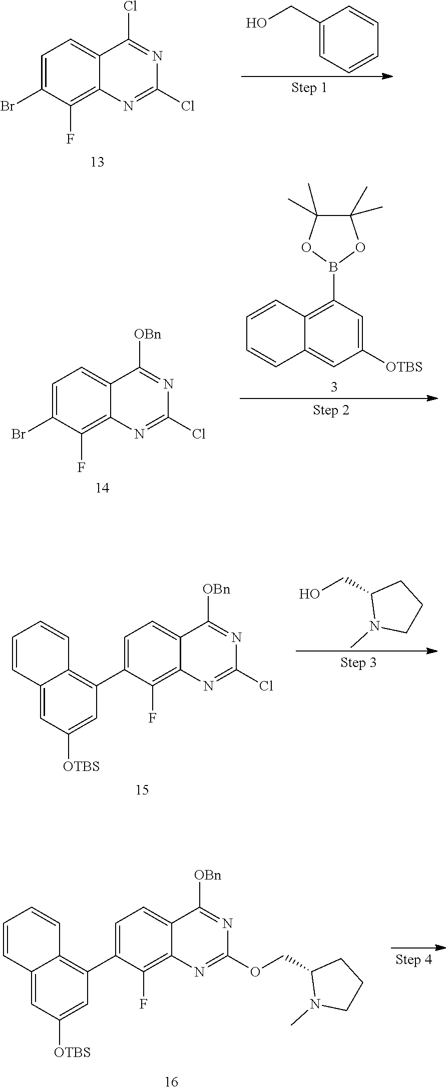

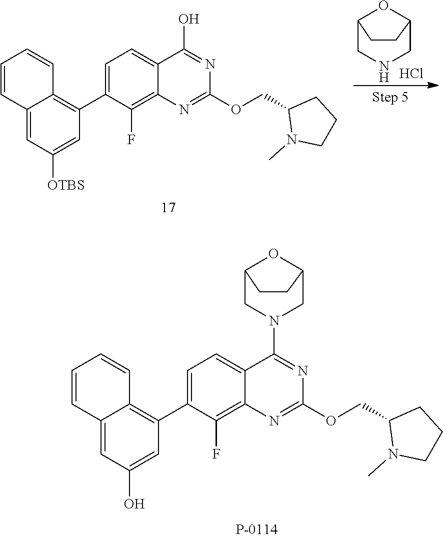 [SMILES: CN1CCC[C@H]1COc1nc(O)c2ccc(-c3cc(O[Si](C)(C)C(C)(C)C)cc4ccccc34)c(F)c2n1 ; C1CC2CNCC1O2 ; Cl ; CN1CCC[C@H]1COc1nc(N2CC3CCC(C2)O3)c2ccc(-c3cc(O)cc4ccccc34)c(F)c2n1]

### Step 1: 4-(benzyloxy)-7-bromo-2-chloro-8-fluoroquinazoline (14)

To a solution of 7-bromo-2,4-dichloro-quinazoline (13, 2.00 g, 7.20 mmol) in 15 mL of t-butanol and 5 mL THF was added benzyl alcohol (0.92 mL, 8.9 mmol, 1.3 eq.). The suspension was stirred under N₂ followed by addition of crushed KOH (418 mg, 7.43 mmol, 1.1 eq.) in one portion. The suspension stirred at ambient temp. After 2 hr., the reaction mixture was diluted with 200 mL EtOAc, washed with water and ammonium chloride. The organic layer was concentrated, dissolved in DCM, and evaporated onto silica. The product was purified by RP silica gel column chromatography eluting with MeCN/H₂O (0.1% formic acid). The desired fractions were pooled, concentrated to ˜50 mL, frozen, and lyophilized to give 4-(benzyloxy)-7-bromo-2-chloro-8-fluoroquinazoline (14, 1.73 g). MS (ESI) [M+H⁺]⁺=368.8.

### Step 2: 4-(benzyloxy)-7-(3-((tert-butyldimethylsilyl)oxy)naphthalen-1-yl)-2-chloro-8-fluoroquinazoline (15)

To a solution of 4-(benzyloxy)-7-bromo-2-chloro-8-fluoroquinazoline (14, 1.28 g, 3.58 mmol) in 32 mL dioxane and 8 mL H₂O, was added tert-butyl-dimethyl-[[4-(4,4,5,5-tetramethyl-1,3,2-dioxaborolan-2-yl)-2-naphthyl]oxy]silane (3, 1.35 g, 3.48 mmol, 1.0 eq.), solid K₂CO₃ (1.29 g, 12.2 mmol, 3.5 eq.) and Pd(PPh₃)₄ (0.81 g, 0.70 mmol, 0.2 eq.). The reaction mixture was stirred under N₂ in an 80° C. pre-heated oil bath. After 1 hr, the reaction mixture was cooled to ambient temp, diluted with 200 mL DCM, washed with water and brine, dried over MgSO₄, and concentrated. The crude material was dissolved in DCM, evaporated onto silica and purified by silica gel column chromatography eluting with EtOAc/hexane (the product eluted at 10-15% EtOAc/hexane). The fractions containing desired product were pooled and concentrated to give 4-(benzyloxy)-7-(3-((tert-butyldimethylsilyl)oxy)naphthalen-1-yl)-2-chloro-8-fluoroquinazoline (15, 0.34 g). MS (ESI) [M+H⁺]⁺=545.2.

### Step 3: (S)-4-(benzyloxy)-7-(3-((tert-butyldimethylsilyl)oxy)naphthalen-1-yl)-8-fluoro-2-((1-methylpyrrolidin-2-yl)methoxy)quinazoline (16)

To a solution of 4-(benzyloxy)-7-(3-((tert-butyldimethylsilyl)oxy)naphthalen-1-yl)-2-chloro-8-fluoroquinazoline (15, 340 mg, 0.624 mmol) in 4 mL dioxane was added [(2S)-1-methylpyrrolidin-2-yl]methanol (362 mg, 3.14 mmol, 5 eq.), solid K₂CO₃ (518 mg, 3.75 mmol, 6 eq.), and RuPhos Pd G4 catalyst (106 mg, 0.125 mmol, 0.2 eq.). The reaction mixture was stirred under N₂ in a 90° C. oil bath. After 1 hr, the reaction mixture was filtered to remove insoluble material, concentrated to a residue, dissolved in DCM, and concentrated onto silica. The product was purified by silica gel column chromatography eluting with EtOAc/DCM (product eluted at 70% EtOAc/DCM). The desired fractions were pooled and concentrated to give (S)-4-(benzyloxy)-7-(3-((tert-butyldimethylsilyl)oxy)naphthalen-1-yl)-8-fluoro-2-((1-methylpyrrolidin-2-yl)methoxy)quinazoline (16, 236 mg). MS (ESI) [M+H⁺]⁺=625.1.

### Step 4: (S)-7-(3-((tert-butyldimethylsilyl)oxy)naphthalen-1-yl)-8-fluoro-2-((1-methylpyrrolidin-2-yl)methoxy)quinazolin-4-ol (17)

A solution of (S)-4-(benzyloxy)-7-(3-((tert-butyldimethylsilyl)oxy)naphthalen-1-yl)-8-fluoro-2-((1-methylpyrrolidin-2-yl)methoxy)quinazoline (16, 202 mg, 0.627 mmol) in 15 mL THF and 100 mL MeOH was passed through an HCube hydrogenator at 60° C. using Pd/C cartridge. After one pass, LCMS indicated ˜90% conversion to desired hydroxy and ˜10% remaining O-Bn start material. The material was concentrated to give (S)-7-(3-((tert-butyldimethylsilyl)oxy)naphthalen-1-yl)-8-fluoro-2-((1-methylpyrrolidin-2-yl)methoxy)quinazolin-4-ol (17, 171 mg). MS (ESI) [M+H⁺]⁺=535.0.

### Step 5: 4-(4-((1R,5S)-8-oxa-3-azabicyclo[3.2.1]octan-3-yl)-8-fluoro-2-(((S)-1-methylpyrrolidin-2-yl)methoxy)quinazolin-7-yl)naphthalen-2-ol (P-0115)

To solution of (S)-7-(3-((tert-butyldimethylsilyl)oxy)naphthalen-1-yl)-8-fluoro-2-((1-methylpyrrolidin-2-yl)methoxy)quinazolin-4-ol (17, 81 mg, 0.15 mmol) in 5 mL DCM was added DIPEA (0.20 mL, 1.15 mmol). The reaction mixture was purged with N₂ and stirred in −40° C. bath. While stirring cold, triflic anhydride (35 uL, 0.21 mmol, 1.4 eq.) was added dropwise. After ˜20 min, a solution of 8-oxa-3-azabicyclo[3.2.1]octane hydrochloride (69 mg, 0.46 mmol, 3 eq.) and DIPEA (0.3 mL, 1.72 mmol) in 3 mL DCM was added slowly to the cold (−40° C.) stirring triflate solution. Following addition, the cold bath was removed and the reaction allowed to warm to ambient temp. After 1 hr., the reaction was concentrated. To the residue was added 3 mL of 4N HCl/dioxane and 0.5 mL MeOH and the reaction mixture stirred at ambient temp. After 1 hr., the reaction mixture was concentrated, dissolved in 4 ml of ˜75% MeCN/H₂O, and injected onto prep-LC for purification, eluting with MeCN/H₂O (0.1% TFA). The desired fractions were pooled, frozen, and lyophilized to give 4-(4-((1R,5S)-8-oxa-3-azabicyclo[3.2.1]octan-3-yl)-8-fluoro-2-(((S)-1-methylpyrrolidin-2-yl)methoxy)quinazolin-7-yl)naphthalen-2-ol (P-0115, 11.0 mg) as TFA salt. MS (ESI) [M+H⁺]⁺=515.1.

### Intermediate 18

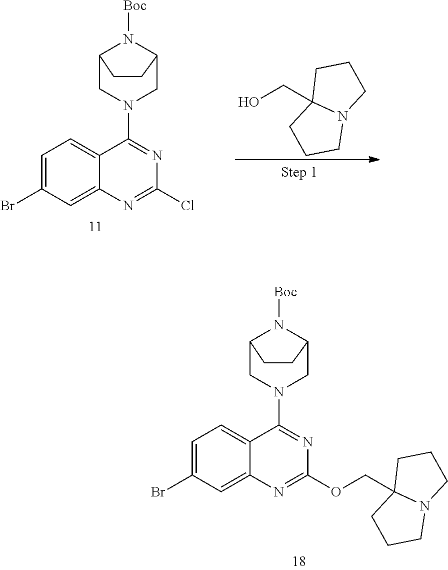 [SMILES: CC(C)(C)OC(=O)N1C2CCC1CN(c1nc(Cl)nc3cc(Br)ccc13)C2 ; OCC12CCCN1CCC2 ; CC(C)(C)OC(=O)N1C2CCC1CN(c1nc(OCC34CCCN3CCC4)nc3cc(Br)ccc13)C2]

### Step 1: Tert-butyl (1R,5S)-3-(7-bromo-2-((tetrahydro-1H-pyrrolizin-7a(5H)-yl)methoxy)quinazolin-4-yl)-3,8-diazabicyclo[3.2.1]octane-8-carboxylate (18)

Tert-butyl (1R,5S)-3-(7-bromo-2-((tetrahydro-1H-pyrrolizin-7a(5H)-yl)methoxy)quinazolin-4-yl)-3,8-diazabicyclo[3.2.1]octane-8-carboxylate (11, 1.51 g, 3.33 mmol), hexahydro-1h-pyrrolizin-7a-ylmethanol (1.41 g, 9.98 mmol), KF (0.39 g, 6.66 mmol), and DMSO (50 ml) were combined in a 100 ml round bottom flask. The reaction was stirred at 120° C. for 24 horns in an oil bath. The reaction was diluted with water and extracted with DCM. The organic layer was dried over anhydrous sodium sulfate, filtered and then evaporated on to silica gel. The product was isolated by reverse phase flash column chromatography (120 g, C18, water:ACN) to give tert-butyl (1R,5S)-3-(7-bromo-2-((tetrahydro-1H-pyrrolizin-7a(5H)-yl)methoxy)quinazolin-4-yl)-3,8-diazabicyclo[3.2.1]octane-8-carboxylate (18, 0.554 g).

### Intermediate 20

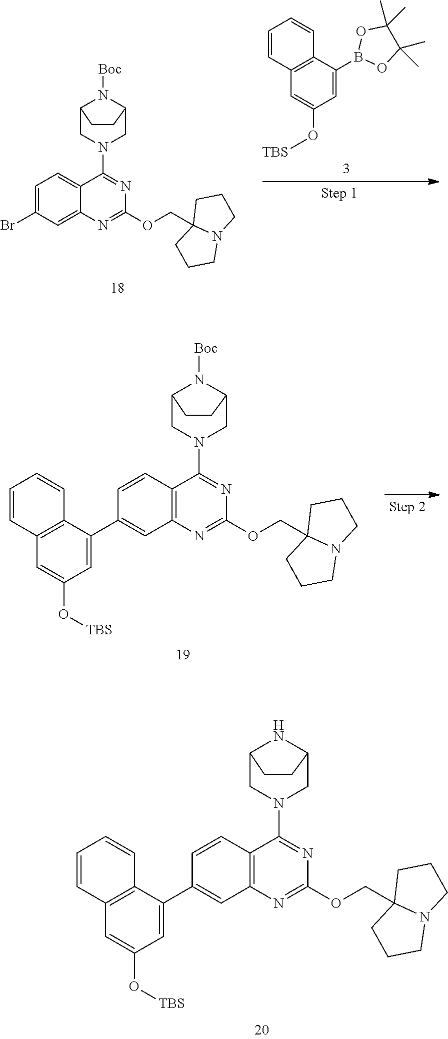

### Step 1: Tert-butyl (1R,5S)-3-(7-(3-((tert-butyldimethylsilyl)oxy)naphthalen-1-yl)-2-((tetrahydro-1H-pyrrolizin-7a(5H)-yl)methoxy)quinazolin-4-yl)-3,8-diazabicyclo[3.2.1]octane-8-carboxylate (19)

Tert-butyl (1R,5S)-3-(7-bromo-2-((tetrahydro-1H-pyrrolizin-7a(5H)-yl)methoxy)quinazolin-4-yl)-3,8-diazabicyclo[3.2.1]octane-8-carboxylate (18, 0.48 g, 0.86 mmol), tert-butyldimethyl((4-(4,4,5,5-tetramethyl-1,3,2-dioxaborolan-2-yl)naphthalen-2-yl)oxy)silane (3, 0.5 g, 1.29 mmol), [1,1′-bis(diphenylphosphino)ferrocene]dichloropalladium(II) (0.06 g, 0.09 mmol), 2.5M potassium carbonate (1.03 ml), and dioxane (7 ml) were combined in a 20 ml microwave vial. The vial was sealed and stirred under nitrogen atmosphere at 100° C. for 6 hours. The reaction was poured over brine and extracted with ethyl acetate. The organic layer was dried over anhydrous sodium sulfate, filtered and evaporated on to silica gel. The product was isolated by reverse phase flash column chromatography (40 g, C18) to give tert-butyl (1R,5S)-3-(7-(3-((tert-butyldimethylsilyl)oxy)naphthalen-1-yl)-2-((tetrahydro-1H-pyrrolizin-7a(5H)-yl)methoxy)quinazolin-4-yl)-3,8-diazabicyclo[3.2.1]octane-8-carboxylate (19, 0.503 g).

### Step 2: 4-((1R,5S)-3,8-diazabicyclo[3.2.1]octan-3-yl)-7-(3-((tert-butyldimethylsilyl)oxy)naphthalen-1-yl)-2-((tetrahydro-1H-pyrrolizin-7a(5H)-yl)methoxy)quinazoline (20)

Tert-butyl (1R,5S)-3-(7-(3-((tert-butyldimethylsilyl)oxy)naphthalen-1-yl)-2-((tetrahydro-1H-pyrrolizin-7a(5H)-yl)methoxy)quinazolin-4-yl)-3,8-diazabicyclo[3.2.1]octane-8-carboxylate (19, 0.5 g, 0.68 mmol) was dissolved in DCM (50 ml). The reaction was cooled to 0° C. and a mixture of TFA (2.52 ml, 33.97 mmol) in DCM (7.5 ml) was added slowly and the reaction was stirred at 0° C. for 6 hours. The reaction was evaporated to dryness without submerging in a water bath. The reaction was then diluted with ethyl acetate (100 ml) and washed with saturated sodium bicarbonate. The organic layer was dried over anhydrous sodium sulfate, filtered and then evaporated to dryness to give 4-((1R,5S)-3,8-diazabicyclo[3.2.1]octan-3-yl)-7-(3-((tert-butyldimethylsilyl)oxy)naphthalen-1-yl)-2-((tetrahydro-1H-pyrrolizin-7a(5H)-yl)methoxy)quinazoline (20, 0.437 g).

### Example 7

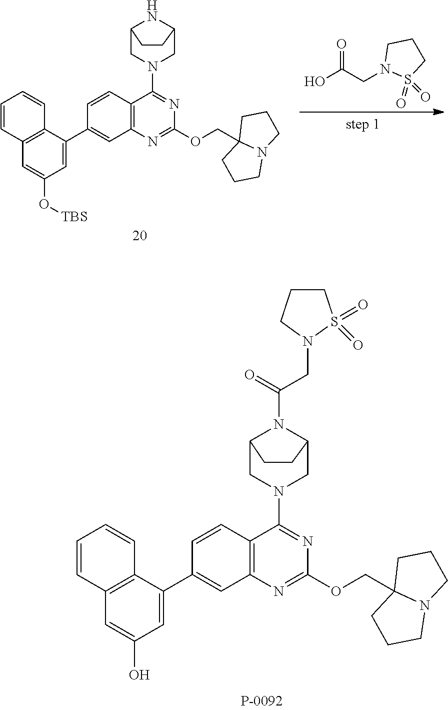

### Step 1: 2-(1,1-dioxidoisothiazolidin-2-yl)-1-((1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)ethan-1-one (P-0092)

Tert-butyl-[[4-[4-(3,8-diazabicyclo[3.2.1]octan-3-yl)-2-(1,2,3,5,6,7-hexahydropyrrolizin-8-ylmethoxy)quinazolin-7-yl]-2-naphthyl]oxy]-dimethyl-silane (20, 0.03 g, 0.05 mmol), 2-(1,1-dioxo-1,2-thiazolidin-2-yl)acetic acid (0.04 g, 0.24 mmol), HATU (0.02 g, 0.14 mmol), DMF (2 ml), and DIEA (0.04 ml, 0.24 mmol) were combined in a 20 ml scintillation vial and the stirred at room temperature for 2 hours. The reaction was treated with TFA (0.07 ml, 1.0 mmol) and heated to 60° C. for 2 hours. The reaction was then concentrated, diluted with 95/5 water/ACN and purified by reverse phase flash column chromatography to give 2-(l, 1-dioxidoisothiazolidin-2-yl)-1-((1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)ethan-1-one (P-0092, 0.008 g). MS (ESI) [M+H⁺]⁺=683.65.

### Example 8

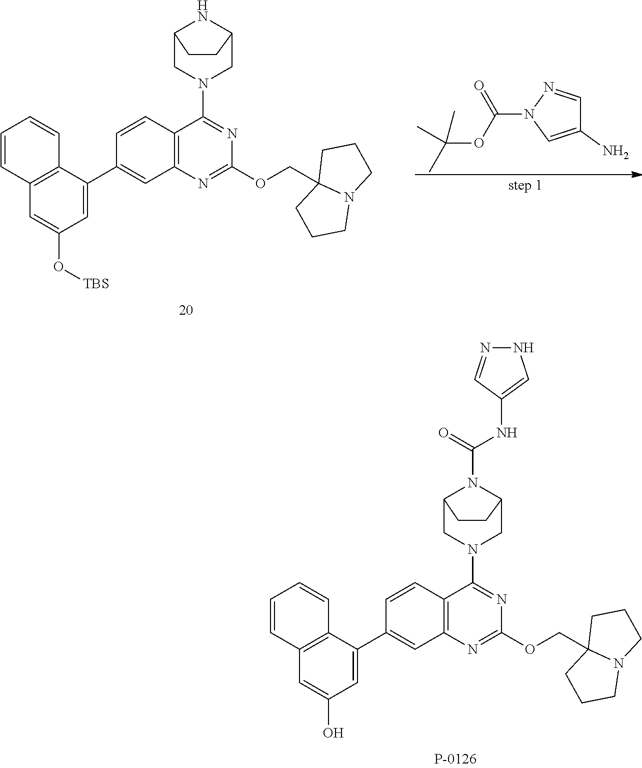

### Step 1: (1R,5S)-3-(7-(3-hydroxynaphthalen-1-yl)-2-((tetrahydro-1H-pyrrolizin-7a(5H)-yl)methoxy)quinazolin-4-yl)-N-(1H-pyrazol-4-yl)-3,8-diazabicyclo[3.2.1]octane-8-carboxamide (P-0126)

Tert-butyl-[[4-[4-(3,8-diazabicyclo[3.2.1]octan-3-yl)-2-(1,2,3,5,6,7-hexahydropyrrolizin-8-ylmethoxy)quinazolin-7-yl]-2-naphthyl]oxy]-dimethyl-silane (20, 0.03 g, 0.05 mmol) was dissolved in dioxane (2 ml) and DIEA (0.04 ml, 0.24 mmol) and stirred at room temperature, bis(trichloromethyl) carbonate (0.01 g, 0.024 mmol) in DCM (1 ml) was added dropwise and the reaction was stirred for 60 minutes. Formation of the amide chloride intermediate was confirmed by LCMS in MeOH. To the reaction mixture was added tert-butyl 4-aminopyrazole-1-carboxylate (0.01 ml, 0.07 mmol) and K₂CO₃ (0.02 g, 0.14 mmol) and the reaction was heated to 70° C. for 2 hours. The reaction was then cooled to room temperature, filtered to remove solids, then treated with 4M HCl (1.2 ml) in dioxane and stirred at room temperature for 2 hours. The de-protected product was isolated by prep-HPLC to give (1R,5S)-3-(7-(3-hydroxynaphthalen-1-yl)-2-((tetrahydro-1H-pyrrolizin-7a(5H)-yl)methoxy)quinazolin-4-yl)-N-(1H-pyrazol-4-yl)-3,8-diazabicyclo[3.2.1]octane-8-carboxamide (P-0126, 0.012 g). MS (ESI) [M+H⁺]⁺=631.20.

### Example 9

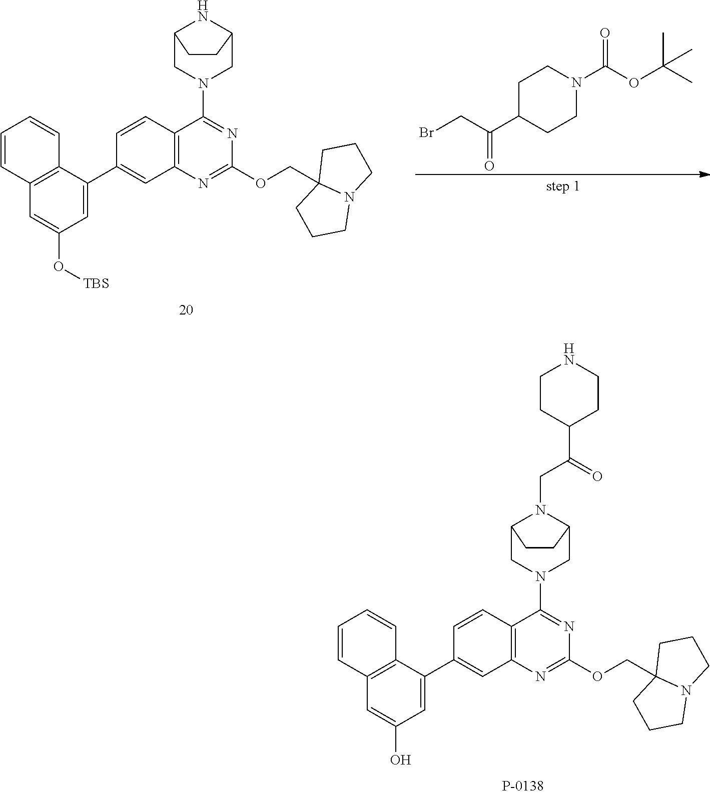

### 2-((1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)-1-(piperidin-4-yl)ethan-1-one (P-0138)

Tert-butyl-[[4-[4-(3,8-diazabicyclo[3.2.1]octan-3-yl)-2-(1,2,3,5,6,7-hexahydropyrrolizin-8-ylmethoxy)quinazolin-7-yl]-2-naphthyl]oxy]-dimethyl-silane (20, 0.03 g, 0.05 mmol) was dissolved in THF (2 ml) and TEA (0.03 ml, 0.24 mmol), tert-butyl 4-(2-bromoacetyl)piperidine-1-carboxylate (0.02 ml, 0.07 mmol) was added and the reaction was stirred at 70° C. for 2 hours. The reaction was cooled to room temperature, treated with 4M HCl (0.59 ml), and stirred for 30 minutes at 70° C. The product was isolated by prep-HPLC to give 2-((1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)-1-(piperidin-4-yl)ethan-1-one (P-0138, 0.014 g. MS (ESI) [M+H⁺]⁺=647.30.

### Example 10

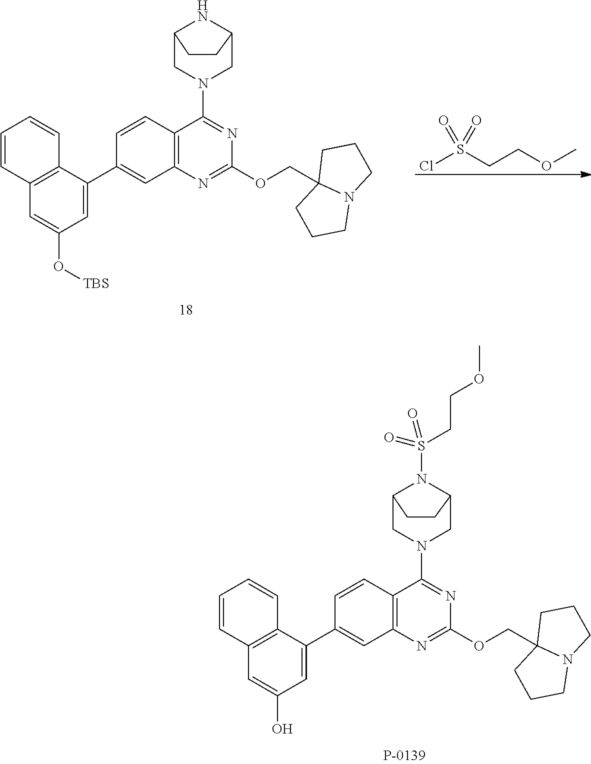

### Step 1: 4-(4-((1R,5S)-8-((2-methoxyethyl)sulfonyl)-3,8-diazabicyclo[3.2.1]octan-3-yl)-2-((tetrahydro-1H-pyrrolizin-7a(5H)-yl)methoxy)quinazolin-7-yl)naphthalen-2-ol (P-0139)

Tert-butyl-[[4-[4-(3,8-diazabicyclo[3.2.1]octan-3-yl)-2-(1,2,3,5,6,7-hexahydropyrrolizin-8-ylmethoxy)quinazolin-7-yl]-2-naphthyl]oxy]-dimethyl-silane (20, 0.03 g, 0.06 mmol), 2-methoxyethanesulfonyl chloride (0.01 g, 0.07 mmol), THF (2 ml), and DIEA (0.02 ml, 0.12 mmol) were combined in a 20 ml scintillation vial was stirred at room temperature for 2 hours. TFA (0.21 ml, 2.88 mmol) was added and the reaction was heated to 70° C. for 2 hours. The product was isolated by prep-HPLC to give 4-(4-((1R,5S)-8-((2-methoxyethyl)sulfonyl)-3,8-diazabicyclo[3.2.1]octan-3-yl)-2-((tetrahydro-1H-pyrrolizin-7a(5H)-yl)methoxy)quinazolin-7-yl)naphthalen-2-ol (P-0139, 0.009 g). MS (ESI) [M+H⁺]⁺=644.25.

### Example 11

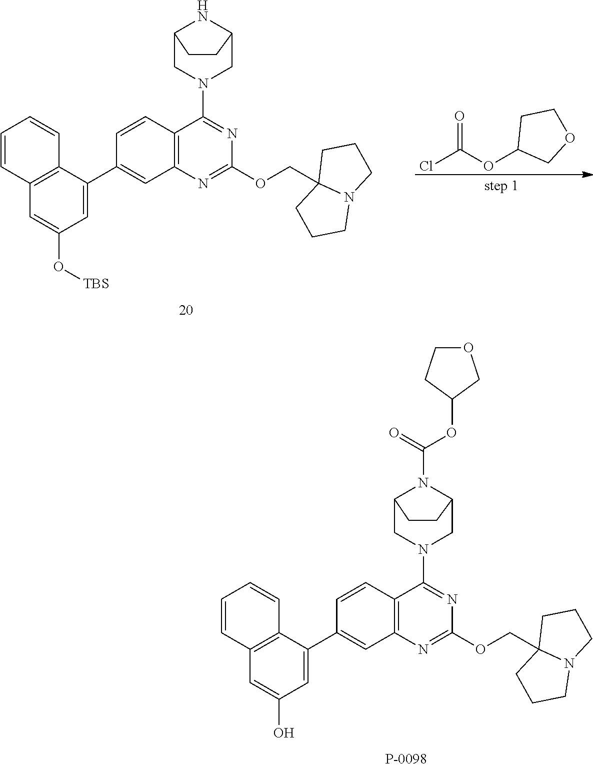

### Step 1: Tetrahydrofuran-3-yl (1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octane-8-carboxylate (P-0098)

Tert-butyl-[[4-[4-(3,8-diazabicyclo[3.2.1]octan-3-yl)-2-(1,2,3,5,6,7-hexahydropyrrolizin-8-ylmethoxy)quinazolin-7-yl]-2-naphthyl]oxy]-dimethyl-silane (0.03 g, 0.06 mmol), tetrahydrofuran-3-yl carbonochloridate (20, 0.01 g, 0.06 mmol), THF (2 ml), and DIEA (0.02 ml, 0.12 mmol) were combined in a 20 ml scintillation vial was stirred at room temperature for 2 hours. TFA (0.21 ml, 2.88 mmol) was added and the reaction was heated to 70° C. for 2 hours. The product was isolated by prep-HPLC to give tetrahydrofuran-3-yl(1R,5S)-3-(7-(3-hydroxynaphthalen-1-yl)-2-((tetrahydro-1H-pyrrolizin-7a(5H)-yl)methoxy)quinazolin-4-yl)-3,8-diazabicyclo[3.2.1]octane-8-carboxylate (P-0098, 0.021 g). MS (ESI) [M+H⁺]⁺=636.25.

### Example 12

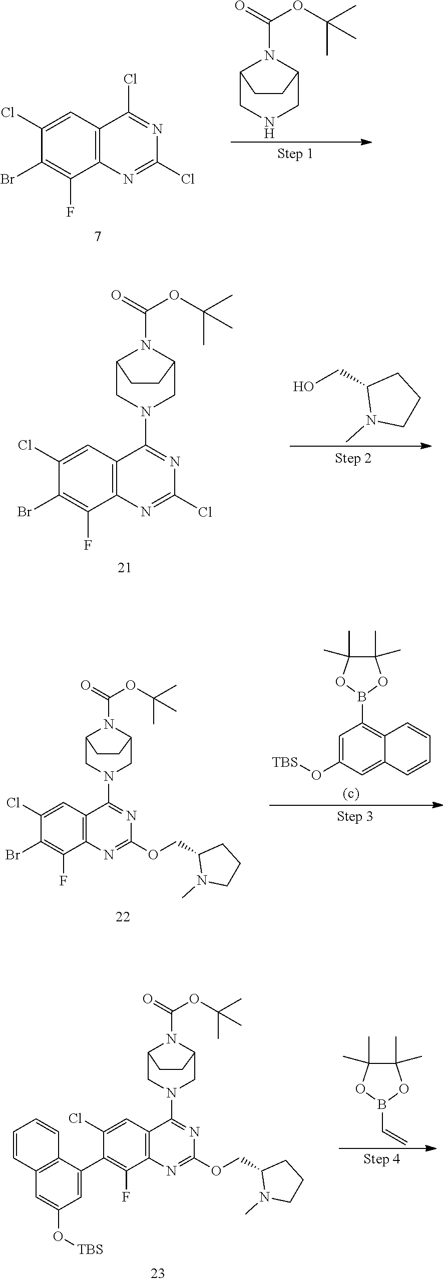

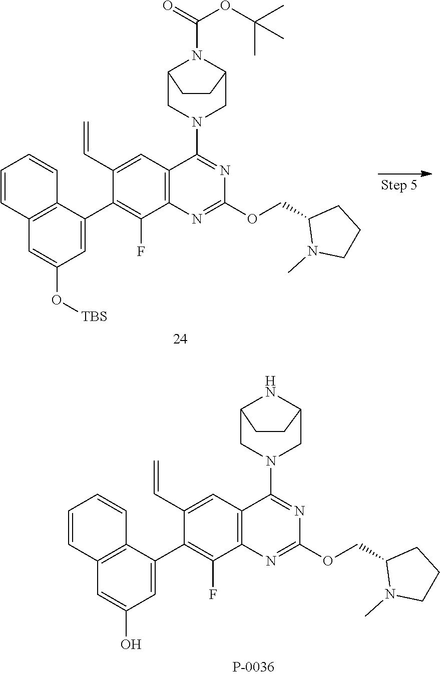

### Step 1: tert-Butyl 3-(7-bromo-2,6-dichloro-8-fluoro-quinazolin-4-yl)-3,8-diazabicyclo[3.2.1]octane-8-carboxylate (21)

To a solution of 7-bromo-2,4,6-trichloro-8-fluoro-quinazoline (7, 500 mg, 1.51 mmol), DIEA (587 mg, 4.54 mmol) in dioxane (5 mL), was added tert-butyl 3,8-diazabicyclo[3.2.1]octane-8-carboxylate (386 mg, 1.82 mmol) at room temperature, and then the reaction mixture was stirred at room temperature for 2 hrs. The reaction mixture was diluted with CH₂Cl₂ (10 mL) and washed with water and brine, dried over anhydrous Na₂SO₄, filtered and concentrated in vacuo. The residue was washed with hexane-ethyl acetate, and collected by filtration to give tert-butyl 3-(7-bromo-2,6-dichloro-8-fluoro-quinazolin-4-yl)-3,8-diazabicyclo[3.2.1]octane-8-carboxylate (21, 700 mg).

### Step 2: Tert-butyl 3-[7-bromo-6-chloro-8-fluoro-2-[[(2S)-1-methylpyrrolidin-2-yl]methoxy]quinazolin-4-yl]-3,8-diazabicyclo[3.2.1]octane-8-carboxylate (22)

To a solution of tert-butyl 3-(7-bromo-2,6-dichloro-8-fluoro-quinazolin-4-yl)-3,8-diazabicyclo[3.2.1]octane-8-carboxylate (21, 500 mg, 0.99 mmol), KF (115 mg, 1.98 mmol) in DMSO (4 mL), was added [(2S)-1-methylpyrrolidin-2-yl]methanol (341 mg, 2.96 mmol) at room temperature, and then the reaction mixture was stirred at 100° C. for 5 hours.

The reaction mixture was diluted with ethyl acetate (10 mL) and washed with water and brine, dried over anhydrous Na₂SO₄, filtered and concentrated in vacuo. The residue was purified by silica gel column chromatography (pre packed column, 24 g, MeOH/ethyl acetate=0 to 10%) to give tert-butyl 3-[7-bromo-6-chloro-8-fluoro-2-[[(2S)-1-methylpyrrolidin-2-yl]methoxy]quinazolin-4-yl]-3,8-diazabicyclo[3.2.1]octane-8-carboxylate (22, 300 mg). MS (ESI) [M+H⁺]⁺=586.21.

### Step 3: Tert-Butyl 3-[7-[3-[tert-butyl(dimethyl)silyl]oxy-1-naphthyl]-6-chloro-8-fluoro-2-[[(2S)-1-methylpyrrolidin-2-yl]methoxy]quinazolin-4-yl]-3,8-diazabicyclo[3.2.1]octane-8-carboxylate (23)

To a solution of tert-butyl 3-[7-bromo-6-chloro-8-fluoro-2-[[(2S)-1-methylpyrrolidin-2-yl]methoxy]quinazolin-4-yl]-3,8-diazabicyclo[3.2.1]octane-8-carboxylate (22, 85 mg, 0.145 mmol), tert-butyl-dimethyl-[[4-(4,4,5,5-tetramethyl-1,3,2-dioxaborolan-2-yl)-2-naphthyl]oxy]silane (3, 168 mg, 0.436 mmol) and Na₂CO₃ (46.2 mg, 0.436 mmol) in dioxane (2 mL) and H₂O (0.5 mL), was added Pd(PPh₃)₄ (16.8 mg, 0.015 mmol) at room temperature, and then the reaction mixture was stirred at 90° C. for 3 hrs. The reaction mixture was diluted with CH₂Cl₂ (10 mL) and washed with water and brine, dried over anhydrous Na₂SO₄, filtered and concentrated in vacuo. The residue was purified with silica gel column chromatography (MeOH/ethyl acetate=0 to 10%) to give tert-butyl 3-[7-[3-[tert-butyl(dimethyl)silyl]oxy-1-naphthyl]-6-chloro-8-fluoro-2-[[(2S)-1-methylpyrrolidin-2-yl]methoxy]quinazolin-4-yl]-3,8-diazabicyclo[3.2.1]octane-8-carboxylate (23, 100 mg). MS (ESI) [M+H]+=762.49.

### Step 4: Tert-butyl 3-[7-[3-[tert-butyl(dimethyl)silyl]oxy-1-naphthyl]-8-fluoro-2-[[(2S)-1-methylpyrrolidin-2-yl]methoxy]-6-vinyl-quinazolin-4-yl]-3,8-diazabicyclo[3.2.1]octane-8-carboxylate (24)

To a solution of tert-butyl 3-[7-[3-[tert-butyl(dimethyl)silyl]oxy-1-naphthyl]-6-chloro-8-fluoro-2-[[(2S)-1-methylpyrrolidin-2-yl]methoxy]quinazolin-4-yl]-3,8-diazabicyclo[3.2.1]octane-8-carboxylate (23, 56 mg, 0.073 mmol), 4,4,5,5-tetramethyl-2-vinyl-1,3,2-dioxaborolane (33.9 mg, 0.22 mmol) and Na₂CO₃ (23.4 mg, 0.22 mmol) in dioxane (2 mL) and H₂O (0.5 mL), was added SPhos Pd G4 (6.5 mg, 0.007 mmol) at room temperature and the reaction mixture was stirred at 90° C. for 1 hr. The reaction mixture was diluted with CH₂Cl₂ (10 mL), washed with water and brine, dried over anhydrous Na₂SO₄, filtered and concentrated in vacuo. The residue was purified with silica gel column chromatography (MeOH/ethyl acetate=0 to 10%) to give tert-butyl 3-[7-[3-[tert-butyl(dimethyl) silyl]oxy-1-naphthyl]-8-fluoro-2-[[(2S)-1-methylpyrrolidin-2-yl]methoxy]-6-vinyl-quinazolin-4-yl]-3,8-diazabicyclo[3.2.1]octane-8-carboxylate (24, 35 mg). MS (ESI) [M+H]+=754.6.

### Step 5: 4-[4-(3,8-Diazabicyclo[3.2.1]octan-3-yl)-8-fluoro-2-[[(2S)-1-methylpyrrolidin-2-yl]methoxy]-6-vinyl-quinazolin-7-yl]naphthalen-2-ol (P-0036)

To a solution of tert-butyl 3-[7-[3-[tert-butyl(dimethyl)silyl]oxy-1-naphthyl]-8-fluoro-2-[[(2S)-1-methylpyrrolidin-2-yl]methoxy]-6-vinyl-quinazolin-4-yl]-3,8-diazabicyclo[3.2.1]octane-8-carboxylate (24, 35 mg, 0.046 mmol) in MeOH (0.5 mL), was added 4M HCl in dioxane (1.5 mL) and the reaction was stirred at room temperature for 1 hr.

After the reaction was quenched with aqueous NaHCO₃, the reaction mixture was extracted with CH₂Cl₂-IPA (3:1, 10 mL). The extract was washed with brine, dried over anhydrous Na₂SO₄, and filtered and concentrated in vacuo. The residue was purified with reverse phase column chromatography (0.5% HCO₂H in CH₃CN/0.5% HCO₂H in H₂O=0 to 100%) to give 4-(3,8-diazabicyclo[3.2.1]octan-3-yl)-8-fluoro-2-[[(2S)-1-methylpyrrolidin-2-yl]methoxy]-6-vinyl-quinazolin-7-yl]naphthalen-2-ol (P-0036, 13 mg). MS (ESI) [M+H⁺]⁺=540.3.

### Example 13

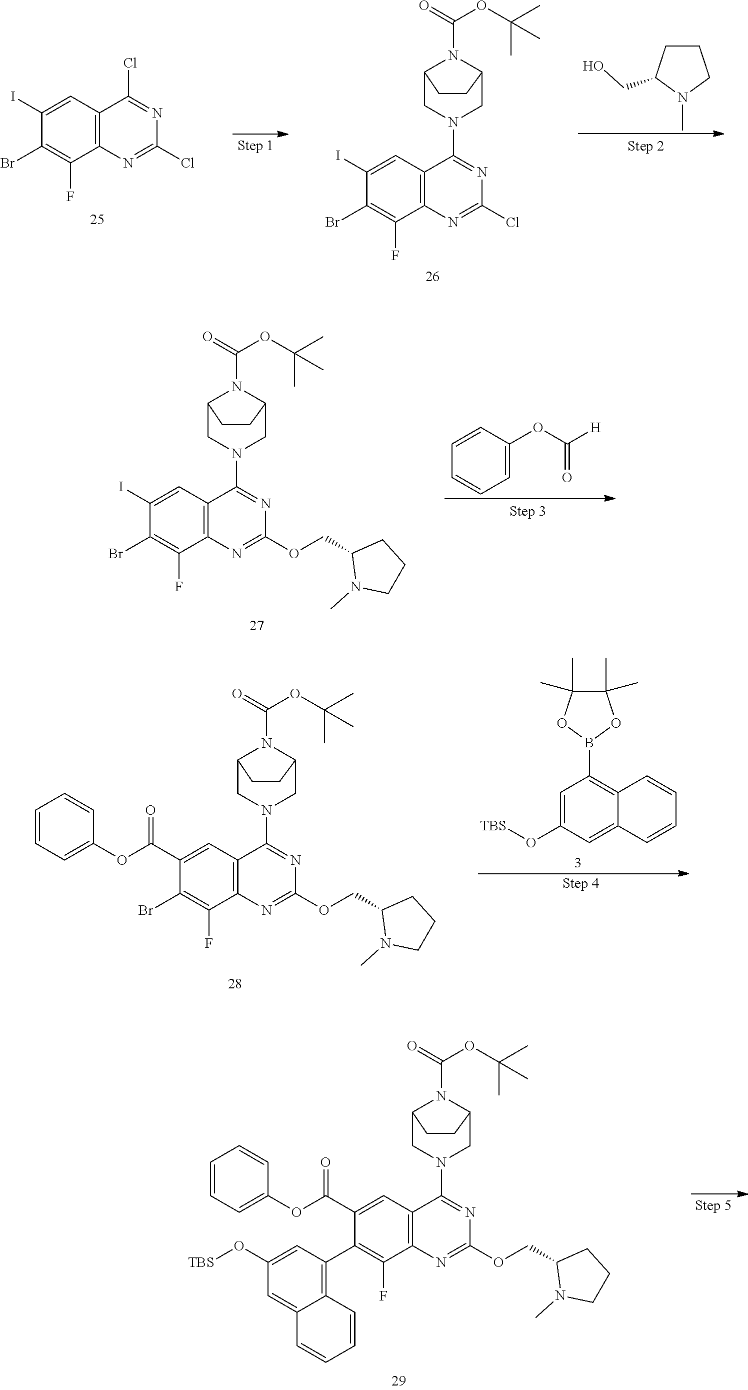

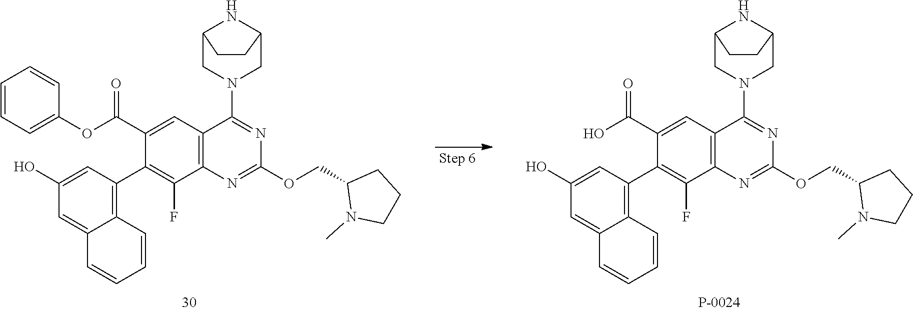 [SMILES: CN1CCC[C@H]1COc1nc(N2CC3CCC(C2)N3)c2cc(C(=O)Oc3ccccc3)c(-c3cc(O)cc4ccccc34)c(F)c2n1 ; CN1CCC[C@H]1COc1nc(N2CC3CCC(C2)N3)c2cc(C(=O)O)c(-c3cc(O)cc4ccccc34)c(F)c2n1]

### Step 1: tert-Butyl 3-(7-bromo-2-chloro-8-fluoro-6-iodo-quinazolin-4-yl)-3,8-diazabicyclo[3.2.1]octane-8-carboxylate (26)

To a solution of 7-bromo-2,4-dichloro-8-fluoro-6-iodo-quinazoline (25, 880 mg, 2.09 mmol), DIEA (809 mg, 6.26 mmol) in dioxane (10 mL), was added tert-butyl 3,8-diazabicyclo[3.2.1]octane-8-carboxylate (487 mg, 2.30 mmol) at room temperature, and then the reaction mixture was stirred at room temperature for 2 hrs. The reaction mixture was diluted with CH₂Cl₂ (10 mL) and washed with water and brine, dried over anhydrous Na₂SO₄, filtered and concentrated in vacuo. The residue was washed with hexane:ethyl acetate (9:1) and collected by filtration to give tert-butyl 3-(7-bromo-2-chloro-8-fluoro-6-iodo-quinazolin-4-yl)-3,8-diazabicyclo[3.2.1]octane-8-carboxylate (26, 1,180 mg).

### Step 2: tert-butyl 3-[7-bromo-8-fluoro-6-iodo-2-[[(2S)-1-methylpyrrolidin-2-yl]methoxy]quinazolin-4-yl]-3,8-diazabicyclo[3.2.1]octane-8-carboxylate (27)

To a solution of tert-butyl 3-(7-bromo-2-chloro-8-fluoro-6-iodo-quinazolin-4-yl)-3,8-diazabicyclo[3.2.1]octane-8-carboxylate (26, 1180 mg, 1.97 mmol), KF (229 mg, 3.95 mmol) in DMSO (10 mL), was added [(2S)-1-methylpyrrolidin-2-yl]methanol (1140 mg, 9.87 mmol) at room temperature, and then the reaction mixture was stirred at 120° C. for 5 hours. The reaction mixture was diluted with ethyl acetate (10 mL) and washed with water and brine, dried over anhydrous Na₂SO₄, filtered and concentrated in vacuo. The residue was purified by silica gel column chromatography (MeOH/CH₂Cl₂=0 to 10%) to give tert-butyl 3-[7-bromo-8-fluoro-6-iodo-2-[[(2S)-1-methylpyrrolidin-2-yl]methoxy]quinazolin-4-yl]-3,8-diazabicyclo[3.2.1]octane-8-carboxylate (25, 600 mg). MS (ESI) [M+H⁺]⁺=678.06.

### Step 3: Phenyl 7-bromo-4-(8-tert-butoxycarbonyl-3,8-diazabicyclo[3.2.1]octan-3-yl)-8-fluoro-2-[[(2S)-1-methylpyrrolidin-2-yl]methoxy]quinazoline-6-carboxylate (28)

To a solution of tert-butyl 3-[7-bromo-8-fluoro-6-iodo-2-[[(2S)-1-methylpyrrolidin-2-yl]methoxy]quinazolin-4-yl]-3,8-diazabicyclo[3.2.1]octane-8-carboxylate (27, 50 mg, 0.074 mmol), phenyl formate (27.1 mg, 0.222 mmol), DBU (33.8 mg, 0.222 mmol) and Xantphos (42.8 mg, 0.074 mmol) in CH₃CN (1 mL), was added Pd(OAc)₂ (8.3 mg, 0.037 mmol) at room temperature, and the reaction mixture was stirred at 90° C. for 2 hrs. The reaction mixture was diluted with CH₂Cl₂ (10 mL) and washed with water and brine, dried over anhydrous Na₂SO₄, filtered and concentrated in vacuo. The residue was purified by silica gel column chromatography (MeOH/CH₂Cl₂=0 to 20%) to give phenyl 7-bromo-4-(8-tert-butoxycarbonyl-3,8-diazabicyclo[3.2.1]octan-3-yl)-8-fluoro-2-[[(2S)-1-methylpyrrolidin-2-yl]methoxy]quinazoline-6-carboxylate (28, 25 mg). MS (ESI) [M+H⁺]⁺=670.2.

### Step 4: Phenyl 4-(8-tert-butoxycarbonyl-3,8-diazabicyclo[3.2.1]octan-3-yl)-7-[3-[tert-butyl(dimethyl)silyl]oxy-1-naphthyl]-8-fluoro-2-[[(2S)-1-methylpyrrolidin-2-yl]methoxy]quinazoline-6-carboxylate (29)

To a solution of phenyl 7-bromo-4-(8-tert-butoxycarbonyl-3,8-diazabicyclo[3.2.1]octan-3-yl)-8-fluoro-2-[[(2S)-1-methylpyrrolidin-2-yl]methoxy]quinazoline-6-carboxylate (28, 17 mg, 0.025 mmol), tert-butyl-dimethyl-[[4-(4,4,5,5-tetramethyl-1,3,2-dioxaborolan-2-yl)-2-naphthyl]oxy]silane (3, 29.2 mg, 0.076 mmol) and Na₂CO₃ (8.1 mg, 0.076 mmol) in dioxane (2 mL) and H₂O (0.5 mL), was added Pd(PPh₃)₄ (2.9 mg, 0.003 mmol) at room temperature and the reaction mixture was stirred at 90° C. for 1 hr. The reaction mixture was diluted with CH₂Cl₂ (10 mL) and washed with water and brine, dried over anhydrous Na₂SO₄, filtered and concentrated in vacuo. The residue was purified by silica gel column chromatography (MeOH/CH₂Cl₂=0 to 10%) to provide phenyl 4-(8-tert-butoxycarbonyl-3,8-diazabicyclo[3.2.1]octan-3-yl)-7-[3-[tert-butyl(dimethyl)silyl]oxy-1-naphthyl]-8-fluoro-2-[[(2S)-1-methylpyrrolidin-2-yl]methoxy]quinazoline-6-carboxylate (29, 21 mg).

### Step 5: Phenyl 4-(3,8-diazabicyclo[3.2.1]octan-3-yl)-8-fluoro-7-(3-hydroxy-1-naphthyl)-2-[[(2S)-1-methylpyrrolidin-2-yl]methoxy]quinazoline-6-carboxylate (30)

To a solution of phenyl 4-(8-tert-butoxy carbonyl-3,8-diazabicyclo[3.2.1]octan-3-yl)-7-[3-[tert-butyl(dimethyl)silyl]oxy-1-naphthyl]-8-fluoro-2-[[(2S)-1-methylpyrrolidin-2-yl]methoxy]quinazoline-6-carboxylate (29, 18 mg, 0.021 mmol) in MeOH (0.5 mL), was added 4M HCl in dioxane (1.5 mL). The reaction mixture was stirred at room temperature for 1 hr.

The reaction mixture was concentrated down to give phenyl 4-(3,8-diazabicyclo[3.2.1]octan-3-yl)-8-fluoro-7-(3-hydroxy-1-naphthyl)-2-[[(2S)-1-methylpyrrolidin-2-yl]methoxy]quinazoline-6-carboxylate (30, 13 mg).

### Step 6: 4-(3,8-Diazabicyclo[3.2.1]octan-3-yl)-8-fluoro-7-(3-hydroxy-1-naphthyl)-2-[[(2S)-1-methylpyrrolidin-2-yl]methoxy]quinazoline-6-carboxylic acid (P-0024)

To a solution of phenyl 4-(3,8-diazabicyclo[3.2.1]octan-3-yl)-8-fluoro-7-(3-hydroxy-1-naphthyl)-2-[[(2S)-1-methylpyrrolidin-2-yl]methoxy]quinazoline-6-carboxylate (30, 13.5 mg, 0.021 mmol) in MeOH (1 mL), was added 1M NaOH (0.5 mL) at room temperature, and the reaction mixture was stirred at 60° C. for 1 hour. The reaction mixture was allowed to room temperature, and then the reaction mixture was neutralized with 1M HCl. The reaction mixture was concentrated in vacuo, and the residue was purified with reverse phase column chromatography (0.5% HCO₂H in CH₃CN/0.5% HCO₂H in H₂O=0 to 100%) to give 4-(3,8-diazabicyclo[3.2.1]octan-3-yl)-8-fluoro-7-(3-hydroxy-1-naphthyl)-2-[[(2S)-1-methylpyrrolidin-2-yl]methoxy]quinazoline-6-carboxylic acid (P-0024, 9 mg). MS (ESI) [M+H⁺]⁺=558.3.

### Example 14

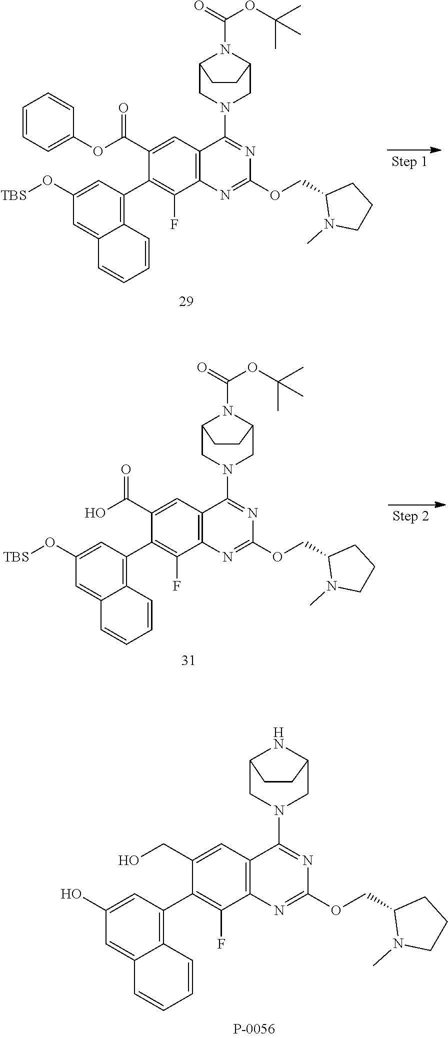

### Step 1: Tert-butyl 3-[7-[3-[tert-butyl(dimethyl)silyl]oxy-1-naphthyl]-8-fluoro-6-(hydroxyl methyl)-2-[[(2S)-1-methylpyrrolidin-2-yl]methoxy]quinazolin-4-yl]-3,8-diazabicyclo[3.2.1]octane-8-carboxylate (31)

To a solution of phenyl 4-(8-tert-butoxy carbonyl-3,8-diazabicyclo[3.2.1]octan-3-yl)-7-[3-[tert-butyl(dimethyl)silyl]oxy-1-naphthyl]-8-fluoro-2-[[(2S)-1-methylpyrrolidin-2-yl]methoxy]quinazoline-6-carboxylate (29, 20 mg, 0.024 mmol) in THF (2 mL), was added slowly LAH (2 M in THF, 0.24 mL, 0.047 mmol) at 0° C. and the reaction mixture was stirred at 0° C. for 1 hour.

After the reaction was quenched with 1N NaOH solution, the reaction mixture was diluted with CH₂Cl₂ (10 mL) and washed with water and brine, dried over anhydrous Na₂SO₄, filtered and concentrated in vacuo to afford tert-butyl 3-[7-[3-[tert-butyl(dimethyl)silyl]oxy-1-naphthyl]-8-fluoro-6-(hydroxymethyl)-2-[[(2S)-1-methylpyrrolidin-2-yl]methoxy]quinazolin-4-yl]-3,8-diazabicyclo[3.2.1]octane-8-carboxylate (31, 17 mg).

### Step 2: 4-[4-(3,8-diazabicyclo[3.2.1]octan-3-yl)-8-fluoro-6-(hydroxymethyl)-2-[[(2S)-1-methylpyrrolidin-2-yl]methoxy]quinazolin-7-yl]naphthalen-2-ol (P-0056)

To a solution of tert-butyl 3-[7-[3-[tert-butyl(dimethyl)silyl]oxy-1-naphthyl]-8-fluoro-6-(hydroxymethyl)-2-[[(2S)-1-methylpyrrolidin-2-yl]methoxy]quinazolin-4-yl]-3,8-diazabicyclo[3.2.1]octane-8-carboxylate (31, 17 mg, 0.022 mmol) in MeOH (0.5 mL), was added 4M HCl in dioxane (1.5 mL) and the reaction was stirred at room temperature for 1 hr.

After the reaction was quenched with aqueous NaHCO₃, the reaction mixture was extracted with CH₂Cl₂-IPA (3:1, 10 mL). The extract was washed with brine (5 mL), dried over anhydrous Na₂SO₄, filtered and concentrated in vacuo. The residue was purified by reverse phase column chromatography (0.5% HCO₂H in CH₃CN/0.5% HCO₂H in H₂O=0 to 100%) to give 4-[4-(3,8-diazabicyclo[3.2.1]octan-3-yl)-8-fluoro-6-(hydroxymethyl)-2-[[(2S)-1-methylpyrrolidin-2-yl]methoxy]quinazolin-7-yl]naphthalen-2-ol (P-0056, 7 mg). MS (ESI) [M+H⁺]⁺=544.3.

### Intermediate 33

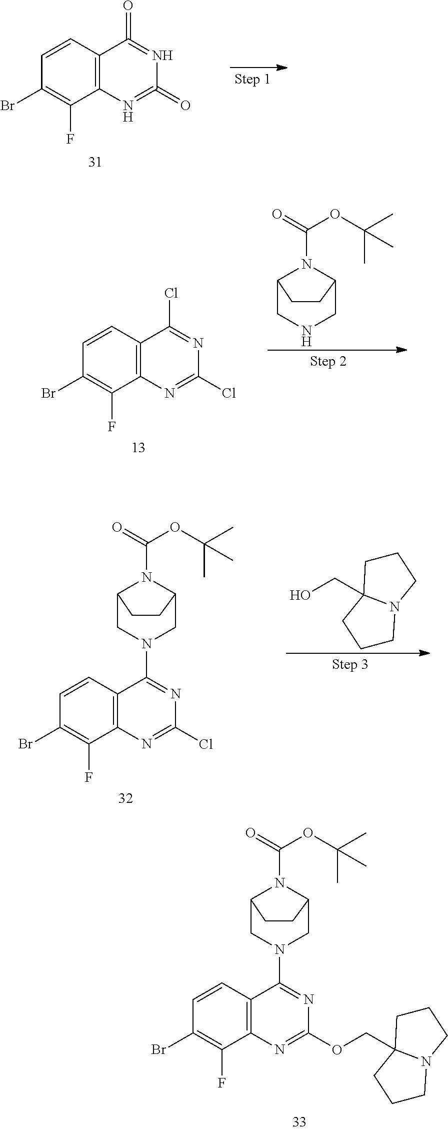

### Step 1: 7-bromo-2,4-dichloro-8-fluoroquinazoline (13)

To a stirred solution of 7-bromo-8-fluoroquinazoline-2,4(1H,3H)-dione (31, 780 mg, 3.01 mmol) in POCl₃ (14 mL), diisopropylethylamine (1.61 mL, 9.03 mmol) was added at room temperature. The reaction mixture was then stirred at 150° C. for 3 hours. Upon completion, the reaction mixture was cooled to room temperature, poured onto crushed ice, extracted with 20 ml ethyl acetate, and washed with water and brine. The organic layer was dried over anhydrous Na₂SO₄, filtered, and concentrated in vacuo. The residue was purified by silica gel column chromatography (AcOEt/Hexane=0 to 10%) to give 7-bromo-2,4-dichloro-8-fluoroquinazoline (13, 580 mg).

### Step 2: Tert-butyl 3-(7-bromo-2-chloro-8-fluoro-quinazolin-4-yl)-3,8-diazabicyclo[3.2.1]octane-8-carboxylate (32)

To a solution of 7-bromo-2,4-dichloro-8-fluoroquinazoline (13, 300 mg, 1.01 mmol) in dioxane (3 mL), DIEA (0.54 mL, 3.04 mmol) was added, followed by tert-butyl (1R,5S)-3,8-diazabicyclo[3.2.1]octane-8-carboxylate (237 mg, 1.16 mmol). The reaction mixture was stirred at room temperature for 2 hrs. After LC-MS showed completion, the reaction mixture was diluted with CH₂Cl₂ (10 mL) and washed twice with water and brine, dried over anhydrous Na₂SO₄, filtered and concentrated in vacuo. The residue was purified by silica gel column chromatography with Hexane:EtOAc (9:1) to provide tert-butyl (1R,5S)-3-(7-bromo-2-chloro-8-fluoroquinazolin-4-yl)-3,8-diazabicyclo[3.2.1]octane-8-carboxylate (32, 450 mg).

### Step 3: Tert-butyl 3-[7-bromo-8-fluoro-2-(1,2,3,5,6,7-hexahydropyrrolizin-8-ylmethoxy)quinazolin-4-yl]-3,8-diazabicyclo[3.2.1]octane-8-carboxylate (33)

To a solution of tert-butyl (1R,5S)-3-(7-bromo-2-chloro-8-fluoroquinazolin-4-yl)-3,8-diazabicyclo[3.2.1]octane-8-carboxylate (32, 200 mg, 0.424 mmol) in DMSO (4 mL), KF (49 mg, 0.848 mmol) was added at room temperature, followed by (tetrahydro-1H-pyrrolizin-7a(5H)-yl)methanol (299 mg, 2.12 mmol). The reaction mixture was stirred at 120° C. for 5 hours. The reaction mixture was diluted with EtOAc and washed with water and brine. The organic phase was dried over anhydrous Na₂SO₄, filtered, and concentrated in vacuo. The residue was purified by silica gel column chromatography (MeOH/CH₂Cl₂=0 to 20%) to give tert-butyl (1R,5S)-3-(7-bromo-8-fluoro-2-((tetrahydro-1H-pyrrolizin-7a(5H)-yl)methoxy)quinazolin-4-yl)-3,8-diazabicyclo[3.2.1]octane-8-carboxylate (32, 130 mg).

### Example 15

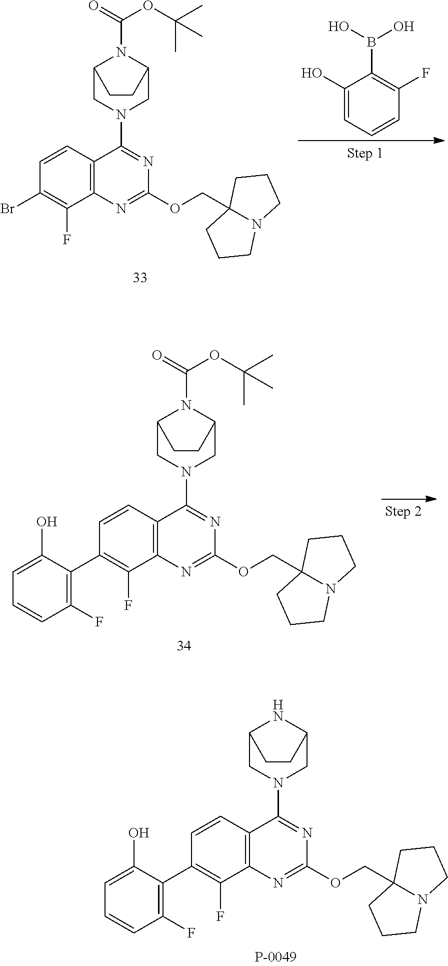 [SMILES: CC(C)(C)OC(=O)N1C2CCC1CN(c1nc(OCC34CCCN3CCC4)nc3c(F)c(Br)ccc13)C2 ; OB(O)c1c(O)cccc1F ; CC(C)(C)OC(=O)N1C2CCC1CN(c1nc(OCC34CCCN3CCC4)nc3c(F)c(-c4c(O)cccc4F)ccc13)C2 ; Oc1cccc(F)c1-c1ccc2c(N3CC4CCC(C3)N4)nc(OCC34CCCN3CCC4)nc2c1F]

### Step 1: (1R,5S)-3-(8-fluoro-7-(2-fluoro-6-hydroxyphenyl)-2-((tetrahydro-1H-pyrrolizin-7a(5H)-yl)methoxy)quinazolin-4-yl)-3,8-diazabicyclo[3.2.1]octane-8-carboxylate (34)

To a solution of compound tert-butyl (1R,5S)-3-(7-bromo-8-fluoro-2-((tetrahydro-1H-pyrrolizin-7a(5H)-yl)methoxy)quinazolin-4-yl)-3,8-diazabicyclo[3.2.1]octane-8-carboxylate (33, 280 mg, 0.139 mmol) and (2-fluoro-6-hydroxyphenyl)boronic acid (64.9 mg, 0.416 mmol) in 2 mL dioxane, Na₂CO₃ (73.5 mg, 0.694 mmol) and H₂O (0.5 mL) was added, followed by RuPhos Pd Gen4 (11.8 mg, 0.014 mmol). The reaction mixture was stirred at 90° C. for 2 hours. After LCMS showed completion, the reaction mixture was cooled down to room temperature and diluted with CH₂C₁₋₂ (10 mL). Then the reaction mixture was washed with water and brine, dried over anhydrous Na₂SO₄, filtered, and concentrated in vacuo. The residue was purified by silica gel column chromatography (MeOH/CH₂C₁₋₂=0 to 80%) to give tert-butyl (1R,5S)-3-(8-fluoro-7-(2-fluoro-6-hydroxyphenyl)-2-((tetrahydro-1H-pyrrolizin-7a(5H)-yl)methoxy)quinazolin-4-yl)-3,8-diazabicyclo[3.2.1]octane-8-carboxylate (34, 50 mg, 59.2%).

### Step 2: 2-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)-3-fluorophenol (P-0049)

To a solution of tert-butyl (1R,5S)-3-(8-fluoro-7-(2-fluoro-6-hydroxyphenyl)-2-((tetrahydro-1H-pyrrolizin-7a(5H)-yl)methoxy)quinazolin-4-yl)-3,8-diazabicyclo[3.2.1]octane-8-carboxylate (34, 35 mg, 0.082 mmol) in MeOH (0.5 mL), 4M HCl in dioxane (1.5 mL) was added and the reaction was stirred at room temperature for 1 hr. After the reaction was quenched with saturated NaHCO₃ solution, the reaction mixture was extracted with CH₂C₁₋₂:IPA (3:1, 10 mL). The extract was washed with brine (5 mL), dried over anhydrous Na₂SO₄, filtered and concentrated in vacuo. The residue was purified by reverse phase column chromatography (0.5% HCO₂H in CH₃CN/0.5% HCO₂H in H₂O=0 to 100%) to give 2-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)-3-fluorophenol (P-0049 15 mg). MS (ESI) [M+H⁺]⁺=508.2.

### Example 16

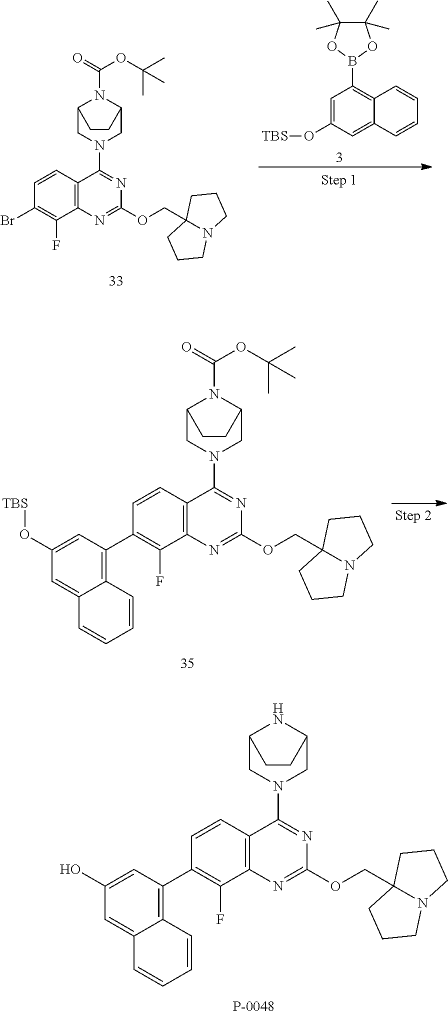

### Step 1: Tert-butyl 3-[7-[3-[tert-butyl(dimethyl)silyl]oxy-1-naphthyl]-8-fluoro-2-[[(2S)-1-methylpyrrolidin-2-yl]methoxy]pyrido[4,3-d]pyrimidin-4-yl]-3,8-diazabicyclo[3.2.1]octane-8-carboxylate (35)

To a solution of tert-butyl 3-[7-bromo-8-fluoro-2-(1,2,3,5,6,7-hexahydropyrrolizin-8-ylmethoxy)quinazolin-4-yl]-3,8-diazabicyclo[3.2.1]octane-8-carboxylate (33, 50 mg, 0.087 mmol) and tert-butyldimethyl((4-(4,4,5,5-tetramethyl-1,3,2-dioxaborolan-2-yl)naphthalen-2-yl)oxy)silane (3, 100 mg, 0.26 mmol) in 2 mL dioxane, Na₂CO₃ (27.6 mg, 0.26 mmol) and H₂O (0.5 mL) was added, followed by Pd(PPh₃)₄ (10 mg, 0.009 mmol). The reaction mixture was stirred at 90° C. for 2 hours. After LCMS showed completion, the reaction mixture was cooled down to room temperature and diluted with CH₂Cl₂ (10 mL). Then the reaction mixture was washed with water and brine, dried over anhydrous Na₂SO₄, filtered and concentrated in vacuo. The residue was purified by silica gel column chromatography (MeOH/CH₂Cl₂=0 to 50%) to provide tert-butyl 3-[7-[3-[tert-butyl(dimethyl)silyl]oxy-1-naphthyl]-8-fluoro-2-[[(2S)-1-methylpyrrolidin-2-yl]methoxy]pyrido[4,3-d]pyrimidin-4-yl]-3,8-diazabicyclo[3.2.1]octane-8-carboxylatt (35, 50 mg).

### Step 2: 4-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)naphthalen-2-ol (P-0048)

To a solution of tert-butyl 3-[7-[3-[tert-butyl(dimethyl)silyl]oxy-1-naphthyl]-8-fluoro-2-[[(2S)-1-methylpyrrolidin-2-yl]methoxy]pyrido[4,3-d]pyrimidin-4-yl]-3,8-diazabicyclo[3.2.1]octane-8-carboxylatt (35, 98 mg, 0.134 mmol) in MeOH (0.5 mL), 4M HCl in dioxane (1.5 mL) was added and the reaction was stirred at room temperature for 1 hr. After the reaction was quenched with saturated NaHCO₃ solution, the reaction mixture was extracted with CH₂Cl₂:IPA (3:1, 10 mL). The extract was washed with brine, dried over anhydrous Na₂SO₄, filtered, and concentrated in vacuo. The residue was purified by reverse phase column chromatography (0.5% HCO₂H in CH₃CN/0.5% HCO₂H in H₂O=0 to 100%) to provide 4-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)naphthalen-2-ol (P-0048, 25 mg, 69.8% yield). MS (ESI) [M+H⁺]⁺=540.2.

### Example 16a

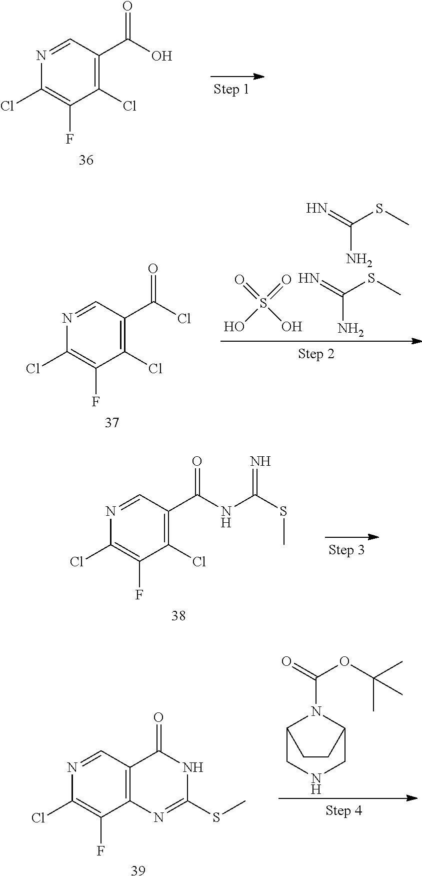

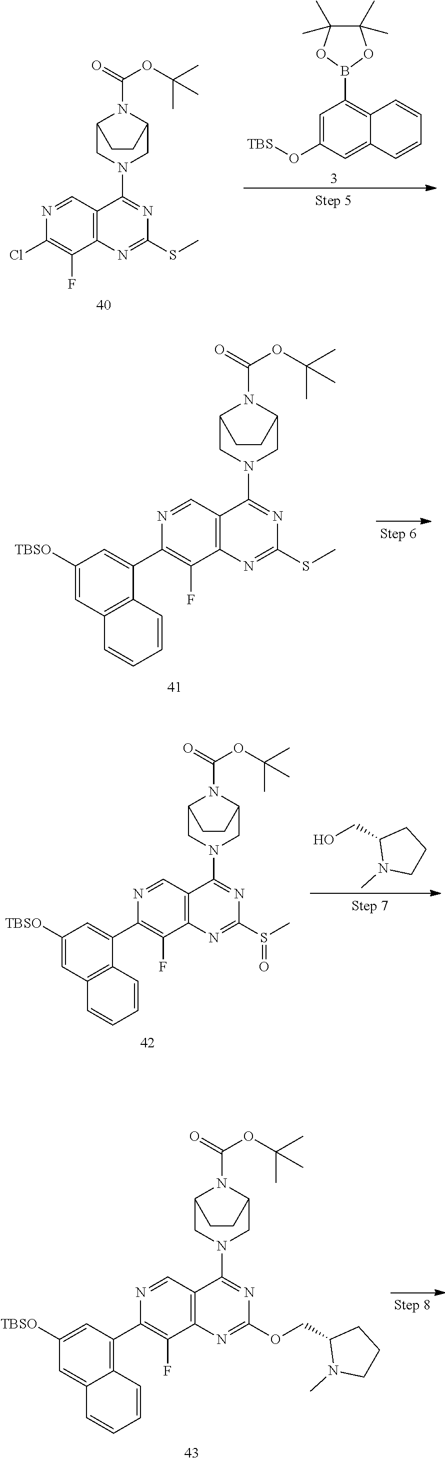

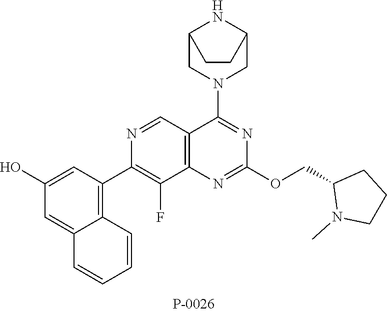 [SMILES: CN1CCC[C@H]1COc1nc(N2CC3CCC(C2)N3)c2cnc(-c3cc(O)cc4ccccc34)c(F)c2n1]

### Step 1: 4,6-dichloro-5-fluoro-pyridine-3-carbonyl chloride (37)

To a solution of 4,6-dichloro-5-fluoronicotinic acid (36, 1 g, 4.67 mmol) in CH₂Cl₂ (20 mL), oxalyl chloride (0.45 mL, 5.24 mmol) was added slowly and then DMF (35 mg, 0.476 mmol) was added at room temperature. The reaction mixture was stirred at 50° C. for 2 hours. The reaction mixture was concentrated in vacuo and then azeotroped with toluene to give 4,6-dichloro-5-fluoronicotinoyl chloride (37, 1.08 g).

### Step 2: 4,6-dichloro-5-fluoro-N-(methylsulfanylcarbonimidoyl)pyridine-3-carboxamide (38)

To a solution of 2-methylisothiourea sulfuric acid (3.29 g, 11.8 mmol) in 1M NaOH aq. (15 ml), a solution of 4,6-dichloro-5-fluoronicotinoyl chloride (37, 1.08 g, 4.73 mmol) in diethyl ether (4 mL) was slowly added at 0° C., and the reaction mixture was stirred for 1 hour at 0° C. The solid precipitate were collected by filtration and dried to give methyl(4,6-dichloro-5-fluoronicotinoyl)carbamimidothioate (38, 520 mg).

### Step 3: 7-chloro-8-fluoro-2-methylsulfanyl-3H-pyrido[4,3-d]pyrimidin-4-one (39)

Methyl(4,6-dichloro-5-fluoronicotinoyl)carbamimidothioate (38, 520 mg, 1.84 mmol) was dissolved in DMF (4 mL) and the reaction mixture was stirred at 120° C. for 2 hours. The reaction mixture was cooled to room temperature and water (20 mL) was added. The solid precipitate was filtered and dried under vacuo to give 7-chloro-8-fluoro-2(methylthio)pyrido[4,3-d]pyrimidin-4(3H)-one (39, 320 mg, 70.6% yield).

### Step 4: Tert-butyl 3-(7-chloro-8-fluoro-2-methylsulfanyl-pyrido[4,3-d]pyrimidin-4-yl)-3,8-diazabicyclo[3.2.1]octane-8-carboxylate (40)

To a solution of 7-chloro-8-fluoro-2-(methylthio)pyrido[4,3-d]pyrimidin-4(3H)-one (39, 160 mg, 0.651 mmol) in 5 mL CH₂Cl₂, was added DIEA (421 mg, 3.26 mmol). Then Tf₂O (0.16 mL, 0.977 mmol) was added dropwise at 0° C. The reaction mixture was stirred at 0° C. for 30 min, followed by the addition of tert-butyl (1R,5S)-3,8-diazabicyclo[3.2.1]octane-8-carboxylate. The reaction mixture was warmed up to room temperature and stirred for 30 min. The reaction mixture was diluted with CH₂Cl₂ (10 mL) and washed with water and brine, dried over anhydrous Na₂SO₄, filtered, and concentrated in vacuo. The residue was purified by silica gel column chromatography (EtOAc/Hexane=0 to 30%) to give tert-butyl (1R,5S)-3-(7-chloro-8-fluoro-2-(methylthio)pyrido[4,3-d]pyrimidin-4-yl)-3,8-diazabicyclo[3.2.1]octane-8-carboxylate (40, 210 mg).

### Step 5: Tert-butyl 3-[7-[3-[tert-butyl(dimethyl)silyl]oxy-1-naphthyl]-8-fluoro-2-methylsulfanyl-pyrido[4,3-d]pyrimidin-4-yl]-3,8-diazabicyclo[3.2.1]octane-8-carboxylate (41)

To a solution of tert-butyl (1R,5S)-3-(7-chloro-8-fluoro-2-(methylthio)pyrido[4,3-d]pyrimidin-4-yl)-3,8-diazabicyclo[3.2.1]octane-8-carboxylate (40, 210 mg, 0.477 mmol) and tert-butyldimethyl((4-(4,4,5,5-tetramethyl-1,3,2-dioxaborolan-2-yl)naphthalen-2-yl)oxy)silane (3, 367 mg, 0.955 mmol) in 4 mL dioxane, Na₂CO₃ (152 mg, 1.43 mmol) and H₂O (1 mL) was added, followed by Pd(PPh₃)₄ (55 mg, 0.048 mmol). The reaction mixture was stirred at 90° C. for 2 hrs. After LCMS showed completion, the reaction mixture was cooled down to room temperature and diluted with CH₂Cl₂ (10 mL). Then the reaction mixture was washed with water and brine, dried over anhydrous Na₂SO₄, filtered, and concentrated in vacuo. The residue was purified by silica gel column chromatography (EtOAc/Hexane=0 to 30%) to give tert-butyl (1R,5S)-3-(7-(3-((tert-butyldimethylsilyl)oxy)naphthalen-1-yl)-8-fluoro-2-(methylthio)pyrido[4,3-d]pyrimidin-4-yl)-3,8-diazabicyclo[3.2.1]octane-8-carboxylate (41, 283 mg).

### Step 6: Tert-butyl 3-[7-[3-[tert-butyl(dimethyl)silyl]oxy-1-naphthyl]-8-fluoro-2-methylsulfinyl-pyrido[4,3-d]pyrimidin-4-yl]-3,8-diazabicyclo[3.2.1]octane-8-carboxylate (42)

To a solution of tert-butyl (1R,5S)-3-(7-(3-((tert-butyldimethylsilyl)oxy)naphthalen-1-yl)-8-fluoro-2-(methylthio)pyrido[4,3-d]pyrimidin-4-yl)-3,8-diazabicyclo[3.2.1]octane-8-carboxylate (41, 90 mg, 0.136 mmol) in CH₂Cl₂ (2 mL), mCPBA (33.5 mg, 0.150 mmol, 77%) was added at 0° C. and then the reaction mixture was stirred for 30 min at 0° C. The reaction was quenched with saturated Na₂S₂O₃ aq. (5 mL) and then the reaction mixture was diluted with CH₂Cl₂ (10 mL) and concentrated in vacuo to give tert-butyl (1R,5S)-3-(7-(3-((tert-butyldimethylsilyl)oxy)naphthalen-1-yl)-8-fluoro-2-(methylsulfinyl)pyrido[4,3-d]pyrimidin-4-yl)-3,8-diazabicyclo[3.2.1]octane-8-carboxylate (42, 92 mg).

### Step 7: Tert-butyl 3-[7-[3-[tert-butyl(dimethyl)silyl]oxy-1-naphthyl]-8-fluoro-2-[[(2S)-1-methylpyrrolidin-2-yl]methoxy]pyrido[4,3-d]pyrimidin-4-yl]-3,8-diazabicyclo[3.2.1]octane-8-carboxylate (43)

To a solution of tert-butyl (1R,5S)-3-(7-(3-((tert-butyldimethylsilyl)oxy)naphthalen-1-yl)-8-fluoro-2-(methylsulfinyl)pyrido[4,3-d]pyrimidin-4-yl)-3,8-diazabicyclo[3.2.1]octane-8-carboxylate (42, 92 mg, 0.136 mmol) and (S)-(1-methylpyrrolidin-2-yl)methanol (31.3 mg, 0.271 mmol) in THF (2 mL), sodium hydride (11 mg, 60% in mineral oil, 0.271 mmol) was added at 0° C. and the reaction mixture was stirred for 30 min at 0° C. Upon completion, the reaction mixture was quenched with saturated NH₄Cl aq. (2 mL), and the reaction mixture was extracted with EtOAc. The extract was washed with brine, dried over anhydrous Na₂SO₄, filtered and concentrated in vacuo to give tert-butyl (1R,5S)-3-(7-(3-((tert-butyldimethylsilyl)oxy)naphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-8-carboxylate (43, 98 mg).

### Step 8: 4-(4-((1R,5S)-3,8-diazabicyclo[3.2.1]octan-3-yl)-8-fluoro-2-(((S)-1-methylpyrrolidin-2-yl)methoxy)pyrido[4,3-d]pyrimidin-7-yl)naphthalen-2-ol (P-0026)

To a solution of tert-butyl (1R,5S)-3-(7-(3-((tert-butyldimethylsilyl)oxy)naphthalen-1-yl)-8-fluoro-2-(((S)-1-methylpyrrolidin-2-yl)methoxy)pyrido[4,3-d]pyrimidin-4-yl)-3,8-diazabicyclo[3.2.1]octane-8-carboxylate (43, 98 mg, 0.134 mmol) in MeOH (0.5 mL), 4M HCl in dioxane (1.5 mL) was added, and the reaction was stirred at room temperature for 1 hr. After the reaction was quenched with saturated NaHCO₃ solution, the reaction mixture was extracted with CH₂Cl₂:IPA (3:1, 10 mL). The extract was washed with brine, dried over anhydrous Na₂SO₄, filtered, and concentrated in vacuo. The residue was purified by reverse phase column chromatography (0.5% HCO₂H in CH₃CN/0.5% HCO₂H in H₂O=0 to 100%) to provide 4-(4-((1R,5S)-3,8-diazabicyclo[3.2.1]octan-3-yl)-8-fluoro-2-(((S)-1-methylpyrrolidin-2-yl)methoxy)pyrido[4,3-d]pyrimidin-7-yl)naphthalen-2-ol (P-0026, 10 mg). MS (ESI) [M+H⁺]⁺=515.3.

### Example 17

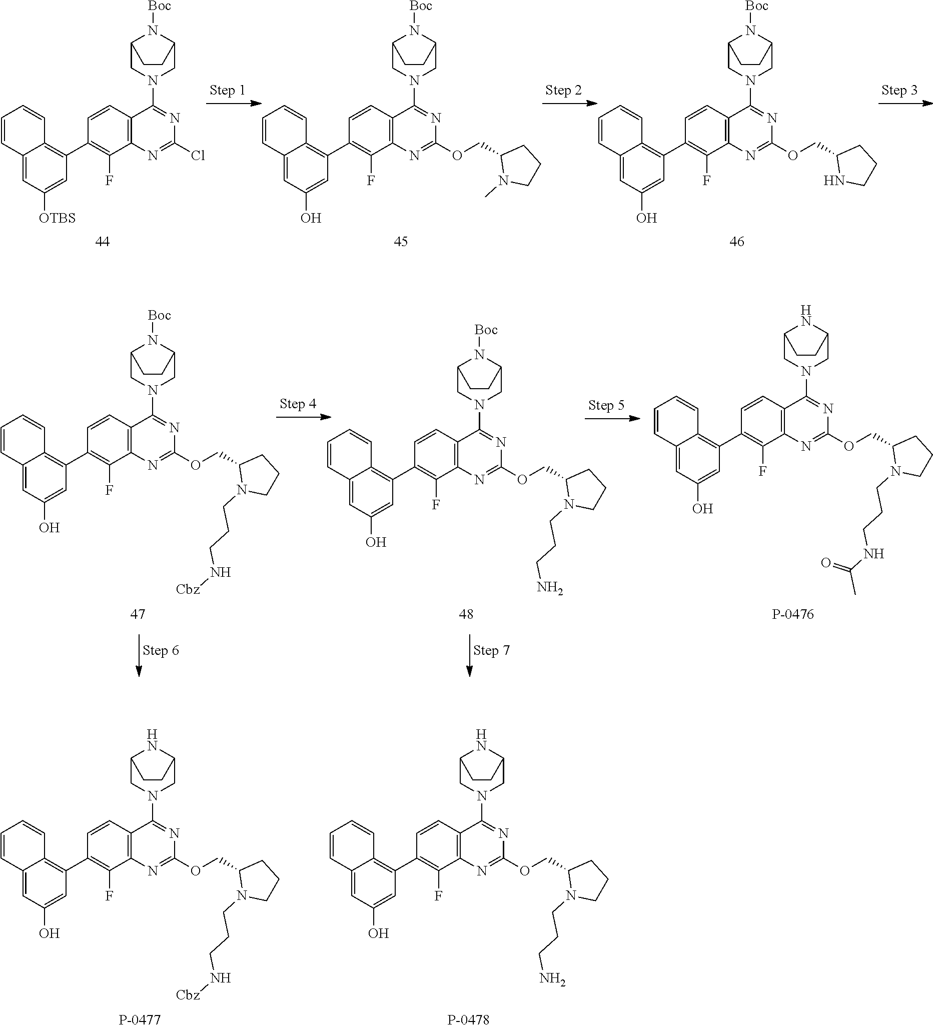

### Step 1: Tert-butyl 3-[2-[[(2S)-1-benzyloxycarbonylpyrrolidin-2-yl]methoxy]-8-fluoro-7-(3-hydroxy-1-naphthyl)quinazolin-4-yl]-3,8-diazabicyclo[3.2.1]octane-8-carboxylate (45)

In a 50 mL flask were added tert-butyl 3-[7-[3-[tert-butyl(dimethyl)silyl]oxy-1-naphthyl]-2-chloro-8-fluoro-quinazolin-4-yl]-3,8-diazabicyclo[3.2.1]octane-8-carboxylate (1, 1.0 g, 1.54 mmol), benzyl(2S)-2-(hydroxymethyl)pyrrolidine-1-carboxylate (1.81 g, 7.70 mmol), potassium carbonate (44, 28 g, 9.24 mmol), and RuPhos Pd G4 (393 mg, 0.462 mmol). DMF (6 mL) was added and the mixture was stirred at 95° C. for 1 hour. The reaction was directly purified by reverse phase chromatography (150 g C18 column, 0-100% MeCN in water with 0.1% formic acid). This purification provided tert-butyl 3-[2-[[(2S)-1-benzyloxycarbonylpyrrolidin-2-yl]methoxy]-8-fluoro-7-(3-hydroxy-1-naphthyl)quinazolin-4-yl]-3,8-diazabicyclo[3.2.1]octane-8-carboxylate (45, 590 mg). LCMS (M+H)+=734.4.

### Step 2: Tert-butyl 3-[8-fluoro-7-(3-hydroxy-1-naphthyl)-2-[[(2S)-pyrrolidin-2-yl]methoxy]quinazolin-4-yl]-3,8-diazabicyclo[3.2.1]octane-8-carboxylate (46)

Tert-butyl 3-[2-[[(2S)-1-benzyloxycarbonylpyrrolidin-2-yl]methoxy]-8-fluoro-7-(3-hydroxy-1-naphthyl)quinazolin-4-yl]-3,8-diazabicyclo[3.2.1]octane-8-carboxylate (45, 0.59 g, 0.804 mmol) was dissolved in 20 mL of ethyl acetate and palladium on carbon (10% Pd by weight, 100 mg) was added. The reaction mixture was subjected to hydrogen atmosphere (˜1 ATM, balloon) for 30 minutes at room temperature. The solution was filtered through celite and concentrated under reduced pressure to give tert-butyl 3-[8-fluoro-7-(3-hydroxy-1-naphthyl)-2-[[(2S)-pyrrolidin-2-yl]methoxy]quinazolin-4-yl]-3,8-diazabicyclo[3.2.1]octane-8-carboxylate (46, 482 mg). MS (ESI) [M+H⁺]⁺=600.3.

### Step 3 Tert-butyl 3-[2[[(2S)-1-[3-(benzyloxycarbonylamino)propyl]pyrrolidin-2-yl]methoxy]-8-fluoro-7-(3-hydroxy-1-naphthyl)quinazolin-4-yl]-3,8-diazabicyclo[3.2.1]octane-8-carboxylate (47)

Tert-butyl 3-[8-fluoro-7-(3-hydroxy-1-naphthyl)-2-[[(2S)-pyrrolidin-2-yl]methoxy]quinazolin-4-yl]-3,8-diazabicyclo[3.2.1]octane-8-carboxylate (46, 0.135 g, 0.225 mmol) was dissolved in dichloromethane (3 mL) and acetic acid (0.2 mL). Benzyl (3-oxopropyl)carbamate (56 mg, 0.27 mmol) and sodium triacetoxyborohydride (57 mg, 0.27 mmol) were added and the reaction was stirred at room temperature for 15 minutes. The reaction was concentrated and dissolved in 1 mL of DMF and 0.2 mL of water. This solution was directly purified by reverse phase chromatography (50 g C18 column, 0-80% MeCN in water with 0.1% formic acid) to provide tert-butyl 3-[2-[[(2S)-1-[3-(benzyloxycarbonylamino)propyl]pyrrolidin-2-yl]methoxy]-8-fluoro-7-(3-hydroxy-1-naphthyl)quinazolin-4-yl]-3,8-diazabicyclo[3.2.1]octane-8-carboxylate (47, 95 mg). MS (ESI) [M+H⁺]⁺=791.3.

### Step 4: Tert-butyl 3-[2-[[(2S)-1-(3-aminopropyl)pyrrolidin-2-yl]methoxy]-8-fluoro-7-(3-hydroxy-1-naphthyl)quinazolin-4-yl]-3,8-diazabicyclo[3.2.1]octane-8-carboxylate (48)

Tert-butyl 3-[2-[[(2S)-1-[3-(benzyloxycarbonylamino)propyl]pyrrolidin-2-yl]methoxy]-8-fluoro-7-(3-hydroxy-1-naphthyl)quinazolin-4-yl]-3,8-diazabicyclo[3.2.1]octane-8-carboxylate (47, 62 mg, 0.078 mmol) was dissolved in 10 mL of ethanol and palladium on carbon (10% Pd by weight, 50 mg) was added. The reaction mixture was subjected to hydrogen atmosphere (˜1 ATM, balloon) for 30 minutes at room temperature. The solution was filtered through celite and concentrated under reduced pressure to give tert-butyl 3-[2-[[(2S)-1-(3-aminopropyl)pyrrolidin-2-yl]methoxy]-8-fluoro-7-(3-hydroxy-1-naphthyl)quinazolin-4-yl]-3,8-diazabicyclo[3.2.1]octane-8-carboxylate (48, 51 mg). MS (ESI) [M+H⁺]⁺=657.3.

### Step 5: A-[3-[(2S)-2-[[4-(3,8-diazabicyclo[3.2.1]octan-3-yl)-8-fluoro-7-(3-hydroxy-1-naphthyl)quinazolin-2-yl]oxymethyl]pyrrolidin-1-yl]propyl]acetamide (P-0476)

Tert-butyl 3-[2-[[(2S)-1-(3-aminopropyl)pyrrolidin-2-yl]methoxy]-8-fluoro-7-(3-hydroxy-1-naphthyl)quinazolin-4-yl]-3,8-diazabicyclo[3.2.1]octane-8-carboxylate (48, 29 mg, 0.044 mmol) was dissolved in dichloromethane and triethylamine (35 mg, 0.44 mmol) and acetyl chloride (45 mg, 0.44 mmol) were added. The mixture was stirred at room temperature for 15 minutes. The reaction was concentrated and dissolved in 5 mL of TFA. This solution was left at room temperature for 4 days.

The reaction was directly purified by reverse phase chromatography (50 g C18 column, 0-50% MeCN in water with 0.1% formic acid). This purification provided N-[3-[(2S)-2-[[4-(3,8-diazabicyclo[3.2.1]octan-3-yl)-8-fluoro-7-(3-hydroxy-1-naphthyl)quinazolin-2-yl]oxymethyl]pyrrolidin-1-yl]propyl]acetamide (P-0476, 12 mg). MS (ESI) [M+H⁺]⁺=599.3.

### Step 6: Benzyl A-[3-[(2S)-2-[[4-(3,8-diazabicyclo[3.2.1]octan-3-yl)-8-fluoro-7-(3-hydroxy-1-naphthyl)quinazolin-2-yl]oxymethyl]pyrrolidin-1-yl]propyl]carbamate (P-0477)

Tert-butyl 3-[2-[[(2S)-1-[3-(benzyloxycarbonylamino)propyl]pyrrolidin-2-yl]methoxy]-8-fluoro-7-(3-hydroxy-1-naphthyl)quinazolin-4-yl]-3,8-diazabicyclo[3.2.1]octane-8-carboxylate (47, 33 mg, 0.042 mmol) was dissolved in 1 mL of trifluoroacetic acid and left to stand at room temperature for 15 minutes. The reaction was concentrated to give benzyl A-[3-[(2S)-2-[[4-(3,8-diazabicyclo[3.2.1]octan-3-yl)-8-fluoro-7-(3-hydroxy-1-naphthyl)quinazolin-2-yl]oxymethyl]pyrrolidin-1-yl]propyl]carbamate (P-0477, 29 mg). MS (ESI) [M+H⁺]⁺=691.3.

### Step 7: 4-[2-[[(2S)-1-(3-aminopropyl)pyrrolidin-2-yl]methoxy]-4-(3,8-diazabicyclo[3.2.1]octan-3-yl)-8-fluoro-quinazolin-7-yl]naphthalen-2-ol (P-0478)

Tert-butyl 3-[2-[[(2S)-1-(3-aminopropyl)pyrrolidin-2-yl]methoxy]-8-fluoro-7-(3-hydroxy-1-naphthyl)quinazolin-4-yl]-3,8-diazabicyclo[3.2.1]octane-8-carboxylate (48, 22 mg, 0.033 mmol) was dissolved in 1 mL of trifluoroacetic acid and left to stand at room temperature for 15 minutes. The reaction was concentrated to give 4-[2-[[(2S)-1-(3-aminopropyl)pyrrolidin-2-yl]methoxyl]-4-(3,8-diazabicyclo[3.2.1]octan-3-yl)-8-fluoro-quinazolin-7-yl]naphthalen-2-ol (P-0478, 19.5 mg). MS (ESI) [M+H⁺]⁺=557.3.

### Example 18

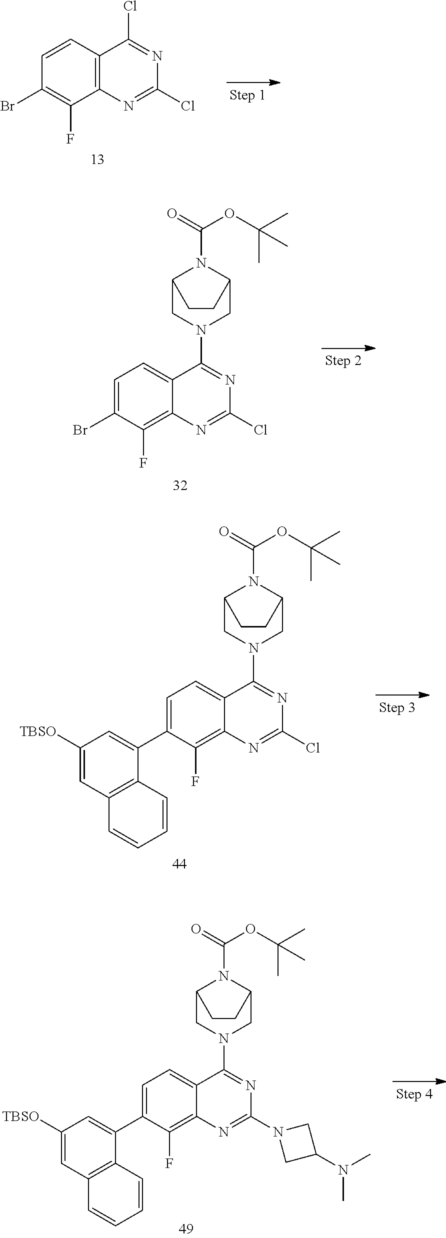

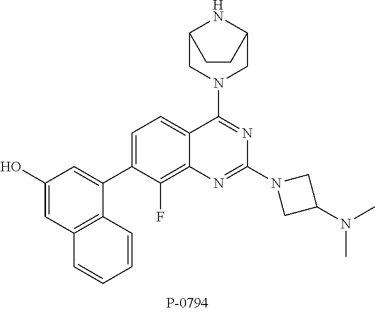 [SMILES: CN(C)C1CN(c2nc(N3CC4CCC(C3)N4)c3ccc(-c4cc(O)cc5ccccc45)c(F)c3n2)C1]

### Step 1: Tert-butyl (1R,5S)-3-(7-bromo-2-chloro-8-fluoroquinazolin-4-yl)-3,8-diazabicyclo[3.2.1]octane-8-carboxylate (32)

To a solution of 7-bromo-2,4-dichloro-8-fluoro-quinazoline (13, 300 mg, 1.01 mmol) in dioxane (3 mL), DIPEA (393 mg, 3.04 mmol) and tert-butyl 3,8-diazabicyclo[3.2.1]octane-8-carboxylate (237 mg, 1.16 mmol) were added. After stirring for 2 hours at room temperature, the reaction was quenched by the addition of H₂O, and the resulting was diluted with CH₂Cl₂. The layers were separated, and the aqueous layer was extracted with CH₂Cl₂. The combined organic layers were washed with brine, dried over anhydrous Na₂SO₄, and concentrated under reduced pressure. The residue was purified by prep-HPLC to afford the tert-butyl (1R,5S)-3-(7-bromo-2-chloro-8-fluoroquinazolin-4-yl)-3,8-diazabicyclo[3.2.1]octane-8-carboxylate (32, 450 mg). MS (ESI) [M+H]⁺=471.10.

### Step 2: Tert-butyl (1R,5S)-3-(7-(3-((tert-butyldimethylsilyl)oxy)naphthalen-1-yl)-2-chloro-8-fluoroquinazolin-4-yl)-3,8-diazabicyclo[3.2.1]octane-8-carboxylate (44)

To a solution of tert-butyl (1R,5S)-3-(7-bromo-2-chloro-8-fluoroquinazolin-4-yl)-3,8-diazabicyclo[3.2.1]octane-8-carboxylate (32, 3.3 g, 7 mmol) in dioxane/H₂O (4/1, 50 mL), tert-butyl-dimethyl-[[4-(4,4,5,5-tetramethyl-1,3,2-dioxaborolan-2-yl)-2-naphthyl]oxy]silane (3.2 g, 8.4 mmol), Na₂CO₃ (2.2 g, 21 mmol) and [1,1′-bis(diphenylphosphino)ferrocene]dichloropalladium(II) (511 mg, 0.7 mmol) were added at room temperature. After stirring for 3 hours at 90° C., the reaction mixture was concentrated and purified with normal phase chromatography to afford tert-butyl (1R,5S)-3-(7-(3-((tert-butyldimethylsilyl)oxy)naphthalen-1-yl)-2-chloro-8-fluoroquinazolin-4-yl)-3,8-diazabicyclo[3.2.1]octane-8-carboxylate (44, 2.5 g).

### Step 3: Tert-butyl (1R,5S)-3-(7-(3-((tert-butyldimethylsilyl)oxy)naphthalen-1-yl)-2-(3-(dimethylamino)azetidin-1-yl)-8-fluoroquinazolin-4-yl)-3,8-diazabicyclo[3.2.1]octane-8-carboxylate (49)

To a solution of tert-butyl (1R,5S)-3-(7-(3-((tert-butyldimethylsilyl)oxy)naphthalen-1-yl)-2-chloro-8-fluoroquinazolin-4-yl)-3,8-diazabicyclo[3.2.1]octane-8-carboxylate (44, 100 mg, 0.15 mmol) in MeCN (3 mL), N,N-dimethylazetidin-3-amine hydrochloride (400 mg, 0.3 mmol) and N-ethyl-N-isopropyl-propan-2-amine (100 mg, 0.77 mmol) were added. After stirring for 1 hour at 110° C. under microwave condition, the reaction mixture was concentrated and purified with reverse phase C18 column chromatography to afford tert-butyl (1R,5S)-3-(7-(3-((tert-butyldimethylsilyl)oxy)naphthalen-1-yl)-2-(3-(dimethylamino)azetidin-1-yl)-8-fluoroquinazolin-4-yl)-3,8-diazabicyclo[3.2.1]octane-8-carboxylate (49, 700 mg).

### Step 4: 4-(4-((1R,5S)-3,8-diazabicyclo[3.2.1]octan-3-yl)-2-(3-(dimethylamino)azetidin-1-yl)-8-fluoroquinazolin-7-yl)naphthalen-2-ol (P-0794)

To a solution of tert-butyl (1R,5S)-3-(7-(3-((tert-butyldimethylsilyl)oxy)naphthalen-1-yl)-2-(3-(dimethylamino)azetidin-1-yl)-8-fluoroquinazolin-4-yl)-3,8-diazabicyclo[3.2.1]octane-8-carboxylate (49, 20 mg, 0.03 mmol) in CH₂Cl₂ (10 mL), 4 N HCl in dioxane (1 mL) was added. After stirring for 2 hours at 75° C., the reaction mixture was concentrated under reduced pressure and washed with EtOAc to afford 4-(4-((1R,5S)-3,8-diazabicyclo[3.2.1]octan-3-yl)-2-(3-(dimethylamino)azetidin-1-yl)-8-fluoroquinazolin-7-yl)naphthalen-2-ol (P-0794, 22.6 mg). MS (ESI) [M+H]⁺=499.55.

### Example 19a

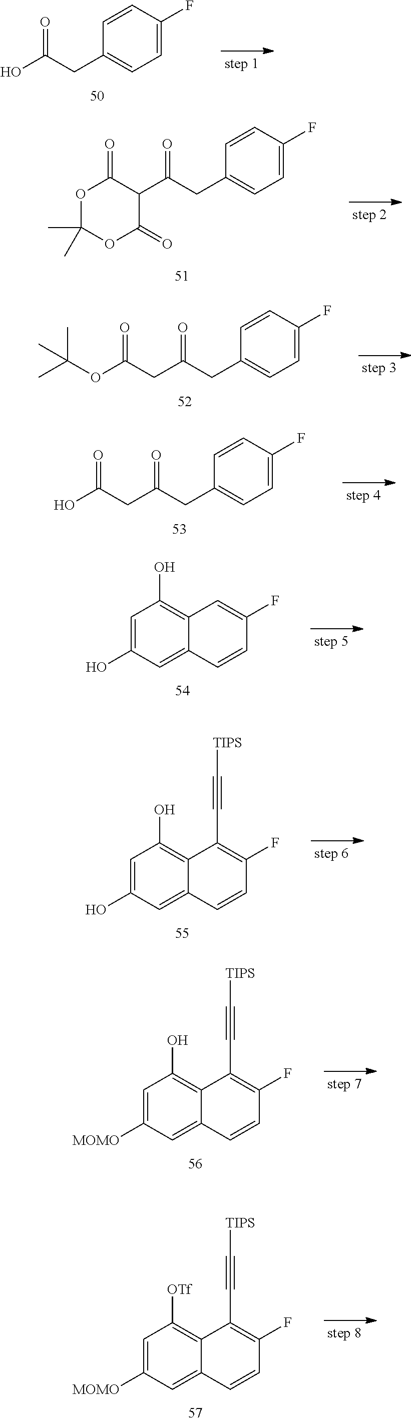

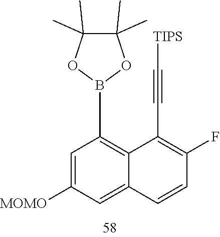

### Step 1: 5-(2-(4-Fluorophenyl)acetyl)-2,2-dimethyl-1,3-dioxane-4,6-dione (51)

To a solution of p-fluorophenylacetic acid (50, 50 g, 324 mmol), 2,2-dimethyl-1,3-dioxane-4,6-dione (51.4 g, 357 mmol) and DMAP (3.37 g, 27.6 mmol) in MeCN, DIPEA (90.1 g, 697 mmol) was slowly added below 45° C. under nitrogen atmosphere. After that, 2,2-dimethylpropanoyl chloride (43 g, 357 mmol) was added dropwise while still maintaining below 45° C. After stirring for 3 hours at 45° C., the reaction mixture was cooled to 0° C., and 1 M HCl was added. After additional stirring for 2 hours at 0° C., precipitate was formed. Filtration of the precipitate gave yellow solid, and the crude product was washed with the solution of MeCN/H₂O (1/4) to afford 5-[2-(4-fluorophenyl)acetyl]-2,2-dimethyl-1,3-dioxane-4,6-dione (51, 100 g). MS (ESI) [M+H]⁺=281.10.

### Step 2: Tert-butyl 4-(4-fluorophenyl)-3-oxobutanoate (52)

A mixture of 5-[2-(4-fluorophenyl)acetyl]-2,2-dimethyl-1,3-dioxane-4,6-dione (51, 96.3 g, 344 mmol) in t-BuOH (289 mL) was stirred for 1 hour at 90° C. After cooled to room temperature, the reaction mixture was concentrated under reduced pressure. The crude product was washed with petroleum ether to afford tert-butyl 4-(4-fluorophenyl)-3-oxobutanoate (52, 49.9 g). MS (ESI) [M+H]⁺=253.12.

### Step 3: 4-(4-Fluorophenyl)-3-oxobutanoic Acid (53)

To a solution of tert-butyl 4-(4-fluorophenyl)-3-oxobutanoate (52, 52.5 g, 208 mmol) in CH₂Cl₂ (105 mL), TFA (105 mL) was added dropwise at room temperature. After stirring for overnight at room temperature, the resulting mixture was concentrated under reduced pressure. The reaction was quenched by the addition of saturated aqueous NaHCO₃, and the resulting mixture was diluted with EtOAc. The layers were separated, and the aqueous layer was extracted with EtOAc. The combined organic layers were washed with brine, dried over anhydrous Na₂SO₄, and concentrated under reduced pressure to afford 4-(4-fluorophenyl)-3-oxobutanoic acid (53, 16.9 g). MS (ESI) [M+H]⁺=197.05.

### Step 4: 7-Fluoronaphthalene-1,3-diol (54)

A mixture of 4-(4-fluorophenyl)-3-oxobutanoic acid (53, 16.9 g, 86.2 mmol) in CF₃SO₃H (188 mL) was stirred overnight at room temperature. The reaction was quenched by the addition of saturated aqueous NaHCO₃, and the resulting mixture was diluted with EtOAc. The layers were separated, and the aqueous layer was extracted with EtOAc. The combined organic layers were washed with brine, dried over anhydrous Na₂SO₄, and concentrated under reduced pressure. The residue was purified by silica gel column chromatography to afford 7-fluoronaphthalene-1,3-diol (54, 11.3 g). MS (ESI) [M+H]⁺=179.00.

### Step 5: 7-Fluoro-8-((triisopropylsilyl)ethynyl)naphthalene-1,3-diol (55)

To a solution of 7-fluoronaphthalene-1,3-diol (54, 11.3 g, 63.4 mmol) and (2-bromoethynyl)triisopropylsilane (17.4 g, 66.6 mmol) in 1,4-dioxane (113 mL), dichloro(p-cymene) ruthenium(II) dimer (3.88 g, 6.34 mmol) and potassium acetate (12.5 g, 127 mmol) were added at room temperature. After stirring for 2 hours at 110° C. under nitrogen atmosphere, the resulting mixture was filtered, and the filter cake was washed with EtOAc. The filtrate was concentrated under reduced pressure. The residue was purified by silica gel column chromatography to afford 7-fluoro-8-[2-(triisopropylsilyl)ethynyl]naphthalene-1,3-diol (55, 18.3 g). MS (ESI) [M+H]⁺=395.20.

### Step 6: 7-Fluoro-3-(methoxymethoxy)-8-((triisopropylsilyl)ethynyl)naphthalen-1-ol (56)

To a solution of 7-fluoro-8-[2-(triisopropylsilyl)ethynyl]naphthalene-1,3-diol (55, 18.3 g, 51 mmol) in CH₂Cl₂ (190 mL), chloromethyl methyl ether (6.16 g, 76.6 mmol) and DIPEA (19.8 g, 153 mmol) were added dropwise at 0° C. After stirring for 3 hours at room temperature, the reaction was quenched by the addition of NH₃.H₂O, and the resulting mixture was diluted with CH₂Cl₂. The layers were separated, and the aqueous layer was extracted with CH₂Cl₂. The combined organic layers were washed with brine, dried over anhydrous Na₂SO₄, and concentrated under reduced pressure. The residue was purified by silica gel column chromatography to afford 7-fluoro-3-(methoxymethoxy)-8-((triisopropylsilyl)ethynyl)naphthalen-1-ol (56, 13.1 g). MS (ESI) [M+H]⁺=403.20.

### Step 7: 7-Fluoro-3-(methoxymethoxy)-8-((triisopropylsilyl)ethynyl)naphthalen-1-yl trifluoromethanesulfonate (57)

To a stirred solution of 7-fluoro-3-(methoxymethoxy)-8-[2-(triisopropylsilyl) ethynyl]naphthalen-1-ol (56, 13.1 g, 32.5 mmol) in CH₂Cl₂ (130 mL), DIPEA (12.6 g, 97.6 mmol) and Tf₂O (13.8 g, 48.8 mmol) were added dropwise at −40° C. under nitrogen atmosphere. After stirring for 1 hour at −40° C. under nitrogen atmosphere, the reaction was quenched by the addition of H₂O at 0° C. The resulting mixture was extracted with CH₂Cl₂ and the combined organic layers were washed with brine, dried over anhydrous Na₂SO₄. After filtration, the filtrate was concentrated under reduced pressure. The residue was purified by silica gel column chromatography to afford 7-fluoro-3-(methoxymethoxy)-8-((triisopropylsilyl)ethynyl)naphthalen-1-yl trifluoromethanesulfonate (57, 9.9 g). MS (ESI) [M+H]⁺=535.20.

### Step 8: ((2-Fluoro-6-(methoxymethoxy)-8-(4,4,5,5-tetramethyl-1,3,2-dioxaborolan-2-yl)naphthalen-1-yl)ethynyl)triisopropylsilane (58)

To a solution of 7-fluoro-3-(methoxymethoxy)-8-((triisopropylsilyl)ethynyl)naphthalen-1-yl trifluoromethanesulfonate (57, 5 g, 9.35 mmol) and bis(pinacolato)diboron (7.12 g, 28.1 mmol) in toluene (50 mL), [1,1′-bis(diphenylphosphino)ferrocene]dichloropalladium(II), complex with dichloromethane (760 mg, 0.94 mmol) and potassium acetate (2.75 g, 28.1 mmol) were added at room temperature under nitrogen atmosphere. After stirring for 4 hours at 130° C. under nitrogen atmosphere, the resulting mixture was filtered, and the filter cake was washed with EtOAc. The filtrate was concentrated under reduced pressure, and the residue was purified by silica gel column chromatography to afford ((2-fluoro-6-(methoxymethoxy)-8-(4,4,5,5-tetramethyl-1,3,2-dioxaborolan-2-yl)naphthalen-1-yl)ethynyl)triisopropylsilane (58, 2.41 g). MS (ESI) [M+H]⁺=513.40.

### Example 19b

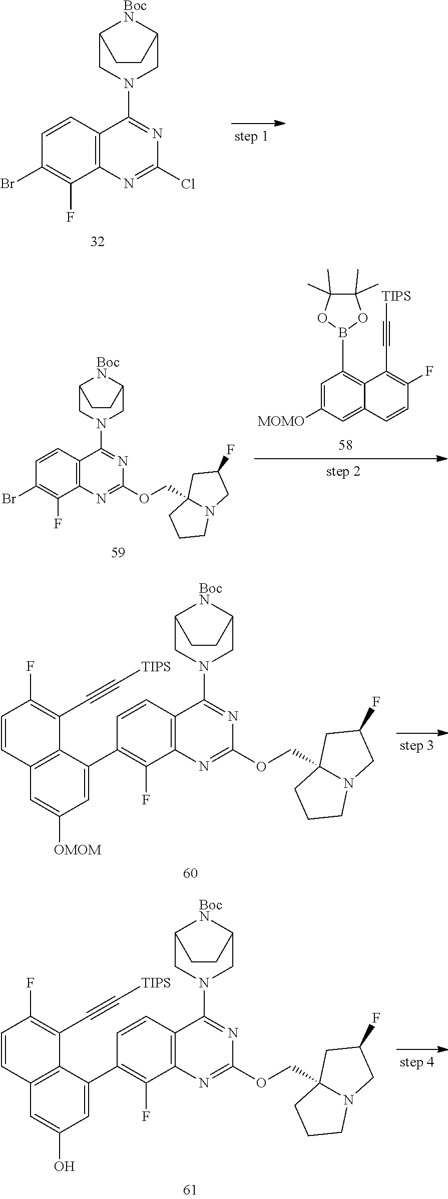

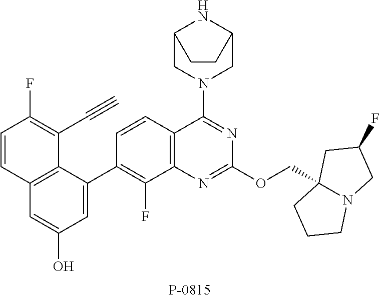 [SMILES: C#Cc1c(F)ccc2cc(O)cc(-c3ccc4c(N5CC6CCC(C5)N6)nc(OC[C@@]56CCCN5C[C@H](F)C6)nc4c3F)c12]

### Step 1: Tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (59)

To a solution of tert-butyl 3-(7-bromo-2-chloro-8-fluoro-quinazolin-4-yl)-3,8-diazabicyclo[3.2.1]octane-8-carboxylate (32, 2 g, 4.24 mmol) and [(2R,8S)-2-fluoro-1,2,3,5,6,7-hexahydropyrrolizin-8-yl]methanol (1.35 g, 8.48 mmol) in DMSO (20 mL), KF (500 mg, 8.61 mmol) was added. After stirring for overnight at 90° C. under nitrogen, and run the additional stirring for 6 hours at 120° C. The reaction was filtered, concentrated, and purified with reverse phase C18 column chromatography to afford tert-butyl (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (59, 1.2 g).

### Step 2: Tert-butyl (1R,5S)-3-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60)

To a solution of (1R,5S)-3-(7-bromo-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (59, 200 mg, 0.33 mmol) and {2-[2-fluoro-6-(methoxymethoxy)-8-(4,4,5,5-tetramethyl-1,3,2-dioxaborolan-2-yl)naphthalen-1-yl]ethynyl}triisopropylsilane (58, 207 mg, 0.4 mmol) in dioxane/H₂O (5/1, 6 mL), CataCXium A Pd G₂ (23 mg, 0.03 mmol), CataCXium (12 mg, 0.03 mmol) and Cs₂CO₃ (219 mg, 0.67 mmol) were added at room temperature under nitrogen atmosphere. After stirring for overnight at 90° C. under nitrogen atmosphere, the reaction mixture was cooled to room temperature, and concentrated under reduced pressure. The residue was diluted with EtOAc, and washed with H₂O. The organic layer was concentrated under reduced pressure, and the residue was purified by prep-TLC to afford tert-butyl (1R,5S)-3-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60, 210 mg). MS (ESI) [M+H]⁺=900.46.

### Step 3: 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-((triisopropylsilyl)ethynyl)naphthalen-2-ol (61)

4 M HCl in dioxane (10 mL) was added to tert-butyl (1R,5S)-3-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60, 200 mg, 0.22 mmol) at room temperature. After stirring for 2 hours at room temperature, the reaction mixture was concentrated under reduced pressure to afford 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-((triisopropylsilyl)ethynyl)naphthalen-2-ol (61, 150 mg). MS (ESI) [M+H]⁺=738.39.

### Step 4: 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol (P-0815)

To a solution of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-6-fluoro-5-((triisopropylsilyl)ethynyl)naphthalen-2-ol (61, 20 mg, 0.0270 mmol) in DMF (0.5 mL), CsF (41 mg, 0.27 mmol) was added at room temperature. After stirring for overnight at room temperature, the reaction mixture was filtered and washed with DMF. The filtrate was concentrated under reduced pressure. The crude product was purified by prep-HPLC to afford 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol (P-0815, 4.1 mg). MS (ESI) [M+H]⁺=600.30.

### Example 20

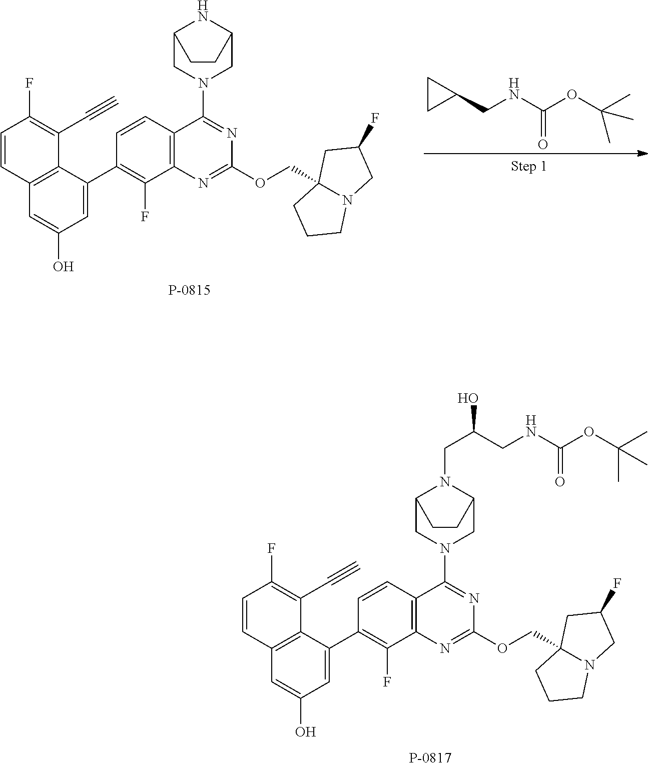

### Step 1: Tert-butyl ((S)-3-((1R,5S)-3-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-hydroxypropyl)carbamate (P-0817)

To a solution of 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)quinazolin-7-yl)-5-ethynyl-6-fluoronaphthalen-2-ol (P-0815, 20 mg, 0.03 mmol) and tert-butyl (S)-(oxiran-2-ylmethyl)carbamate (6 mg, 0.03 mmol) in 1-BuOH (0.4 mL), DIPEA (9 mg, 0.06 mmol) was added at room temperature. After stirring for 3 hours at 80° C., the reaction was cooled to room temperature and concentrated under reduced pressure. The residue was purified by Prep-HPLC with the following conditions (Column: XBridge Prep C18 OBD Column, 30×50 mm, 5 μm, 13 nm; Mobile Phase A: water (10 mmol/L, NH₄HCO₃), Mobile Phase B: MeCN; Flow rate: 60 mL/min; Gradient: 5% B to 5% B in 2 min, 5% B to 18% B in 2.5 min, 18% B to 50% B in 9.5 min, 50% B; wave Length: 220 nm; RT1 (min): 9.4) to afford tert-butyl ((S)-3-((1R,5S)-3-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octan-8-yl)-2-hydroxypropyl)carbamate (P-0817, 6.4 mg). MS (ESI) [M+H]⁺=773.35.

### Example 21a

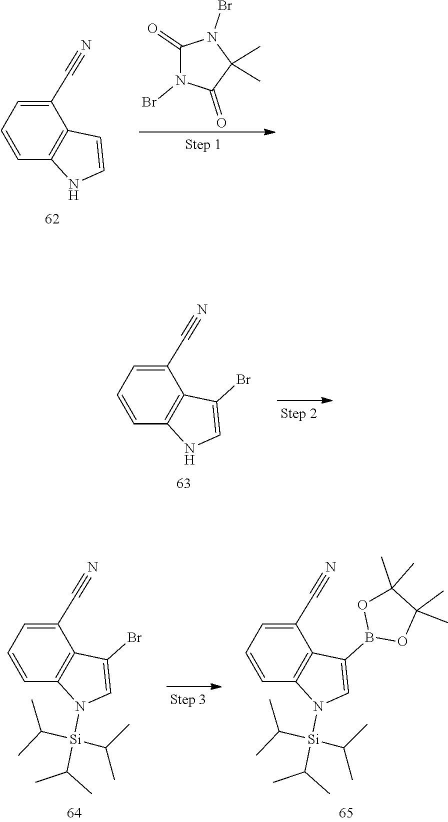 [SMILES: N#Cc1cccc2[nH]ccc12 ; CC1(C)C(=O)N(Br)C(=O)N1Br ; N#Cc1cccc2[nH]cc(Br)c12 ; CC(C)[Si](C(C)C)(C(C)C)n1cc(Br)c2c(C#N)cccc21 ; CC(C)[Si](C(C)C)(C(C)C)n1cc(B2OC(C)(C)C(C)(C)O2)c2c(C#N)cccc21]

### Step 1:3-Bromo-1H-indole-4-carbonitrile (63)

To a solution of 1H-indole-4-carbonitrile (62, 960 mg, 6.4 mmol) in 15 mL dioxane, 1,3-dibromo-5,5-dimethyl-imidazolidine-2,4-dione (1.00 g, 3.5 mmol) in 5 mL dioxane was added dropwise at 0° C. After stirring for 10 min at room temperature, the reaction was quenched by the addition of saturated aqueous NaHCO₃, and the resulting mixture was diluted with EtOAc. The layers were separated, and the aqueous layer was extracted with EtOAc. The combined organic layers were washed with brine, dried over anhydrous MgSO₄, and concentrated under reduced pressure. The residue was isolated via 40 g silica column, eluting with EtOAc/hexanes (product eluted ˜30% EtOAc). The fractions identified by TLC (20% EtOAc/hexane; bromo product Rf˜0.2) were pooled and concentrated to afford the 3-bromo-1H-indole-4-carbonitrile (63, 1.35 g). MS (ESI) [M+H]⁺=222.9.

### Step 2: 3-Bromo-1-(triisopropylsilyl)-1H-indole-4-carbonitrile (64)

To a solution of 3-bromo-1H-indole-4-carbonitrile (63, 1.35 g, 6.13 mmol) in 20 mL THF, NaH (60% in mineral oil, 316 mg, 7.97 mmol) was added portion-wise as a solid in 0 to 5° C. After stirring for over 10 min at 0 to 5° C., triisopropylsilyl chloride (1.43 g, 7.36 mmol) in 5 mL THF was added dropwise. After stirring for 10 min at 0 to 5° C., the reaction was quenched by the addition of saturated aqueous NH₄Cl/H₂O (1/1), and the resulting was diluted with EtOAc. The layers were separated, and the aqueous layer was extracted with EtOAc. The combined organic layers were washed with brine, dried over anhydrous MgSO₄, and concentrated under reduced pressure. The residue was isolated via 40 g silica, eluting with EtOAc/hexane (product elutes ˜15% EtOAc). Fractions identified as product by TLC (20% EtOAc/hexane; product Rf˜0.7) to afford 3-bromo-1-(triisopropylsilyl)-1H-indole-4-carbonitrile (64, 2.52 g). MS (ESI) [M+H]⁺=378.95.

### Step 3: 3-(4,4,5,5-tetramethyl-1,3,2-dioxaborolan-2-yl)-1-(triisopropylsilyl)-1H-indole-4-carbonitrile (65)

To a cooled (−78° C.) solution of 3-bromo-1-(triisopropylsilyl)-1H-indole-4-carbonitrile (64, 1.26 g, 3.34 mmol) in 50 mL THF was added dropwise M-BuLi (2.5 M in hexanes, 2.2 mL, 5.5 mmol). After stirring for 1 hour at −78° C., the reaction mixture was treated with an Pinacolborane (1.28 g, 10.1 mmol) in 5 mL THF. After stirring for 30 min, the reaction was quenched by addition of 5 mL of MeOH, and the resulting mixture was diluted with EtOAc. The combined organic layers were washed with saturated aqueous NH₄Cl, and the resulting was diluted with EtOAc. The layers were separated, and the aqueous layer was extracted with EtOAc. The combined organic layers were washed with brine, dried over anhydrous MgSO₄, and concentrated under reduced pressure. The residue was isolated via 40 g silica, eluting with EtOAc/hexane (product eluted ˜10% EtOAc/hexane). The fractions identified by TLC (20% EtOAc/hexane; product Rf˜0.5) were pooled and concentrated to afford 3-(4,4,5,5-tetramethyl-1,3,2-dioxaborolan-2-yl)-1-(triisopropylsilyl)-1H-indole-4-carbonitrile (65, 0.68 g). MS (ESI) [M+H⁺]⁺=425.15.

### Example 21b

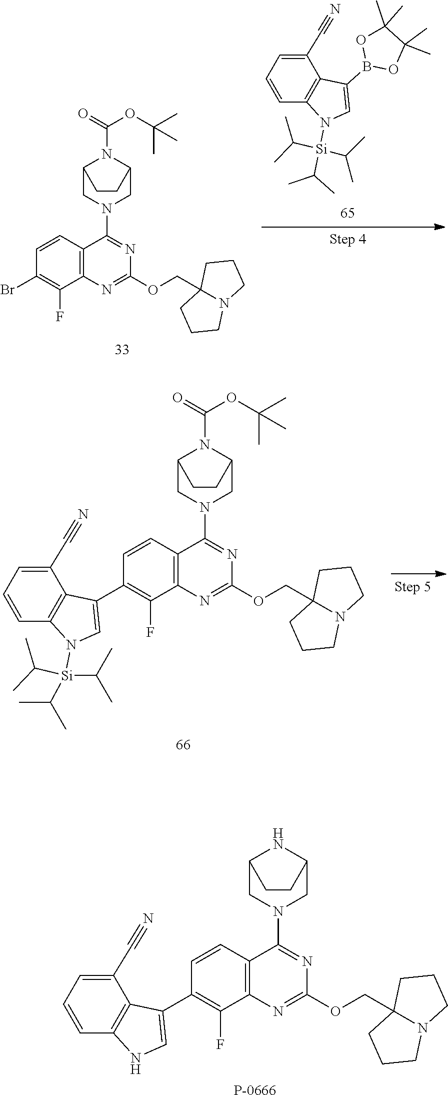

### Step 4: Tert-butyl (1R,5S)-3-(7-(4-cyano-1-(triisopropylsilyl)-1H-indol-3-yl)-8-fluoro-2-((tetrahydro-1H-pyrrolizin-7a(5H)-yl)methoxy)quinazolin-4-yl)-3,8-diazabicyclo[3.2.1]octane-8-carboxylate (65)

Tert-butyl (1R,5S)-3-(7-bromo-8-fluoro-2-((tetrahydro-1H-pyrrolizin-7a(5H)-yl)methoxy)quinazolin-4-yl)-3,8-diazabicyclo[3.2.1]octane-8-carboxylate (33, 80 mg, 0.13 mmol), 3-(4,4,5,5-tetramethyl-1,3,2-dioxaborolan-2-yl)-1-(triisopropylsilyl)-1H-indole-4-carbonitrile (65, 91 mg, 0.20 mmol), Na₂CO₃ (52 mg, 0.49 mmol) and Pd(PPh₃)₄ (26 mg, 0.02 mmol) were dissolved in 1.6 mL dioxane and 0.4 mL water (2 mL of 20% H₂O/dioxane). After stirring for 1 hour at 80° C., the reaction mixture was cooled to room temperature, filtered to remove solids, and concentrated to residue. The residue was dissolved in 2 mL DMSO and the Boc/des-Tips product isolated via 13 g flash C18, eluting with MeCN/H₂O (0.1% formic acid). The fractions containing Boc/des-Tips product were pooled frozen, and lyophilized to afford tert-butyl (1R,5S)-3-(7-(4-cyano-1-(triisopropylsilyl)-1H-indol-3-yl)-8-fluoro-2-((tetrahydro-1H-pyrrolizin-7a(5H)-yl)methoxy)quinazolin-4-yl)-3,8-diazabicyclo[3.2.1]octane-8-carboxylate (66, 57 mg).

### Step 5: 3-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)-1H-indole-4-carbonitrile (P-0666)

To a tert-butyl (1R,5S)-3-(7-(4-cyano-1-(triisopropylsilyl)-1H-indol-3-yl)-8-fluoro-2-((tetrahydro-1H-pyrrolizin-7a(5H)-yl)methoxy)quinazolin-4-yl)-3,8-diazabicyclo[3.2.1]octane-8-carboxylate (66, 57 mg, 0.07 mmol), 50% TFA in CH₂Cl₂ (2 mL) was added. After stirring for 30 min at room temperature, the reaction mixture was concentrated to residue, dissolved in 4 mL H₂O, and product isolated via prep-LC, eluting with MeCN/H₂O (0.1% TFA). Fractions containing product were pooled, frozen, and lyophilized to afford 3-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)-1H-indole-4-carbonitrile (P-0666, 43 mg). MS (ESI) [M+H]⁺=538.60.

### Example 22

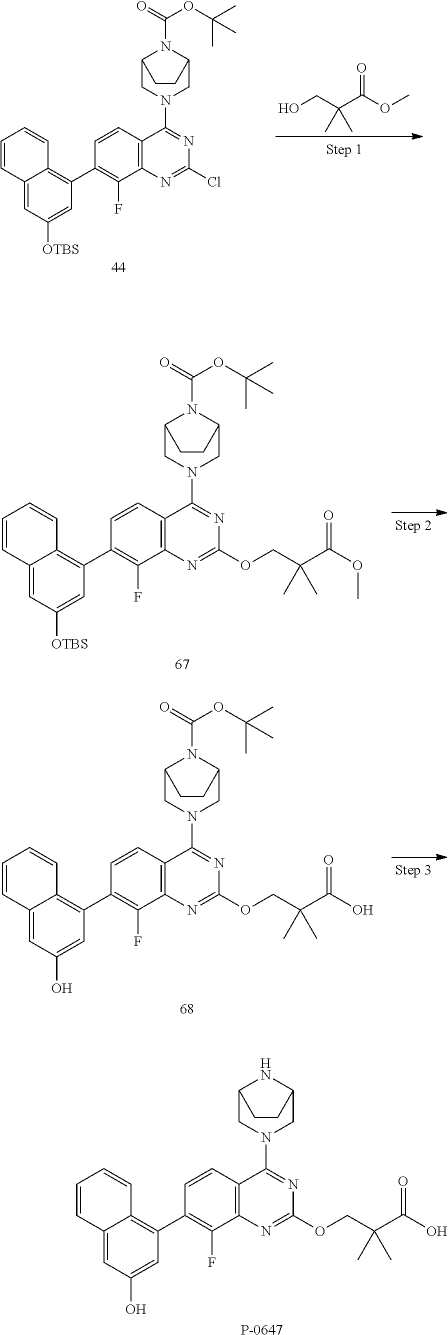

### Step 1: Tert-butyl (1R,5S)-3-(7-(3-((tert-butyldimethylsilyl)oxy)naphthalen-1-yl)-8-fluoro-2-(3-methoxy-2,2-dimethyl-3-oxopropoxy)quinazolin-4-yl)-3,8-diazabicyclo[3.2.1]octane-8-carboxylate (2)

The mixture of tert-butyl (1R,5S)-3-(7-(3-((tert-butyldimethylsilyl)oxy)naphthalen-1-yl)-2-chloro-8-fluoroquinazolin-4-yl)-3,8-diazabicyclo[3.2.1]octane-8-carboxylate (44, 90 mg, 0.13 mmol), methyl 3-hydroxy-2,2-dimethylpropanoate (36.6 mg, 0.27 mmol), anhydrous Cs₂CO₃ (90.4 mg, 0.27 mmol) and PdRuPhos G4 catalyst (11.9 mg, 0.01 mmol) were purged with N₂ gas and then added 2 mL dioxane. After stirring for 1 hour at 90° C. 1 mL water and 1 mL MeOH were added to the reaction mixture. After additional stirring for 30 min at room temperature, removed solvent under reduced pressure and the residue was purified by RP-HPLC to afford tert-butyl (1R,5S)-3-(7-(3-((tert-butyldimethylsilyl)oxy)naphthalen-1-yl)-8-fluoro-2-(3-methoxy-2,2-dimethyl-3-oxopropoxy)quinazolin-4-yl)-3,8-diazabicyclo[3.2.1]octane-8-carboxylate (67, 58 mg).

### Step 2: 3-((4-((1R,5S)-8-(tert-butoxycarbonyl)-3,8-diazabicyclo[3.2.1]octan-3-yl)-8-fluoro-7-(3-hydroxynaphthalen-1-yl)quinazolin-2-yl)oxy)-2,2-dimethylpropanoic acid (68)

To a solution of afford tert-butyl (1R,5S)-3-(7-(3-((tert-butyldimethylsilyl)oxy)naphthalen-1-yl)-8-fluoro-2-(3-methoxy-2,2-dimethyl-3-oxopropoxy)quinazolin-4-yl)-3,8-diazabicyclo[3.2.1]octane-8-carboxylate (67, 58 mg, 0.07 mmol) in 2 mL MeOH/THF (1/1), 1 N LiOH in H₂O (1 mL) were added. After stirring for 4 hours at room temperature, the reaction mixture through a short silica pad with 30% MeOH in CH₂Cl₂. Concentrated the filtrate under reduced pressure and purified the residue by RP-HPLC to afford 3-((4-((1R,5S)-3,8-diazabicyclo[3.2.1]octan-3-yl)-8-fluoro-7-(3-hydroxynaphthalen-1-yl)quinazolin-2-yl)oxy)-2,2-dimethylpropanoic acid (68, 30 mg).

### Step 3: 3-((4-((1R,5S)-3,8-diazabicyclo[3.2.1]octan-3-yl)-8-fluoro-7-(3-hydroxynaphthalen-1-yl)quinazolin-2-yl)oxy)-2,2-dimethylpropanoic acid (P-0647)

To a solution of 3-((4-((1R,5S)-3,8-diazabicyclo[3.2.1]octan-3-yl)-8-fluoro-7-(3-hydroxynaphthalen-1-yl)quinazolin-2-yl)oxy)-2,2-dimethylpropanoic acid (68, 30 mg, 0.04 mmol) in CH₂Cl₂ (1 mL), 0.2 mL TFA was added. After stirring for 1 hour at room temperature, the reaction was concentrated under reduced pressure. The residue was purified by RP-HPLC to afford 3-((4-((1R,5S)-3,8-diazabicyclo[3.2.1]octan-3-yl)-8-fluoro-7-(3-hydroxynaphthalen-1-yl)quinazolin-2-yl)oxy)-2,2-dimethylpropanoic acid (P-0647, 19 mg). MS (ESI) [M+H]⁺=517.60.

### Example 23

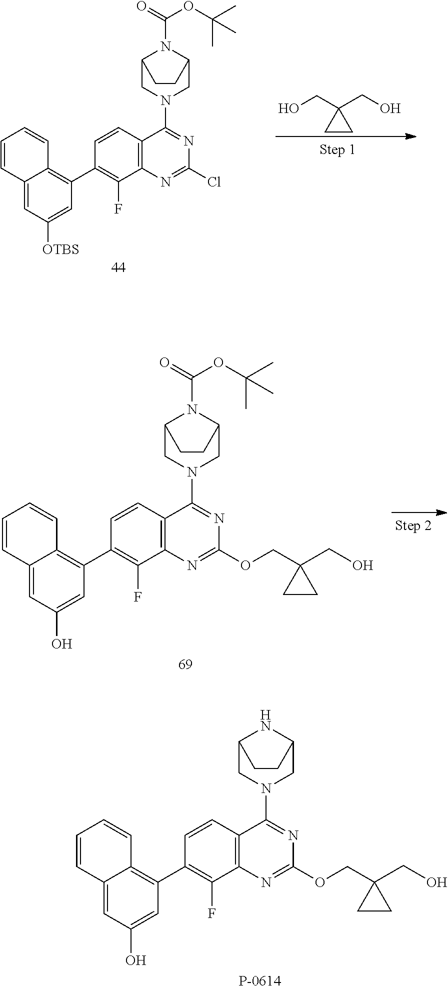

### Step 1: Tert-butyl (1R,5S)-3-(8-fluoro-2-((1-(hydroxymethyl)cyclopropyl)methoxy)-7-(3-hydroxynaphthalen-1-yl)quinazolin-4-yl)-3,8-diazabicyclo[3.2.1]octane-8-carboxylate (69)

A mixture of tert-butyl (1R,5S)-3-(7-(3-((tert-butyldimethylsilyl)oxy)naphthalen-1-yl)-2-chloro-8-fluoroquinazolin-4-yl)-3,8-diazabicyclo[3.2.1]octane-8-carboxylate (44, 90 mg, 0.13 mmol), cyclopropane-1,1-diyldimethanol (28.3 mg, 0.27 mmol), anhydrous Cs₂CO₃ (90.4 mg, 0.27 mmol) and PdRuPhos G4 catalyst (11.9 mg, 0.01 mmol) was purged with N₂ gas and then added 2 mL dioxane. After stirring for 1 hour at 90° C. 1 mL water and 1 mL MeOH were added to the reaction mixture. After additional stirring for 30 min at room temperature, the reaction was concentrated under reduced pressure. The residue was purified by RP-HPLC to afford tert-butyl (1R,5S)-3-(8-fluoro-2-((1-(hydroxymethyl)cyclopropyl)methoxy)-7-(3-hydroxynaphthalen-1-yl)quinazolin-4-yl)-3,8-diazabicyclo[3.2.1]octane-8-carboxylate (69, 52 mg).

### Step 2: 4-(4-((1R,5S)-3,8-diazabicyclo[3.2.1]octan-3-yl)-8-fluoro-2-((l (hydroxymethyl)cyclopropyl)methoxy)quinazolin-7-yl)naphthalen-2-ol (3)

To a solution of tert-butyl (1R,5S)-3-(8-fluoro-2-((1-(hydroxymethyl)cyclopropyl)methoxy)-7-(3-hydroxynaphthalen-1-yl)quinazolin-4-yl)-3,8-diazabicyclo[3.2.1]octane-8-carboxylate (69, 52 mg, 0.08 mmol) in CH₂Cl₂ (1 mL), 0.2 mL TFA was added. After stirring for 1 hour at room temperature, the reaction was concentrated under reduced pressure. The residue was purified by RP-HPLC to afford 4-(4-((1R,5S)-3,8-diazabicyclo[3.2.1]octan-3-yl)-8-fluoro-2-((l (hydroxymethyl)cyclopropyl)methoxy)quinazolin-7-yl)naphthalen-2-ol (P-0614, 44 mg). MS (ESI) [M+H]⁺=501.60.

### Example 24

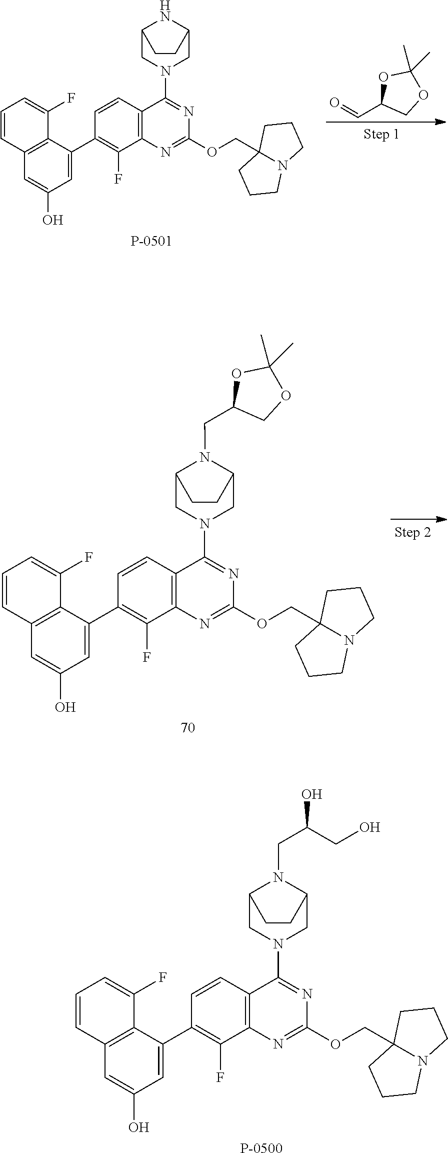

### Step 1: 4-(4-((1R,5S)-8-(((R)-2,2-dimethyl-1,3-dioxolan-4-yl)methyl)-3,8-diazabicyclo[3.2.1]octan-3-yl)-8-fluoro-2-((tetrahydro-1H-pyrrolizin-7a(5H)-yl)methoxy)quinazolin-7-yl)-5-fluoronaphthalen-2-ol (2)

To a mixture of 4-(4-((1R,5S)-3,8-diazabicyclo[3.2.1]octan-3-yl)-8-fluoro-2-((tetrahydro-1H-pyrrolizin-7a(5H)-yl)methoxy)quinazolin-7-yl)-5-fluoronaphthalen-2-ol (P-0501, 60 mg, 0.10 mmol), (S)-2,2-dimethyl-1,3-dioxolane-4-carbaldehyde (140 mg, 1.08 mmol), formic acid (0.5 mg, 0.01 mmol) in THF (2 mL) was added at room temperature. After stirring for 1 hour at 40° C., NaBH₄ (12 mg, 0.32 mmol) was added in portions at room temperature. After stirring for overnight at room temperature, the reaction was quenched by addition of saturated aqueous NH₄Cl (5 mL) at room temperature, and the resulting mixture was diluted with EtOAc. The aqueous layer was extracted with EtOAc, and the combined organic layer was concentrated under reduced pressure. The residue was purified by Prep-HPLC with the following conditions (Column: XBridge Shield RP18 OBD Column, 19×150 mm, 5 μm; Mobile Phase A: water (10 mmol/L, NH₄HCO₃), Mobile Phase B: MeCN; Flow rate: 25 mL/min; Gradient: 34% B to 61% B in 7 min, 61% B; wave Length: 220 nm; RT1 (min): 6.45) to afford 4-(4-((1R,5S)-8-(((R)-2,2-dimethyl-1,3-dioxolan-4-yl)methyl)-3,8-diazabicyclo[3.2.1]octan-3-yl)-8-fluoro-2-((tetrahydro-1H-pyrrolizin-7a(5H)-yl)methoxy)quinazolin-7-yl)-5-fluoronaphthalen-2-ol (70, 12.1 mg). MS (ESI) [M+H]⁺=672.

### Step 2: (R)-3-((1R,5S)-3-(8-fluoro-7-(8-fluoro-3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)propane-1,2-diol

To a solution of 4-(4-((1R,5S)-8-(((R)-2,2-dimethyl-1,3-dioxolan-4-yl)methyl)-3,8-diazabicyclo[3.2.1]octan-3-yl)-8-fluoro-2-((tetrahydro-1H-pyrrolizin-7a(5H)-yl)methoxy)quinazolin-7-yl)-5-fluoronaphthalen-2-ol (70, 12 mg, 0.01 mmol) in 0.3 mL dioxane, 4 M HCl in dioxane (0.3 mL) was added at room temperature. After stirring for 1 hour at room temperature, the resulting mixture was concentrated under reduced pressure. The residue was purified by trituration with hexane (0.5 mL) to afford (R)-3-((1R,5S)-3-(8-fluoro-7-(8-fluoro-3-hydroxynaphthalen-1-yl)-2-((tetrahydro-1H-pyrrolizin-7a(5H)-yl)methoxy)quinazolin-4-yl)-3,8-diazabicyclo[3.2.1]octan-8-yl)propane-1,2-diol (P-0501, 10.1 mg). MS (ESI) [M+H]⁺=632.15.

All compounds in Tables IA and IB listed below can be made according to the synthetic examples described in this disclosure, and by making any necessary substitutions of starting materials that the skilled artisan would be able to obtain either commercially or otherwise.

TABLE IA

| P# | Structure | Name | (MH)+ |
|---|---|---|---|
| P-0001 | 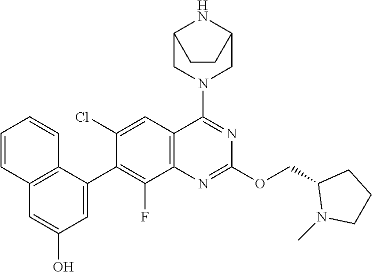 [SMILES: CN1CCC[C@H]1COc1nc(N2CC3CCC(C2)N3)c2cc(Cl)c(-c3cc(O)cc4ccccc34)c(F)c2n1] | 4-(4-((1R,5S)-3,8- diazabicyclo[3.2.1]octan-3-yl)-6- chloro-8-fluoro-2-(((S)-1- methylpyrrolidin-2- yl)methoxy)quinazolin-7- yl)naphthalen-2-ol | 548.3 |
| P-0002 | 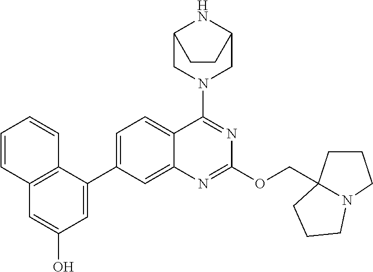 [SMILES: Oc1cc(-c2ccc3c(N4CC5CCC(C4)N5)nc(OCC45CCCN4CCC5)nc3c2)c2ccccc2c1] | 4-(4-((1R,5S)-3,8- diazabicyclo[3.2.1]octan-3-yl)-2- ((tetrahydro-1H-pyrrolizin-7a(5H)- yl)methoxy)quinazolin-7- yl)naphthalen-2-ol | 522.1 |
| P-0003 | 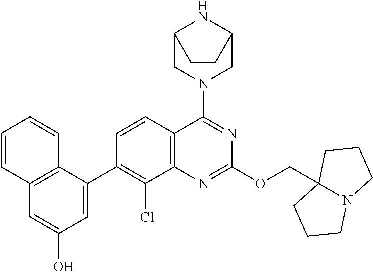 [SMILES: Oc1cc(-c2ccc3c(N4CC5CCC(C4)N5)nc(OCC45CCCN4CCC5)nc3c2Cl)c2ccccc2c1] | 4-(4-((1R,5S)-3,8- diazabicyclo[3.2.1]octan-3-yl)-8- chloro-2-((tetrahydro-1H- pyrrolizin-7a(5H)- yl)methoxy)quinazolin-7- yl)naphthalen-2-ol | 556.3 |
| P-0004 | 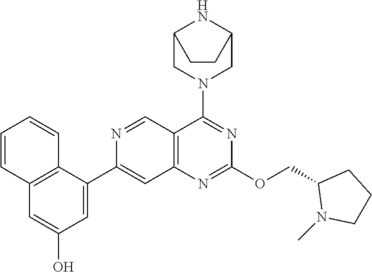 [SMILES: CN1CCC[C@H]1COc1nc(N2CC3CCC(C2)N3)c2cnc(-c3cc(O)cc4ccccc34)cc2n1] | 4-(4-((1R,5S)-3,8- diazabicyclo[3.2.1]octan-3-yl)-2- (((S)-1-methylpyrrolidin-2- yl)methoxy)pyrido[4,3-d]pyrimidin- 7-yl)naphthalen-2-ol | 497.2 |
| P-0005 | 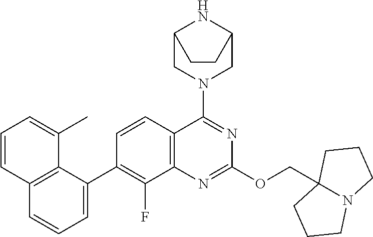 [SMILES: Cc1cccc2cccc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(8-methylnaphthalen-1-yl)- 2-((tetrahydro-1H-pyrrolizin- 7a(5H)-yl)methoxy)quinazoline | 538.2 |
| P-0006 | 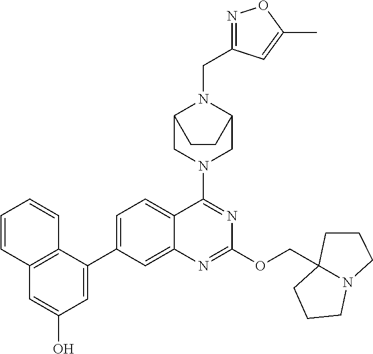 [SMILES: Cc1cc(CN2C3CCC2CN(c2nc(OCC45CCCN4CCC5)nc4cc(-c5cc(O)cc6ccccc56)ccc24)C3)no1] | 4-(4-((1R,5S)-8-((5-methylisoxazol- 3-yl)methyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 617.10 |
| P-0007 | 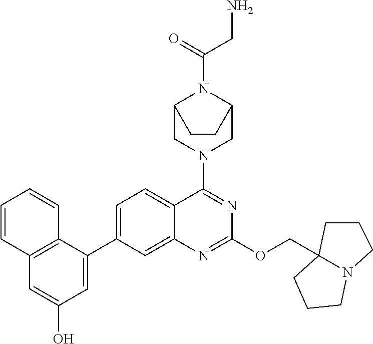 [SMILES: NCC(=O)N1C2CCC1CN(c1nc(OCC34CCCN3CCC4)nc3cc(-c4cc(O)cc5ccccc45)ccc13)C2] | 2-amino-1-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethan-1-one | 577.3 |
| P-0008 | 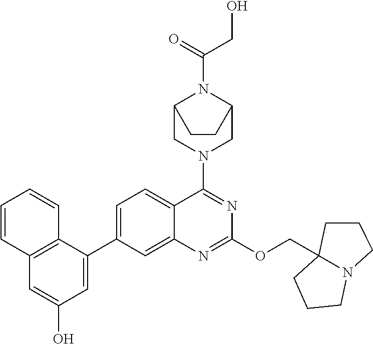 [SMILES: O=C(CO)N1C2CCC1CN(c1nc(OCC34CCCN3CCC4)nc3cc(-c4cc(O)cc5ccccc45)ccc13)C2] | 2-hydroxy-1-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethan-1-one | 580.7 |
| P-0009 | 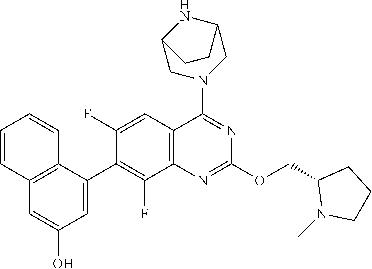 [SMILES: CN1CCC[C@H]1COc1nc(N2CC3CCC(C2)N3)c2cc(F)c(-c3cc(O)cc4ccccc34)c(F)c2n1] | 4-(4-((1R,5S)-3,8- diazabicyclo[3.2.1]octan-3-yl)-6,8- difluoro-2-(((S)-1-methylpyrrolidin- 2-yl)methoxy)quinazolin-7- yl)naphthalen-2-ol | 532.3 |
| P-0010 | 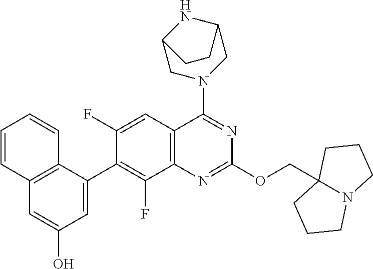 [SMILES: Oc1cc(-c2c(F)cc3c(N4CC5CCC(C4)N5)nc(OCC45CCCN4CCC5)nc3c2F)c2ccccc2c1] | 4-(4-((1R,5S)-3,8- diazabicyclo[3.2.1]octan-3-yl)-6,8- difluoro-2-((tetrahydro-1H- pyrrolizin-7a(5H)- yl)methoxy)quinazolin-7- yl)naphthalen-2-ol | 558.3 |
| P-0011 | 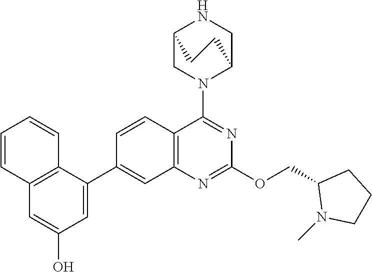 [SMILES: CN1CCC[C@H]1COc1nc(N2C[C@@H]3CC[C@H]2CN3)c2ccc(-c3cc(O)cc4ccccc34)cc2n1] | 4-(4-((1S,4S)-2,5- diazabicyclo[2.2.2]octan-2-yl)-2- (((S)-1-methylpyrrolidin-2- yl)methoxy)quinazolin-7- yl)naphthalen-2-ol | 496.20 |
| P-0012 | 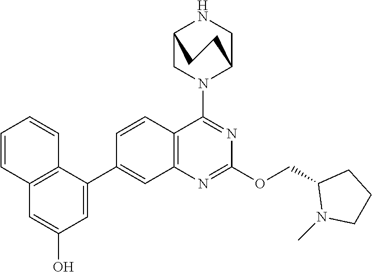 [SMILES: CN1CCC[C@H]1COc1nc(N2C[C@H]3CC[C@@H]2CN3)c2ccc(-c3cc(O)cc4ccccc34)cc2n1] | 4-(4-((1R,4R)-2,5- diazabicyclo[2.2.2]octan-2-yl)-2- (((S)-1-methylpyrrolidin-2- yl)methoxy)quinazolin-7- yl)naphthalen-2-ol | 496.2 |
| P-0013 | 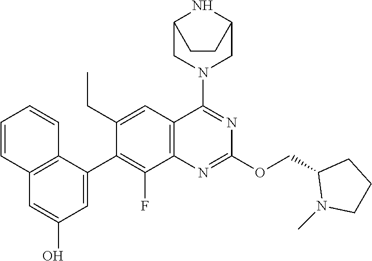 [SMILES: CCc1cc2c(N3CC4CCC(C3)N4)nc(OC[C@@H]3CCCN3C)nc2c(F)c1-c1cc(O)cc2ccccc12] | 4-(4-((1R,5S)-3,8- diazabicyclo[3.2.1]octan-3-yl)-6- ethyl-8-fluoro-2-(((S)-1- methylpyrrolidin-2- yl)methoxy)quinazolin-7- yl)naphthalen-2-ol | 542.3 |
| P-0014 | 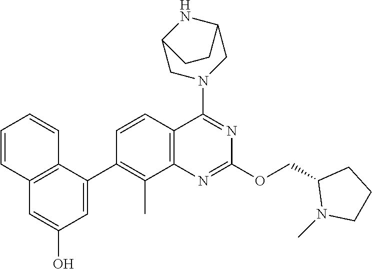 [SMILES: Cc1c(-c2cc(O)cc3ccccc23)ccc2c(N3CC4CCC(C3)N4)nc(OC[C@@H]3CCCN3C)nc12] | 4-(4-((1R,5S)-3,8- diazabicyclo[3.2.1]octan-3-yl)-8- methyl-2-(((S)-1-methylpyrrolidin- 2-yl)methoxy)quinazolin-7- yl)naphthalen-2-ol | 510.3 |
| P-0015 | 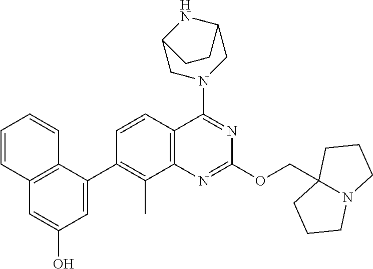 [SMILES: Cc1c(-c2cc(O)cc3ccccc23)ccc2c(N3CC4CCC(C3)N4)nc(OCC34CCCN3CCC4)nc12] | 4-(4-((1R,5S)-3,8- diazabicyclo[3.2.1]octan-3-yl)-8- methyl-2-((tetrahydro-1H- pyrrolizin-7a(5H)- yl)methoxy)quinazolin-7- yl)naphthalen-2-ol | 536.4 |
| P-0016 | 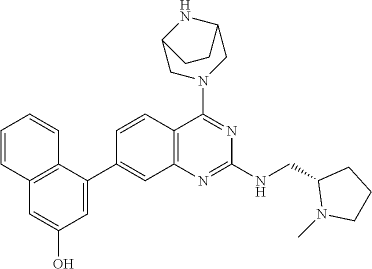 [SMILES: CN1CCC[C@H]1CNc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- ((((S)-1-methylpyrrolidin-2- yl)methyl)amino)quinazolin-7- yl)naphthalen-2-ol | 495.4 |
| P-0017 | 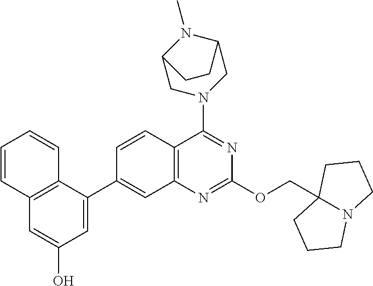 [SMILES: CN1C2CCC1CN(c1nc(OCC34CCCN3CCC4)nc3cc(-c4cc(O)cc5ccccc45)ccc13)C2] | 4-(4-((1R,5S)-8-methyl-3,8- diazabicyclo[3.2.1]octan-3-yl)-2- ((tetrahydro-1H-pyrrolizin-7a(5H)- yl)methoxy)quinazolin-7- yl)naphthalen-2-ol | 534.4 |
| P-0018 | 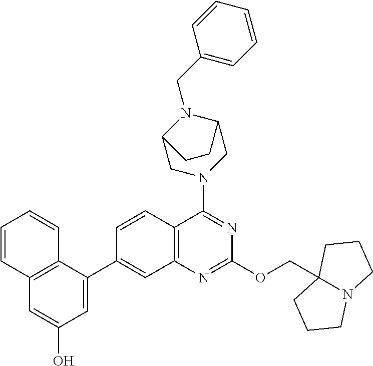 [SMILES: Oc1cc(-c2ccc3c(N4CC5CCC(C4)N5Cc4ccccc4)nc(OCC45CCCN4CCC5)nc3c2)c2ccccc2c1] | 4-(4-((1R,5S)-8-benzyl-3,8- diazabicyclo[3.2.1]octan-3-yl)-2- ((tetrahydro-1H-pyrrolizin-7a(5H)- yl)methoxy)quinazolin-7- yl)naphthalen-2-ol | 612.4 |
| P-019 | 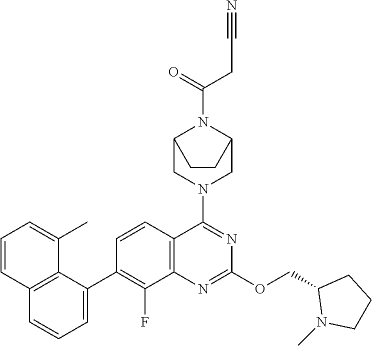 [SMILES: Cc1cccc2cccc(-c3ccc4c(N5CC6CCC(C5)N6C(=O)CC#N)nc(OC[C@@H]5CCCN5C)nc4c3F)c12] | 3-((1R,5S)-3-(8-fluoro-7-(8- methylnaphthalen-1-yl)-2-(((RS)-1- methylpyrrolidin-2- yl)methoxy)quinazolin-4-yl)-3,8- diazabicyclo[3.2.1]octan-8-yl)-3- oxopropanenitrile | 579.4 |
| P-0020 | 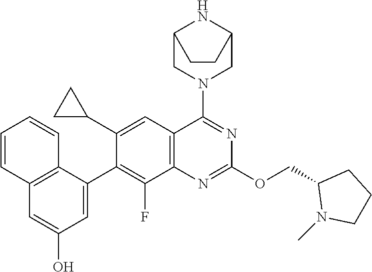 [SMILES: CN1CCC[C@H]1COc1nc(N2CC3CCC(C2)N3)c2cc(C3CC3)c(-c3cc(O)cc4ccccc34)c(F)c2n1] | 4-(4-((1R,5S)-3,8- diazabicyclo[3.2.1]octan-3-yl)-6- cyclopropyl-8-fluoro-2-(((S)-1- methylpyrrolidin-2- yl)methoxy)quinazolin-7- yl)naphthalen-2-ol | 554.3 |
| P-0021 | 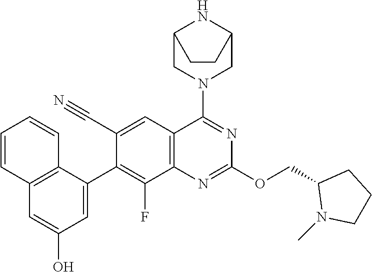 [SMILES: CN1CCC[C@H]1COc1nc(N2CC3CCC(C2)N3)c2cc(C#N)c(-c3cc(O)cc4ccccc34)c(F)c2n1] | 4-((1R,5S)-3,8- diazabicyclo[3.2.1]octan-3-yl)-8- fluoro-7-(3-hydroxynaphthalen-1- yl)-2-(((S)-1-methylpyrrolidin-2- yl)methoxy)quinazoline-6- carbonitrile | 539.3 |
| P-0022 | 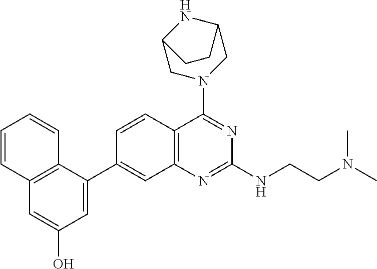 [SMILES: CN(C)CCNc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- ((2- (dimethylamino)ethyl)amino)quinaz- olin-7-yl)naphthalen-2-ol | 469.3 |
| P-0023 | 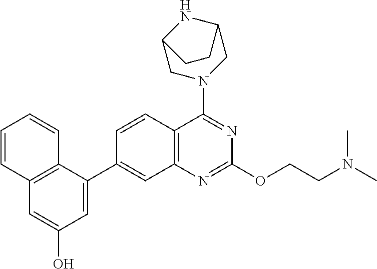 [SMILES: CN(C)CCOc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2-(2- (dimethylamino)ethoxy)quinazolin- 7-yl)naphthalen-2-ol | 470.3 |
| P-0024 | 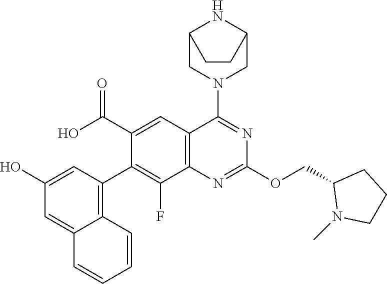 [SMILES: CN1CCC[C@H]1COc1nc(N2CC3CCC(C2)N3)c2cc(C(=O)O)c(-c3cc(O)cc4ccccc34)c(F)c2n1] | 4-((1R,5S)-3,8- diazabicyclo[3.2.1]octan-3-yl)-8- fluoro-7-(3-hydroxynaphthalen-1- yl)-2-(((S)-1-methylpyrrolidin-2- yl)methoxy)quinazoline-6- carboxylic acid | 558.3 |
| P-0025 | 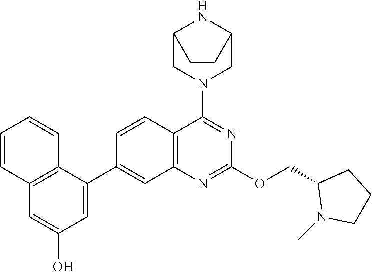 [SMILES: CN1CCC[C@H]1COc1nc(N2CC3CCC(C2)N3)c2ccc(-c3cc(O)cc4ccccc34)cc2n1] | 4-(4-((1R,5S)-8- azabicyclo[3.2.1]octan-3-yl)-2- (((S)-1-methylpyrrolidin-2- yl)methoxy)quinazolin-7- yl)naphthalen-2-ol | 495.2 |
| P-0026 | 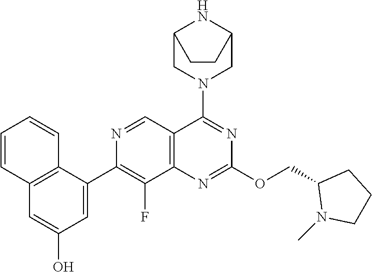 [SMILES: CN1CCC[C@H]1COc1nc(N2CC3CCC(C2)N3)c2cnc(-c3cc(O)cc4ccccc34)c(F)c2n1] | 4-(4-((1R,5S)-3,8- diazabicyclo[3.2.1]octan-3-yl)-8- fluoro-2-(((S)-1-methylpyrrolidin- 2-yl)methoxy)pyrido[4,3- d]pyrimidin-7-yl)naphthalen-2-ol | 515.3 |
| P-0027 | 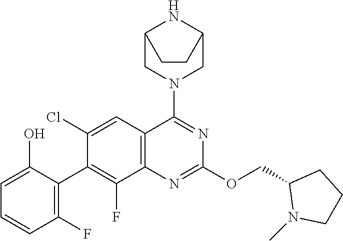 [SMILES: CN1CCC[C@H]1COc1nc(N2CC3CCC(C2)N3)c2cc(Cl)c(-c3c(O)cccc3F)c(F)c2n1] | 2-(4-((1R,5S)-3,8- diazabicyclo[3.2.1]octan-3-yl)-6- chloro-8-fluoro-2-(((S)-1- methylpyrrolidin-2- yl)methoxy)quinazolin-7-yl)-3- fluorophenol | 516.2 |
| P-0028 | 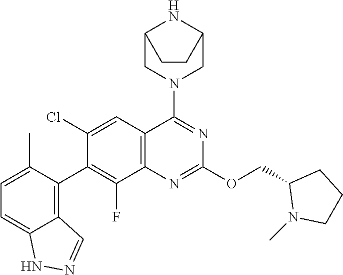 [SMILES: Cc1ccc2[nH]ncc2c1-c1c(Cl)cc2c(N3CC4CCC(C3)N4)nc(OC[C@@H]3CCCN3C)nc2c1F] | 4-((1R,5S)-3,8- diazabicyclo[3.2.1]octan-3-yl)-6- chloro-8-fluoro-7-(5-methyl-1H- indazol-4-yl)-2-(((S)-1- methylpyrrolidin-2- yl)methoxy)quinazoline | 536.2 |
| P-0029 | 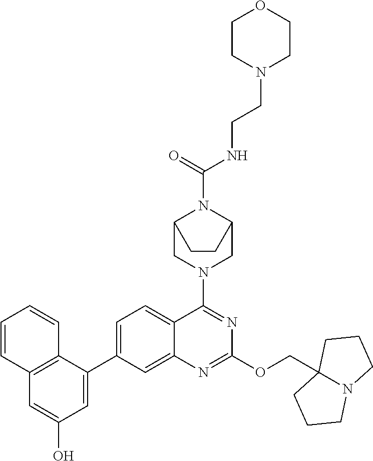 [SMILES: O=C(NCCN1CCOCC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N-(2- morpholinoethyl)-3,8- diazabicyclo[3.2.1]octane-8- carboxamide | 678.30 |
| P-0030 | 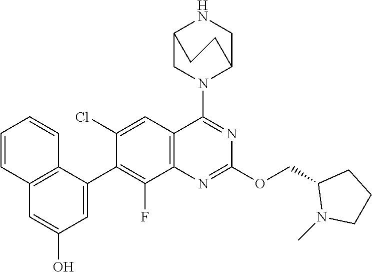 [SMILES: CN1CCC[C@H]1COc1nc(N2CC3CCC2CN3)c2cc(Cl)c(-c3cc(O)cc4ccccc34)c(F)c2n1] | 4-(4-(2,5-diazabicyclo[2.2.2]octan- 2-yl)-6-chloro-8-fluoro-2-(((S)-1- methylpyrrolidin-2- yl)methoxy)quinazolin-7- yl)naphthalen-2-ol | 548.3 |
| P-0031 | 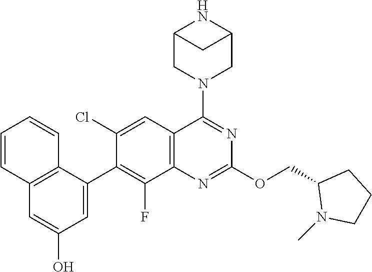 [SMILES: CN1CCC[C@H]1COc1nc(N2CC3CC(C2)N3)c2cc(Cl)c(-c3cc(O)cc4ccccc34)c(F)c2n1] | 4-(4-(3,6- diazabicyclo[3.1.1]heptan-3-yl)-6- chloro-8-fluoro-2-(((S)-1- methylpyrrolidin-2- yl)methoxy)quinazolin-7- yl)naphthalen-2-ol | 534.3 |
| P-0032 | 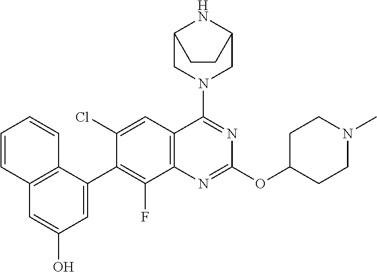 [SMILES: CN1CCC(Oc2nc(N3CC4CCC(C3)N4)c3cc(Cl)c(-c4cc(O)cc5ccccc45)c(F)c3n2)CC1] | 4-(4-((1R,5S)-3,8- diazabicyclo[3.2.1]octan-3-yl)-6- chloro-8-fluoro-2-((1- methylpiperidin-4- yl)oxy)quinazolin-7-yl)naphthalen- 2-ol | 548.3 |
| P-0033 | 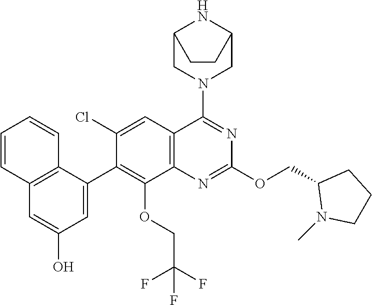 [SMILES: CN1CCC[C@H]1COc1nc(N2CC3CCC(C2)N3)c2cc(Cl)c(-c3cc(O)cc4ccccc34)c(OCC(F)(F)F)c2n1] | 4-(4-((1R,5S)-3,8- diazabicyclo[3.2.1]octan-3-yl)-6- chloro-2-(((S)-1-methylpyrrolidin- 2-yl)methoxy)-8-(2,2,2- trifluoroethoxy)quinazolin-7- yl)naphthalen-2-ol | 628.3 |
| P-0034 | 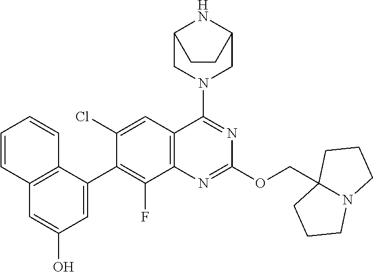 [SMILES: Oc1cc(-c2c(Cl)cc3c(N4CC5CCC(C4)N5)nc(OCC45CCCN4CCC5)nc3c2F)c2ccccc2c1] | 4-(4-((1R,5S)-3,8- diazabicyclo[3.2.1]octan-3-yl)-6- chloro-8-fluoro-2-((tetrahydro-1H- pyrrolizin-7a(5H)- yl)methoxy)quinazolin-7- yl)naphthalen-2-ol | 574.3 |
| P-0035 | 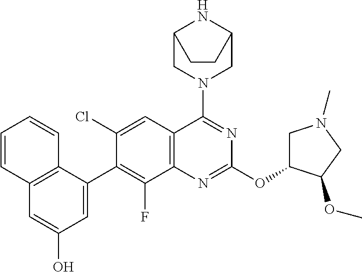 [SMILES: CO[C@@H]1CN(C)C[C@H]1Oc1nc(N2CC3CCC(C2)N3)c2cc(Cl)c(-c3cc(O)cc4ccccc34)c(F)c2n1] | 4-(4-((1R,5S)-3,8- diazabicyclo[3.2.1]octan-3-yl)-6- chloro-8-fluoro-2-(((3R,4R)-4- methoxy-1-methylpyrrolidin-3- yl)oxy)quinazolin-7-yl)naphthalen- 2-ol | 564.3 |
| P-0036 | 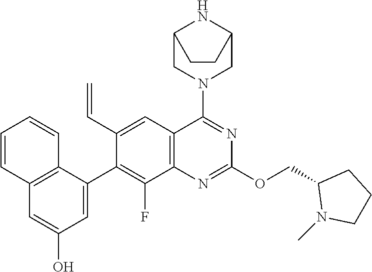 [SMILES: C=Cc1cc2c(N3CC4CCC(C3)N4)nc(OC[C@@H]3CCCN3C)nc2c(F)c1-c1cc(O)cc2ccccc12] | 4-(4-((1R,5S)-3,8- diazabicyclo[3.2.1]octan-3-yl)-8- fluoro-2-(((S)-1-methylpyrrolidin- 2-yl)methoxy)-6-vinylquinazolin-7- yl)naphthalen-2-ol | 540.3 |
| P-0037 | 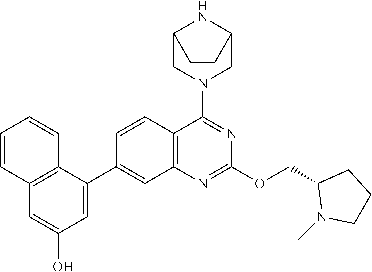 [SMILES: CN1CCC[C@H]1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- (((S)-1-methylpyrrolidin-2- yl)methoxy)quinazolin-7- yl)naphthalen-2-ol | 496.3 |
| P-0038 | 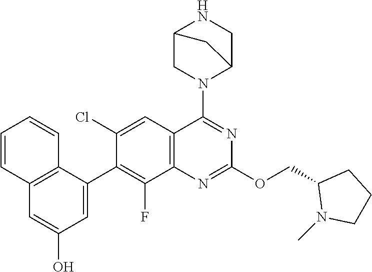 [SMILES: CN1CCC[C@H]1COc1nc(N2CC3CC2CN3)c2cc(Cl)c(-c3cc(O)cc4ccccc34)c(F)c2n1] | 4-(4-(2,5- diazabicyclo[2.2.1]heptan-2-yl)-6- chloro-8-fluoro-2-(((S)-1- methylpyrrolidin-2- yl)methoxy)quinazolin-7- yl)naphthalen-2-ol | 534.1 |
| P-0039 | 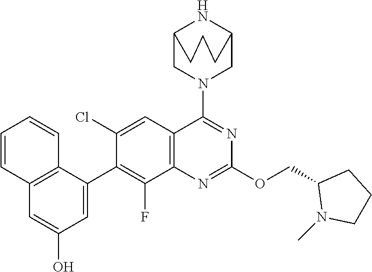 [SMILES: CN1CCC[C@H]1COc1nc(N2CC3CCCC(C2)N3)c2cc(Cl)c(-c3cc(O)cc4ccccc34)c(F)c2n1] | 4-(4-(3,9-diazabicyclo[3.3.1]nonan- 3-yl)-6-chloro-8-fluoro-2-(((S)-1- methylpyrrolidin-2- yl)methoxy)quinazolin-7- yl)naphthalen-2-ol | 562.1 |
| P-0040 | 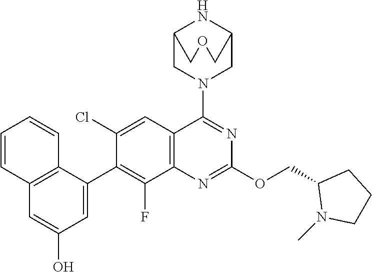 [SMILES: CN1CCC[C@H]1COc1nc(N2CC3COCC(C2)N3)c2cc(Cl)c(-c3cc(O)cc4ccccc34)c(F)c2n1] | 4-(4-(3-oxa-7,9- diazabicyclo[3.3.1]nonan-7-yl)-6- chloro-8-fluoro-2-(((S)-1- methylpyrrolidin-2- yl)methoxy)quinazolin-7- yl)naphthalen-2-ol | 564.0 |
| P-0041 | 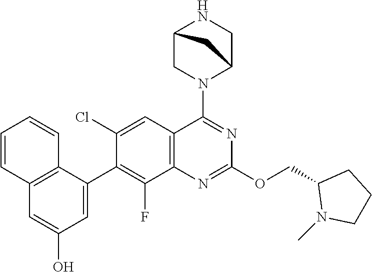 [SMILES: CN1CCC[C@H]1COc1nc(N2C[C@H]3C[C@@H]2CN3)c2cc(Cl)c(-c3cc(O)cc4ccccc34)c(F)c2n1] | 4-(4-((1R,4R)-2,5- diazabicyclo[2.2.1]heptan-2-yl)-6- chloro-8-fluoro-2-(((S)-1- methylpyrrolidin-2- yl)methoxy)quinazolin-7- yl)naphthalen-2-ol | 534.0 |
| P-0042 | 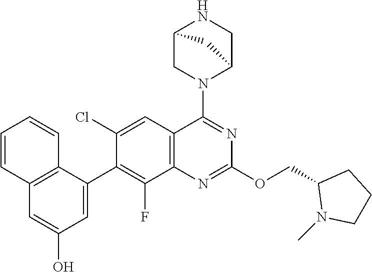 [SMILES: CN1CCC[C@H]1COc1nc(N2C[C@@H]3C[C@H]2CN3)c2cc(Cl)c(-c3cc(O)cc4ccccc34)c(F)c2n1] | 4-(4-((1S,4S)-2,5- diazabicyclo[2.2.1]heptan-2-yl)-6- chloro-8-fluoro-2-(((S)-1- methylpyrrolidin-2- yl)methoxy)quinazolin-7- yl)naphthalen-2-ol | 534.00 |
| P-0043 | 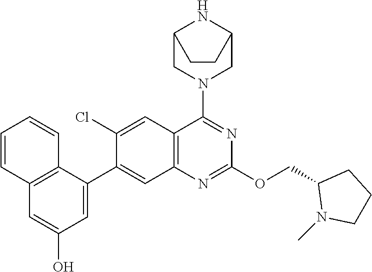 [SMILES: CN1CCC[C@H]1COc1nc(N2CC3CCC(C2)N3)c2cc(Cl)c(-c3cc(O)cc4ccccc34)cc2n1] | 4-(4-((1R,5S)-3,8- diazabicyclo[3.2.1]octan-3-yl)-6- chloro-2-(((S)-1-methylpyrrolidin- 2-yl)methoxy)quinazolin-7- yl)naphthalen-2-ol | 530.2 |
| P-0044 | 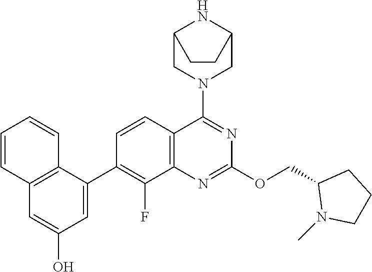 [SMILES: CN1CCC[C@H]1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(((S)-1-methylpyrrolidin- 2-yl)methoxy)quinazolin-7- yl)naphthalen-2-ol | 514.2 |
| P-0045 | 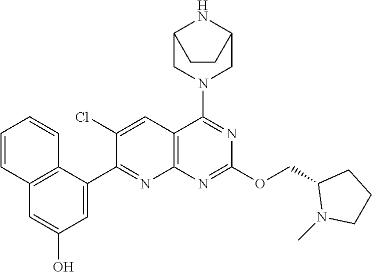 [SMILES: CN1CCC[C@H]1COc1nc(N2CC3CCC(C2)N3)c2cc(Cl)c(-c3cc(O)cc4ccccc34)nc2n1] | 4-(4-((1R,5S)-3,8- diazabicyclo[3.2.1]octan-3-yl)-6- chloro-2-(((S)-1-methylpyrrolidin- 2-yl)methoxy)pyrido[2,3- d]pyrimidin-7-yl)naphthalen-2-ol | 531.2 |
| P-0046 | 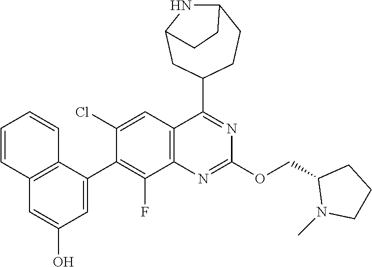 [SMILES: CN1CCC[C@H]1COc1nc(C2CCC3CCC(C2)N3)c2cc(Cl)c(-c3cc(O)cc4ccccc34)c(F)c2n1] | 4-(4-(3,9-diazabicyclo[4.2.1]nonan- 3-yl)-6-chloro-8-fluoro-2-(((S)-1- methylpyrrolidin-2- yl)methoxy)quinazolin-7- yl)naphthalen-2-ol | 562.1 |
| P-0047 | 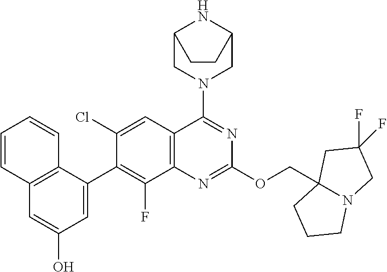 [SMILES: Oc1cc(-c2c(Cl)cc3c(N4CC5CCC(C4)N5)nc(OCC45CCCN4CC(F)(F)C5)nc3c2F)c2ccccc2c1] | 4-(4-((1R,5S)-3,8- diazabicyclo[3.2.1]octan-3-yl)-6- chloro-2-((2,2-difluorotetrahydro- 1H-pyrrolizin-7a(5H)-yl)methoxy)- 8-fluoroquinazolin-7-yl)naphthalen- 2-ol | 610.3 |
| P-0048 | 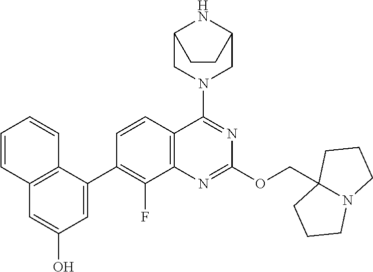 [SMILES: Oc1cc(-c2ccc3c(N4CC5CCC(C4)N5)nc(OCC45CCCN4CCC5)nc3c2F)c2ccccc2c1] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7- yl)naphthalen-2-ol | 540.2 |
| P-0049 | 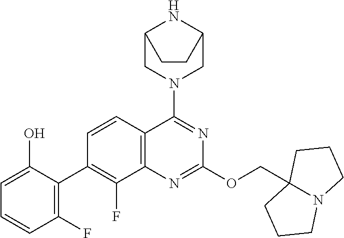 [SMILES: Oc1cccc(F)c1-c1ccc2c(N3CC4CCC(C3)N4)nc(OCC34CCCN3CCC4)nc2c1F] | 2-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7- yl)-3-fluorophenol | 508.2 |
| P-0050 | 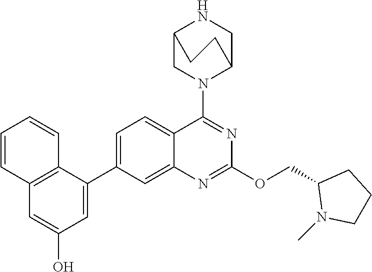 [SMILES: CN1CCC[C@H]1COc1nc(N2CC3CCC2CN3)c2ccc(-c3cc(O)cc4ccccc34)cc2n1] | 4-(4-(2,5-diazabicyclo[2.2.2]octan- 2-yl)-2-(((S)-1-methylpyrrolidin-2- yl)methoxy)quinazolin-7- yl)naphthalen-2-ol | 496.1 |
| P-0051 | 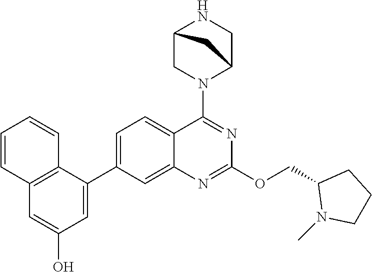 [SMILES: CN1CCC[C@H]1COc1nc(N2C[C@H]3C[C@@H]2CN3)c2ccc(-c3cc(O)cc4ccccc34)cc2n1] | 4-(4-((1R,4R)-2,5- diazabicyclo[2.2.1]heptan-2-yl)-2- (((S)-1-methylpyrrolidin-2- yl)methoxy)quinazolin-7- yl)naphthalen-2-ol | 482.1 |
| P-0052 | 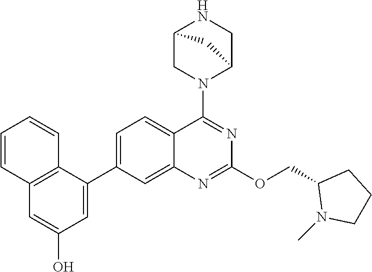 [SMILES: CN1CCC[C@H]1COc1nc(N2C[C@@H]3C[C@H]2CN3)c2ccc(-c3cc(O)cc4ccccc34)cc2n1] | 4-(4-((1S,4S)-2,5- diazabicyclo[2.2.1]heptan-2-yl)-2- (((S)-1-methylpyrrolidin-2- yl)methoxy)quinazolin-7- yl)naphthalen-2-ol | 482.1 |
| P-0053 | 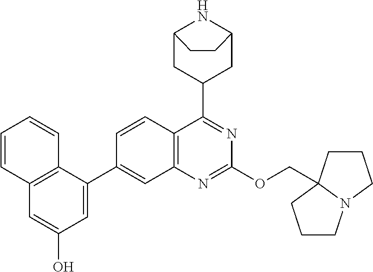 [SMILES: Oc1cc(-c2ccc3c(C4CC5CCC(C4)N5)nc(OCC45CCCN4CCC5)nc3c2)c2ccccc2c1] | 4-(4-((1R,5S)-8- azabicyclo[3.2.1]octan-3-yl)-2- ((tetrahydro-1H-pyrrolizin-7a(5H)- yl)methoxy)quinazolin-7- yl)naphthalen-2-ol | 521.3 |
| P-0054 | 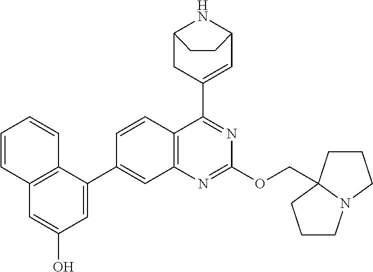 [SMILES: Oc1cc(-c2ccc3c(C4=CC5CCC(C4)N5)nc(OCC45CCCN4CCC5)nc3c2)c2ccccc2c1] | 4-(4-((1S,5R)-8- azabicyclo[3.2.1]oct-2-en-3-yl)-2- ((tetrahydro-1H-pyrrolizin-7a(5H)- yl)methoxy)quinazolin-7- yl)naphthalen-2-ol | 519.2 |
| P-0055 | 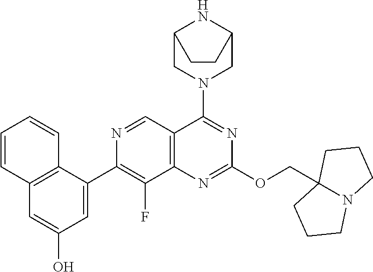 [SMILES: Oc1cc(-c2ncc3c(N4CC5CCC(C4)N5)nc(OCC45CCCN4CCC5)nc3c2F)c2ccccc2c1] | 4-(4-((1R,5S)-3,8- diazabicyclo[3.2.1]octan-3-yl)-8- fluoro-2-((tetrahydro-1H-pyrrolizin- 7a(5H)-yl)methoxy)pyrido[4,3- d]pyrimidin-7-yl)naphthalen-2-ol | 541.3 |
| P-0056 | 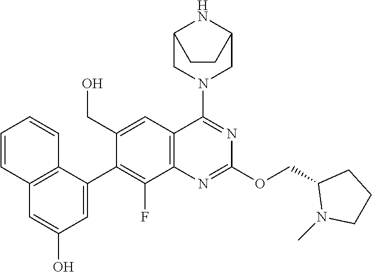 [SMILES: CN1CCC[C@H]1COc1nc(N2CC3CCC(C2)N3)c2cc(CO)c(-c3cc(O)cc4ccccc34)c(F)c2n1] | 4-(4-((1R,5S)-3,8- diazabicyclo[3.2.1]octan-3-yl)-8- fluoro-6-(hydroxymethyl)-2-(((S)- 1-methylpyrrolidin-2- yl)methoxy)quinazolin-7- yl)naphthalen-2-ol | 544.3 |
| P-0057 | 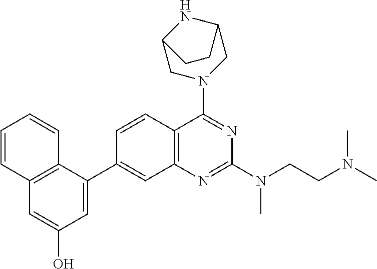 [SMILES: CN(C)CCN(C)c1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- ((2- (dimethylamino)ethyl)(methyl)amino) quinazolin-7-yl)naphthalen-2-ol | 483.3 |
| P-0058 | 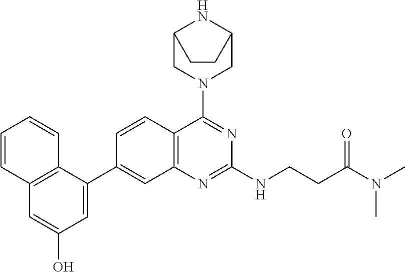 [SMILES: CN(C)C(=O)CCNc1nc(N2CC3CCC(C2)N3)c2ccc(-c3cc(O)cc4ccccc34)cc2n1] | 3-((4-((1R,5S)-3,8- diazabicyclo[3.2.1]octan-3-yl)-7-(3- hydroxynaphthalen-1-yl)quinazolin- 2-yl)amino)-N,N- dimethylpropanamide | 497.6 |
| P-0059 | 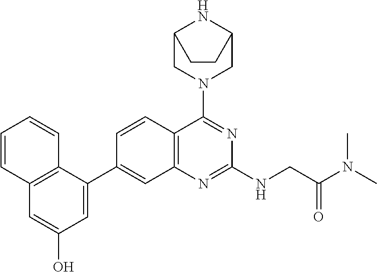 [SMILES: CN(C)C(=O)CNc1nc(N2CC3CCC(C2)N3)c2ccc(-c3cc(O)cc4ccccc34)cc2n1] | 2-((4-((1R,5S)-3,8- diazabicyclo[3.2.1]octan-3-yl)-7-(3- hydroxynaphthalen-1-yl)quinazolin- 2-yl)amino)-N,N- dimethylacetamide | 483.6 |
| P-0060 | 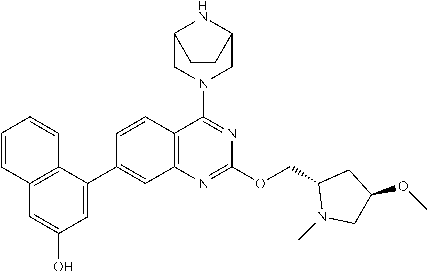 [SMILES: CO[C@@H]1C[C@@H](COc2nc(N3CC4CCC(C3)N4)c3ccc(-c4cc(O)cc5ccccc45)cc3n2)N(C)C1] | 4-(4-((1R,5S)-3,8- diazabicyclo[3.2.1]octan-3-yl)-2- (((2S,4R)-4-methoxy-1- methylpyrrolidin-2- yl)methoxy)quinazolin-7- yl)naphthalen-2-ol | 526.2 |
| P-0061 | 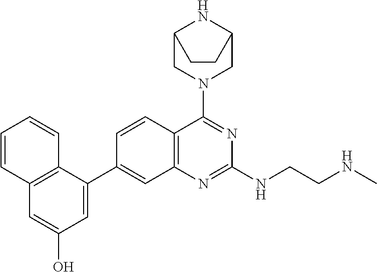 [SMILES: CNCCNc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- ((2- (methylamino)ethyl)amino)quinazo- lin-7-yl)naphthalen-2-ol | 455.2 |
| P-0062 | 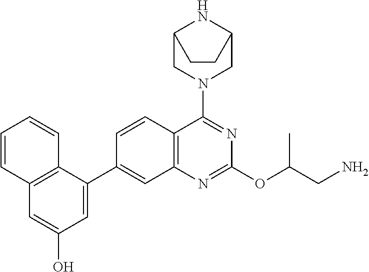 [SMILES: CC(CN)Oc1nc(N2CC3CCC(C2)N3)c2ccc(-c3cc(O)cc4ccccc34)cc2n1] | 4-(2-((1-aminopropan-2-yl)oxy)-4- ((1R,5S)-3,8- diazabicyclo[3.2.1]octan-3- yl)quinazolin-7-yl)naphthalen-2-ol | 456.2 |
| P-0063 | 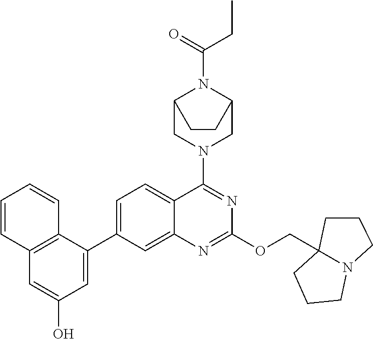 [SMILES: CCC(=O)N1C2CCC1CN(c1nc(OCC34CCCN3CCC4)nc3cc(-c4cc(O)cc5ccccc45)ccc13)C2] | 1-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)propan-1-one | 578.7 |
| P-0064 | 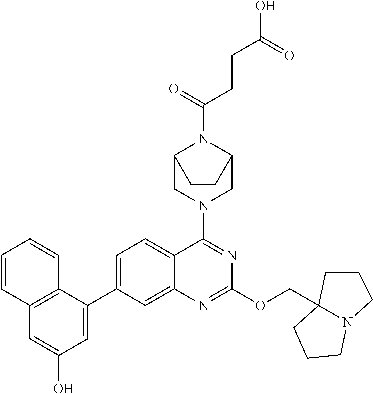 [SMILES: O=C(O)CCC(=O)N1C2CCC1CN(c1nc(OCC34CCCN3CCC4)nc3cc(-c4cc(O)cc5ccccc45)ccc13)C2] | 4-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-4- oxobutanoic acid | 622.7 |
| P-0065 | 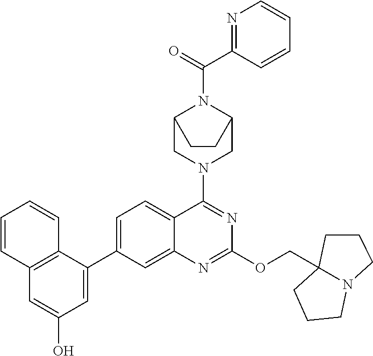 [SMILES: O=C(c1ccccn1)N1C2CCC1CN(c1nc(OCC34CCCN3CCC4)nc3cc(-c4cc(O)cc5ccccc45)ccc13)C2] | ((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)(pyridin-2-yl)methanone | 627.7 |
| P-0066 | 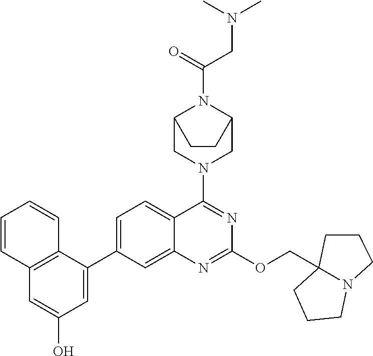 [SMILES: CN(C)CC(=O)N1C2CCC1CN(c1nc(OCC34CCCN3CCC4)nc3cc(-c4cc(O)cc5ccccc45)ccc13)C2] | 2-(dimethylamino)-1-((1R,5S)-3-(7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethan-1-one | 607.7 |
| P-0067 | 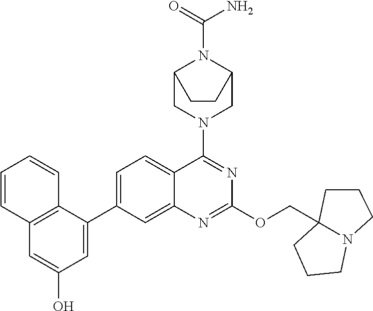 [SMILES: NC(=O)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 565.7 |
| P-0068 | 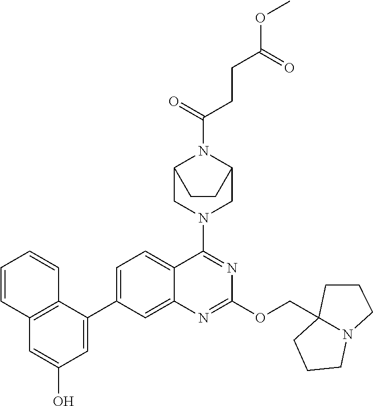 [SMILES: COC(=O)CCC(=O)N1C2CCC1CN(c1nc(OCC34CCCN3CCC4)nc3cc(-c4cc(O)cc5ccccc45)ccc13)C2] | methyl 4-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1 ]octan-8-yl)-4- oxobutanoate | 634.2 |
| P-0069 | 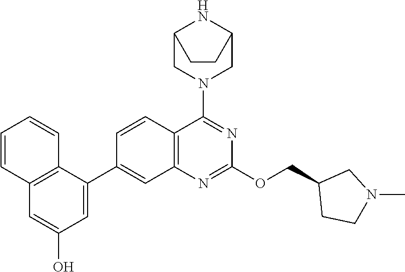 [SMILES: CN1CC[C@@H](COc2nc(N3CC4CCC(C3)N4)c3ccc(-c4cc(O)cc5ccccc45)cc3n2)C1] | 4-(4-((1R,5S)-3,8- diazabicyclo[3.2.1]octan-3-yl)-2- (((R)-1-methylpyrrolidin-3- yl)methoxy)quinazolin-7- yl)naphthalen-2-ol | 496.20 |
| P-0070 | 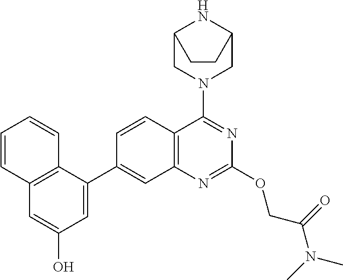 [SMILES: CN(C)C(=O)COc1nc(N2CC3CCC(C2)N3)c2ccc(-c3cc(O)cc4ccccc34)cc2n1] | 2-((4-((1R,5S)-3,8- diazabicyclo[3.2.1]octan-3-yl)-7-(3- hydroxynaphthalen-1-yl)quinazolin- 2-yl)oxy)-N,N-dimethylacetamide | 484.55 |
| P-0071 | 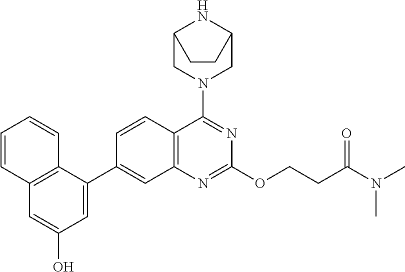 [SMILES: CN(C)C(=O)CCOc1nc(N2CC3CCC(C2)N3)c2ccc(-c3cc(O)cc4ccccc34)cc2n1] | 3-((4-((1R,5S)-3,8- diazabicyclo[3.2.1]octan-3-yl)-7-(3- hydroxynaphthalen-1-yl)quinazolin- 2-yl)oxy)-N,N- dimethylpropanamide | 498.20 |
| P-0072 | 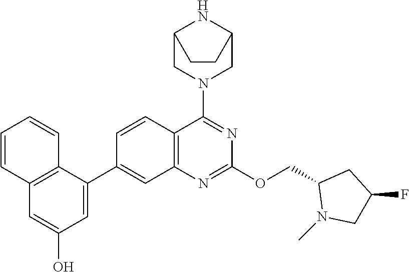 [SMILES: CN1C[C@H](F)C[C@H]1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- (((2S,4R)-4-fluoro-1- methylpyrrolidin-2- yl)methoxy)quinazolin-7- yl)naphthalen-2-ol | 514.20 |
| P-0073 | 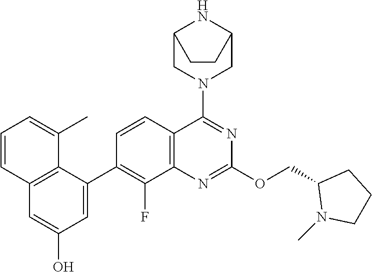 [SMILES: Cc1cccc2cc(O)cc(-c3ccc4c(N5CC6CCC(C5)N6)nc(OC[C@@H]5CCCN5C)nc4c3F)c12] | 4-(4-((1R,5S)-3,8- diazabicyclo[3.2.1]octan-3-yl)-8- fluoro-2-(((S)-1-methylpyrrolidin- 2-yl)methoxy)quinazolin-7-yl)-5- methylnaphthalen-2-ol | 528.2 |
| P-0074 | 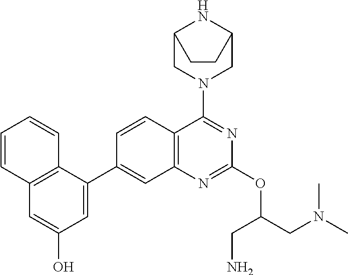 [SMILES: CN(C)CC(CN)Oc1nc(N2CC3CCC(C2)N3)c2ccc(-c3cc(O)cc4ccccc34)cc2n1] | 4-(2-((1-amino-3- (dimethylamino)propan-2-yl)oxy)- 4-((1R,5S)-3,8- diazabicyclo[3.2.1]octan-3- yl)quinazolin-7-yl)naphthalen-2-ol | 499.1 |
| P-0075 | 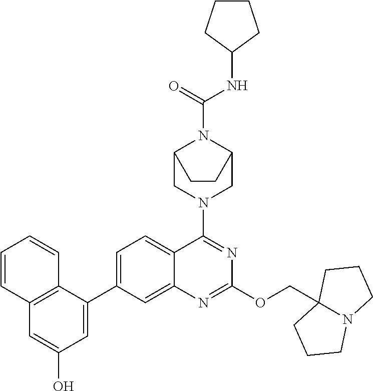 [SMILES: O=C(NC1CCCC1)N1C2CCC1CN(c1nc(OCC34CCCN3CCC4)nc3cc(-c4cc(O)cc5ccccc45)ccc13)C2] | (1R,5S)-N-cyclopentyl-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 633.3 |
| P-0076 | 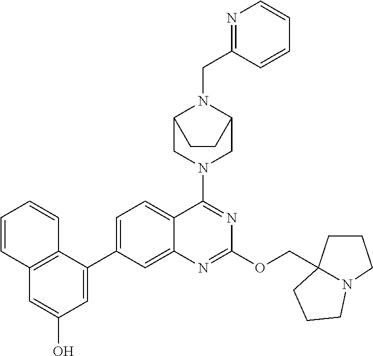 [SMILES: Oc1cc(-c2ccc3c(N4CC5CCC(C4)N5Cc4ccccn4)nc(OCC45CCCN4CCC5)nc3c2)c2ccccc2c1] | 4-(4-((1R,5S)-8-(pyridin-2- ylmethyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 612.3 |
| P-0077 | 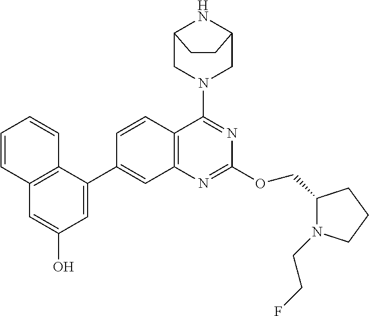 [SMILES: Oc1cc(-c2ccc3c(N4CC5CCC(C4)N5)nc(OC[C@@H]4CCCN4CCF)nc3c2)c2ccccc2c1] | 4-(4-((1R,5S)-3,8- diazabicyclo[3.2.1]octan-3-yl)-2- (((S)-1-(2-fluoroethyl)pyrrolidin-2- yl)methoxy)quinazolin-7- yl)naphthalen-2-ol | 528.1 |
| P-0078 | 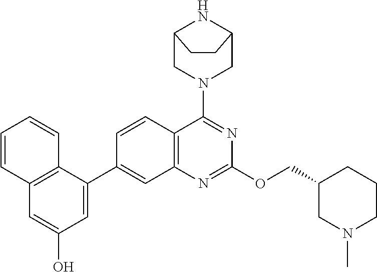 [SMILES: CN1CCC[C@@H](COc2nc(N3CC4CCC(C3)N4)c3ccc(-c4cc(O)cc5ccccc45)cc3n2)C1] | 4-(4-((1R,5S)-3,8- diazabicyclo[3.2.1]octan-3-yl)-2- (((R)-1-methylpiperidin-3- yl)methoxy)quinazolin-7- yl)naphthalen-2-ol | 510.1 |
| P-0079 | 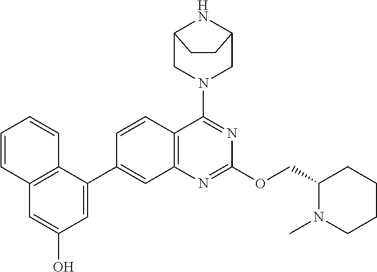 [SMILES: CN1CCCC[C@H]1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- (((S)-1-methylpiperidin-2- yl)methoxy)quinazolin-7- yl)naphthalen-2-ol | 510.2 |
| P-0080 | 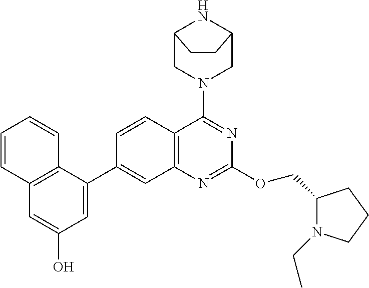 [SMILES: CCN1CCC[C@H]1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- (((S)-1-ethylpyrrolidin-2- yl)methoxy)quinazolin-7- yl)naphthalen-2-ol | 510.1 |
| P-0081 | 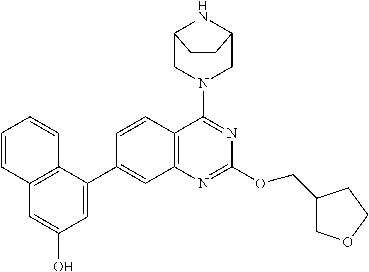 [SMILES: Oc1cc(-c2ccc3c(N4CC5CCC(C4)N5)nc(OCC4CCOC4)nc3c2)c2ccccc2c1] | 4-(4-((1R,5S)-3,8- diazabicyclo[3.2.1]octan-3-yl)-2- ((tetrahydrofuran-3- yl)methoxy)quinazolin-7- yl)naphthalen-2-ol | 483.1 |
| P-0082 | 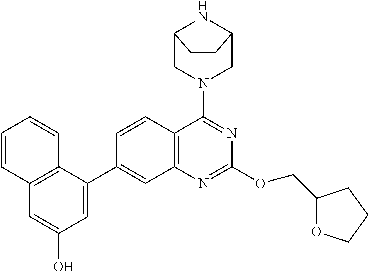 [SMILES: Oc1cc(-c2ccc3c(N4CC5CCC(C4)N5)nc(OCC4CCCO4)nc3c2)c2ccccc2c1] | 4-(4-((1R,5S)-3,8- diazabicyclo[3.2.1]octan-3-yl)-2- ((tetrahydrofuran-2- yl)methoxy)quinazolin-7- yl)naphthalen-2-ol | 483.1 |
| P-0083 | 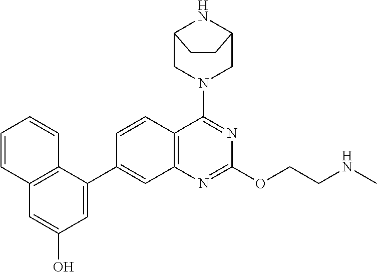 [SMILES: CNCCOc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2-(2- (methylamino)ethoxy)quinazolin-7- yl)naphthalen-2-ol | 456.2 |
| P-0084 | 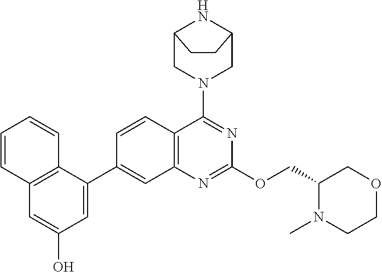 [SMILES: CN1CCOC[C@H]1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- (((S)-4-methylmorpholin-3- yl)methoxy)quinazolin-7- yl)naphthalen-2-ol | 512.2 |
| P-0085 | 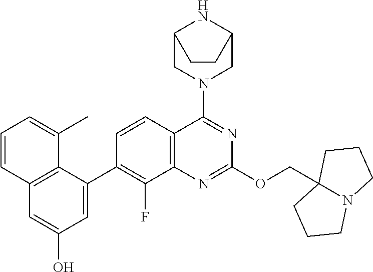 [SMILES: Cc1cccc2cc(O)cc(-c3ccc4c(N5CC6CCC(C5)N6)nc(OCC56CCCN5CCC6)nc4c3F)c12] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7- yl)-5-methylnaphthalen-2-ol | 554.3 |
| P-0086 | 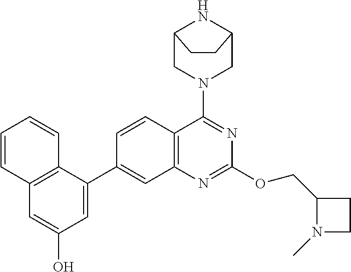 [SMILES: CN1CCC1COc1nc(N2CC3CCC(C2)N3)c2ccc(-c3cc(O)cc4ccccc34)cc2n1] | 4-(4-((1R,5S)-3,8- diazabicyclo[3.2.1]octan-3-yl)-2- ((1-methylazetidin-2- yl)methoxy)quinazolin-7- yl)naphthalen-2-ol | 482.2 |
| P-0087 | 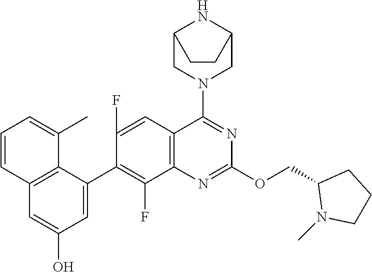 [SMILES: Cc1cccc2cc(O)cc(-c3c(F)cc4c(N5CC6CCC(C5)N6)nc(OC[C@@H]5CCCN5C)nc4c3F)c12] | 4-(4-((1R,5S)-3,8- diazabicyclo[3.2.1]octan-3-yl)-6,8- difluoro-2-(((S)-1-methylpyrrolidin- 2-yl)methoxy)quinazolin-7-yl)-5- methylnaphthalen-2-ol | 546.3 |
| P-0088 | 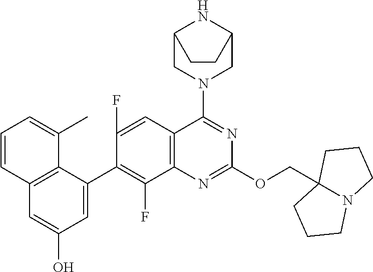 [SMILES: Cc1cccc2cc(O)cc(-c3c(F)cc4c(N5CC6CCC(C5)N6)nc(OCC56CCCN5CCC6)nc4c3F)c12] | 4-(4-((1R,5S)-3,8- diazabicyclo[3.2.1]octan-3-yl)-6,8- difluoro-2-((tetrahydro-1H- pyrrolizin-7a(5H)- yl)methoxy)quinazolin-7-yl)-5- methylnaphthalen-2-ol | 572.2 |
| P-0089 | 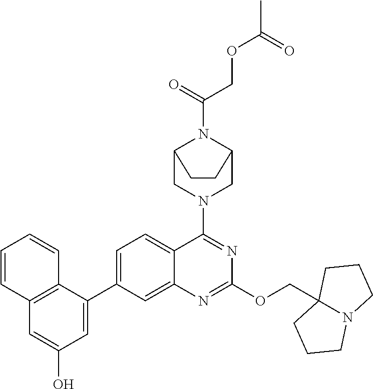 [SMILES: CC(=O)OCC(=O)N1C2CCC1CN(c1nc(OCC34CCCN3CCC4)nc3cc(-c4cc(O)cc5ccccc45)ccc13)C2] | 2-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl acetate | 620.2 |
| P-0090 | 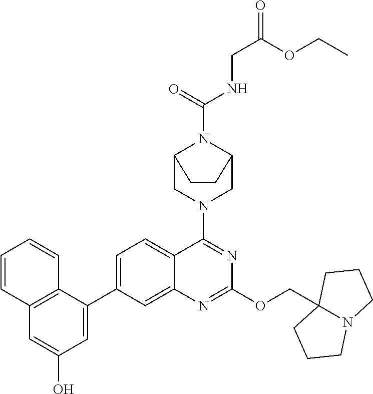 [SMILES: CCOC(=O)CNC(=O)N1C2CCC1CN(c1nc(OCC34CCCN3CCC4)nc3cc(-c4cc(O)cc5ccccc45)ccc13)C2] | ethyl ((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)glycinate | 651.3 |
| P-0091 | 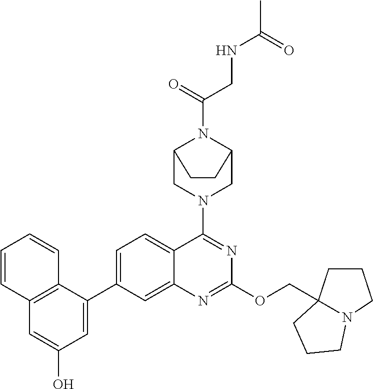 [SMILES: CC(=O)NCC(=O)N1C2CCC1CN(c1nc(OCC34CCCN3CCC4)nc3cc(-c4cc(O)cc5ccccc45)ccc13)C2] | N-(2-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)acetamide | 621.3 |
| P-0092 | 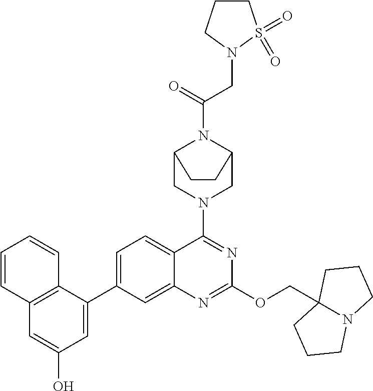 [SMILES: O=C(CN1CCCS1(=O)=O)N1C2CCC1CN(c1nc(OCC34CCCN3CCC4)nc3cc(-c4cc(O)cc5ccccc45)ccc13)C2] | 2-(1,1-dioxidoisothiazolidin-2-yl)- 1-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethan-1-one | 683.7 |
| P-0093 | 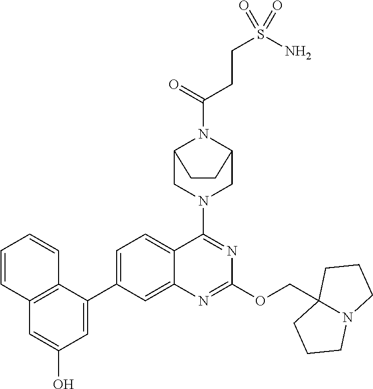 [SMILES: NS(=O)(=O)CCC(=O)N1C2CCC1CN(c1nc(OCC34CCCN3CCC4)nc3cc(-c4cc(O)cc5ccccc45)ccc13)C2] | 3-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropane-1-sulfonamide | 657.2 |
| P-0094 | 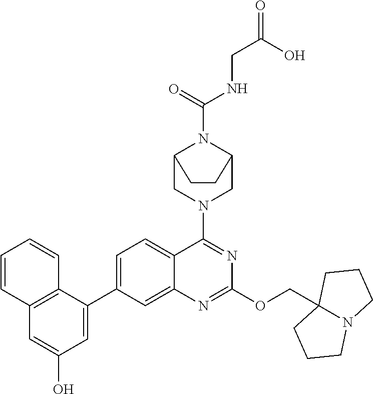 [SMILES: O=C(O)CNC(=O)N1C2CCC1CN(c1nc(OCC34CCCN3CCC4)nc3cc(-c4cc(O)cc5ccccc45)ccc13)C2] | ((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)glycine | 623.3 |
| P-0095 | 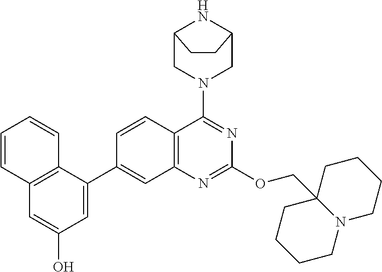 [SMILES: Oc1cc(-c2ccc3c(N4CC5CCC(C4)N5)nc(OCC45CCCCN4CCCC5)nc3c2)c2ccccc2c1] | 4-(4-((1R,5S)-3,8- diazabicyclo[3.2.1]octan-3-yl)-2- ((octahydro-9aH-quinolizin-9a- yl)methoxy)quinazolin-7- yl)naphthalen-2-ol | 550.3 |
| P-0096 | 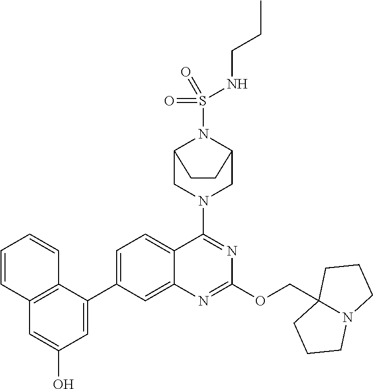 [SMILES: CCCNS(=O)(=O)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- propyl-3,8- diazabicyclo[3.2.1]octane-8- sulfonamide | 643.3 |
| P-0097 | 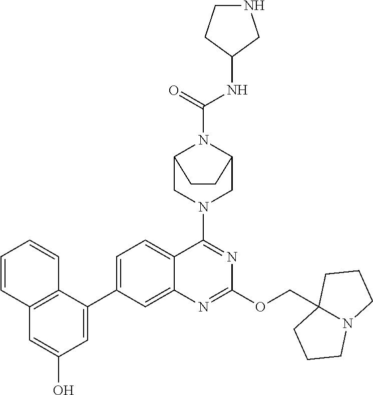 [SMILES: O=C(NC1CCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- (pyrrolidin-3-yl)-3,8- diazabicyclo[3.2.1]octane-8- carboxamide | 632.2 |
| P-0098 | 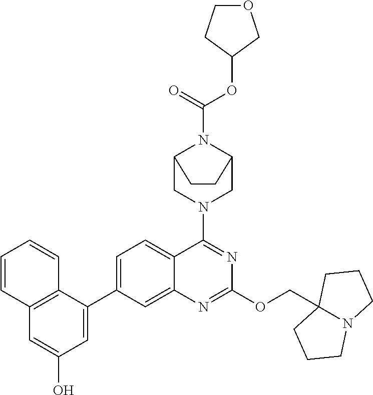 [SMILES: O=C(OC1CCOC1)N1C2CCC1CN(c1nc(OCC34CCCN3CCC4)nc3cc(-c4cc(O)cc5ccccc45)ccc13)C2] | tetrahydrofuran-3-yl (1R,5S)-3-(7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxylate | 636.3 |
| P-0099 | 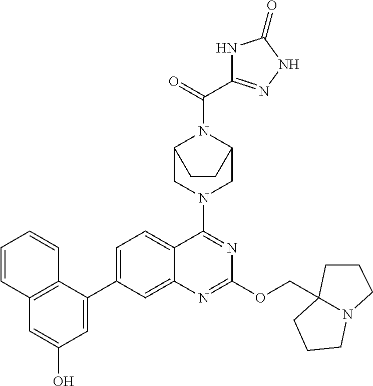 [SMILES: O=C(c1n[nH]c(=O)[nH]1)N1C2CCC1CN(c1nc(OCC34CCCN3CCC4)nc3cc(-c4cc(O)cc5ccccc45)ccc13)C2] | 5-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)-2,4-dihydro-3H-1,2,4- triazol-3-one | 633.7 |
| P-0100 | 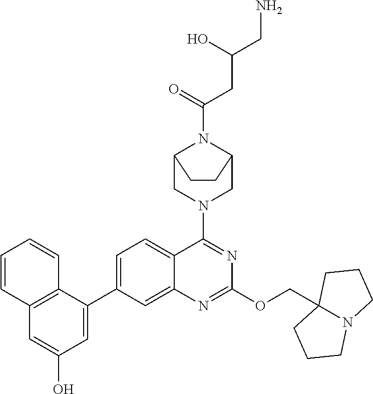 [SMILES: NCC(O)CC(=O)N1C2CCC1CN(c1nc(OCC34CCCN3CCC4)nc3cc(-c4cc(O)cc5ccccc45)ccc13)C2] | 4-amino-3-hydroxy-1-((1R,5S)-3- (7-(3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)butan-1-one | 623.3 |
| P-0101 | 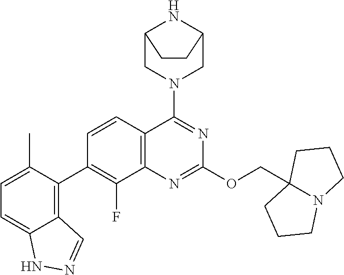 [SMILES: Cc1ccc2[nH]ncc2c1-c1ccc2c(N3CC4CCC(C3)N4)nc(OCC34CCCN3CCC4)nc2c1F] | 4-((1R,5S)-3,8- diazabicyclo[3.2.1]octan-3-yl)-8- fluoro-7-(5-methyl-1H-indazol-4- yl)-2-((tetrahydro-1H-pyrrolizin- 7a(5H)-yl)methoxy)quinazoline | 528.3 |
| P-0102 | 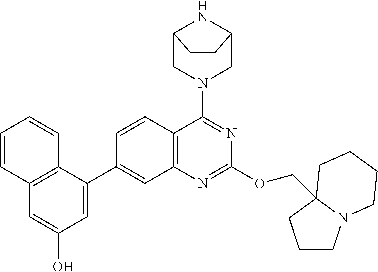 [SMILES: Oc1cc(-c2ccc3c(N4CC5CCC(C4)N5)nc(OCC45CCCCN4CCC5)nc3c2)c2ccccc2c1] | 4-(4-((1R,5S)-3,8- diazabicyclo[3.2.1]octan-3-yl)-2- ((hexahydroindolizin-8a(1H)- yl)methoxy)quinazolin-7- yl)naphthalen-2-ol | 536.2 |
| P-0103 | 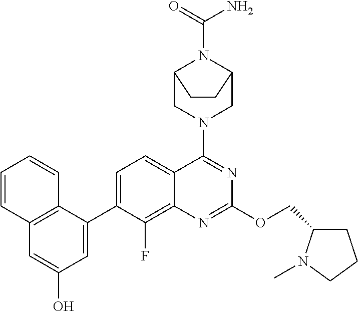 [SMILES: CN1CCC[C@H]1COc1nc(N2CC3CCC(C2)N3C(N)=O)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- carboxamide | 557.1 |
| P-0104 | 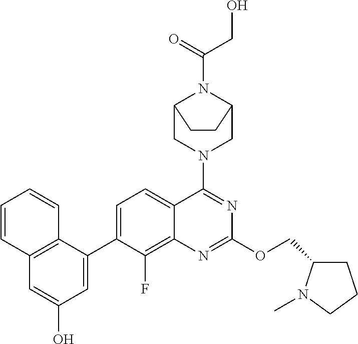 [SMILES: CN1CCC[C@H]1COc1nc(N2CC3CCC(C2)N3C(=O)CO)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- hydroxyethan-1-one | 572.1 |
| P-0105 | 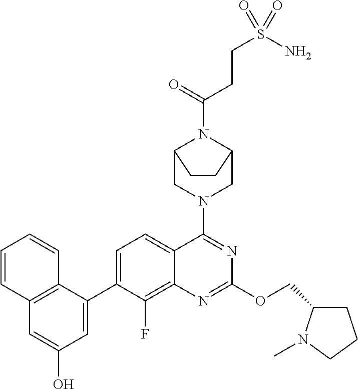 [SMILES: CN1CCC[C@H]1COc1nc(N2CC3CCC(C2)N3C(=O)CCS(N)(=O)=O)c2ccc(-c3cc(O)cc4ccccc34)c(F)c2n1] | 3-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- oxopropane-1-sulfonamide | 649.1 |
| P-0106 | 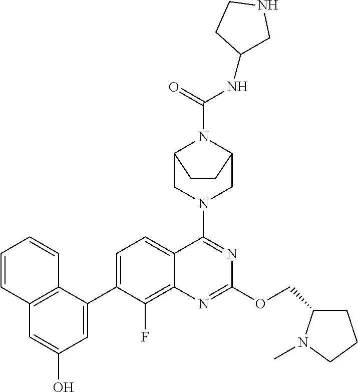 [SMILES: CN1CCC[C@H]1COc1nc(N2CC3CCC(C2)N3C(=O)NC2CCNC2)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-N- (pyrrolidin-3-yl)-3,8- diazabicyclo[3.2.1]octane-8- carboxamide | 626.2 |
| P-0107 | 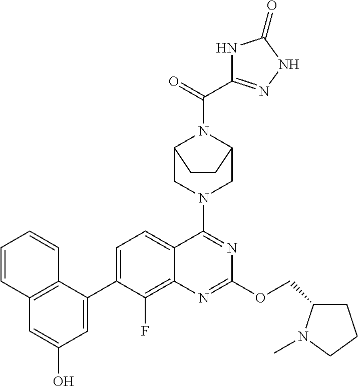 [SMILES: CN1CCC[C@H]1COc1nc(N2CC3CCC(C2)N3C(=O)c2n[nH]c(=O)[nH]2)c2ccc(-c3cc(O)cc4ccccc34)c(F)c2n1] | 5-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- carbonyl)-2,4-dihydro-3H-1,2,4- triazol-3-one | 626.2 |
| P-0108 | 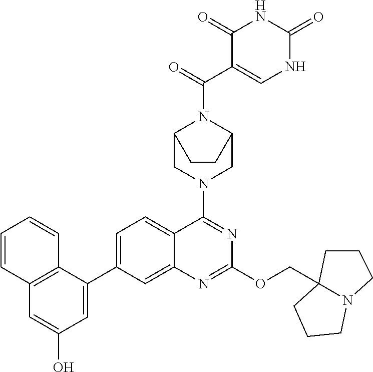 [SMILES: O=C(c1c[nH]c(=O)[nH]c1=O)N1C2CCC1CN(c1nc(OCC34CCCN3CCC4)nc3cc(-c4cc(O)cc5ccccc45)ccc13)C2] | 5-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)pyrimidine-2,4(1H,3H)- dione | 660.3 |
| P-0109 | 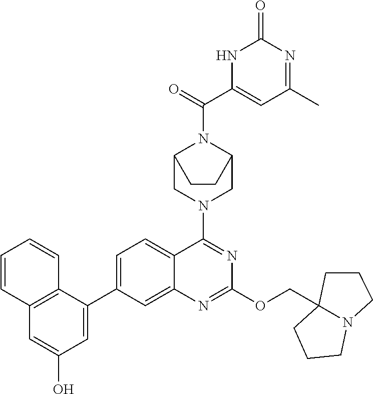 [SMILES: Cc1cc(C(=O)N2C3CCC2CN(c2nc(OCC45CCCN4CCC5)nc4cc(-c5cc(O)cc6ccccc56)ccc24)C3)[nH]c(=O)n1] | 6-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)-4-methylpyrimidin- 2(1H)-one | 658.7 |
| P-0110- | 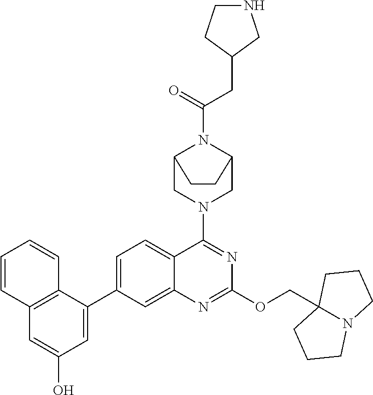 [SMILES: O=C(CC1CCNC1)N1C2CCC1CN(c1nc(OCC34CCCN3CCC4)nc3cc(-c4cc(O)cc5ccccc45)ccc13)C2] | 1-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- (pyrrolidin-3-yl)ethan-1-one | 633.30 |
| P-0111 | 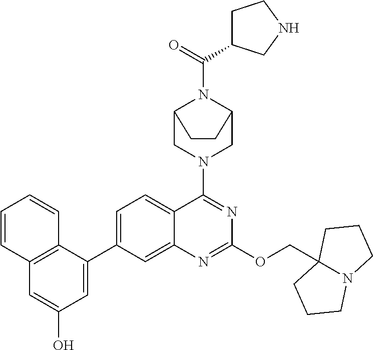 [SMILES: O=C([C@@H]1CCNC1)N1C2CCC1CN(c1nc(OCC34CCCN3CCC4)nc3cc(-c4cc(O)cc5ccccc45)ccc13)C2] | ((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)((R)- pyrrolidin-3-yl)methanone | 619.25 |
| P-0112 | 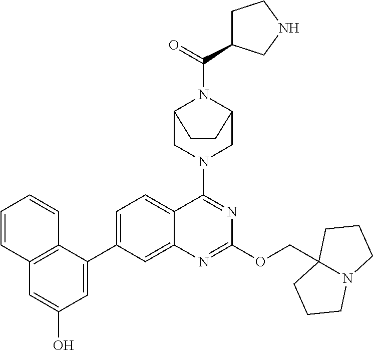 [SMILES: O=C([C@H]1CCNC1)N1C2CCC1CN(c1nc(OCC34CCCN3CCC4)nc3cc(-c4cc(O)cc5ccccc45)ccc13)C2] | ((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)((S)- pyrrolidin-3-yl)methanone | 619.30 |
| P-0113 | 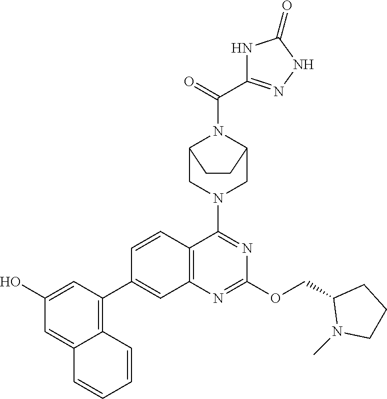 [SMILES: CN1CCC[C@H]1COc1nc(N2CC3CCC(C2)N3C(=O)c2n[nH]c(=O)[nH]2)c2ccc(-c3cc(O)cc4ccccc34)cc2n1] | 5-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octane-8- carbonyl)-2,4-dihydro-3H-1,2,4- triazol-3-one | 607.1 |
| P-0114 | 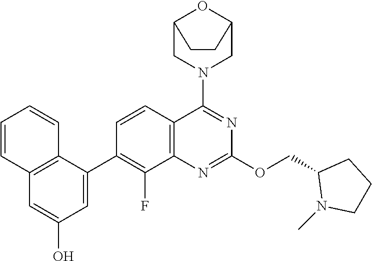 [SMILES: CN1CCC[C@H]1COc1nc(N2CC3CCC(C2)O3)c2ccc(-c3cc(O)cc4ccccc34)c(F)c2n1] | 4-(4-((1R,5S)-8-oxa-3- azabicyclo[3.2.1]octan-3-yl)-8- fluoro-2-(((S)-1-methylpyrrolidin- 2-yl)methoxy)quinazolin-7- yl)naphthalen-2-ol | 515.05 |
| P-0115 | 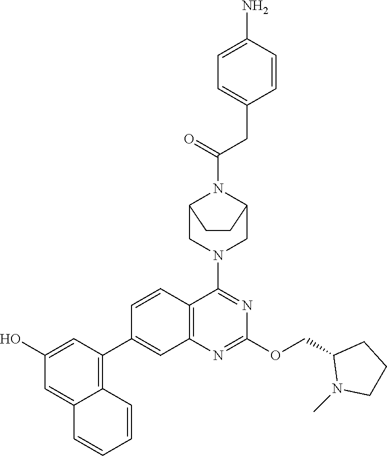 [SMILES: CN1CCC[C@H]1COc1nc(N2CC3CCC(C2)N3C(=O)Cc2ccc(N)cc2)c2ccc(-c3cc(O)cc4ccccc34)cc2n1] | 2-(4-aminophenyl)-1-((1R,5S)-3-(7- (3-hydroxynaphthalen-1-yl)-2-(((S)- 1-methylpyrrolidin-2- yl)methoxy)quinazolin-4-yl)-3,8- diazabicyclo[3.2.1]octan-8- yl)ethan-1-one | 629.5 |
| P-0116 | 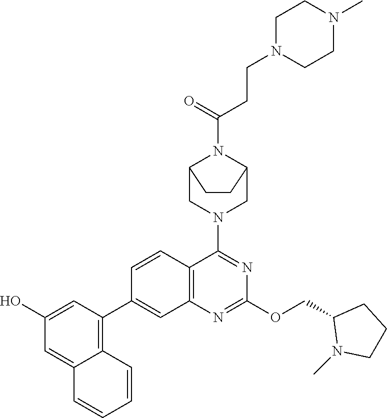 [SMILES: CN1CCN(CCC(=O)N2C3CCC2CN(c2nc(OC[C@@H]4CCCN4C)nc4cc(-c5cc(O)cc6ccccc56)ccc24)C3)CC1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3-(4- methylpiperazin-1-yl)propan-1-one | 650.4 |
| P-0117 | 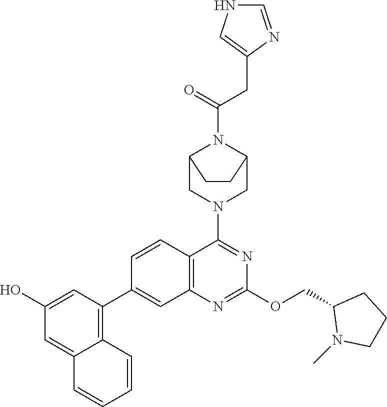 [SMILES: CN1CCC[C@H]1COc1nc(N2CC3CCC(C2)N3C(=O)Cc2c[nH]cn2)c2ccc(-c3cc(O)cc4ccccc34)cc2n1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (1H-imidazol-4-yl)ethan-1-one | 604.3 |
| P-0118 | 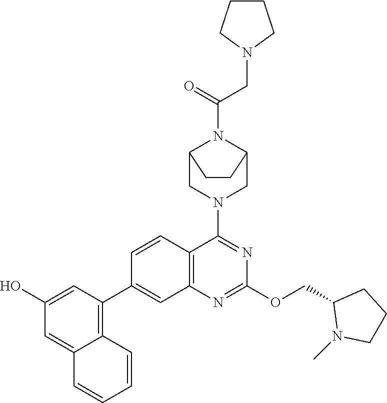 [SMILES: CN1CCC[C@H]1COc1nc(N2CC3CCC(C2)N3C(=O)CN2CCCC2)c2ccc(-c3cc(O)cc4ccccc34)cc2n1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (pyrrolidin-1-yl)ethan-1-one | 607.4 |
| P-0119 | 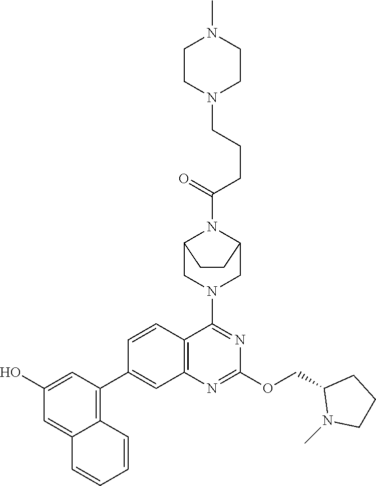 [SMILES: CN1CCN(CCCC(=O)N2C3CCC2CN(c2nc(OC[C@@H]4CCCN4C)nc4cc(-c5cc(O)cc6ccccc56)ccc24)C3)CC1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-4-(4- methylpiperazin-1-yl)butan-1-one | 664.4 |
| P-0120 | 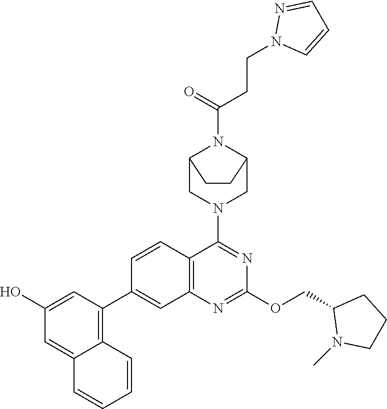 [SMILES: CN1CCC[C@H]1COc1nc(N2CC3CCC(C2)N3C(=O)CCn2cccn2)c2ccc(-c3cc(O)cc4ccccc34)cc2n1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- (1H-pyrazol-1-yl)propan-1-one | 618.4 |
| P-0121 | 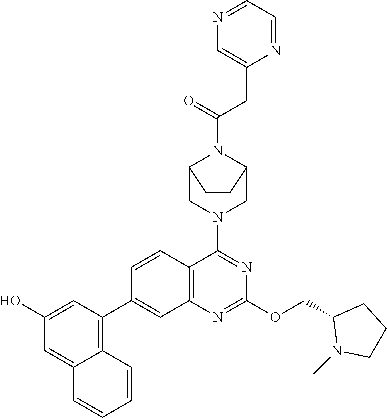 [SMILES: CN1CCC[C@H]1COc1nc(N2CC3CCC(C2)N3C(=O)Cc2cnccn2)c2ccc(-c3cc(O)cc4ccccc34)cc2n1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (pyrazin-2-yl)ethan-1-one | 616.4 |
| P-0122 | 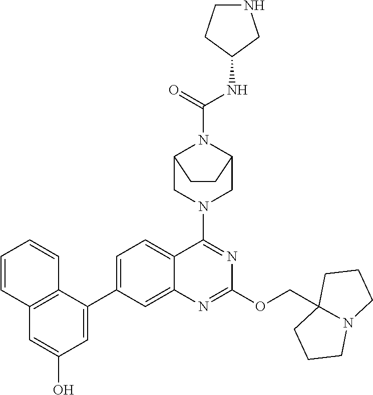 [SMILES: O=C(N[C@@H]1CCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- ((R)-pyrrolidin-3-yl)-3,8- diazabicyclo[3.2.1]octane-8- carboxamide | 634.3 |
| P-0123 | 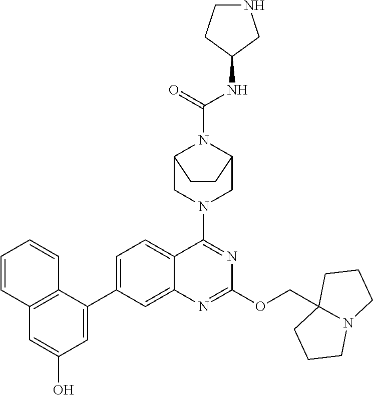 [SMILES: O=C(N[C@H]1CCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- ((S)-pyrrolidin-3-yl)-3,8- diazabicyclo[3.2.1]octane-8- carboxamide | 634.3 |
| P-0124 | 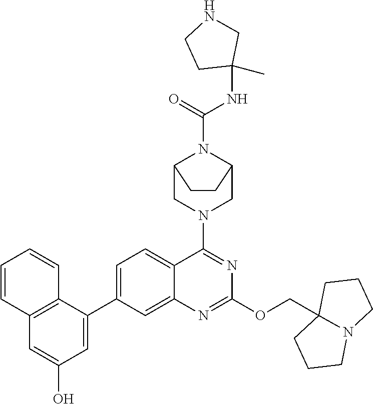 [SMILES: CC1(NC(=O)N2C3CCC2CN(c2nc(OCC45CCCN4CCC5)nc4cc(-c5cc(O)cc6ccccc56)ccc24)C3)CCNC1] | (1R,5S)-3-(7-(3- hydroxynaphthalen-1-yl)-2- ((tetrahydro-1H-pyrrolizin-7a(5H)- yl)methoxy)quinazolin-4-yl)-N-(3- methylpyrrolidin-3-yl)-3,8- diazabicyclo[3.2.1]octane-8- carboxamide | 648.2 |
| P-0125 | 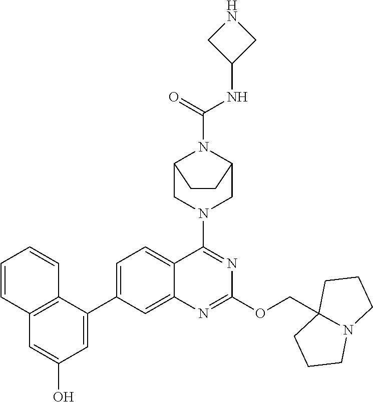 [SMILES: O=C(NC1CNC1)N1C2CCC1CN(c1nc(OCC34CCCN3CCC4)nc3cc(-c4cc(O)cc5ccccc45)ccc13)C2] | (1R,5S)-N-(azetidin-3-yl)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 620.3 |
| P-0126 | 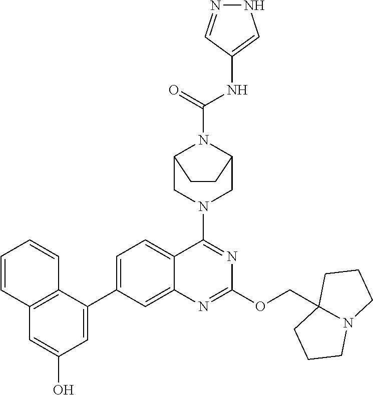 [SMILES: O=C(Nc1cn[nH]c1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- (1H-pyrazol-4-yl)-3,8- diazabicyclo[3.2.1]octane-8- carboxamide | 631.2 |
| P-0127 | 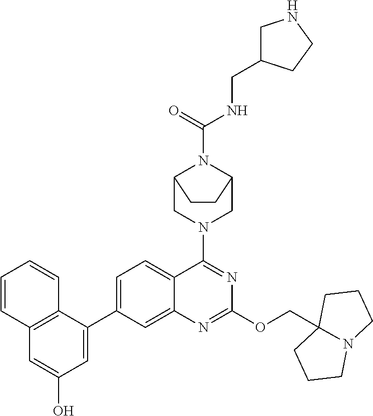 [SMILES: O=C(NCC1CCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- (pyrrolidin-3-ylmethyl)-3,8- diazabicyclo[3.2.1]octane-8- carboxamide | 646.2 |
| P-0128 | 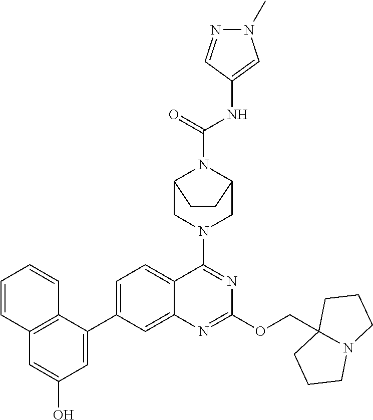 [SMILES: Cn1cc(NC(=O)N2C3CCC2CN(c2nc(OCC45CCCN4CCC5)nc4cc(-c5cc(O)cc6ccccc56)ccc24)C3)cn1] | (1R,5S)-3-(7-(3- hydroxynaphthalen-1-yl)-2- ((tetrahydro-1H-pyrrolizin-7a(5H)- yl)methoxy)quinazolin-4-yl)-N-(1- methyl-1H-pyrazol-4-yl)-3,8- diazabicyclo[3.2.1]octane-8- carboxamide | 645.3 |
| P-0129 | 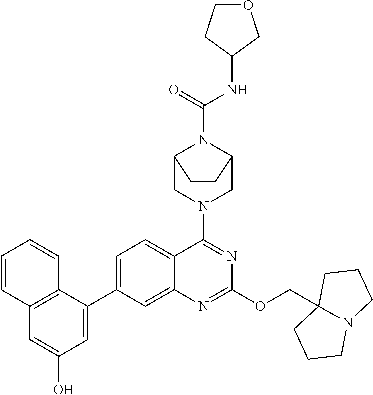 [SMILES: O=C(NC1CCOC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- (tetrahydrofuran-3-yl)-3,8- diazabicyclo[3.2.1]octane-8- carboxamide | 633.2 |
| P-0130 | 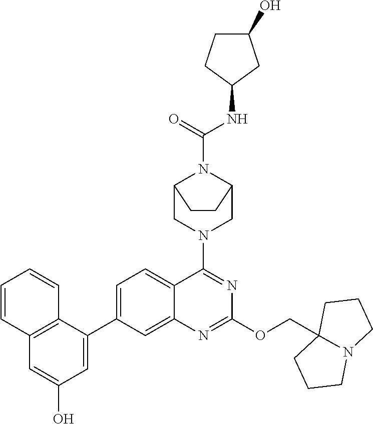 [SMILES: O=C(N[C@H]1CC[C@@H](O)C1)N1C2CCC1CN(c1nc(OCC34CCCN3CCC4)nc3cc(-c4cc(O)cc5ccccc45)ccc13)C2] | (1R,5S)-N-((1S,3R)-3- hydroxy cyclopentyl)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 649.7 |
| P-0131 | 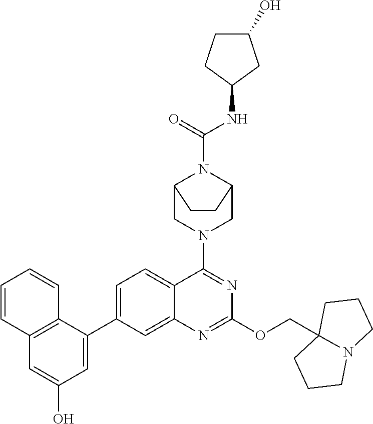 [SMILES: O=C(N[C@H]1CC[C@H](O)C1)N1C2CCC1CN(c1nc(OCC34CCCN3CCC4)nc3cc(-c4cc(O)cc5ccccc45)ccc13)C2] | (1R,5S)-N-((1S,3S)-3- hydroxycyclopentyl)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 649.3 |
| P-0132 | 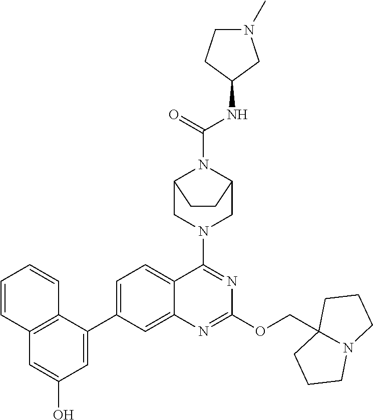 [SMILES: CN1CC[C@H](NC(=O)N2C3CCC2CN(c2nc(OCC45CCCN4CCC5)nc4cc(-c5cc(O)cc6ccccc56)ccc24)C3)C1] | (1R,5S)-3-(7-(3- hydroxynaphthalen-1-yl)-2- ((tetrahydro-1H-pyrrolizin-7a(5H)- yl)methoxy)quinazolin-4-yl)-N- ((S)-1-methylpyrrolidin-3-yl)-3,8- diazabicyclo[3.2.1]octane-8- carboxamide | 648.3 |
| P-0133 | 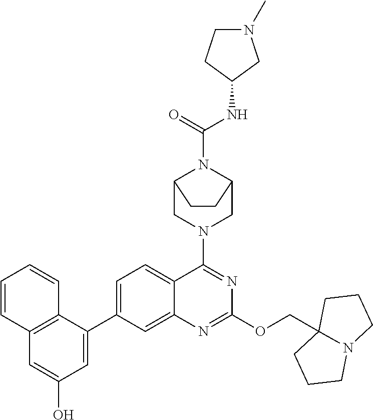 [SMILES: CN1CC[C@@H](NC(=O)N2C3CCC2CN(c2nc(OCC45CCCN4CCC5)nc4cc(-c5cc(O)cc6ccccc56)ccc24)C3)C1] | (1R,5S)-3-(7-(3- hydroxynaphthalen-1-yl)-2- ((tetrahydro-1H-pyrrolizin-7a(5H)- yl)methoxy)quinazolin-4-yl)-N- ((R)-1-methylpyrrolidin-3-yl)-3,8- diazabicyclo[3.2.1]octane-8- carboxamide | 648.3 |
| P-0134 | 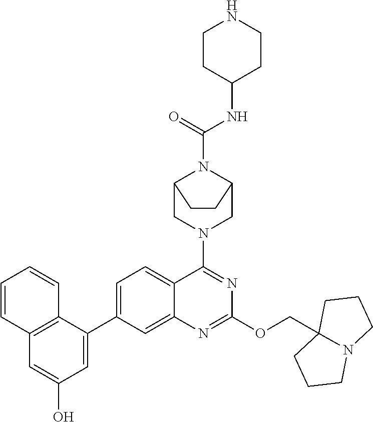 [SMILES: O=C(NC1CCNCC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- (piperidin-4-yl)-3,8- diazabicyclo[3.2.1]octane-8- carboxamide | 648.3 |
| P-0135 | 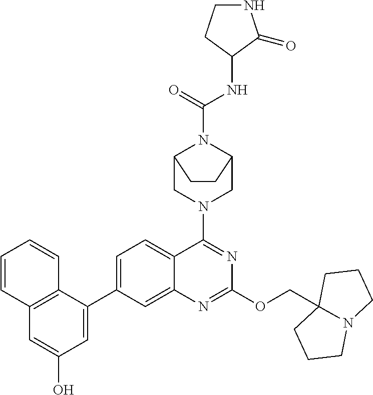 [SMILES: O=C1NCCC1NC(=O)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N-(2- oxopyrrolidin-3-yl)-3,8- diazabicyclo[3.2.1]octane-8- carboxamide | 648.2 |
| P-0136 | 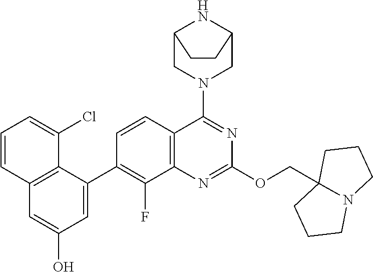 [SMILES: Oc1cc(-c2ccc3c(N4CC5CCC(C4)N5)nc(OCC45CCCN4CCC5)nc3c2F)c2c(Cl)cccc2c1] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7- yl)-5-chloronaphthalen-2-ol | 574.2 |
| P-0137 | 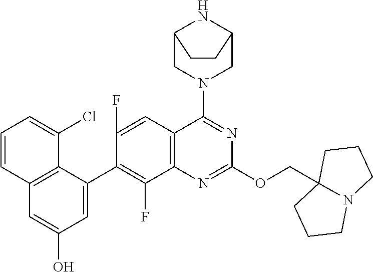 [SMILES: Oc1cc(-c2c(F)cc3c(N4CC5CCC(C4)N5)nc(OCC45CCCN4CCC5)nc3c2F)c2c(Cl)cccc2c1] | 4-(4-((1R,5S)-3,8- diazabicyclo[3.2.1]octan-3-yl)-6,8- difluoro-2-((tetrahydro-1H- pyrrolizin-7a(5H)- yl)methoxy)quinazolin-7-yl)-5- chloronaphthalen-2-ol | 592.3 |
| P-0138 | 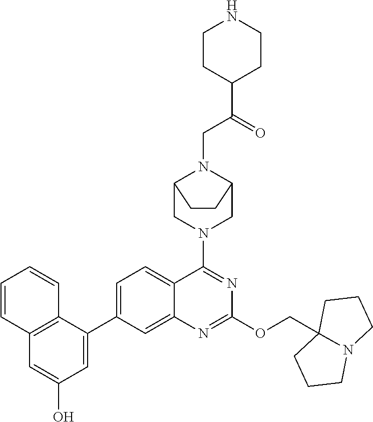 [SMILES: O=C(CN1C2CCC1CN(c1nc(OCC34CCCN3CCC4)nc3cc(-c4cc(O)cc5ccccc45)ccc13)C2)C1CCNCC1] | 2-((1R,5S)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-1- (piperidin-4-yl)ethan-1-one | 647.30 |
| P-0139 | 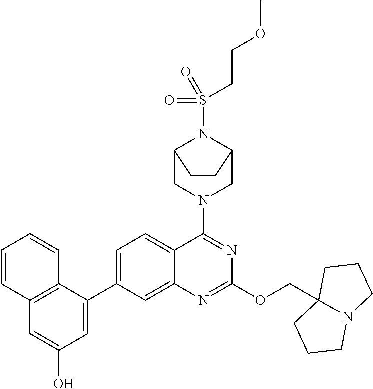 [SMILES: COCCS(=O)(=O)N1C2CCC1CN(c1nc(OCC34CCCN3CCC4)nc3cc(-c4cc(O)cc5ccccc45)ccc13)C2] | 4-(4-((1R,5S)-8-((2- methoxyethyl)sulfonyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 644.3 |
| P-0140 | 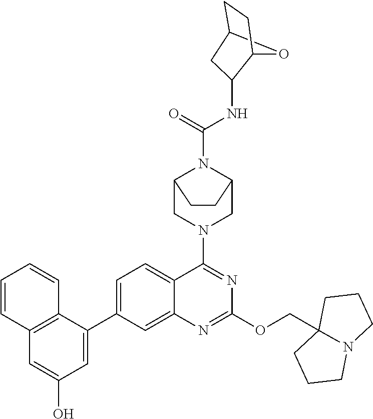 [SMILES: O=C(NC1CC2CCC1O2)N1C2CCC1CN(c1nc(OCC34CCCN3CCC4)nc3cc(-c4cc(O)cc5ccccc45)ccc13)C2] | (1R,5S)-N-((1S,2R,4R)-7- oxabicyclo[2.2.1]heptan-2-yl)-3-(7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 659.2 |
| P-0141 | 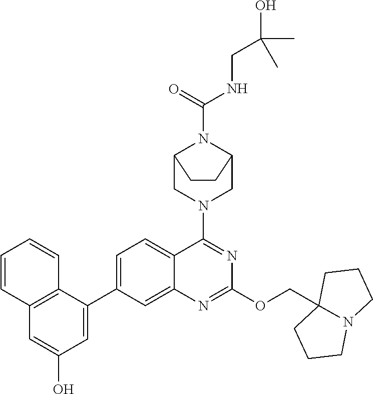 [SMILES: CC(C)(O)CNC(=O)N1C2CCC1CN(c1nc(OCC34CCCN3CCC4)nc3cc(-c4cc(O)cc5ccccc45)ccc13)C2] | (1R,5S)-N-(2-hydroxy-2- methylpropyl)-3-(7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 637.7 |
| P-0142 | 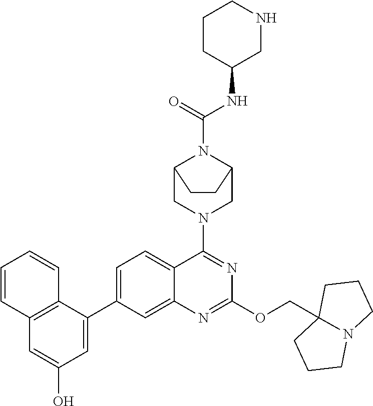 [SMILES: O=C(N[C@H]1CCCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- ((S)-piperidin-3-yl)-3,8- diazabicyclo[3.2.1]octane-8- carboxamide | 648.3 |
| P-0143 | 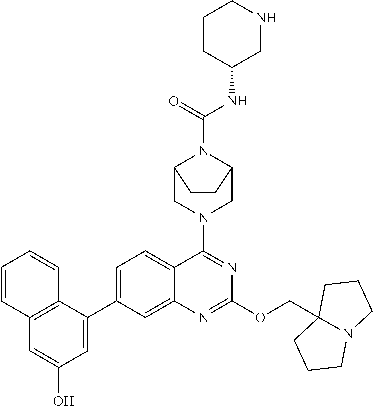 [SMILES: O=C(N[C@@H]1CCCNC1)N1C2CCC1CN(c1nc(OCC34CCCN3CCC4)nc3cc(-c4cc(O)cc5ccccc45)ccc13)C2] | (1R,5S)-3-(7-(3- hydroxynaphthalen-1-yl)-2- ((tetrahydro-1H-pyrrolizin-7a(5H)- yl)methoxy)quinazolin-4-yl)-N- ((R)-piperidin-3-yl)-3,8- diazabicyclo[3.2.1]octane-8- carboxamide | 644.3 |
| P-0144 | 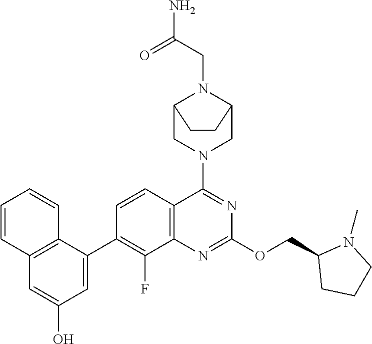 [SMILES: CN1CCC[C@H]1COc1nc(N2CC3CCC(C2)N3CC(N)=O)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8- yl)acetamide | 571.3 |
| P-0145 | 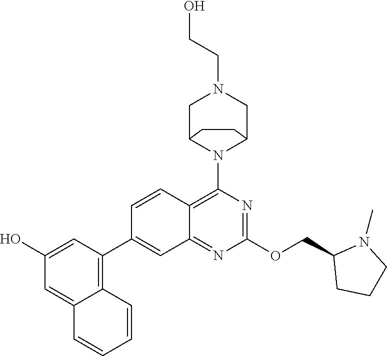 [SMILES: CN1CCC[C@H]1COc1nc(N2C3CCC2CN(CCO)C3)c2ccc(-c3cc(O)cc4ccccc34)cc2n1] | 4-(4-((1R,5S)-3-(2-hydroxyethyl)- 3,8-diazabicyclo[3.2.1]octan-8-yl)- 2-(((S)-1-methylpyrrolidin-2- yl)methoxy)quinazolin-7- yl)naphthalen-2-ol | 540.3 |
| P-0146 | 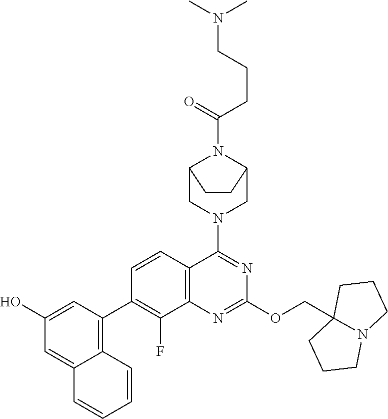 [SMILES: CN(C)CCCC(=O)N1C2CCC1CN(c1nc(OCC34CCCN3CCC4)nc3c(F)c(-c4cc(O)cc5ccccc45)ccc13)C2] | 4-(dimethylamino)-1-((1R,5S)-3-(8- fluoro-7-(3-hydroxynaphthalen-1- yl)-2-((tetrahydro-1H-pyrrolizin- 7a(5H)-yl)methoxy)quinazolin-4- yl)-3,8-diazabicyclo[3.2.1]octan-8- yl)butan-1-one | 653.7 |
| P-0147 | 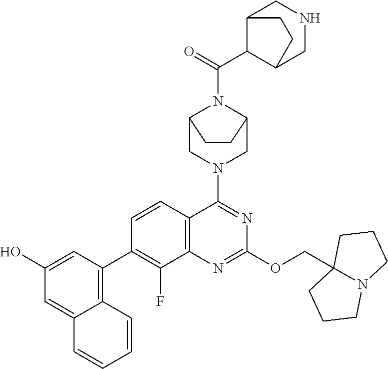 [SMILES: O=C(C1C2CCC1CNC2)N1C2CCC1CN(c1nc(OCC34CCCN3CCC4)nc3c(F)c(-c4cc(O)cc5ccccc45)ccc13)C2] | (3-azabicyclo[3.2.1]octan-8- yl)((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)methanone | 677.6 |
| P-0148 | 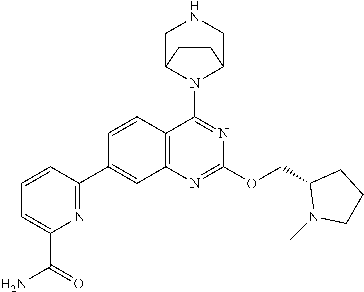 [SMILES: CN1CCC[C@H]1COc1nc(N2C3CCC2CNC3)c2ccc(-c3cccc(C(N)=O)n3)cc2n1] | 6-(4-((1R,5S)-3,8- diazabicyclo[3.2.1]octan-8-yl)-2- (((S)-1-methylpyrrolidin-2- yl)methoxy)quinazolin-7- yl)picolinamide | 474.5 |
| P-0149 | 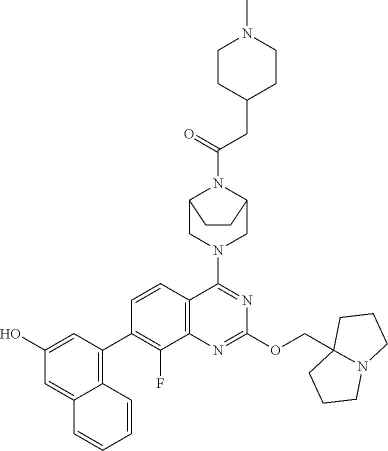 [SMILES: CN1CCC(CC(=O)N2C3CCC2CN(c2nc(OCC45CCCN4CCC5)nc4c(F)c(-c5cc(O)cc6ccccc56)ccc24)C3)CC1] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2-(1- methylpiperidin-4-yl)ethan-1-one | 679.6 |
| P-0150 | 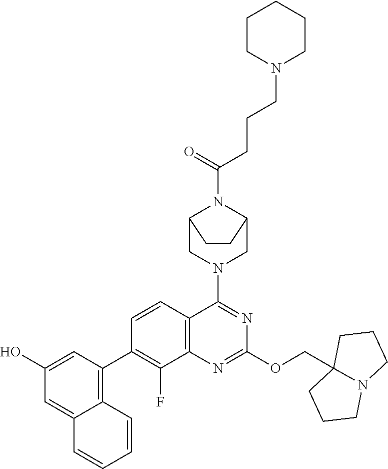 [SMILES: O=C(CCCN1CCCCC1)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-4- (piperidin-1-yl)butan-1-one | 693.7 |
| P-0151 | 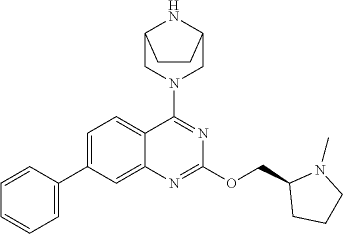 [SMILES: CN1CCC[C@H]1COc1nc(N2CC3CCC(C2)N3)c2ccc(-c3ccccc3)cc2n1] | 4-((1R,5S)-3,8- diazabicyclo[3.2.1]octan-3-yl)-2- (((S)-1-methylpyrrolidin-2- yl)methoxy)-7-phenylquinazoline | 430.3 |
| P-0152 | 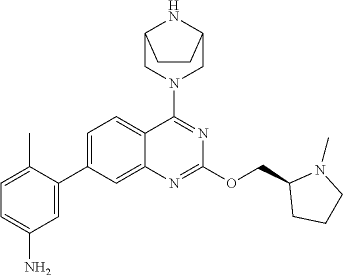 [SMILES: Cc1ccc(N)cc1-c1ccc2c(N3CC4CCC(C3)N4)nc(OC[C@@H]3CCCN3C)nc2c1] | 3-(4-((1R,5S)-3,8- diazabicyclo[3.2.1]octan-3-yl)-2- (((S)-1-methylpyrrolidin-2- yl)methoxy)quinazolin-7-yl)-4- methylaniline | 459.4 |
| P-0153 | 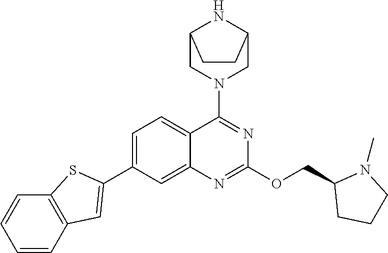 [SMILES: CN1CCC[C@H]1COc1nc(N2CC3CCC(C2)N3)c2ccc(-c3cc4ccccc4s3)cc2n1] | 7-(benzo[b]thiophen-2-yl)-4- ((1R,5S)-3,8- diazabicyclo[3.2.1]octan-3-yl)-2- (((S)-1-methylpyrrolidin-2- yl)methoxy)quinazoline | 486.4 |
| P-0154 | 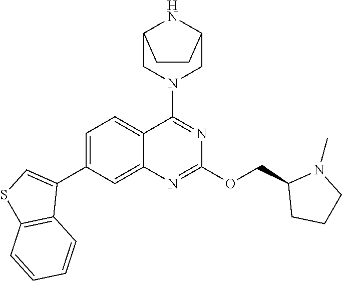 [SMILES: CN1CCC[C@H]1COc1nc(N2CC3CCC(C2)N3)c2ccc(-c3csc4ccccc34)cc2n1] | 7-(benzo[b]thiophen-3-yl)-4- ((1R,5S)-3,8- diazabicyclo[3.2.1]octan-3-yl)-2- (((S)-1-methylpyrrolidin-2- yl)methoxy)quinazoline | 486.4 |
| P-0155 | 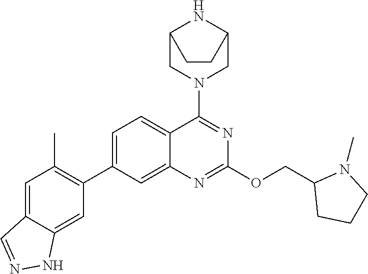 [SMILES: Cc1cc2cn[nH]c2cc1-c1ccc2c(N3CC4CCC(C3)N4)nc(OCC3CCCN3C)nc2c1] | 4-((1R,5S)-3,8- diazabicyclo[3.2.1]octan-3-yl)-7-(5- methyl-1H-indazol-6-yl)-2-((1- methylpyrrolidin-2- yl)methoxy)quinazoline | 484.3 |
| P-0156 | 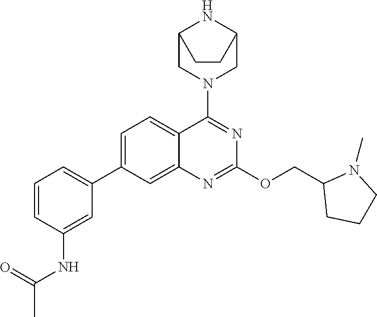 [SMILES: CC(=O)Nc1cccc(-c2ccc3c(N4CC5CCC(C4)N5)nc(OCC4CCCN4C)nc3c2)c1] | N-(3-(4-((1R,5S)-3,8- diazabicyclo[3.2.1]octan-3-yl)-2- ((1-methylpyrrolidin-2- yl)methoxy)quinazolin-7- yl)phenyl)acetamide | 487.6 |
| P-0157 | 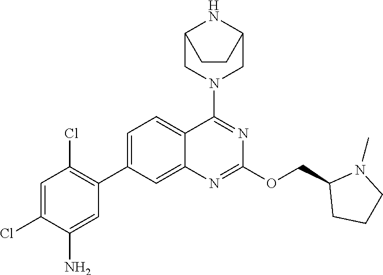 [SMILES: CN1CCC[C@H]1COc1nc(N2CC3CCC(C2)N3)c2ccc(-c3cc(N)c(Cl)cc3Cl)cc2n1] | 5-(4-((1R,5S)-3,8- diazabicyclo[3.2.1]octan-3-yl)-2- (((S)-1-methylpyrrolidin-2- yl)methoxy)quinazolin-7-yl)-2,4- dichloroaniline | 513.1 |
| P-0158 | 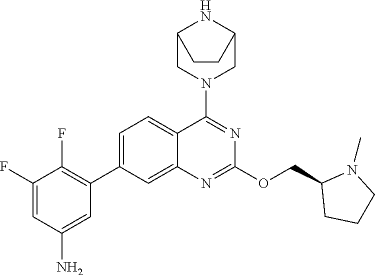 [SMILES: CN1CCC[C@H]1COc1nc(N2CC3CCC(C2)N3)c2ccc(-c3cc(N)cc(F)c3F)cc2n1] | 3-(4-((1R,5S)-3,8- diazabicyclo[3.2.1]octan-3-yl)-2- (((S)-1-methylpyrrolidin-2- yl)methoxy)quinazolin-7-yl)-4,5- difluoroaniline | 481.3 |
| P-0159 | 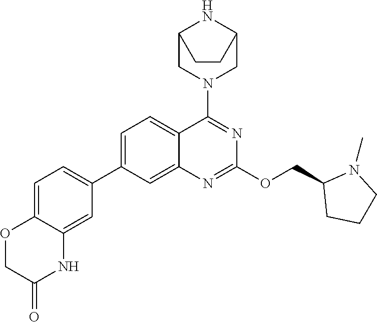 [SMILES: CN1CCC[C@H]1COc1nc(N2CC3CCC(C2)N3)c2ccc(-c3ccc4c(c3)NC(=O)CO4)cc2n1] | 6-(4-((1R,5S)-3,8- diazabicyclo[3.2.1]octan-3-yl)-2- (((S)-1-methylpyrrolidin-2- yl)methoxy)quinazolin-7-yl)-2H- benzo[b][1,4]oxazin-3(4H)-one | 501.4 |
| P-0160 | 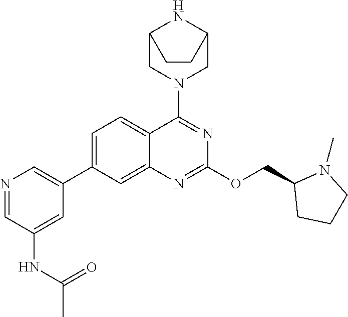 [SMILES: CC(=O)Nc1cncc(-c2ccc3c(N4CC5CCC(C4)N5)nc(OC[C@@H]4CCCN4C)nc3c2)c1] | N-(5-(4-((1R,5S)-3,8- diazabicyclo[3.2.1]octan-3-yl)-2- (((S)-1-methylpyrrolidin-2- yl)methoxy)quinazolin-7- yl)pyridin-3-yl)acetamide | 488.5 |
| P-0161 | 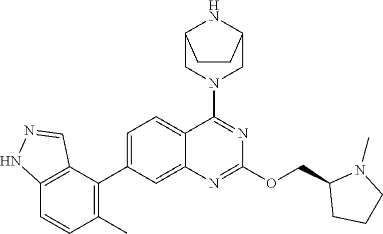 [SMILES: Cc1ccc2[nH]ncc2c1-c1ccc2c(N3CC4CCC(C3)N4)nc(OC[C@@H]3CCCN3C)nc2c1] | 4-((1R,5S)-3,8- diazabicyclo[3.2.1]octan-3-yl)-7-(5- methyl-1H-indazol-4-yl)-2-(((S)-1- methylpyrrolidin-2- yl)methoxy)quinazoline | 484.6 |
| P-0162 | 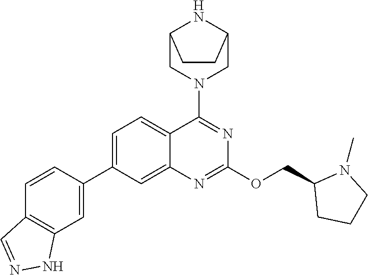 [SMILES: CN1CCC[C@H]1COc1nc(N2CC3CCC(C2)N3)c2ccc(-c3ccc4cn[nH]c4c3)cc2n1] | 4-((1R,5S)-3,8- diazabicyclo[3.2.1]octan-3-yl)-7- (1H-indazol-6-yl)-2-(((S)-1- methylpyrrolidin-2- yl)methoxy)quinazoline | 470.5 |
| P-0163 | 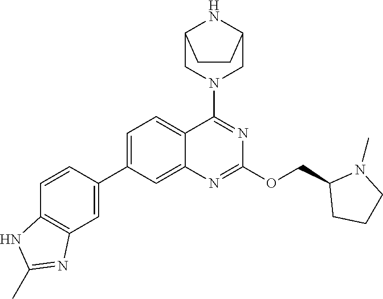 [SMILES: Cc1nc2cc(-c3ccc4c(N5CC6CCC(C5)N6)nc(OC[C@@H]5CCCN5C)nc4c3)ccc2[nH]1] | 4-((1R,5S)-3,8- diazabicyclo[3.2.1]octan-3-yl)-7-(2- methyl-1H-benzo[d]imidazol-5-yl)- 2-(((S)-1-methylpyrrolidin-2- yl)methoxy)quinazoline | 484.6 |
| P-0164 | 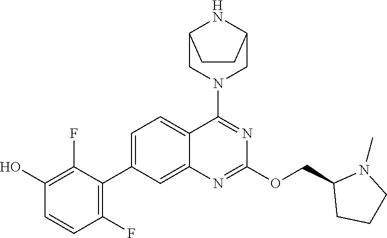 [SMILES: CN1CCC[C@H]1COc1nc(N2CC3CCC(C2)N3)c2ccc(-c3c(F)ccc(O)c3F)cc2n1] | 3-(4-((1R,5S)-3,8- diazabicyclo[3.2.1]octan-3-yl)-2- (((S)-1-methylpyrrolidin-2- yl)methoxy)quinazolin-7-yl)-2,4- difluorophenol | 482.5 |
| P-0165 | 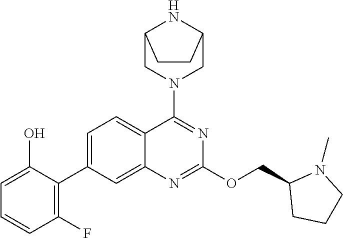 [SMILES: CN1CCC[C@H]1COc1nc(N2CC3CCC(C2)N3)c2ccc(-c3c(O)cccc3F)cc2n1] | 2-(4-((1R,5S)-3,8- diazabicyclo[3.2.1]octan-3-yl)-2- (((S)-1-methylpyrrolidin-2- yl)methoxy)quinazolin-7-yl)-3- fluorophenol | 464.2 |
| P-0166 | 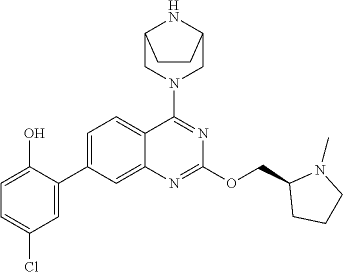 [SMILES: CN1CCC[C@H]1COc1nc(N2CC3CCC(C2)N3)c2ccc(-c3cc(Cl)ccc3O)cc2n1] | 2-(4-((1R,5S)-3,8- diazabicyclo[3.2.1]octan-3-yl)-2- (((S)-1-methylpyrrolidin-2- yl)methoxy)quinazolin-7-yl)-4- chlorophenol | 480.4 |
| P-0167 | 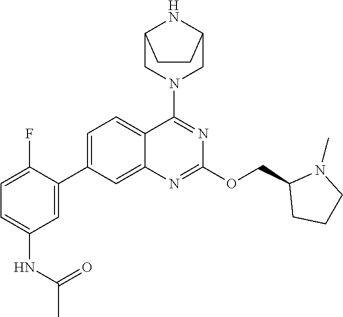 [SMILES: CC(=O)Nc1ccc(F)c(-c2ccc3c(N4CC5CCC(C4)N5)nc(OC[C@@H]4CCCN4C)nc3c2)c1] | N-(3-(4-((1R,5S)-3,8- diazabicyclo[3.2.1]octan-3-yl)-2- (((S)-1-methylpyrrolidin-2- yl)methoxy)quinazolin-7-yl)-4- fluorophenyl)acetamide | 505.3 |
| P-0168 | 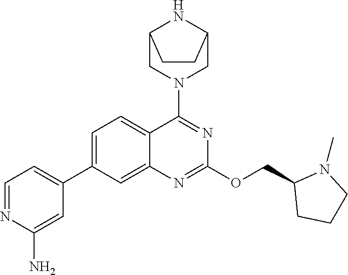 [SMILES: CN1CCC[C@H]1COc1nc(N2CC3CCC(C2)N3)c2ccc(-c3ccnc(N)c3)cc2n1] | 4-(4-((1R,5S)-3,8- diazabicyclo[3.2.1]octan-3-yl)-2- (((S)-1-methylpyrrolidin-2- yl)methoxy)quinazolin-7- yl)pyridin-2-amine | 446.5 |
| P-0169 | 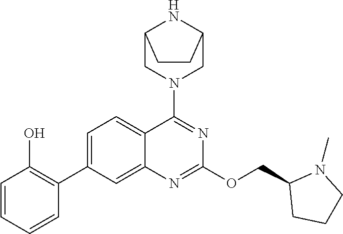 [SMILES: CN1CCC[C@H]1COc1nc(N2CC3CCC(C2)N3)c2ccc(-c3ccccc3O)cc2n1] | 2-(4-((1R,5S)-3,8- diazabicyclo[3.2.1]octan-3-yl)-2- (((S)-1-methylpyrrolidin-2- yl)methoxy)quinazolin-7-yl)phenol | 446.5 |
| P-0170 | 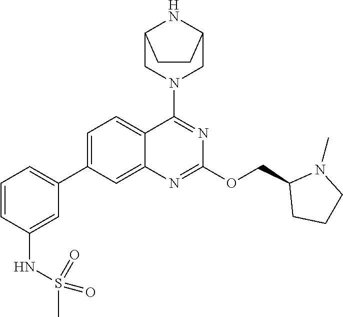 [SMILES: CN1CCC[C@H]1COc1nc(N2CC3CCC(C2)N3)c2ccc(-c3cccc(NS(C)(=O)=O)c3)cc2n1] | N-(3-(4-((1R,5S)-3,8- diazabicyclo[3.2.1]octan-3-yl)-2- (((S)-1-methylpyrrolidin-2- yl)methoxy)quinazolin-7- yl)phenyl)meanesulfonamide | 523.6 |
| P-0171 | 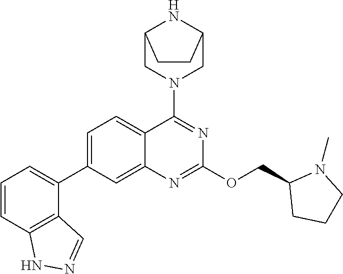 [SMILES: CN1CCC[C@H]1COc1nc(N2CC3CCC(C2)N3)c2ccc(-c3cccc4[nH]ncc34)cc2n1] | 4-((1R,5S)-3,8- diazabicyclo[3.2.1]octan-3-yl)-7- (1H-indazol-4-yl)-2-(((S)-1- methylpyrrolidin-2- yl)methoxy)quinazoline | 470.5 |
| P-0172 | 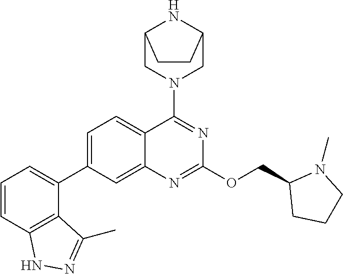 [SMILES: Cc1n[nH]c2cccc(-c3ccc4c(N5CC6CCC(C5)N6)nc(OC[C@@H]5CCCN5C)nc4c3)c12] | 4-((1R,5S)-3,8- diazabicyclo[3.2.1]octan-3-yl)-7-(3- methyl-1H-indazol-4-yl)-2-(((S)-1- methylpyrrolidin-2- yl)methoxy)quinazoline | 484.3 |
| P-0173 | 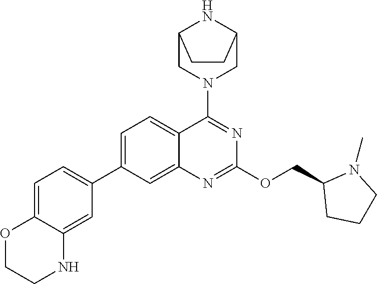 [SMILES: CN1CCC[C@H]1COc1nc(N2CC3CCC(C2)N3)c2ccc(-c3ccc4c(c3)NCCO4)cc2n1] | 6-(4-((1R,5S)-3,8- diazabicyclo[3.2.1]octan-3-yl)-2- (((S)-1-methylpyrrolidin-2- yl)methoxy)quinazolin-7-yl)-3,4- dihydro-2H-benzo[b][1,4]oxazine | 487.6 |
| P-0174 | 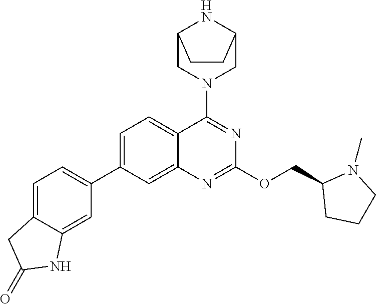 [SMILES: CN1CCC[C@H]1COc1nc(N2CC3CCC(C2)N3)c2ccc(-c3ccc4c(c3)NC(=O)C4)cc2n1] | 6-(4-((1R,5S)-3,8- diazabicyclo[3.2.1]octan-3-yl)-2- (((S)-1-methylpyrrolidin-2- yl)methoxy)quinazolin-7- yl)indolin-2-one | 485.5 |
| P-0175 | 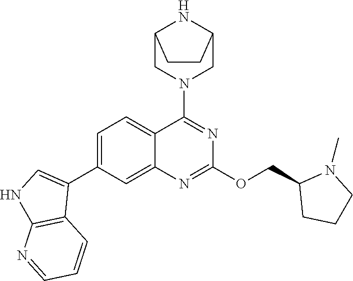 [SMILES: CN1CCC[C@H]1COc1nc(N2CC3CCC(C2)N3)c2ccc(-c3c[nH]c4ncccc34)cc2n1] | 4-((1R,5S)-3,8- diazabicyclo[3.2.1]octan-3-yl)-2- (((S)-1-methylpyrrolidin-2- yl)methoxy)-7-(1H-pyrrolo[2,3- b]pyridin-3-yl)quinazoline | 470.2 |
| P-0176 | 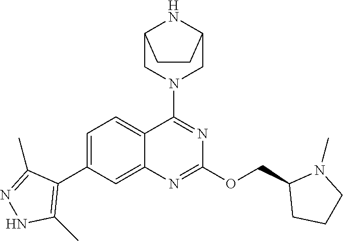 [SMILES: Cc1n[nH]c(C)c1-c1ccc2c(N3CC4CCC(C3)N4)nc(OC[C@@H]3CCCN3C)nc2c1] | 4-((1R,5S)-3,8- diazabicyclo[3.2.1]octan-3-yl)-7- (3,5-dimethyl-1H-pyrazol-4-yl)-2- (((S)-1-methylpyrrolidin-2- yl)methoxy)quinazoline | 448.3 |
| P-0177 | 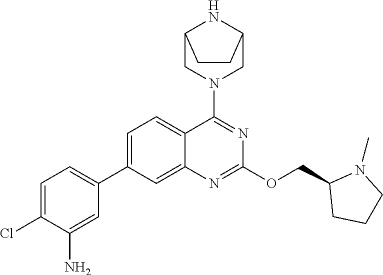 [SMILES: CN1CCC[C@H]1COc1nc(N2CC3CCC(C2)N3)c2ccc(-c3ccc(Cl)c(N)c3)cc2n1] | 5-(4-((1R,5S)-3,8- diazabicyclo[3.2.1]octan-3-yl)-2- (((S)-1-methylpyrrolidin-2- yl)methoxy)quinazolin-7-yl)-2- chloroaniline | 479.5 |
| P-0178 | 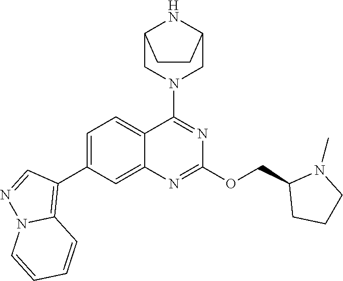 [SMILES: CN1CCC[C@H]1COc1nc(N2CC3CCC(C2)N3)c2ccc(-c3cnn4ccccc34)cc2n1] | 4-((1R,5S)-3,8- diazabicyclo[3.2.1]octan-3-yl)-2- (((S)-1-methylpyrrolidin-2- yl)methoxy)-7-(pyrazolo[1,5- a]pyridin-3-yl)quinazoline | 470.8 |
| P-0179 | 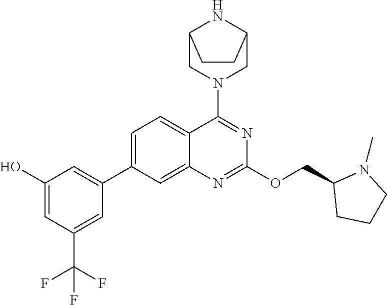 [SMILES: CN1CCC[C@H]1COc1nc(N2CC3CCC(C2)N3)c2ccc(-c3cc(O)cc(C(F)(F)F)c3)cc2n1] | 3-(4-((1R,5S)-3,8- diazabicyclo[3.2.1]octan-3-yl)-2- (((S)-1-methylpyrrolidin-2- yl)methoxy)quinazolin-7-yl)-5- (trifluoromethyl)phenol | 514.3 |
| P-0180 | 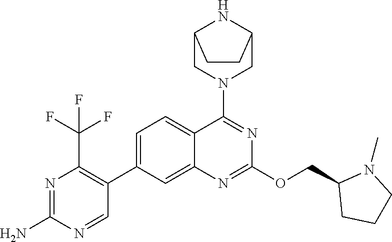 [SMILES: CN1CCC[C@H]1COc1nc(N2CC3CCC(C2)N3)c2ccc(-c3cnc(N)nc3C(F)(F)F)cc2n1] | 5-(4-((1R,5S)-3,8- diazabicyclo[3.2.1]octan-3-yl)-2- (((S)-1-methylpyrrolidin-2- yl)methoxy)quinazolin-7-yl)-4- (trifluoromethyl)pyrimidin-2-amine | 515.5 |
| P-0181 | 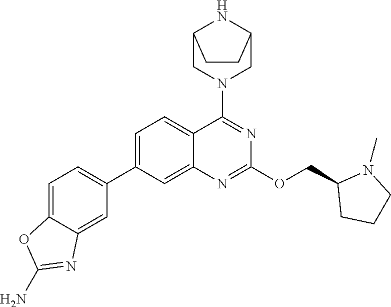 [SMILES: CN1CCC[C@H]1COc1nc(N2CC3CCC(C2)N3)c2ccc(-c3ccc4oc(N)nc4c3)cc2n1] | 5-(4-((1R,5S)-3,8- diazabicyclo[3.2.1]octan-3-yl)-2- (((S)-1-methylpyrrolidin-2- yl)methoxy)quinazolin-7- yl)benzo[d]oxazol-2-amine | 486.4 |
| P-0182 | 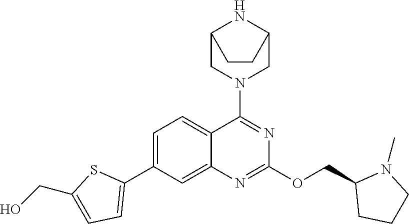 [SMILES: CN1CCC[C@H]1COc1nc(N2CC3CCC(C2)N3)c2ccc(-c3ccc(CO)s3)cc2n1] | (5-(4-((1R,5S)-3,8- diazabicyclo[3.2.1]octan-3-yl)-2- (((S)-1-methylpyrrolidin-2- yl)methoxy)quinazolin-7- yl)thiophen-2-yl)methanol | 466.3 |
| P-0183 | 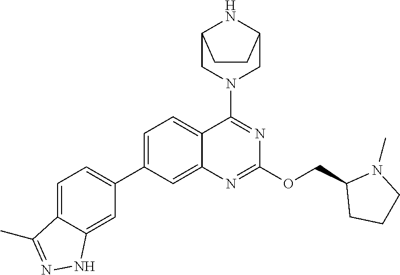 [SMILES: Cc1n[nH]c2cc(-c3ccc4c(N5CC6CCC(C5)N6)nc(OC[C@@H]5CCCN5C)nc4c3)ccc12] | 4-((1R,5S)-3,8- diazabicyclo[3.2.1]octan-3-yl)-7-(3- methyl-1H-indazol-6-yl)-2-(((S)-1- methylpyrrolidin-2- yl)methoxy)quinazoline | 484.3 |
| P-0184 | 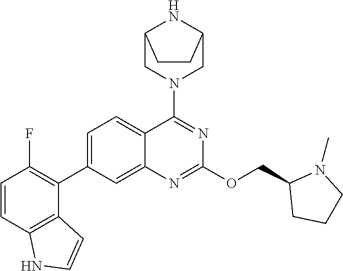 [SMILES: CN1CCC[C@H]1COc1nc(N2CC3CCC(C2)N3)c2ccc(-c3c(F)ccc4[nH]ccc34)cc2n1] | 4-((1R,5S)-3,8- diazabicyclo[3.2.1]octan-3-yl)-7-(5- fluoro-1H-indol-4-yl)-2-(((S)-1- methylpyrrolidin-2- yl)methoxy)quinazoline | 487.6 |
| P-0185 | 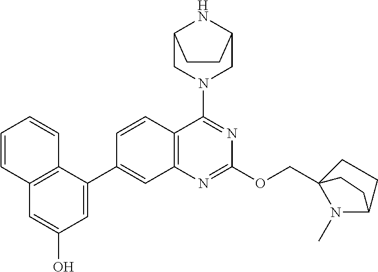 [SMILES: CN1C2CCC1(COc1nc(N3CC4CCC(C3)N4)c3ccc(-c4cc(O)cc5ccccc45)cc3n1)CC2] | 4-(4-((1R,5S)-3,8- diazabicyclo[3.2.1]octan-3-yl)-2- ((7-methyl-7- azabicyclo[2.2.1]heptan-1- yl)methoxy)quinazolin-7- yl)naphthalen-2-ol | 522.3 |
| P-0186 | 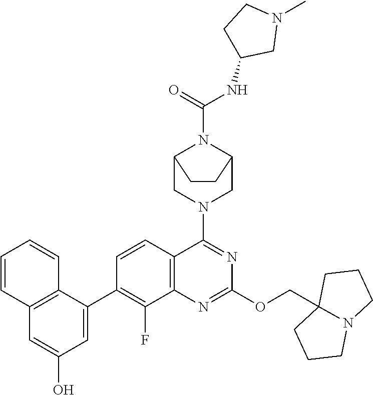 [SMILES: CN1CC[C@@H](NC(=O)N2C3CCC2CN(c2nc(OCC45CCCN4CCC5)nc4c(F)c(-c5cc(O)cc6ccccc56)ccc24)C3)C1] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-N- ((R)-1-methylpyrrolidin-3-yl)-3,8- diazabicyclo[3.2.1]octane-8- carboxamide | 666.3 |
| P-0187 | 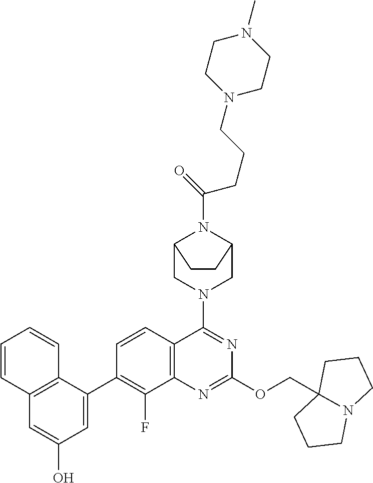 [SMILES: CN1CCN(CCCC(=O)N2C3CCC2CN(c2nc(OCC45CCCN4CCC5)nc4c(F)c(-c5cc(O)cc6ccccc56)ccc24)C3)CC1] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-4-(4- methylpiperazin-1-yl)butan-1-one | 708.3 |
| P-0188 | 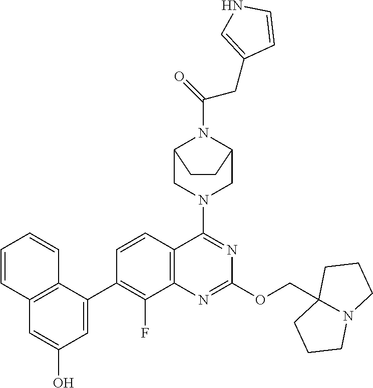 [SMILES: O=C(Cc1cc[nH]c1)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- (1H-imidazol-4-yl)ethan-1-one | 648.3 |
| P-0189 | 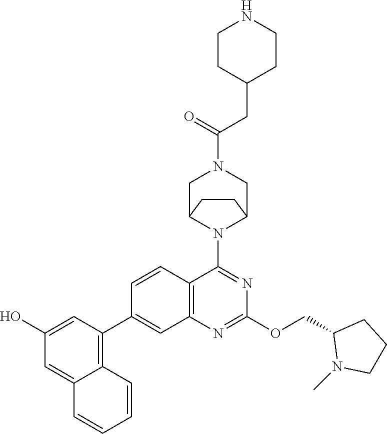 [SMILES: CN1CCC[C@H]1COc1nc(N2C3CCC2CN(C(=O)CC2CCNCC2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- (piperidin-4-yl)ethan-1-one | 621.4 |
| P-0190 | 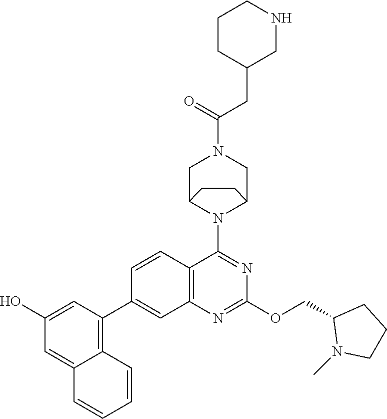 [SMILES: CN1CCC[C@H]1COc1nc(N2C3CCC2CN(C(=O)CC2CCCNC2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- (piperidin-3-yl)ethan-1-one | 621.5 |
| P-0191 | 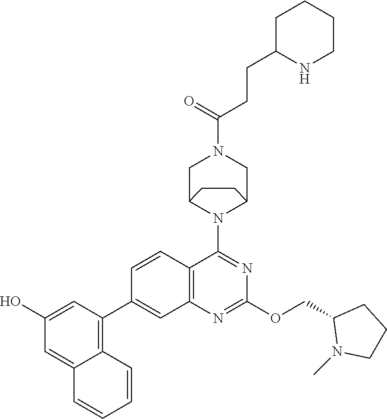 [SMILES: CN1CCC[C@H]1COc1nc(N2C3CCC2CN(C(=O)CCC2CCCCN2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- (piperidin-2-yl)propan-1-one | 635.5 |
| P-0192 | 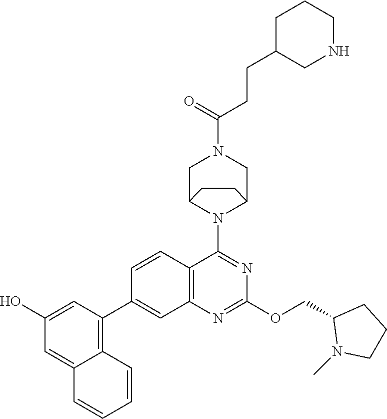 [SMILES: CN1CCC[C@H]1COc1nc(N2C3CCC2CN(C(=O)CCC2CCCNC2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- (piperidin-3-yl)propan-1-one | 635.5 |
| P-0193 | 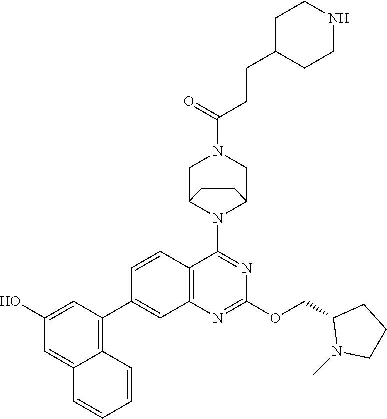 [SMILES: CN1CCC[C@H]1COc1nc(N2C3CCC2CN(C(=O)CCC2CCNCC2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- (piperidin-4-yl)propan-1-one | 635.6 |
| P-0194 | 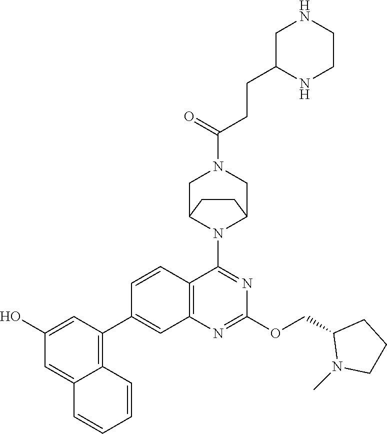 [SMILES: CN1CCC[C@H]1COc1nc(N2C3CCC2CN(C(=O)CCC2CNCCN2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- (piperazin-2-yl)propan-1-one | 636.6 |
| P-0195 | 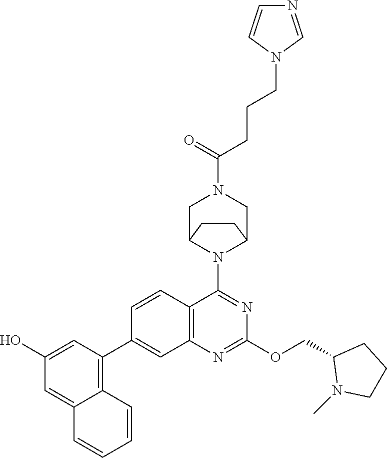 [SMILES: CN1CCC[C@H]1COc1nc(N2C3CCC2CN(C(=O)CCCn2ccnc2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-4- (1H-imidazol-1-yl)butan-1-one | 632.5 |
| P-0196 | 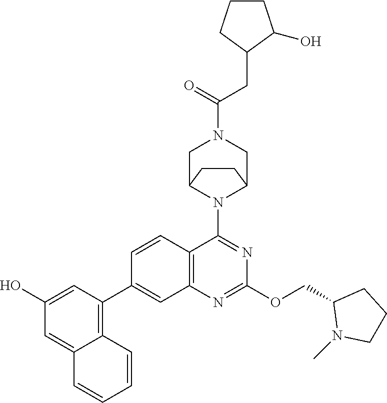 [SMILES: CN1CCC[C@H]1COc1nc(N2C3CCC2CN(C(=O)CC2CCCC2O)C3)c2ccc(-c3cc(O)cc4ccccc34)cc2n1] | 2-(2-hydroxycyclopentyl)-1- ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)ethan-1-one | 622.5 |
| P-0197 | 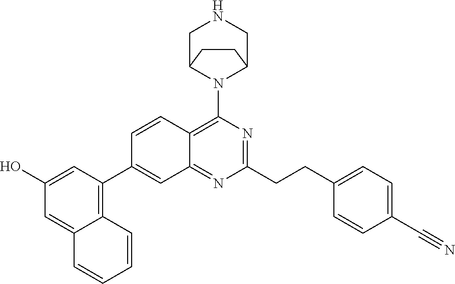 [SMILES: N#Cc1ccc(CCc2nc(N3C4CCC3CNC4)c3ccc(-c4cc(O)cc5ccccc45)cc3n2)cc1] | 4-(2-(4-((1R,5S)-3,8- diazabicyclo[3.2.1]octan-8-yl)-7-(3- hydroxynaphthalen-1-yl)quinazolin- 2-yl)ethyl)benzonitrile | 512.2 |
| P-0198 | 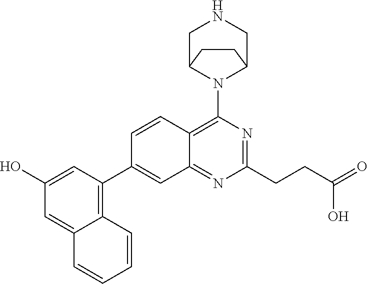 [SMILES: O=C(O)CCc1nc(N2C3CCC2CNC3)c2ccc(-c3cc(O)cc4ccccc34)cc2n1] | 3-(4-((1R,5S)-3,8- diazabicyclo[3.2.1]octan-8-yl)-7-(3- hydroxynaphthalen-1-yl)quinazolin- 2-yl)propanoic acid | 455.1 |
| P-0199 | 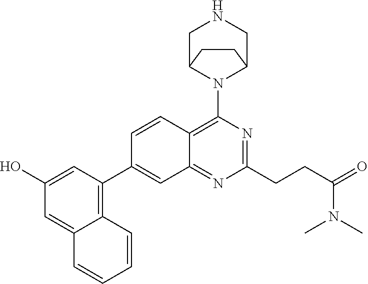 [SMILES: CN(C)C(=O)CCc1nc(N2C3CCC2CNC3)c2ccc(-c3cc(O)cc4ccccc34)cc2n1] | 3-(4-((1R,5S)-3,8- diazabicyclo[3.2.1]octan-8-yl)-7-(3- hydroxynaphthalen-1-yl)quinazolin- 2-yl)-N,N-dimethylpropanamide | 482.2 |
| P-0200 | 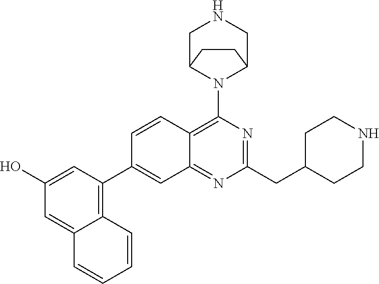 [SMILES: Oc1cc(-c2ccc3c(N4C5CCC4CNC5)nc(CC4CCNCC4)nc3c2)c2ccccc2c1] | 4-(4-((1R,5S)-3,8- diazabicyclo[3.2.1]octan-8-yl)-2- (piperidin-4-ylmethyl)quinazolin-7- yl)naphthalen-2-ol | 480.3 |
| P-0201 | 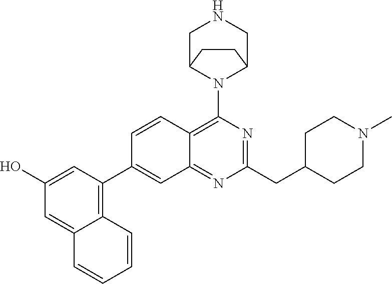 [SMILES: CN1CCC(Cc2nc(N3C4CCC3CNC4)c3ccc(-c4cc(O)cc5ccccc45)cc3n2)CC1] | 4-(4-((1R,5S)-3,8- diazabicyclo[3.2.1]octan-8-yl)-2- ((1-methylpiperidin-4- yl)methyl)quinazolin-7- yl)naphthalen-2-ol | 494.2 |
| P-0202 | 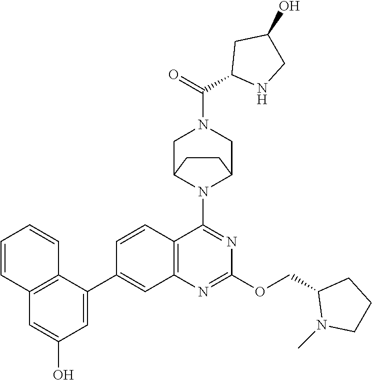 [SMILES: CN1CCC[C@H]1COc1nc(N2C3CCC2CN(C(=O)[C@@H]2C[C@@H](O)CN2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)((2S,4R)-4-hydroxypyrrolidin-2- yl)methanone | 496.6 |
| P-0203 | 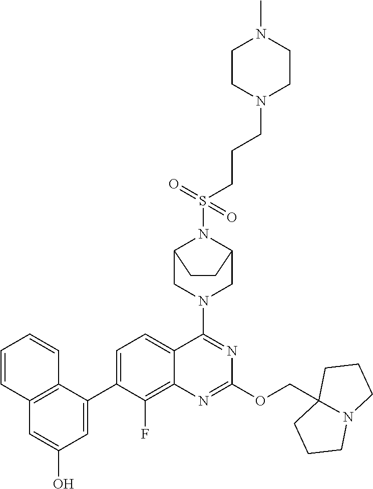 [SMILES: CN1CCN(CCCS(=O)(=O)N2C3CCC2CN(c2nc(OCC45CCCN4CCC5)nc4c(F)c(-c5cc(O)cc6ccccc56)ccc24)C3)CC1] | 4-(8-fluoro-4-((1R,5S)-8-((3-(4- methylpiperazin-1- yl)propyl)sulfonyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 744.3 |
| P-0204 | 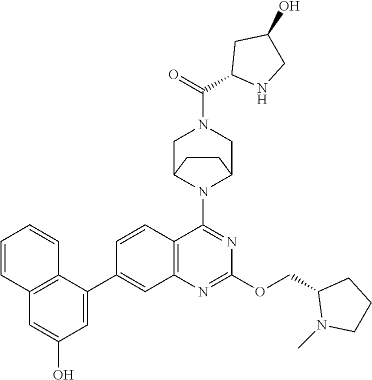 [SMILES: CN1CCC[C@H]1COc1nc(N2C3CCC2CN(C(=O)[C@@H]2C[C@@H](O)CN2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)((2S,4R)-4-hydroxypyrrolidin-2- yl)methanone | 609.4 |
| P-0205 | 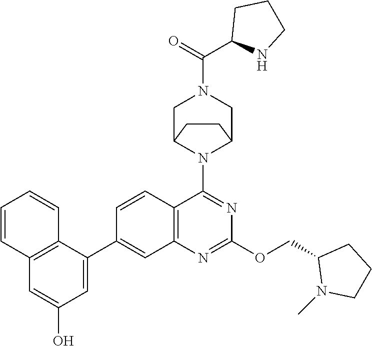 [SMILES: CN1CCC[C@H]1COc1nc(N2C3CCC2CN(C(=O)[C@H]2CCCN2)C3)c2ccc(-c3cc(O)cc4ccccc34)cc2n1] | 4-((1R,5S)-3-(D-prolyl)-3,8- diazabicyclo[3.2.1]octan-8-yl)-7-(3- hydroxynaphthalen-1-yl)-2-(((S)-1- methylpyrrolidin-2- yl)methoxy)quinazoline | 593.5 |
| P-0206 | 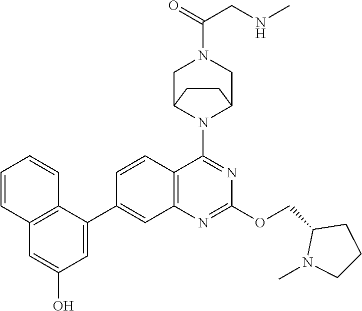 [SMILES: CNCC(=O)N1CC2CCC(C1)N2c1nc(OC[C@@H]2CCCN2C)nc2cc(-c3cc(O)cc4ccccc34)ccc12] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- (methylamino)ethan-1-one | 567.4 |
| P-0207 | 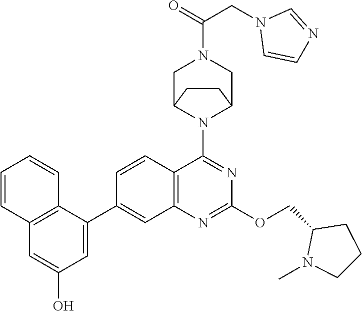 [SMILES: CN1CCC[C@H]1COc1nc(N2C3CCC2CN(C(=O)Cn2ccnc2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- (1H-imidazol-1-yl)ethan-1-one | 604.6 |
| P-0208 | 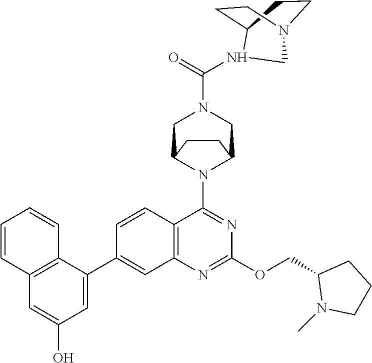 | (1R,5S)-8-(7-(3- hydroxynaphthalen-1-yl)-2-(((S)-1- methylpyrrolidin-2- yl)methoxy)quinazolin-4-yl)-N- (quinuclidin-3-yl)-3,8- diazabicyclo[3.2.1]octane-3- carboxamide | 648.7 |
| P-0209 | 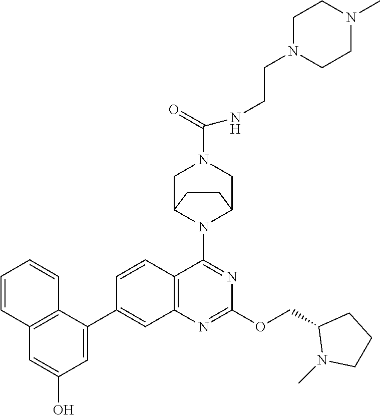 [SMILES: CN1CCN(CCNC(=O)N2CC3CCC(C2)N3c2nc(OC[C@@H]3CCCN3C)nc3cc(-c4cc(O)cc5ccccc45)ccc23)CC1] | (1R,5S)-8-(7-(3- hydroxynaphthalen-1-yl)-2-(((S)-1- methylpyrrolidin-2- yl)methoxy)quinazolin-4-yl)-N-(2- (4-methylpiperazin-1-yl)ethyl)-3,8- diazabicyclo[3.2.1]octane-3- carboxamide | 665.5 |
| P-0210 | 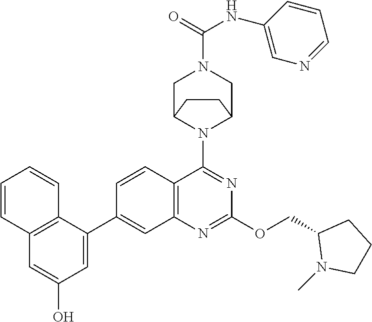 [SMILES: CN1CCC[C@H]1COc1nc(N2C3CCC2CN(C(=O)Nc2cccnc2)C3)c2ccc(-c3cc(O)cc4ccccc34)cc2n1] | (1R,5S)-8-(7-(3- hydroxynaphthalen-1-yl)-2-(((S)-1- methylpyrrolidin-2- yl)methoxy)quinazolin-4-yl)-N- (pyridin-3-yl)-3,8- diazabicyclo[3.2.1]octane-3- carboxamide | 616.6 |
| P-0211 | 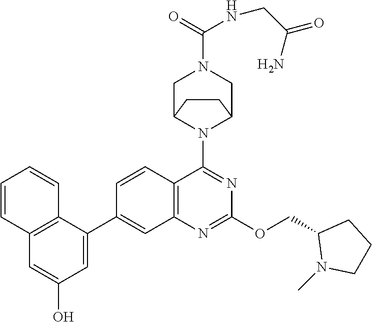 [SMILES: CN1CCC[C@H]1COc1nc(N2C3CCC2CN(C(=O)NCC(N)=O)C3)c2ccc(-c3cc(O)cc4ccccc34)cc2n1] | (1R,5S)-N-(2-amino-2-oxoethyl)-8- (7-(3-hydroxynaphthalen-1-yl)-2- (((S)-1-methylpyrrolidin-2- yl)methoxy)quinazolin-4-yl)-3,8- diazabicyclo[3.2.1]octane-3- carboxamide | 596.5 |
| P-0212 | 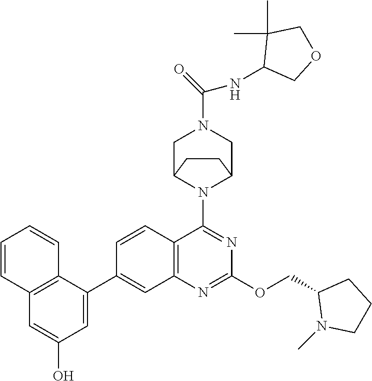 [SMILES: CN1CCC[C@H]1COc1nc(N2C3CCC2CN(C(=O)NC2COCC2(C)C)C3)c2ccc(-c3cc(O)cc4ccccc34)cc2n1] | (1R,5S)-N-(4,4- dimethyltetrahydrofuran-3-yl)-8-(7- (3-hydroxynaphthalen-1-yl)-2-(((S)- 1-methylpyrrolidin-2- yl)methoxy)quinazolin-4-yl)-3,8- diazabicyclo[3.2.1]octane-3- carboxamide | 637.3 |
| P-0213 | 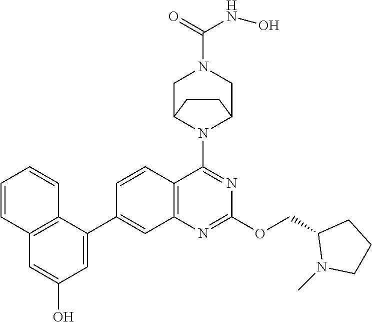 [SMILES: CN1CCC[C@H]1COc1nc(N2C3CCC2CN(C(=O)NO)C3)c2ccc(-c3cc(O)cc4ccccc34)cc2n1] | (1R,5S)-N-hydroxy-8-(7-(3- hydroxynaphthalen-1-yl)-2-(((S)-1- methylpyrrolidin-2- yl)methoxy)quinazolin-4-yl)-3,8- diazabicyclo[3.2.1]octane-3- carboxamide | 555.1 |
| P-0214 | 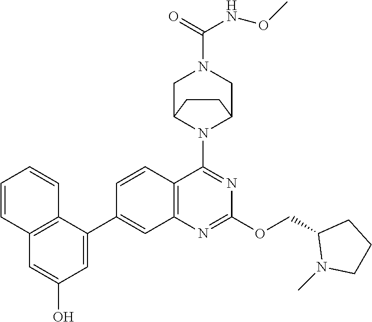 [SMILES: CONC(=O)N1CC2CCC(C1)N2c1nc(OC[C@@H]2CCCN2C)nc2cc(-c3cc(O)cc4ccccc34)ccc12] | (1R,5S)-8-(7-(3- hydroxynaphthalen-1-yl)-2-(((S)-1- methylpyrrolidin-2- yl)methoxy)quinazolin-4-yl)-N- methoxy-3,8- diazabicyclo[3.2.1]octane-3- carboxamide | 569.5 |
| P-0215 | 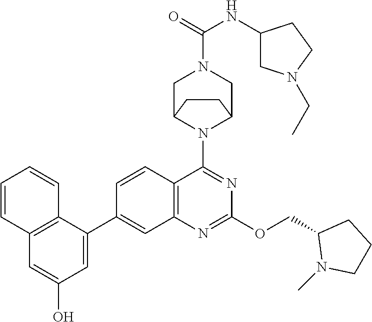 [SMILES: CCN1CCC(NC(=O)N2CC3CCC(C2)N3c2nc(OC[C@@H]3CCCN3C)nc3cc(-c4cc(O)cc5ccccc45)ccc23)C1] | (1R,5S)-N-(1-ethylpyrrolidin-3-yl)- 8-(7-(3-hydroxynaphthalen-1-yl)-2- (((S)-1-methylpyrrolidin-2- yl)methoxy)quinazolin-4-yl)-3,8- diazabicyclo[3.2.1]octane-3- carboxamide | 636.4 |
| P-0216 | 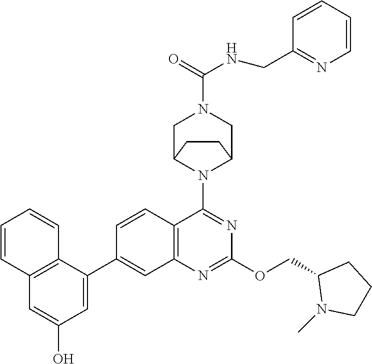 [SMILES: CN1CCC[C@H]1COc1nc(N2C3CCC2CN(C(=O)NCc2ccccn2)C3)c2ccc(-c3cc(O)cc4ccccc34)cc2n1] | (1R,5S)-8-(7-(3- hydroxynaphthalen-1-yl)-2-(((S)-1- methylpyrrolidin-2- yl)methoxy)quinazolin-4-yl)-N- (pyridin-2-ylmethyl)-3,8- diazabicyclo[3.2.1]octane-3- carboxamide | 630.7 |
| P-0217 | 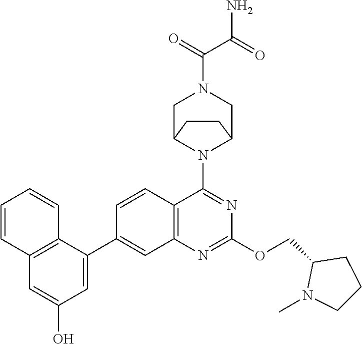 [SMILES: CN1CCC[C@H]1COc1nc(N2C3CCC2CN(C(=O)C(N)=O)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxoacetamide | 567.4 |
| P-0218 | 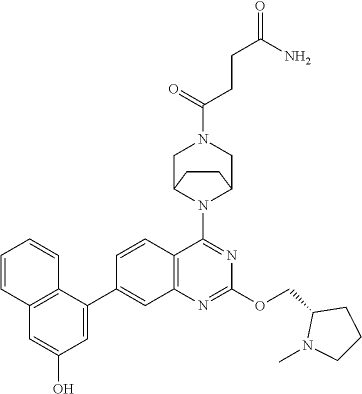 [SMILES: CN1CCC[C@H]1COc1nc(N2C3CCC2CN(C(=O)CCC(N)=O)C3)c2ccc(-c3cc(O)cc4ccccc34)cc2n1] | 4-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-4- oxobutanamide | 595.6 |
| P-0219 | 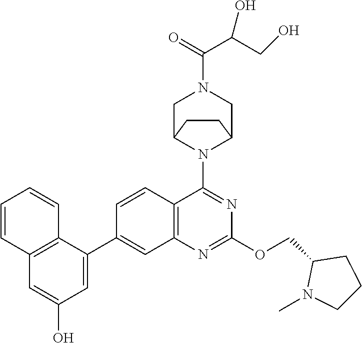 [SMILES: CN1CCC[C@H]1COc1nc(N2C3CCC2CN(C(=O)C(O)CO)C3)c2ccc(-c3cc(O)cc4ccccc34)cc2n1] | 2,3-dihydroxy-1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)propan-1-one | 584.5 |
| P-0220 | 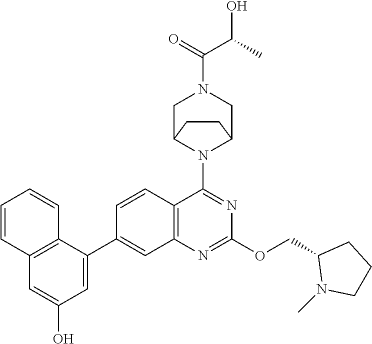 [SMILES: C[C@@H](O)C(=O)N1CC2CCC(C1)N2c1nc(OC[C@@H]2CCCN2C)nc2cc(-c3cc(O)cc4ccccc34)ccc12] | (R)-2-hydroxy-1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)propan-1-one | 568.6 |
| P-0221 | 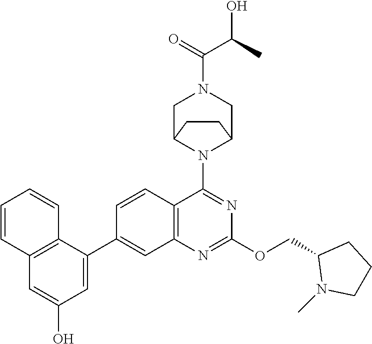 [SMILES: C[C@H](O)C(=O)N1CC2CCC(C1)N2c1nc(OC[C@@H]2CCCN2C)nc2cc(-c3cc(O)cc4ccccc34)ccc12] | (S)-2-hydroxy-1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)propan-1-one | 568.6 |
| P-0222 | 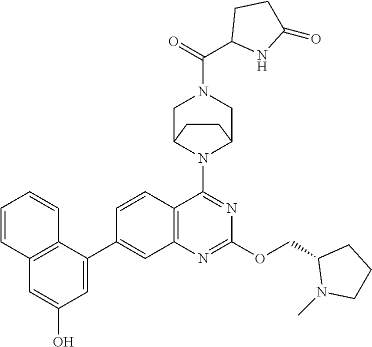 [SMILES: CN1CCC[C@H]1COc1nc(N2C3CCC2CN(C(=O)C2CCC(=O)N2)C3)c2ccc(-c3cc(O)cc4ccccc34)cc2n1] | 5-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octane-3- carbonyl)pyrrolidin-2-one | 607.6 |
| P-0223 | 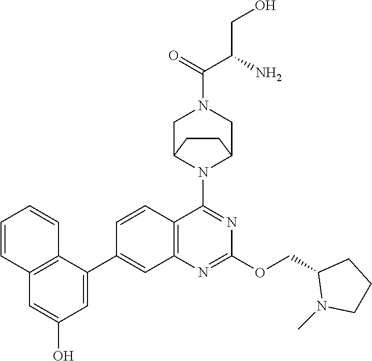 [SMILES: CN1CCC[C@H]1COc1nc(N2C3CCC2CN(C(=O)[C@@H](N)CO)C3)c2ccc(-c3cc(O)cc4ccccc34)cc2n1] | (S)-2-amino-3-hydroxy-1-((1R,5S)- 8-(7-(3-hydroxynaphthalen-1-yl)-2- (((S)-1-methylpyrrolidin-2- yl)methoxy)quinazolin-4-yl)-3,8- diazabicyclo[3.2.1]octan-3- yl)propan-1-one | 583.3 |
| P-0224 | 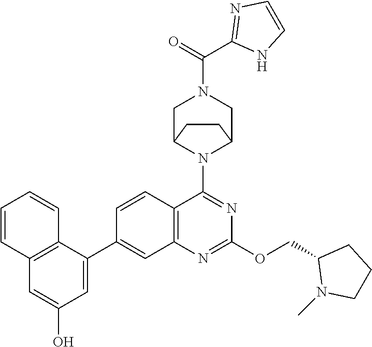 [SMILES: CN1CCC[C@H]1COc1nc(N2C3CCC2CN(C(=O)c2ncc[nH]2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)(1H- imidazol-2-yl)methanone | 590.5 |
| P-0225 | 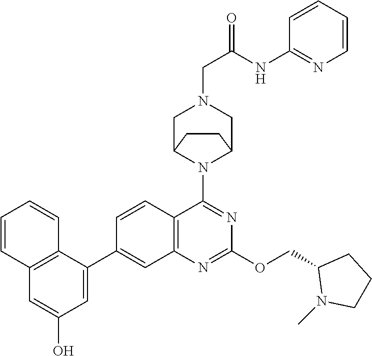 [SMILES: CN1CCC[C@H]1COc1nc(N2C3CCC2CN(CC(=O)Nc2ccccn2)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-N- (pyridin-2-yl)acetamide | 630.7 |
| P-0226 | 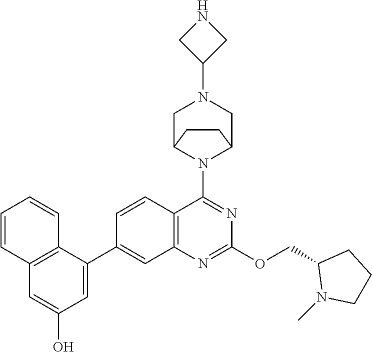 [SMILES: CN1CCC[C@H]1COc1nc(N2C3CCC2CN(C2CNC2)C3)c2ccc(-c3cc(O)cc4ccccc34)cc2n1] | 4-(4-((1R,5S)-3-(azetidin-3-yl)-3,8- diazabicyclo[3.2.1]octan-8-yl)-2- (((S)-1-methylpyrrolidin-2- yl)methoxy)quinazolin-7- yl)naphthalen-2-ol | 551.5 |
| P-0227 | 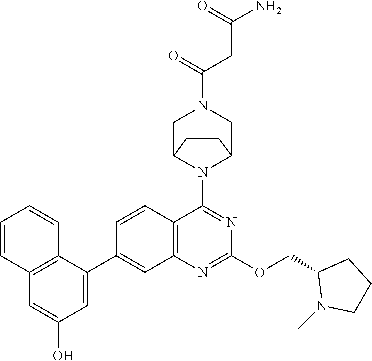 [SMILES: CN1CCC[C@H]1COc1nc(N2C3CCC2CN(C(=O)CC(N)=O)C3)c2ccc(-c3cc(O)cc4ccccc34)cc2n1] | 3-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- oxopropanamide | 581.5 |
| P-0228 | 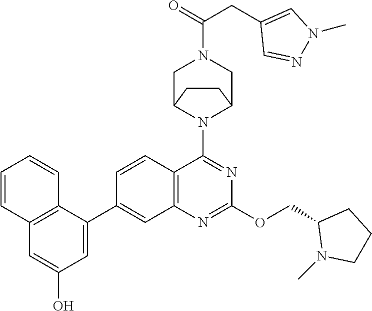 [SMILES: CN1CCC[C@H]1COc1nc(N2C3CCC2CN(C(=O)Cc2cnn(C)c2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2-(1- methyl-1H-pyrazol-4-yl)ethan-1- one | 618.7 |
| P-0229 | 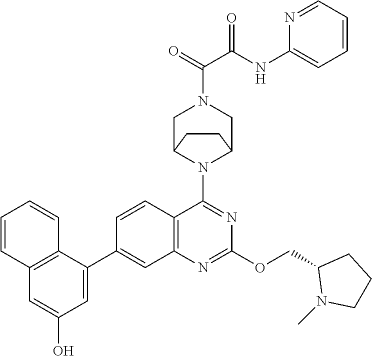 [SMILES: CN1CCC[C@H]1COc1nc(N2C3CCC2CN(C(=O)C(=O)Nc2ccccn2)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxo-N-(pyridin-2-yl)acetamide | 644.5 |
| P-0230 | 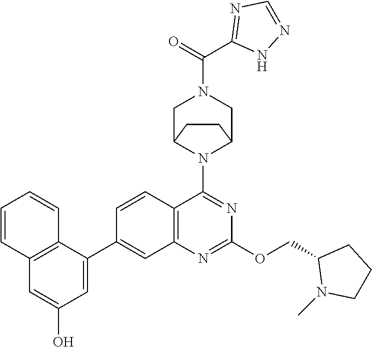 [SMILES: CN1CCC[C@H]1COc1nc(N2C3CCC2CN(C(=O)c2ncn[nH]2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)(1H- 1,2,4-triazol-5-yl)methanone | 591.4 |
| P-0231 | 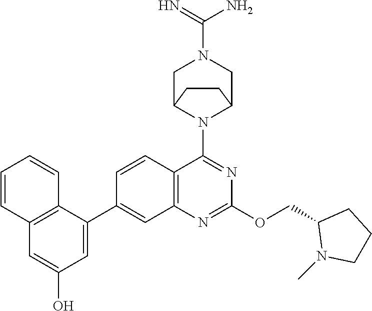 [SMILES: CN1CCC[C@H]1COc1nc(N2C3CCC2CN(C(=N)N)C3)c2ccc(-c3cc(O)cc4ccccc34)cc2n1] | (1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octane-3- carboximidamide | 638.6 |
| P-0232 | 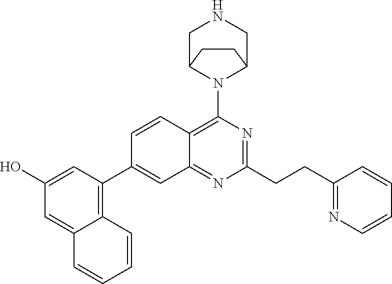 [SMILES: Oc1cc(-c2ccc3c(N4C5CCC4CNC5)nc(CCc4ccccn4)nc3c2)c2ccccc2c1] | 4-(4-((1R,5S)-3,8- diazabicyclo[3.2.1]octan-8-yl)-2-(2- (pyridin-2-yl)ethyl)quinazolin-7- yl)naphthalen-2-ol | 488.2 |
| P-0233 | 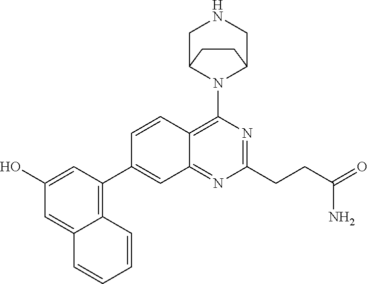 [SMILES: NC(=O)CCc1nc(N2C3CCC2CNC3)c2ccc(-c3cc(O)cc4ccccc34)cc2n1] | 3-(4-((1R,5S)-3,8- diazabicyclo[3.2.1]octan-8-yl)-7-(3- hydroxynaphthalen-1-yl)quinazolin- 2-yl)propanamide | 454.1 |
| P-0234 | 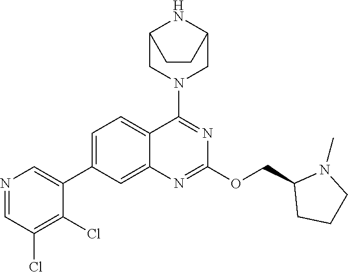 [SMILES: CN1CCC[C@H]1COc1nc(N2CC3CCC(C2)N3)c2ccc(-c3cncc(Cl)c3Cl)cc2n1] | 4-((1R,5S)-3,8- diazabicyclo[3.2.1]octan-3-yl)-7- (4,5-dichloropyridin-3-yl)-2-(((S)- 1-methylpyrrolidin-2- yl)methoxy)quinazoline | 499.1 |
| P-0235 | 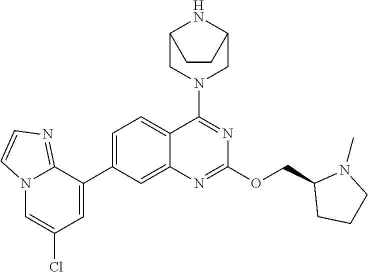 [SMILES: CN1CCC[C@H]1COc1nc(N2CC3CCC(C2)N3)c2ccc(-c3cc(Cl)cn4ccnc34)cc2n1] | 4-((1R,5S)-3,8- diazabicyclo[3.2.1]octan-3-yl)-7-(6- chloroimidazo[1,2-alpyridin-8-yl)- 2-(((S)-1-methylpyrrolidin-2- yl)methoxy)quinazoline | 504.2 |
| P-0236 | 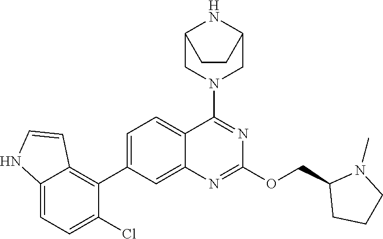 [SMILES: CN1CCC[C@H]1COc1nc(N2CC3CCC(C2)N3)c2ccc(-c3c(Cl)ccc4[nH]ccc34)cc2n1] | 4-((1R,5S)-3,8- diazabicyclo[3.2.1]octan-3-yl)-7-(5- chloro-1H-indol-4-yl)-2-(((S)-1- methylpyrrolidin-2- yl)methoxy)quinazoline | 504.2 |
| P-0237 | 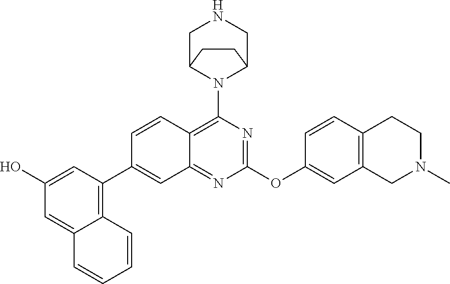 [SMILES: CN1CCc2ccc(Oc3nc(N4C5CCC4CNC5)c4ccc(-c5cc(O)cc6ccccc56)cc4n3)cc2C1] | 4-(4-((1R,5S)-3,8- diazabicyclo[3.2.1]octan-8-yl)-2- ((2-methyl-1,2,3,4- tetrahydroisoquinolin-7- yl)oxy)quinazolin-7-yl)naphthalen- 2-ol | 544.7 |
| P-0238 | 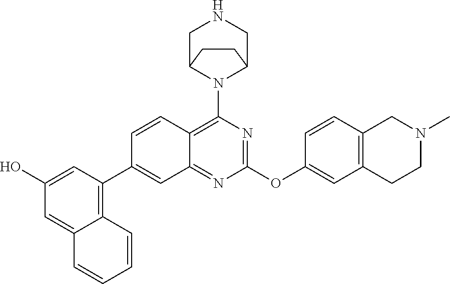 [SMILES: CN1CCc2cc(Oc3nc(N4C5CCC4CNC5)c4ccc(-c5cc(O)cc6ccccc56)cc4n3)ccc2C1] | 4-(4-((1R,5S)-3,8- diazabicyclo[3.2.1]octan-8-yl)-2- ((2-methyl-1,2,3,4- tetrahydroisoquinolin-6- yl)oxy)quinazolin-7-yl)naphthalen- 2-ol | 544.2 |
| P-0239 | 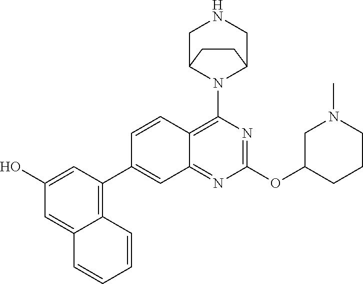 [SMILES: CN1CCCC(Oc2nc(N3C4CCC3CNC4)c3ccc(-c4cc(O)cc5ccccc45)cc3n2)C1] | 4-(4-((1R,5S)-3,8- diazabicyclo[3.2.1]octan-8-yl)-2- ((1-methylpiperidin-3- yl)oxy)quinazolin-7-yl)naphthalen- 2-ol | 496.2 |
| P-0240 | 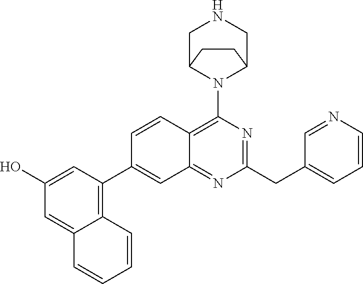 [SMILES: Oc1cc(-c2ccc3c(N4C5CCC4CNC5)nc(Cc4cccnc4)nc3c2)c2ccccc2c1] | 4-(4-((1R,5S)-3,8- diazabicyclo[3.2.1]octan-8-yl)-2- (pyridin-3-ylmethyl)quinazolin-7- yl)naphthalen-2-ol | 474.2 |
| P-0241 | 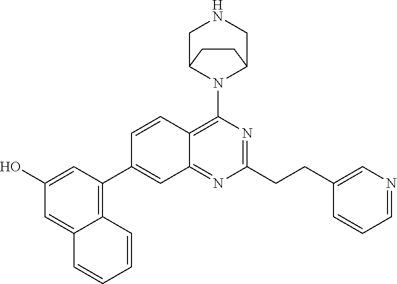 [SMILES: Oc1cc(-c2ccc3c(N4C5CCC4CNC5)nc(CCc4cccnc4)nc3c2)c2ccccc2c1] | 4-(4-((1R,5S)-3,8- diazabicyclo[3.2.1]octan-8-yl)-2-(2- (pyridin-3-yl)ethyl)quinazolin-7- yl)naphthalen-2-ol | 488.2 |
| P-0242 | 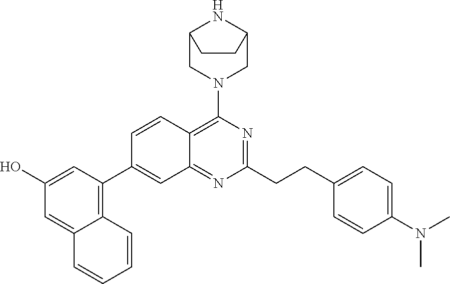 [SMILES: CN(C)c1ccc(CCc2nc(N3CC4CCC(C3)N4)c3ccc(-c4cc(O)cc5ccccc45)cc3n2)cc1] | 4-(4-((1R,5S)-3,8- diazabicyclo[3.2.1]octan-3-yl)-2-(4- (dimethylamino)phenethyl)quinazo- lin-7-yl)naphthalen-2-ol | 530.2 |
| P-0243 | 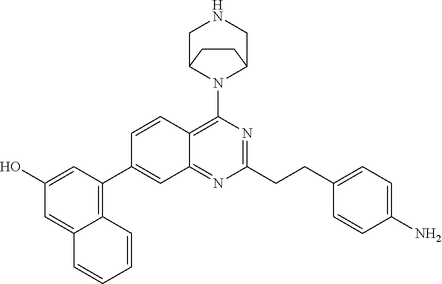 [SMILES: Nc1ccc(CCc2nc(N3C4CCC3CNC4)c3ccc(-c4cc(O)cc5ccccc45)cc3n2)cc1] | 4-(2-(4-aminophenethyl)-4- ((1R,5S)-3,8- diazabicyclo[3.2.1]octan-8- yl)quinazolin-7-yl)naphthalen-2-ol | 502.2 |
| P-0244 | 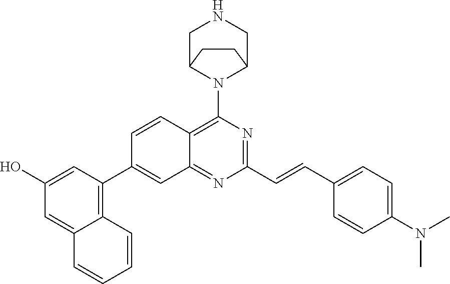 [SMILES: CN(C)c1ccc(/C=C/c2nc(N3C4CCC3CNC4)c3ccc(-c4cc(O)cc5ccccc45)cc3n2)cc1] | 4-(4-((1R,5S)-3,8- diazabicyclo[3.2.1]octan-8-yl)-2- ((E)-4- (dimethylamino)styryl)quinazolin- 7-yl)naphthalen-2-ol | 500.2 |
| P-0245 | 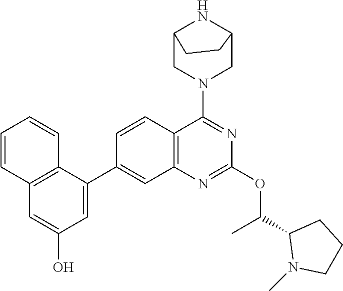 [SMILES: CC(Oc1nc(N2CC3CCC(C2)N3)c2ccc(-c3cc(O)cc4ccccc34)cc2n1)[C@@H]1CCCN1C] | 4-(4-((1R,5S)-3,8- diazabicyclo[3.2.1]octan-3-yl)-2- ((R)-1-((S)-1-methylpyrrolidin-2- yl)ethoxy)quinazolin-7- yl)naphthalen-2-ol | 510.2 |
| P-0246 | 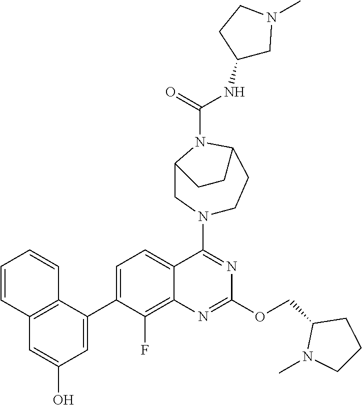 [SMILES: CN1CC[C@@H](NC(=O)N2C3CCC2CN(c2nc(OC[C@@H]4CCCN4C)nc4c(F)c(-c5cc(O)cc6ccccc56)ccc24)CC3)C1] | (1R,6S)-3-(8-fluoro-7-(3- hydroxynaphthalen-1-yl)-2-(((S)-1- methylpyrrolidin-2- yl)methoxy)quinazolin-4-yl)-N- ((R)-1-methylpyrrolidin-3-yl)-3,9- diazabicyclo[4.2.1]nonane-9- carboxamide | 654.3 |
| P-0247 | 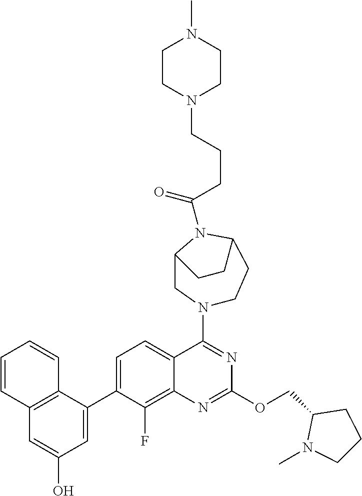 [SMILES: CN1CCN(CCCC(=O)N2C3CCC2CN(c2nc(OC[C@@H]4CCCN4C)nc4c(F)c(-c5cc(O)cc6ccccc56)ccc24)CC3)CC1] | 1-(3-(8-fluoro-7-(3- hydroxynaphthalen-1-yl)-2-(((S)-1- methylpyrrolidin-2- yl)methoxy)quinazolin-4-yl)-3,9- diazabicyclo[4.2.1]nonan-9-yl)-4- (4-methylpiperazin-1-yl)butan-1- one | 696.3 |
| P-0248 | 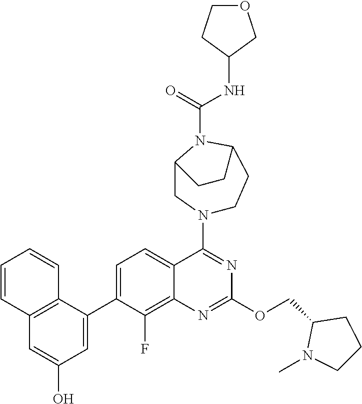 [SMILES: CN1CCC[C@H]1COc1nc(N2CCC3CCC(C2)N3C(=O)NC2CCOC2)c2ccc(-c3cc(O)cc4ccccc34)c(F)c2n1] | (1R,6 S)-3-(8-fluoro-7-(3- hydroxynaphthalen-1-yl)-2-(((S)-1- methylpyrrolidin-2- yl)methoxy)quinazolin-4-yl)-N- (tetrahydrofuran-3-yl)-3,9- diazabicyclo[4.2.1]nonane-9- carboxamide | 641.7 |
| P-0249 | 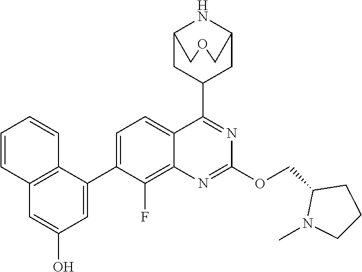 [SMILES: CN1CCC[C@H]1COc1nc(C2CC3COCC(C2)N3)c2ccc(-c3cc(O)cc4ccccc34)c(F)c2n1] | 4-(4-(3-oxa-7,9- diazabicyclo[3.3.1]nonan-7-yl)-8- fluoro-2-(((S)-1-methylpyrrolidin- 2-yl)methoxy)quinazolin-7- yl)naphthalen-2-ol | 530.2 |
| P-0250 | 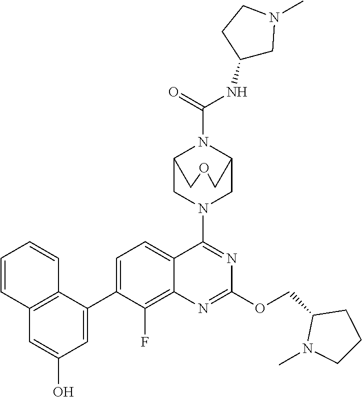 [SMILES: CN1CC[C@@H](NC(=O)N2C3COCC2CN(c2nc(OC[C@@H]4CCCN4C)nc4c(F)c(-c5cc(O)cc6ccccc56)ccc24)C3)C1] | 7-(8-fluoro-7-(3- hydroxynaphthalen-1-yl)-2-(((S)-1- methylpyrrolidin-2- yl)methoxy)quinazolin-4-yl)-N- ((R)-1-methylpyrrolidin-3-yl)-3- oxa-7,9-diazabicyclo[3.3.1]nonane- 9-carboxamide | 656.3 |
| P-0251 | 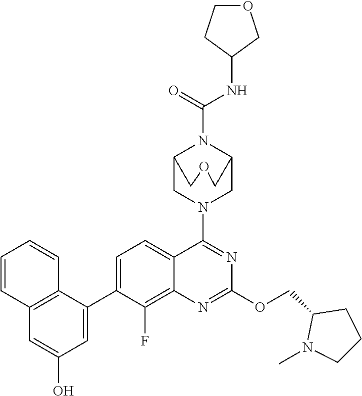 [SMILES: CN1CCC[C@H]1COc1nc(N2CC3COCC(C2)N3C(=O)NC2CCOC2)c2ccc(-c3cc(O)cc4ccccc34)c(F)c2n1] | rac-7-(8-fluoro-7-(3- hydroxynaphthalen-1-yl)-2-(((R)-1- methylpyrrolidin-2- yl)methoxy)quinazolin-4-yl)-N- (tetrahydrofuran-3-yl)-3-oxa-7,9- diazabicyclo[3.3.1]nonane-9- carboxamide | 643.7 |
| P-0252 | 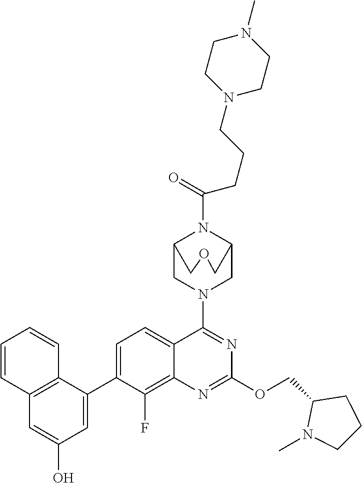 [SMILES: CN1CCN(CCCC(=O)N2C3COCC2CN(c2nc(OC[C@@H]4CCCN4C)nc4c(F)c(-c5cc(O)cc6ccccc56)ccc24)C3)CC1] | 1-(7-(8-fluoro-7-(3- hydroxynaphthalen-1-yl)-2-(((S)-1- methylpyrrolidin-2- yl)methoxy)quinazolin-4-yl)-3-oxa- 7,9-diazabicyclo[3.3.1]nonan-9-yl)- 4-(4-methylpiperazin-1-yl)butan-1- one | 698.4 |
| P-0253 | 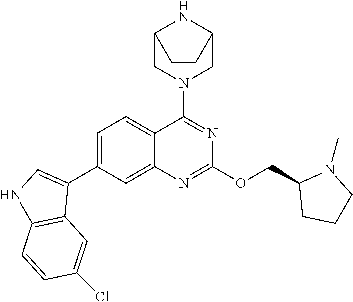 [SMILES: CN1CCC[C@H]1COc1nc(N2CC3CCC(C2)N3)c2ccc(-c3c[nH]c4ccc(Cl)cc34)cc2n1] | 4-((1R,5S)-3,8- diazabicyclo[3.2.1]octan-3-yl)-7- (5-chloro-1H-indol-3-yl)-2-(((S)-1- methylpyrrolidin-2- yl)methoxy)quinazoline | 503.2 |
| P-0254 | 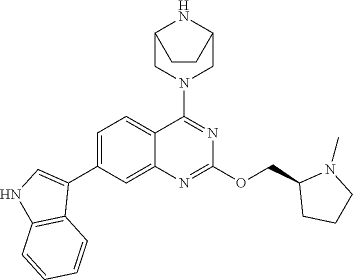 [SMILES: CN1CCC[C@H]1COc1nc(N2CC3CCC(C2)N3)c2ccc(-c3c[nH]c4ccccc34)cc2n1] | 4-((1R,5S)-3,8- diazabicyclo[3.2.1]octan-3-yl)-7- (1H-indol-3-yl)-2-(((S)-1- methylpyrrolidin-2- yl)methoxy)quinazoline | 469.6 |
| P-0255 | 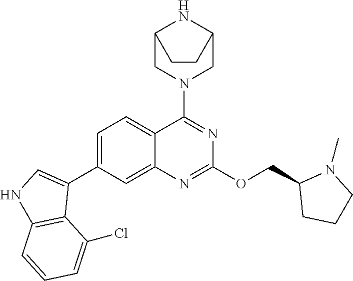 [SMILES: CN1CCC[C@H]1COc1nc(N2CC3CCC(C2)N3)c2ccc(-c3c[nH]c4cccc(Cl)c34)cc2n1] | 4-((1R,5S)-3,8- diazabicyclo[3.2.1]octan-3-yl)-7- (4-chloro-1H-indol-3-yl)-2-(((S)-1- methylpyrrolidin-2- yl)methoxy)quinazoline | 503.2 |
| P-0256 | 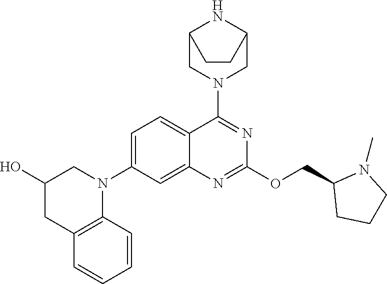 [SMILES: CN1CCC[C@H]1COc1nc(N2CC3CCC(C2)N3)c2ccc(N3CC(O)Cc4ccccc43)cc2n1] | 1-(4-((1R,5S)-3,8- diazabicyclo[3.2.1]octan-3-yl)-2- (((RS)-1-methylpyrrolidin-2- yl)methoxy)quinazolin-7-yl)- 1,2,3,4-tetrahydroquinolin-3-ol | 501.3 |
| P-0257 | 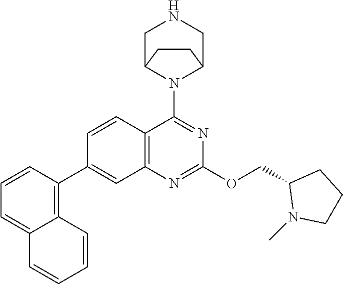 [SMILES: CN1CCC[C@H]1COc1nc(N2C3CCC2CNC3)c2ccc(-c3cccc4ccccc34)cc2n1] | 4-((1R,5S)-3,8- diazabicyclo[3.2.1]octan-8-yl)-2- (((S)-1-methylpyrrolidin-2- yl)methoxy)-7-(naphthalen-1- yl)quinazoline | 480.4 |
| P-0258 | 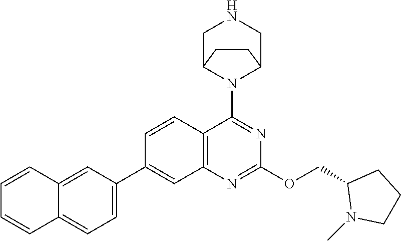 [SMILES: CN1CCC[C@H]1COc1nc(N2C3CCC2CNC3)c2ccc(-c3ccc4ccccc4c3)cc2n1] | 4-((1R,5S)-3,8- diazabicyclo[3.2.1]octan-8-yl)-2- (((S)-1-methylpyrrolidin-2- yl)methoxy)-7-(naphthalen-2- yl)quinazoline | 480.4 |
| P-0259 | 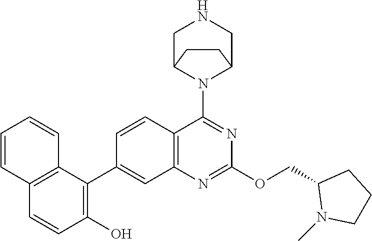 [SMILES: CN1CCC[C@H]1COc1nc(N2C3CCC2CNC3)c2ccc(-c3c(O)ccc4ccccc34)cc2n1] | 1-(4-((1R,5S)-3,8- diazabicyclo[3.2.1]octan-8-yl)-2- (((S)-1-methylpyrrolidin-2- yl)methoxy)quinazolin-7- yl)naphthalen-2-ol | 496.3 |
| P-0260 | 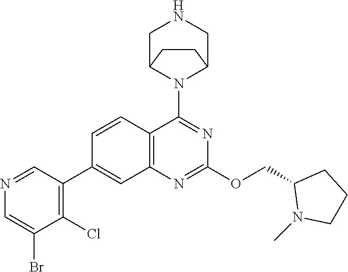 [SMILES: CN1CCC[C@H]1COc1nc(N2C3CCC2CNC3)c2ccc(-c3cncc(Br)c3Cl)cc2n1] | 4-((1R,5S)-3,8- diazabicyclo[3.2.1]octan-8-yl)-7- (5-bromo-4-chloropyridin-3-yl)-2- (((S)-1-methylpyrrolidin-2- yl)methoxy)quinazoline | 543.2 |
| P-0261 | 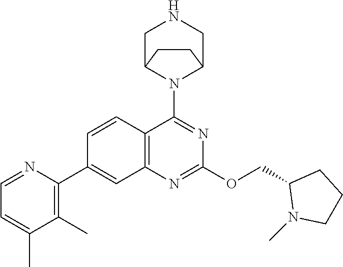 [SMILES: Cc1ccnc(-c2ccc3c(N4C5CCC4CNC5)nc(OC[C@@H]4CCCN4C)nc3c2)c1C] | 4-((1R,5S)-3,8- diazabicyclo[3.2.1]octan-8-yl)-7- (3,4-dimethylpyridin-2-yl)-2-(((S)- 1-methylpyrrolidin-2- yl)methoxy)quinazoline | 459.4 |
| P-0262 | 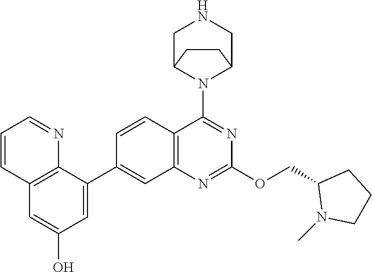 [SMILES: CN1CCC[C@H]1COc1nc(N2C3CCC2CNC3)c2ccc(-c3cc(O)cc4cccnc34)cc2n1] | 8-(4-((1R,5S)-3,8- diazabicyclo[3.2.1]octan-8-yl)-2- (((S)-1-methylpyrrolidin-2- yl)methoxy)quinazolin-7- yl)quinolin-6-ol | 497.4 |
| P-0263 | 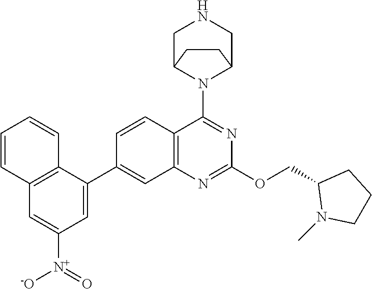 | 4-((1R,5S)-3,8- diazabicyclo[3.2.1]octan-8-yl)-2- (((S)-1-methylpyrrolidin-2- yl)methoxy)-7-(3-nitronaphthalen- 1-yl)quinazoline | 525.3 |
| P-0264 | 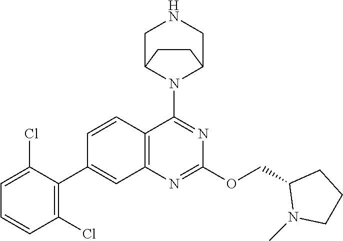 [SMILES: CN1CCC[C@H]1COc1nc(N2C3CCC2CNC3)c2ccc(-c3c(Cl)cccc3Cl)cc2n1] | 4-((1R,5S)-3,8- diazabicyclo[3.2.1]octan-8-yl)-7- (2,6-dichlorophenyl)-2-(((S)-1- methylpyrrolidin-2- yl)methoxy)quinazoline | 498.3 |
| P-0265 | 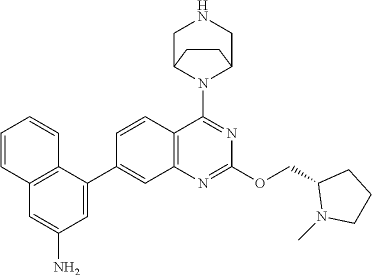 [SMILES: CN1CCC[C@H]1COc1nc(N2C3CCC2CNC3)c2ccc(-c3cc(N)cc4ccccc34)cc2n1] | 4-(4-((1R,5S)-3,8- diazabicyclo[3.2.1]octan-8-yl)-2- (((S)-1-methylpyrrolidin-2- yl)methoxy)quinazolin-7- yl)naphthalen-2-amine | 495.3 |
| P-0266 | 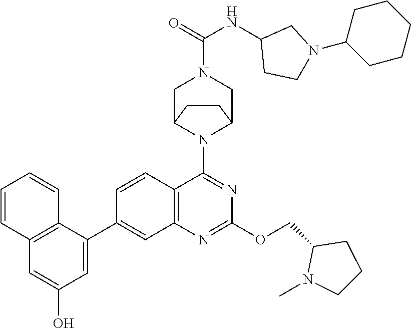 [SMILES: CN1CCC[C@H]1COc1nc(N2C3CCC2CN(C(=O)NC2CCN(C4CCCCC4)C2)C3)c2ccc(-c3cc(O)cc4ccccc34)cc2n1] | (1R,5S)-N-(1-cyclohexylpyrrolidin- 3-yl)-8-(7-(3-hydroxynaphthalen-1- yl)-2-(((S)-1-methylpyrrolidin-2- yl)methoxy)quinazolin-4-yl)-3,8- diazabicyclo[3.2.1]octane-3- carboxamide | 690.4 |
| P-0267 | 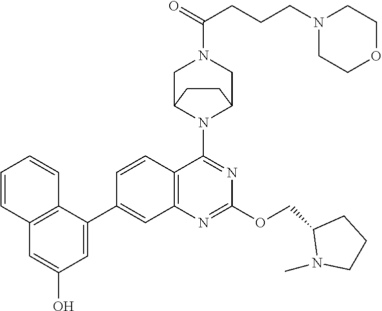 [SMILES: CN1CCC[C@H]1COc1nc(N2C3CCC2CN(C(=O)CCCN2CCOCC2)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-4- morpholinobutan-1-one | 651.7 |
| P-0268 | 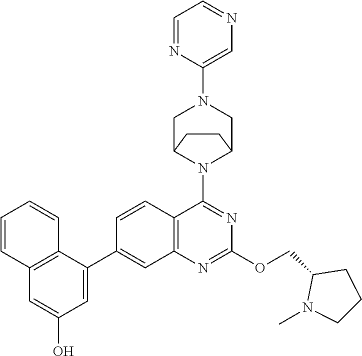 [SMILES: CN1CCC[C@H]1COc1nc(N2C3CCC2CN(c2cnccn2)C3)c2ccc(-c3cc(O)cc4ccccc34)cc2n1] | 4-(2-(((S)-1-methylpyrrolidin-2- yl)methoxy)-4-((1R,5S)-3-(pyrazin- 2-yl)-3,8-diazabicyclo[3.2.1]octan- 8-yl)quinazolin-7-yl)naphthalen-2- ol | 574.3 |
| P-0269 | 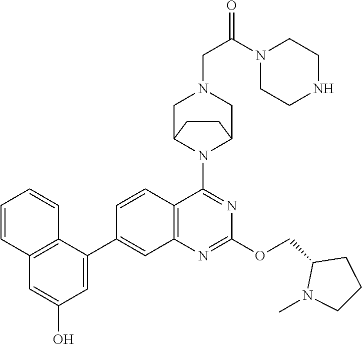 [SMILES: CN1CCC[C@H]1COc1nc(N2C3CCC2CN(CC(=O)N2CCNCC2)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-1- (piperazin-1-yl)ethan-1-one | 622.6 |
| P-0270 | 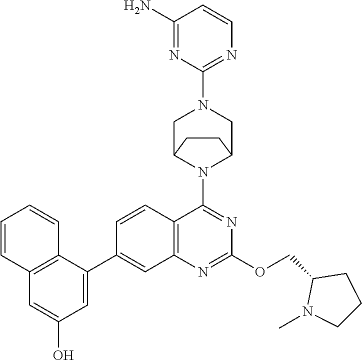 [SMILES: CN1CCC[C@H]1COc1nc(N2C3CCC2CN(c2nccc(N)n2)C3)c2ccc(-c3cc(O)cc4ccccc34)cc2n1] | 4-(4-((1R,5S)-3-(4- aminopyrimidin-2-yl)-3,8- diazabicyclo[3.2.1]octan-8-yl)-2- (((S)-1-methylpyrrolidin-2- yl)methoxy)quinazolin-7- yl)naphthalen-2-ol | 589.6 |
| P-0271 | 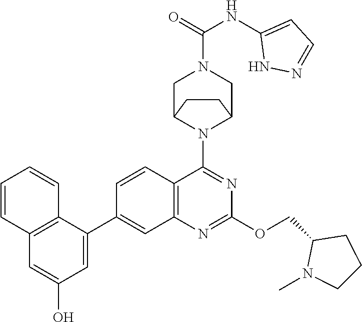 [SMILES: CN1CCC[C@H]1COc1nc(N2C3CCC2CN(C(=O)Nc2ccn[nH]2)C3)c2ccc(-c3cc(O)cc4ccccc34)cc2n1] | (1R,5S)-8-(7-(3- hydroxynaphthalen-1-yl)-2-(((S)-1- methylpyrrolidin-2- yl)methoxy)quinazolin-4-yl)-N- (1H-pyrazol-5-yl)-3,8- diazabicyclo[3.2.1]octane-3- carboxamide | 605.5 |
| P-0272 | 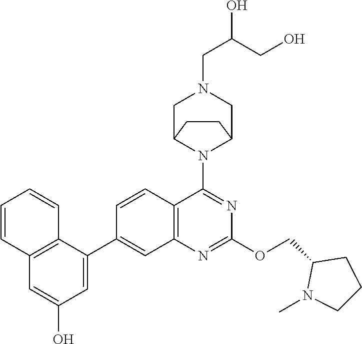 [SMILES: CN1CCC[C@H]1COc1nc(N2C3CCC2CN(CC(O)CO)C3)c2ccc(-c3cc(O)cc4ccccc34)cc2n1] | 3-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)propane-1,2-diol | 570.4 |
| P-0273 | 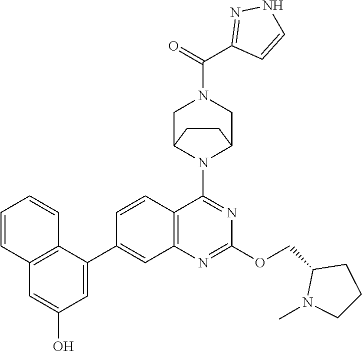 [SMILES: CN1CCC[C@H]1COc1nc(N2C3CCC2CN(C(=O)c2cc[nH]n2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)(1H- pyrazol-3-yl)methanone | 590.5 |
| P-0274 | 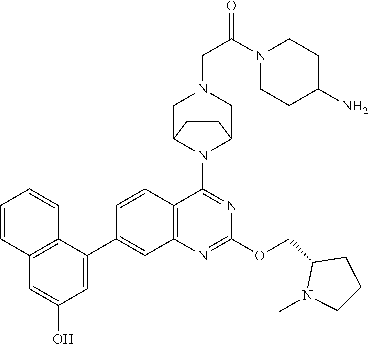 [SMILES: CN1CCC[C@H]1COc1nc(N2C3CCC2CN(CC(=O)N2CCC(N)CC2)C3)c2ccc(-c3cc(O)cc4ccccc34)cc2n1] | 1-(4-aminopiperidin-1-yl)-2- ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)ethan-1-one | 636.7 |
| P-0275 | 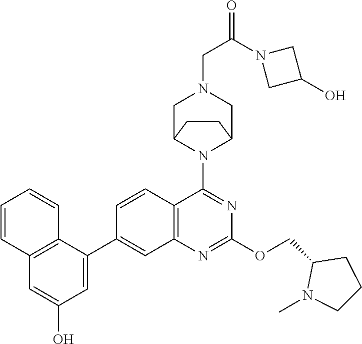 [SMILES: CN1CCC[C@H]1COc1nc(N2C3CCC2CN(CC(=O)N2CC(O)C2)C3)c2ccc(-c3cc(O)cc4ccccc34)cc2n1] | 1-(3-hydroxyazetidin-1-yl)-2- ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)ethan-1-one | 609.4 |
| P-0276 | 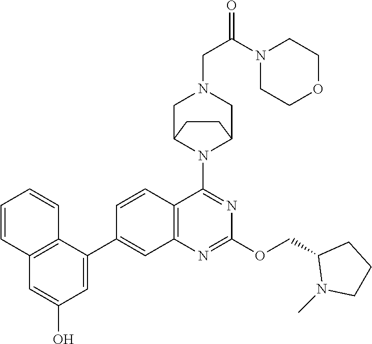 [SMILES: CN1CCC[C@H]1COc1nc(N2C3CCC2CN(CC(=O)N2CCOCC2)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-1- morpholinoethan-1-one | 623.5 |
| P-0277 | 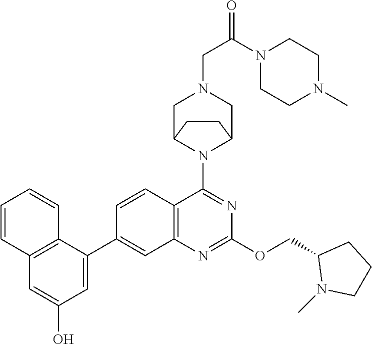 [SMILES: CN1CCN(C(=O)CN2CC3CCC(C2)N3c2nc(OC[C@@H]3CCCN3C)nc3cc(-c4cc(O)cc5ccccc45)ccc23)CC1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-1- (4-methylpiperazin-1-yl)ethan-1- one | 636.7 |
| P-0278 | 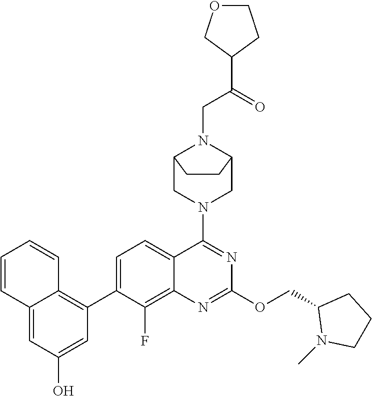 [SMILES: CN1CCC[C@H]1COc1nc(N2CC3CCC(C2)N3CC(=O)C2CCOC2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-1- (tetrahydrofuran-3-yl)ethan-1-one | 626.3 |
| P-0279 | 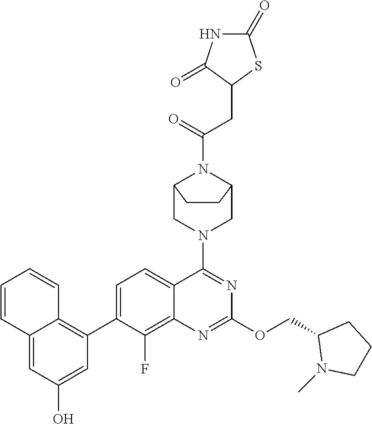 [SMILES: CN1CCC[C@H]1COc1nc(N2CC3CCC(C2)N3C(=O)CC2SC(=O)NC2=O)c2ccc(-c3cc(O)cc4ccccc34)c(F)c2n1] | 5-(2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethyl)thiazolidine-2,4-dione | 671.2 |
| P-0280 | 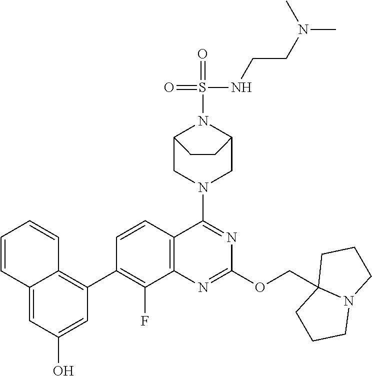 [SMILES: CN(C)CCNS(=O)(=O)N1C2CCC1CN(c1nc(OCC34CCCN3CCC4)nc3c(F)c(-c4cc(O)cc5ccccc45)ccc13)C2] | (1R,5S)-N-(2- (dimethylamino)ethyl)-3-(8-fluoro- 7-(3-hydroxynaphthalen-1-yl)-2- ((tetrahydro-1H-pyrrolizin-7a(5H)- yl)methoxy)quinazolin-4-yl)-3,8- diazabicyclo[3.2.1]octane-8- sulfonamide | 690.5 |
| P-0281 | 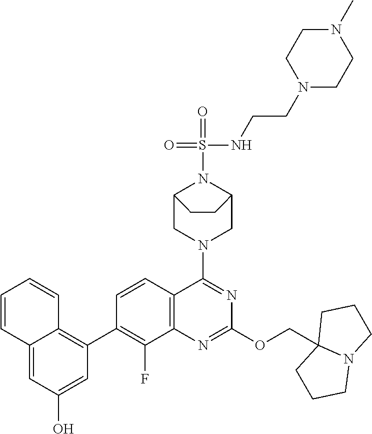 [SMILES: CN1CCN(CCNS(=O)(=O)N2C3CCC2CN(c2nc(OCC45CCCN4CCC5)nc4c(F)c(-c5cc(O)cc6ccccc56)ccc24)C3)CC1] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-N-(2- (4-methylpiperazin-1-yl)ethyl)-3,8- diazabicyclo[3.2.1]octane-8- sulfonamide | 745.5 |
| P-0282 | 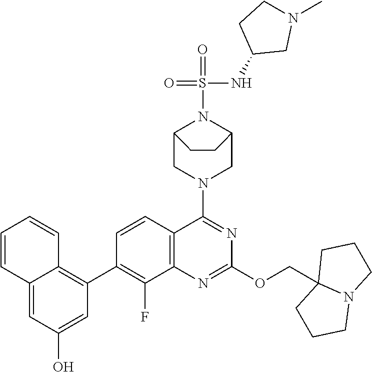 [SMILES: CN1CC[C@@H](NS(=O)(=O)N2C3CCC2CN(c2nc(OCC45CCCN4CCC5)nc4c(F)c(-c5cc(O)cc6ccccc56)ccc24)C3)C1] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-N- ((R)-1-methylpyrrolidin-3-yl)-3,8- diazabicyclo[3.2.1]octane-8- sulfonamide | 702.5 |
| P-0283 | 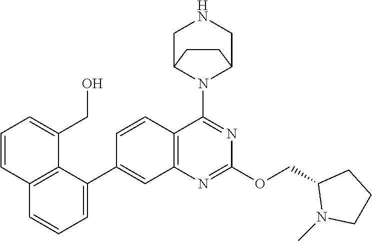 [SMILES: CN1CCC[C@H]1COc1nc(N2C3CCC2CNC3)c2ccc(-c3cccc4cccc(CO)c34)cc2n1] | (8-(4-((1R,5S)-3,8- diazabicyclo[3.2.1]octan-8-yl)-2- (((S)-1-methylpyrrolidin-2- yl)methoxy)quinazolin-7- yl)naphthalen-1-yl)methanol | 510.4 |
| P-0284 | 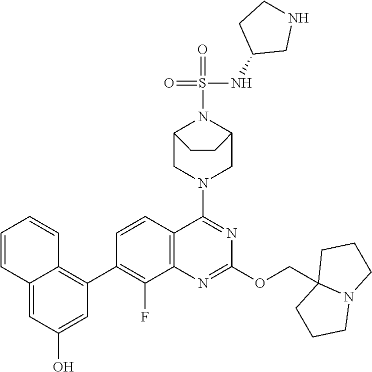 [SMILES: O=S(=O)(N[C@@H]1CCNC1)N1C2CCC1CN(c1nc(OCC34CCCN3CCC4)nc3c(F)c(-c4cc(O)cc5ccccc45)ccc13)C2] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-N- ((R)-pyrrolidin-3-yl)-3,8- diazabicyclo[3.2.1]octane-8- sulfonamide | 689.4 |
| P-0285 | 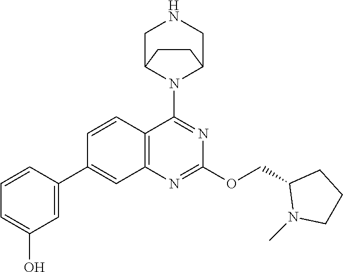 [SMILES: CN1CCC[C@H]1COc1nc(N2C3CCC2CNC3)c2ccc(-c3cccc(O)c3)cc2n1] | 3-(4-((1R,5S)-3,8- diazabicyclo[3.2.1]octan-8-yl)-2- (((S)-1-methylpyrrolidin-2- yl)methoxy)quinazolin-7-yl)phenol | 446.3 |
| P-0286 | 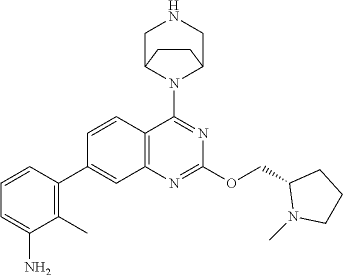 [SMILES: Cc1c(N)cccc1-c1ccc2c(N3C4CCC3CNC4)nc(OC[C@@H]3CCCN3C)nc2c1] | 3-(4-((1R,5S)-3,8- diazabicyclo[3.2.1]octan-8-yl)-2- (((S)-1-methylpyrrolidin-2- yl)methoxy)quinazolin-7-yl)-2- methylaniline | 459.4 |
| P-0287 | 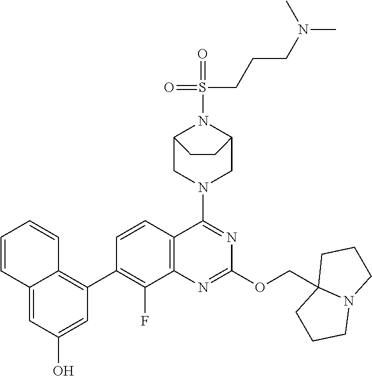 [SMILES: CN(C)CCCS(=O)(=O)N1C2CCC1CN(c1nc(OCC34CCCN3CCC4)nc3c(F)c(-c4cc(O)cc5ccccc45)ccc13)C2] | 4-(4-((1R,5S)-8-((3- (dimethylamino)propyl)sulfonyl)- 3,8-diazabicyclo[3.2.1]octan-3-yl)- 8-fluoro-2-((tetrahydro-1H- pyrrolizin-7a(5H)- yl)methoxy)quinazolin-7- yl)naphthalen-2-ol | 689.5 |
| P-0288 | 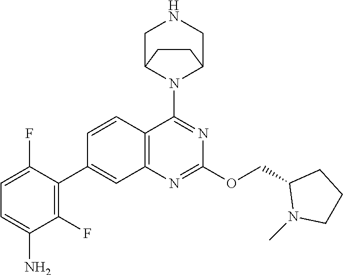 [SMILES: CN1CCC[C@H]1COc1nc(N2C3CCC2CNC3)c2ccc(-c3c(F)ccc(N)c3F)cc2n1] | 3-(4-((1R,5S)-3,8- diazabicyclo[3.2.1]octan-8-yl)-2- (((S)-1-methylpyrrolidin-2- yl)methoxy)quinazolin-7-yl)-2,4- difluoroaniline | 481.4 |
| P-0289 | 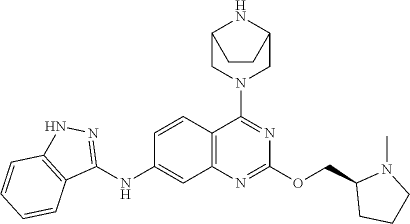 [SMILES: CN1CCC[C@H]1COc1nc(N2CC3CCC(C2)N3)c2ccc(Nc3n[nH]c4ccccc34)cc2n1] | 4-((1R,5S)-3,8- diazabicyclo[3.2.1]octan-3-yl)-N- (1H-indazol-3-yl)-2-(((S)-1- methylpyrrolidin-2- yl)methoxy)quinazolin-7-amine | 485.3 |
| P-0290 | 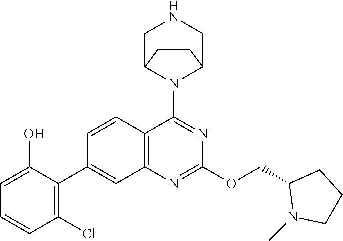 [SMILES: CN1CCC[C@H]1COc1nc(N2C3CCC2CNC3)c2ccc(-c3c(O)cccc3Cl)cc2n1] | 2-(4-((1R,5S)-3,8- diazabicyclo[3.2.1]octan-8-yl)-2- (((S)-1-methylpyrrolidin-2- yl)methoxy)quinazolin-7-yl)-3- chlorophenol | 480.3 |
| P-0291 | 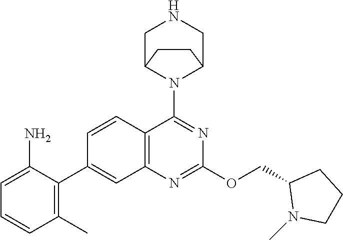 [SMILES: Cc1cccc(N)c1-c1ccc2c(N3C4CCC3CNC4)nc(OC[C@@H]3CCCN3C)nc2c1] | 2-(4-((1R,5S)-3,8- diazabicyclo[3.2.1]octan-8-yl)-2- (((S)-1-methylpyrrolidin-2- yl)methoxy)quinazolin-7-yl)-3- methylaniline | 459.3 |
| P-0292 | 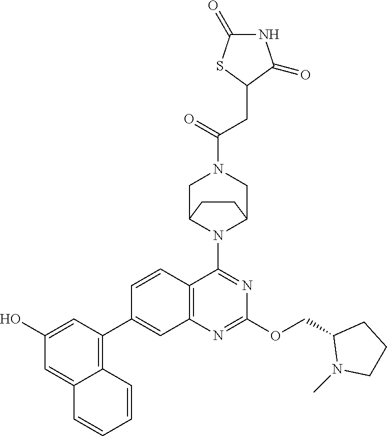 [SMILES: CN1CCC[C@H]1COc1nc(N2C3CCC2CN(C(=O)CC2SC(=O)NC2=O)C3)c2ccc(-c3cc(O)cc4ccccc34)cc2n1] | 5-(2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxoethyl)thiazolidine-2,4-dione | 653.3 |
| P-0293 | 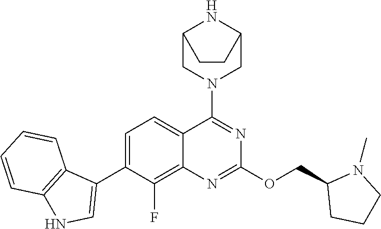 [SMILES: CN1CCC[C@H]1COc1nc(N2CC3CCC(C2)N3)c2ccc(-c3c[nH]c4ccccc34)c(F)c2n1] | 4-((1R,5S)-3,8- diazabicyclo[3.2.1]octan-3-yl)-8- fluoro-7-(1H-indol-3-yl)-2-(((S)-1- methylpyrrolidin-2- yl)methoxy)quinazoline | 487.6 |
| P-0294 | 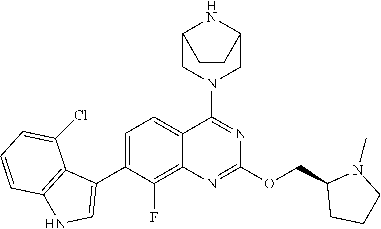 [SMILES: CN1CCC[C@H]1COc1nc(N2CC3CCC(C2)N3)c2ccc(-c3c[nH]c4cccc(Cl)c34)c(F)c2n1] | 4-((1R,5S)-3,8- diazabicyclo[3.2.1]octan-3-yl)-7- (4-chloro-1H-indol-3-yl)-8-fluoro- 2-(((S)-1-methylpyrrolidin-2- yl)methoxy)quinazoline | 521.2 |
| P-0295 | 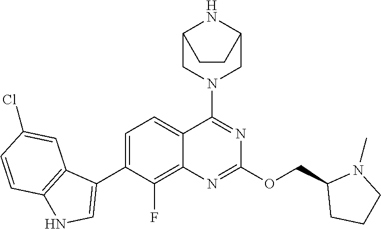 [SMILES: CN1CCC[C@H]1COc1nc(N2CC3CCC(C2)N3)c2ccc(-c3c[nH]c4ccc(Cl)cc34)c(F)c2n1] | 4-((1R,5S)-3,8- diazabicyclo[3.2.1]octan-3-yl)-7- (5-chloro-1H-indol-3-yl)-8-fluoro- 2-(((S)-1-methylpyrrolidin-2- yl)methoxy)quinazoline | 521.2 |
| P-0296 | 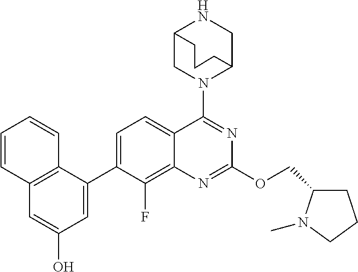 [SMILES: CN1CCC[C@H]1COc1nc(N2CC3CCCC2CN3)c2ccc(-c3cc(O)cc4ccccc34)c(F)c2n1] | 4-(4-(6,8-diazabicyclo[3.2.2]nonan- 6-yl)-8-fluoro-2-(((S)-1- methylpyrrolidin-2- yl)methoxy)quinazolin-7- yl)naphthalen-2-ol | 528.2 |
| P-0297 | 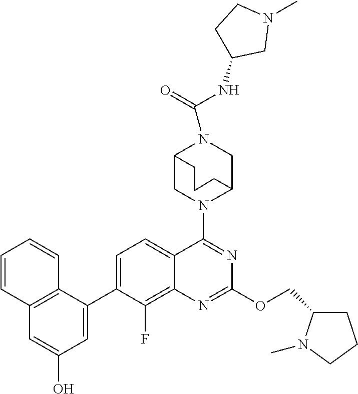 [SMILES: CN1CC[C@@H](NC(=O)N2CC3CCCC2CN3c2nc(OC[C@@H]3CCCN3C)nc3c(F)c(-c4cc(O)cc5ccccc45)ccc23)C1] | 8-(8-fluoro-7-(3- hydroxynaphthalen-1-yl)-2-(((S)-1- methylpyrrolidin-2- yl)methoxy)quinazolin-4-yl)-N- ((R)-1-methylpyrrolidin-3-yl)-6,8- diazabicyclo[3.2.2]nonane-6- carboxamide | 654.3 |
| P-0298 | 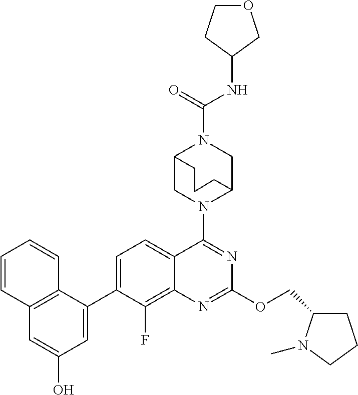 [SMILES: CN1CCC[C@H]1COc1nc(N2CC3CCCC2CN3C(=O)NC2CCOC2)c2ccc(-c3cc(O)cc4ccccc34)c(F)c2n1] | 8-(8-fluoro-7-(3- hydroxynaphthalen-1-yl)-2-(((S)-1- methylpyrrolidin-2- yl)methoxy)quinazolin-4-yl)-N- (tetrahydrofuran-3-yl)-6,8- diazabicyclo[3.2.2]nonane-6- carboxamide | 641.3 |
| P-0299 | 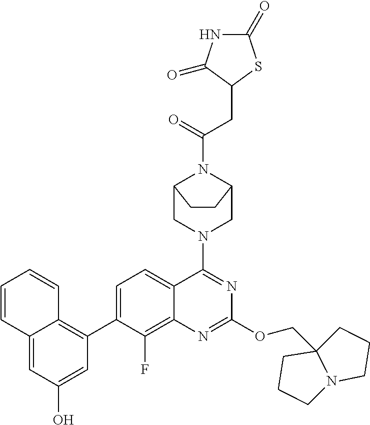 [SMILES: O=C1NC(=O)C(CC(=O)N2C3CCC2CN(c2nc(OCC45CCCN4CCC5)nc4c(F)c(-c5cc(O)cc6ccccc56)ccc24)C3)S1] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)thiazolidine-2,4-dione | 697.7 |
| P-0300 | 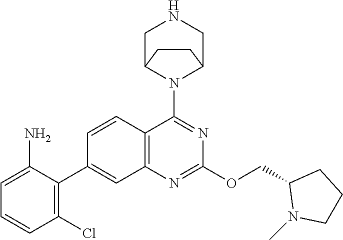 [SMILES: CN1CCC[C@H]1COc1nc(N2C3CCC2CNC3)c2ccc(-c3c(N)cccc3Cl)cc2n1] | 2-(4-((1R,5S)-3,8- diazabicyclo[3.2.1]octan-8-yl)-2- (((S)-1-methylpyrrolidin-2- yl)methoxy)quinazolin-7-yl)-3- chloroaniline | 479.3 |
| P-0301 | 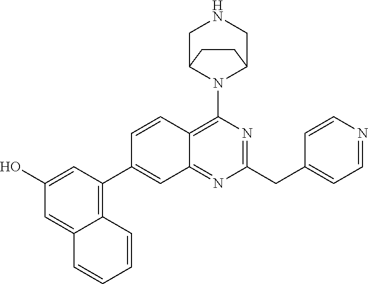 [SMILES: Oc1cc(-c2ccc3c(N4C5CCC4CNC5)nc(Cc4ccncc4)nc3c2)c2ccccc2c1] | 4-(4-((1R,5S)-3,8- diazabicyclo[3.2.1]octan-8-yl)-2- (pyridin-4-ylmethyl)quinazolin-7- yl)naphthalen-2-ol | 488.2 |
| P-0302 | 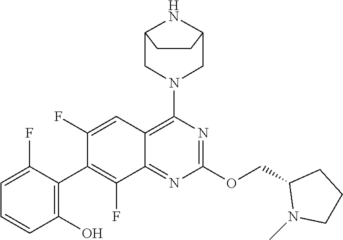 [SMILES: CN1CCC[C@H]1COc1nc(N2CC3CCC(C2)N3)c2cc(F)c(-c3c(O)cccc3F)c(F)c2n1] | 2-(4-((1R,5S)-3,8- diazabicyclo[3.2.1]octan-3-yl)-6,8- difluoro-2-(((S)-1- methylpyrrolidin-2- yl)methoxy)quinazolin-7-yl)-3- fluorophenol | 500.2 |
| P-0303 | 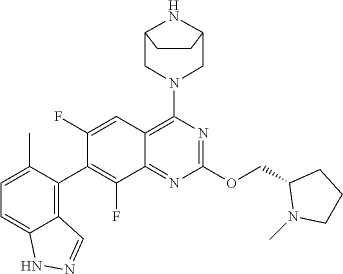 [SMILES: Cc1ccc2[nH]ncc2c1-c1c(F)cc2c(N3CC4CCC(C3)N4)nc(OC[C@@H]3CCCN3C)nc2c1F] | 4-((1R,5S)-3,8- diazabicyclo[3.2.1]octan-3-yl)-6,8- difluoro-7-(5-methyl-1H-indazol-4- yl)-2-(((S)-1-methylpyrrolidin-2- yl)methoxy)quinazoline | 520.2 |
| P-0304 | 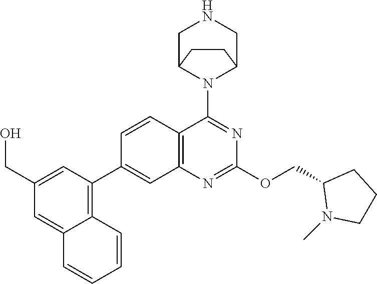 [SMILES: CN1CCC[C@H]1COc1nc(N2C3CCC2CNC3)c2ccc(-c3cc(CO)cc4ccccc34)cc2n1] | (4-(4-((1R,5S)-3,8- diazabicyclo[3.2.1]octan-8-yl)-2- (((S)-1-methylpyrrolidin-2- yl)methoxy)quinazolin-7- yl)naphthalen-2-yl)methanol | 510.3 |
| P-0305 | 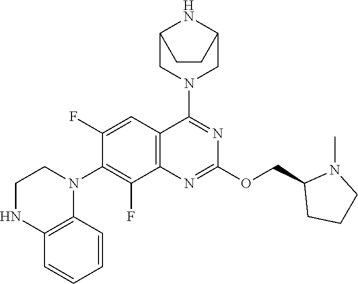 [SMILES: CN1CCC[C@H]1COc1nc(N2CC3CCC(C2)N3)c2cc(F)c(N3CCNc4ccccc43)c(F)c2n1] | 4-((1R,5S)-3,8- diazabicyclo[3.2.1]octan-3-yl)-7- (3,4-dihydroquinoxalin-1(2H)-yl)- 6,8-difluoro-2-(((S)-1- methylpyrrolidin-2- yl)methoxy)quinazoline | 522.4 |
| P-0306 | 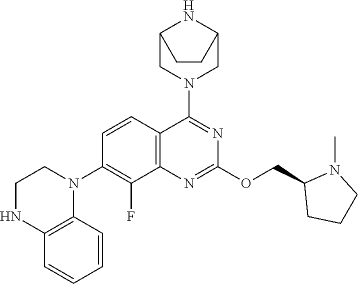 [SMILES: CN1CCC[C@H]1COc1nc(N2CC3CCC(C2)N3)c2ccc(N3CCNc4ccccc43)c(F)c2n1] | 4-((1R,5S)-3,8- diazabicyclo[3.2.1]octan-3-yl)-7- (3,4-dihydroquinoxalin-1(2H)-yl)- 8-fluoro-2-(((S)-1- methylpyrrolidin-2- yl)methoxy)quinazoline | 504.4 |
| P-0307 | 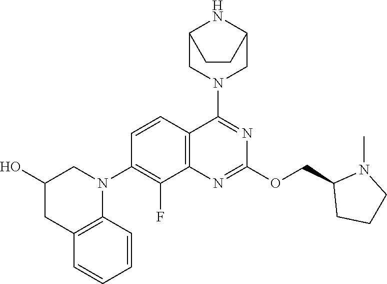 [SMILES: CN1CCC[C@H]1COc1nc(N2CC3CCC(C2)N3)c2ccc(N3CC(O)Cc4ccccc43)c(F)c2n1] | 1-(4-((1R,5S)-3,8- diazabicyclo[3.2.1]octan-3-yl)-8- fluoro-2-(((S)-1-methylpyrrolidin- 2-yl)methoxy)quinazolin-7-yl)- 1,2,3,4-tetrahydroquinolin-3-ol | 519.4 |
| P-0308 | 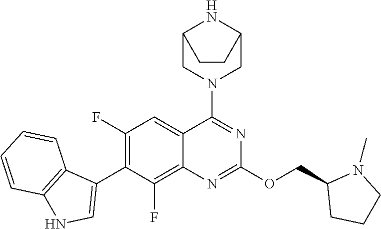 [SMILES: CN1CCC[C@H]1COc1nc(N2CC3CCC(C2)N3)c2cc(F)c(-c3c[nH]c4ccccc34)c(F)c2n1] | 4-((1R,5S)-3,8- diazabicyclo[3.2.1]octan-3-yl)-6,8- difluoro-7-(1H-indol-3-yl)-2-(((S)- 1-methylpyrrolidin-2- yl)methoxy)quinazoline | 505.2 |
| P-0309 | 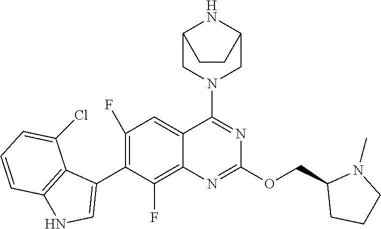 [SMILES: CN1CCC[C@H]1COc1nc(N2CC3CCC(C2)N3)c2cc(F)c(-c3c[nH]c4cccc(Cl)c34)c(F)c2n1] | 4-((1R,5S)-3,8- diazabicyclo[3.2.1]octan-3-yl)-7- (4-chloro-1H-indol-3-yl)-6,8- difluoro-2-(((S)-1- methylpyrrolidin-2- yl)methoxy)quinazoline | 539.2 |
| P-0310 | 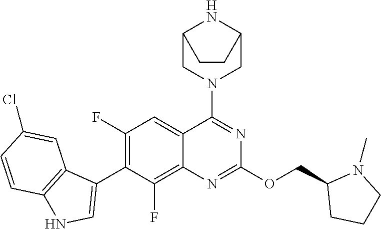 [SMILES: CN1CCC[C@H]1COc1nc(N2CC3CCC(C2)N3)c2cc(F)c(-c3c[nH]c4ccc(Cl)cc34)c(F)c2n1] | 4-((1R,5S)-3,8- diazabicyclo[3.2.1]octan-3-yl)-7- (5-chloro-1H-indol-3-yl)-6,8- difluoro-2-(((S)-1- methylpyrrolidin-2- yl)methoxy)quinazoline | 539.2 |
| P-0311 | 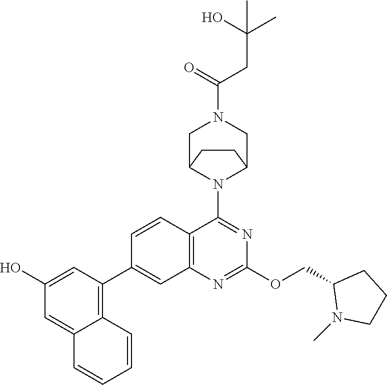 [SMILES: CN1CCC[C@H]1COc1nc(N2C3CCC2CN(C(=O)CC(C)(C)O)C3)c2ccc(-c3cc(O)cc4ccccc34)cc2n1] | 3-hydroxy-1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- methylbutan-1-one | 596.5 |
| P-0312 | 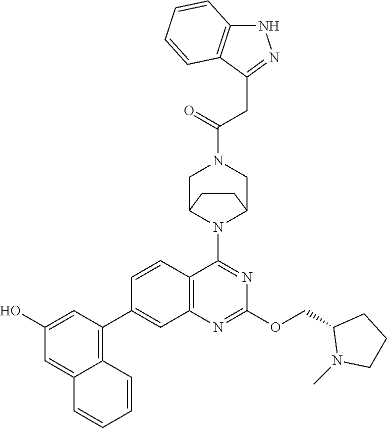 [SMILES: CN1CCC[C@H]1COc1nc(N2C3CCC2CN(C(=O)Cc2n[nH]c4ccccc24)C3)c2ccc(-c3cc(O)cc4ccccc34)cc2n1] | 1-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- (1H-indazol-3-yl)ethan-1-one | 654.4 |
| P-0313 | 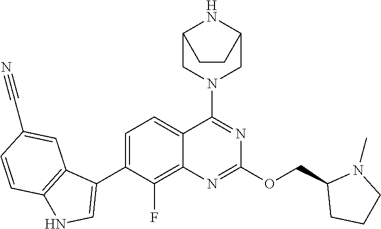 [SMILES: CN1CCC[C@H]1COc1nc(N2CC3CCC(C2)N3)c2ccc(-c3c[nH]c4ccc(C#N)cc34)c(F)c2n1] | 3-(4-((1R,5S)-3,8- diazabicyclo[3.2.1]octan-3-yl)-8- fluoro-2-(((S)-1-methylpyrrolidin- 2-yl)methoxy)quinazolin-7-yl)-1H- indole-5-carbonitrile | 512.2 |
| P-0314 | 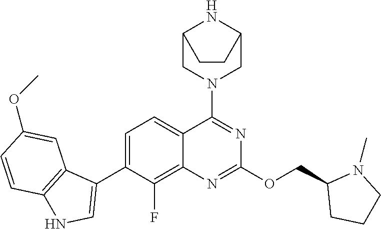 [SMILES: COc1ccc2[nH]cc(-c3ccc4c(N5CC6CCC(C5)N6)nc(OC[C@@H]5CCCN5C)nc4c3F)c2c1] | 4-((1R,5S)-3,8- diazabicyclo[3.2.1]octan-3-yl)-8- fluoro-7-(5-methoxy-1H-indol-3- yl)-2-(((S)-1-methylpyrrolidin-2- yl)methoxy)quinazoline | 517.2 |
| P-0315 | 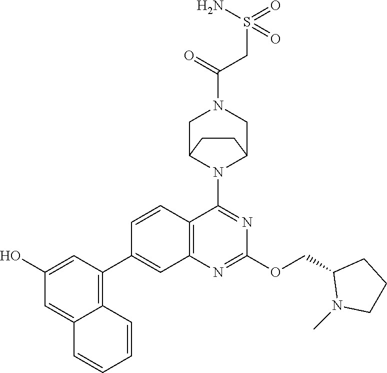 [SMILES: CN1CCC[C@H]1COc1nc(N2C3CCC2CN(C(=O)CS(N)(=O)=O)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxoethane-1-sulfonamide | 617.3 |
| P-0316 | 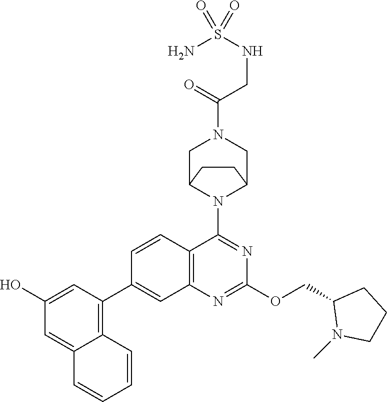 [SMILES: CN1CCC[C@H]1COc1nc(N2C3CCC2CN(C(=O)CNS(N)(=O)=O)C3)c2ccc(-c3cc(O)cc4ccccc34)cc2n1] | N-(2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxoethyl)aminosulfonamide | 632.4 |
| P-0317 | 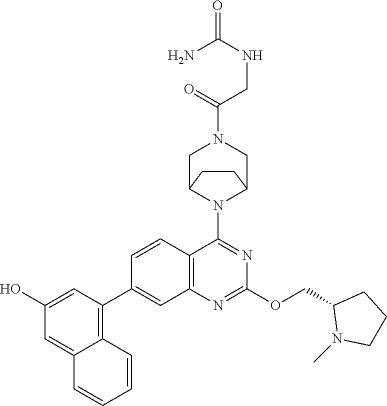 [SMILES: CN1CCC[C@H]1COc1nc(N2C3CCC2CN(C(=O)CNC(N)=O)C3)c2ccc(-c3cc(O)cc4ccccc34)cc2n1] | 1-(2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-2- oxoethyl)urea | 596.4 |
| P-0318 | 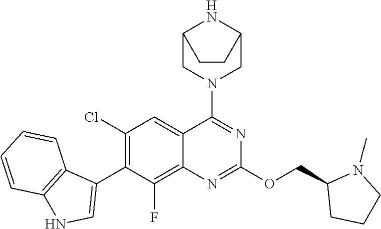 [SMILES: CN1CCC[C@H]1COc1nc(N2CC3CCC(C2)N3)c2cc(Cl)c(-c3c[nH]c4ccccc34)c(F)c2n1] | 4-((1R,5S)-3,8- diazabicyclo[3.2.1]octan-3-yl)-6- chloro-8-fluoro-7-(1H-indol-3-yl)- 2-(((S)-1-methylpyrrolidin-2- yl)methoxy)quinazoline | 521.2 |
| P-0319 | 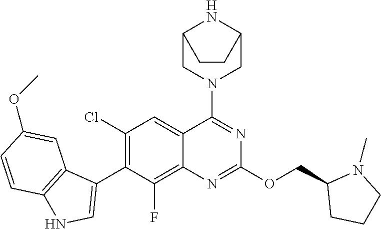 [SMILES: COc1ccc2[nH]cc(-c3c(Cl)cc4c(N5CC6CCC(C5)N6)nc(OC[C@@H]5CCCN5C)nc4c3F)c2c1] | 4-((1R,5S)-3,8- diazabicyclo[3.2.1]octan-3-yl)-6- chloro-8-fluoro-7-(5-methoxy-1H- indol-3-yl)-2-(((S)-1- methylpyrrolidin-2- yl)methoxy)quinazoline | 551.2 |
| P-0320 | 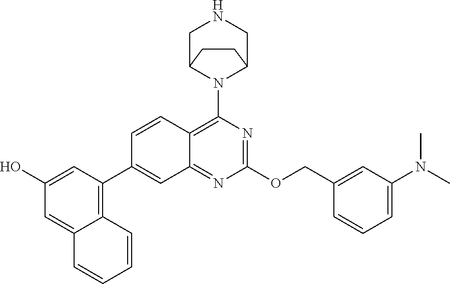 [SMILES: CN(C)c1cccc(COc2nc(N3C4CCC3CNC4)c3ccc(-c4cc(O)cc5ccccc45)cc3n2)c1] | 4-(4-((1R,5S)-3,8- diazabicyclo[3.2.1]octan-8-yl)-2- ((3- (dimethylamino)benzyl)oxy)quinaz- olin-7-yl)naphthalen-2-ol | 532.7 |
| P-0321 | 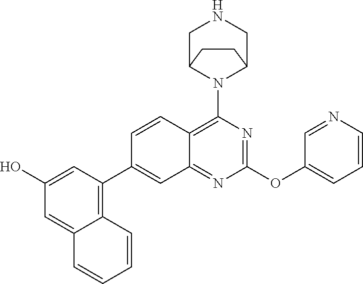 [SMILES: Oc1cc(-c2ccc3c(N4C5CCC4CNC5)nc(Oc4cccnc4)nc3c2)c2ccccc2c1] | 4-(4-((1R,5S)-3,8- diazabicyclo[3.2.1]octan-8-yl)-2- (pyridin-3-yloxy)quinazolin-7- yl)naphthalen-2-ol | 476.2 |
| P-0322 | 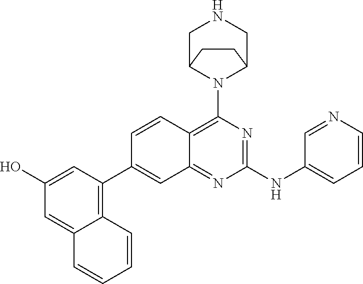 [SMILES: Oc1cc(-c2ccc3c(N4C5CCC4CNC5)nc(Nc4cccnc4)nc3c2)c2ccccc2c1] | 4-(4-((1R,5S)-3,8- diazabicyclo[3.2.1]octan-8-yl)-2- (pyridin-3-ylamino)quinazolin-7- yl)naphthalen-2-ol | 475.6 |
| P-0323 | 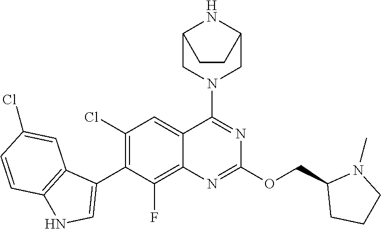 [SMILES: CN1CCC[C@H]1COc1nc(N2CC3CCC(C2)N3)c2cc(Cl)c(-c3c[nH]c4ccc(Cl)cc34)c(F)c2n1] | 4-((1R,5S)-3,8- diazabicyclo[3.2.1]octan-3-yl)-6- chloro-7-(5-chloro-1H-indol-3-yl)- 8-fluoro-2-(((S)-1- methylpyrrolidin-2- yl)methoxy)quinazoline | 555.1 |
| P-0324 | 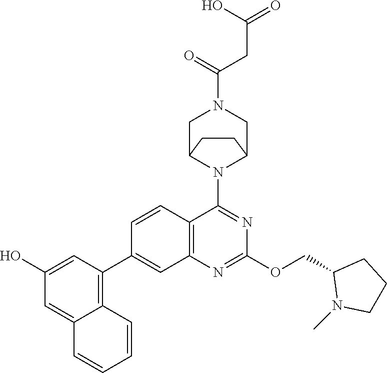 [SMILES: CN1CCC[C@H]1COc1nc(N2C3CCC2CN(C(=O)CC(=O)O)C3)c2ccc(-c3cc(O)cc4ccccc34)cc2n1] | 3-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- oxopropanoic acid | 582.4 |
| P-0325 | 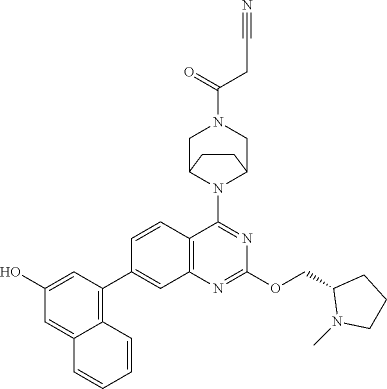 [SMILES: CN1CCC[C@H]1COc1nc(N2C3CCC2CN(C(=O)CC#N)C3)c2ccc(-c3cc(O)cc4ccccc34)cc2n1] | 3-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-3- oxopropanenitrile | 563.4 |
| P-0326 | 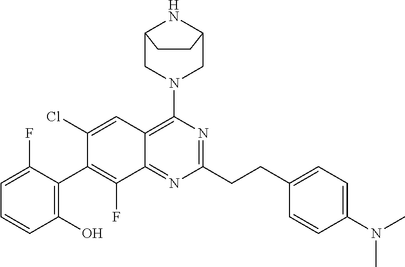 [SMILES: CN(C)c1ccc(CCc2nc(N3CC4CCC(C3)N4)c3cc(Cl)c(-c4c(O)cccc4F)c(F)c3n2)cc1] | 2-(4-((1R,5S)-3,8- diazabicyclo[3.2.1]octan-3-yl)-6- chloro-2-(4- (dimethylamino)phenethyl)-8- fluoroquinazolin-7-yl)-3- fluorophenol | 550.1 |
| P-0327 | 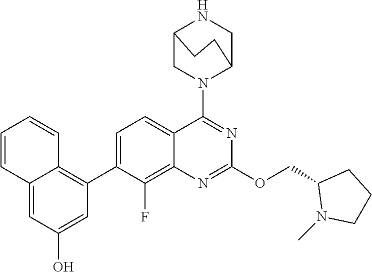 [SMILES: CN1CCC[C@H]1COc1nc(N2CC3CCC2CN3)c2ccc(-c3cc(O)cc4ccccc34)c(F)c2n1] | 4-(4-(2,5-diazabicyclo[2.2.2]octan- 2-yl)-8-fluoro-2-(((S)-1- methylpyrrolidin-2- yl)methoxy)quinazolin-7- yl)naphthalen-2-ol | 514.1 |
| P-0328 | 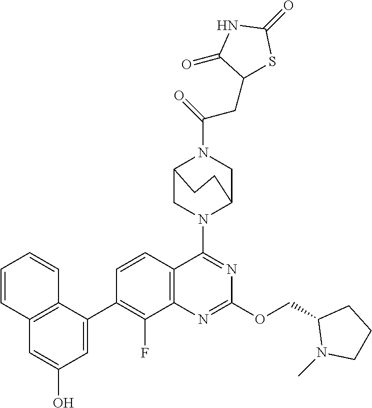 [SMILES: CN1CCC[C@H]1COc1nc(N2CC3CCC2CN3C(=O)CC2SC(=O)NC2=O)c2ccc(-c3cc(O)cc4ccccc34)c(F)c2n1] | 5-(2-(5-(8-fluoro-7-(3- hydroxynaphthalen-1-yl)-2-(((S)-1- methylpyrrolidin-2- yl)methoxy)quinazolin-4-yl)-2,5- diazabicyclo[2.2.2]octan-2-yl)-2- oxoethyl)thiazolidine-2,4-dione | 671.2 |
| P-0329 | 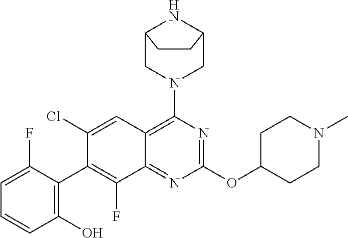 [SMILES: CN1CCC(Oc2nc(N3CC4CCC(C3)N4)c3cc(Cl)c(-c4c(O)cccc4F)c(F)c3n2)CC1] | 2-(4-((1R,5S)-3,8- diazabicyclo[3.2.1]octan-3-yl)-6- chloro-8-fluoro-2-((1- methylpiperidin-4- yl)oxy)quinazolin-7-yl)-3- fluorophenol | 515.1 |
| P-0330 | 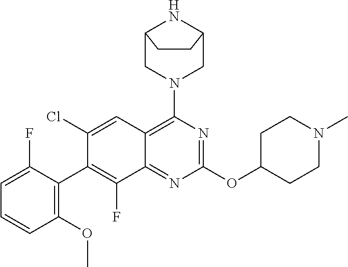 [SMILES: COc1cccc(F)c1-c1c(Cl)cc2c(N3CC4CCC(C3)N4)nc(OC3CCN(C)CC3)nc2c1F] | 4-((1R,5S)-3,8- diazabicyclo[3.2.1]octan-3-yl)-6- chloro-8-fluoro-7-(2-fluoro-6- methoxyphenyl)-2-((1- methylpiperidin-4- yl)oxy)quinazoline | 529.2 |
| P-0331 | 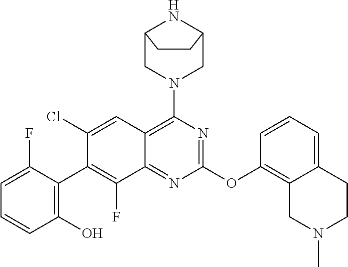 [SMILES: CN1CCc2cccc(Oc3nc(N4CC5CCC(C4)N5)c4cc(Cl)c(-c5c(O)cccc5F)c(F)c4n3)c2C1] | 2-(4-((1R,5S)-3,8- diazabicyclo[3.2.1]octan-3-yl)-6- chloro-8-fluoro-2-((2-methyl- 1,2,3,4-tetrahydroisoquinolin-8- yl)oxy)quinazolin-7-yl)-3- fluorophenol | 564.2 |
| P-0332 | 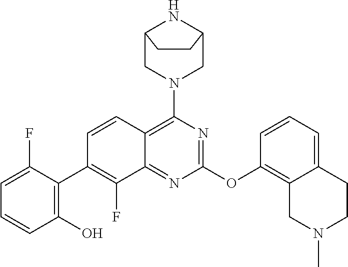 [SMILES: CN1CCc2cccc(Oc3nc(N4CC5CCC(C4)N5)c4ccc(-c5c(O)cccc5F)c(F)c4n3)c2C1] | 2-(4-((1R,5S)-3,8- diazabicyclo[3.2.1]octan-3-yl)-8- fluoro-2-((2-methyl-1,2,3,4- tetrahydroisoquinolin-8- yl)oxy)quinazolin-7-yl)-3- fluorophenol | 530.2 |
| P-0333 | 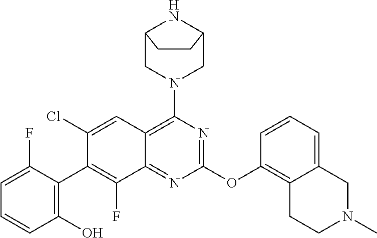 [SMILES: CN1CCc2c(cccc2Oc2nc(N3CC4CCC(C3)N4)c3cc(Cl)c(-c4c(O)cccc4F)c(F)c3n2)C1] | 2-(4-((1R,5S)-3,8- diazabicyclo[3.2.1]octan-3-yl)-6- chloro-8-fluoro-2-((2-methyl- 1,2,3,4-tetrahydroisoquinolin-5- yl)oxy)quinazolin-7-yl)-3- fluorophenol | 564.2 |
| P-0334 | 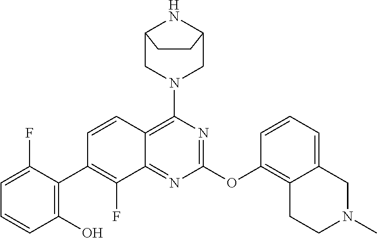 [SMILES: CN1CCc2c(cccc2Oc2nc(N3CC4CCC(C3)N4)c3ccc(-c4c(O)cccc4F)c(F)c3n2)C1] | 2-(4-((1R,5S)-3,8- diazabicyclo[3.2.1]octan-3-yl)-8- fluoro-2-((2-methyl-1,2,3,4- tetrahydroisoquinolin-5- yl)oxy)quinazolin-7-yl)-3- fluorophenol | 530.1 |
| P-0335 | 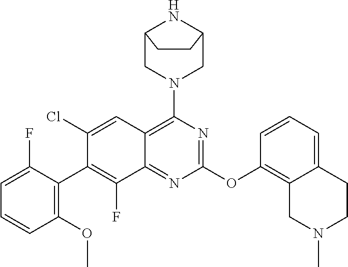 [SMILES: COc1cccc(F)c1-c1c(Cl)cc2c(N3CC4CCC(C3)N4)nc(Oc3cccc4c3CN(C)CC4)nc2c1F] | 4-((1R,5S)-3,8- diazabicyclo[3.2.1]octan-3-yl)-6- chloro-8-fluoro-7-(2-fluoro-6- methoxyphenyl)-2-((2-methyl- 1,2,3,4-tetrahydroisoquinolin-8- yl)oxy)quinazoline | 578.1 |
| P-0336 | 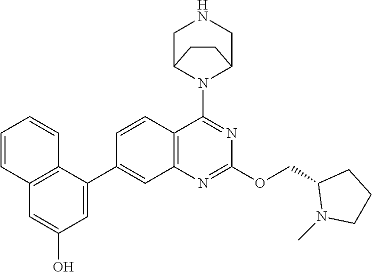 [SMILES: CN1CCC[C@H]1COc1nc(N2C3CCC2CNC3)c2ccc(-c3cc(O)cc4ccccc34)cc2n1] | 4-(4-((1R,5S)-3,8- diazabicyclo[3.2.1]octan-8-yl)-2- (((S)-1-methylpyrrolidin-2- yl)methoxy)quinazolin-7- yl)naphthalen-2-ol | 496.3 |
| P-0337 | 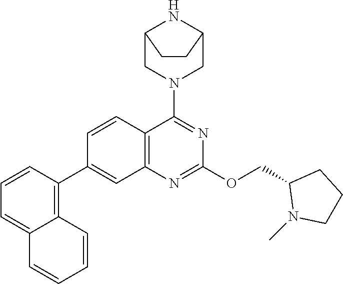 [SMILES: CN1CCC[C@H]1COc1nc(N2CC3CCC(C2)N3)c2ccc(-c3cccc4ccccc34)cc2n1] | 4-((1R,5S)-3,8- diazabicyclo[3.2.1]octan-3-yl)-2- (((S)-1-methylpyrrolidin-2- yl)methoxy)-7-(naphthalen-1- yl)quinazoline | 480.4 |
| P-0338 | 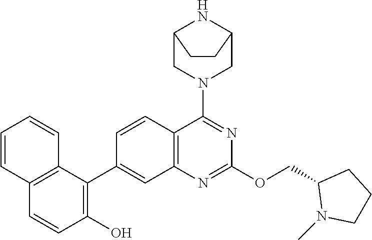 [SMILES: CN1CCC[C@H]1COc1nc(N2CC3CCC(C2)N3)c2ccc(-c3c(O)ccc4ccccc34)cc2n1] | 1-(4-((1R,5S)-3,8- diazabicyclo[3.2.1]octan-3-yl)-2- (((S)-1-methylpyrrolidin-2- yl)methoxy)quinazolin-7- yl)naphthalen-2-ol | 496.4 |
| P-0339 | 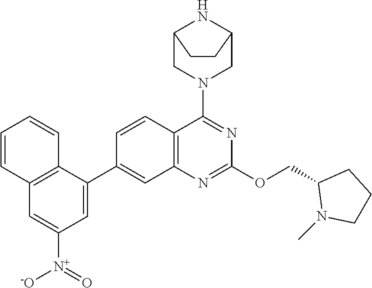 | 4-((1R,5S)-3,8- diazabicyclo[3.2.1]octan-3-yl)-2- (((S)-1-methylpyrrolidin-2- yl)methoxy)-7-(3-nitronaphthalen- 1-yl)quinazoline | 525.3 |
| P-0340 | 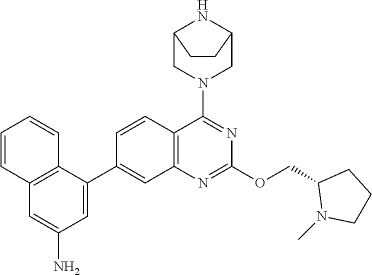 [SMILES: CN1CCC[C@H]1COc1nc(N2CC3CCC(C2)N3)c2ccc(-c3cc(N)cc4ccccc34)cc2n1] | 4-(4-((1R,5S)-3,8- diazabicyclo[3.2.1]octan-3-yl)-2- (((S)-1-methylpyrrolidin-2- yl)methoxy)quinazolin-7- yl)naphthalen-2-amine | 495.3 |
| P-0341 | 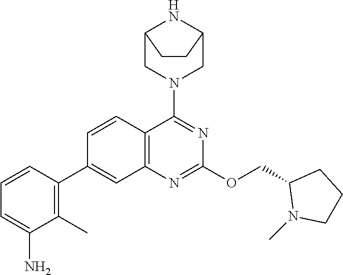 [SMILES: Cc1c(N)cccc1-c1ccc2c(N3CC4CCC(C3)N4)nc(OC[C@@H]3CCCN3C)nc2c1] | 3-(4-((1R,5S)-3,8- diazabicyclo[3.2.1]octan-3-yl)-2- (((S)-1-methylpyrrolidin-2- yl)methoxy)quinazolin-7-yl)-2- methylaniline | 459.4 |
| P-0342 | 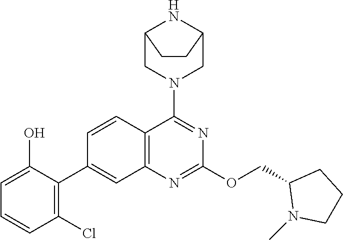 [SMILES: CN1CCC[C@H]1COc1nc(N2CC3CCC(C2)N3)c2ccc(-c3c(O)cccc3Cl)cc2n1] | 2-(4-((1R,5S)-3,8- diazabicyclo[3.2.1]octan-3-yl)-2- (((S)-1-methylpyrrolidin-2- yl)methoxy)quinazolin-7-yl)-3- chlorophenol | 480.3 |
| P-0343 | 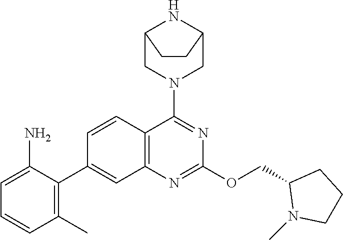 [SMILES: Cc1cccc(N)c1-c1ccc2c(N3CC4CCC(C3)N4)nc(OC[C@@H]3CCCN3C)nc2c1] | 2-(4-((1R,5S)-3,8- diazabicyclo[3.2.1]octan-3-yl)-2- (((S)-1-methylpyrrolidin-2- yl)methoxy)quinazolin-7-yl)-3- methylaniline | 459.4 |
| P-0344 | 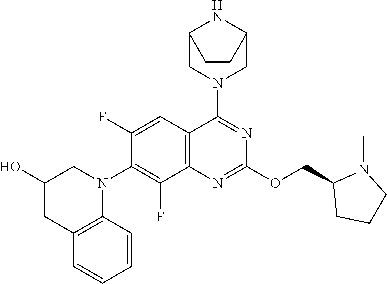 [SMILES: CN1CCC[C@H]1COc1nc(N2CC3CCC(C2)N3)c2cc(F)c(N3CC(O)Cc4ccccc43)c(F)c2n1] | 1-(4-((1R,5S)-3,8- diazabicyclo[3.2.1]octan-3-yl)-6,8- difluoro-2-(((S)-1- methylpyrrolidin-2- yl)methoxy)quinazolin-7-yl)- 1,2,3,4-tetrahydroquinolin-3-ol | 537.4 |
| P-0345 | 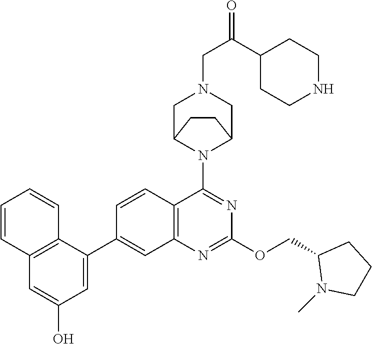 [SMILES: CN1CCC[C@H]1COc1nc(N2C3CCC2CN(CC(=O)C2CCNCC2)C3)c2ccc(-c3cc(O)cc4ccccc34)cc2n1] | 2-((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)-1- (piperidin-4-yl)ethan-1-one | 621.7 |
| P-0346 | 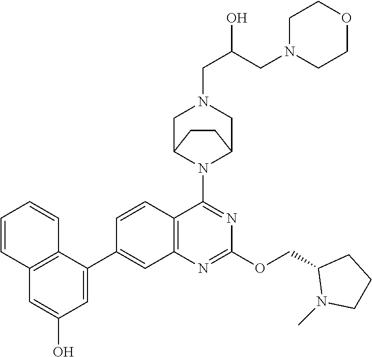 [SMILES: CN1CCC[C@H]1COc1nc(N2C3CCC2CN(CC(O)CN2CCOCC2)C3)c2ccc(-c3cc(O)cc4ccccc34)cc2n1] | 4-(4-((1R,5S)-3-(2-hydroxy-3- morpholinopropyl)-3,8- diazabicyclo[3.2.1]octan-8-yl)-2- (((S)-1-methylpyrrolidin-2- yl)methoxy)quinazolin-7- yl)naphthalen-2-ol | 639.4 |
| P-0347 | 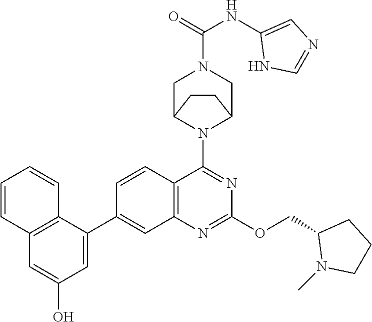 [SMILES: CN1CCC[C@H]1COc1nc(N2C3CCC2CN(C(=O)Nc2cnc[nH]2)C3)c2ccc(-c3cc(O)cc4ccccc34)cc2n1] | (1R,5S)-8-(7-(3- hydroxynaphthalen-1-yl)-2-(((S)-1- methylpyrrolidin-2- yl)methoxy)quinazolin-4-yl)-N- (1H-imidazol-5-yl)-3,8- diazabicyclo[3.2.1]octane-3- carboxamide | 605.8 |
| P-0348 | 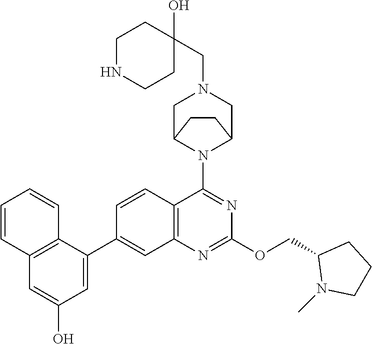 [SMILES: CN1CCC[C@H]1COc1nc(N2C3CCC2CN(CC2(O)CCNCC2)C3)c2ccc(-c3cc(O)cc4ccccc34)cc2n1] | 4-(((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3- yl)methyl)piperidin-4-ol | 609.5 |
| P-0349 | 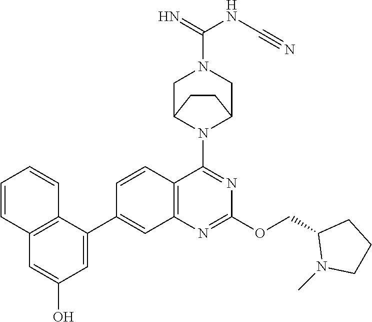 [SMILES: CN1CCC[C@H]1COc1nc(N2C3CCC2CN(C(=N)NC#N)C3)c2ccc(-c3cc(O)cc4ccccc34)cc2n1] | (1R,5S)-N-cyano-8-(7-(3- hydroxynaphthalen-1-yl)-2-(((S)-1- methylpyrrolidin-2- yl)methoxy)quinazolin-4-yl)-3,8- diazabicyclo[3.2.1]octane-3- carboximidamide | 563.5 |
| P-0350 | 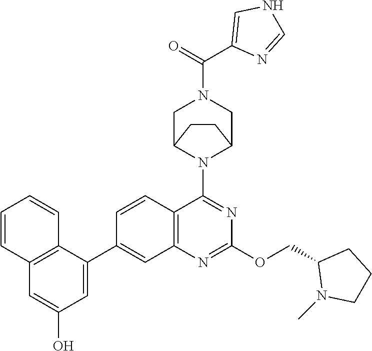 [SMILES: CN1CCC[C@H]1COc1nc(N2C3CCC2CN(C(=O)c2c[nH]cn2)C3)c2ccc(-c3cc(O)cc4ccccc34)cc2n1] | ((1R,5S)-8-(7-(3- hydroxynaphthalen-1-yl)-2-(((S)-1- methylpyrrolidin-2- yl)methoxy)quinazolin-4-yl)-3,8- diazabicyclo[3.2.1]octan-3-yl)(1H- imidazol-4-yl)methanone | 590.5 |
| P-0351 | 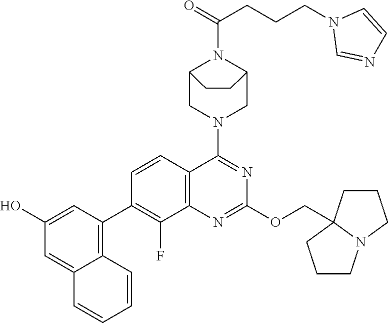 [SMILES: O=C(CCCn1ccnc1)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-4- (1H-imidazol-1-yl)butan-1-one | 676.4 |
| P-0352 | 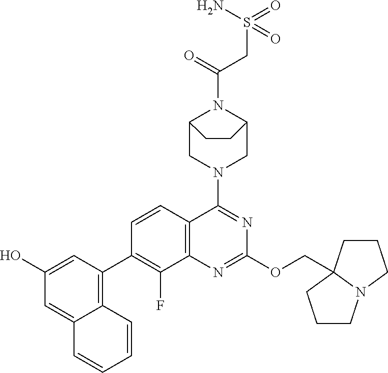 [SMILES: NS(=O)(=O)CC(=O)N1C2CCC1CN(c1nc(OCC34CCCN3CCC4)nc3c(F)c(-c4cc(O)cc5ccccc45)ccc13)C2] | 2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethane-1-sulfonamide | 661.4 |
| P-0353 | 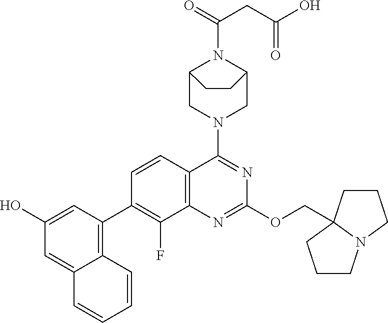 [SMILES: O=C(O)CC(=O)N1C2CCC1CN(c1nc(OCC34CCCN3CCC4)nc3c(F)c(-c4cc(O)cc5ccccc45)ccc13)C2] | 3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanoic acid | 626.4 |
| P-0354 | 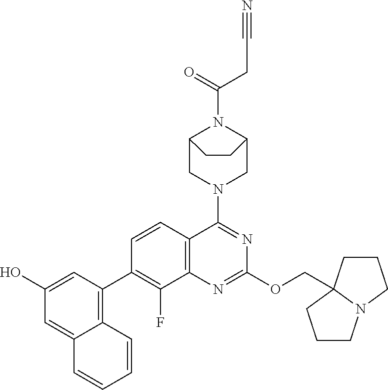 [SMILES: N#CCC(=O)N1C2CCC1CN(c1nc(OCC34CCCN3CCC4)nc3c(F)c(-c4cc(O)cc5ccccc45)ccc13)C2] | 3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanenitrile | 607.4 |
| P-0355 | 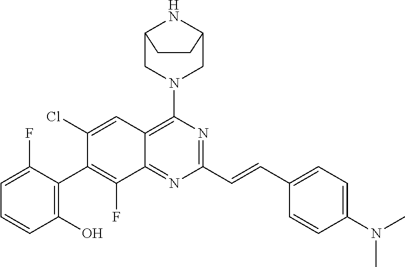 [SMILES: CN(C)c1ccc(/C=C/c2nc(N3CC4CCC(C3)N4)c3cc(Cl)c(-c4c(O)cccc4F)c(F)c3n2)cc1] | 2-(4-((1R,5S)-3,8- diazabicyclo[3.2.1]octan-3-yl)-6- chloro-2-((E)-4- (dimethylamino)styryl)-8- fluoroquinazolin-7-yl)-3- fluorophenol | 548.2 |
| P-0356 | 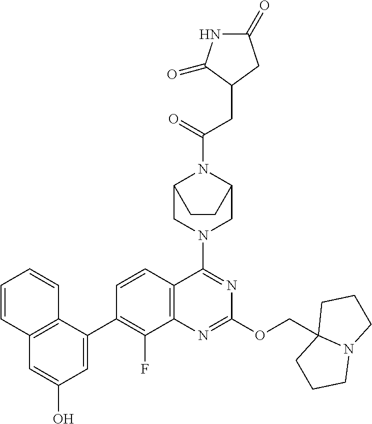 [SMILES: O=C1CC(CC(=O)N2C3CCC2CN(c2nc(OCC45CCCN4CCC5)nc4c(F)c(-c5cc(O)cc6ccccc56)ccc24)C3)C(=O)N1] | 3-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)pyrrolidine-2,5-dione | 679.7 |
| P-0357 | 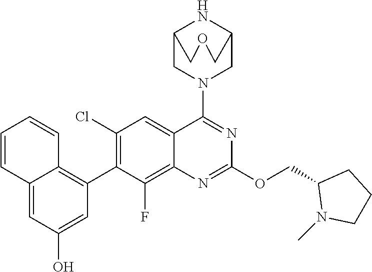 [SMILES: CN1CCC[C@H]1COc1nc(N2CC3COCC(C2)N3)c2cc(Cl)c(-c3cc(O)cc4ccccc34)c(F)c2n1] | 4-(4-(3-oxa-7,9- diazabicyclo[3.3.1]nonan-7-yl)-6- chloro-8-fluoro-2-(((S)-1- methylpyrrolidin-2- yl)methoxy)quinazolin-7- yl)naphthalen-2-ol | 564.2 |
| P-0358 | 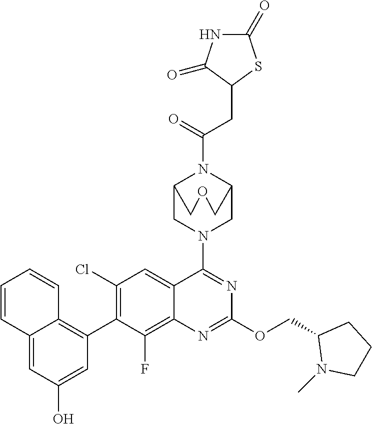 [SMILES: CN1CCC[C@H]1COc1nc(N2CC3COCC(C2)N3C(=O)CC2SC(=O)NC2=O)c2cc(Cl)c(-c3cc(O)cc4ccccc34)c(F)c2n1] | 5-(2-(7-(6-chloro-8-fluoro-7-(3- hydroxynaphthalen-1-yl)-2-(((S)-1- methylpyrrolidin-2- yl)methoxy)quinazolin-4-yl)-3-oxa- 7,9-diazabicyclo[3.3.1]nonan-9-yl)- 2-oxoethyl)thiazolidine-2,4-dione | 721.1 |
| P-0360 | 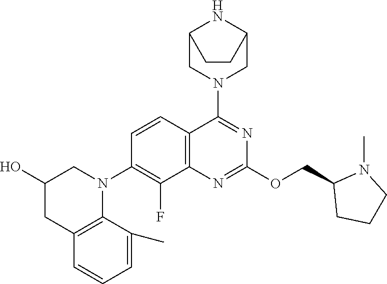 [SMILES: Cc1cccc2c1N(c1ccc3c(N4CC5CCC(C4)N5)nc(OC[C@@H]4CCCN4C)nc3c1F)CC(O)C2] | 1-(4-((1R,5S)-3,8- diazabicyclo[3.2.1]octan-3-yl)-8- fluoro-2-(((S)-1-methylpyrrolidin- 2-yl)methoxy)quinazolin-7-yl)-8- methyl-1,2,3,4-tetrahydroquinolin- 3-ol | 533.3 |
| P-0361 | 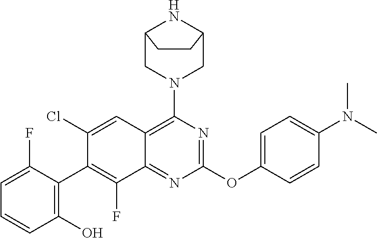 [SMILES: CN(C)c1ccc(Oc2nc(N3CC4CCC(C3)N4)c3cc(Cl)c(-c4c(O)cccc4F)c(F)c3n2)cc1] | 2-(4-((1R,5S)-3,8- diazabicyclo[3.2.1]octan-3-yl)-6- chloro-2-(4- (dimethylamino)phenoxy)-8- fluoroquinazolin-7-yl)-3- fluorophenol | 538.1 |
| P-0362 | 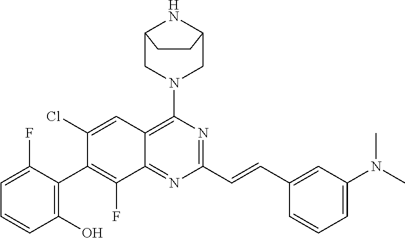 [SMILES: CN(C)c1cccc(/C=C/c2nc(N3CC4CCC(C3)N4)c3cc(Cl)c(-c4c(O)cccc4F)c(F)c3n2)c1] | 2-(4-((1R,5S)-3,8- diazabicyclo[3.2.1]octan-3-yl)-6- chloro-2-((E)-3- (dimethylamino)styryl)-8- fluoroquinazolin-7-yl)-3- fluorophenol | 548.3 |
| P-0363 | 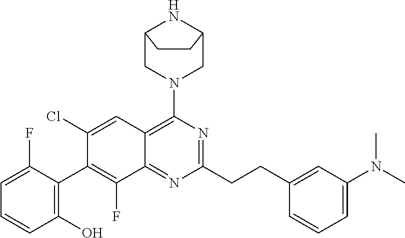 [SMILES: CN(C)c1cccc(CCc2nc(N3CC4CCC(C3)N4)c3cc(Cl)c(-c4c(O)cccc4F)c(F)c3n2)c1] | 2-(4-((1R,5S)-3,8- diazabicyclo[3.2.1]octan-3-yl)-6- chloro-2-(3- (dimethylamino)phenethyl)-8- fluoroquinazolin-7-yl)-3- fluorophenol | 550.1 |
| P-0364 | 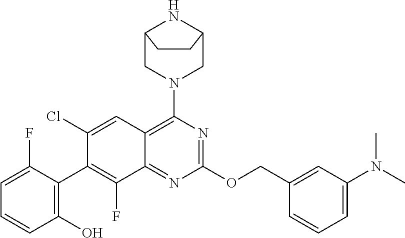 [SMILES: CN(C)c1cccc(COc2nc(N3CC4CCC(C3)N4)c3cc(Cl)c(-c4c(O)cccc4F)c(F)c3n2)c1] | 2-(4-((1R,5S)-3,8- diazabicyclo[3.2.1]octan-3-yl)-6- chloro-2-((3- (dimethylamino)benzyl)oxy)-8- fluoroquinazolin-7-yl)-3- fluorophenol | 552.1 |
| P-0365 | 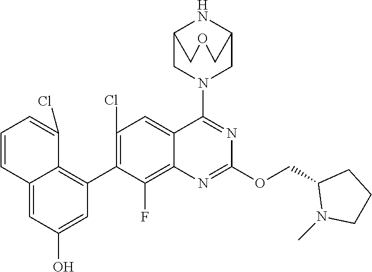 [SMILES: CN1CCC[C@H]1COc1nc(N2CC3COCC(C2)N3)c2cc(Cl)c(-c3cc(O)cc4cccc(Cl)c34)c(F)c2n1] | 4-(4-(3-oxa-7,9- diazabicyclo[3.3.1]nonan-7-yl)-6- chloro-8-fluoro-2-(((S)-1- methylpyrrolidin-2- yl)methoxy)quinazolin-7-yl)-5- chloronaphthalen-2-ol | 598.1 |
| P-0366 | 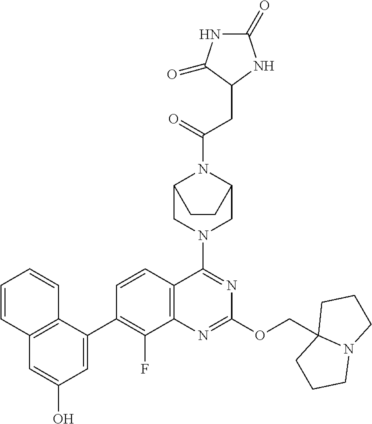 [SMILES: O=C1NC(=O)C(CC(=O)N2C3CCC2CN(c2nc(OCC45CCCN4CCC5)nc4c(F)c(-c5cc(O)cc6ccccc56)ccc24)C3)N1] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethylimidazolidine-2,4-dione | 680.2 |
| P-0368 | 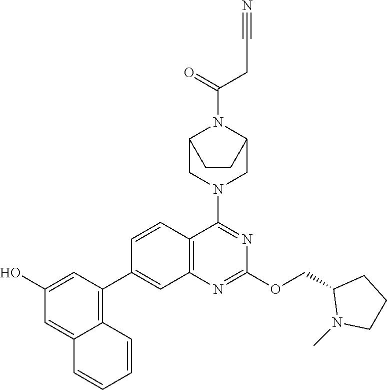 [SMILES: CN1CCC[C@H]1COc1nc(N2CC3CCC(C2)N3C(=O)CC#N)c2ccc(-c3cc(O)cc4ccccc34)cc2n1] | 3-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- oxopropanenitrile | 563.3 |
| P-0369 | 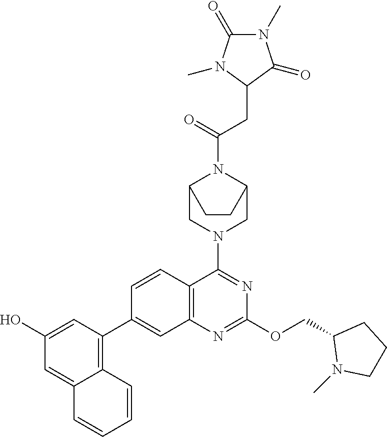 [SMILES: CN1C(=O)C(CC(=O)N2C3CCC2CN(c2nc(OC[C@@H]4CCCN4C)nc4cc(-c5cc(O)cc6ccccc56)ccc24)C3)N(C)C1=O] | 5-(2-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethyl)-1,3- dimethylimidazolidine-2,4-dione | 664.4 |
| P-0370 | 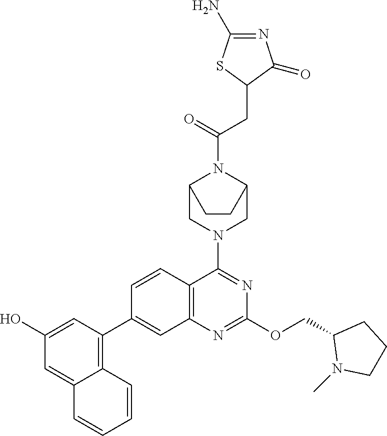 [SMILES: CN1CCC[C@H]1COc1nc(N2CC3CCC(C2)N3C(=O)CC2SC(N)=NC2=O)c2ccc(-c3cc(O)cc4ccccc34)cc2n1] | 2-amino-5-(2-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethyl)thiazol-4(5H)-one | 652.3 |
| P-0371 | 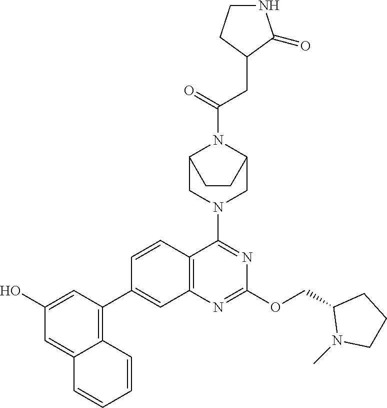 [SMILES: CN1CCC[C@H]1COc1nc(N2CC3CCC(C2)N3C(=O)CC2CCNC2=O)c2ccc(-c3cc(O)cc4ccccc34)cc2n1] | 3-(2-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethyl)pyrrolidin-2-one | 621.4 |
| P-0372 | 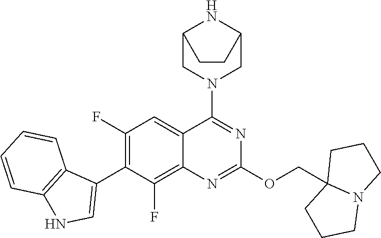 [SMILES: Fc1cc2c(N3CC4CCC(C3)N4)nc(OCC34CCCN3CCC4)nc2c(F)c1-c1c[nH]c2ccccc12] | 4-((1R,5S)-3,8- diazabicyclo[3.2.1]octan-3-yl)-6,8- difluoro-7-(1H-indol-3-yl)-2- ((tetrahydro-1H-pyrrolizin-7a(5H)- yl)methoxy)quinazoline | 531.6 |
| P-0373 | 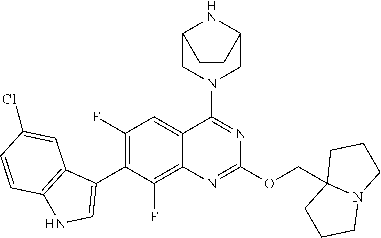 [SMILES: Fc1cc2c(N3CC4CCC(C3)N4)nc(OCC34CCCN3CCC4)nc2c(F)c1-c1c[nH]c2ccc(Cl)cc12] | 4-((1R,5S)-3,8- diazabicyclo[3.2.1]octan-3-yl)-7- (5-chloro-1H-indol-3-yl)-6,8- difluoro-2-((tetrahydro-1H- pyrrolizin-7a(5H)- yl)methoxy)quinazoline | 565.6 |
| P-0374 | 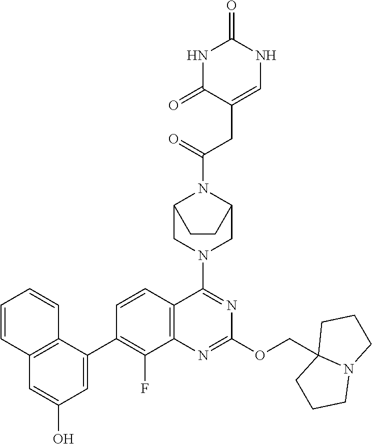 [SMILES: O=C(Cc1c[nH]c(=O)[nH]c1=O)N1C2CCC1CN(c1nc(OCC34CCCN3CCC4)nc3c(F)c(-c4cc(O)cc5ccccc45)ccc13)C2] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)pyrimidine-2,4(1H,3H)- dione | 692.7 |
| P-0375 | 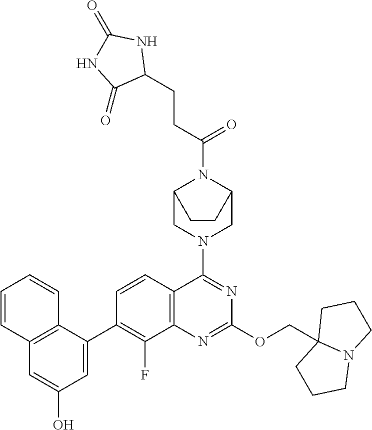 [SMILES: O=C1NC(=O)C(CCC(=O)N2C3CCC2CN(c2nc(OCC45CCCN4CCC5)nc4c(F)c(-c5cc(O)cc6ccccc56)ccc24)C3)N1] | 5-(3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropyl)imidazolidine-2,4-dione | 694.7 |
| P-0376 | 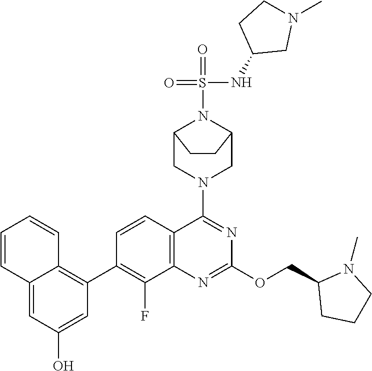 [SMILES: CN1CC[C@@H](NS(=O)(=O)N2C3CCC2CN(c2nc(OC[C@@H]4CCCN4C)nc4c(F)c(-c5cc(O)cc6ccccc56)ccc24)C3)C1] | (1R,5S)-3-(8-fluoro-7-(3- hydroxynaphthalen-1-yl)-2-(((S)-1- methylpyrrolidin-2- yl)methoxy)quinazolin-4-yl)-N- ((R)-1-methylpyrrolidin-3-yl)-3,8- diazabicyclo[3.2.1]octane-8- sulfonamide | 676.4 |
| P-0377 | 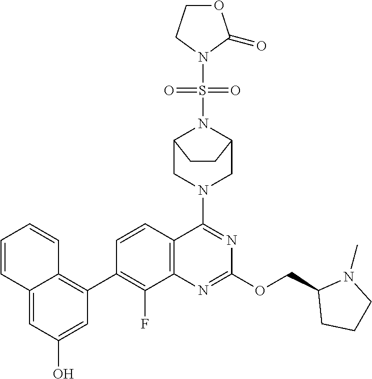 [SMILES: CN1CCC[C@H]1COc1nc(N2CC3CCC(C2)N3S(=O)(=O)N2CCOC2=O)c2ccc(-c3cc(O)cc4ccccc34)c(F)c2n1] | 3-(((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8- yl)sulfonyl)oxazolidin-2-one | 663.3 |
| P-0378 | 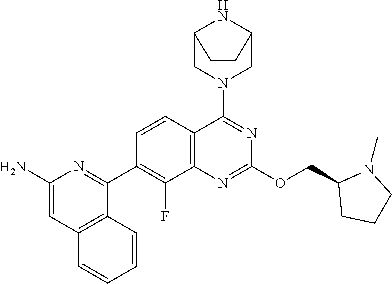 [SMILES: CN1CCC[C@H]1COc1nc(N2CC3CCC(C2)N3)c2ccc(-c3nc(N)cc4ccccc34)c(F)c2n1] | 1-(4-((1R,5S)-3,8- diazabicyclo[3.2.1]octan-3-yl)-8- fluoro-2-(((S)-1-methylpyrrolidin- 2-yl)methoxy)quinazolin-7- yl)isoquinolin-3-amine | 514.4 |
| P-0379 | 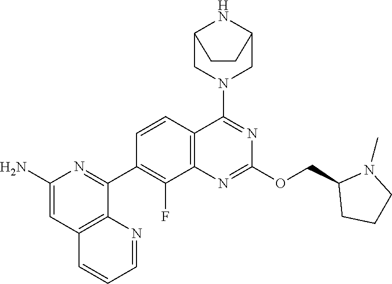 [SMILES: CN1CCC[C@H]1COc1nc(N2CC3CCC(C2)N3)c2ccc(-c3nc(N)cc4cccnc34)c(F)c2n1] | 8-(4-((1R,5S)-3,8- diazabicyclo[3.2.1]octan-3-yl)-8- fluoro-2-(((S)-1-methylpyrrolidin- 2-yl)methoxy)quinazolin-7-yl)-1,7- naphthyridin-6-amine | 515.4 |
| P-0380 | 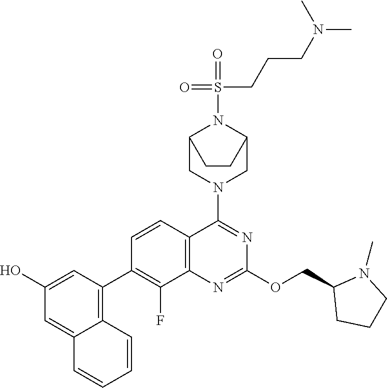 [SMILES: CN(C)CCCS(=O)(=O)N1C2CCC1CN(c1nc(OC[C@@H]3CCCN3C)nc3c(F)c(-c4cc(O)cc5ccccc45)ccc13)C2] | 4-(4-((1R,5S)-8-((3- (dimethylamino)propyl)sulfonyl)- 3,8-diazabicyclo[3.2.1]octan-3-yl)- 8-fluoro-2-(((S)-1- methylpyrrolidin-2- yl)methoxy)quinazolin-7- yl)naphthalen-2-ol | 663.4 |
| P-0381 | 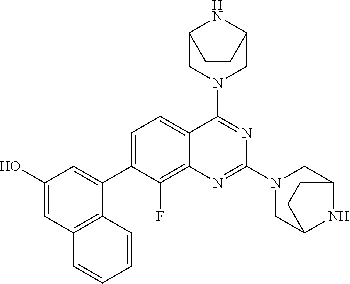 [SMILES: Oc1cc(-c2ccc3c(N4CC5CCC(C4)N5)nc(N4CC5CCC(C4)N5)nc3c2F)c2ccccc2c1] | 4-(4-((1R,5S)-3,8- diazabicyclo[3.2.1]octan-3-yl)-2- (3,8-diazabicyclo[3.2.1]octan-3-yl)- 8-fluoroquinazolin-7- yl)naphthalen-2-ol | 511.6 |
| P-0382 | 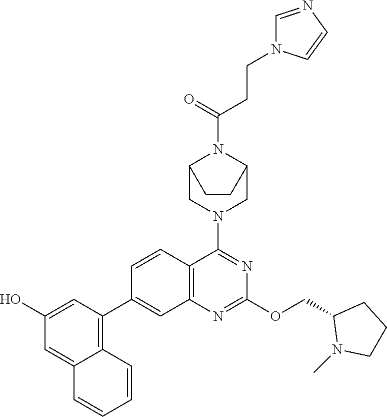 [SMILES: CN1CCC[C@H]1COc1nc(N2CC3CCC(C2)N3C(=O)CCn2ccnc2)c2ccc(-c3cc(O)cc4ccccc34)cc2n1] | 1-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- (1H-imidazol-1-yl)propan-1-one | 618.4 |
| P-0383 | 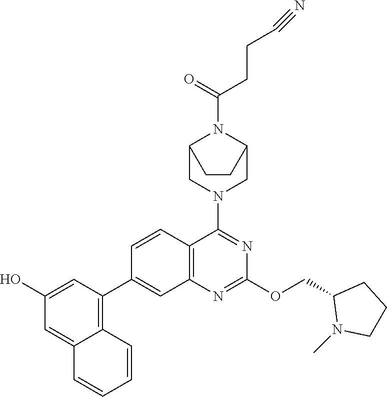 [SMILES: CN1CCC[C@H]1COc1nc(N2CC3CCC(C2)N3C(=O)CCC#N)c2ccc(-c3cc(O)cc4ccccc34)cc2n1] | 4-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-4- oxobutanenitrile | 577.3 |
| P-0384 | 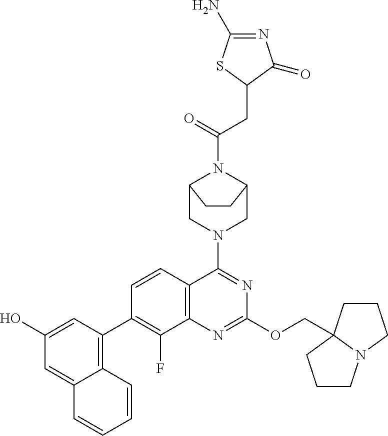 [SMILES: NC1=NC(=O)C(CC(=O)N2C3CCC2CN(c2nc(OCC45CCCN4CCC5)nc4c(F)c(-c5cc(O)cc6ccccc56)ccc24)C3)S1] | 2-amino-5-(2-((1R,5S)-3-(8-fluoro- 7-(3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)thiazol-4 (5H)-one | 696.4 |
| P-0385 | 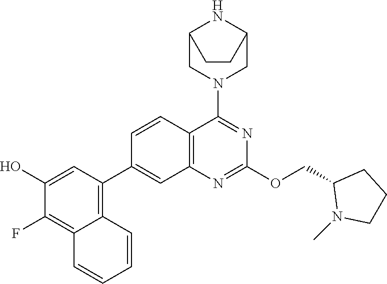 [SMILES: CN1CCC[C@H]1COc1nc(N2CC3CCC(C2)N3)c2ccc(-c3cc(O)c(F)c4ccccc34)cc2n1] | 4-(4-((1R,5S)-3,8- diazabicyclo[3.2.1]octan-3-yl)-2- (((S)-1-methylpyrrolidin-2- yl)methoxy)quinazolin-7-yl)-1- fluoronaphthalen-2-ol | 514.3 |
| P-0386 | 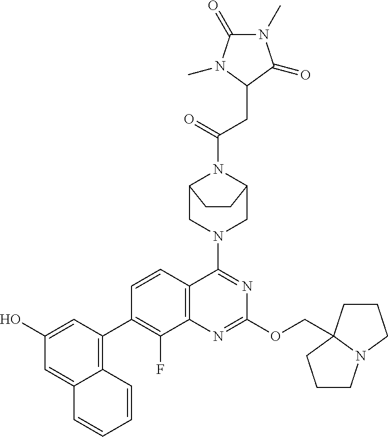 [SMILES: CN1C(=O)C(CC(=O)N2C3CCC2CN(c2nc(OCC45CCCN4CCC5)nc4c(F)c(-c5cc(O)cc6ccccc56)ccc24)C3)N(C)C1=O] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)-1,3- dimethylimidazolidine-2,4-dione | 708.4 |
| P-0387 | 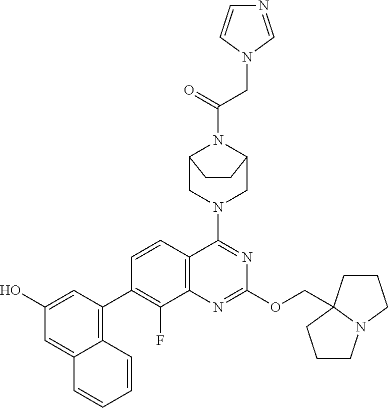 [SMILES: O=C(Cn1ccnc1)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- (1H-imidazol-1-yl)ethan-1-one | 648.4 |
| P-0388 | 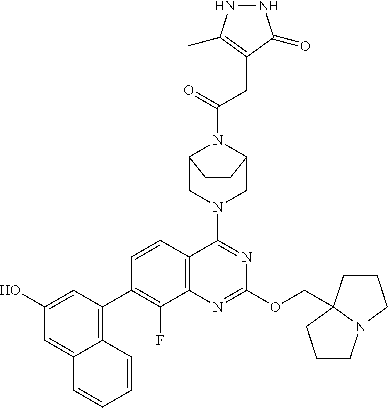 [SMILES: Cc1[nH][nH]c(=O)c1CC(=O)N1C2CCC1CN(c1nc(OCC34CCCN3CCC4)nc3c(F)c(-c4cc(O)cc5ccccc45)ccc13)C2] | 4-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)-5-methyl-1,2-dihydro- 3H-pyrazol-3-one | 678.4 |
| P-0389 | 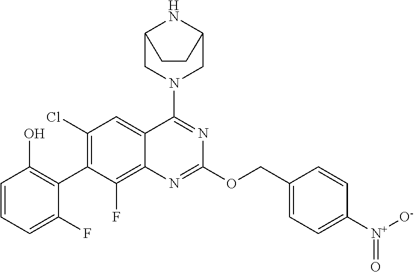 [SMILES: O=[N+]([O-])c1ccc(COc2nc(N3CC4CCC(C3)N4)c3cc(Cl)c(-c4c(O)cccc4F)c(F)c3n2)cc1] | 2-(4-((1R,5S)-3,8- diazabicyclo[3.2.1]octan-3-yl)-6- chloro-8-fluoro-2-((4- nitrobenzyl)oxy)quinazolin-7-yl)-3- fluorophenol | 554.5 |
| P-0390 | 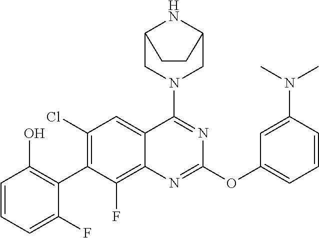 [SMILES: CN(C)c1cccc(Oc2nc(N3CC4CCC(C3)N4)c3cc(Cl)c(-c4c(O)cccc4F)c(F)c3n2)c1] | 2-(4-((1R,5S)-3,8- diazabicyclo[3.2.1]octan-3-yl)-6- chloro-2-(3- (dimethylamino)phenoxy)-8- fluoroquinazolin-7-yl)-3- fluorophenol | 538.1 |
| P-0391 | 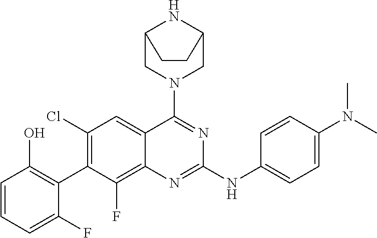 [SMILES: CN(C)c1ccc(Nc2nc(N3CC4CCC(C3)N4)c3cc(Cl)c(-c4c(O)cccc4F)c(F)c3n2)cc1] | 2-(4-((1R,5S)-3,8- diazabicyclo[3.2.1]octan-3-yl)-6- chloro-2-((4- (dimethylamino)phenyl)amino)-8- fluoroquinazolin-7-yl)-3- fluorophenol | 537.1 |
| P-0392 | 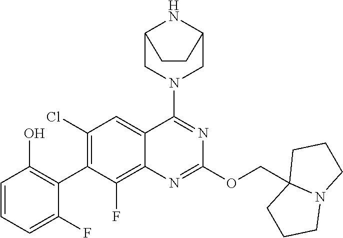 [SMILES: Oc1cccc(F)c1-c1c(Cl)cc2c(N3CC4CCC(C3)N4)nc(OCC34CCCN3CCC4)nc2c1F] | 2-(4-((1R,5S)-3,8- diazabicyclo[3.2.1]octan-3-yl)-6- chloro-8-fluoro-2-((tetrahydro-1H- pyrrolizin-7a(5H)- yl)methoxy)quinazolin-7-yl)-3- fluorophenol | 542.1 |
| P-0393 | 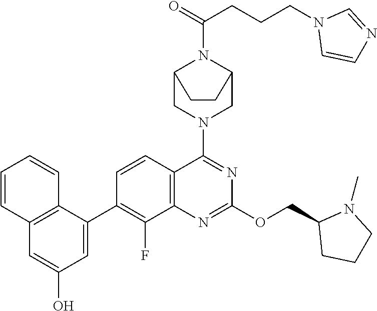 [SMILES: CN1CCC[C@H]1COc1nc(N2CC3CCC(C2)N3C(=O)CCCn2ccnc2)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-4- (1H-imidazol-1-yl)butan-1-one | 650.5 |
| P-0394 | 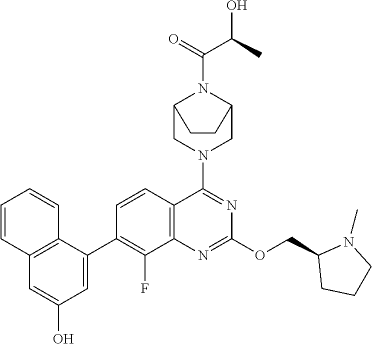 [SMILES: C[C@H](O)C(=O)N1C2CCC1CN(c1nc(OC[C@@H]3CCCN3C)nc3c(F)c(-c4cc(O)cc5ccccc45)ccc13)C2] | (S)-1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- hydroxypropan-1-one | 586.3 |
| P-0395 | 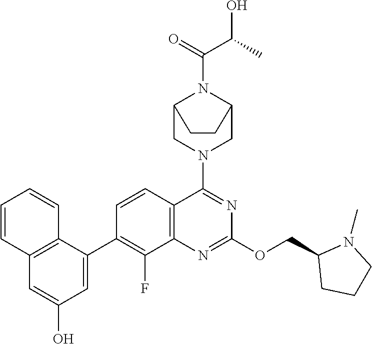 [SMILES: C[C@@H](O)C(=O)N1C2CCC1CN(c1nc(OC[C@@H]3CCCN3C)nc3c(F)c(-c4cc(O)cc5ccccc45)ccc13)C2] | (R)-1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- hydroxypropan-1-one | 586.3 |
| P-0396 | 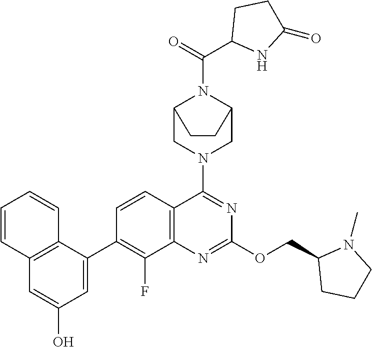 [SMILES: CN1CCC[C@H]1COc1nc(N2CC3CCC(C2)N3C(=O)C2CCC(=O)N2)c2ccc(-c3cc(O)cc4ccccc34)c(F)c2n1] | 5-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- carbonyl)pyrrolidin-2-one | 625.6 |
| P-0397 | 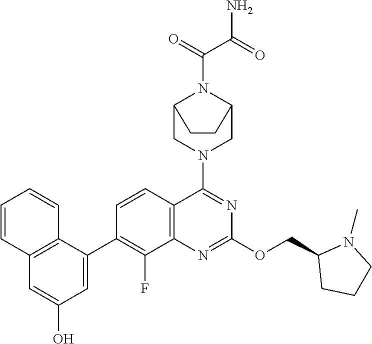 [SMILES: CN1CCC[C@H]1COc1nc(N2CC3CCC(C2)N3C(=O)C(N)=O)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoacetamide | 585.4 |
| P-0398 | 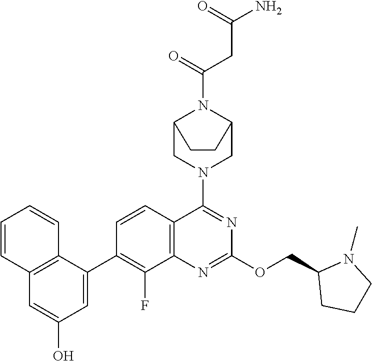 [SMILES: CN1CCC[C@H]1COc1nc(N2CC3CCC(C2)N3C(=O)CC(N)=O)c2ccc(-c3cc(O)cc4ccccc34)c(F)c2n1] | 3-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- oxopropanamide | 599.5 |
| P-0399 | 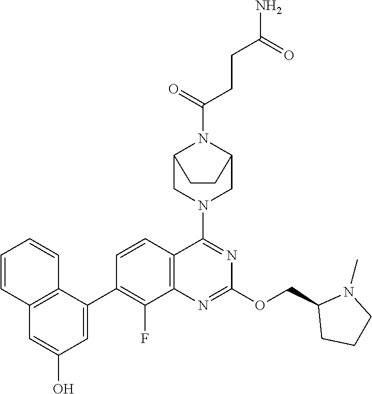 [SMILES: CN1CCC[C@H]1COc1nc(N2CC3CCC(C2)N3C(=O)CCC(N)=O)c2ccc(-c3cc(O)cc4ccccc34)c(F)c2n1] | 4-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-4- oxobutanamide | 613.3 |
| P-0400 | 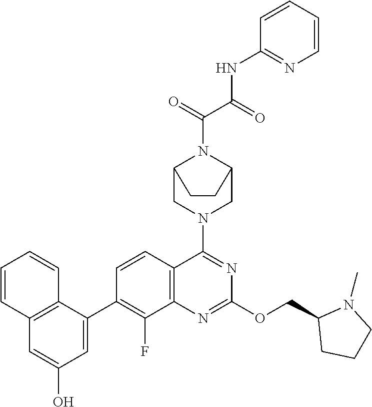 [SMILES: CN1CCC[C@H]1COc1nc(N2CC3CCC(C2)N3C(=O)C(=O)Nc2ccccn2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxo-N-(pyridin-2-yl)acetamide | 662.5 |
| P-0401 | 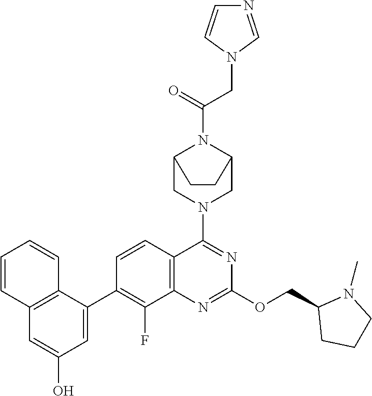 [SMILES: CN1CCC[C@H]1COc1nc(N2CC3CCC(C2)N3C(=O)Cn2ccnc2)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (1H-imidazol-1-yl)ethan-1-one | 622.6 |
| P-0402 | 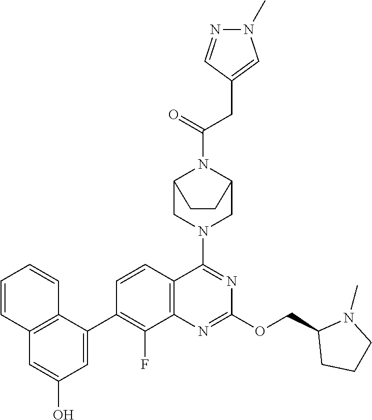 [SMILES: CN1CCC[C@H]1COc1nc(N2CC3CCC(C2)N3C(=O)Cc2cnn(C)c2)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (1-methyl-1H-pyrazol-4-yl)ethan-1- one | 636.4 |
| P-0403 | 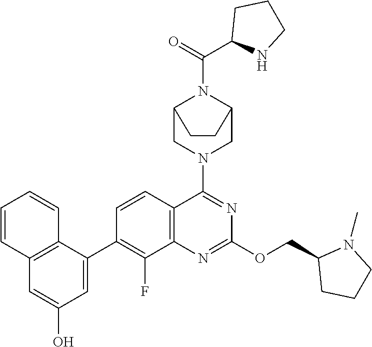 [SMILES: CN1CCC[C@H]1COc1nc(N2CC3CCC(C2)N3C(=O)[C@H]2CCCN2)c2ccc(-c3cc(O)cc4ccccc34)c(F)c2n1] | 4-((1R,5S)-8-(D-prolyl)-3,8- diazabicyclo[3.2.1]octan-3-yl)-8- fluoro-7-(3-hydroxynaphthalen-1- yl)-2-(((S)-1-methylpyrrolidin-2- yl)methoxy)quinazoline | 611.5 |
| P-0404 | 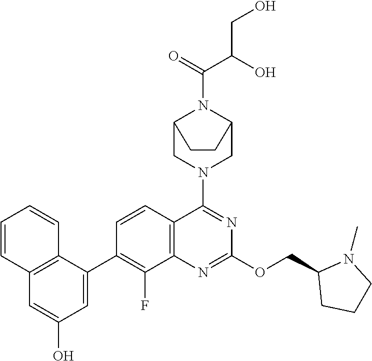 [SMILES: CN1CCC[C@H]1COc1nc(N2CC3CCC(C2)N3C(=O)C(O)CO)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2,3- dihydroxypropan-1-one | 602.8 |
| P-0405 | 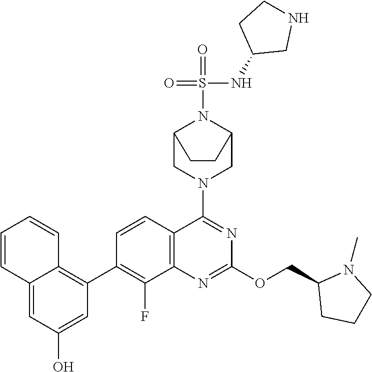 [SMILES: CN1CCC[C@H]1COc1nc(N2CC3CCC(C2)N3S(=O)(=O)N[C@@H]2CCNC2)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-N- ((R)-pyrrolidin-3-yl)-3,8- diazabicyclo[3.2.1]octane-8- sulfonamide | 662.3 |
| P-0406 | 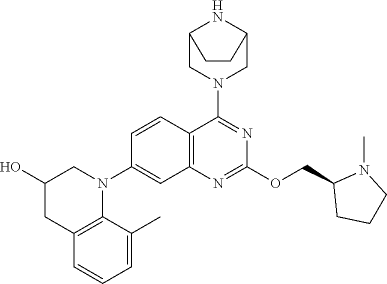 [SMILES: Cc1cccc2c1N(c1ccc3c(N4CC5CCC(C4)N5)nc(OC[C@@H]4CCCN4C)nc3c1)CC(O)C2] | 1-(4-((1R,5S)-3,8- diazabicyclo[3.2.1]octan-3-yl)-2- (((S)-1-methylpyrrolidin-2- yl)methoxy)quinazolin-7-yl)-8- methyl-1,2,3,4-tetrahydroquinolin- 3-ol | 515.4 |
| P-0407 | 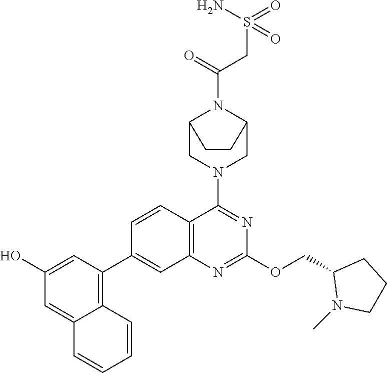 [SMILES: CN1CCC[C@H]1COc1nc(N2CC3CCC(C2)N3C(=O)CS(N)(=O)=O)c2ccc(-c3cc(O)cc4ccccc34)cc2n1] | 2-((1R,5S)-3-(7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethane-1-sulfonamide | 617.3 |
| P-0408 | 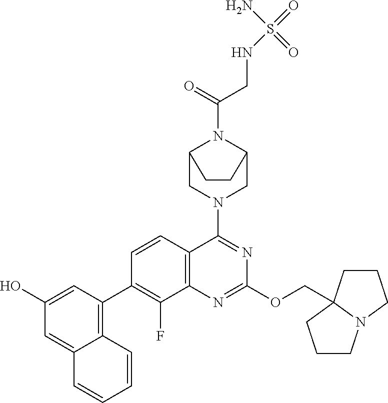 [SMILES: NS(=O)(=O)NCC(=O)N1C2CCC1CN(c1nc(OCC34CCCN3CCC4)nc3c(F)c(-c4cc(O)cc5ccccc45)ccc13)C2] | N-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)aminosulfonamide | 676.3 |
| P-0409 | 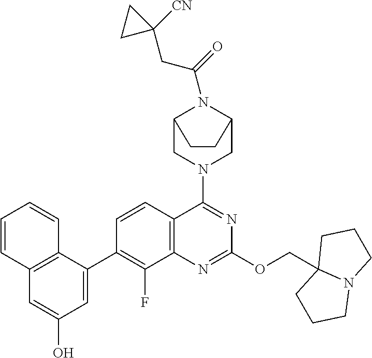 [SMILES: N#CC1(CC(=O)N2C3CCC2CN(c2nc(OCC45CCCN4CCC5)nc4c(F)c(-c5cc(O)cc6ccccc56)ccc24)C3)CC1] | 1-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)cyclopropane-1- carbonitrile | 647.3 |
| P-0410 | 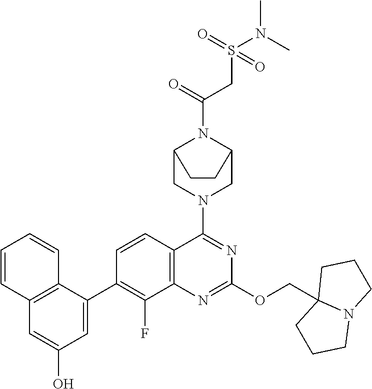 [SMILES: CN(C)S(=O)(=O)CC(=O)N1C2CCC1CN(c1nc(OCC34CCCN3CCC4)nc3c(F)c(-c4cc(O)cc5ccccc45)ccc13)C2] | 2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)- N,N-dimethyl-2-oxoethane-1- sulfonamide | 689.3 |
| P-0411 | 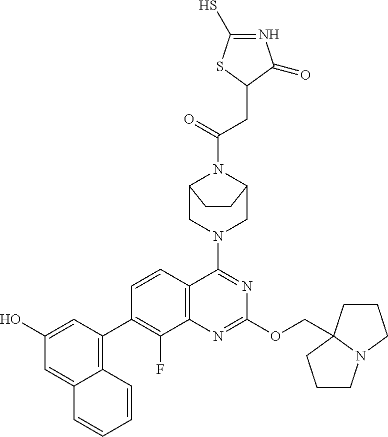 [SMILES: O=C1N=C(S)SC1CC(=O)N1C2CCC1CN(c1nc(OCC34CCCN3CCC4)nc3c(F)c(-c4cc(O)cc5ccccc45)ccc13)C2] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)-2-mercaptothiazol- 4(5H)-one | 713.3 |
| P-0412 | 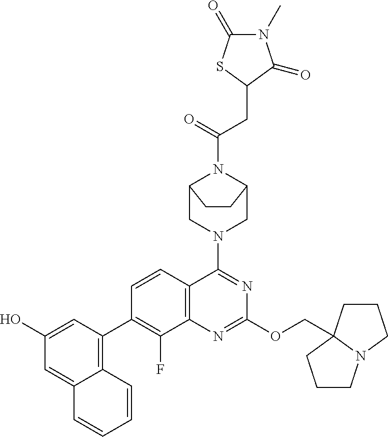 [SMILES: CN1C(=O)SC(CC(=O)N2C3CCC2CN(c2nc(OCC45CCCN4CCC5)nc4c(F)c(-c5cc(O)cc6ccccc56)ccc24)C3)C1=O] | 5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)-3-methylthiazolidine-2,4- dione | 711.4 |
| P-0413 | 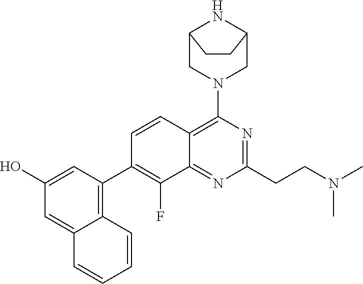 [SMILES: CN(C)CCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2- lamino)ethyl)-8- (2-(dimethylamino)ethyl)-8- fluoroquinazolin-7-yl)naphthalen- 2-ol | 473.2 |
| P-0414 | 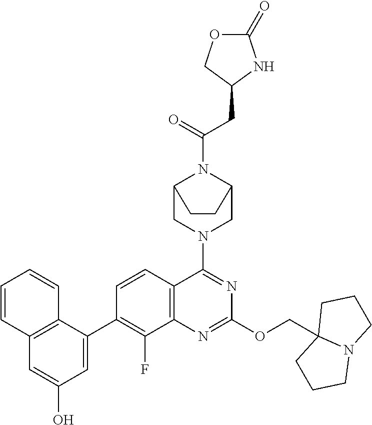 [SMILES: O=C1N[C@@H](CC(=O)N2C3CCC2CN(c2nc(OCC45CCCN4CCC5)nc4c(F)c(-c5cc(O)cc6ccccc56)ccc24)C3)CO1] | (S)-4-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)oxazolidin-2-one | 667.7 |
| P-0415 | 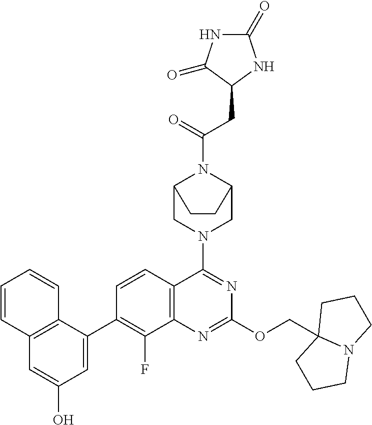 [SMILES: O=C1NC(=O)[C@H](CC(=O)N2C3CCC2CN(c2nc(OCC45CCCN4CCC5)nc4c(F)c(-c5cc(O)cc6ccccc56)ccc24)C3)N1] | (S)-5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethyl)imidazolidine-2,4-dione | 680.7 |
| P-0416 | 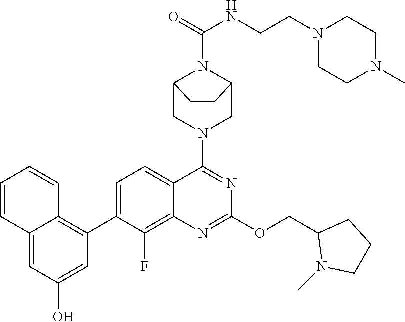 [SMILES: CN1CCN(CCNC(=O)N2C3CCC2CN(c2nc(OCC4CCCN4C)nc4c(F)c(-c5cc(O)cc6ccccc56)ccc24)C3)CC1] | (1R,5S)-3-(8-fluoro-7-(3- hydroxynaphthalen-1-yl)-2-((1- methylpyrrolidin-2- yl)methoxy)quinazolin-4-yl)-N-(2- (4-methylpiperazin-1-yl)ethyl)-3,8- diazabicyclo[3.2.1]octane-8- carboxamide | 683.8 |
| P-0417 | 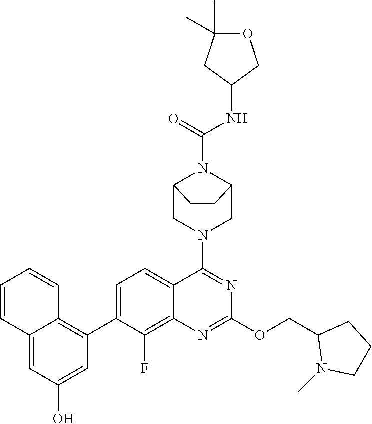 [SMILES: CN1CCCC1COc1nc(N2CC3CCC(C2)N3C(=O)NC2COC(C)(C)C2)c2ccc(-c3cc(O)cc4ccccc34)c(F)c2n1] | (1R,5S)-N-(5,5- dimethyltetrahydrofuran-3-yl)-3-(8- fluoro-7-(3-hydroxynaphthalen-1- yl)-2-((1-methylpyrrolidin-2- yl)methoxy)quinazolin-4-yl)-3,8- diazabicyclo[3.2.1]octane-8- carboxamide | 655.6 |
| P-0418 | 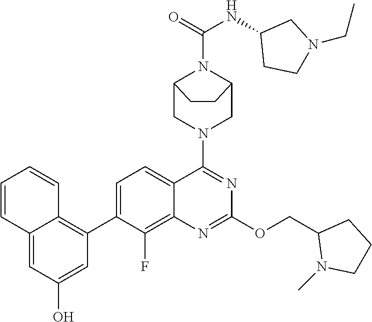 [SMILES: CCN1CC[C@H](NC(=O)N2C3CCC2CN(c2nc(OCC4CCCN4C)nc4c(F)c(-c5cc(O)cc6ccccc56)ccc24)C3)C1] | (1R,5S)-N-((S)-1-ethylpyrrolidin-3- yl)-3-(8-fluoro-7-(3- hydroxynaphthalen-1-yl)-2-((1- methylpyrrolidin-2- yl)methoxy)quinazolin-4-yl)-3,8- diazabicyclo[3.2.1]octane-8- carboxamide | 654.4 |
| P-0419 | 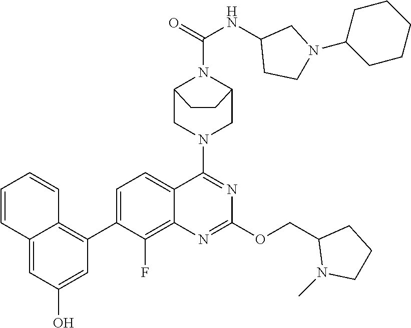 [SMILES: CN1CCCC1COc1nc(N2CC3CCC(C2)N3C(=O)NC2CCN(C3CCCCC3)C2)c2ccc(-c3cc(O)cc4ccccc34)c(F)c2n1] | (1R,5S)-N-(1-cyclohexylpyrrolidin- 3-yl)-3-(8-fluoro-7-(3- hydroxynaphthalen-1-yl)-2-((1- methylpyrrolidin-2- yl)methoxy)quinazolin-4-yl)-3,8- diazabicyclo[3.2.1]octane-8- carboxamide | 708.7 |
| P-0420 | 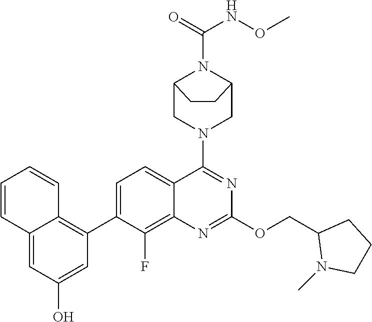 [SMILES: CONC(=O)N1C2CCC1CN(c1nc(OCC3CCCN3C)nc3c(F)c(-c4cc(O)cc5ccccc45)ccc13)C2] | (1R,5S)-3-(8-fluoro-7-(3- hydroxynaphthalen-1-yl)-2-((1- methylpyrrolidin-2- yl)methoxy)quinazolin-4-yl)-N- methoxy-3,8- diazabicyclo[3.2.1]octane-8- carboxamide | 587.2 |
| P-0421 | 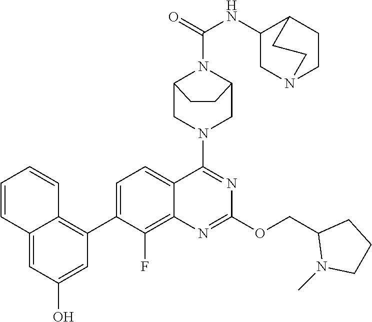 [SMILES: CN1CCCC1COc1nc(N2CC3CCC(C2)N3C(=O)NC2CN3CCC2CC3)c2ccc(-c3cc(O)cc4ccccc34)c(F)c2n1] | (1R,5S)-3-(8-fluoro-7-(3- hydroxynaphthalen-1-yl)-2-((1- methylpyrrolidin-2- yl)methoxy)quinazolin-4-yl)-N- (quinuclidin-3-yl)-3,8- diazabicyclo[3.2.1]octane-8- carboxamide | 666.7 |
| P-0422 | 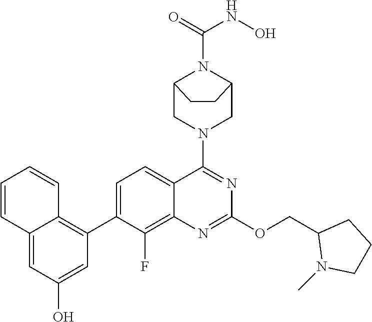 [SMILES: CN1CCCC1COc1nc(N2CC3CCC(C2)N3C(=O)NO)c2ccc(-c3cc(O)cc4ccccc34)c(F)c2n1] | (1R,5S)-3-(8-fluoro-7-(3- hydroxynaphthalen-1-yl)-2-((1- methylpyrrolidin-2- yl)methoxy)quinazolin-4-yl)-N- hydroxy-3,8- diazabicyclo[3.2.1]octane-8- carboxamide | 573.4 |
| P-0423 | 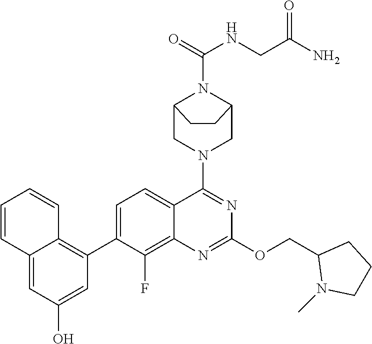 [SMILES: CN1CCCC1COc1nc(N2CC3CCC(C2)N3C(=O)NCC(N)=O)c2ccc(-c3cc(O)cc4ccccc34)c(F)c2n1] | (1R,5S)-N-(2-amino-2-oxoethyl)-3- (8-fluoro-7-(3-hydroxynaphthalen- 1-yl)-2-((1-methylpyrrolidin-2- yl)methoxy)quinazolin-4-yl)-3,8- diazabicyclo[3.2.1]octane-8- carboxamide | 614.5 |
| P-0424 | 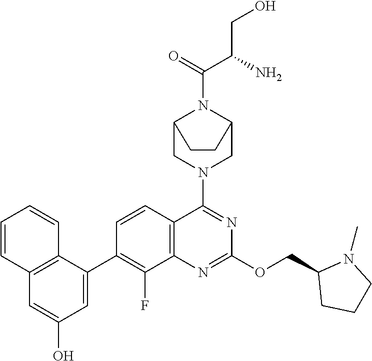 [SMILES: CN1CCC[C@H]1COc1nc(N2CC3CCC(C2)N3C(=O)[C@@H](N)CO)c2ccc(-c3cc(O)cc4ccccc34)c(F)c2n1] | (S)-2-amino-1-((1R,5S)-3-(8- fluoro-7-(3-hydroxynaphthalen-1- yl)-2-(((S)-1-methylpyrrolidin-2- yl)methoxy)quinazolin-4-yl)-3,8- diazabicyclo[3.2.1]octan-8-yl)-3- hydroxypropan-1-one | 601.6 |
| P-0425 | 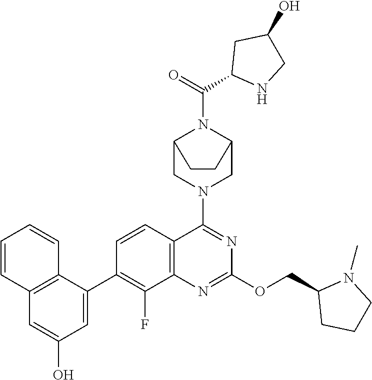 [SMILES: CN1CCC[C@H]1COc1nc(N2CC3CCC(C2)N3C(=O)[C@@H]2C[C@@H](O)CN2)c2ccc(-c3cc(O)cc4ccccc34)c(F)c2n1] | ((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8- yl)((2S,4R)-4-hydroxypyrrolidin-2- yl)methanone | 627.7 |
| P-0426 | 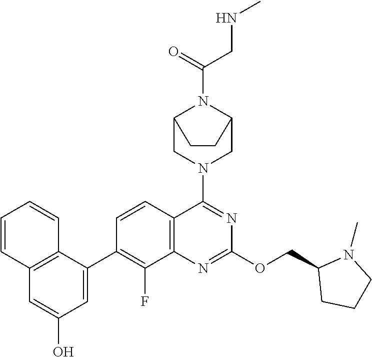 [SMILES: CNCC(=O)N1C2CCC1CN(c1nc(OC[C@@H]3CCCN3C)nc3c(F)c(-c4cc(O)cc5ccccc45)ccc13)C2] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (methylamino)ethan-1-one | 585.4 |
| P-0427 | 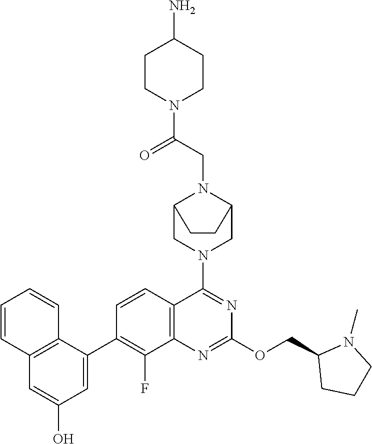 [SMILES: CN1CCC[C@H]1COc1nc(N2CC3CCC(C2)N3CC(=O)N2CCC(N)CC2)c2ccc(-c3cc(O)cc4ccccc34)c(F)c2n1] | 1-(4-aminopiperidin-1-yl)-2- ((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8- yl)ethan-1-one | 654.7 |
| P-0428 | 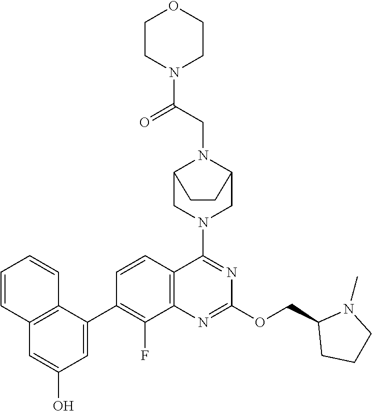 [SMILES: CN1CCC[C@H]1COc1nc(N2CC3CCC(C2)N3CC(=O)N2CCOCC2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-1- morpholinoethan-1-one | 641.5 |
| P-0429 | 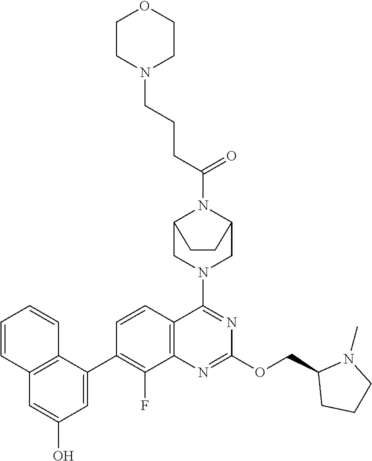 [SMILES: CN1CCC[C@H]1COc1nc(N2CC3CCC(C2)N3C(=O)CCCN2CCOCC2)c2ccc(-c3cc(O)cc4ccccc34)c(F)c2n1] | 1-((1R,5S)-3-(8-fluoro-7-(3- hydroxynaphthalen-1-yl)-2-(((RS)- 1-methylpyrrolidin-2- yl)methoxy)quinazolin-4-yl)-3,8- diazabicyclo[3.2.1]octan-8-yl)-4- morpholinobutan-1-one | 669.7 |
| P-0430 | 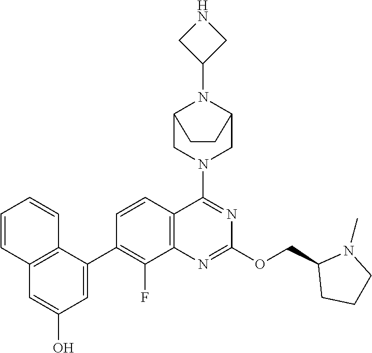 [SMILES: CN1CCC[C@H]1COc1nc(N2CC3CCC(C2)N3C2CNC2)c2ccc(-c3cc(O)cc4ccccc34)c(F)c2n1] | 4-(4-((1R,5S)-8-(azetidin-3-yl)-3,8- diazabicyclo[3.2.1]octan-3-yl)-8- fluoro-2-(((S)-1-methylpyrrolidin- 2-yl)methoxy)quinazolin-7- yl)naphthalen-2-ol | 569.5 |
| P-0431 | 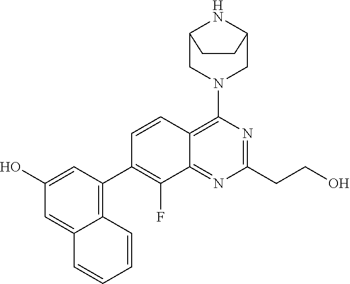 [SMILES: OCCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2- hydroxyethyl)quinazolin-7- yl)naphthalen-2-ol | 445.1 |
| P-0432 | 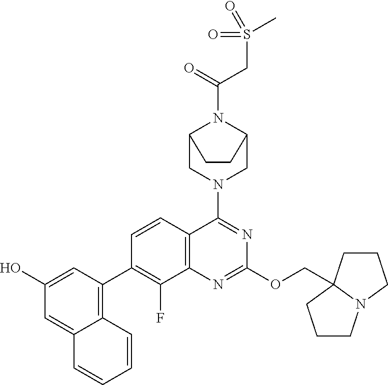 [SMILES: CS(=O)(=O)CC(=O)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- (methylsulfonyl)ethan-1-one | 660.2 |
| P-0433 | 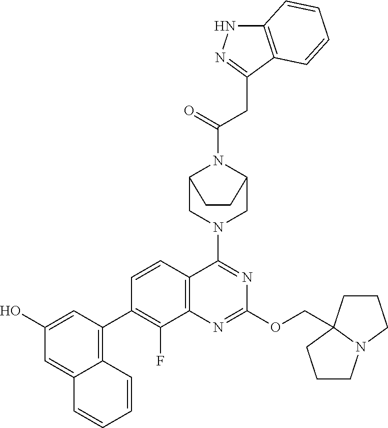 [SMILES: O=C(Cc1n[nH]c2ccccc12)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- (1H-indazol-3-yl)ethan-1-one | 698.3 |
| P-0434 | 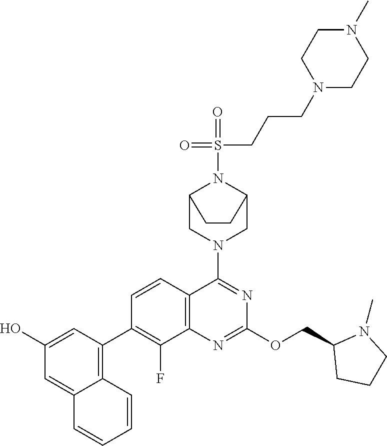 [SMILES: CN1CCN(CCCS(=O)(=O)N2C3CCC2CN(c2nc(OC[C@@H]4CCCN4C)nc4c(F)c(-c5cc(O)cc6ccccc56)ccc24)C3)CC1] | 4-(8-fluoro-4-((1R,5S)-8-((3-(4- methylpiperazin-1- yl)propyl)sulfonyl)-3,8- diazabicyclo[3.2.1]octan-3-yl)-2- (((S)-1-methylpyrrolidin-2- yl)methoxy)quinazolin-7- yl)naphthalen-2-ol | 718.5 |
| P-0435 | 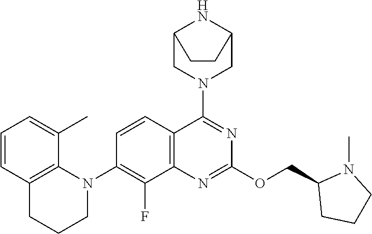 [SMILES: Cc1cccc2c1N(c1ccc3c(N4CC5CCC(C4)N5)nc(OC[C@@H]4CCCN4C)nc3c1F)CCC2] | 4-((1R,5S)-3,8- diazabicyclo[3.2.1]octan-3-yl)-8- fluoro-7-(8-methyl-3,4- dihydroquinolin-1(2H)-yl)-2-(((S)- 1-methylpyrrolidin-2- yl)methoxy)quinazoline | 517.2 |
| P-0436 | 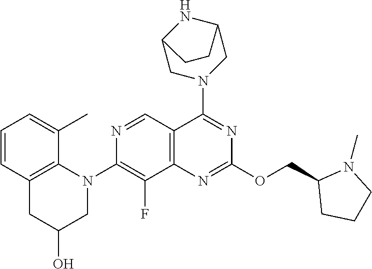 [SMILES: Cc1cccc2c1N(c1ncc3c(N4CC5CCC(C4)N5)nc(OC[C@@H]4CCCN4C)nc3c1F)CC(O)C2] | 1-(4-((1R,5S)-3,8- diazabicyclo[3.2.1]octan-3-yl)-8- fluoro-2-(((S)-1-methylpyrrolidin- 2-yl)methoxy)pyrido[4,3- d]pyrimidin-7-yl)-8-methyl-1,2,3,4- tetrahydroquinolin-3-ol | 534.2 |
| P-0437 | 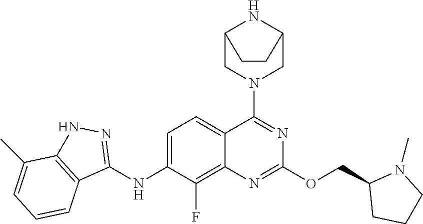 [SMILES: Cc1cccc2c(Nc3ccc4c(N5CC6CCC(C5)N6)nc(OC[C@@H]5CCCN5C)nc4c3F)n[nH]c12] | 4-((1R,5S)-3,8- diazabicyclo[3.2.1]octan-3-yl)-8- fluoro-N-(7-methyl-1H-indazol-3- yl)-2-(((S)-1-methylpyrrolidin-2- yl)methoxy)quinazolin-7-amine | 517.2 |
| P-0438 | 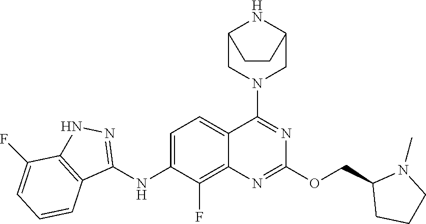 [SMILES: CN1CCC[C@H]1COc1nc(N2CC3CCC(C2)N3)c2ccc(Nc3n[nH]c4c(F)cccc34)c(F)c2n1] | 4-((1R,5S)-3,8- diazabicyclo[3.2.1]octan-3-yl)-8- fluoro-N-(7-fluoro-1H-indazol-3- yl)-2-(((S)-1-methylpyrrolidin-2- yl)methoxy)quinazolin-7-amine | 521.2 |
| P-0439 | 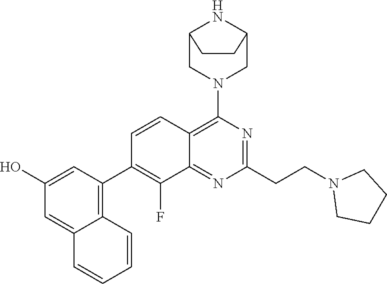 [SMILES: Oc1cc(-c2ccc3c(N4CC5CCC(C4)N5)nc(CCN4CCCC4)nc3c2F)c2ccccc2c1] | 4-(4-((1R,5S)-3,8- diazabicyclo[3.2.1]octan-3-yl)-8- fluoro-2-(2-(pyrrolidin-1- yl)ethyl)quinazolin-7- yl)naphthalen-2-ol | 498.2 |
| P-0440 | 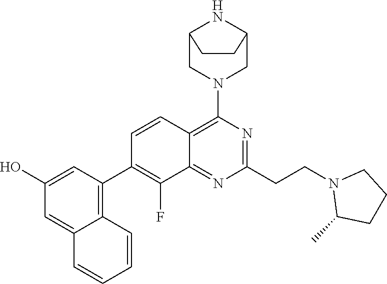 [SMILES: C[C@H]1CCCN1CCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2-((S)-2- methylpyrrolidin-1- yl)ethyl)quinazolin-7- yl)naphthalen-2-ol | 512.2 |
| P-0441 | 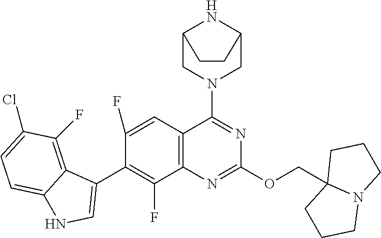 [SMILES: Fc1cc2c(N3CC4CCC(C3)N4)nc(OCC34CCCN3CCC4)nc2c(F)c1-c1c[nH]c2ccc(Cl)c(F)c12] | 4-((1R,5S)-3,8- diazabicyclo[3.2.1]octan-3-yl)-7- (5-chloro-4-fluoro-1H-indol-3-yl)- 6,8-difluoro-2-((tetrahydro-1H- pyrrolizin-7a(5H)- yl)methoxy)quinazoline | 583.6 |
| P-0442 | 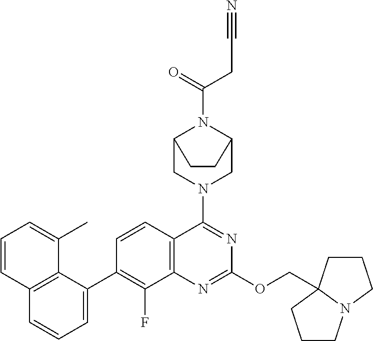 [SMILES: Cc1cccc2cccc(-c3ccc4c(N5CC6CCC(C5)N6C(=O)CC#N)nc(OCC56CCCN5CCC6)nc4c3F)c12] | 3-((1R,5S)-3-(8-fluoro-7-(8- methylnaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanenitrile | 605.4 |
| P-0443 | 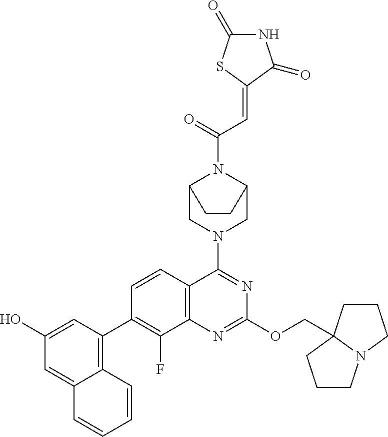 [SMILES: O=C1NC(=O)/C(=C/C(=O)N2C3CCC2CN(c2nc(OCC45CCCN4CCC5)nc4c(F)c(-c5cc(O)cc6ccccc56)ccc24)C3)S1] | (Z)-5-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- oxoethylidene)thiazolidine-2,4- dione | 695.6 |
| P-0444 | 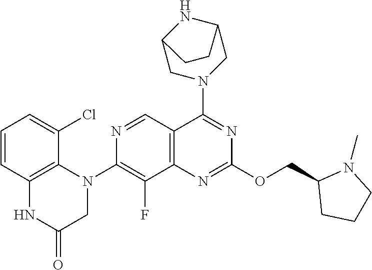 [SMILES: CN1CCC[C@H]1COc1nc(N2CC3CCC(C2)N3)c2cnc(N3CC(=O)Nc4cccc(Cl)c43)c(F)c2n1] | 4-(4-((1R,5S)-3,8- diazabicyclo[3.2.1]octan-3-yl)-8- fluoro-2-(((S)-1-methylpyrrolidin- 2-yl)methoxy)pyrido[4,3- d]pyrimidin-7-yl)-5-chloro-3,4- dihydroquinoxalin-2(1H)-one | 553.2 |
| P-0445 | 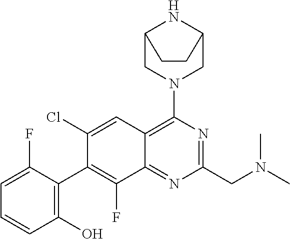 [SMILES: CN(C)Cc1nc(N2CC3CCC(C2)N3)c2cc(Cl)c(-c3c(O)cccc3F)c(F)c2n1] | 2-(4-((1R,5S)-3,8- diazabicyclo[3.2.1]octan-3-yl)-6- chloro-2-((dimethylamino)methyl)- 8-fluoroquinazolin-7-yl)-3- fluorophenol | 460.2 |
| P-0446 | 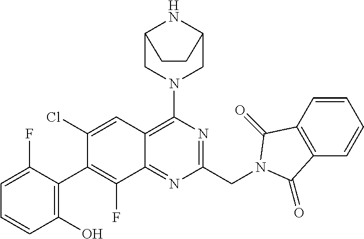 [SMILES: O=C1c2ccccc2C(=O)N1Cc1nc(N2CC3CCC(C2)N3)c2cc(Cl)c(-c3c(O)cccc3F)c(F)c2n1] | 2-((4-((1R,5S)-3,8- diazabicyclo[3.2.1]octan-3-yl)-6- chloro-8-fluoro-7-(2-fluoro-6- hydroxyphenyl)quinazolin-2- yl)methyl)isoindoline-1,3-dione | 562.2 |
| P-0447 | 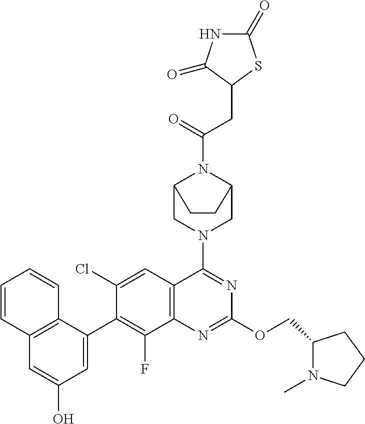 [SMILES: CN1CCC[C@H]1COc1nc(N2CC3CCC(C2)N3C(=O)CC2SC(=O)NC2=O)c2cc(Cl)c(-c3cc(O)cc4ccccc34)c(F)c2n1] | 5-(2-((1R,5S)-3-(6-chloro-8-fluoro- 7-(3-hydroxynaphthalen-1-yl)-2- (((S)-1-methylpyrrolidin-2- yl)methoxy)quinazolin-4-yl)-3,8- diazabicyclo[3.2.1]octan-8-yl)-2- oxoethyl)thiazolidine-2,4-dione | 705.2 |
| P-0448 | 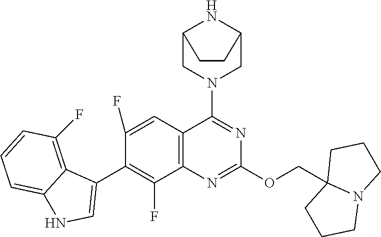 [SMILES: Fc1cc2c(N3CC4CCC(C3)N4)nc(OCC34CCCN3CCC4)nc2c(F)c1-c1c[nH]c2cccc(F)c12] | 4-((1R,5S)-3,8- diazabicyclo[3.2.1]octan-3-yl)-6,8- difluoro-7-(4-fluoro-1H-indol-3- yl)-2-((tetrahydro-1H-pyrrolizin- 7a(5H)-yl)methoxy)quinazoline | 549.2 |
| P-0449 | 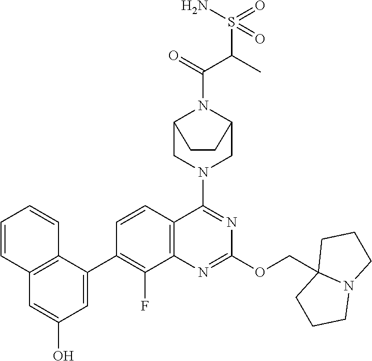 [SMILES: CC(C(=O)N1C2CCC1CN(c1nc(OCC34CCCN3CCC4)nc3c(F)c(-c4cc(O)cc5ccccc45)ccc13)C2)S(N)(=O)=O] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-1- oxopropane-2-sulfonamide | 675.7 |
| P-0450 | 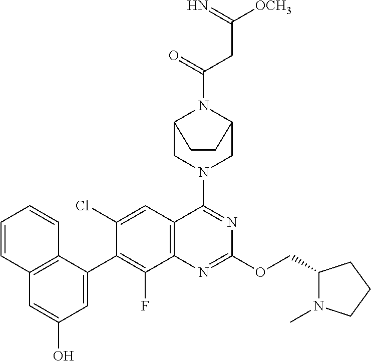 [SMILES: COC(=N)CC(=O)N1C2CCC1CN(c1nc(OC[C@@H]3CCCN3C)nc3c(F)c(-c4cc(O)cc5ccccc45)c(Cl)cc13)C2] | methyl 3-((1R,5S)-3-(6-chloro-8- fluoro-7-(3-hydroxynaphthalen-1- yl)-2-(((S)-1-methylpyrrolidin-2- yl)methoxy)quinazolin-4-yl)-3,8- diazabicyclo[3.2.1]octan-8-yl)-3- oxopropanimidate | 648.6 |
| P-0451 | 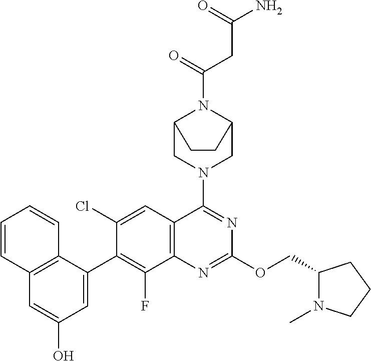 [SMILES: CN1CCC[C@H]1COc1nc(N2CC3CCC(C2)N3C(=O)CC(N)=O)c2cc(Cl)c(-c3cc(O)cc4ccccc34)c(F)c2n1] | 3-((1R,5S)-3-(6-chloro-8-fluoro-7- (3-hydroxynaphthalen-1-yl)-2- (((S)-1-methylpyrrolidin-2- yl)methoxy)quinazolin-4-yl)-3,8- diazabicyclo[3.2.1]octan-8-yl)-3- oxopropanamide | 633.2 |
| P-0452 | 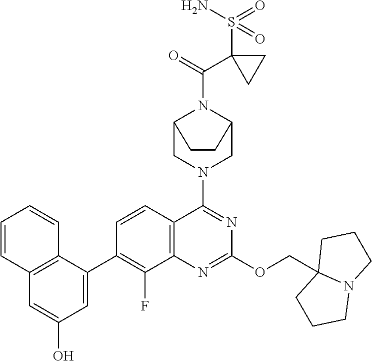 [SMILES: NS(=O)(=O)C1(C(=O)N2C3CCC2CN(c2nc(OCC45CCCN4CCC5)nc4c(F)c(-c5cc(O)cc6ccccc56)ccc24)C3)CC1] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carbonyl)cyclopropane-1- sulfonamide | 687.6 |
| P-0453 | 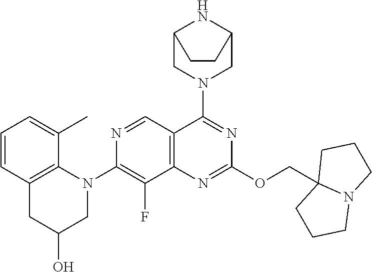 [SMILES: Cc1cccc2c1N(c1ncc3c(N4CC5CCC(C4)N5)nc(OCC45CCCN4CCC5)nc3c1F)CC(O)C2] | 1-(4-((1R,5S)-3,8- diazabicyclo[3.2.1]octan-3-yl)-8- fluoro-2-((tetrahydro-1H- pyrrolizin-7a(5H)- yl)methoxy)pyrido[4,3- d]pyrimidin-7-yl)-8-methyl-1,2,3,4- tetrahydroquinolin-3-ol | 560.4 |
| P-0454 | 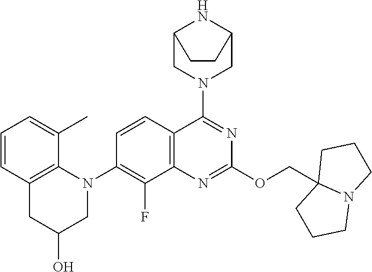 [SMILES: Cc1cccc2c1N(c1ccc3c(N4CC5CCC(C4)N5)nc(OCC45CCCN4CCC5)nc3c1F)CC(O)C2] | 1-(4-((1R,5S)-3,8- diazabicyclo[3.2.1]octan-3-yl)-8- fluoro-2-((tetrahydro-1H- pyrrolizin-7a(5H)- yl)methoxy)quinazolin-7-yl)-8- methyl-1,2,3,4-tetrahydroquinolin- 3-ol | 559.4 |
| P-0455 | 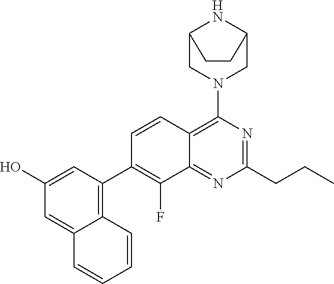 [SMILES: CCCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-propylquinazolin-7- yl)naphthalen-2-ol | 443.5 |
| P-0456 | 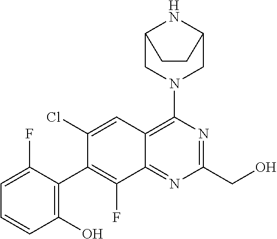 [SMILES: OCc1nc(N2CC3CCC(C2)N3)c2cc(Cl)c(-c3c(O)cccc3F)c(F)c2n1] | 2-(4-((1R,5S)-3,8- diazabicyclo[3.2.1]octan-3-yl)-6- chloro-8-fluoro-2- (hydroxymethyl)quinazolin-7-yl)-3- fluorophenol | 433.2 |
| P-0457 | 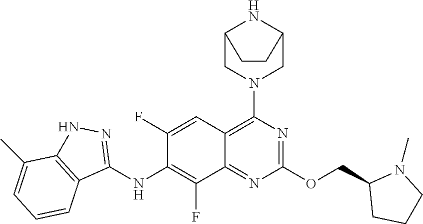 [SMILES: Cc1cccc2c(Nc3c(F)cc4c(N5CC6CCC(C5)N6)nc(OC[C@@H]5CCCN5C)nc4c3F)n[nH]c12] | 4-((1R,5S)-3,8- diazabicyclo[3.2.1]octan-3-yl)-6,8- difluoro-N-(7-methyl-1H-indazol- 3-yl)-2-(((S)-1-methylpyrrolidin-2- yl)methoxy)quinazolin-7-amine | 535.2 |
| P-0458 | 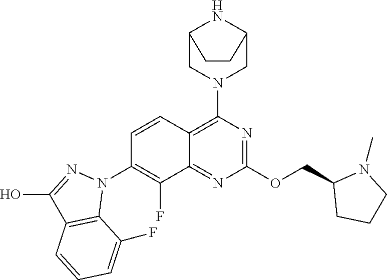 [SMILES: CN1CCC[C@H]1COc1nc(N2CC3CCC(C2)N3)c2ccc(-n3nc(O)c4cccc(F)c43)c(F)c2n1] | 1-(4-((1R,5S)-3,8- diazabicyclo[3.2.1]octan-3-yl)-8- fluoro-2-(((S)-1-methylpyrrolidin- 2-yl)methoxy)quinazolin-7-yl)-7- fluoro-1H-indazol-3-ol | 522.2 |
| P-0459 | 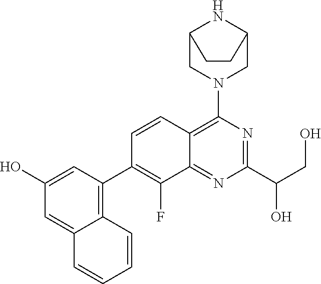 [SMILES: OCC(O)c1nc(N2CC3CCC(C2)N3)c2ccc(-c3cc(O)cc4ccccc34)c(F)c2n1] | 1-(4-((1R,5S)-3,8- diazabicyclo[3.2.1]octan-3-yl)-8- fluoro-7-(3-hydroxynaphthalen-1- yl)quinazolin-2-yl)ethane-1,2-diol | 461.1 |
| P-0460 | 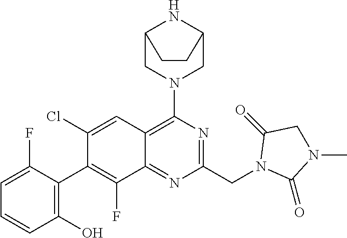 [SMILES: CN1CC(=O)N(Cc2nc(N3CC4CCC(C3)N4)c3cc(Cl)c(-c4c(O)cccc4F)c(F)c3n2)C1=O] | 3-((4-((1R,5S)-3,8- diazabicyclo[3.2.1]octan-3-yl)-6- chloro-8-fluoro-7-(2-fluoro-6- hydroxyphenyl)quinazolin-2- yl)methyl)-1-methylimidazolidine- 2,4-dione | 529.2 |
| P-0461 | 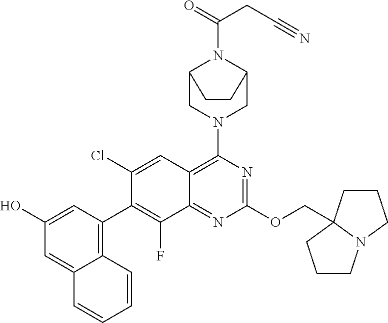 [SMILES: N#CCC(=O)N1C2CCC1CN(c1nc(OCC34CCCN3CCC4)nc3c(F)c(-c4cc(O)cc5ccccc45)c(Cl)cc13)C2] | 3-((1R,5S)-3-(6-chloro-8-fluoro-7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanenitrile | 641.2 |
| P-0462 | 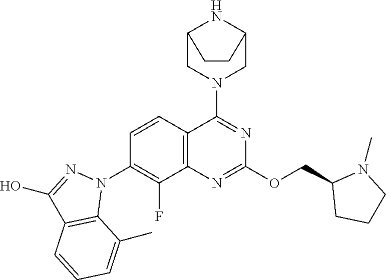 [SMILES: Cc1cccc2c(O)nn(-c3ccc4c(N5CC6CCC(C5)N6)nc(OC[C@@H]5CCCN5C)nc4c3F)c12] | 1-(4-((1R,5S)-3,8- diazabicyclo[3.2.1]octan-3-yl)-8- fluoro-2-(((S)-1-methylpyrrolidin- 2-yl)methoxy)quinazolin-7-yl)-7- methyl-1H-indazol-3-ol | 518.2 |
| P-0463 | 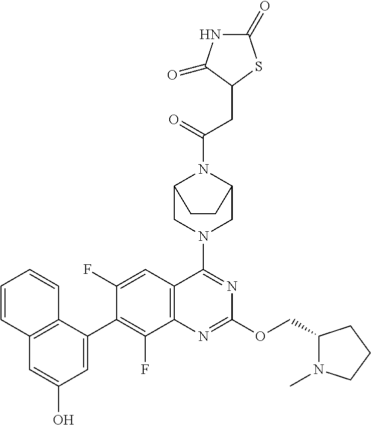 [SMILES: CN1CCC[C@H]1COc1nc(N2CC3CCC(C2)N3C(=O)CC2SC(=O)NC2=O)c2cc(F)c(-c3cc(O)cc4ccccc34)c(F)c2n1] | 5-(2-((1R,5S)-3-(6,8-difluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- oxoethyl)thiazolidine-2,4-dione | 689.3 |
| P-0464 | 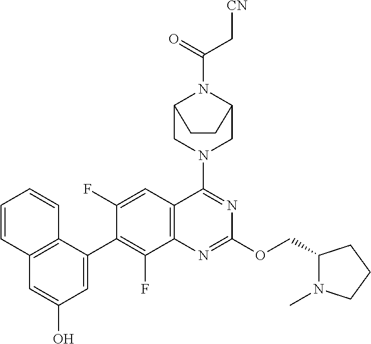 [SMILES: CN1CCC[C@H]1COc1nc(N2CC3CCC(C2)N3C(=O)CC#N)c2cc(F)c(-c3cc(O)cc4ccccc34)c(F)c2n1] | 3-((1R,5S)-3-(6,8-difluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-3- oxopropanenitrile | 599.6 |
| P-0465 | 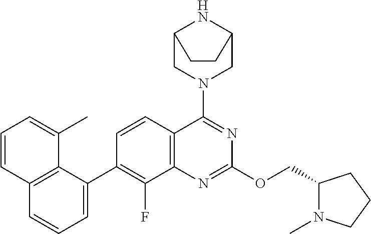 [SMILES: Cc1cccc2cccc(-c3ccc4c(N5CC6CCC(C5)N6)nc(OC[C@@H]5CCCN5C)nc4c3F)c12] | 4-((1R,5S)-3,8- diazabicyclo[3.2.1]octan-3-yl)-8- fluoro-7-(8-methylnaphthalen-1- yl)-2-(((S)-1-methylpyrrolidin-2- yl)methoxy)quinazoline | 512.4 |
| P-0466 | 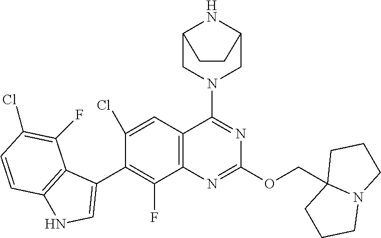 [SMILES: Fc1c(-c2c[nH]c3ccc(Cl)c(F)c23)c(Cl)cc2c(N3CC4CCC(C3)N4)nc(OCC34CCCN3CCC4)nc12] | 4-((1R,5S)-3,8- diazabicyclo[3.2.1]octan-3-yl)-6- chloro-7-(5-chloro-4-fluoro-1H- indol-3-yl)-8-fluoro-2-((tetrahydro- 1H-pyrrolizin-7a(5H)- yl)methoxy)quinazoline | 599.2 |
| P-0467 | 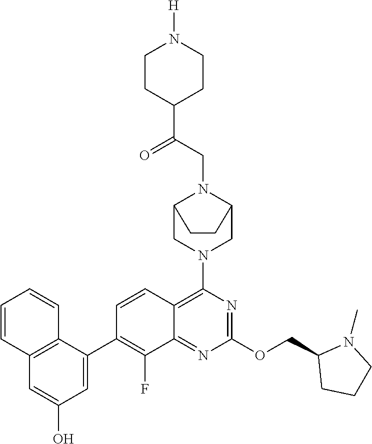 [SMILES: CN1CCC[C@H]1COc1nc(N2CC3CCC(C2)N3CC(=O)C2CCNCC2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-1- (piperidin-4-yl)ethan-1-one | 639.4 |
| P-0468 | 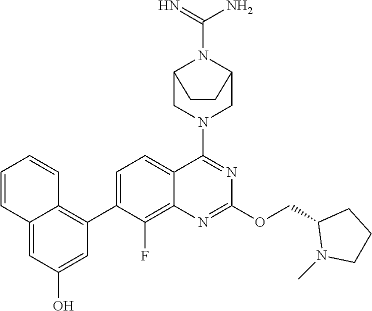 [SMILES: CN1CCC[C@H]1COc1nc(N2CC3CCC(C2)N3C(=N)N)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- carboximidamide | 556 |
| P-0469 | 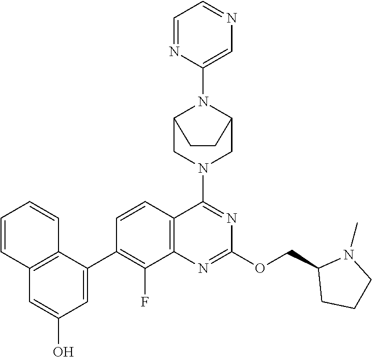 [SMILES: CN1CCC[C@H]1COc1nc(N2CC3CCC(C2)N3c2cnccn2)c2ccc(-c3cc(O)cc4ccccc34)c(F)c2n1] | 4-(8-fluoro-2-(((S)-1- methylpyrrolidin-2-yl)methoxy)-4- ((1R,5S)-8-(pyrazin-2-yl)-3,8- diazabicyclo[3.2.1]octan-3- yl)quinazolin-7-yl)naphthalen-2-ol | 592.6 |
| P-0470 | 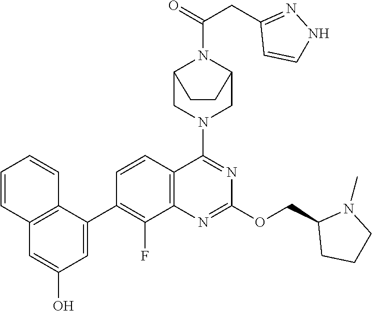 [SMILES: CN1CCC[C@H]1COc1nc(N2CC3CCC(C2)N3C(=O)Cc2cc[nH]n2)c2ccc(-c3cc(O)cc4ccccc34)c(F)c2n1] | 1-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-2- (1H-pyrazol-3-yl)ethan-1-one | 622.3 |
| P-0471 | 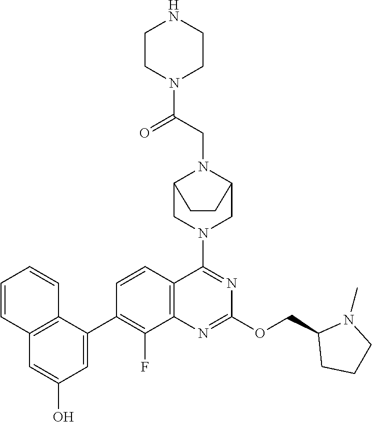 [SMILES: CN1CCC[C@H]1COc1nc(N2CC3CCC(C2)N3CC(=O)N2CCNCC2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-1- (piperazin-1-yl)ethan-1-one | 640.6 |
| P-0472 | 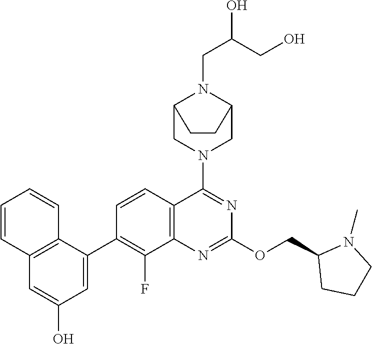 [SMILES: CN1CCC[C@H]1COc1nc(N2CC3CCC(C2)N3CC(O)CO)c2ccc(-c3cc(O)cc4ccccc34)c(F)c2n1] | 3-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8- yl)propane-1,2-diol | 588.4 |
| P-0473 | 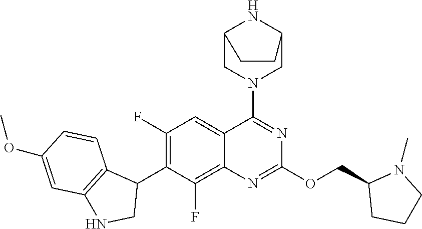 [SMILES: COc1ccc2c(c1)NCC2c1c(F)cc2c(N3CC4CCC(C3)N4)nc(OC[C@@H]3CCCN3C)nc2c1F] | 4-((1R,5S)-3,8- diazabicyclo[3.2.1]octan-3-yl)-6,8- difluoro-7-(6-methoxy-1H-indol-3- yl)-2-(((S)-1-methylpyrrolidin-2- yl)methoxy)quinazoline | 535.3 |
| P-0474 | 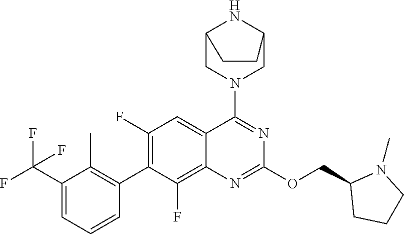 [SMILES: Cc1c(-c2c(F)cc3c(N4CC5CCC(C4)N5)nc(OC[C@@H]4CCCN4C)nc3c2F)cccc1C(F)(F)F] | 4-((1R,5S)-3,8- diazabicyclo[3.2.1]octan-3-yl)-6,8- difluoro-7-(2-methyl-3- (trifluoromethyl)phenyl)-2-(((S)-1- methylpyrrolidin-2- yl)methoxy)quinazoline | 548.5 |
| P-0475 | 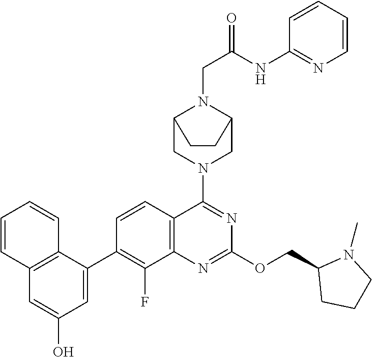 [SMILES: CN1CCC[C@H]1COc1nc(N2CC3CCC(C2)N3CC(=O)Nc2ccccn2)c2ccc(-c3cc(O)cc4ccccc34)c(F)c2n1] | 2-((1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octan-8-yl)-N- (pyridin-2-yl)acetamide | 648.7 |
| P-0476 | 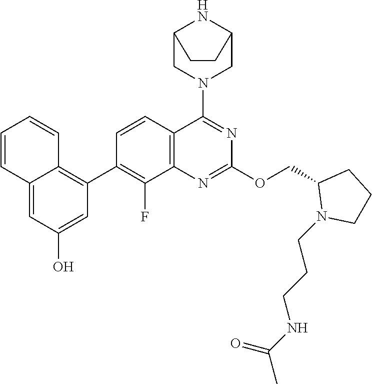 [SMILES: CC(=O)NCCCN1CCC[C@H]1COc1nc(N2CC3CCC(C2)N3)c2ccc(-c3cc(O)cc4ccccc34)c(F)c2n1] | N-(3-((S)-2-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-1- yl)propyl)acetamide | 599.3 |
| P-0477 | 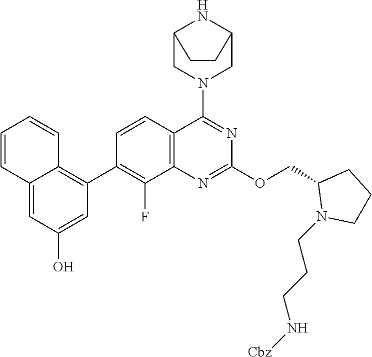 [SMILES: O=C(NCCCN1CCC[C@H]1COc1nc(N2CC3CCC(C2)N3)c2ccc(-c3cc(O)cc4ccccc34)c(F)c2n1)OCc1ccccc1] | benzyl (3-((S)-2-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-1- yl)propyl)carbamate | 691.3 |
| P-0478 | 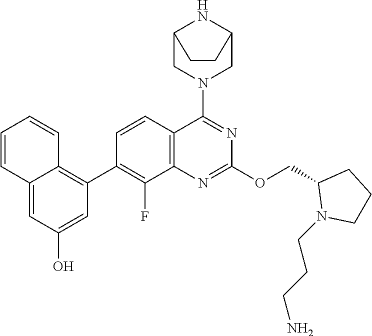 [SMILES: NCCCN1CCC[C@H]1COc1nc(N2CC3CCC(C2)N3)c2ccc(-c3cc(O)cc4ccccc34)c(F)c2n1] | 4-(2-(((S)-1-(3- aminopropyl)pyrrolidin-2- yl)methoxy)-4-((1R,5S)-3,8- diazabicyclo[3.2.1]octan-3-yl)-8- fluoroquinazolin-7-yl)naphthalen- 2-ol | 557.3 |
| P-0479 | 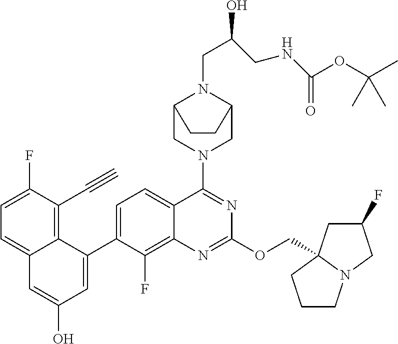 [SMILES: C#Cc1c(F)ccc2cc(O)cc(-c3ccc4c(N5CC6CCC(C5)N6C[C@@H](O)CNC(=O)OC(C)(C)C)nc(OC[C@@]56CCCN5C[C@H](F)C6)nc4c3F)c12] | tert-butyl ((S)-3-((1R,5S)-3-(7-(8- ethynyl-7-fluoro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)carbamate | 772.0 |
| P-0480 | 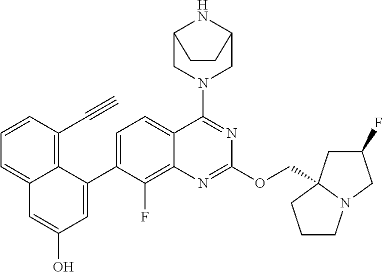 [SMILES: C#Cc1cccc2cc(O)cc(-c3ccc4c(N5CC6CCC(C5)N6)nc(OC[C@@]56CCCN5C[C@H](F)C6)nc4c3F)c12] | tert-butyl ((S)-3-((1R,5S)-3-(7-(8- ethynyl-7-fluoro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)carbamate | 581.0 |
| P-0481 | 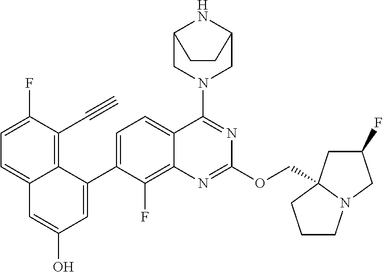 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-ethynyl-6-fluoronaphthalen-2-ol | 599.0 |
| P-0482 | 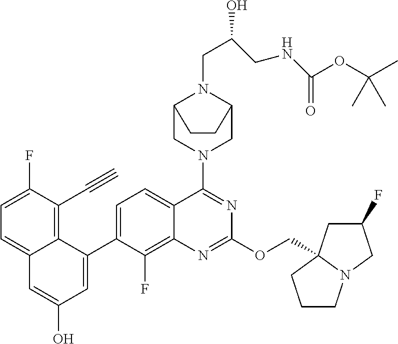 [SMILES: C#Cc1c(F)ccc2cc(O)cc(-c3ccc4c(N5CC6CCC(C5)N6C[C@H](O)CNC(=O)OC(C)(C)C)nc(OC[C@@]56CCCN5C[C@H](F)C6)nc4c3F)c12] | tert-butyl ((R)-3-((1R,5S)-3-(7-(8- ethynyl-7-fluoro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)carbamate | 772.0 |
| P-0483 | 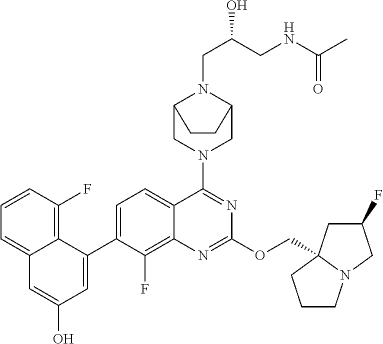 [SMILES: CC(=O)NC[C@@H](O)CN1C2CCC1CN(c1nc(OC[C@@]34CCCN3C[C@H](F)C4)nc3c(F)c(-c4cc(O)cc5cccc(F)c45)ccc13)C2] | N-((R)-3-((1R,5S)-3-(8-fluoro-7-(8- fluoro-3-hydroxynaphthalen-1-yl)-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)acetamide | 690.7 |
| P-0484 | 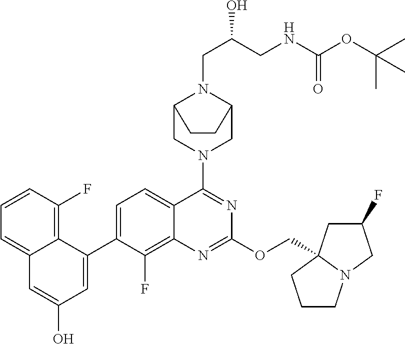 [SMILES: CC(C)(C)OC(=O)NC[C@@H](O)CN1C2CCC1CN(c1nc(OC[C@@]34CCCN3C[C@H](F)C4)nc3c(F)c(-c4cc(O)cc5cccc(F)c45)ccc13)C2] | tert-butyl ((R)-3-((1R,5S)-3-(8-fluoro- 7-(8-fluoro-3-hydroxynaphthalen-1- yl)-2-(((2R,7a S)-2-fluorotetrahydro- 1H-pyrrolizin-7a(5H)- yl)methoxy)quinazolin-4-yl)-3,8- diazabicyclo[3.2.1]octan-8-yl)-2- hydroxypropyl)carbamate | 748.8 |
| P-0485 | 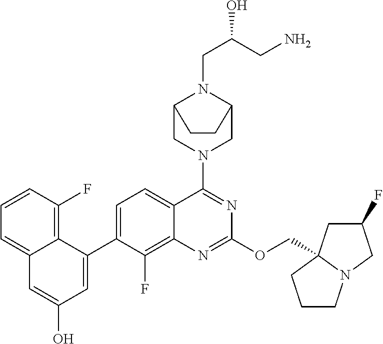 [SMILES: NC[C@@H](O)CN1C2CCC1CN(c1nc(OC[C@@]34CCCN3C[C@H](F)C4)nc3c(F)c(-c4cc(O)cc5cccc(F)c45)ccc13)C2] | 4-(4-((1R,5S)-8-((R)-3-amino-2- hydroxypropyl)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-fluoronaphthalen-2-ol | 648.7 |
| P-0486 | 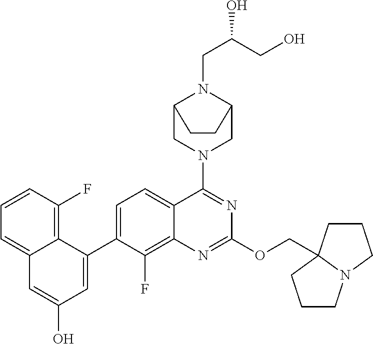 [SMILES: OC[C@@H](O)CN1C2CCC1CN(c1nc(OCC34CCCN3CCC4)nc3c(F)c(-c4cc(O)cc5cccc(F)c45)ccc13)C2] | (S)-3-((1R,5S)-3-(8-fluoro-7-(8- fluoro-3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)propane-1,2-diol | 631.7 |
| P-0487 | 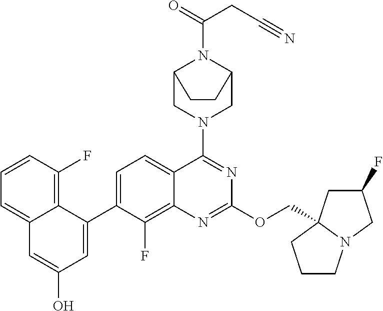 [SMILES: N#CCC(=O)N1C2CCC1CN(c1nc(OC[C@@]34CCCN3C[C@H](F)C4)nc3c(F)c(-c4cc(O)cc5cccc(F)c45)ccc13)C2] | 3-((1R,5S)-3-(8-fluoro-7-(8-fluoro-3- hydroxynaphthalen-1-yl)-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-3- oxopropanenitrile | 642.7 |
| P-0488 | 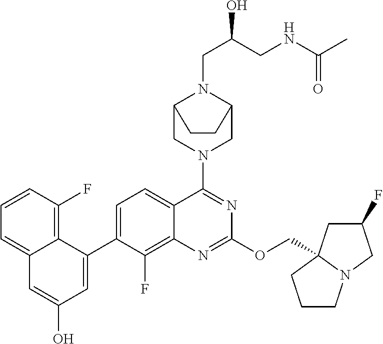 [SMILES: CC(=O)NC[C@H](O)CN1C2CCC1CN(c1nc(OC[C@@]34CCCN3C[C@H](F)C4)nc3c(F)c(-c4cc(O)cc5cccc(F)c45)ccc13)C2] | N-((S)-3-((1R,5S)-3-(8-fluoro-7-(8- fluoro-3-hydroxynaphthalen-1 -yl)-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)acetamide | 690.0 |
| P-0489 | 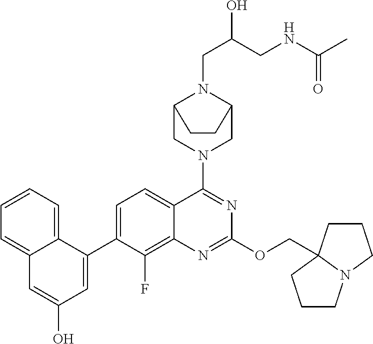 [SMILES: CC(=O)NCC(O)CN1C2CCC1CN(c1nc(OCC34CCCN3CCC4)nc3c(F)c(-c4cc(O)cc5ccccc45)ccc13)C2] | N-(3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- hydroxypropyl)acetamide | 654.0 |
| P-0490 | 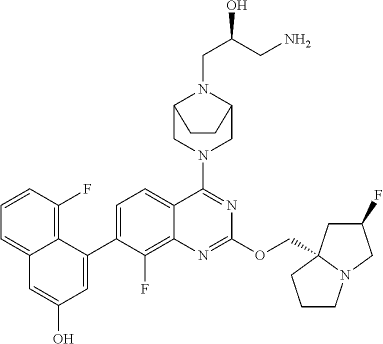 [SMILES: NC[C@H](O)CN1C2CCC1CN(c1nc(OC[C@@]34CCCN3C[C@H](F)C4)nc3c(F)c(-c4cc(O)cc5cccc(F)c45)ccc13)C2] | 4-(4-((1R,5S)-8-((S)-3-amino-2- hydroxypropyl)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-fluoronaphthalen-2-ol | 649.0 |
| P-0491 | 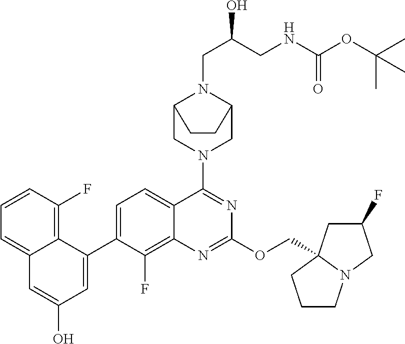 [SMILES: CC(C)(C)OC(=O)NC[C@H](O)CN1C2CCC1CN(c1nc(OC[C@@]34CCCN3C[C@H](F)C4)nc3c(F)c(-c4cc(O)cc5cccc(F)c45)ccc13)C2] | tert-butyl ((S)-3-((1R,5S)-3-(8-fluoro- 7-(8-fluoro-3-hydroxynaphthalen-1- yl)-2-(((2R,7a S)-2-fluorotetrahydro- 1H-pyrrolizin-7a(5H)- yl)methoxy)quinazolin-4-yl)-3,8- diazabicyclo[3.2.1]octan-8-yl)-2- hydroxypropyl)carbamate | 749.0 |
| P-0492 | 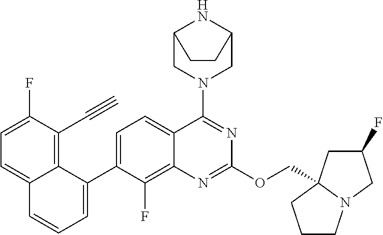 [SMILES: C#Cc1c(F)ccc2cccc(-c3ccc4c(N5CC6CCC(C5)N6)nc(OC[C@@]56CCCN5C[C@H](F)C6)nc4c3F)c12] | 4-((1R,5S)-3,8- diazabicyclo[3.2.1]octan-3-yl)-7-(8- ethynyl-7-fluoronaphthalen-1-yl)-8- fluoro-2-(((2R,7aS)-2- fluorotetrahydro-1H-pyrrolizin- 7a(5H)-yl)methoxy)quinazoline | 584.0 |
| P-0493 | 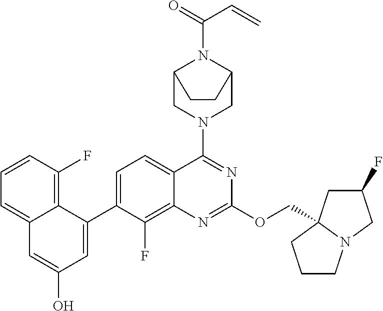 [SMILES: C=CC(=O)N1C2CCC1CN(c1nc(OC[C@@]34CCCN3C[C@H](F)C4)nc3c(F)c(-c4cc(O)cc5cccc(F)c45)ccc13)C2] | 1-((1R,5S)-3-(8-fluoro-7-(8-fluoro-3- hydroxynaphthalen-1-yl)-2- (((2R,7aS)-2-fluorotetrahydro-1H- pyrrolizin-7a(5H)- yl)methoxy)quinazolin-4-yl)-3,8- diazabicyclo[3.2.1]octan-8-yl)prop-2- en-1-one | 630.0 |
| P-0494 | 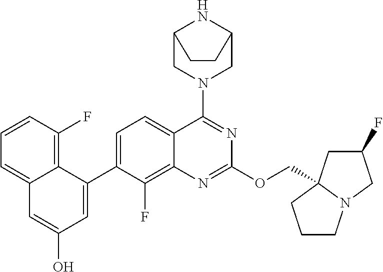 [SMILES: Oc1cc(-c2ccc3c(N4CC5CCC(C4)N5)nc(OC[C@@]45CCCN4C[C@H](F)C5)nc3c2F)c2c(F)cccc2c1] | 4-(4-((1R,5S)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-fluoronaphthalen-2-ol | 576.0 |
| P-0495 | 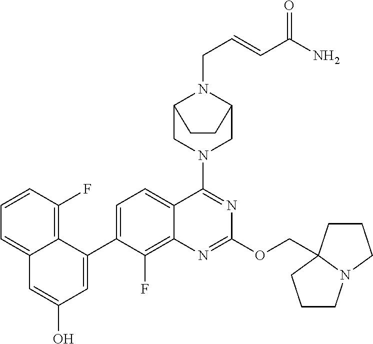 [SMILES: NC(=O)/C=C/CN1C2CCC1CN(c1nc(OCC34CCCN3CCC4)nc3c(F)c(-c4cc(O)cc5cccc(F)c45)ccc13)C2] | (E)-4-((1R,5S)-3-(8-fluoro-7-(8- fluoro-3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)but-2- enamide | 641.0 |
| P-0496 | 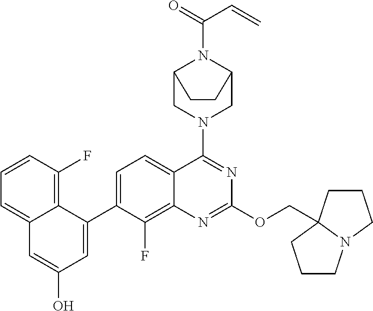 [SMILES: C=CC(=O)N1C2CCC1CN(c1nc(OCC34CCCN3CCC4)nc3c(F)c(-c4cc(O)cc5cccc(F)c45)ccc13)C2] | 1-((1R,5S)-3-(8-fluoro-7-(8-fluoro-3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)prop-2- en-1-one | 612.0 |
| P-0497 | 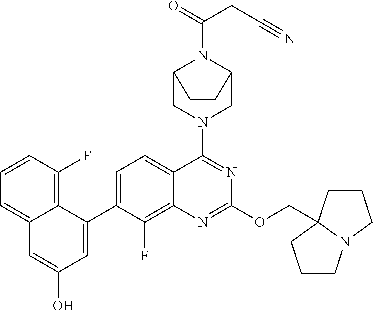 [SMILES: N#CCC(=O)N1C2CCC1CN(c1nc(OCC34CCCN3CCC4)nc3c(F)c(-c4cc(O)cc5cccc(F)c45)ccc13)C2] | 3-((1R,5S)-3-(8-fluoro-7-(8-fluoro-3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanenitrile | 625.0 |
| P-0498 | 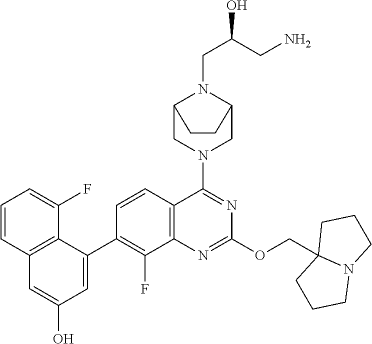 [SMILES: NC[C@H](O)CN1C2CCC1CN(c1nc(OCC34CCCN3CCC4)nc3c(F)c(-c4cc(O)cc5cccc(F)c45)ccc13)C2] | 4-(4-((1R,5S)-8-((S)-3-amino-2- hydroxypropyl)-3,8- diazabicyclo[3.2.1]octan-3-yl)-8- fluoro-2-((tetrahydro-1H-pyrrolizin- 7a(5H)-yl)methoxy)quinazolin-7-yl)- 5-fluoronaphthalen-2-ol | 630.7 |
| P-0499 | 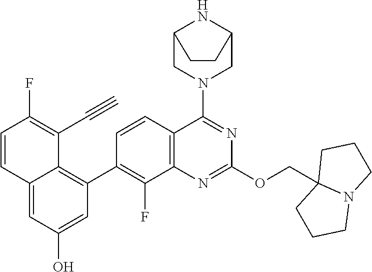 [SMILES: C#Cc1c(F)ccc2cc(O)cc(-c3ccc4c(N5CC6CCC(C5)N6)nc(OCC56CCCN5CCC6)nc4c3F)c12] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 5-ethynyl-6-fluoronaphthalen-2-ol | 581.6 |
| P-0500 | 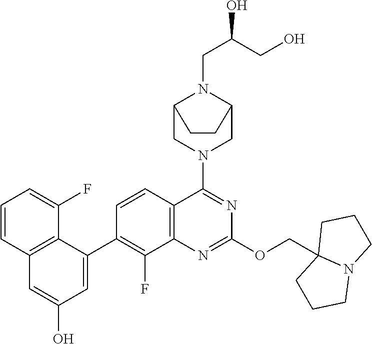 [SMILES: OC[C@H](O)CN1C2CCC1CN(c1nc(OCC34CCCN3CCC4)nc3c(F)c(-c4cc(O)cc5cccc(F)c45)ccc13)C2] | (R)-3-((1R,5S)-3-(8-fluoro-7-(8- fluoro-3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)propane-1,2-diol | 631.7 |
| P-0501 | 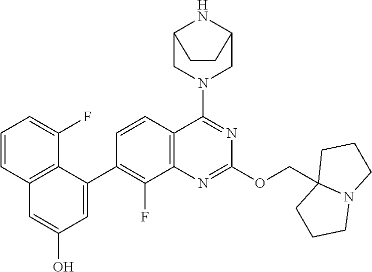 [SMILES: Oc1cc(-c2ccc3c(N4CC5CCC(C4)N5)nc(OCC45CCCN4CCC5)nc3c2F)c2c(F)cccc2c1] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 5-fluoronaphthalen-2-ol | 557.6 |
| P-0502 | 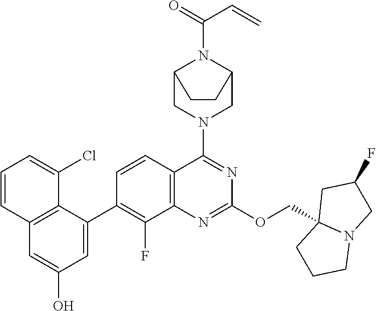 [SMILES: C=CC(=O)N1C2CCC1CN(c1nc(OC[C@@]34CCCN3C[C@H](F)C4)nc3c(F)c(-c4cc(O)cc5cccc(Cl)c45)ccc13)C2] | 1-((1R,5S)-3-(7-(8-chloro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)prop-2- en-1-one | 647.1 |
| P-0503 | 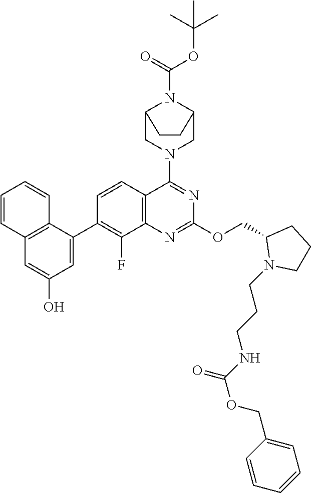 [SMILES: CC(C)(C)OC(=O)N1C2CCC1CN(c1nc(OC[C@@H]3CCCN3CCCNC(=O)OCc3ccccc3)nc3c(F)c(-c4cc(O)cc5ccccc45)ccc13)C2] | tert-butyl (1R,5S)-3-(2-(((S)-1-(3- (((benzyloxy)carbonyl)amino)propyl) pyrrolidin-2-yl)methoxy)-8-fluoro-7- (3-hydroxynaphthalen-1- yl)quinazolin-4-yl)-3,8- diazabicyclo[3.2.1]octane-8- carboxylate | 791.4 |
| P-0504 | 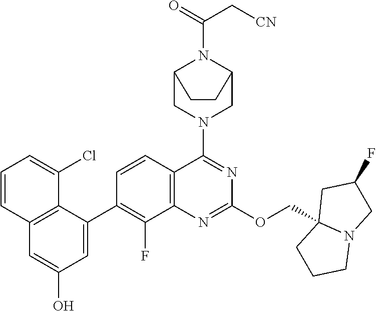 [SMILES: N#CCC(=O)N1C2CCC1CN(c1nc(OC[C@@]34CCCN3C[C@H](F)C4)nc3c(F)c(-c4cc(O)cc5cccc(Cl)c45)ccc13)C2] | 3-((1R,5S)-3-(7-(8-chloro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-3- oxopropanenitrile | 659.2 |
| P-0505 | 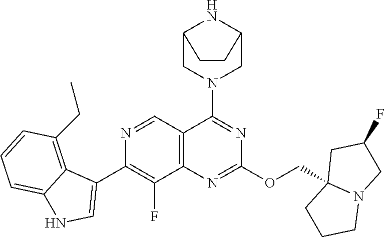 [SMILES: CCc1cccc2[nH]cc(-c3ncc4c(N5CC6CCC(C5)N6)nc(OC[C@@]56CCCN5C[C@H](F)C6)nc4c3F)c12] | 4-((1R,5S)-3,8- diazabicyclo[3.2.1]octan-3-yl)-7-(4- ethyl-1H-indol-3-yl)-8-fluoro-2- (((2R,7aS)-2-fluorotetrahydro-1H- pyrrolizin-7a(5H)- yl)methoxy)pyrido[4,3-d]pyrimidine | 560.3 |
| P-0506 | 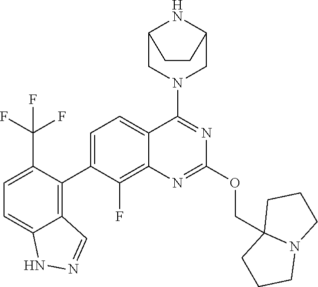 [SMILES: Fc1c(-c2c(C(F)(F)F)ccc3[nH]ncc23)ccc2c(N3CC4CCC(C3)N4)nc(OCC34CCCN3CCC4)nc12] | 4-((1R,5S)-3,8- diazabicyclo[3.2.1]octan-3-yl)-8- fluoro-2-((tetrahydro-1H-pyrrolizin- 7a(5H)-yl)methoxy)-7-(5- (trifluoromethyl)-1H-indazol-4- yl)quinazoline | 581.3 |
| P-0507 | 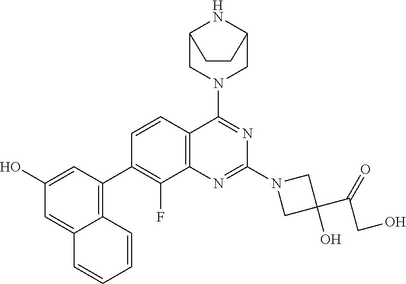 [SMILES: O=C(CO)C1(O)CN(c2nc(N3CC4CCC(C3)N4)c3ccc(-c4cc(O)cc5ccccc45)c(F)c3n2)C1] | 1-(1-(4-((1R,5S)-3,8- diazabicyclo[3.2.1]octan-3-yl)-8- fluoro-7-(3-hydroxynaphthalen-1- yl)quinazolin-2-yl)-3- hydroxyazetidin-3-yl)-2- hydroxyethan-1-one | 530.2 |
| P-0508 | 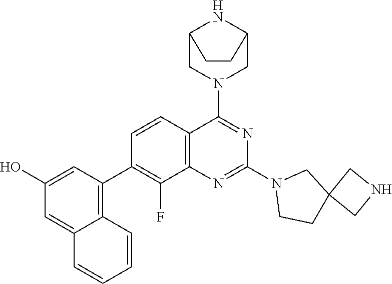 [SMILES: Oc1cc(-c2ccc3c(N4CC5CCC(C4)N5)nc(N4CCC5(CNC5)C4)nc3c2F)c2ccccc2c1] | 4-(4-((1R,5S)-3,8- diazabicyclo[3.2.1]octan-3-yl)-8- fluoro-2-(2,6-diazaspiro[3.4]octan-6- yl)quinazolin-7-yl)naphthalen-2-ol | 511.3 |
| P-0509 | 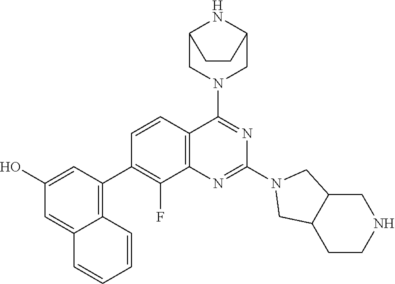 [SMILES: Oc1cc(-c2ccc3c(N4CC5CCC(C4)N5)nc(N4CC5CCNCC5C4)nc3c2F)c2ccccc2c1] | 4-(4-((1R,5S)-3,8- diazabicyclo[3.2.1]octan-3-yl)-8- fluoro-2-(octahydro-2H-pyrrolo[3,4- c]pyridin-2-yl)quinazolin-7- yl)naphthalen-2-ol | 525.2 |
| P-0510 | 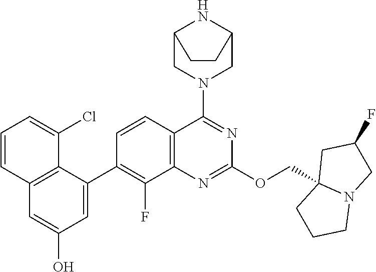 [SMILES: Oc1cc(-c2ccc3c(N4CC5CCC(C4)N5)nc(OC[C@@]45CCCN4C[C@H](F)C5)nc3c2F)c2c(Cl)cccc2c1] | 4-(4-((1R,5S)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-chloronaphthalen-2-ol | 592.2 |
| P-0511 | 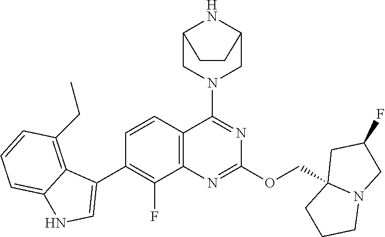 [SMILES: CCc1cccc2[nH]cc(-c3ccc4c(N5CC6CCC(C5)N6)nc(OC[C@@]56CCCN5C[C@H](F)C6)nc4c3F)c12] | 4-((1R,5S)-3,8- diazabicyclo[3.2.1]octan-3-yl)-7-(4- ethyl-1H-indol-3-yl)-8-fluoro-2- (((2R,7aS)-2-fluorotetrahydro-1H- pyrrolizin-7a(5H)- yl)methoxy)quinazoline | 559.3 |
| P-0512 | 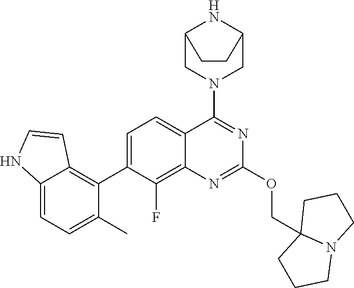 [SMILES: Cc1ccc2[nH]ccc2c1-c1ccc2c(N3CC4CCC(C3)N4)nc(OCC34CCCN3CCC4)nc2c1F] | 5-((1R,5S)-bicyclo[3.2.1]octan-3-yl)- 7-(2-(hexahydropentalen-3a(1H)- yl)ethyl)-1-methyl-2-(5-methyl-1H- inden-4-yl)naphthalene | 526.3 |
| P-0513 | 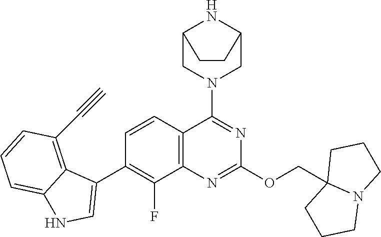 [SMILES: C#Cc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethynyl-1H-indol-3-yl)-8-fluoro-2- ((tetrahydro-1H-pyrrolizin-7a(5H)- yl)methoxy)quinazoline | 537.2 |
| P-0514 | 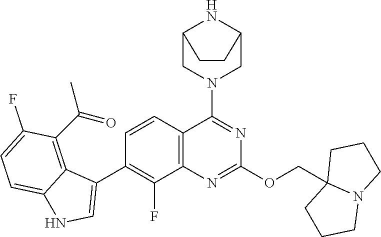 [SMILES: CC(=O)c1c(F)ccc2[nH]cc(-c3ccc4c(N5CC6CCC(C5)N6)nc(OCC56CCCN5CCC6)nc4c3F)c12] | 5-((1R,5S)-bicyclo[3.2.1]octan-3-yl)- 7-(2-(hexahydropentalen-3a(1H)- yl)ethyl)-1-methyl-2-(5-methyl-4- (prop-1-en-2-yl)-1H-inden-3- yl)naphthalene | 573.2 |
| P-0515 | 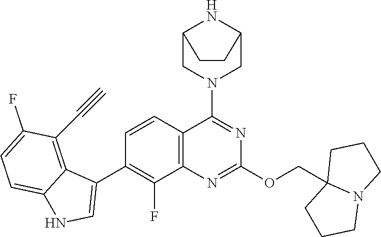 [SMILES: C#Cc1c(F)ccc2[nH]cc(-c3ccc4c(N5CC6CCC(C5)N6)nc(OCC56CCCN5CCC6)nc4c3F)c12] | 5-((1R,5S)-bicyclo[3.2.1]octan-3-yl)- 2-(4-ethynyl-5-methyl-1H-inden-3- yl)-7-(2-(hexahydropentalen-3a(1H)- yl)ethyl)-1-methylnaphthalene | 555.2 |
| P-0516 | 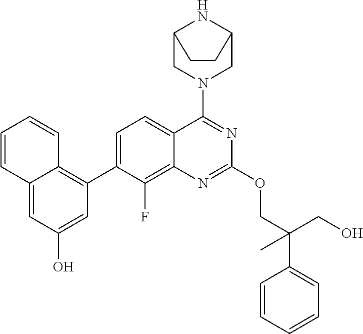 [SMILES: CC(CO)(COc1nc(N2CC3CCC(C2)N3)c2ccc(-c3cc(O)cc4ccccc34)c(F)c2n1)c1ccccc1] | 4-(4-((1R,5S)-3,8- diazabicyclo[3.2.1]octan-3-yl)-8- fluoro-2-(3-hydroxy-2-methyl-2- phenylpropoxy)quinazolin-7- yl)naphthalen-2-ol | 564.3 |
| P-0517 | 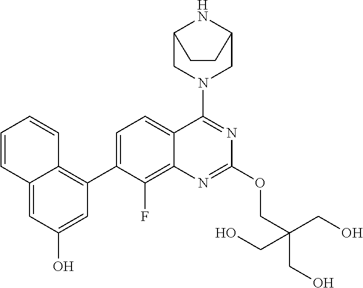 [SMILES: OCC(CO)(CO)COc1nc(N2CC3CCC(C2)N3)c2ccc(-c3cc(O)cc4ccccc34)c(F)c2n1] | 5′-((1R,5S)-bicyclo[3.2.1]octan-3-yl)- 7′-(3,3-diethylpentyl)-1′,3-dimethyl- 1,2′-binaphthalene | 534.2 |
| P-0518 | 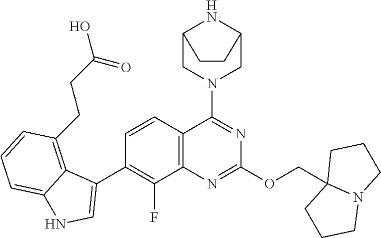 [SMILES: O=C(O)CCc1cccc2[nH]cc(-c3ccc4c(N5CC6CCC(C5)N6)nc(OCC56CCCN5CCC6)nc4c3F)c12] | 3-(3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indol-4-yl)propanoic acid | 585.3 |
| P-0519 | 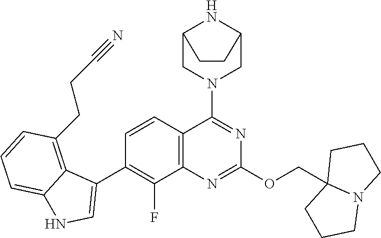 [SMILES: N#CCCc1cccc2[nH]cc(-c3ccc4c(N5CC6CCC(C5)N6)nc(OCC56CCCN5CCC6)nc4c3F)c12] | 3-(3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indol-4-yl)propanenitrile | 566.3 |
| P-0520 | 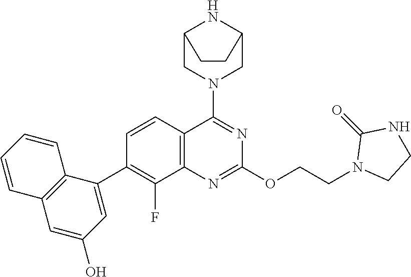 [SMILES: O=C1NCCN1CCOc1nc(N2CC3CCC(C2)N3)c2ccc(-c3cc(O)cc4ccccc34)c(F)c2n1] | 1-(2-((4-((1R,5S)-3,8- diazabicyclo[3.2.1]octan-3-yl)-8- fluoro-7-(3-hydroxynaphthalen-1- yl)quinazolin-2- yl)oxy)ethyl)imidazolidin-2-one | 527.2 |
| P-0521 | 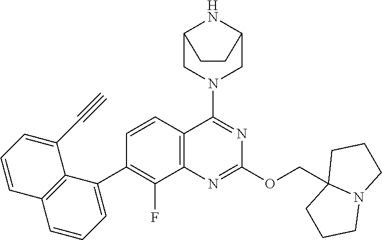 [SMILES: C#Cc1cccc2cccc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(8- ethynylnaphthalen-1-yl)-8-fluoro-2- ((tetrahydro-1H-pyrrolizin-7a(5H)- yl)methoxy)quinazoline | 548.4 |
| P-0522 | 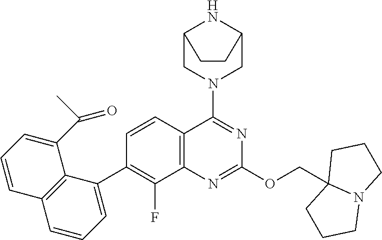 [SMILES: CC(=O)c1cccc2cccc(-c3ccc4c(N5CC6CCC(C5)N6)nc(OCC56CCCN5CCC6)nc4c3F)c12] | 1-(8-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7- yl)naphthalen-1-yl)ethan-1-one | 566.3 |
| P-0523 | 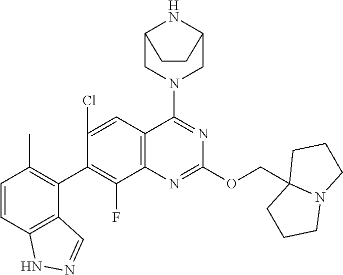 [SMILES: Cc1ccc2[nH]ncc2c1-c1c(Cl)cc2c(N3CC4CCC(C3)N4)nc(OCC34CCCN3CCC4)nc2c1F] | 4-((1R,5S)-3,8- diazabicyclo[3.2.1]octan-3-yl)-6- chloro-8-fluoro-7-(5-methyl-1H- indazol-4-yl)-2-((tetrahydro-1H- pyrrolizin-7a(5H)- yl)methoxy)quinazoline | 562.2 |
| P-0524 | 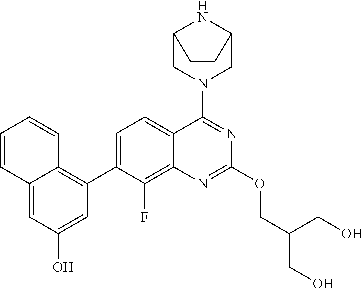 [SMILES: OCC(CO)COc1nc(N2CC3CCC(C2)N3)c2ccc(-c3cc(O)cc4ccccc34)c(F)c2n1] | 2-(((4-((1R,5S)-3,8- diazabicyclo[3.2.1]octan-3-yl)-8- fluoro-7-(3-hydroxynaphthalen-1- yl)quinazolin-2- yl)oxy)methyl)propane-1,3-diol | 504.2 |
| P-0525 | 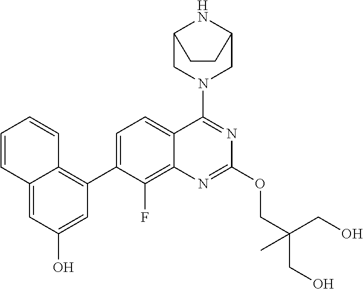 [SMILES: CC(CO)(CO)COc1nc(N2CC3CCC(C2)N3)c2ccc(-c3cc(O)cc4ccccc34)c(F)c2n1] | 2-(((4-((1R,5S)-3,8- diazabicyclo[3.2.1]octan-3-yl)-8- fluoro-7-(3-hydroxynaphthalen-1- yl)quinazolin-2-yl)oxy)methyl)-2- methylpropane-1,3-diol | 518.2 |
| P-0526 | 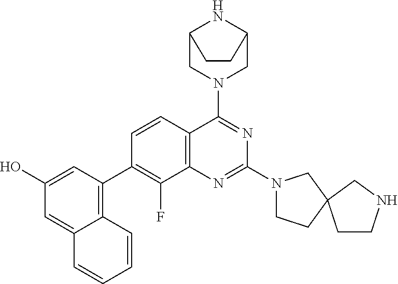 [SMILES: Oc1cc(-c2ccc3c(N4CC5CCC(C4)N5)nc(N4CCC5(CCNC5)C4)nc3c2F)c2ccccc2c1] | 4-(4-((1R,5S)-3,8- diazabicyclo[3.2.1]octan-3-yl)-8- fluoro-2-(2,7-diazaspiro[4.4]nonan-2- yl)quinazolin-7-yl)naphthalen-2-ol | 525.3 |
| P-0527 | 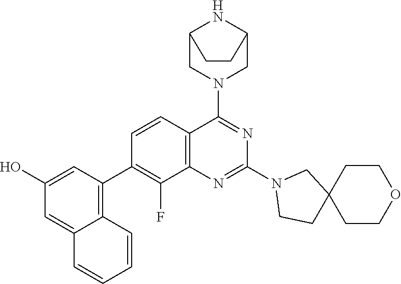 [SMILES: Oc1cc(-c2ccc3c(N4CC5CCC(C4)N5)nc(N4CCC5(CCOCC5)C4)nc3c2F)c2ccccc2c1] | 4-(4-((1R,5S)-3,8- diazabicyclo[3.2.1]octan-3-yl)-8- fluoro-2-(8-oxa-2-azaspiro[4.5]decan- 2-yl)quinazolin-7-yl)naphthalen-2-ol | 540.2 |
| P-0528 | 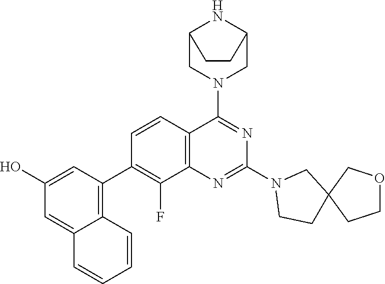 [SMILES: Oc1cc(-c2ccc3c(N4CC5CCC(C4)N5)nc(N4CCC5(CCOC5)C4)nc3c2F)c2ccccc2c1] | 4-(4-((1R,5S)-3,8- diazabicyclo[3.2.1]octan-3-yl)-8- fluoro-2-(2-oxa-7-azaspiro[4.4]nonan- 7-yl)quinazolin-7-yl)naphthalen-2-ol | 526.3 |
| P-0529 | 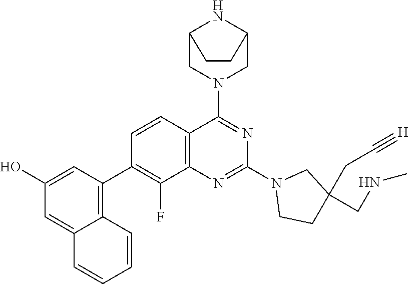 | 2-(1-(4-((1R,5S)-3,8- diazabicyclo[3.2.1]octan-3-yl)-8- fluoro-7-(3-hydroxynaphthalen-1- yl)quinazolin-2-yl)-3- ((methylamino)methyl)pyrrolidin-3- yl)acetonitrile | 552.3 |
| P-0530 | 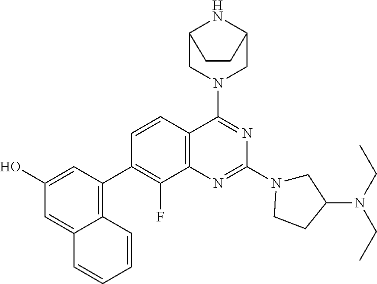 [SMILES: CCN(CC)C1CCN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (diethylamino)pyrrolidin-1-yl)-8- fluoroquinazolin-7-yl)naphthalen-2- ol | 541.3 |
| P-0531 | 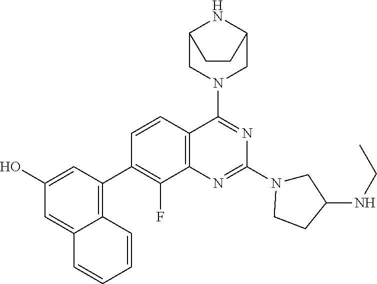 [SMILES: CCNC1CCN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (ethylamino)pyrrolidin-1-yl)-8- fluoroquinazolin-7-yl)naphthalen-2- ol | 513.3 |
| P-0532 | 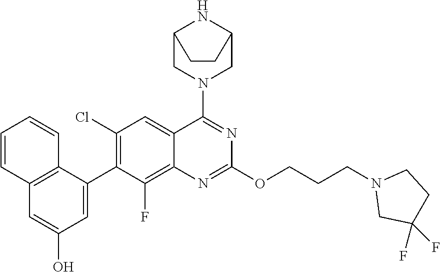 [SMILES: Oc1cc(-c2c(Cl)cc3c(N4CC5CCC(C4)N5)nc(OCCCN4CCC(F)(F)C4)nc3c2F)c2ccccc2c1] | 4-(4-((1R,5S)-3,8- diazabicyclo[3.2.1]octan-3-yl)-6- chloro-2-(3-(3,3-difluoropyrrolidin-1- yl)propoxy)-8-fluoroquinazolin-7- yl)naphthalen-2-ol | 598.2 |
| P-0533 | 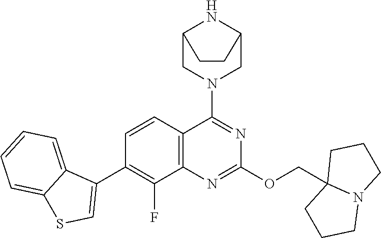 [SMILES: Fc1c(-c2csc3ccccc23)ccc2c(N3CC4CCC(C3)N4)nc(OCC34CCCN3CCC4)nc12] | 7-(benzo[b]thiophen-3-yl)-4-((1R,5S)- 3,8-diazabicyclo[3.2.1]octan-3-yl)-8- fluoro-2-((tetrahydro-1H-pyrrolizin- 7a(5H)-yl)methoxy)quinazoline | 530.2 |
| P-0534 | 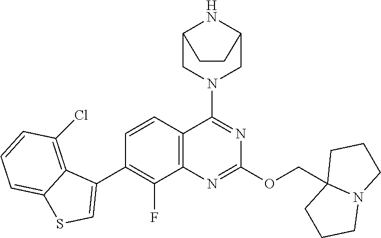 [SMILES: Fc1c(-c2csc3cccc(Cl)c23)ccc2c(N3CC4CCC(C3)N4)nc(OCC34CCCN3CCC4)nc12] | 4-((1R,5S)-3,8- diazabicyclo[3.2.1]octan-3-yl-7-(4- chlorobenzo[b]thiophen-3-yl)-8- fluoro-2-((tetrahydro-1H-pyrrolizin- 7a(5H)-yl)methoxy)quinazoline | 566.2 |
| P-0535 | 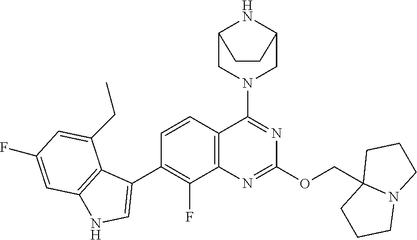 [SMILES: CCc1cc(F)cc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-6-fluoro-1H-indol-3-yl)-8- fluoro-2-((tetrahydro-1H-pyrrolizin- 7a(5H)-yl)methoxy)quinazoline | 559.7 |
| P-0536 | 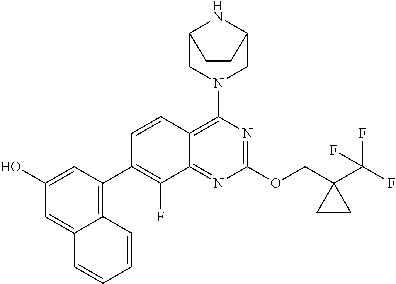 [SMILES: Oc1cc(-c2ccc3c(N4CC5CCC(C4)N5)nc(OCC4(C(F)(F)F)CC4)nc3c2F)c2ccccc2c1] | 4-(4-((1R,5S)-3,8- diazabicyclo[3.2.1]octan-3-yl)-8- fluoro-2-((1- (trifluoromethyl)cyclopropyl)methoxy) quinazolin-7-yl)naphthalen-2-ol | 539.2 |
| P-0537 | 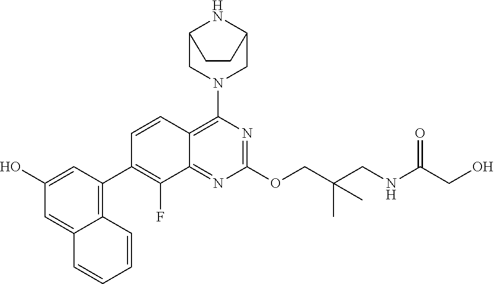 [SMILES: CC(C)(CNC(=O)CO)COc1nc(N2CC3CCC(C2)N3)c2ccc(-c3cc(O)cc4ccccc34)c(F)c2n1] | N-(3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropyl)-2-hydroxyacetamide | 560.6 |
| P-0538 | 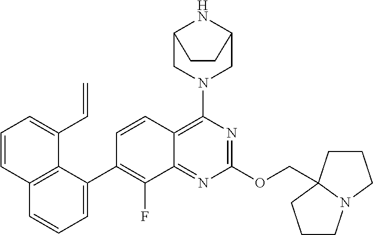 [SMILES: C=Cc1cccc2cccc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-2-((tetrahydro-1H-pyrrolizin- 7a(5H)-yl)methoxy)-7-(8- vinylnaphthalen-1-yl)quinazoline | 550.3 |
| P-0539 | 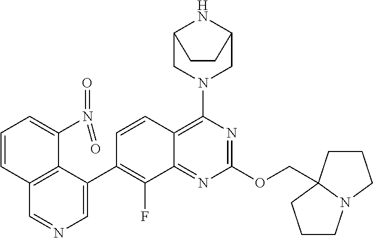 [SMILES: O=[N+]([O-])c1cccc2cncc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(5-nitroisoquinolin-4-yl)-2- ((tetrahydro-1H-pyrrolizin-7a(5H)- yl)methoxy)quinazoline | 570.6 |
| P-0540 | 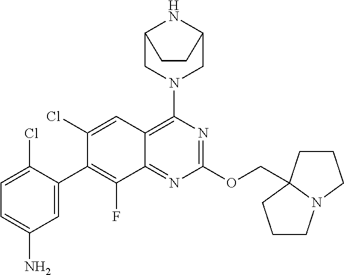 [SMILES: Nc1ccc(Cl)c(-c2c(Cl)cc3c(N4CC5CCC(C4)N5)nc(OCC45CCCN4CCC5)nc3c2F)c1] | 3-(4-((1R,5S)-3,8- diazabicyclo[3.2.1]octan-3-yl)-6- chloro-8-fluoro-2-((tetrahydro-1H- pyrrolizin-7a(5H)- yl)methoxy)quinazolin-7-yl)-4- chloroaniline | 520.8 |
| P-0541 | 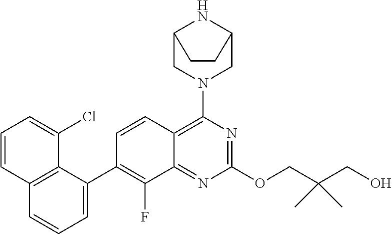 [SMILES: CC(C)(CO)COc1nc(N2CC3CCC(C2)N3)c2ccc(-c3cccc4cccc(Cl)c34)c(F)c2n1] | 3-((4-((1R,5S)-3,8- diazabicyclo[3.2.1]octan-3-yl)-7-(8- chloronaphthalen-1-yl)-8- fluoroquinazolin-2-yl)oxy)-2,2- dimethylpropan-1-ol | 521.2 |
| P-0542 | 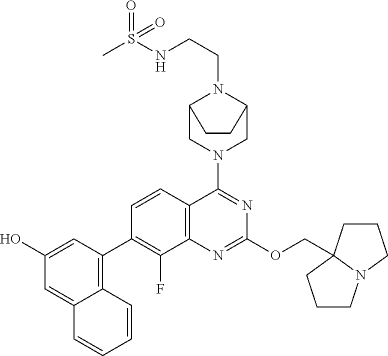 [SMILES: CS(=O)(=O)NCCN1C2CCC1CN(c1nc(OCC34CCCN3CCC4)nc3c(F)c(-c4cc(O)cc5ccccc45)ccc13)C2] | N-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethyl)methanesulfonamide | 661.3 |
| P-0543 | 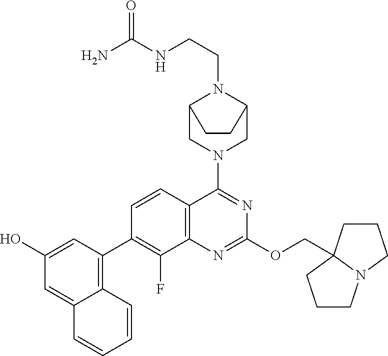 [SMILES: NC(=O)NCCN1C2CCC1CN(c1nc(OCC34CCCN3CCC4)nc3c(F)c(-c4cc(O)cc5ccccc45)ccc13)C2] | 1-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethyl)urea | 626.3 |
| P-0544 | 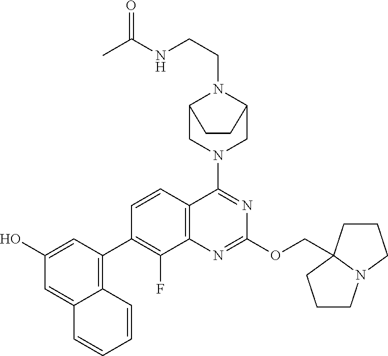 [SMILES: CC(=O)NCCN1C2CCC1CN(c1nc(OCC34CCCN3CCC4)nc3c(F)c(-c4cc(O)cc5ccccc45)ccc13)C2] | N-(2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethyl)acetamide | 625.4 |
| P-0545 | 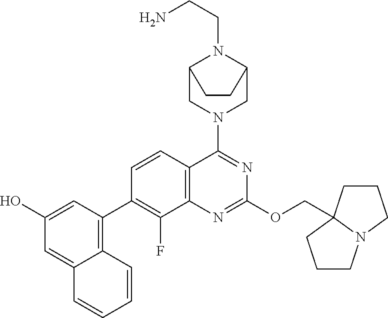 [SMILES: NCCN1C2CCC1CN(c1nc(OCC34CCCN3CCC4)nc3c(F)c(-c4cc(O)cc5ccccc45)ccc13)C2] | 4-(4-((1R,5S)-8-(2-aminoethyl)-3,8- diazabicyclo[3.2.1]octan-3-yl)-8- fluoro-2-((tetrahydro-1H-pyrrolizin- 7a(5H)-yl)methoxy)quinazolin-7- yl)naphthalen-2-ol | 583.3 |
| P-0546 | 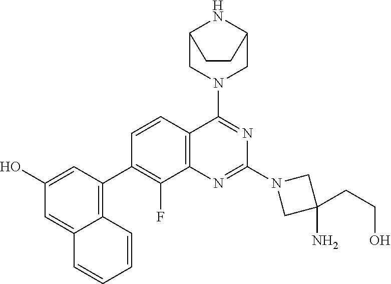 [SMILES: NC1(CCO)CN(c2nc(N3CC4CCC(C3)N4)c3ccc(-c4cc(O)cc5ccccc45)c(F)c3n2)C1] | 4-(2-(3-amino-3-(2- hydroxyethyl)azetidin-1-yl)-4- ((1R,5S)-3,8- diazabicyclo[3.2.1]octan-3-yl)-8- fluoroquinazolin-7-yl)naphthalen-2- ol | 515.3 |
| P-0547 | 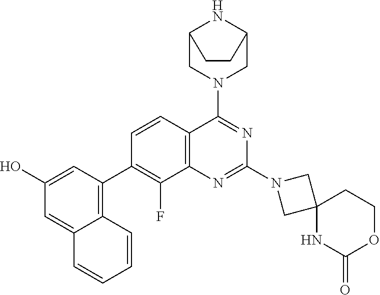 [SMILES: O=C1NC2(CCO1)CN(c1nc(N3CC4CCC(C3)N4)c3ccc(-c4cc(O)cc5ccccc45)c(F)c3n1)C2] | 2-(4-((1R,5S)-3,8- diazabicyclo[3.2.1]octan-3-yl)-8- fluoro-7-(3-hydroxynaphthalen-1- yl)quinazolin-2-yl)-7-oxa-2,5- diazaspiro[3.5]nonan-6-one | 541.2 |
| P-0548 | 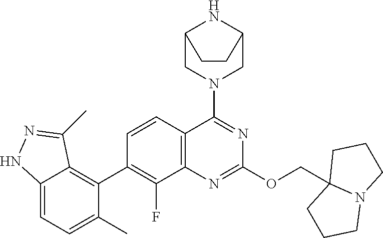 [SMILES: Cc1ccc2[nH]nc(C)c2c1-c1ccc2c(N3CC4CCC(C3)N4)nc(OCC34CCCN3CCC4)nc2c1F] | 4-((1R,5S)-3,8- diazabicyclo[3.2.1]octan-3-yl)-7-(3,5- dimethyl-1H-indazol-4-yl)-8-fluoro-2- ((tetrahydro-1H-pyrrolizin-7a(5H)- yl)methoxy)quinazoline | 542.3 |
| P-0549 | 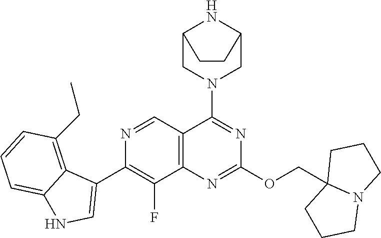 [SMILES: CCc1cccc2[nH]cc(-c3ncc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-1H-indol-3-yl)-8-fluoro-2- ((tetrahydro-1H-pyrrolizin-7a(5H)- yl)methoxy)pyrido[4,3-d]pyrimidine | 542.3 |
| P-0550 | 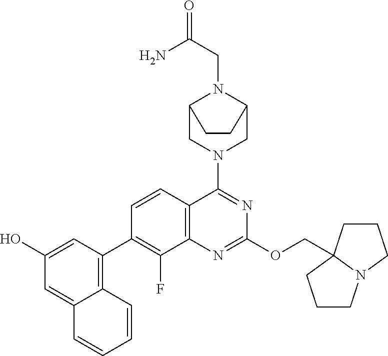 [SMILES: NC(=O)CN1C2CCC1CN(c1nc(OCC34CCCN3CCC4)nc3c(F)c(-c4cc(O)cc5ccccc45)ccc13)C2] | 2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)acetamide | 597.3 |
| P-0551 | 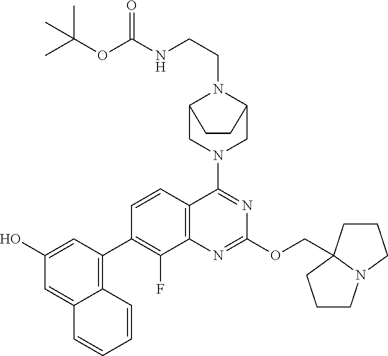 [SMILES: CC(C)(C)OC(=O)NCCN1C2CCC1CN(c1nc(OCC34CCCN3CCC4)nc3c(F)c(-c4cc(O)cc5ccccc45)ccc13)C2] | tert-butyl (2-((1R,5S)-3-(8-fluoro-7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)ethyl)carbamate | 683.3 |
| P-0552 | 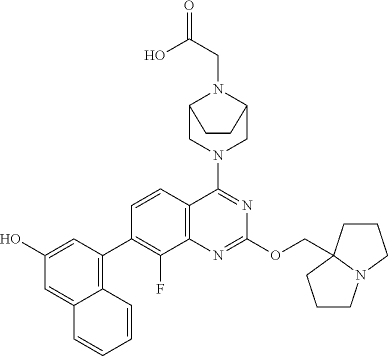 [SMILES: O=C(O)CN1C2CCC1CN(c1nc(OCC34CCCN3CCC4)nc3c(F)c(-c4cc(O)cc5ccccc45)ccc13)C2] | 2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)acetic acid | 598.3 |
| P-0553 | 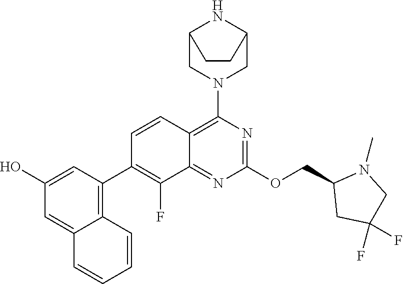 [SMILES: CN1CC(F)(F)C[C@H]1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2- (((S)-4,4-difluoro-1-methylpyrrolidin- 2-yl)methoxy)-8-fluoroquinazolin-7- yl)naphthalen-2-ol | 550.3 |
| P-0554 | 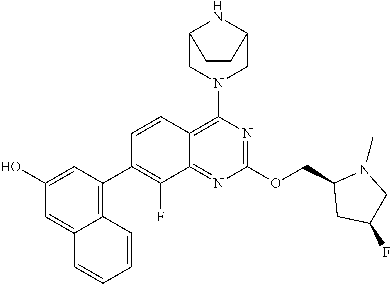 [SMILES: CN1C[C@@H](F)C[C@H]1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(((2S,4S)-4-fluoro-1- methylpyrrolidin-2- yl)methoxy)quinazolin-7- yl)naphthalen-2-ol | 532.3 |
| P-0555 | 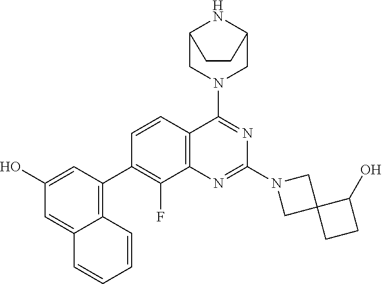 [SMILES: Oc1cc(-c2ccc3c(N4CC5CCC(C4)N5)nc(N4CC5(CCC5O)C4)nc3c2F)c2ccccc2c1] | 2-(4-((1R,5S)-3,8- diazabicyclo[3.2.1]octan-3-yl)-8- fluoro-7-(3-hydroxynaphthalen-1- yl)quinazolin-2-yl)-2- azaspiro[3.3]heptan-5-ol | 512.3 |
| P-0556 | 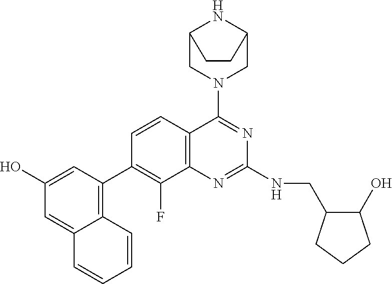 [SMILES: Oc1cc(-c2ccc3c(N4CC5CCC(C4)N5)nc(NCC4CCCC4O)nc3c2F)c2ccccc2c1] | 4-(4-((1R,5S)-3,8- diazabicyclo[3.2.1]octan-3-yl)-8- fluoro-2-(((2- hydroxycyclopentyl)methyl)amino) quinazolin-7-yl)naphthalen-2-ol | 514.3 |
| P-0557 | 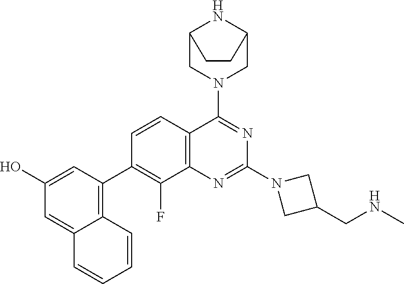 [SMILES: CNCC1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3- ((methylamino)methyl)azetidin-1- yl)quinazolin-7-yl)naphthalen-2-ol | 499.3 |
| P-0558 | 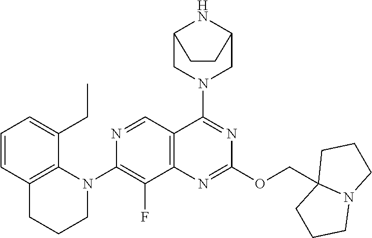 [SMILES: CCc1cccc2c1N(c1ncc3c(N4CC5CCC(C4)N5)nc(OCC45CCCN4CCC5)nc3c1F)CCC2] | 4-((1R,5S)-3,8- diazabicyclo[3.2.1]octan-3-yl)-7-(8- ethyl-3,4-dihydroquinolin-1(2H)-yl)- 8-fluoro-2-((tetrahydro-1H-pyrrolizin- 7a(5H)-yl)methoxy)pyrido[4,3- d]pyrimidine | 558.3 |
| P-0559 | 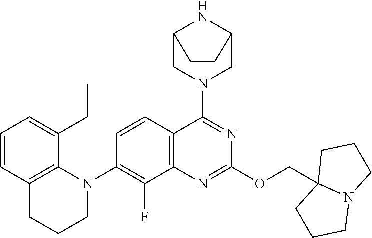 [SMILES: CCc1cccc2c1N(c1ccc3c(N4CC5CCC(C4)N5)nc(OCC45CCCN4CCC5)nc3c1F)CCC2] | 4-((1R,5S)-3,8- diazabicyclo[3.2.1]octan-3-yl)-7-(8- ethyl-3,4-dihydroquinolin-1(2H)-yl)- 8-fluoro-2-((tetrahydro-1H-pyrrolizin- 7a(5H)-yl)methoxy)quinazoline | 557.3 |
| P-0560 | 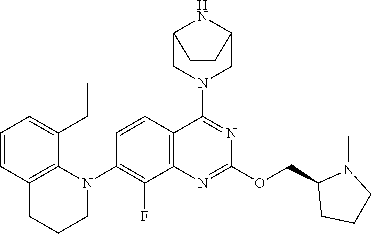 [SMILES: CCc1cccc2c1N(c1ccc3c(N4CC5CCC(C4)N5)nc(OC[C@@H]4CCCN4C)nc3c1F)CCC2] | 4-((1R,5S)-3,8- diazabicyclo[3.2.1]octan-3-yl)-7-(8- ethyl-3,4-dihydroquinolin-1(2H)-yl)- 8-fluoro-2-(((S)-1-methylpyrrolidin-2- yl)methoxy)quinazoline | 531.3 |
| P-0561 | 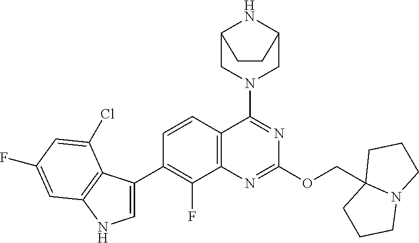 [SMILES: Fc1cc(Cl)c2c(-c3ccc4c(N5CC6CCC(C5)N6)nc(OCC56CCCN5CCC6)nc4c3F)c[nH]c2c1] | 4-((1R,5S)-3,8- diazabicyclo[3.2.1]octan-3-yl)-7-(4- chloro-6-fluoro-1H-indol-3-yl)-8- fluoro-2-((tetrahydro-1H-pyrrolizin- 7a(5H)-yl)methoxy)quinazoline | 566.1 |
| P-0562 | 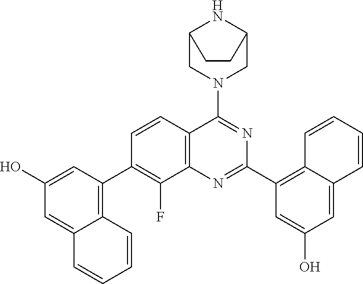 [SMILES: Oc1cc(-c2nc(N3CC4CCC(C3)N4)c3ccc(-c4cc(O)cc5ccccc45)c(F)c3n2)c2ccccc2c1] | 4,4′-(4-((1R,5S)-3,8- diazabicyclo[3.2.1]octan-3-yl)-8- fluoroquinazoline-2,7- diyl)bis(naphthalen-2-ol) | 543.3 |
| P-0563 | 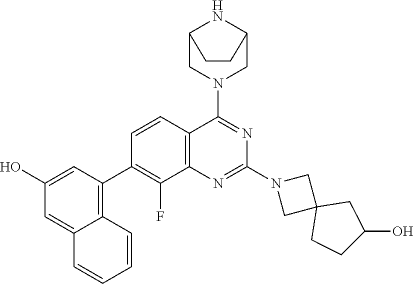 [SMILES: Oc1cc(-c2ccc3c(N4CC5CCC(C4)N5)nc(N4CC5(CCC(O)C5)C4)nc3c2F)c2ccccc2c1] | 2-(4-((1R,5S)-3,8- diazabicyclo[3.2.1]octan-3-yl)-8- fluoro-7-(3-hydroxynaphthalen-1- yl)quinazolin-2-yl)-2- azaspiro[3.4]octan-6-ol | 526.3 |
| P-0564 | 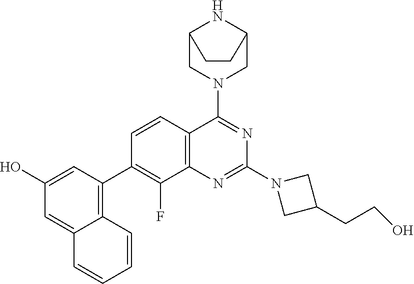 [SMILES: OCCC1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3-(2-hydroxyethyl)azetidin- 1-yl)quinazolin-7-yl)naphthalen-2-ol | 5003 |
| P-0565 | 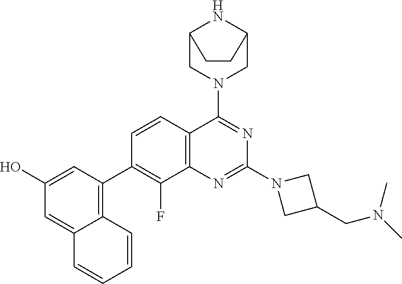 [SMILES: CN(C)CC1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- ((dimethylamino)methyl)azetidin-1- yl)-8-fluoroquinazolin-7- yl)naphthalen-2-ol | 513.3 |
| P-0566 | 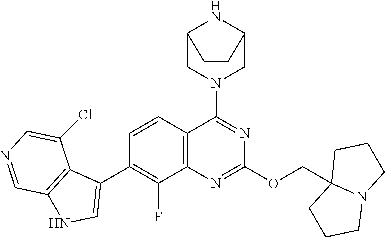 [SMILES: Fc1c(-c2c[nH]c3cncc(Cl)c23)ccc2c(N3CC4CCC(C3)N4)nc(OCC34CCCN3CCC4)nc12] | 4-((1R,5S)-3,8- diazabicyclo[3.2.1]octan-3-yl)-7-(4- chloro-1H-pyrrolo[2,3-c]pyridin-3- yl)-8-fluoro-2-((tetrahydro-1H- pyrrolizin-7a(5H)- yl)methoxy)quinazoline | 548.3 |
| P-0567 | 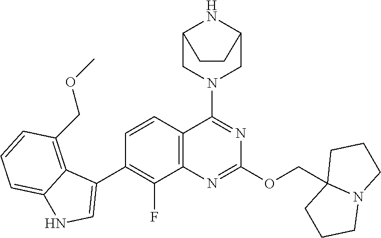 [SMILES: COCc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(4-(methoxymethyl)-1H- indol-3-yl)-2-((tetrahydro-1H- pyrrolizin-7a(5H)- yl)methoxy)quinazoline | 557.3 |
| P-0568 | 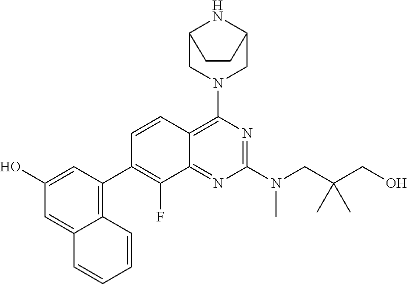 [SMILES: CN(CC(C)(C)CO)c1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-((3-hydroxy-2,2- dimethylpropyl)(methyl)amino)quinaz- olin-7-yl)naphthalen-2-ol | 516.6 |
| P-0569 | 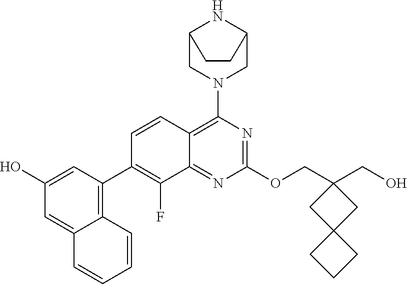 [SMILES: OCC1(COc2nc(N3CC4CCC(C3)N4)c3ccc(-c4cc(O)cc5ccccc45)c(F)c3n2)CC2(CCC2)C1] | 4-(4-((1R,5S)-3,8- diazabicyclo[3.2.1]octan-3-yl)-8- fluoro-2-((2- (hydroxymethyl)spiro[3.3]heptan-2- yl)methoxy)quinazolin-7- yl)naphthalen-2-ol | 555.3 |
| P-0570 | 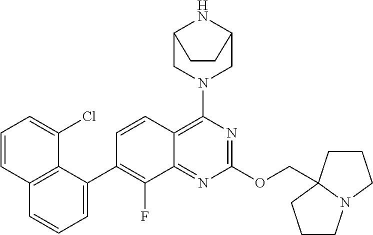 [SMILES: Fc1c(-c2cccc3cccc(Cl)c23)ccc2c(N3CC4CCC(C3)N4)nc(OCC34CCCN3CCC4)nc12] | 4-((1R,5S)-3,8- diazabicyclo[3.2.1]octan-3-yl)-7-(8- chloronaphthalen-1-yl)-8-fluoro-2- ((tetrahydro-1H-pyrrolizin-7a(5H)- yl)methoxy)quinazoline | 558.6 |
| P-0571 | 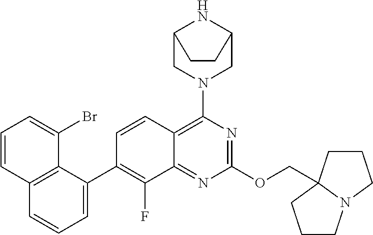 [SMILES: Fc1c(-c2cccc3cccc(Br)c23)ccc2c(N3CC4CCC(C3)N4)nc(OCC34CCCN3CCC4)nc12] | 4-((1R,5S)-3,8- diazabicyclo[3.2.1]octan-3-yl)-7-(8- bromonaphthalen-1-yl)-8-fluoro-2- ((tetrahydro-1H-pyrrolizin-7a(5H)- yl)methoxy)quinazoline | 602.5 |
| P-0572 | 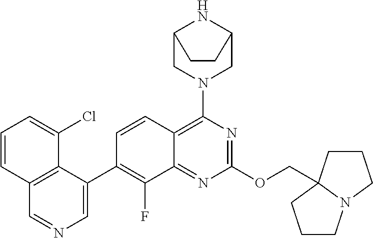 [SMILES: Fc1c(-c2cncc3cccc(Cl)c23)ccc2c(N3CC4CCC(C3)N4)nc(OCC34CCCN3CCC4)nc12] | 4-((1R,5S)-3,8- diazabicyclo[3.2.1]octan-3-yl)-7-(5- chloroisoquinolin-4-yl)-8-fluoro-2- ((tetrahydro-1H-pyrrolizin-7a(5H)- yl)methoxy)quinazoline | 559.6 |
| P-0573 | 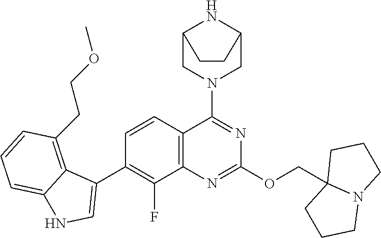 [SMILES: COCCc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(4-(2-methoxyethyl)-1H- indol-3-yl)-2-((tetrahydro-1H- pyrrolizin-7a(5H)- yl)methoxy)quinazoline | 571.3 |
| P-0574 | 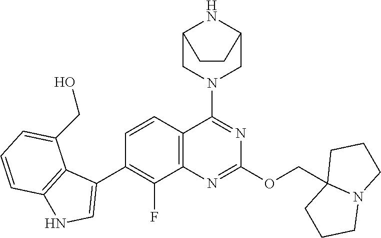 [SMILES: OCc1cccc2[nH]cc(-c3ccc4c(N5CC6CCC(C5)N6)nc(OCC56CCCN5CCC6)nc4c3F)c12] | (3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indol-4-yl)methanol | 543.2 |
| P-0575 | 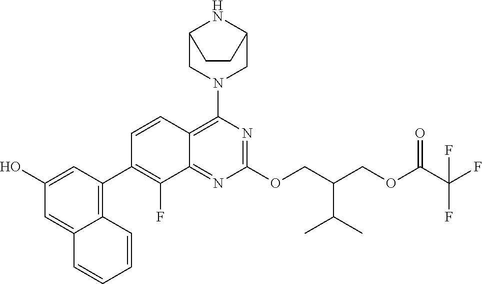 [SMILES: CC(C)C(COC(=O)C(F)(F)F)COc1nc(N2CC3CCC(C2)N3)c2ccc(-c3cc(O)cc4ccccc34)c(F)c2n1] | 2-(((4-((1R,5S)-3,8- diazabicyclo[3.2.1]octan-3-yl)-8- fluoro-7-(3-hydroxynaphthalen-1- yl)quinazolin-2-yl)oxy)methyl)-3- methylbutyl 2,2,2-trifluoroacetate | 613.2 |
| P-0576 | 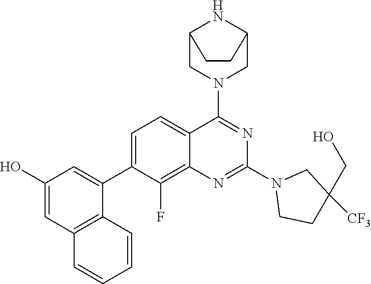 [SMILES: OCC1(C(F)(F)F)CCN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3-(hydroxymethyl)-3- (trifluoromethyl)pyrrolidin-1- yl)quinazolin-7-yl)naphthalen-2-ol | 568.2 |
| P-0577 | 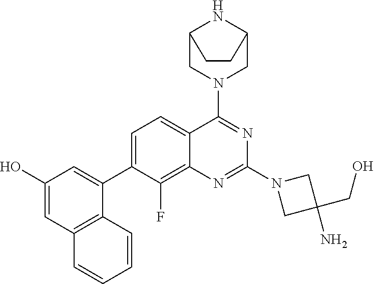 [SMILES: NC1(CO)CN(c2nc(N3CC4CCC(C3)N4)c3ccc(-c4cc(O)cc5ccccc45)c(F)c3n2)C1] | 4-(2-(3-amino-3- (hydroxymethyl)azetidin-1-yl)-4- ((1R,5S)-3,8- diazabicyclo[3.2.1]octan-3-yl)-8- fluoroquinazolin-7-yl)naphthalen-2- ol | 502.2 |
| P-0578 | 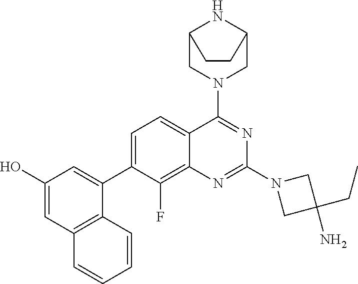 [SMILES: CCC1(N)CN(c2nc(N3CC4CCC(C3)N4)c3ccc(-c4cc(O)cc5ccccc45)c(F)c3n2)C1] | 4-(2-(3-amino-3-ethylazetidin-1-yl)-4- ((1R,5S)-3,8- diazabicyclo[3.2.1]octan-3-yl)-8- fluoroquinazolin-7-yl)naphthalen-2- ol | 499.2 |
| P-0579 | 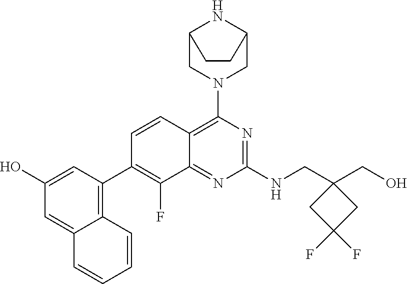 [SMILES: OCC1(CNc2nc(N3CC4CCC(C3)N4)c3ccc(-c4cc(O)cc5ccccc45)c(F)c3n2)CC(F)(F)C1] | 4-(4-((1R,5S)-3,8- diazabicyclo[3.2.1]octan-3-yl)-2- (((3,3-difluoro-1- (hydroxymethyl)cyclobutyl)methyl) amino)-8-fluoroquinazolin-7- yl)naphthalen-2-ol | 550.2 |
| P-0580 | 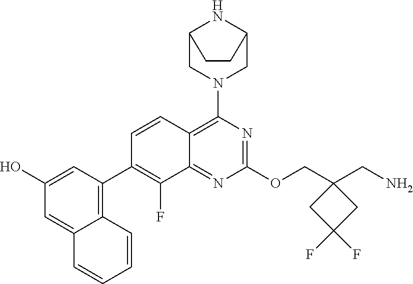 [SMILES: NCC1(COc2nc(N3CC4CCC(C3)N4)c3ccc(-c4cc(O)cc5ccccc45)c(F)c3n2)CC(F)(F)C1] | 4-(2-((1-(aminomethyl)-3,3- difluorocyclobutyl)methoxy)-4- ((1R,5S)-3,8- diazabicyclo[3.2.1]octan-3-yl)-8- fluoroquinazolin-7-yl)naphthalen-2- ol | 550.2 |
| P-0581 | 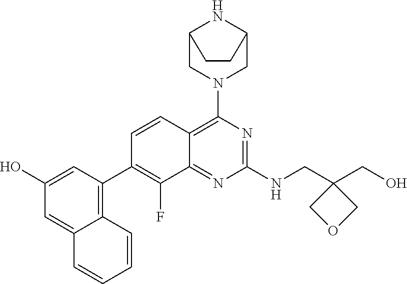 [SMILES: OCC1(CNc2nc(N3CC4CCC(C3)N4)c3ccc(-c4cc(O)cc5ccccc45)c(F)c3n2)COC1] | 4-(4-((1R,5S)-3,8- diazabicyclo[3.2.1]octan-3-yl)-8- fluoro-2-(((3-(hydroxymethyl)oxetan- 3-yl)methyl)amino)quinazolin-7- yl)naphthalen-2-ol | 516.6 |
| P-0582 | 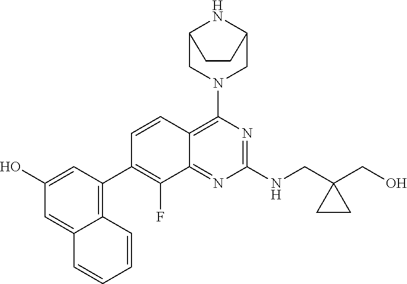 [SMILES: OCC1(CNc2nc(N3CC4CCC(C3)N4)c3ccc(-c4cc(O)cc5ccccc45)c(F)c3n2)CC1] | 4-(4-((1R,5S)-3,8- diazabicyclo[3.2.1]octan-3-yl)-8- fluoro-2-(((1- (hydroxymethyl)cyclopropyl)methyl) amino)quinazolin-7-yl)naphthalen-2-ol | 500.2 |
| P-0583 | 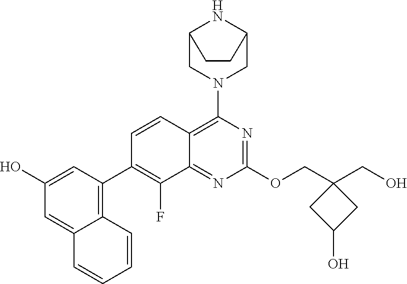 [SMILES: OCC1(COc2nc(N3CC4CCC(C3)N4)c3ccc(-c4cc(O)cc5ccccc45)c(F)c3n2)CC(O)C1] | 4-(4-((1R,5S)-3,8- diazabicyclo[3.2.1]octan-3-yl)-8- fluoro-2-((3-hydroxy-1- (hydroxymethyl)cyclobutyl)methoxy) quinazolin-7-yl)naphthalen-2-ol | 531.2 |
| P-0584 | 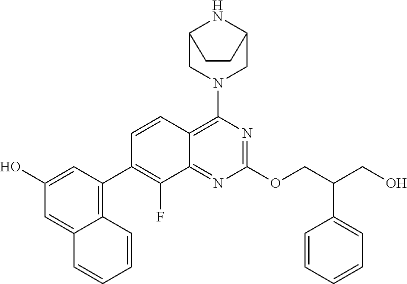 [SMILES: OCC(COc1nc(N2CC3CCC(C2)N3)c2ccc(-c3cc(O)cc4ccccc34)c(F)c2n1)c1ccccc1] | 4-(4-((1R,5S)-3,8- diazabicyclo[3.2.1]octan-3-yl)-8- fluoro-2-(3-hydroxy-2- phenylpropoxy)quinazolin-7- yl)naphthalen-2-ol | 551.2 |
| P-0585 | 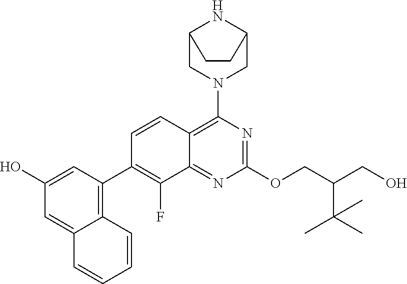 [SMILES: CC(C)(C)C(CO)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2-(hydroxymethyl)-3,3- dimethylbutoxy)quazolin-7- yl)naphthalen-2-ol | 530.3 |
| P-0586 | 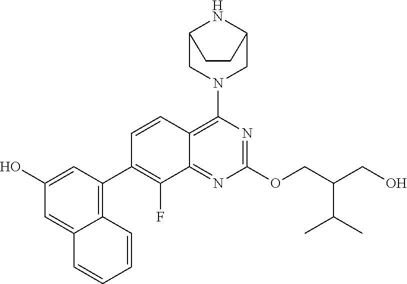 [SMILES: CC(C)C(CO)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2-(hydroxymethyl)-3- methylbutoxy)quinazolin-7- yl)naphthalen-2-ol | 517.3 |
| P-0587 | 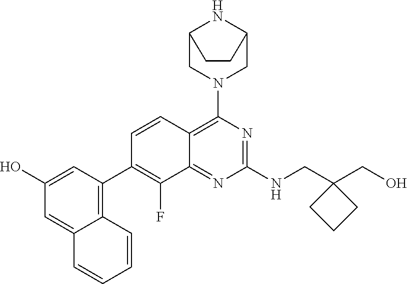 [SMILES: OCC1(CNc2nc(N3CC4CCC(C3)N4)c3ccc(-c4cc(O)cc5ccccc45)c(F)c3n2)CCC1] | 4-(4-((1R,5S)-3,8- diazabicyclo[3.2.1]octan-3-yl)-8- fluoro-2-(((1- (hydroxymethyl)cyclobutyl)methyl) amino)quinazolin-7-yl)naphthalen-2-ol | 514.2 |
| P-0588 | 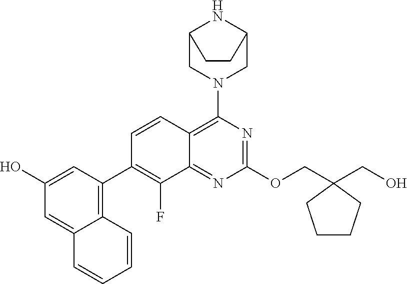 [SMILES: OCC1(COc2nc(N3CC4CCC(C3)N4)c3ccc(-c4cc(O)cc5ccccc45)c(F)c3n2)CCCC1] | 4-(4-((1R,5S)-3,8- diazabicyclo[3.2.1]octan-3-yl)-8- fluoro-2-((1- (hydroxymethyl)cyclopentyl)methoxy) quinazolin-7-yl)naphthalen-2-ol | 529.6 |
| P-0589 | 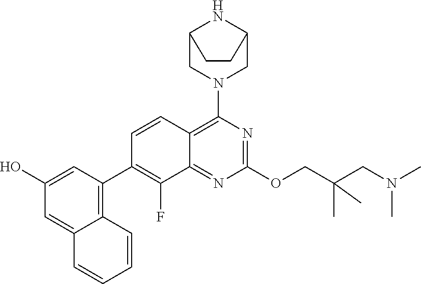 [SMILES: CN(C)CC(C)(C)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-(3- (dimethylamino)-2,2- dimethylpropoxy)-8-fluoroquinazolin- 7-yl)naphthalen-2-ol | 530.3 |
| P-0590 | 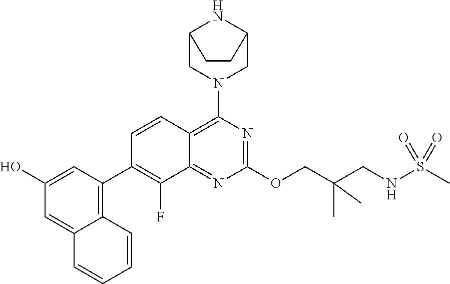 [SMILES: CC(C)(CNS(C)(=O)=O)COc1nc(N2CC3CCC(C2)N3)c2ccc(-c3cc(O)cc4ccccc34)c(F)c2n1] | N-(3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropyl)methanesulfonamide | 588.6 |
| P-0591 | 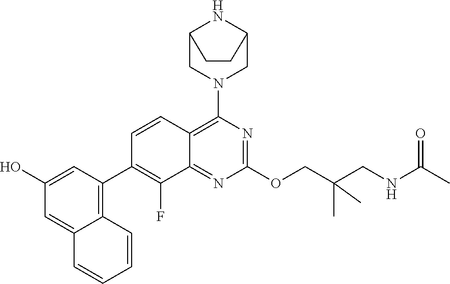 [SMILES: CC(=O)NCC(C)(C)COc1nc(N2CC3CCC(C2)N3)c2ccc(-c3cc(O)cc4ccccc34)c(F)c2n1] | N-(3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropyl)acetamide | 542.2 |
| P-0592 | 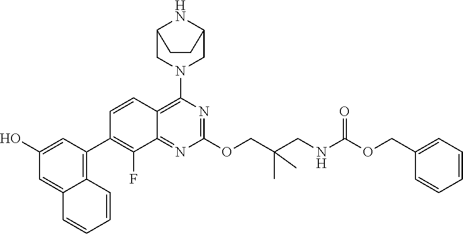 [SMILES: CC(C)(CNC(=O)OCc1ccccc1)COc1nc(N2CC3CCC(C2)N3)c2ccc(-c3cc(O)cc4ccccc34)c(F)c2n1] | benzyl (3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropyl)carbamate | 636.3 |
| P-0593 | 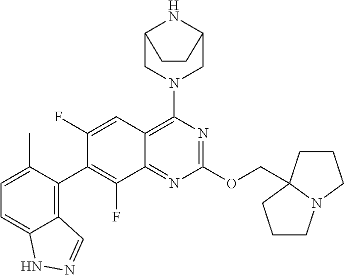 [SMILES: Cc1ccc2[nH]ncc2c1-c1c(F)cc2c(N3CC4CCC(C3)N4)nc(OCC34CCCN3CCC4)nc2c1F] | 4-((1R,5S)-3,8- diazabicyclo[3.2.1]octan-3-yl)-6,8- difluoro-7-(5-methyl-1H-indazol-4- yl)-2-((tetrahydro-1H-pyrrolizin- 7a(5H)-yl)methoxy)quinazoline | 546.2 |
| P-0594 | 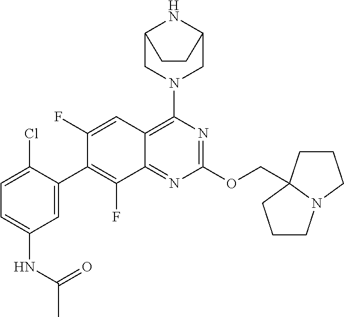 [SMILES: CC(=O)Nc1ccc(Cl)c(-c2c(F)cc3c(N4CC5CCC(C4)N5)nc(OCC45CCCN4CCC5)nc3c2F)c1] | N-(3-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 4-chlorophenyl)acetamide | 583.6 |
| P-0595 | 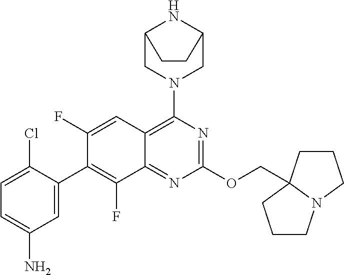 [SMILES: Nc1ccc(Cl)c(-c2c(F)cc3c(N4CC5CCC(C4)N5)nc(OCC45CCCN4CCC5)nc3c2F)c1] | 3-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 4-chloroaniline | 541.2 |
| P-0596 | 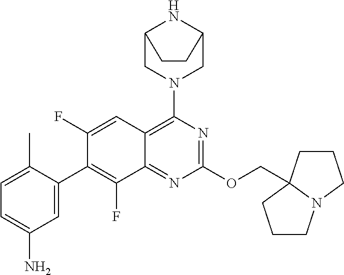 [SMILES: Cc1ccc(N)cc1-c1c(F)cc2c(N3CC4CCC(C3)N4)nc(OCC34CCCN3CCC4)nc2c1F] | 3-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 4-methylaniline | 521.3 |
| P-0598 | 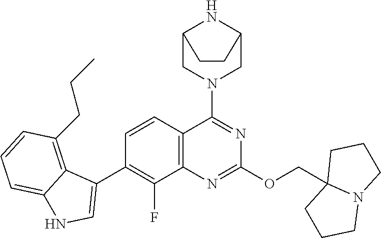 [SMILES: CCCc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(4-propyl-1H-indol-3-yl)-2- ((tetrahydro-1H-pyrrolizin-7a(5H)- yl)methoxy)quinazoline | 555.3 |
| P-0599 | 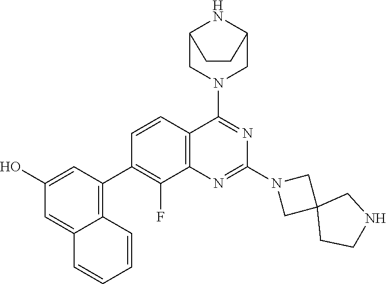 [SMILES: Oc1cc(-c2ccc3c(N4CC5CCC(C4)N5)nc(N4CC5(CCNC5)C4)nc3c2F)c2ccccc2c1] | 4-(4-((1R,5S)-3,8- diazabicyclo[3.2.1]octan-3-yl)-8- fluoro-2-(2,6-diazaspiro[3.41octan-2- yl)quinazolin-7-yl)naphthalen-2-ol | 511.3 |
| P-0600 | 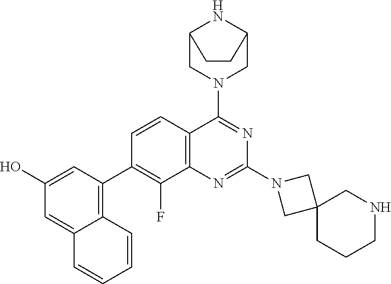 [SMILES: Oc1cc(-c2ccc3c(N4CC5CCC(C4)N5)nc(N4CC5(CCCNC5)C4)nc3c2F)c2ccccc2c1] | 4-(4-((1R,5S)-3,8- diazabicyclo[3.2.1]octan-3-yl)-8- fluoro-2-(2,6-diazaspiro[3.5]nonan-2- yl)quinazolin-7-yl)naphthalen-2-ol | 525.3 |
| P-0601 | 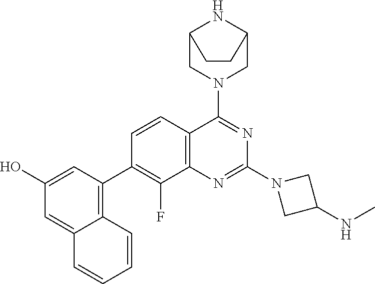 [SMILES: CNC1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3-(methylamino)azetidin-1- yl)quinazolin-7-yl)naphthalen-2-ol | 485.2 |
| P-0602 | 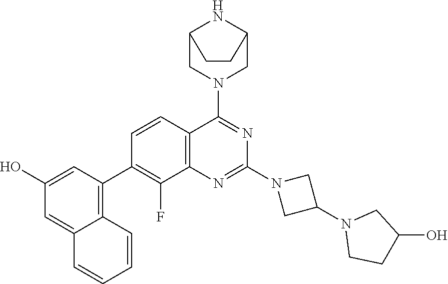 [SMILES: Oc1cc(-c2ccc3c(N4CC5CCC(C4)N5)nc(N4CC(N5CCC(O)C5)C4)nc3c2F)c2ccccc2c1] | 1-(1-(4-((1R,5S)-3,8- diazabicyclo[3.2.1]octan-3-yl)-8- fluoro-7-(3-hydroxynaphthalen-1- yl)quinazolin-2-yl)azetidin-3- yl)pyrrolidin-3-ol | 541.3 |
| P-0604 | 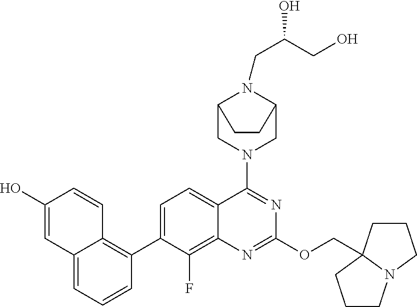 [SMILES: OC[C@@H](O)CN1C2CCC1CN(c1nc(OCC34CCCN3CCC4)nc3c(F)c(-c4cccc5cc(O)ccc45)ccc13)C2] | (S)-3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)propane-1,2-diol | 613.3 |
| P-0605 | 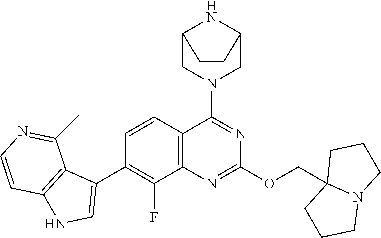 [SMILES: Cc1nccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-8- fluoro-7-(4-methyl-1H-pyrrolo[3,2- c]pyridin-3-yl)-2-((tetrahydro-1H- pyrrolizin-7a(5H)- yl)methoxy)quinazoline | 528.3 |
| P-0606 | 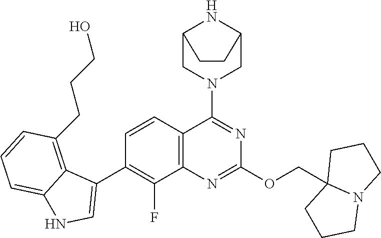 [SMILES: OCCCc1cccc2[nH]cc(-c3ccc4c(N5CC6CCC(C5)N6)nc(OCC56CCCN5CCC6)nc4c3F)c12] | 3-(3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indol-4-yl)propan-1-ol | 571.3 |
| P-0607 | 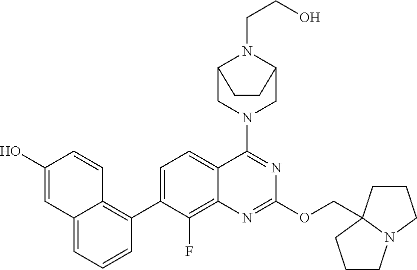 [SMILES: OCCN1C2CCC1CN(c1nc(OCC34CCCN3CCC4)nc3c(F)c(-c4cccc5cc(O)ccc45)ccc13)C2] | 4-(8-fluoro-4-((1R,5S)-8-(2- hydroxyethyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 583.3 |
| P-0608 | 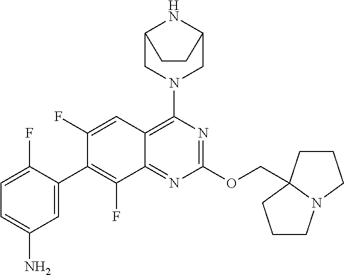 [SMILES: Nc1ccc(F)c(-c2c(F)cc3c(N4CC5CCC(C4)N5)nc(OCC45CCCN4CCC5)nc3c2F)c1] | 3-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 4-fluoroaniline | 525.2 |
| P-0609 | 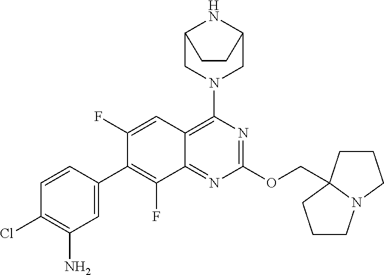 [SMILES: Nc1cc(-c2c(F)cc3c(N4CC5CCC(C4)N5)nc(OCC45CCCN4CCC5)nc3c2F)ccc1Cl] | 5-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 2-chloroaniline | 541.1 |
| P-0610 | 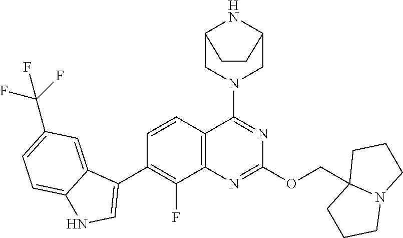 [SMILES: Fc1c(-c2c[nH]c3ccc(C(F)(F)F)cc23)ccc2c(N3CC4CCC(C3)N4)nc(OCC34CCCN3CCC4)nc12] | 4-((1R,5S)-3,8- diazabicyclo[3.2.1]octan-3-yl)-8- fluoro-2-((tetrahydro-1H-pyrrolizin- 7a(5H)-yl)methoxy)-7-(5- (trifluoromethyl)-1H-indol-3- yl)quinazoline | 581.3 |
| P-0611 | 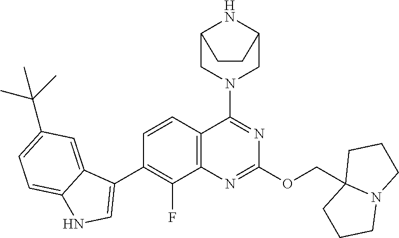 [SMILES: CC(C)(C)c1ccc2[nH]cc(-c3ccc4c(N5CC6CCC(C5)N6)nc(OCC56CCCN5CCC6)nc4c3F)c2c1] | 4-((1R,5S)-3,8- diazabicyclo[3.2.1]octan-3-yl)-7-(5- (tert-butyl)-1H-indol-3-yl)-8-fluoro-2- ((tetrahydro-1H-pyrrolizin-7a(5H)- yl)methoxy)quinazoline | 569.4 |
| P-0612 | 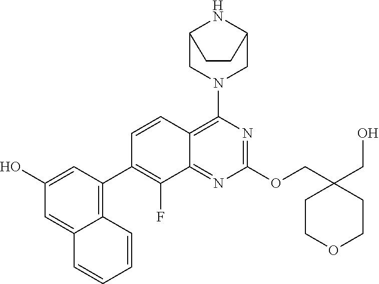 [SMILES: OCC1(COc2nc(N3CC4CCC(C3)N4)c3ccc(-c4cc(O)cc5ccccc45)c(F)c3n2)CCOCC1] | 4-(4-((1R,5S)-3,8- diazabicyclo[3.2.1]octan-3-yl)-8- fluoro-2-((4- (hydroxymethyl)tetrahydro-2H-pyran- 4-yl)methoxy)quinazolin-7- yl)naphthalen-2-ol | 643.1 |
| P-0613 | 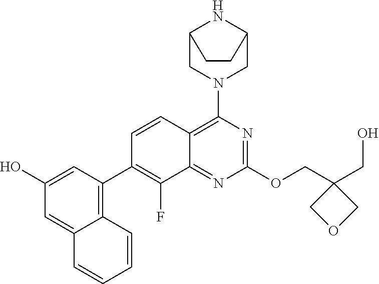 [SMILES: OCC1(COc2nc(N3CC4CCC(C3)N4)c3ccc(-c4cc(O)cc5ccccc45)c(F)c3n2)COC1] | 4-(4-((1R,5S)-3,8- diazabicyclo[3.2.1]octan-3-yl)-8- fluoro-2-((3-(hydroxymethyl)oxetan- 3-yl)methoxy)quinazolin-7- yl)naphthalen-2-ol | 515.1 |
| P-0614 | 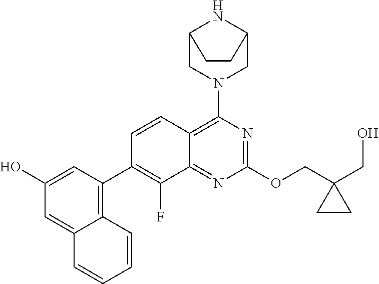 [SMILES: OCC1(COc2nc(N3CC4CCC(C3)N4)c3ccc(-c4cc(O)cc5ccccc45)c(F)c3n2)CC1] | 4-(4-((1R,5S)-3,8- diazabicyclo[3.2.1]octan-3-yl)-8- fluoro-2-((1- (hydroxymethyl)cyclopropyl)methoxy) quinazolin-7-yl)naphthalen-2-ol | 499.1 |
| P-0615 | 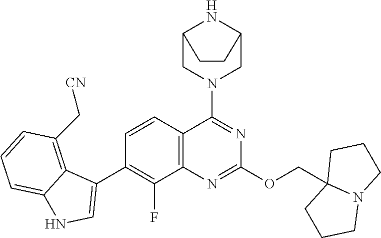 [SMILES: N#CCc1cccc2[nH]cc(-c3ccc4c(N5CC6CCC(C5)N6)nc(OCC56CCCN5CCC6)nc4c3F)c12] | 2-(3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indol-4-yl)acetonitrile | 552.2 |
| P-0616 | 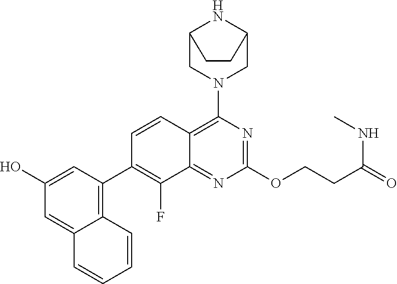 [SMILES: CNC(=O)CCOc1nc(N2CC3CCC(C2)N3)c2ccc(-c3cc(O)cc4ccccc34)c(F)c2n1] | 3-((4-((1R,5S)-3,8- diazabicyclo[3.2.1]octan-3-yl)-8- fluoro-7-(3-hydroxynaphthalen-1- yl)quinazolin-2-yl)oxy)-N- methylpropanamide | 500.1 |
| P-0617 | 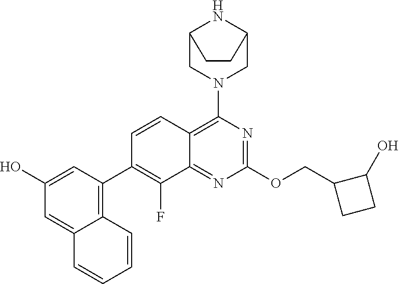 [SMILES: Oc1cc(-c2ccc3c(N4CC5CCC(C4)N5)nc(OCC4CCC4O)nc3c2F)c2ccccc2c1] | 4-(4-((1R,5S)-3,8- diazabicyclo[3.2.1]octan-3-yl)-8- fluoro-2-((2- hydroxycyclobutyl)methoxy)quinazolin- 7-yl)naphthalen-2-ol | 499.1 |
| P-0618 | 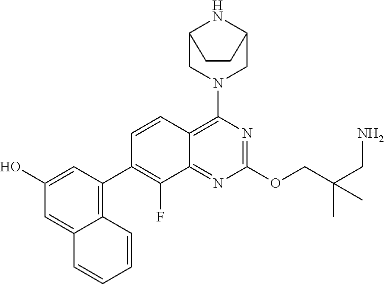 [SMILES: CC(C)(CN)COc1nc(N2CC3CCC(C2)N3)c2ccc(-c3cc(O)cc4ccccc34)c(F)c2n1] | 4-(2-(3-amino-2,2-dimethylpropoxy)- 4-((1R,5S)-3,8- diazabicyclo[3.2.1]octan-3-yl)-8- fluoroquinazolin-7-yl)naphthalen-2- ol | 502.2 |
| P-0619 | 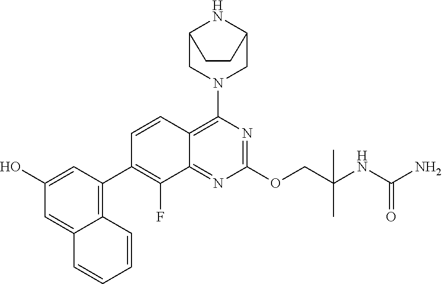 [SMILES: CC(C)(COc1nc(N2CC3CCC(C2)N3)c2ccc(-c3cc(O)cc4ccccc34)c(F)c2n1)NC(N)=O] | 1-(1-((4-((1R,5S)-3,8- diazabicyclo[3.2.1]octan-3-yl)-8- fluoro-7-(3-hydroxynaphthalen-1- yl)quinazolin-2-yl)oxy)-2- methylpropan-2-yl)urea | 531.2 |
| P-0620 | 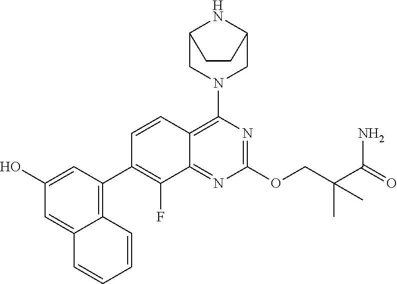 [SMILES: CC(C)(COc1nc(N2CC3CCC(C2)N3)c2ccc(-c3cc(O)cc4ccccc34)c(F)c2n1)C(N)=O] | 3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropanamide | 516.6 |
| P-0621 | 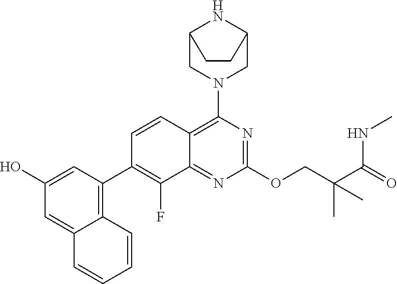 [SMILES: CNC(=O)C(C)(C)COc1nc(N2CC3CCC(C2)N3)c2ccc(-c3cc(O)cc4ccccc34)c(F)c2n1] | 3-((4-((1R,5S)-3,8- diazabicyclo[3.2.1]octan-3-yl)-8- fluoro-7-(3-hydroxynaphthalen-1- yl)quinazolin-2-yl)oxy)-N,2,2- trimethylpropanamide | 530.2 |
| P-0622 | 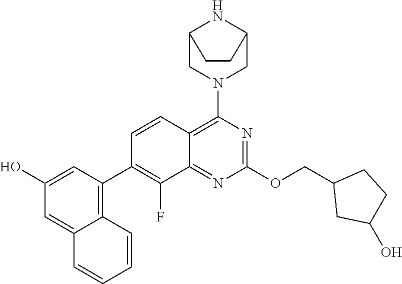 [SMILES: Oc1cc(-c2ccc3c(N4CC5CCC(C4)N5)nc(OCC4CCC(O)C4)nc3c2F)c2ccccc2c1] | 4-(4-((1R,5S)-3,8- diazabicyclo[3.2.1]octan-3-yl)-8- fluoro-2-((3- hydroxycyclopentyl)methoxy)quinazo- lin-7-yl)naphthalen-2-ol | 513.1 |
| P-0623 | 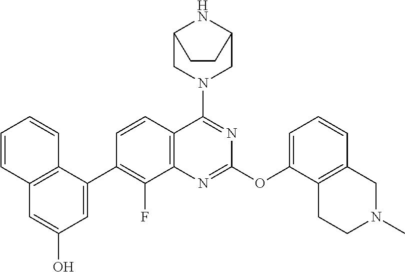 [SMILES: CN1CCc2c(cccc2Oc2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-((2-methyl-1,2,3,4- tetrahydroisoquinolin-5- yl)oxy)quinazolin-7-yl)naphthalen-2- ol | 662.3 |
| P-0624 | 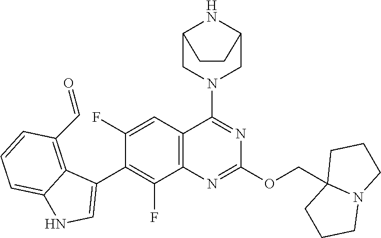 [SMILES: O=Cc1cccc2[nH]cc(-c3c(F)cc4c(N5CC6CCC(C5)N6)nc(OCC56CCCN5CCC6)nc4c3F)c12] | 3-(4-((1R,5S)-3,8- diazabicyclo[3.2.1]octan-3-yl)-6,8- difluoro-2-((tetrahydro-1H-pyrrolizin- 7a(5H)-yl)methoxy)quinazolin-7-yl)- 1H-indole-4-carbaldehyde | 559.3 |
| P-0625 | 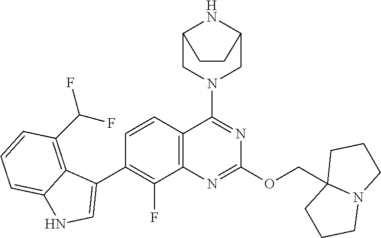 [SMILES: Fc1c(-c2c[nH]c3cccc(C(F)F)c23)ccc2c(N3CC4CCC(C3)N4)nc(OCC34CCCN3CCC4)nc12] | 4-((1R,5S)-3,8- diazabicyclo[3.2.1]octan-3-yl)-7-(4- (difluoromethyl)-1H-indol-3-yl)-8- fluoro-2-((tetrahydro-1H-pyrrolizin- 7a(5H)-yl)methoxy)quinazoline | 563.3 |
| P-0626 | 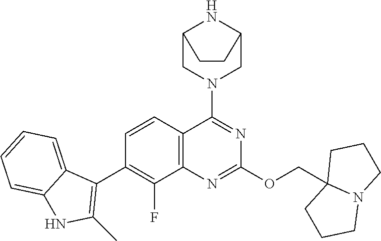 [SMILES: Cc1[nH]c2ccccc2c1-c1ccc2c(N3CC4CCC(C3)N4)nc(OCC34CCCN3CCC4)nc2c1F] | 4-((1R,5S)-3,8- diazabicyclo[3.2.1]octan-3-yl)-8- fluoro-7-(2-methyl-1H-indol-3-yl)-2- ((tetrahydro-1H-pyrrolizin-7a(5H)- yl)methoxy)quinazoline | 527.3 |
| P-0627 | 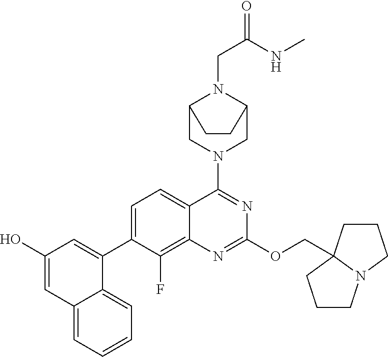 [SMILES: CNC(=O)CN1C2CCC1CN(c1nc(OCC34CCCN3CCC4)nc3c(F)c(-c4cc(O)cc5ccccc45)ccc13)C2] | 2-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-N- methylacetamide | 610.3 |
| P-0628 | 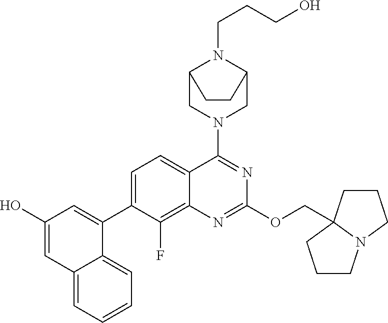 [SMILES: OCCCN1C2CCC1CN(c1nc(OCC34CCCN3CCC4)nc3c(F)c(-c4cc(O)cc5ccccc45)ccc13)C2] | 4-(8-fluoro-4-((1R,5S)-8-(3- hydroxypropyl)-3,8- diazabicyclo[3.2.1]octan-3-yl)-2- ((tetrahydro-1H-pyrrolizin-7a(5H)- yl)methoxy)quinazolin-7- yl)naphthalen-2-ol | 597.3 |
| P-0629 | 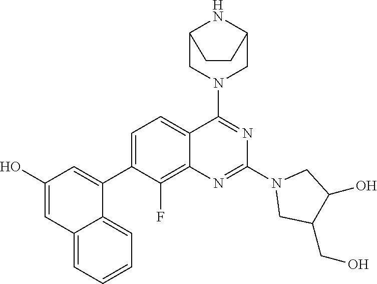 [SMILES: OCC1CN(c2nc(N3CC4CCC(C3)N4)c3ccc(-c4cc(O)cc5ccccc45)c(F)c3n2)CC1O] | 1-(4-((1R,5S)-3,8- diazabicyclo[3.2.1]octan-3-yl)-8- fluoro-7-(3-hydroxynaphthalen-1- yl)quinazolin-2-yl)-4- (hydroxymethyl)pyrrolidin-3-ol | 515.2 |
| P-0630 | 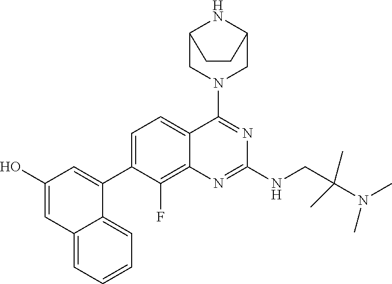 [SMILES: CN(C)C(C)(C)CNc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-((2- (dimethylamino)-2- methylpropyl)amino)-8- fluoroquinazolin-7-yl)naphthalen-2- ol | 514.3 |
| P-0631 | 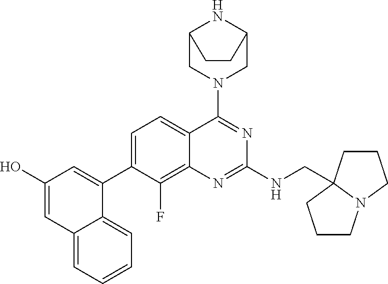 [SMILES: Oc1cc(-c2ccc3c(N4CC5CCC(C4)N5)nc(NCC45CCCN4CCC5)nc3c2F)c2ccccc2c1] | 4-(4-((1R,5S)-3,8- diazabicyclo[3.2.1]octan-3-yl)-8- fluoro-2-(((tetrahydro-1H-pyrrolizin- 7a(5H)-yl)methyl)amino)quinazolin- 7-yl)naphthalen-2-ol | 538.3 |
| P-0632 | 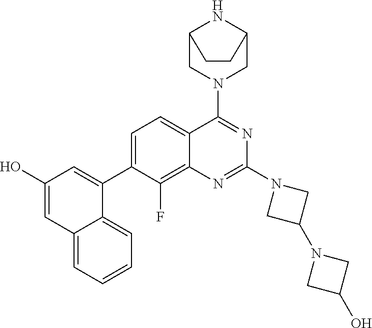 [SMILES: Oc1cc(-c2ccc3c(N4CC5CCC(C4)N5)nc(N4CC(N5CC(O)C5)C4)nc3c2F)c2ccccc2c1] | 1′-(4-((1R,5S)-3,8- diazabicyclo[3.2.1]octan-3-yl)-8- fluoro-7-(3-hydroxynaphthalen-1- yl)quinazolin-2-yl)-[1,3′-biazetidin]- 3-ol | 526.2 |
| P-0633 | 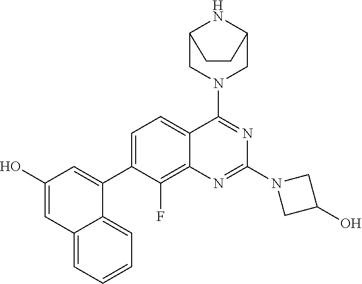 [SMILES: Oc1cc(-c2ccc3c(N4CC5CCC(C4)N5)nc(N4CC(O)C4)nc3c2F)c2ccccc2c1] | 1-(4-((1R,5S)-3,8- diazabicyclo[3.2.1]octan-3-yl)-8- fluoro-7-(3-hydroxynaphthalen-1- yl)quinazolin-2-yl)azetidin-3-ol | 471.2 |
| P-0634 | 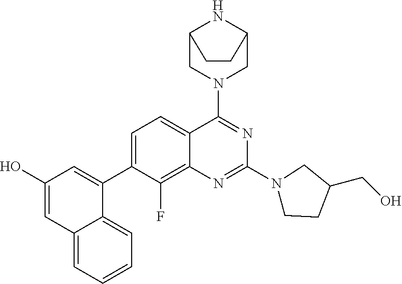 [SMILES: OCC1CCN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3- (hydroxymethyl)pyrrolidin-1- yl)quinazolin-7-yl)naphthalen-2-ol | 499.2 |
| P-0635 | 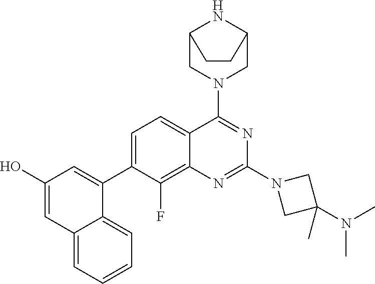 [SMILES: CN(C)C1(C)CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (dimethylamino)-3-methylazetidin-1- yl)-8-fluoroquinazolin-7- yl)naphthalen-2-ol | 512.3 |
| P-0636 | 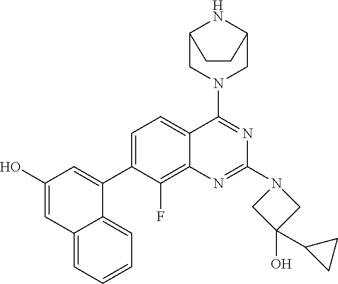 [SMILES: Oc1cc(-c2ccc3c(N4CC5CCC(C4)N5)nc(N4CC(O)(C5CC5)C4)nc3c2F)c2ccccc2c1] | 1-(4-((1R,5S)-3,8- diazabicyclo[3.2.1]octan-3-yl)-8- fluoro-7-(3-hydroxynaphthalen-1- yl)quinazolin-2-yl)-3- cyclopropylazetidin-3-ol | 511.2 |
| P-0637 | 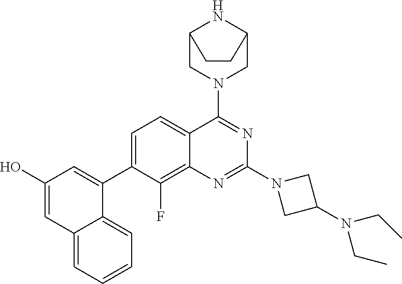 [SMILES: CCN(CC)C1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (diethylamino)azetidin-1-yl)-8- fluoroquinazolin-7-yl)naphthalen-2- ol | 526.3 |
| P-0638 | 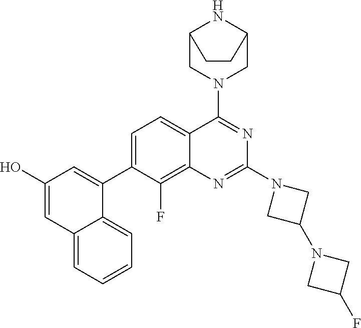 [SMILES: Oc1cc(-c2ccc3c(N4CC5CCC(C4)N5)nc(N4CC(N5CC(F)C5)C4)nc3c2F)c2ccccc2c1] | 4-(4-((1R,5S)-3,8- diazabicyclo[3.2.1]octan-3-yl)-8- fluoro-2-(3-fluoro-[1,3′-biazetidin]-1′- yl)quinazolin-7-yl)naphthalen-2-ol | 528.2 |
| P-0639 | 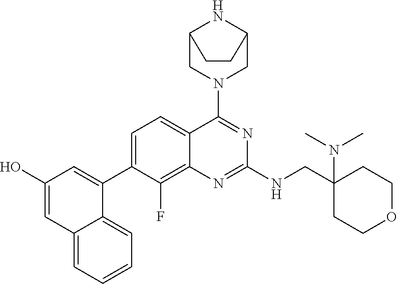 [SMILES: CN(C)C1(CNc2nc(N3CC4CCC(C3)N4)c3ccc(-c4cc(O)cc5ccccc45)c(F)c3n2)CCOCC1] | 4-(4-((1R,5S)-3,8- diazabicyclo[3.2.1]octan-3-yl)-2-(((4- (dimethylamino)tetrahydro-2H-pyran- 4-yl)methyl)amino)-8- fluoroquinazolin-7-yl)naphthalen-2- ol | 556.3 |
| P-0640 | 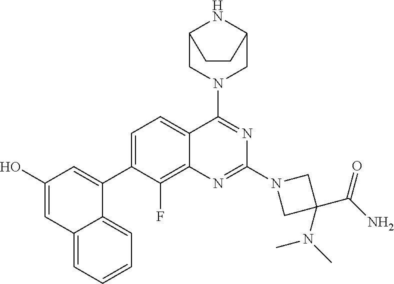 [SMILES: CN(C)C1(C(N)=O)CN(c2nc(N3CC4CCC(C3)N4)c3ccc(-c4cc(O)cc5ccccc45)c(F)c3n2)C1] | 1-(4-((1R,5S)-3,8- diazabicyclo[3.2.1]octan-3-yl)-8- fluoro-7-(3-hydroxynaphthalen-1- yl)quinazolin-2-yl)-3- (dimethylamino)azetidine-3- carboxamide | 541.3 |
| P-0641 | 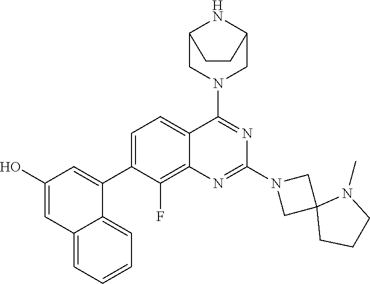 [SMILES: CN1CCCC12CN(c1nc(N3CC4CCC(C3)N4)c3ccc(-c4cc(O)cc5ccccc45)c(F)c3n1)C2] | 4-(4-((1R,5S)-3,8- diazabicyclo[3.2.1]octan-3-yl)-8- fluoro-2-(5-methyl-2,5- diazaspiro[3.4]octan-2-yl)quinazolin- 7-yl)naphthalen-2-ol | 524.3 |
| P-0642 | 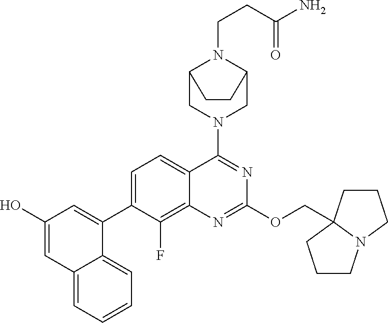 [SMILES: NC(=O)CCN1C2CCC1CN(c1nc(OCC34CCCN3CCC4)nc3c(F)c(-c4cc(O)cc5ccccc45)ccc13)C2] | 3-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8- yl)propanamide | 610.3 |
| P-0643 | 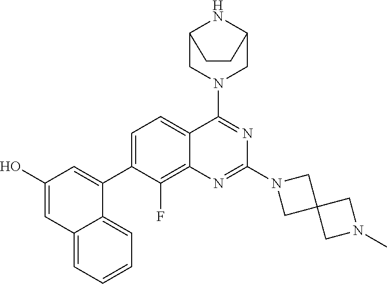 [SMILES: CN1CC2(C1)CN(c1nc(N3CC4CCC(C3)N4)c3ccc(-c4cc(O)cc5ccccc45)c(F)c3n1)C2] | 4-(4-((1R,5S)-3,8- diazabicyclo[3.2.1]octan-3-yl)-8- fluoro-2-(6-methyl-2,6- diazaspiro[3.3]heptan-2- yl)quinazolin-7-yl)naphthalen-2-ol | 510.3 |
| P-0645 | 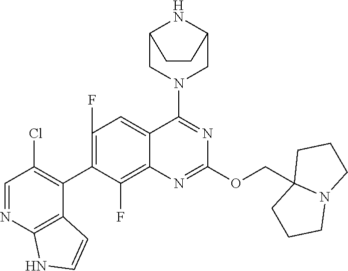 [SMILES: Fc1cc2c(N3CC4CCC(C3)N4)nc(OCC34CCCN3CCC4)nc2c(F)c1-c1c(Cl)cnc2[nH]ccc12] | 4-((1R,5S)-3,8- diazabicyclo[3.2.1]octan-3-yl)-7-(5- chloro-1H-pyrrolo[2,3-b]pyridin-4- yl)-6,8-difluoro-2-((tetrahydro-1H- pyrrolizin-7a(5H)- yl)methoxy)quinazoline | 566.2 |
| P-0646 | 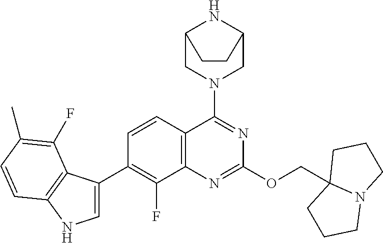 [SMILES: Cc1ccc2[nH]cc(-c3ccc4c(N5CC6CCC(C5)N6)nc(OCC56CCCN5CCC6)nc4c3F)c2c1F] | 4-((1R,5S)-3,8- diazabicyclo[3.2.1]octan-3-yl)-8- fluoro-7-(4-fluoro-5-methyl-1H-indol- 3-yl)-2-((tetrahydro-1H-pyrrolizin- 7a(5H)-yl)methoxy)quinazoline | 545.3 |
| P-0647 | 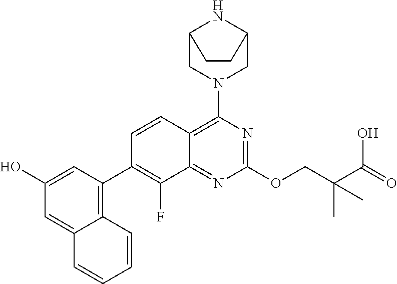 [SMILES: CC(C)(COc1nc(N2CC3CCC(C2)N3)c2ccc(-c3cc(O)cc4ccccc34)c(F)c2n1)C(=O)O] | 3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropanoic acid | 517.6 |
| P-0648 | 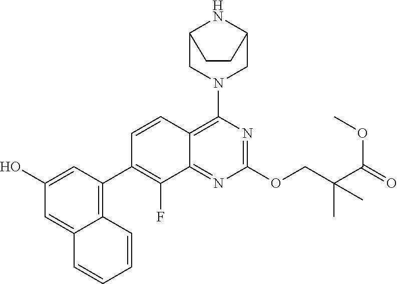 [SMILES: COC(=O)C(C)(C)COc1nc(N2CC3CCC(C2)N3)c2ccc(-c3cc(O)cc4ccccc34)c(F)c2n1] | methyl 3-((4-((1R,5S)-3,8- diazabicyclo[3.2.1]octan-3-yl)-8- fluoro-7-(3-hydroxynaphthalen-1- yl)quinazolin-2-yl)oxy)-2,2- dimethylpropanoate | 531.6 |
| P-0649 | 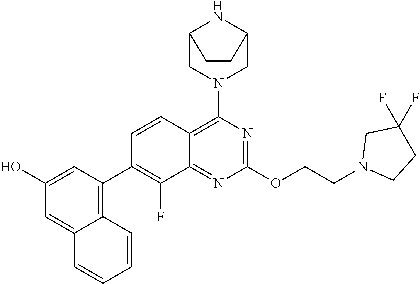 [SMILES: Oc1cc(-c2ccc3c(N4CC5CCC(C4)N5)nc(OCCN4CCC(F)(F)C4)nc3c2F)c2ccccc2c1] | 4-(4-((1R,5S)-3,8- diazabicyclo[3.2.1]octan-3-yl)-2-(2- (3,3-difluoropyrrolidin-1-yl)ethoxy)- 8-fluoroquinazolin-7-yl)naphthalen-2- ol | 548.1 |
| P-0650 | 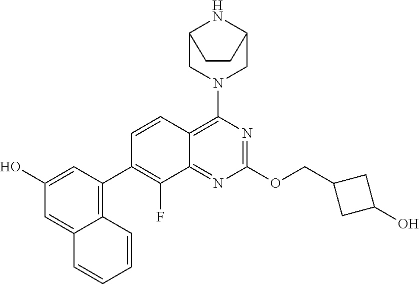 [SMILES: Oc1cc(-c2ccc3c(N4CC5CCC(C4)N5)nc(OCC4CC(O)C4)nc3c2F)c2ccccc2c1] | 4-(4-((1R,5S)-3,8- diazabicyclo[3.2.1]octan-3-yl)-8- fluoro-2-((3- hydroxycyclobutyl)methoxy)quinazolin- 7-yl)naphthalen-2-ol | 501.6 |
| P-0651 | 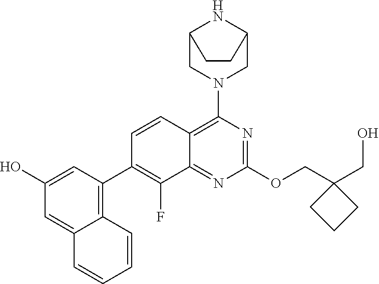 [SMILES: OCC1(COc2nc(N3CC4CCC(C3)N4)c3ccc(-c4cc(O)cc5ccccc45)c(F)c3n2)CCC1] | 4-(4-((1R,5S)-3,8- diazabicyclo[3.2.1]octan-3-yl)-8- fluoro-2-((1- (hydroxymethyl)cyclobutyl)methoxy) quinazolin-7-yl)naphthalen-2-ol | 513.1 |
| P-0652 | 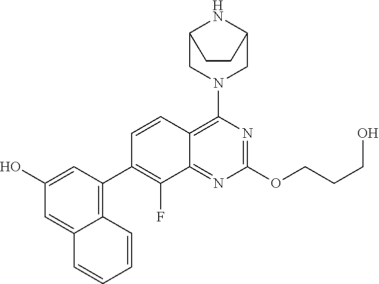 [SMILES: OCCCOc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(3- hydroxypropoxy)quinazolin-7- yl)naphthalen-2-ol | 473.1 |
| P-0653 | 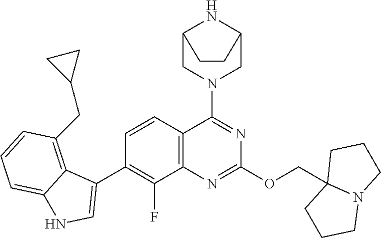 [SMILES: Fc1c(-c2c[nH]c3cccc(CC4CC4)c23)ccc2c(N3CC4CCC(C3)N4)nc(OCC34CCCN3CCC4)nc12] | 4-((1R,5S)-3,8- diazabicyclo[3.2.1]octan-3-yl)-7-(4- (cyclopropylmethyl)-1H-indol-3-yl)- 8-fluoro-2-((tetrahydro-1H-pyrrolizin- 7a(5H)-yl)methoxy)quinazoline | 567.4 |
| P-0654 | 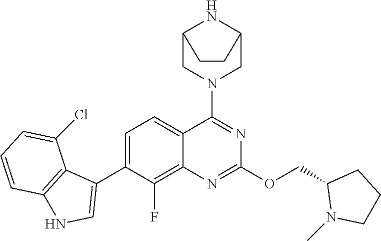 [SMILES: CN1CCC[C@H]1COc1nc(N2CC3CCC(C2)N3)c2ccc(-c3c[nH]c4cccc(Cl)c34)c(F)c2n1] | 4-((1R,5S)-3,8- diazabicyclo[3.2.1]octan-3-yl)-7-(4- chloro-1H-indol-3-yl)-8-fluoro-2- (((S)-1-methylpyrrolidin-2- yl)methoxy)quinazoline | 521.2 |
| P-0655 | 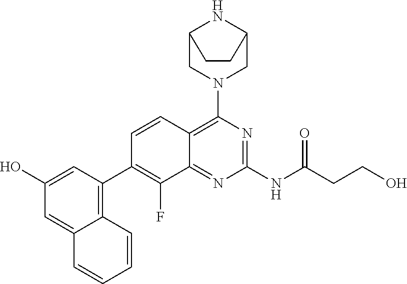 [SMILES: O=C(CCO)Nc1nc(N2CC3CCC(C2)N3)c2ccc(-c3cc(O)cc4ccccc34)c(F)c2n1] | N-(4-((1R,5S)-3,8- diazabicyclo[3.2.1]octan-3-yl)-8- fluoro-7-(3-hydroxynaphthalen-1- yl)quinazolin-2-yl)-3- hydroxypropanamide | 488.5 |
| P-0656 | 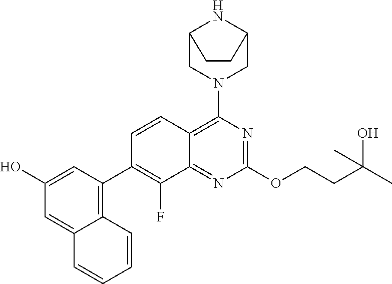 [SMILES: CC(C)(O)CCOc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(3-hydroxy-3- methylbutoxy)quinazolin-7- yl)naphthalen-2-ol | 503.6 |
| P-0658 | 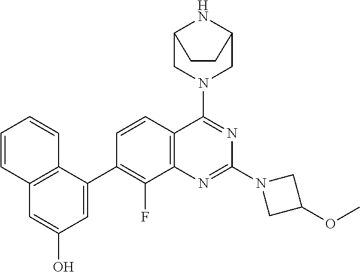 [SMILES: COC1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3-methoxyazetidin-1- yl)quinazolin-7-yl)naphthalen-2-ol | 486.2 |
| P-0659 | 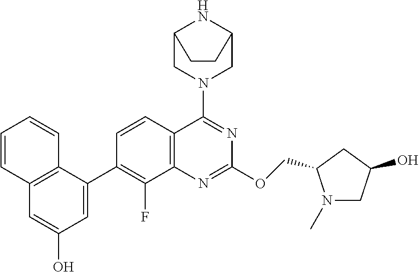 [SMILES: CN1C[C@H](O)C[C@H]1COc1nc(N2CC3CCC(C2)N3)c2ccc(-c3cc(O)cc4ccccc34)c(F)c2n1] | (3R,5S)-5-(((4-((1R,5S)-3,8- diazabicyclo[3.2.1]octan-3-yl)-8- fluoro-7-(3-hydroxynaphthalen-1- yl)quinazolin-2-yl)oxy)methyl)-1- methylpyrrolidin-3-ol | 530.3 |
| P-0660 | 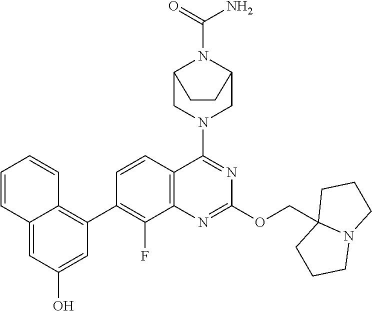 [SMILES: NC(=O)N1C2CCC1CN(c1nc(OCC34CCCN3CCC4)nc3c(F)c(-c4cc(O)cc5ccccc45)ccc13)C2] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- carboxamide | 583.3 |
| P-0661 | 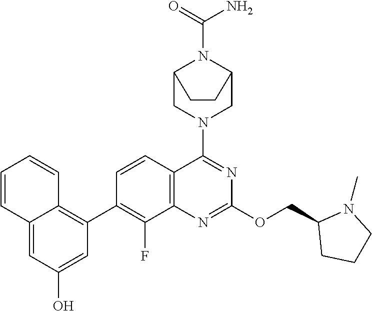 [SMILES: CN1CCC[C@H]1COc1nc(N2CC3CCC(C2)N3C(N)=O)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- carboxamide | 557.3 |
| P-0662 | 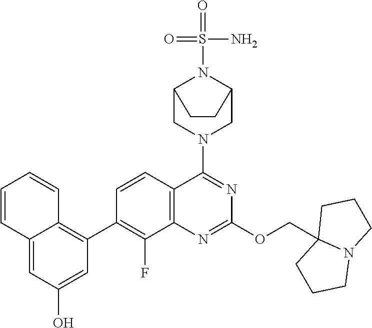 [SMILES: NS(=O)(=O)N1C2CCC1CN(c1nc(OCC34CCCN3CCC4)nc3c(F)c(-c4cc(O)cc5ccccc45)ccc13)C2] | (1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octane-8- sulfonamide | 619.3 |
| P-0663 | 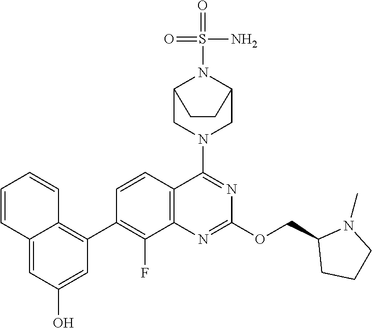 [SMILES: CN1CCC[C@H]1COc1nc(N2CC3CCC(C2)N3S(N)(=O)=O)c2ccc(-c3cc(O)cc4ccccc34)c(F)c2n1] | (1R,5S)-3-(8-fluoro-7-(3- hydroxynaphthalen-1-yl)-2-(((S)-1- methylpyrrolidin-2- yl)methoxy)quinazolin-4-yl)-3,8- diazabicyclo[3.2.1]octane-8- sulfonamide | 593.2 |
| P-0666 | 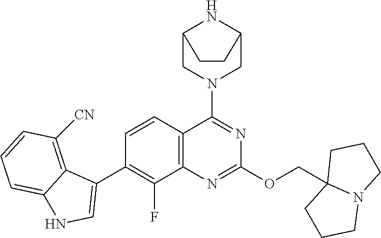 [SMILES: N#Cc1cccc2[nH]cc(-c3ccc4c(N5CC6CCC(C5)N6)nc(OCC56CCCN5CCC6)nc4c3F)c12] | 3-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 1H-indole-4-carbonitrile | 538.6 |
| P-0667 | 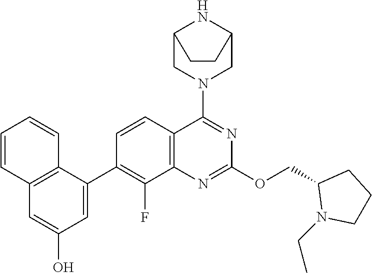 [SMILES: CCN1CCC[C@H]1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2- (((S)-1-ethylpyrrolidin-2- yl)methoxy)-8-fluoroquinazolin-7- yl)naphthalen-2-ol | 528.3 |
| P-0668 | 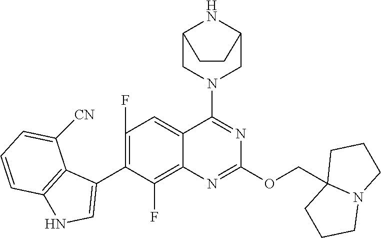 [SMILES: N#Cc1cccc2[nH]cc(-c3c(F)cc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- chloro-1H-indol-3-yl)-6,8-difluoro-2- ((tetrahydro-1H-pyrrolizin-7a(5H)- yl)methoxy)quinazoline | 565.3 |
| P-0669 | 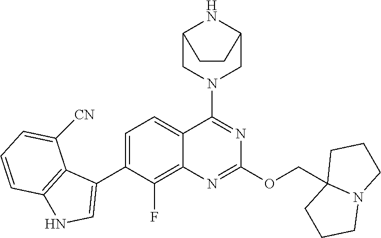 [SMILES: N#Cc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- chloro-1H-indol-3-yl)-8-fluoro-2- ((tetrahydro-1H-pyrrolizin-7a(5H)- yl)methoxy)quinazoline | 547.3 |
| P-0670 | 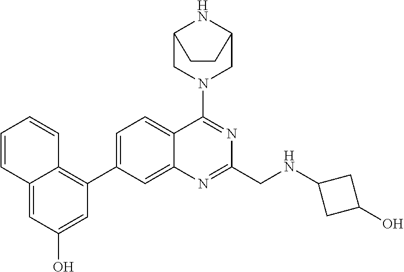 [SMILES: Oc1cc(-c2ccc3c(N4CC5CCC(C4)N5)nc(CNC4CC(O)C4)nc3c2)c2ccccc2c1] | 4-(4-((1R,5S)-3,8- diazabicyclo[3.2.1]octan-3-yl)-2-(((3- hydroxycyclobutyl)amino)methyl)quin- azolin-7-yl)naphthalen-2-ol | 482.6 |
| P-0671 | 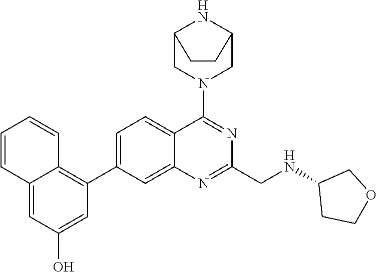 [SMILES: Oc1cc(-c2ccc3c(N4CC5CCC(C4)N5)nc(CN[C@H]4CCOC4)nc3c2)c2ccccc2c1] | 4-(4-((1R,5S)-3,8- diazabicyclo[3.2.1]octan-3-yl)-2- ((((S)-tetrahydrofuran-3- yl)amino)methyl)quinazolin-7- yl)naphthalen-2-ol | 482.6 |
| P-0672 | 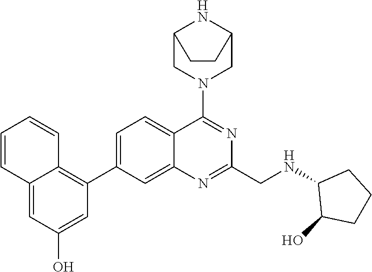 [SMILES: Oc1cc(-c2ccc3c(N4CC5CCC(C4)N5)nc(CN[C@@H]4CCC[C@H]4O)nc3c2)c2ccccc2c1] | 4-(4-((1R,5S)-3,8- diazabicyclo[3.2.1]octan-3-yl)-2- ((((1R,2R)-2- hydroxycyclopentyl)amino)methyl)quin- azolin-7-yl)naphthalen-2-ol | 496.6 |
| P-0673 | 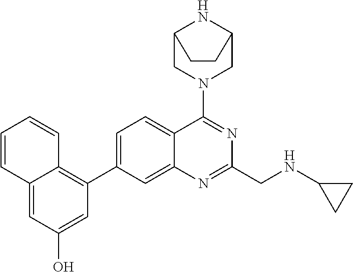 [SMILES: Oc1cc(-c2ccc3c(N4CC5CCC(C4)N5)nc(CNC4CC4)nc3c2)c2ccccc2c1] | 4-(4-((1R,5S)-3,8- diazabicyclo[3.2.1]octan-3-yl)-2- ((cyclopropylamino)methyl)quinazolin- 7-yl)naphthalen-2-ol | 452.6 |
| P-0674 | 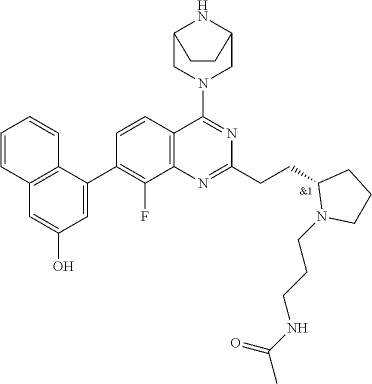 [SMILES: CC(=O)NCCCN1CCC[C@H]1CCc1nc(N2CC3CCC(C2)N3)c2ccc(-c3cc(O)cc4ccccc34)c(F)c2n1] | N-(3-((RS)-2-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-1- yl)propyl)acetamide | 599.3 |
| P-0675 | 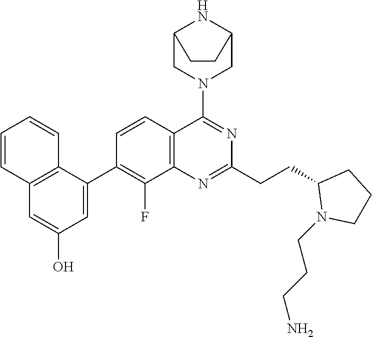 [SMILES: NCCCN1CCC[C@H]1CCc1nc(N2CC3CCC(C2)N3)c2ccc(-c3cc(O)cc4ccccc34)c(F)c2n1] | 4-(2-(((S)-1-(3- aminopropyl)pyrrolidin-2- yl)methoxy)-4-((1R,5S)-3,8- diazabicyclo[3.2.1]octan-3-yl)-8- fluoroquinazolin-7-yl)naphthalen-2- ol | 557.3 |
| P-0676 | 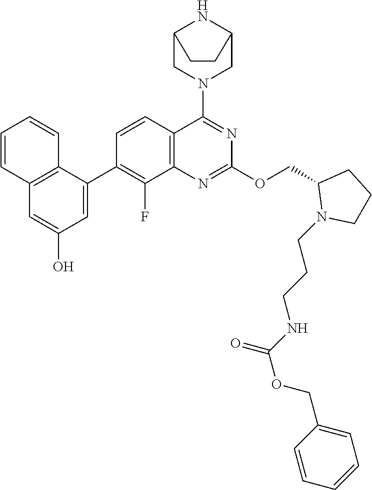 [SMILES: O=C(NCCCN1CCC[C@H]1COc1nc(N2CC3CCC(C2)N3)c2ccc(-c3cc(O)cc4ccccc34)c(F)c2n1)OCc1ccccc1] | benzyl (3-((S)-2-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-1- yl)propyl)carbamate | 691.3 |
| P-0677 | 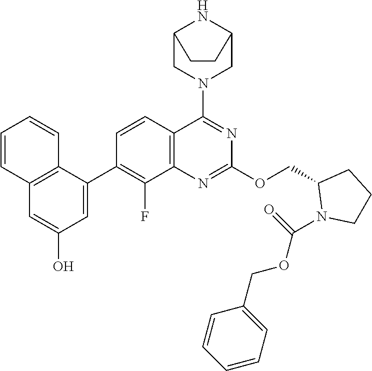 [SMILES: O=C(OCc1ccccc1)N1CCC[C@H]1COc1nc(N2CC3CCC(C2)N3)c2ccc(-c3cc(O)cc4ccccc34)c(F)c2n1] | benzyl (S)-2-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidine-1- carboxylate | 634.3 |
| P-0678 | 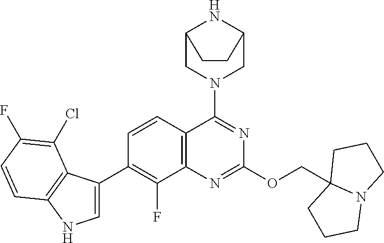 [SMILES: Fc1ccc2[nH]cc(-c3ccc4c(N5CC6CCC(C5)N6)nc(OCC56CCCN5CCC6)nc4c3F)c2c1Cl] | 4-((1R,5S)-3,8- diazabicyclo[3.2.1]octan-3-yl)-7-(4- chloro-5-fluoro-1H-indol-3-yl)-8- fluoro-2-((tetrahydro-1H-pyrrolizin- 7a(5H)-yl)methoxy)quinazoline | 565.2 |
| P-0679 | 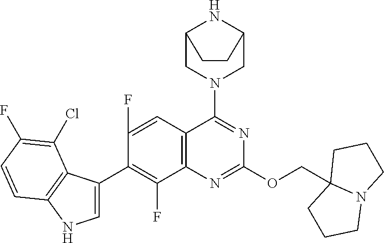 [SMILES: Fc1cc2c(N3CC4CCC(C3)N4)nc(OCC34CCCN3CCC4)nc2c(F)c1-c1c[nH]c2ccc(F)c(Cl)c12] | 4-((1R,5S)-3,8- diazabicyclo[3.2.1]octan-3-yl)-7-(4- chloro-5-fluoro-1H-indol-3-yl)-6,8- difluoro-2-((tetrahydro-1H-pyrrolizin- 7a(5H)-yl)methoxy)quinazoline | 585.2 |
| P-0680 | 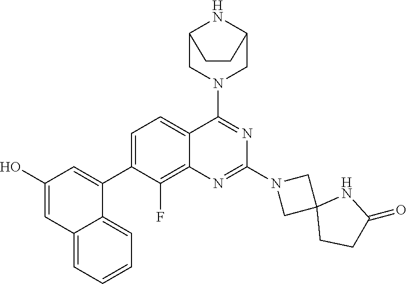 [SMILES: O=C1CCC2(CN(c3nc(N4CC5CCC(C4)N5)c4ccc(-c5cc(O)cc6ccccc56)c(F)c4n3)C2)N1] | 2-(4-((1R,5S)-3,8- diazabicyclo[3.2.1]octan-3-yl)-8- fluoro-7-(3-hydroxynaphthalen-1- yl)quinazolin-2-yl)-2,5- diazaspiro[3.4]octan-6-one | 524.2 |
| P-0681 | 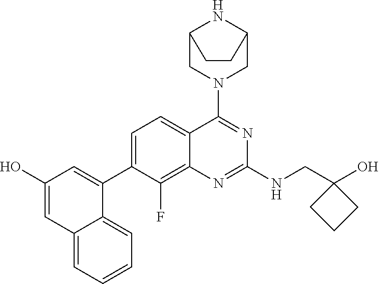 [SMILES: Oc1cc(-c2ccc3c(N4CC5CCC(C4)N5)nc(NCC4(O)CCC4)nc3c2F)c2ccccc2c1] | 4-(4-((1R,5S)-3,8- diazabicyclo[3.2.1]octan-3-yl)-8- fluoro-2-(((1- hydroxycyclobutyl)methyl)amino)quin- azolin-7-yl)naphthalen-2-ol | 499.2 |
| P-0682 | 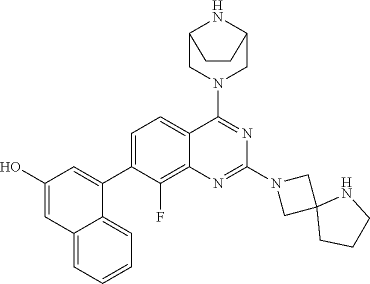 [SMILES: Oc1cc(-c2ccc3c(N4CC5CCC(C4)N5)nc(N4CC5(CCCN5)C4)nc3c2F)c2ccccc2c1] | 4-(4-((1R,5S)-3,8- diazabicyclo[3.2.1]octan-3-yl)-8- fluoro-2-(2,5-diazaspiro[3.4]octan-2- yl)quinazolin-7-yl)naphthalen-2-ol | 510.3 |
| P-0683 | 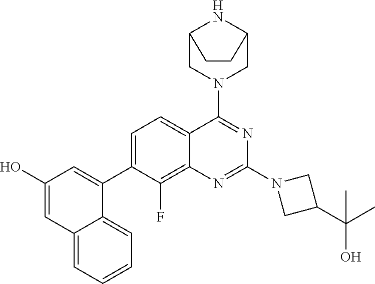 [SMILES: CC(C)(O)C1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-8- fluoro-2-(3-(2-hydroxypropan-2- yl)azetidin-1-yl)quinazolin-7- yl)naphthalen-2-ol | 513.3 |
| P-0684 | 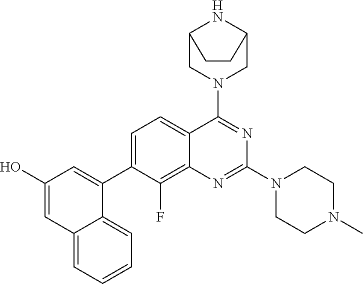 [SMILES: CN1CCN(c2nc(N3CC4CCC(C3)N4)c3ccc(-c4cc(O)cc5ccccc45)c(F)c3n2)CC1] | 4-(4-((1R,5S)-3,8- diazabicyclo[3.2.1]octan-3-yl)-8- fluoro-2-(4-methylpiperazin-1- yl)quinazolin-7-yl)naphthalen-2-ol | 498.3 |
| P-0685 | 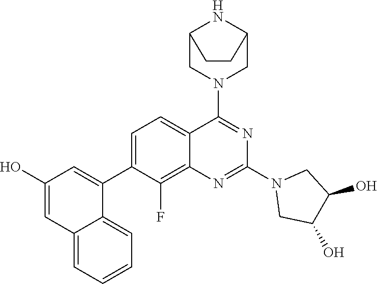 [SMILES: Oc1cc(-c2ccc3c(N4CC5CCC(C4)N5)nc(N4C[C@@H](O)[C@H](O)C4)nc3c2F)c2ccccc2c1] | (3R,4R)-1-(4-((1R,5S)-3,8- diazabicyclo[3.2.1]octan-3-yl)-8- fluoro-7-(3-hydroxynaphthalen-1- yl)quinazolin-2-yl)pyrrolidine-3,4- diol | 501.2 |
| P-0686 | 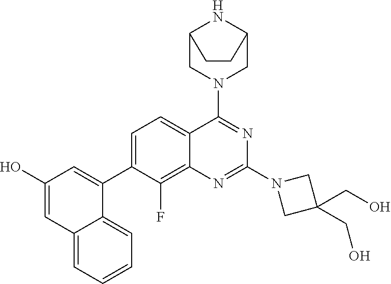 [SMILES: OCC1(CO)CN(c2nc(N3CC4CCC(C3)N4)c3ccc(-c4cc(O)cc5ccccc45)c(F)c3n2)C1] | (1-(4-((1R,5S)-3,8- diazabicyclo[3.2.1]octan-3-yl)-8- fluoro-7-(3-hydroxynaphthalen-1- yl)quinazolin-2-yl)azetidine-3,3- diyl)dimethanol | 515.2 |
| P-0687 | 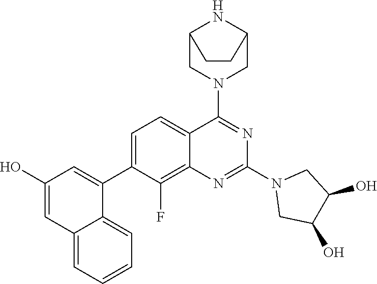 [SMILES: Oc1cc(-c2ccc3c(N4CC5CCC(C4)N5)nc(N4C[C@@H](O)[C@@H](O)C4)nc3c2F)c2ccccc2c1] | (3S,4R)-1-(4-((1R,5S)-3,8- diazabicyclo[3.2.1]octan-3-yl)-8- fluoro-7-(3-hydroxynaphthalen-1- yl)quinazolin-2-yl)pyrrolidine-3,4- diol | 501.2 |
| P-0688 | 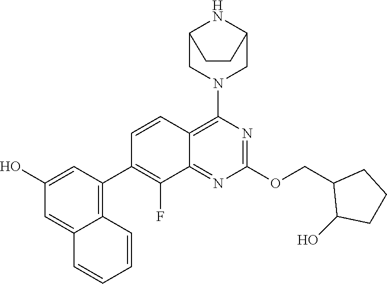 [SMILES: Oc1cc(-c2ccc3c(N4CC5CCC(C4)N5)nc(OCC4CCCC4O)nc3c2F)c2ccccc2c1] | 4-(4-((1R,5S)-3,8- diazabicyclo[3.2.1]octan-3-yl)-8- fluoro-2-((2- hydroxycyclopentyl)methoxy)quinazo- lin-7-yl)naphthalen-2-ol | 513.1 |
| P-0689 | 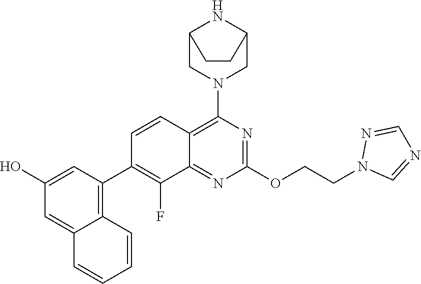 [SMILES: Oc1cc(-c2ccc3c(N4CC5CCC(C4)N5)nc(OCCn4cncn4)nc3c2F)c2ccccc2c1] | 4-(2-(2-(1H-1,2,4-triazol-1- yl)ethoxy)-4-((1R,5S)-3,8- diazabicyclo[3.2.1]octan-3-yl)-8- fluoroquinazolin-7-yl)naphthalen-2- | 510.1 |
| P-0690 | 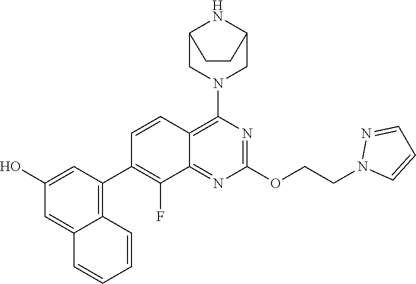 [SMILES: Oc1cc(-c2ccc3c(N4CC5CCC(C4)N5)nc(OCCn4cccn4)nc3c2F)c2ccccc2c1] | 4-(2-(2-(1H-pyrazol-1-yl)ethoxy)-4- ((1R,5S)-3,8- diazabicyclo[3.2.1]octan-3-yl)-8- fluoroquinazolin-7-yl)naphthalen-2- ol | 509.1 |
| P-0691 | 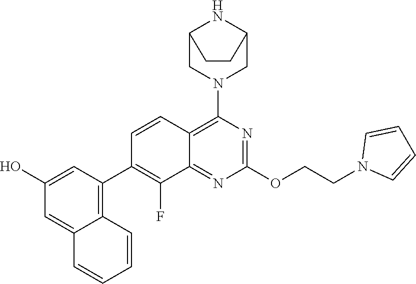 [SMILES: Oc1cc(-c2ccc3c(N4CC5CCC(C4)N5)nc(OCCn4cccc4)nc3c2F)c2ccccc2c1] | 4-(2-(2-(1H-pyrrol-1-yl)ethoxy)-4- ((1R,5S)-3,8- diazabicyclo[3.2.1]octan-3-yl)-8- fluoroquinazolin-7-yl)naphthalen-2- ol | 510.6 |
| P-0692 | 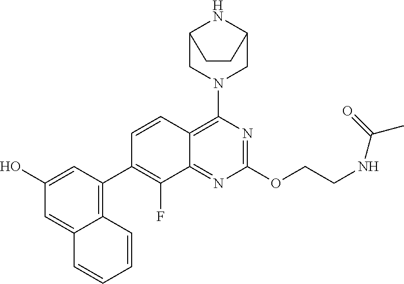 [SMILES: CC(=O)NCCOc1nc(N2CC3CCC(C2)N3)c2ccc(-c3cc(O)cc4ccccc34)c(F)c2n1] | N-(2-((4-((1R,5S)-3,8- diazabicyclo[3.2.1]octan-3-yl)-8- fluoro-7-(3-hydroxynaphthalen-1- yl)quinazolin-2- yl)oxy)ethyl)acetamide | 500.1 |
| P-0693 | 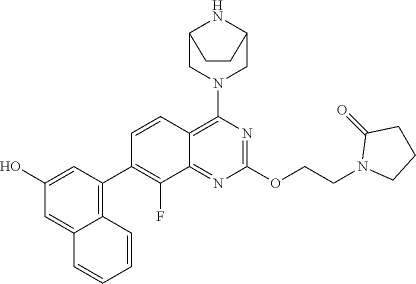 [SMILES: O=C1CCCN1CCOc1nc(N2CC3CCC(C2)N3)c2ccc(-c3cc(O)cc4ccccc34)c(F)c2n1] | 1-(2-((4-((1R,5S)-3,8- diazabicyclo[3.2.1]octan-3-yl)-8- fluoro-7-(3-hydroxynaphthalen-1- yl)quinazolin-2- yl)oxy)ethyl)pyrrolidin-2-one | 528.6 |
| P-0694 | 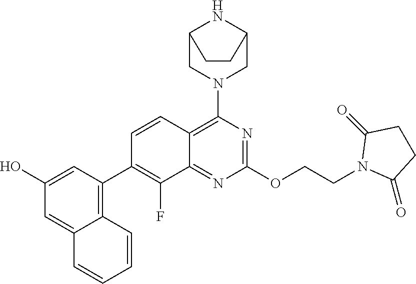 [SMILES: O=C1CCC(=O)N1CCOc1nc(N2CC3CCC(C2)N3)c2ccc(-c3cc(O)cc4ccccc34)c(F)c2n1] | 1-(2-((4-((1R,5S)-3,8- diazabicyclo[3.2.1]octan-3-yl)-8- fluoro-7-(3-hydroxynaphthalen-1- yl)quinazolin-2- yl)oxy)ethyl)pyrrolidine-2,5-dione | 542.2 |
| P-0695 | 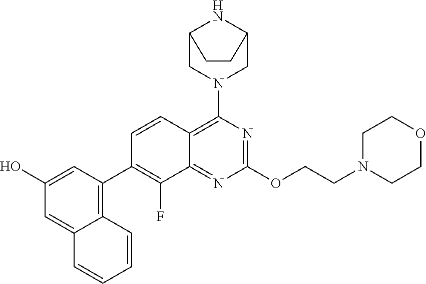 [SMILES: Oc1cc(-c2ccc3c(N4CC5CCC(C4)N5)nc(OCCN4CCOCC4)nc3c2F)c2ccccc2c1] | 4-(4-((1R,5S)-3,8- diazabicyclo[3.2.1]octan-3-yl)-8- fluoro-2-(2- morpholinoethoxy)quinazolin-7- yl)naphthalen-2-ol | 530.2 |
| P-0696 | 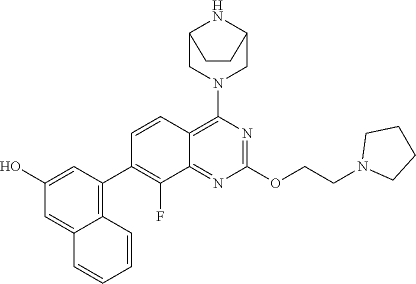 [SMILES: Oc1cc(-c2ccc3c(N4CC5CCC(C4)N5)nc(OCCN4CCCC4)nc3c2F)c2ccccc2c1] | 4-(4-((1R,5S)-3,8- diazabicyclo[3.2.1]octan-3-yl)-8- fluoro-2-(2-(pyrrolidin-1- yl)ethoxy)quinazolin-7-yl)naphthalen- 2-ol | 514.2 |
| P-0697 | 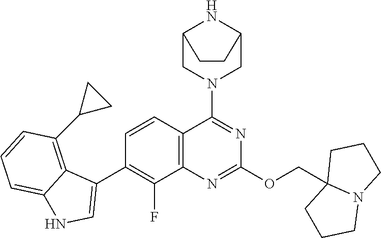 [SMILES: Fc1c(-c2c[nH]c3cccc(C4CC4)c23)ccc2c(N3CC4CCC(C3)N4)nc(OCC34CCCN3CCC4)nc12] | 4-((1R,5S)-3,8- diazabicyclo[3.2.1]octan-3-yl)-7-(4- cyclopropyl-1H-indol-3-yl)-8-fluoro- 2-((tetrahydro-1H-pyrrolizin-7a(5H)- yl)methoxy)quinazoline | 553.3 |
| P-0698 | 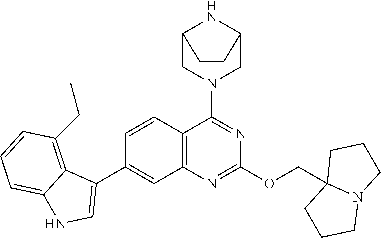 [SMILES: CCc1cccc2[nH]cc(-c3ccc4c(N5CC6CCC(C5)N6)nc(OCC56CCCN5CCC6)nc4c3)c12] | 4-((1R,5S)-3,8- diazabicyclo[3.2.1]octan-3-yl)-7-(4- ethyl-1H-indol-3-yl)-2-((tetrahydro- 1H-pyrrolizin-7a(5H)- yl)methoxy)quinazoline | 523.3 |
| P-0699 | 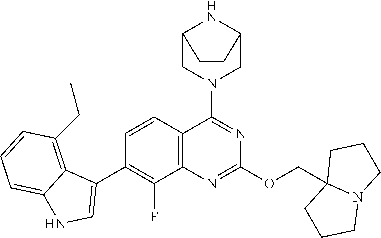 [SMILES: CCc1cccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-1H-indol-3-yl)-8-fluoro-2- ((tetrahydro-1H-pyrrolizin-7a(5H)- yl)methoxy)quinazoline | 541.3 |
| P-0700 | 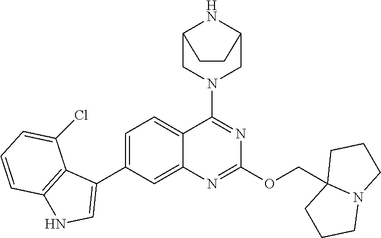 [SMILES: Clc1cccc2[nH]cc(-c3ccc4c(N5CC6CCC(C5)N6)nc(OCC56CCCN5CCC6)nc4c3)c12] | 4-((1R,5S)-3,8- diazabicyclo[3.2.1]octan-3-yl)-7-(4- chloro-1H-indol-3-yl)-2-((tetrahydro- 1H-pyrrolizin-7a(5H)- yl)methoxy)quinazoline | 529.2 |
| P-0701 | 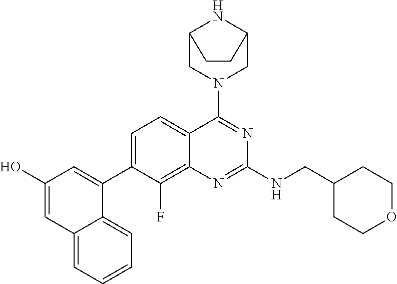 [SMILES: Oc1cc(-c2ccc3c(N4CC5CCC(C4)N5)nc(NCC4CCOCC4)nc3c2F)c2ccccc2c1] | 4-(4-((1R,5S)-3,8- diazabicyclo[3.2.1]octan-3-yl)-8- fluoro-2-(((tetrahydro-2H-pyran-4- yl)methyl)amino)quinazolin-7- yl)naphthalen-2-ol | 513.3 |
| P-0702 | 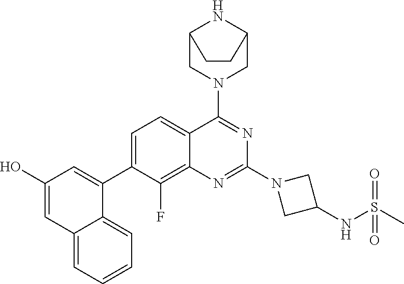 [SMILES: CS(=O)(=O)NC1CN(c2nc(N3CC4CCC(C3)N4)c3ccc(-c4cc(O)cc5ccccc45)c(F)c3n2)C1] | N-(1-(4-((1R,5S)-3,8- diazabicyclo[3.2.1]octan-3-yl)-8- fluoro-7-(3-hydroxynaphthalen-1- yl)quinazolin-2-yl)azetidin-3- yl)methanesulfonamide | 548.2 |
| P-0703 | 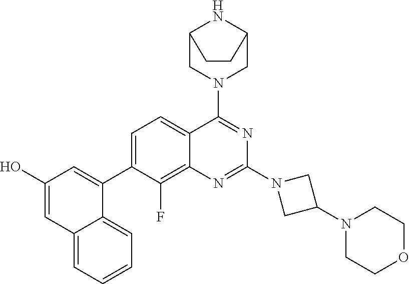 [SMILES: Oc1cc(-c2ccc3c(N4CC5CCC(C4)N5)nc(N4CC(N5CCOCC5)C4)nc3c2F)c2ccccc2c1] | 4-(4-((1R,5S)-3,8- diazabicyclo[3.2.1]octan-3-yl)-8- fluoro-2-(3-morpholinoazetidin-1- yl)quinazolin-7-yl)naphthalen-2-ol | 540.3 |
| P-0704 | 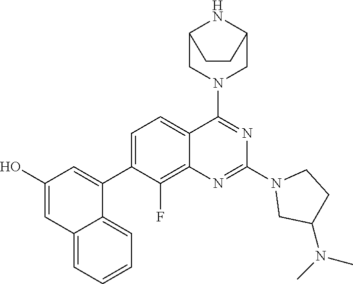 [SMILES: CN(C)C1CCN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (dimethylamino)pyrrolidin-1-yl)-8- fluoroquinazolin-7-yl)naphthalen-2- ol | 512.3 |
| P-0705 | 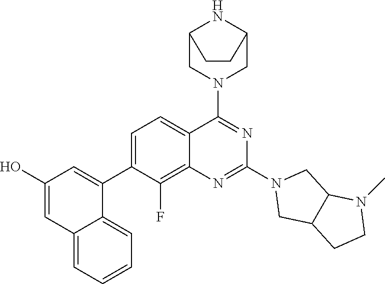 [SMILES: CN1CCC2CN(c3nc(N4CC5CCC(C4)N5)c4ccc(-c5cc(O)cc6ccccc56)c(F)c4n3)CC21] | 4-(4-((1R,5S)-3,8- diazabicyclo[3.2.1]octan-3-yl)-8- fluoro-2-(1- methylhexahydropyrrolo[3,4-b]pyrrol- 5(1H)-yl)quinazolin-7-yl)naphthalen- 2-ol | 524.3 |
| P-0706 | 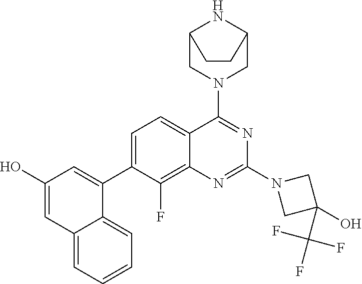 [SMILES: Oc1cc(-c2ccc3c(N4CC5CCC(C4)N5)nc(N4CC(O)(C(F)(F)F)C4)nc3c2F)c2ccccc2c1] | 1-(4-((1R,5S)-3,8- diazabicyclo[3.2.1]octan-3-yl)-8- fluoro-7-(3-hydroxynaphthalen-1- yl)quinazolin-2-yl)-3- (trifluoromethyl)azetidin-3-ol | 539.2 |
| P-0709 | 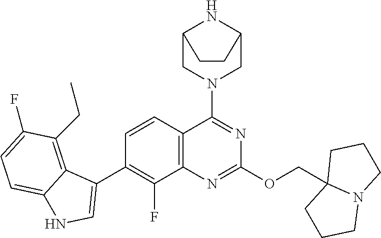 [SMILES: CCc1c(F)ccc2[nH]cc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-5-fluoro-1H-indol-3-yl)-8- fluoro-2-((tetrahydro-1H-pyrrolizin- 7a(5H)-yl)methoxy)quinazoline | 559.3 |
| P-0710 | 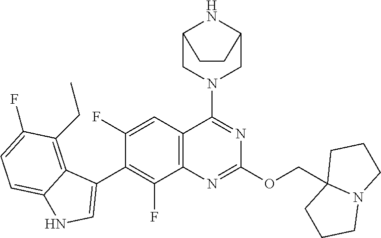 [SMILES: CCc1c(F)ccc2[nH]cc(-c3c(F)cc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-5-fluoro-1H-indol-3-yl)-6,8- difluoro-2-((tetrahydro-1H-pyrrolizin- 7a(5H)-yl)methoxy)quinazoline | 577.7 |
| P-0711 | 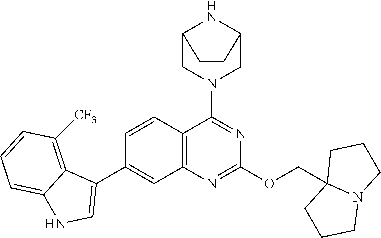 [SMILES: FC(F)(F)c1cccc2[nH]cc(-c3ccc4c(N5CC6CCC(C5)N6)nc(OCC56CCCN5CCC6)nc4c3)c12] | 4-((1R,5S)-3,8- diazabicyclo[3.2.1]octan-3-yl)-2- ((tetrahydro-1H-pyrrolizin-7a(5H)- yl)methoxy)-7-(4-(trifluoromethyl)- 1H-indol-3-yl)quinazoline | 563.3 |
| P-0712 | 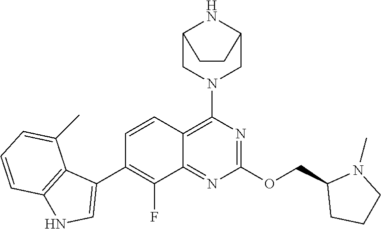 [SMILES: Cc1cccc2[nH]cc(-c3ccc4c(N5CC6CCC(C5)N6)nc(OC[C@@H]5CCCN5C)nc4c3F)c12] | 4-((1R,5S)-3,8- diazabicyclo[3.2.1]octan-3-yl)-8- fluoro-7-(4-methyl-1H-indazol-3-yl)- 2-(((S)-1-methylpyrrolidin-2- yl)methoxy)quinazoline | 502.2 |
| P-0713 | 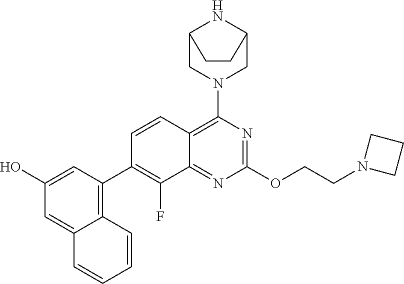 [SMILES: Oc1cc(-c2ccc3c(N4CC5CCC(C4)N5)nc(OCCN4CCC4)nc3c2F)c2ccccc2c1] | 4-(2-(2-(azetidin-1-yl)ethoxy)-4- ((1R,5S)-3,8- diazabicyclo[3.2.1]octan-3-yl)-8- fluoroquinazolin-7-yl)naphthalen-2- ol | 500.2 |
| P-0714 | 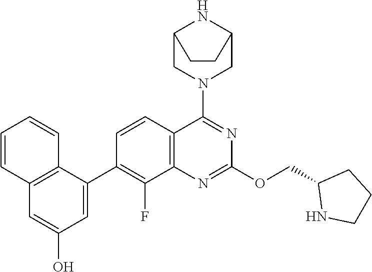 [SMILES: Oc1cc(-c2ccc3c(N4CC5CCC(C4)N5)nc(OC[C@@H]4CCCN4)nc3c2F)c2ccccc2c1] | 4-(4-((1R,5S)-3,8- diazabicyclo[3.2.1]octan-3-yl)-8- fluoro-2-(((S)-pyrrolidin-2- yl)methoxy)quinazolin-7- yl)naphthalen-2-ol | 500.2 |
| P-0715 | 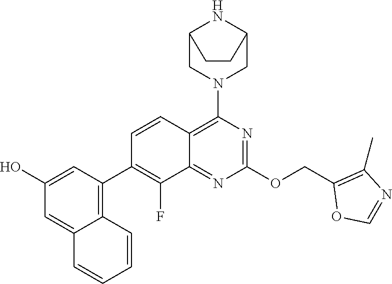 [SMILES: Cc1ncoc1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-((4-methyloxazol-5- yl)methoxy)quinazolin-7- yl)naphthalen-2-ol | 510.1 |
| P-0716 | 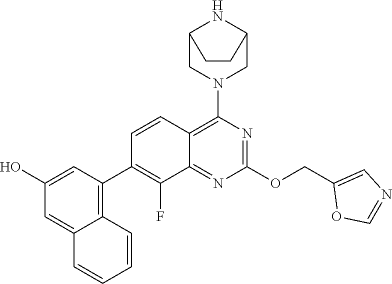 [SMILES: Oc1cc(-c2ccc3c(N4CC5CCC(C4)N5)nc(OCc4cnco4)nc3c2F)c2ccccc2c1] | 4-(4-((1R,5S)-3,8- diazabicyclo[3.2.1]octan-3-yl)-8- fluoro-2-(oxazol-5- ylmethoxy)quinazolin-7- yl)naphthalen-2-ol | 498.2 |
| P-0717 | 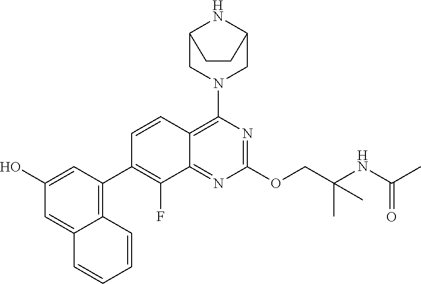 [SMILES: CC(=O)NC(C)(C)COc1nc(N2CC3CCC(C2)N3)c2ccc(-c3cc(O)cc4ccccc34)c(F)c2n1] | N-(1-((4-((1R,5S)-3,8- diazabicyclo[3.2.1]octan-3-yl)-8- fluoro-7-(3-hydroxynaphthalen-1- yl)quinazolin-2-yl)oxy)-2- methylpropan-2-yl)acetamide | 530.5 |
| P-0718 | 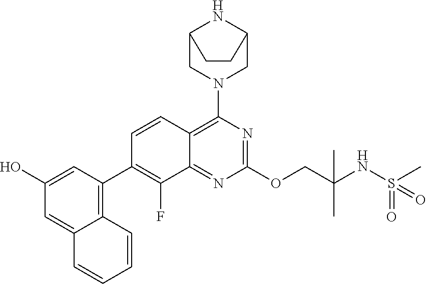 [SMILES: CC(C)(COc1nc(N2CC3CCC(C2)N3)c2ccc(-c3cc(O)cc4ccccc34)c(F)c2n1)NS(C)(=O)=O] | N-(1-((4-((1R,5S)-3,8- diazabicyclo[3.2.1]octan-3-yl)-8- fluoro-7-(3-hydroxynaphthalen-1- yl)quinazolin-2-yl)oxy)-2- methylpropan-2- yl)methanesulfonamide | 566.2 |
| P-0719 | 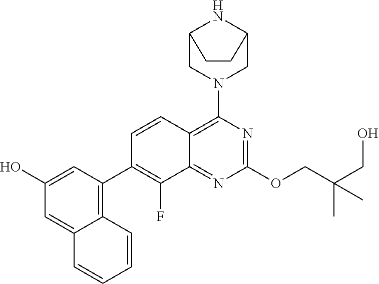 [SMILES: CC(C)(CO)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(3-hydroxy-2,2- dimethylpropoxy)quinazolin-7- yl)naphthalen-2-ol | 503.6 |
| P-0720 | 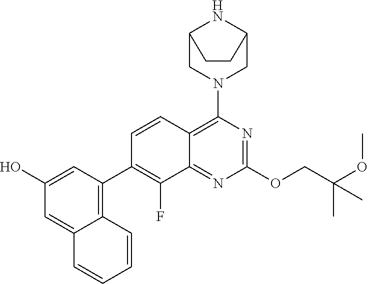 [SMILES: COC(C)(C)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2-methoxy-2- methylpropoxy)quinazolin-7- yl)naphthalen-2-ol | 503.6 |
| P-0721 | 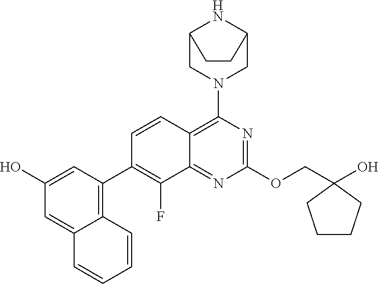 [SMILES: Oc1cc(-c2ccc3c(N4CC5CCC(C4)N5)nc(OCC4(O)CCCC4)nc3c2F)c2ccccc2c1] | 4-(4-((1R,5S)-3,8- diazabicyclo[3.2.1]octan-3-yl)-8- fluoro-2-((1- hydroxycyclopentyl)methoxy)quinazo- lin-7-yl)naphthalen-2-ol | 515.2 |
| P-0722 | 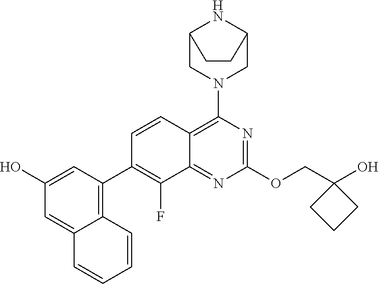 [SMILES: Oc1cc(-c2ccc3c(N4CC5CCC(C4)N5)nc(OCC4(O)CCC4)nc3c2F)c2ccccc2c1] | 4-(4-((1R,5S)-3,8- diazabicyclo[3.2.1]octan-3-yl)-8- fluoro-2-((1- hydroxycyclobutyl)methoxy)quinazolin- 7-yl)naphthalen-2-ol | 499.1 |
| P-0723 | 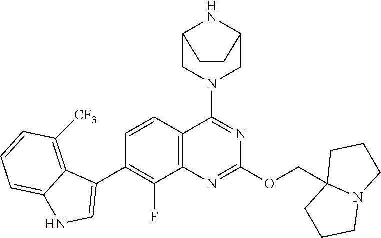 [SMILES: Fc1c(-c2c[nH]c3cccc(C(F)(F)F)c23)ccc2c(N3CC4CCC(C3)N4)nc(OCC34CCCN3CCC4)nc12] | 4-((1R,5S)-3,8- diazabicyclo[3.2.1]octan-3-yl)-8- fluoro-2-((tetrahydro-1H-pyrrolizin- 7a(5H)-yl)methoxy)-7-(4- (trifluoromethyl)-1H-indol-3- yl)quinazoline | 581.6 |
| P-0724 | 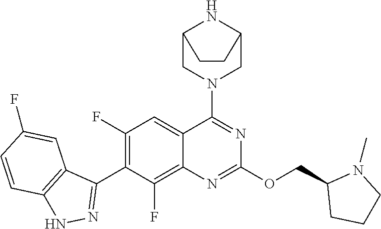 [SMILES: CN1CCC[C@H]1COc1nc(N2CC3CCC(C2)N3)c2cc(F)c(-c3n[nH]c4ccc(F)cc34)c(F)c2n1] | 4-((1R,5S)-3,8- diazabicyclo[3.2.1]octan-3-yl)-6,8- difluoro-7-(5-fluoro-1H-indazol-3-yl)- 2-(((S)-1-methylpyrrolidin-2- yl)methoxy)quinazoline | 524.5 |
| P-0725 | 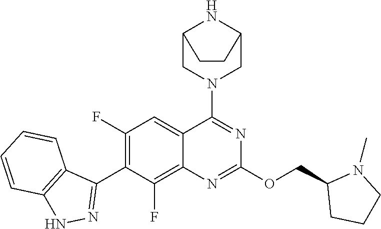 [SMILES: CN1CCC[C@H]1COc1nc(N2CC3CCC(C2)N3)c2cc(F)c(-c3n[nH]c4ccccc34)c(F)c2n1] | 4-((1R,5S)-3,8- diazabicyclo[3.2.1]octan-3-yl)-6,8- difluoro-7-(1H-indazol-3-yl)-2-(((S)- 1-methylpyrrolidin-2- yl)methoxy)quinazoline | 506.2 |
| P-0726 | 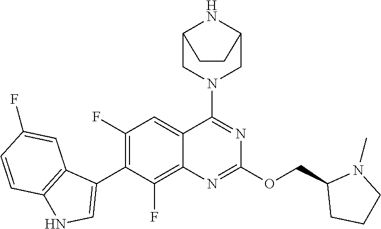 [SMILES: CN1CCC[C@H]1COc1nc(N2CC3CCC(C2)N3)c2cc(F)c(-c3c[nH]c4ccc(F)cc34)c(F)c2n1] | 4-((1R,5S)-3,8- diazabicyclo[3.2.1]octan-3-yl)-6,8- difluoro-7-(5-fluoro-1H-indol-3-yl)-2- (((S)-1-methylpyrrolidin-2- yl)methoxy)quinazoline | 523.6 |
| P-0727 | 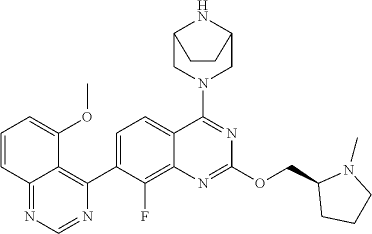 [SMILES: COc1cccc2ncnc(-c3ccc4c(N5CC6CCC(C5)N6)nc(OC[C@@H]5CCCN5C)nc4c3F)c12] | 4′-((1R,5S)-3,8- diazabicyclo[3.2.1]octan-3-yl)-8′- fluoro-5-methoxy-2′-(((S)-1- methylpyrrolidin-2-yl)methoxy)-4,7′- biquinazoline | 530.5 |
| P-0728 | 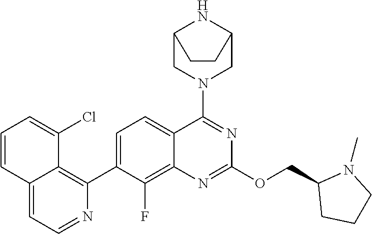 [SMILES: CN1CCC[C@H]1COc1nc(N2CC3CCC(C2)N3)c2ccc(-c3nccc4cccc(Cl)c34)c(F)c2n1] | 4-((1R,5S)-3,8- diazabicyclo[3.2.1]octan-3-yl)-7-(8- chloroisoquinolin-1-yl)-8-fluoro-2- (((S)-1-methylpyrrolidin-2- yl)methoxy)quinazoline | 534.7 |
| P-0729 | 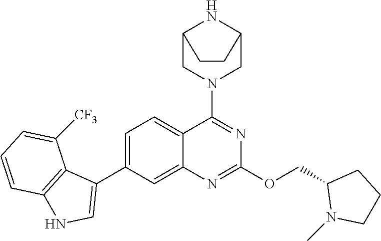 [SMILES: CN1CCC[C@H]1COc1nc(N2CC3CCC(C2)N3)c2ccc(-c3c[nH]c4cccc(C(F)(F)F)c34)cc2n1] | 4-((1R,5S)-3,8- diazabicyclo[3.2.1]octan-3-yl)-2- (((S)-1-methylpyrrolidin-2- yl)methoxy)-7-(4-(trifluoromethyl)- 1H-indol-3-yl)quinazoline | 537.2 |
| P-0730 | 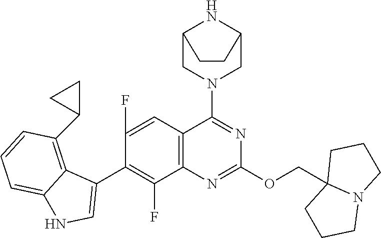 [SMILES: Fc1cc2c(N3CC4CCC(C3)N4)nc(OCC34CCCN3CCC4)nc2c(F)c1-c1c[nH]c2cccc(C3CC3)c12] | 4-((1R,5S)-3,8- diazabicyclo[3.2.1]octan-3-yl)-7-(4- cyclopropyl-1H-indol-3-yl)-6,8- difluoro-2-((tetrahydro-1H-pyrrolizin- 7a(5H)-yl)methoxy)quinazoline | 571.3 |
| P-0731 | 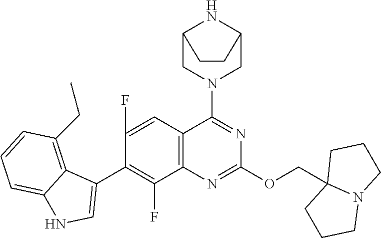 [SMILES: CCc1cccc2[nH]cc(-c3c(F)cc4c(N5CC6CCC(C5)N6)nc(OCC56CCCN5CCC6)nc4c3F)c12] | 4-((1R,5S)-3,8- diazabicyclo[3.2.1]octan-3-yl)-7-(4- ethyl-1H-indol-3-yl)-6,8-difluoro-2- ((tetrahydro-1H-pyrrolizin-7a(5H)- yl)methoxy)quinazoline | 559.3 |
| P-0732 | 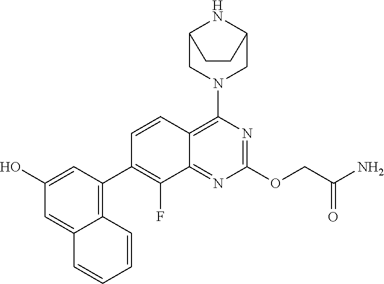 [SMILES: NC(=O)COc1nc(N2CC3CCC(C2)N3)c2ccc(-c3cc(O)cc4ccccc34)c(F)c2n1] | 2-((4-((1R,5S)-3,8- diazabicyclo[3.2.1]octan-3-yl)-8- fluoro-7-(3-hydroxynaphthalen-1- yl)quinazolin-2-yl)oxy)acetamide | 474.2 |
| P-0733 | 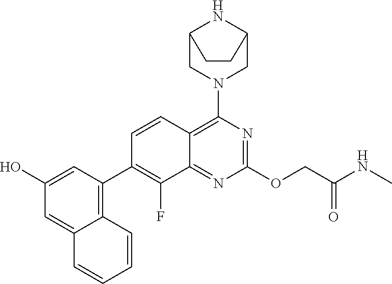 [SMILES: CNC(=O)COc1nc(N2CC3CCC(C2)N3)c2ccc(-c3cc(O)cc4ccccc34)c(F)c2n1] | 2-((4-((1R,5S)-3,8- diazabicyclo[3.2.1]octan-3-yl)-8- fluoro-7-(3-hydroxynaphthalen-1- yl)quinazolin-2-yl)oxy)-N- methylacetamide | 488.1 |
| P-0734 | 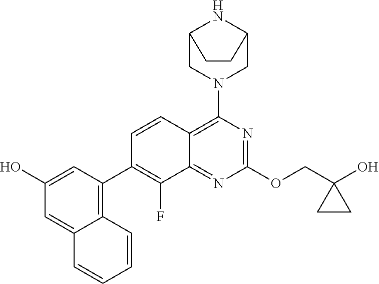 [SMILES: Oc1cc(-c2ccc3c(N4CC5CCC(C4)N5)nc(OCC4(O)CC4)nc3c2F)c2ccccc2c1] | 4-(4-((1R,5S)-3,8- diazabicyclo[3.2.1]octan-3-yl)-8- fluoro-2-((1- hydroxycyclopropyl)methoxy)quinazo- lin-7-yl)naphthalen-2-ol | 487.2 |
| P-0735 | 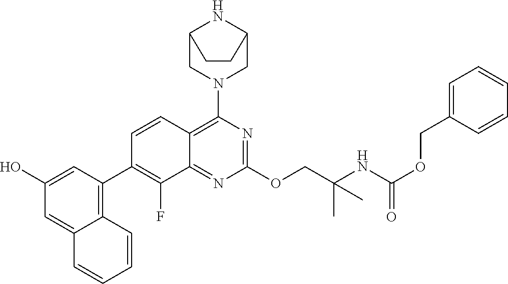 [SMILES: CC(C)(COc1nc(N2CC3CCC(C2)N3)c2ccc(-c3cc(O)cc4ccccc34)c(F)c2n1)NC(=O)OCc1ccccc1] | benzyl (1-((4-((1R,5S)-3,8- diazabicyclo[3.2.1]octan-3-yl)-8- fluoro-7-(3-hydroxynaphthalen-1- yl)quinazolin-2-yl)oxy)-2- methylpropan-2-yl)carbamate | 622.5 |
| P-0736 | 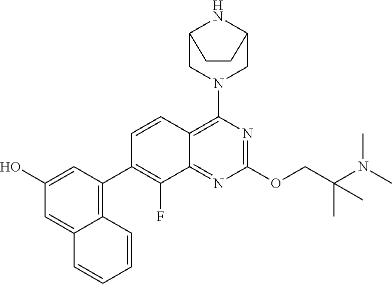 [SMILES: CN(C)C(C)(C)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-(2- (dimethylamino)-2-methylpropoxy)- 8-fluoroquinazolin-7-yl)naphthalen-2- ol | 516.6 |
| P-0737 | 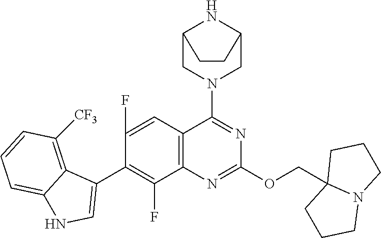 [SMILES: Fc1cc2c(N3CC4CCC(C3)N4)nc(OCC34CCCN3CCC4)nc2c(F)c1-c1c[nH]c2cccc(C(F)(F)F)c12] | 4-((1R,5S)-3,8- diazabicyclo[3.2.1]octan-3-yl)-6,8- difluoro-2-((tetrahydro-1H-pyrrolizin- 7a(5H)-yl)methoxy)-7-(4- (trifluoromethyl)-1H-indol-3- yl)quinazoline | 599.5 |
| P-0738 | 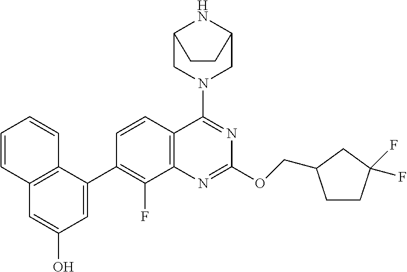 [SMILES: Oc1cc(-c2ccc3c(N4CC5CCC(C4)N5)nc(OCC4CCC(F)(F)C4)nc3c2F)c2ccccc2c1] | 4-(4-((1R,5S)-3,8- diazabicyclo[3.2.1]octan-3-yl)-2- ((3,3-difluorocyclopentyl)methoxy)-8- fluoroquinazolin-7-yl)naphthalen-2- ol | 535.3 |
| P-0739 | 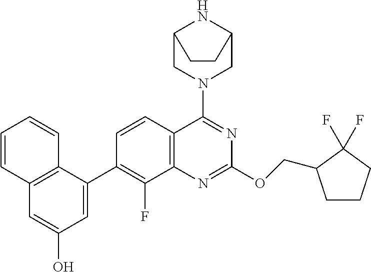 [SMILES: Oc1cc(-c2ccc3c(N4CC5CCC(C4)N5)nc(OCC4CCCC4(F)F)nc3c2F)c2ccccc2c1] | 4-(4-((1R,5S)-3,8- diazabicyclo[3.2.1]octan-3-yl)-2- ((2,2-difluorocyclopentyl)methoxy)-8- fluoroquinazolin-7-yl)naphthalen-2- ol | 535.3 |
| P-0740 | 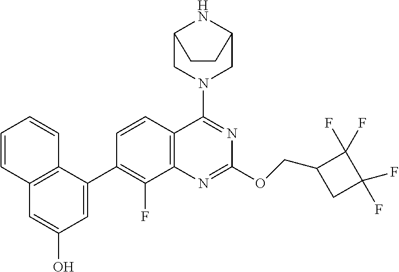 [SMILES: Oc1cc(-c2ccc3c(N4CC5CCC(C4)N5)nc(OCC4CC(F)(F)C4(F)F)nc3c2F)c2ccccc2c1] | 4-(4-((1R,5S)-3,8- diazabicyclo[3.2.1]octan-3-yl)-8- fluoro-2-((2,2,3,3- tetrafluorocyclobutyl)methoxy)quinaz- olin-7-yl)naphthalen-2-ol | 557.2 |
| P-0741 | 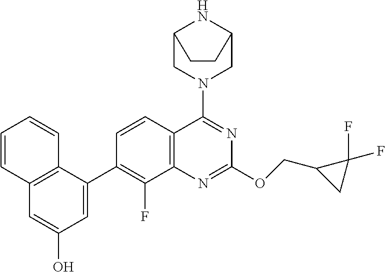 [SMILES: Oc1cc(-c2ccc3c(N4CC5CCC(C4)N5)nc(OCC4CC4(F)F)nc3c2F)c2ccccc2c1] | 4-(4-((1R,5S)-3,8- diazabicyclo[3.2.1]octan-3-yl)-2- ((2,2-difluorocyclopropyl)methoxy)- 8-fluoroquinazolin-7-yl)naphthalen-2- ol | 507.2 |
| P-0742 | 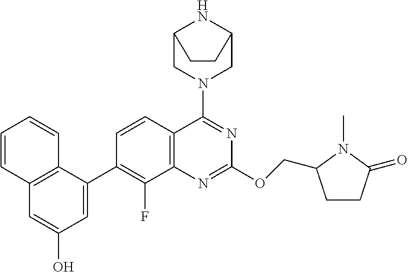 [SMILES: CN1C(=O)CCC1COc1nc(N2CC3CCC(C2)N3)c2ccc(-c3cc(O)cc4ccccc34)c(F)c2n1] | 5-(((4-((1R,5S)-3,8- diazabicyclo[3.2.1]octan-3-yl)-8- fluoro-7-(3-hydroxynaphthalen-1- yl)quinazolin-2-yl)oxy)methyl)-1- methylpyrrolidin-2-one | 528.3 |
| P-0743 | 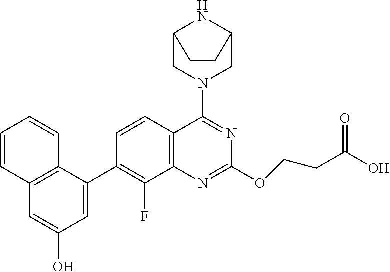 [SMILES: O=C(O)CCOc1nc(N2CC3CCC(C2)N3)c2ccc(-c3cc(O)cc4ccccc34)c(F)c2n1] | 3-((4-((1R,5S)-3,8- diazabicyclo[3.2.1]octan-3-yl)-8- fluoro-7-(3-hydroxynaphthalen-1- yl)quinazolin-2-yl)oxy)propanoic acid | 489.2 |
| P-0744 | 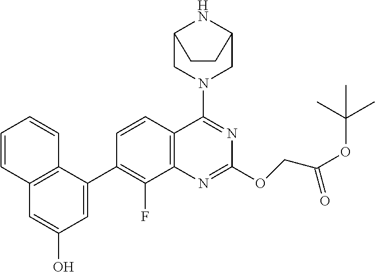 [SMILES: CC(C)(C)OC(=O)COc1nc(N2CC3CCC(C2)N3)c2ccc(-c3cc(O)cc4ccccc34)c(F)c2n1] | tert-butyl 2-((4-((1R,5S)-3,8- diazabicyclo[3.2.1]octan-3-yl)-8- fluoro-7-(3-hydroxynaphthalen-1- yl)quinazolin-2-yl)oxy)acetate | 531.2 |
| P-0745 | 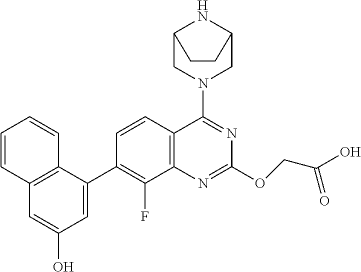 [SMILES: O=C(O)COc1nc(N2CC3CCC(C2)N3)c2ccc(-c3cc(O)cc4ccccc34)c(F)c2n1] | 2-((4-((1R,5S)-3,8- diazabicyclo[3.2.1]octan-3-yl)-8- fluoro-7-(3-hydroxynaphthalen-1- yl)quinazolin-2-yl)oxy)acetic acid | 475.1 |
| P-0746 | 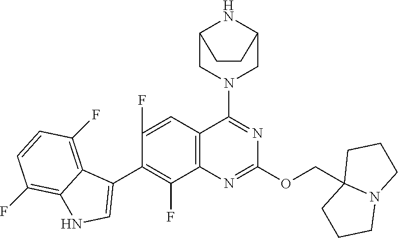 [SMILES: Fc1cc2c(N3CC4CCC(C3)N4)nc(OCC34CCCN3CCC4)nc2c(F)c1-c1c[nH]c2c(F)ccc(F)c12] | 4-((1R,5S)-3,8- diazabicyclo[3.2.1]octan-3-yl)-7-(4,7- difluoro-1H-indol-3-yl)-6,8-difluoro- 2-((tetrahydro-1H-pyrrolizin-7a(5H)- yl)methoxy)quinazoline | 567.6 |
| P-0747 | 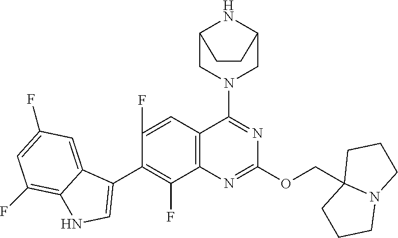 [SMILES: Fc1cc(F)c2[nH]cc(-c3c(F)cc4c(N5CC6CCC(C5)N6)nc(OCC56CCCN5CCC6)nc4c3F)c2c1] | 4-((1R,5S)-3,8- diazabicyclo[3.2.1]octan-3-yl)-7-(5,7- difluoro-1H-indol-3-yl)-6,8-difluoro- 2-((tetrahydro-1H-pyrrolizin-7a(5H)- yl)methoxy)quinazoline | 567.2 |
| P-0748 | 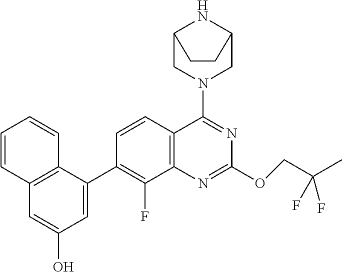 [SMILES: CC(F)(F)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-(2,2- difluoropropoxy)-8-fluoroquinazolin- 7-yl)naphthalen-2-ol | 495.2 |
| P-0749 | 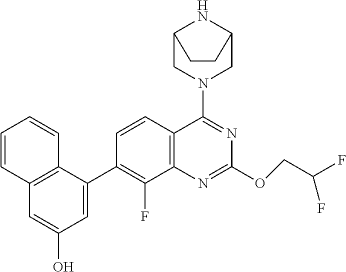 [SMILES: Oc1cc(-c2ccc3c(N4CC5CCC(C4)N5)nc(OCC(F)F)nc3c2F)c2ccccc2c1] | 4-(4-((1R,5S)-3,8- diazabicyclo[3.2.1]octan-3-yl)-2-(2,2- difluoroethoxy)-8-fluoroquinazolin-7- yl)naphthalen-2-ol | 481.2 |
| P-0750 | 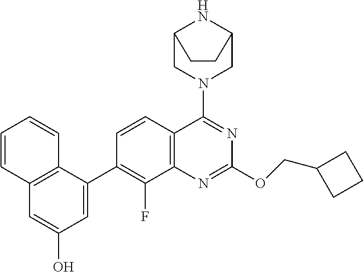 [SMILES: Oc1cc(-c2ccc3c(N4CC5CCC(C4)N5)nc(OCC4CCC4)nc3c2F)c2ccccc2c1] | 4-(4-((1R,5S)-3,8- diazabicyclo[3.2.1]octan-3-yl)-2- (cyclobutylmethoxy)-8- fluoroquinazolin-7-yl)naphthalen-2- ol | 485.3 |
| P-0751 | 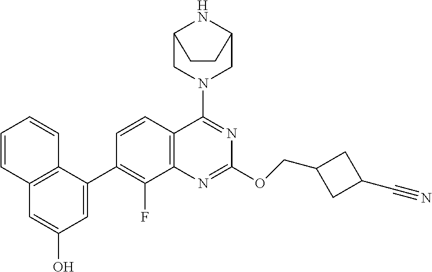 [SMILES: N#CC1CC(COc2nc(N3CC4CCC(C3)N4)c3ccc(-c4cc(O)cc5ccccc45)c(F)c3n2)C1] | 3-(((4-((1R,5S)-3,8- diazabicyclo[3.2.1]octan-3-yl)-8- fluoro-7-(3-hydroxynaphthalen-1- yl)quinazolin-2- yl)oxy)methyl)cyclobutane-1- carbonitrile | 510.3 |
| P-0752 | 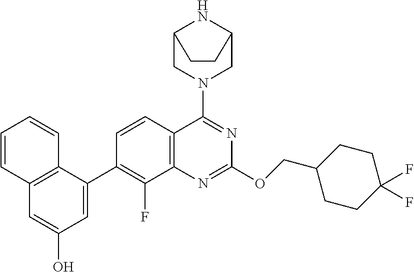 [SMILES: Oc1cc(-c2ccc3c(N4CC5CCC(C4)N5)nc(OCC4CCC(F)(F)CC4)nc3c2F)c2ccccc2c1] | 4-(4-((1R,5S)-3,8- diazabicyclo[3.2.1]octan-3-yl)-2- ((4,4-difluorocyclohexyl)methoxy)-8- fluoroquinazolin-7-yl)naphthalen-2- ol | 549.3 |
| P-0753 | 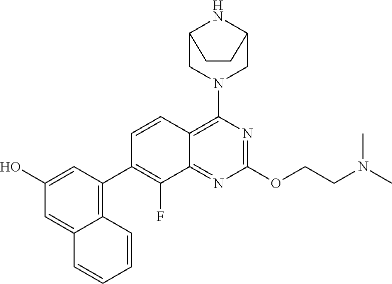 [SMILES: CN(C)CCOc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-(2- (dimethylamino)ethoxy)-8- fluoroquinazolin-7-yl)naphthalen-2- ol | 488.2 |
| P-0754 | 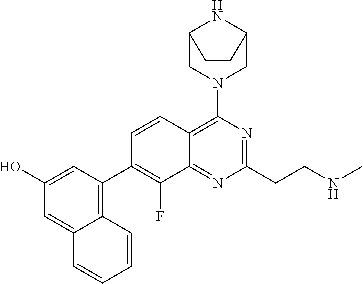 [SMILES: CNCCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2- (methylamino)ethyl)quinazolin-7- yl)naphthalen-2-ol | 458.1 |
| P-0755 | 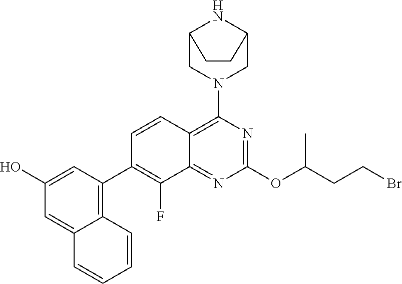 [SMILES: CC(CCBr)Oc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-2-((4- bromobutan-2-yl)oxy)-8- fluoroquinazolin-7-yl)naphthalen-2- ol | 549.1 |
| P-0756 | 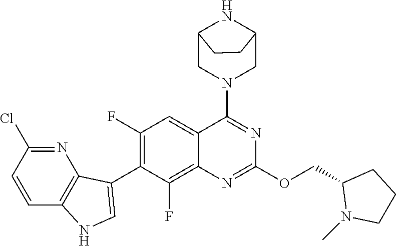 [SMILES: CN1CCC[C@H]1COc1nc(N2CC3CCC(C2)N3)c2cc(F)c(-c3c[nH]c4ccc(Cl)nc34)c(F)c2n1] | 4-((1R,5S)-3,8- diazabicyclo[3.2.1]octan-3-yl)-7-(5- chloro-1H-pyrrolo[3,2-b]pyridin-3- yl)-6,8-difluoro-2-(((S)-1- methylpyrrolidin-2- yl)methoxy)quinazoline | 540.4 |
| P-0757 | 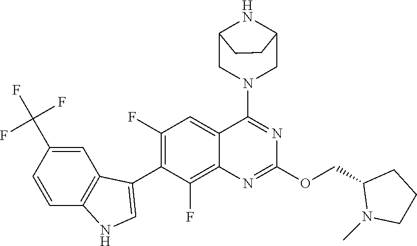 [SMILES: CN1CCC[C@H]1COc1nc(N2CC3CCC(C2)N3)c2cc(F)c(-c3c[nH]c4ccc(C(F)(F)F)cc34)c(F)c2n1] | 4-((1R,5S)-3,8- diazabicyclo[3.2.1]octan-3-yl)-6,8- difluoro-2-(((S)-1-methylpyrrolidin-2- yl)methoxy)-7-(5-(trifluoromethyl)- 1H-indol-3-yl)quinazoline | 573.1 |
| P-0758 | 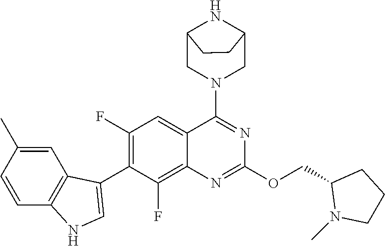 [SMILES: Cc1ccc2[nH]cc(-c3c(F)cc4c(N5CC6CCC(C5)N6)nc(OC[C@@H]5CCCN5C)nc4c3F)c2c1] | 4-((1R,5S)-3,8- diazabicyclo[3.2.1]octan-3-yl)-6,8- difluoro-7-(5-methyl-1H-indol-3-yl)- 2-(((S)-1-methylpyrrolidin-2- yl)methoxy)quinazoline | 519.7 |
| P-0759 | 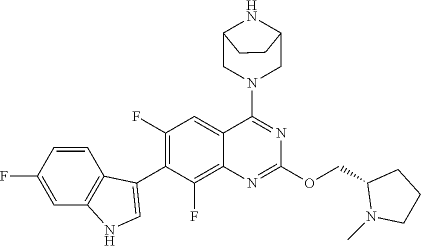 [SMILES: CN1CCC[C@H]1COc1nc(N2CC3CCC(C2)N3)c2cc(F)c(-c3c[nH]c4cc(F)ccc34)c(F)c2n1] | 4-((1R,5S)-3,8- diazabicyclo[3.2.1]octan-3-yl)-6,8- difluoro-7-(6-fluoro-1H-indol-3-yl)-2- (((S)-1-methylpyrrolidin-2- yl)methoxy)quinazoline | 523.3 |
| P-0760 | 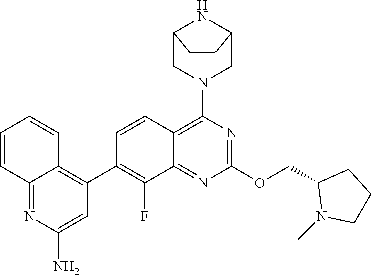 [SMILES: CN1CCC[C@H]1COc1nc(N2CC3CCC(C2)N3)c2ccc(-c3cc(N)nc4ccccc34)c(F)c2n1] | 4-(4-((1R,5S)-3,8- diazabicyclo[3.2.1]octan-3-yl)-8- fluoro-2-(((S)-1-methylpyrrolidin-2- yl)methoxy)quinazolin-7-yl)quinolin- 2-amine | 514.3 |
| P-0761 | 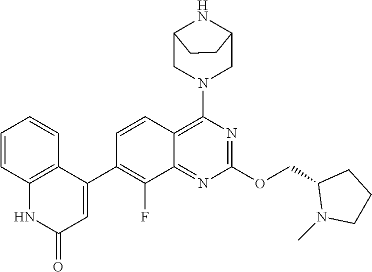 [SMILES: CN1CCC[C@H]1COc1nc(N2CC3CCC(C2)N3)c2ccc(-c3cc(=O)[nH]c4ccccc34)c(F)c2n1] | 4-(4-((1R,5S)-3,8- diazabicyclo[3.2.1]octan-3-yl)-8- fluoro-2-(((S)-1-methylpyrrolidin-2- yl)methoxy)quinazolin-7-yl)quinolin- 2(1H)-one | 515.2 |
| P-0762 | 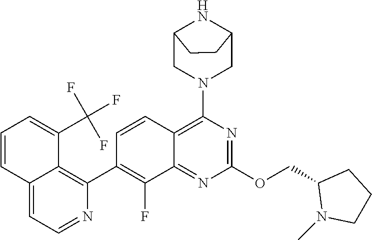 [SMILES: CN1CCC[C@H]1COc1nc(N2CC3CCC(C2)N3)c2ccc(-c3nccc4cccc(C(F)(F)F)c34)c(F)c2n1] | 4-((1R,5S)-3,8- diazabicyclo[3.2.1]octan-3-yl)-8- fluoro-2-(((S)-1-methylpyrrolidin-2- yl)methoxy)-7-(8- (trifluoromethyl)isoquinolin-1- yl)quinazoline | 567.4 |
| P-0763 | 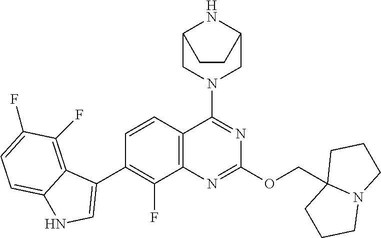 [SMILES: Fc1ccc2[nH]cc(-c3ccc4c(N5CC6CCC(C5)N6)nc(OCC56CCCN5CCC6)nc4c3F)c2c1F] | 4-((1R,5S)-3,8- diazabicyclo[3.2.1]octan-3-yl)-7-(4,5- difluoro-1H-indol-3-yl)-8-fluoro-2- ((tetrahydro-1H-pyrrolizin-7a(5H)- yl)methoxy)quinazoline | 549.3 |
| P-0764 | 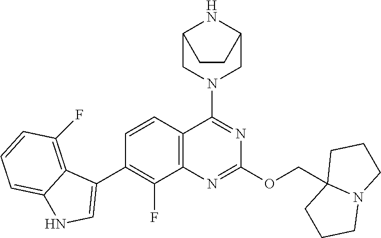 [SMILES: Fc1c(-c2c[nH]c3cccc(F)c23)ccc2c(N3CC4CCC(C3)N4)nc(OCC34CCCN3CCC4)nc12] | 4-((1R,5S)-3,8- diazabicyclo[3.2.1]octan-3-yl)-8- fluoro-7-(4-fluoro-1H-indol-3-yl)-2- ((tetrahydro-1H-pyrrolizin-7a(5H)- yl)methoxy)quinazoline | 531.3 |
| P-0765 | 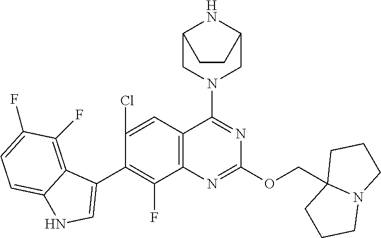 [SMILES: Fc1ccc2[nH]cc(-c3c(Cl)cc4c(N5CC6CCC(C5)N6)nc(OCC56CCCN5CCC6)nc4c3F)c2c1F] | 4-((1R,5S)-3,8- diazabicyclo[3.2.1]octan-3-yl)-6- chloro-7-(4,5-difluoro-1H-indol-3-yl)- 8-fluoro-2-((tetrahydro-1H-pyrrolizin- 7a(5H)-yl)methoxy)quinazoline | 583.2 |
| P-0766 | 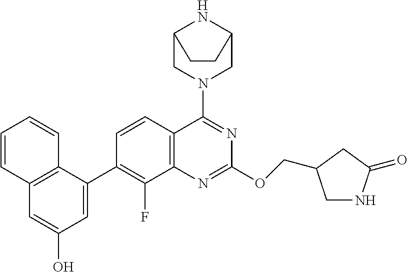 [SMILES: O=C1CC(COc2nc(N3CC4CCC(C3)N4)c3ccc(-c4cc(O)cc5ccccc45)c(F)c3n2)CN1] | 4-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-2-one | 514.3 |
| P-0767 | 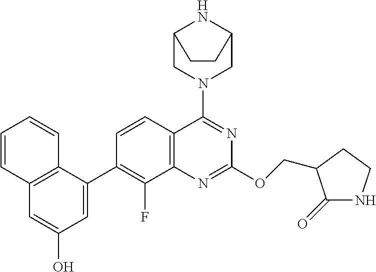 [SMILES: O=C1NCCC1COc1nc(N2CC3CCC(C2)N3)c2ccc(-c3cc(O)cc4ccccc34)c(F)c2n1] | 3-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-2-one | 514.2 |
| P-0768 | 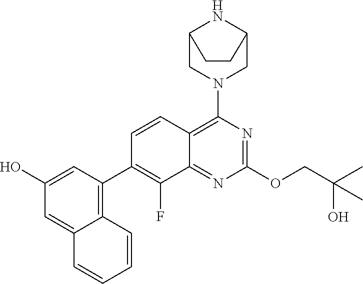 [SMILES: CC(C)(O)COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2-hydroxy-2- methylpropoxy)quinazolin-7- yl)naphthalen-2-ol | 489.6 |
| P-0769 | 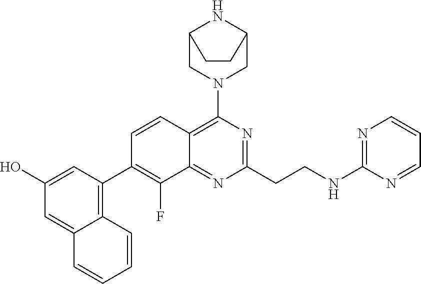 [SMILES: Oc1cc(-c2ccc3c(N4CC5CCC(C4)N5)nc(CCNc4ncccn4)nc3c2F)c2ccccc2c1] | 4-(4-((1R,5S)-3,8- diazabicyclo[3.2.1]octan-3-yl)-8- fluoro-2-(2-(pyrimidin-2- ylamino)ethyl)quinazolin-7- yl)naphthalen-2-ol | 522.2 |
| P-0770 | 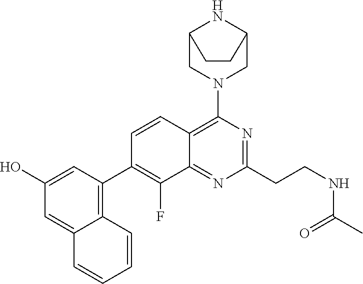 [SMILES: CC(=O)NCCc1nc(N2CC3CCC(C2)N3)c2ccc(-c3cc(O)cc4ccccc34)c(F)c2n1] | N-(2-(4-((1R,5S)-3,8- diazabicyclo[3.2.1]octan-3-yl)-8- fluoro-7-(3-hydroxynaphthalen-1- yl)quinazolin-2-yl)ethyl)acetamide | 486.2 |
| P-0771 | 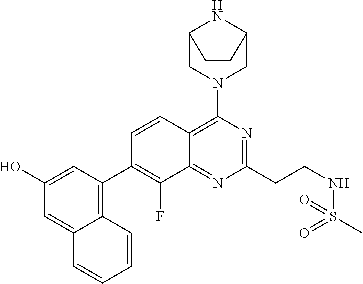 [SMILES: CS(=O)(=O)NCCc1nc(N2CC3CCC(C2)N3)c2ccc(-c3cc(O)cc4ccccc34)c(F)c2n1] | N-(2-(4-((1R,5S)-3,8- diazabicyclo[3.2.1]octan-3-yl)-8- fluoro-7-(3-hydroxynaphthalen-1- yl)quinazolin-2- yl)ethyl)methanesulfonamide | 522.2 |
| P-0772 | 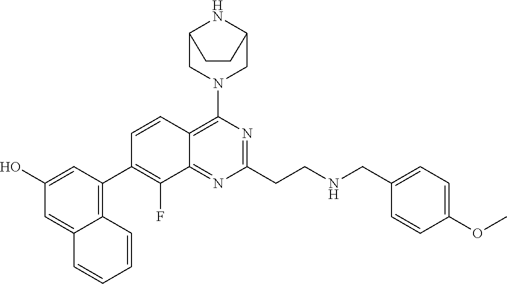 [SMILES: COc1ccc(CNCCc2nc(N3CC4CCC(C3)N4)c3ccc(-c4cc(O)cc5ccccc45)c(F)c3n2)cc1] | 4-(4-((1R,5S)-3,8- diazabicyclo[3.2.1]octan-3-yl)-8- fluoro-2-(2-((4- methoxybenzyl)amino)ethyl)quinazolin- 7-yl)naphthalen-2-ol | 564.2 |
| P-0773 | 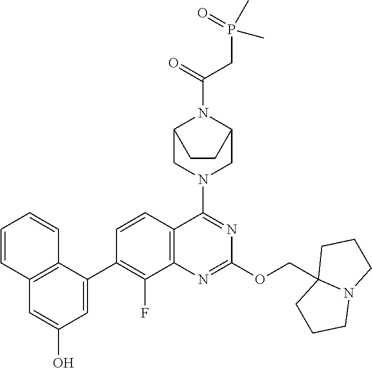 [SMILES: CP(C)(=O)CC(=O)N1C2CCC1CN(c1nc(OCC34CCCN3CCC4)nc3c(F)c(-c4cc(O)cc5ccccc45)ccc13)C2] | 2-(dimethylphosphoryl)-1-((1R,5S)-3- (8-fluoro-7-(3-hydroxynaphthalen-1- yl)-2-((tetrahydro-1H-pyrrolizin- 7a(5H)-yl)methoxy)quinazolin-4-yl)- 3,8-diazabicyclo[3.2.1]octan-8- yl)ethan-1-one | 658.2 |
| P-0774 | 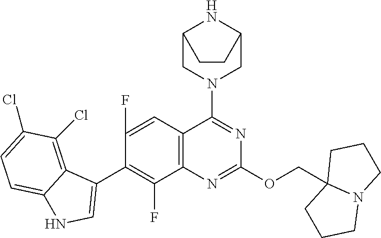 [SMILES: Fc1cc2c(N3CC4CCC(C3)N4)nc(OCC34CCCN3CCC4)nc2c(F)c1-c1c[nH]c2ccc(Cl)c(Cl)c12] | 4-((1R,5S)-3,8- diazabicyclo[3.2.1]octan-3-yl)-7-(4,5- dichloro-1H-indol-3-yl)-6,8-difluoro- 2-((tetrahydro-1H-pyrrolizin-7a(5H)- yl)methoxy)quinazoline | 597.1 |
| P-0775 | 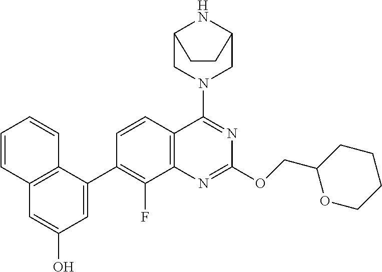 [SMILES: Oc1cc(-c2ccc3c(N4CC5CCC(C4)N5)nc(OCC4CCCCO4)nc3c2F)c2ccccc2c1] | 4-(4-((1R,5S)-3,8- diazabicyclo[3.2.1]octan-3-yl)-8- fluoro-2-((tetrahydro-2H-pyran-2- yl)methoxy)quinazolin-7- yl)naphthalen-2-ol | 515.3 |
| P-0776 | 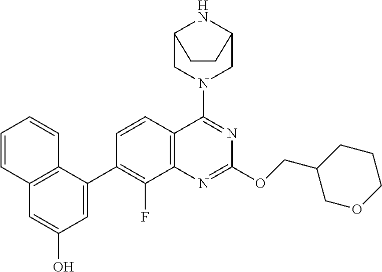 [SMILES: Oc1cc(-c2ccc3c(N4CC5CCC(C4)N5)nc(OCC4CCCOC4)nc3c2F)c2ccccc2c1] | 4-(4-((1R,5S)-3,8- diazabicyclo[3.2.1]octan-3-yl)-8- fluoro-2-((tetrahydro-2H-pyran-3- yl)methoxy)quinazolin-7- yl)naphthalen-2-ol | 515.3 |
| P-0777 | 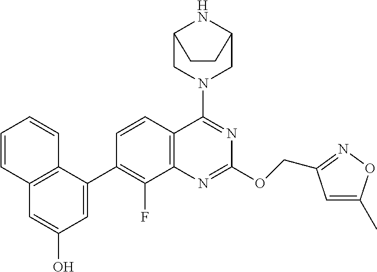 [SMILES: Cc1cc(COc2nc(N3CC4CCC(C3)N4)c3ccc(-c4cc(O)cc5ccccc45)c(F)c3n2)no1] | 4-(4-((1R,5S)-3,8- diazabicyclo[3.2.1]octan-3-yl)-8- fluoro-2-((5-methylisoxazol-3- yl)methoxy)quinazolin-7- yl)naphthalen-2-ol | 512.3 |
| P-0778 | 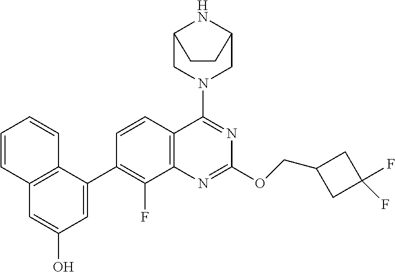 [SMILES: Oc1cc(-c2ccc3c(N4CC5CCC(C4)N5)nc(OCC4CC(F)(F)C4)nc3c2F)c2ccccc2c1] | 4-(4-((1R,5S)-3,8- diazabicyclo[3.2.1]octan-3-yl)-2- ((3,3-difluorocyclobutyl)methoxy)-8- fluoroquinazolin-7-yl)naphthalen-2- ol | 521.2 |
| P-0779 | 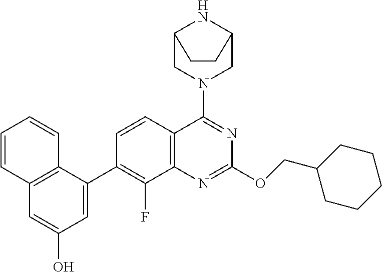 [SMILES: Oc1cc(-c2ccc3c(N4CC5CCC(C4)N5)nc(OCC4CCCCC4)nc3c2F)c2ccccc2c1] | 4-(4-((1R,5S)-3,8- diazabicyclo[3.2.1]octan-3-yl)-2- (cyclohexylmethoxy)-8- fluoroquinazolin-7-yl)naphthalen-2- ol | 513.3 |
| P-0780 | 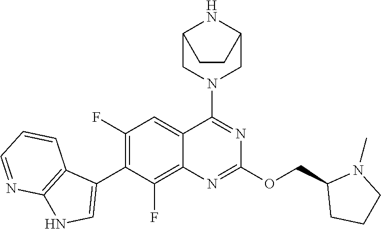 [SMILES: CN1CCC[C@H]1COc1nc(N2CC3CCC(C2)N3)c2cc(F)c(-c3c[nH]c4ncccc34)c(F)c2n1] | 4-((1R,5S)-3,8- diazabicyclo[3.2.1]octan-3-yl)-6,8- difluoro-2-(((S)-1-methylpyrrolidin-2- yl)methoxy)-7-(1H-pyrrolo[2,3- b]pyridin-3-yl)quinazoline | 506.2 |
| P-0781 | 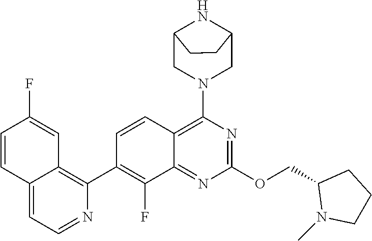 [SMILES: CN1CCC[C@H]1COc1nc(N2CC3CCC(C2)N3)c2ccc(-c3nccc4ccc(F)cc34)c(F)c2n1] | 4-((1R,5S)-3,8- diazabicyclo[3.2.1]octan-3-yl)-8- fluoro-7-(7-fluoroisoquinolin-1-yl)-2- (((S)-1-methylpyrrolidin-2- yl)methoxy)quinazoline | 517.6 |
| P-0782 | 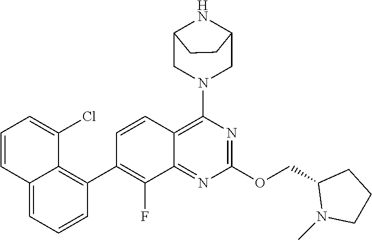 [SMILES: CN1CCC[C@H]1COc1nc(N2CC3CCC(C2)N3)c2ccc(-c3cccc4cccc(Cl)c34)c(F)c2n1] | 4-((1R,5S)-3,8- diazabicyclo[3.2.1]octan-3-yl)-7-(8- chloronaphthalen-1-yl)-8-fluoro-2- (((S)-1-methylpyrrolidin-2- yl)methoxy)quinazoline | 532.0 |
| P-0783 | 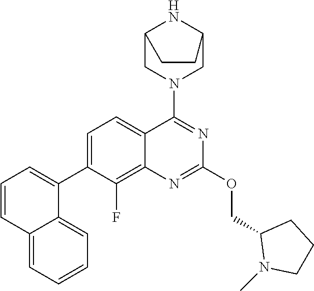 [SMILES: CN1CCC[C@H]1COc1nc(N2CC3CCC(C2)N3)c2ccc(-c3cccc4ccccc34)c(F)c2n1] | 4-((1R,5S)-3,8- diazabicyclo[3.2.1]octan-3-yl)-8- fluoro-2-(((S)-1-methylpyrrolidin-2- yl)methoxy)-7-(naphthalen-1- yl)quinazoline | 498.7 |
| P-0784 | 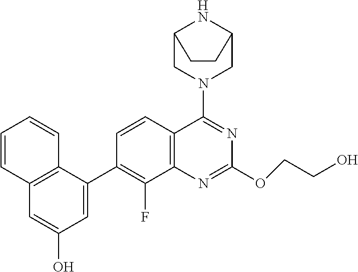 [SMILES: OCCOc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(2- hydroxyethoxy)quinazolin-7- yl)naphthalen-2-ol | 461.3 |
| P-0785 | 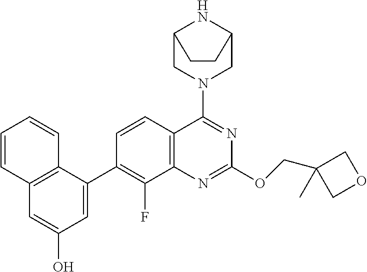 [SMILES: CC1(COc2nc(N3CC4CCC(C3)N4)c3ccc(-c4cc(O)cc5ccccc45)c(F)c3n2)COC1] | 4-(4-((1R,5S)-3,8- diazabicyclo[3.2.1]octan-3-yl)-8- fluoro-2-((3-methyloxetan-3- yl)methoxy)quinazolin-7- yl)naphthalen-2-ol | 501.3 |
| P-0786 | 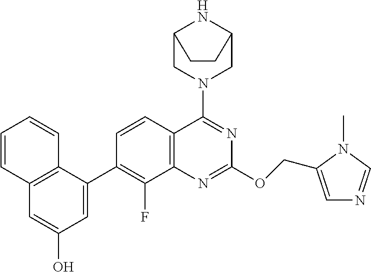 [SMILES: Cn1cncc1COc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-((1-methyl-1H-imidazol-5- yl)methoxy)quinazolin-7- yl)naphthalen-2-ol | 511.3 |
| P-0787 | 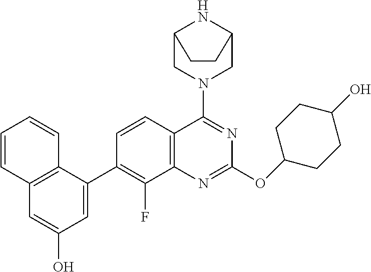 [SMILES: Oc1cc(-c2ccc3c(N4CC5CCC(C4)N5)nc(OC4CCC(O)CC4)nc3c2F)c2ccccc2c1] | 4-(4-((1R,5S)-3,8- diazabicyclo[3.2.1]octan-3-yl)-8- fluoro-2-((4- hydroxycyclohexyl)oxy)quinazolin-7- yl)naphthalen-2-ol | 515.3 |
| P-0788 | 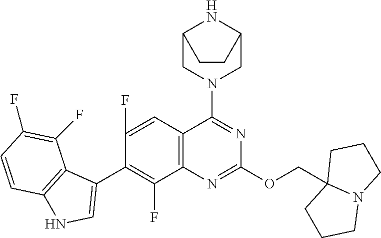 [SMILES: Fc1cc2c(N3CC4CCC(C3)N4)nc(OCC34CCCN3CCC4)nc2c(F)c1-c1c[nH]c2ccc(F)c(F)c12] | 4-((1R,5S)-3,8- diazabicyclo[3.2.1]octan-3-yl)-7-(4,5- difluoro-1H-indol-3-yl)-6,8-difluoro- 2-((tetrahydro-1H-pyrrolizin-7a(5H)- yl)methoxy)quinazoline | 567.3 |
| P-0789 | 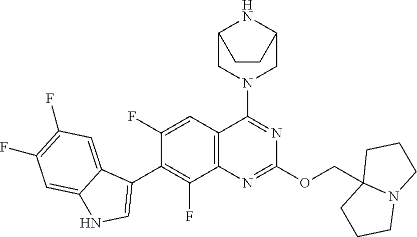 [SMILES: Fc1cc2[nH]cc(-c3c(F)cc4c(N5CC6CCC(C5)N6)nc(OCC56CCCN5CCC6)nc4c3F)c2cc1F] | 4-((1R,5S)-3,8- diazabicyclo[3.2.1]octan-3-yl)-7-(5,6- difluoro-1H-indol-3-yl)-6,8-difluoro- 2-((tetrahydro-1H-pyrrolizin-7a(5H)- yl)methoxy)quinazoline | 567.6 |
| P-0790 | 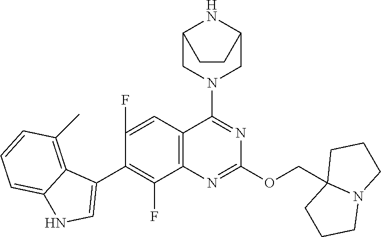 [SMILES: Cc1cccc2[nH]cc(-c3c(F)cc4c(N5CC6CCC(C5)N6)nc(OCC56CCCN5CCC6)nc4c3F)c12] | 4-((1R,5S)-3,8- diazabicyclo[3.2.1]octan-3-yl)-6,8- difluoro-7-(4-methyl-1H-indol-3-yl)- 2-((tetrahydro-1H-pyrrolizin-7a(5H)- yl)methoxy)quinazoline | 545.7 |
| P-0791 | 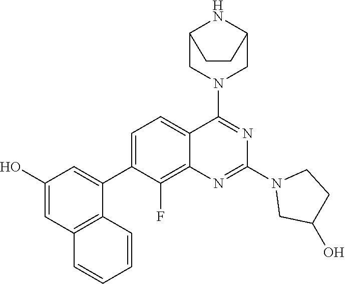 [SMILES: Oc1cc(-c2ccc3c(N4CC5CCC(C4)N5)nc(N4CCC(O)C4)nc3c2F)c2ccccc2c1] | 1-(4-((1R,5S)-3,8- diazabicyclo[3.2.1]octan-3-yl)-8- fluoro-7-(3-hydroxynaphthalen-1- yl)quinazolin-2-yl)pyrrolidin-3-ol | 486.1 |
| P-0792 | 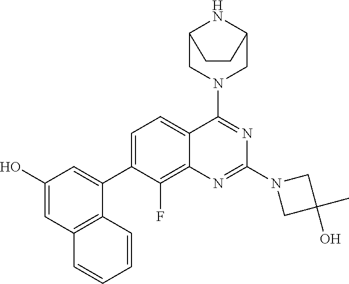 [SMILES: CC1(O)CN(c2nc(N3CC4CCC(C3)N4)c3ccc(-c4cc(O)cc5ccccc45)c(F)c3n2)C1] | 1-(4-((1R,5S)-3,8- diazabicyclo[3.2.1]octan-3-yl)-8- fluoro-7-(3-hydroxynaphthalen-1- yl)quinazolin-2-yl)-3-methylazetidin- 3-ol | 484.2 |
| P-0793 | 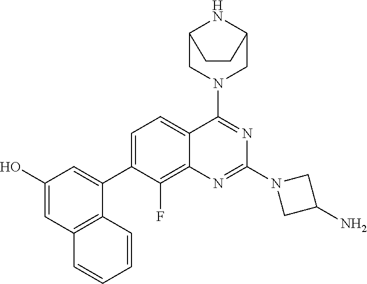 [SMILES: NC1CN(c2nc(N3CC4CCC(C3)N4)c3ccc(-c4cc(O)cc5ccccc45)c(F)c3n2)C1] | 4-(2-(3-aminoazetidin-1-yl)-4- ((1R,5S)-3,8- diazabicyclo[3.2.1]octan-3-yl)-8- fluoroquinazolin-7-yl)naphthalen-2- ol | 471.2 |
| P-0794 | 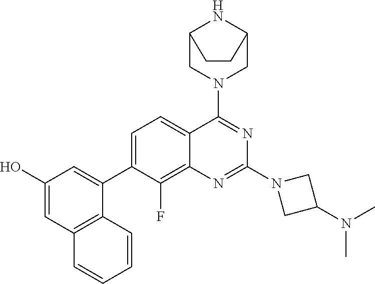 [SMILES: CN(C)C1CN(c2nc(N3CC4CCC(C3)N4)c3ccc(-c4cc(O)cc5ccccc45)c(F)c3n2)C1] | 4-(4-((1R,5S)-3,8- diazabicyclo[3.2.1]octan-3-yl)-2-(3- (dimethylamino)azetidin-1-yl)-8- fluoroquinazolin-7-yl)naphthalen-2- ol | 499.6 |
| P-0795 | 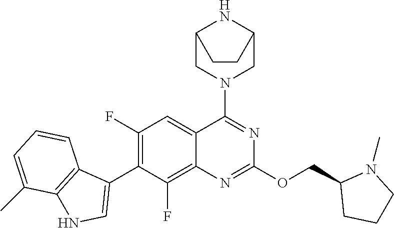 [SMILES: Cc1cccc2c(-c3c(F)cc4c(N5CC6CCC(C5)N6)nc(OC[C@@H]5CCCN5C)nc4c3F)c[nH]c12] | 4-((1R,5S)-3,8- diazabicyclo[3.2.1]octan-3-yl)-6,8- difluoro-7-(7-methyl-1H-indol-3-yl)- 2-(((S)-1-methylpyrrolidin-2- yl)methoxy)quinazoline | 519.4 |
| P-0796 | 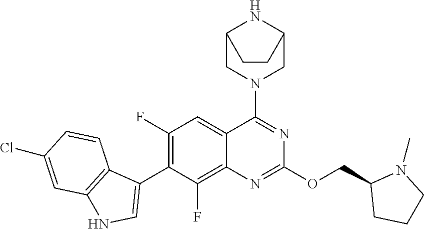 [SMILES: CN1CCC[C@H]1COc1nc(N2CC3CCC(C2)N3)c2cc(F)c(-c3c[nH]c4cc(Cl)ccc34)c(F)c2n1] | 4-((1R,5S)-3,8- diazabicyclo[3.2.1]octan-3-yl)-7-(6- chloro-1H-indol-3-yl)-6,8-difluoro-2- (((S)-1-methylpyrrolidin-2- yl)methoxy)quinazoline | 539.5 |
| P-0797 | 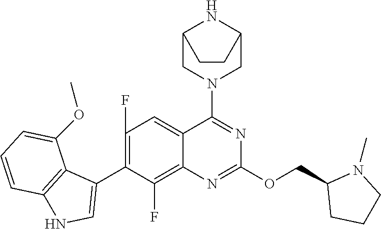 [SMILES: COc1cccc2[nH]cc(-c3c(F)cc4c(N5CC6CCC(C5)N6)nc(OC[C@@H]5CCCN5C)nc4c3F)c12] | 4-((1R,5S)-3,8- diazabicyclo[3.2.1]octan-3-yl)-6,8- difluoro-7-(4-methoxy-1H-indol-3- yl)-2-(((S)-1-methylpyrrolidin-2- yl)methoxy)quinazoline | 535.3 |
| P-0798 | 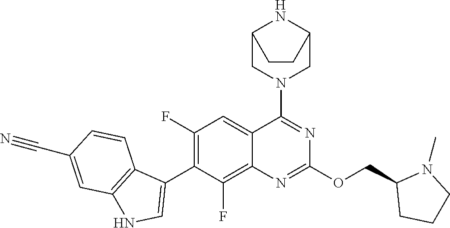 [SMILES: CN1CCC[C@H]1COc1nc(N2CC3CCC(C2)N3)c2cc(F)c(-c3c[nH]c4cc(C#N)ccc34)c(F)c2n1] | 3-(4-((1R,5S)-3,8- diazabicyclo[3.2.1]octan-3-yl)-6,8- difluoro-2-(((S)-1-methylpyrrolidin-2- yl)methoxy)quinazolin-7-yl)-1H- indole-6-carbonitrile | 530.2 |
| P-0799 | 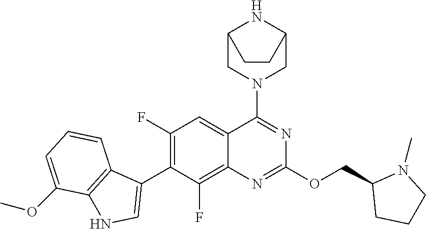 [SMILES: COc1cccc2c(-c3c(F)cc4c(N5CC6CCC(C5)N6)nc(OC[C@@H]5CCCN5C)nc4c3F)c[nH]c12] | 4-((1R,5S)-3,8- diazabicyclo[3.2.1]octan-3-yl)-6,8- difluoro-7-(7-methoxy-1H-indol-3- yl)-2-(((S)-1-methylpyrrolidin-2- yl)methoxy)quinazoline | 535.3 |
| P-0800 | 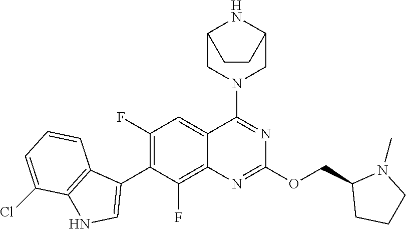 [SMILES: CN1CCC[C@H]1COc1nc(N2CC3CCC(C2)N3)c2cc(F)c(-c3c[nH]c4c(Cl)cccc34)c(F)c2n1] | 4-((1R,5S)-3,8- diazabicyclo[3.2.1]octan-3-yl)-7-(7- chloro-1H-indol-3-yl)-6,8-difluoro-2- (((S)-1-methylpyrrolidin-2- yl)methoxy)quinazoline | 539.2 |
| P-0801 | 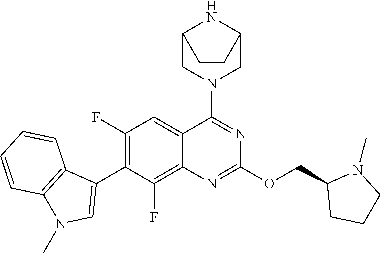 [SMILES: CN1CCC[C@H]1COc1nc(N2CC3CCC(C2)N3)c2cc(F)c(-c3cn(C)c4ccccc34)c(F)c2n1] | 4-((1R,5S)-3,8- diazabicyclo[3.2.1]octan-3-yl)-6,8- difluoro-7-(1-methyl-1H-indol-3-yl)- 2-(((S)-1-methylpyrrolidin-2- yl)methoxy)quinazoline | 519.7 |
| P-0802 | 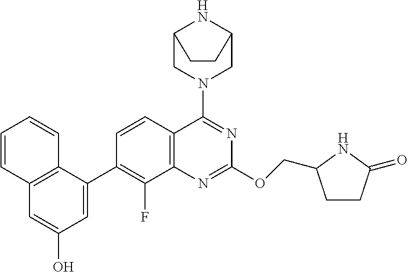 [SMILES: O=C1CCC(COc2nc(N3CC4CCC(C3)N4)c3ccc(-c4cc(O)cc5ccccc45)c(F)c3n2)N1] | 5-(((4-((1R,5S)-3,8- diazabicyclo[3.2.1]octan-3-yl)-8- fluoro-7-(3-hydroxynaphthalen-1- yl)quinazolin-2- yl)oxy)methyl)pyrrolidin-2-one | 514.3 |
| P-0803 | 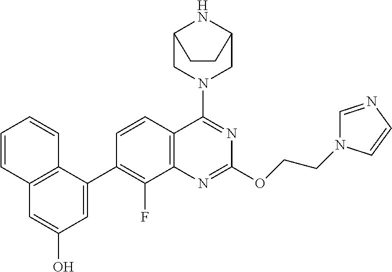 [SMILES: Oc1cc(-c2ccc3c(N4CC5CCC(C4)N5)nc(OCCn4ccnc4)nc3c2F)c2ccccc2c1] | 4-(2-(2-(1H-imidazol-1-yl)ethoxy)-4- ((1R,5S)-3,8- diazabicyclo[3.2.1]octan-3-yl)-8- fluoroquinazolin-7-yl)naphthalen-2- ol | 511.3 |
| P-0804 | 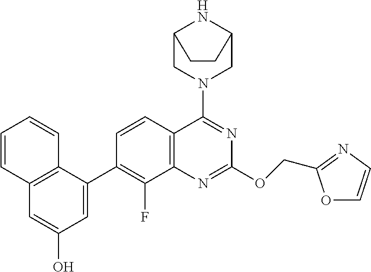 [SMILES: Oc1cc(-c2ccc3c(N4CC5CCC(C4)N5)nc(OCc4ncco4)nc3c2F)c2ccccc2c1] | 4-(4-((1R,5S)-3,8- diazabicyclo[3.2.1]octan-3-yl)-8- fluoro-2-(oxazol-2- ylmethoxy)quinazolin-7- yl)naphthalen-2-ol | 498.2 |
| P-0805 | 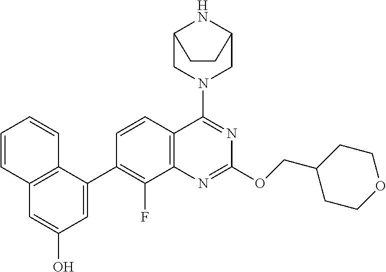 [SMILES: Oc1cc(-c2ccc3c(N4CC5CCC(C4)N5)nc(OCC4CCOCC4)nc3c2F)c2ccccc2c1] | 4-(4-((1R,5S)-3,8- diazabicyclo[3.2.1]octan-3-yl)-8- fluoro-2-((tetrahydro-2H-pyran-4- yl)methoxy)quinazolin-7- yl)naphthalen-2-ol | 515.3 |
| P-0806 | 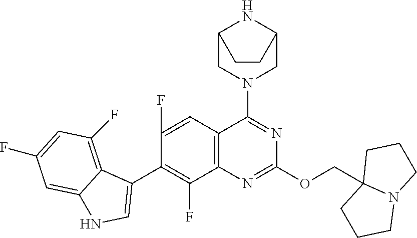 [SMILES: Fc1cc(F)c2c(-c3c(F)cc4c(N5CC6CCC(C5)N6)nc(OCC56CCCN5CCC6)nc4c3F)c[nH]c2c1] | 4-((1R,5S)-3,8- diazabicyclo[3.2.1]octan-3-yl)-7-(4,6- difluoro-1H-indol-3-yl)-6,8-difluoro- 2-((tetrahydro-1H-pyrrolizin-7a(5H)- yl)methoxy)quinazoline | 567.6 |
| P-0807 | 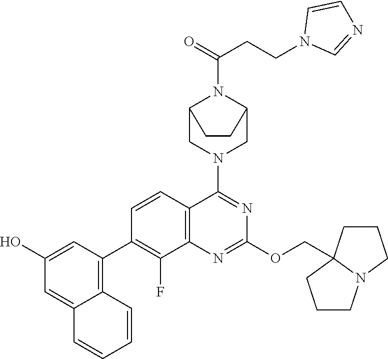 [SMILES: O=C(CCn1ccnc1)N1C2CCC1CN(c1nc(OCC34CCCN3CCC4)nc3c(F)c(-c4cc(O)cc5ccccc45)ccc13)C2] | 1-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3-(1H- imidazol-1-yl)propan-1-one | 662.4 |
| P-0808 | 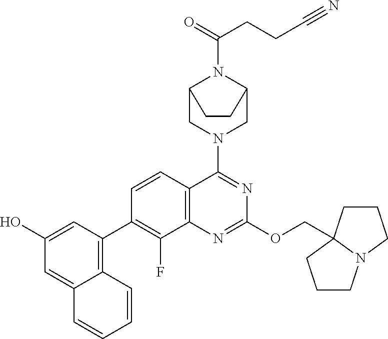 [SMILES: N#CCCC(=O)N1C2CCC1CN(c1nc(OCC34CCCN3CCC4)nc3c(F)c(-c4cc(O)cc5ccccc45)ccc13)C2] | 4-((1R,5S)-3-(8-fluoro-7-(3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-4- oxobutanenitrile | 621.4 |
| P-0809 | 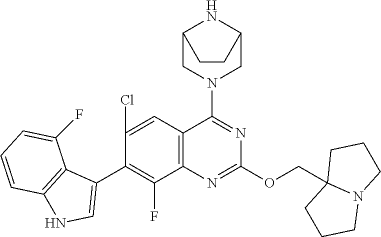 [SMILES: Fc1c(-c2c[nH]c3cccc(F)c23)c(Cl)cc2c(N3CC4CCC(C3)N4)nc(OCC34CCCN3CCC4)nc12] | 4-((1R,5S)-3,8- diazabicyclo[3.2.1]octan-3-yl)-6- chloro-8-fluoro-7-(4-fluoro-1H-indol- 3-yl)-2-((tetrahydro-1H-pyrrolizin- 7a(5H)-yl)methoxy)quinazoline | 565.3 |
| P-0810 | 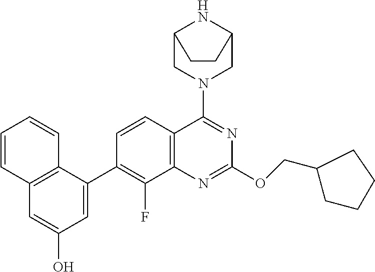 [SMILES: Oc1cc(-c2ccc3c(N4CC5CCC(C4)N5)nc(OCC4CCCC4)nc3c2F)c2ccccc2c1] | 4-(4-((1R,5S)-3,8- diazabicyclo[3.2.1]octan-3-yl)-2- (cyclopentylmethoxy)-8- fluoroquinazolin-7-yl)naphthalen-2- ol | 499.3 |
| P-0811 | 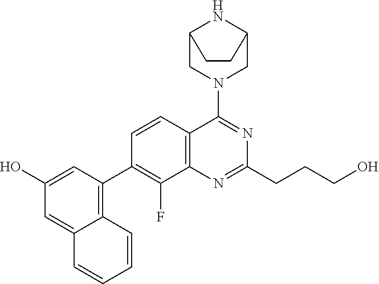 [SMILES: OCCCc1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-(3- hydroxypropyl)quinazolin-7- yl)naphthalen-2-ol | 459.3 |
| P-0812 | 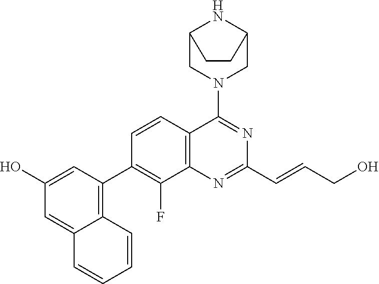 [SMILES: OC/C=C/c1nc(N2CC3CCC(C2)N3)c2ccc(-c3cc(O)cc4ccccc34)c(F)c2n1] | 4-(4-((1R,5S)-3,8- diazabicyclo[3.2.1]octan-3-yl)-8- fluoro-2-((E)-3-hydroxyprop-1-en-1- yl)quinazolin-7-yl)naphthalen-2-ol | 457.3 |
| P-0813 | 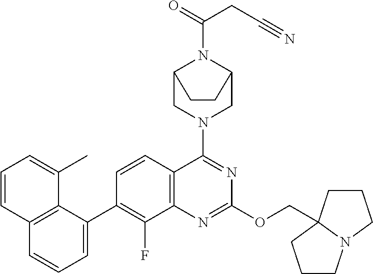 [SMILES: Cc1cccc2cccc(-c3ccc4c(N5CC6CCC(C5)N6C(=O)CC#N)nc(OCC56CCCN5CCC6)nc4c3F)c12] | 3-((1R,5S)-3-(8-fluoro-7-(8- methylnaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- oxopropanenitrile | 605.4 |
| P-0814 | 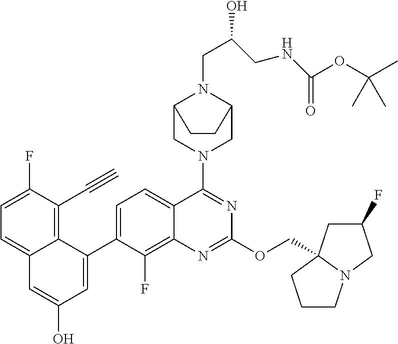 [SMILES: C#Cc1c(F)ccc2cc(O)cc(-c3ccc4c(N5CC6CCC(C5)N6C[C@H](O)CNC(=O)OC(C)(C)C)nc(OC[C@@]56CCCN5C[C@H](F)C6)nc4c3F)c12] | tert-butyl ((R)-3-((1R,5S)-3-(7-(8- ethynyl-7-fluoro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)carbamate | 772.0 |
| P-0815 | 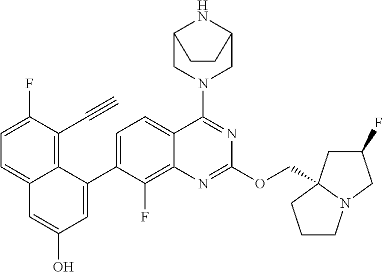 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-ethynyl-6-fluoronaphthalen-2-ol | 599.0 |
| P-0816 | 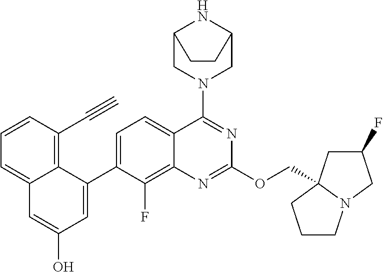 [SMILES: C#Cc1cccc2cc(O)cc(-c3ccc4c(N5CC6CCC(C5)N6)nc(OC[C@@]56CCCN5C[C@H](F)C6)nc4c3F)c12] | 4-(4-((1R,5S)-3,8- diazabicyclo[3.2.1]octan-3-yl)-8- fluoro-2-(((2R,7aS)-2- fluorotetrahydro-1H-pyrrolizin- 7a(5H)-yl)methoxy)quinazolin-7-yl)- 5-ethynylnaphthalen-2-ol | 581.0 |
| P-0817 | 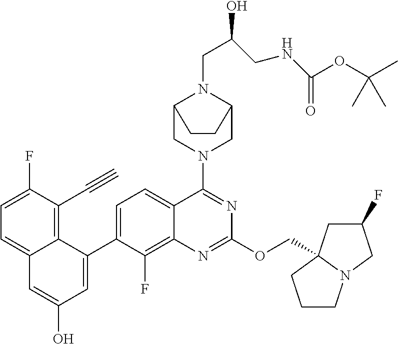 [SMILES: C#Cc1c(F)ccc2cc(O)cc(-c3ccc4c(N5CC6CCC(C5)N6C[C@@H](O)CNC(=O)OC(C)(C)C)nc(OC[C@@]56CCCN5C[C@H](F)C6)nc4c3F)c12] | tert-butyl ((S)-3-((1R,5S)-3-(7-(8- ethynyl-7-fluoro-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)carbamate | 772.0 |
| P-0818 | 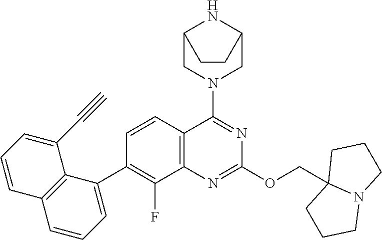 [SMILES: C#Cc1cccc2cccc(-c3ccc4c(N5CC6CCC(C5)N6)nc(OCC56CCCN5CCC6)nc4c3F)c12] | 4-((1R,5S)-3,8- diazabicyclo[3.2.1]octan-3-yl)-7-(8- ethynylnaphthalen-1-yl)-8-fluoro-2- ((tetrahydro-1H-pyrrolizin-7a(5H)- yl)methoxy)quinazoline | 548.4 |
| P-0819 | 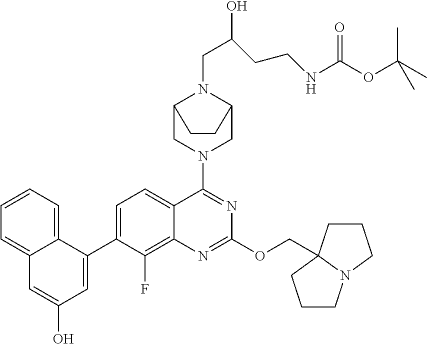 [SMILES: CC(C)(C)OC(=O)NCCC(O)CN1C2CCC1CN(c1nc(OCC34CCCN3CCC4)nc3c(F)c(-c4cc(O)cc5ccccc45)ccc13)C2] | tert-butyl (4-((1R,5S)-3-(8-fluoro-7- (3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-3- hydroxybutyl)carbamate | 727.0 |
| P-0820 | 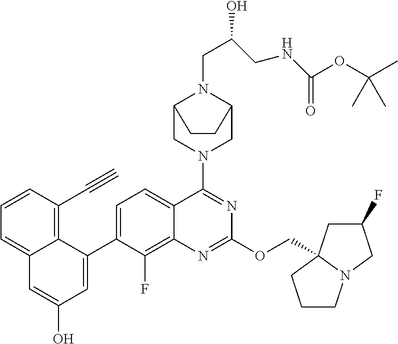 [SMILES: C#Cc1cccc2cc(O)cc(-c3ccc4c(N5CC6CCC(C5)N6C[C@H](O)CNC(=O)OC(C)(C)C)nc(OC[C@@]56CCCN5C[C@H](F)C6)nc4c3F)c12] | tert-butyl ((R)-3-((1R,5S)-3-(7-(8- ethynyl-3-hydroxynaphthalen-1-yl)-8- fluoro-2-(((2R,7aS)-2- fluorotetrahydro-1H-pyrrolizin- 7a(5H)-yl)methoxy)quinazolin-4-yl)- 3,8-diazabicyclo[3.2.1]octan-8-yl)-2- hydroxypropyl)carbamate | 755.0 |
| P-0821 | 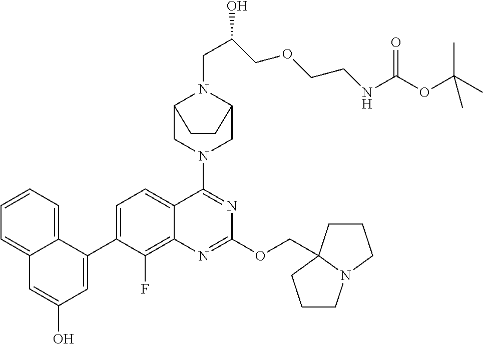 [SMILES: CC(C)(C)OC(=O)NCCOC[C@@H](O)CN1C2CCC1CN(c1nc(OCC34CCCN3CCC4)nc3c(F)c(-c4cc(O)cc5ccccc45)ccc13)C2] | tert-butyl (2-(3-((1R,5S)-3-(8-fluoro- 7-(3-hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)-2- hydroxypropoxy)ethyl)carbamate | 757.0 |
| P-0822 | 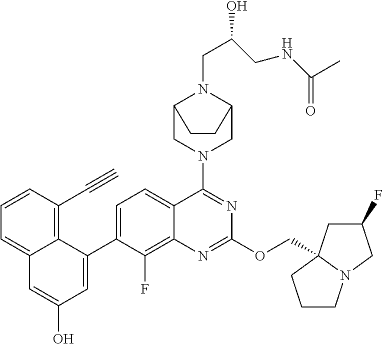 [SMILES: C#Cc1cccc2cc(O)cc(-c3ccc4c(N5CC6CCC(C5)N6C[C@H](O)CNC(C)=O)nc(OC[C@@]56CCCN5C[C@H](F)C6)nc4c3F)c12] | N-((R)-3-((1R,5S)-3-(7-(8-ethynyl-3- hydroxynaphthalen-1-yl)-8-fluoro-2- (((2R,7aS)-2-fluorotetrahydro-1H- pyrrolizin-7a(5H)- yl)methoxy)quinazolin-4-yl)-3,8- diazabicyclo[3.2.1]octan-8-yl)-2- hydroxypropyl)acetamide | 697.0 |
| P-0823 | 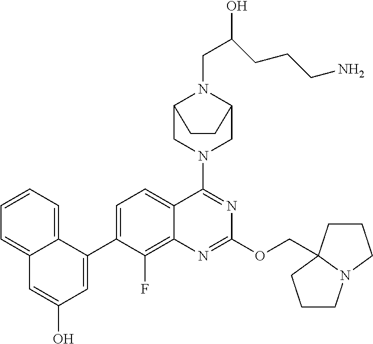 [SMILES: NCCCC(O)CN1C2CCC1CN(c1nc(OCC34CCCN3CCC4)nc3c(F)c(-c4cc(O)cc5ccccc45)ccc13)C2] | 4-(4-((1R,5S)-8-(5-amino-2- hydroxypentyl)-3,8- diazabicyclo[3.2.1]octan-3-yl)-8- fluoro-2-((tetrahydro-1H-pyrrolizin- 7a(5H)-yl)methoxy)quinazolin-7- yl)naphthalen-2-ol | 641.0 |
| P-0824 | 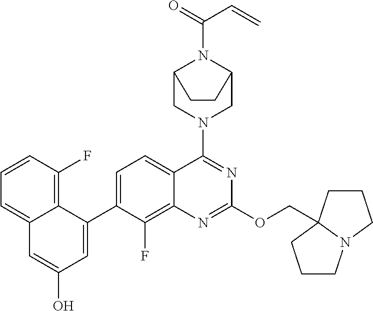 [SMILES: C=CC(=O)N1C2CCC1CN(c1nc(OCC34CCCN3CCC4)nc3c(F)c(-c4cc(O)cc5cccc(F)c45)ccc13)C2] | 1-((1R,5S)-3-(8-fluoro-7-(8-fluoro-3- hydroxynaphthalen-1-yl)-2- ((tetrahydro-1H-pyrrolizin-7a(5H)- yl)methoxy)quinazolin-4-yl)-3,8- diazabicyclo[3.2.1]octan-8-yl)prop-2- en-1-one | 612.0 |
| P-0825 | 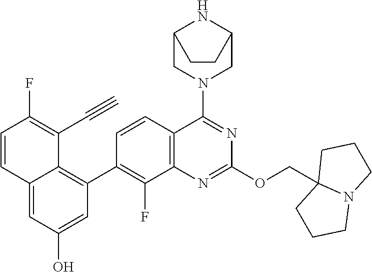 [SMILES: C#Cc1c(F)ccc2cc(O)cc(-c3ccc4c(N5CC6CCC(C5)N6)nc(OCC56CCCN5CCC6)nc4c3F)c12] | 4-(4-((1R,5S)-3,8- diazabicyclo[3.2.1]octan-3-yl)-8- fluoro-2-((tetrahydro-1H-pyrrolizin- 7a(5H)-yl)methoxy)quinazolin-7-yl)- 5-ethynyl-6-fluoronaphthalen-2-ol | 582.0 |

TABLE IB

| P# | Structure | Name |
|---|---|---|
| P-0826 | 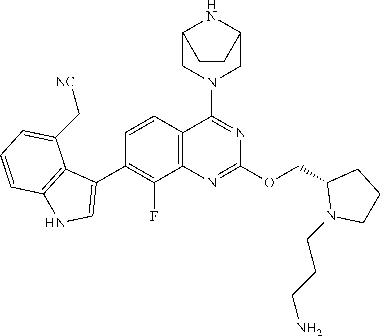 [SMILES: N#CCc1cccc2[nH]cc(-c3ccc4c(N5CC6CCC(C5)N6)nc(OC[C@@H]5CCCN5CCCN)nc4c3F)c12] | 2-(3-(2-(((S)-1-(3- aminopropyl)pyrrolidin-2- yl)methoxy)-4-((1R,5S)-3,8- diazabicyclo[3.2.1]octan-3-yl)-8- fluoroquinazolin-7-yl)-1H-indol-4- yl)acetonitrile |
| P-0827 | 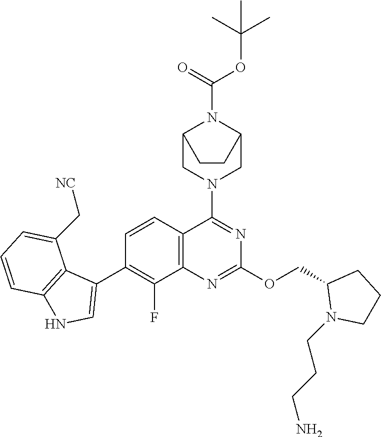 [SMILES: CC(C)(C)OC(=O)N1C2CCC1CN(c1nc(OC[C@@H]3CCCN3CCCN)nc3c(F)c(-c4c[nH]c5cccc(CC#N)c45)ccc13)C2] | tert-butyl (1R,5S)-3-(2-(((S)-1-(3- aminopropyl)pyrrolidin-2- yl)methoxy)-7-(4-(cyanomethyl)- 1H-indol-3-yl)-8-fluoroquinazolin- 4-yl)-3,8-diazabicyclo[3.2.1]octane- 8-carboxylate |
| P-0828 | 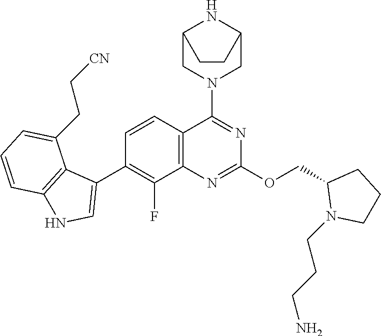 [SMILES: N#CCCc1cccc2[nH]cc(-c3ccc4c(N5CC6CCC(C5)N6)nc(OC[C@@H]5CCCN5CCCN)nc4c3F)c12] | 3-(3-(2-(((S)-1-(3- aminopropyl)pyrrolidin-2- yl)methoxy)-4-((1R,5S)-3,8- diazabicyclo[3.2.1]octan-3-yl)-8- fluoroquinazolin-7-yl)-1H-indol-4- yl)propanenitrile |
| P-0829 | 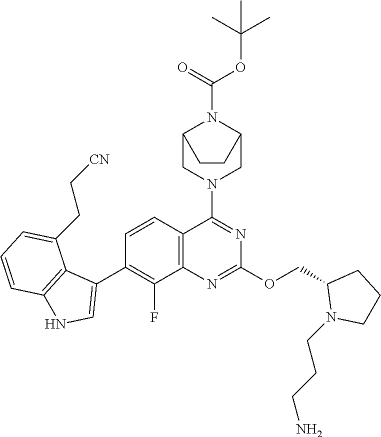 [SMILES: CC(C)(C)OC(=O)N1C2CCC1CN(c1nc(OC[C@@H]3CCCN3CCCN)nc3c(F)c(-c4c[nH]c5cccc(CCC#N)c45)ccc13)C2] | tert-butyl (1R,5S)-3-(2-(((S)-1-(3- aminopropyl)pyrrolidin-2- yl)methoxy)-7-(4-(2-cyanoethyl)- 1H-indol-3-yl)-8-fluoroquinazolin- 4-yl)-3,8-diazabicyclo[3.2.1]octane- 8-carboxylate |
| P-0830 | 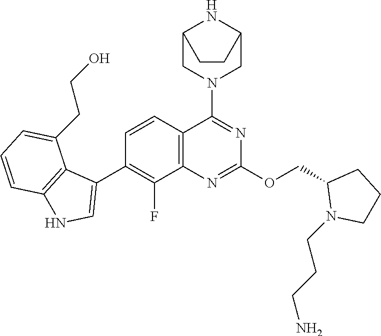 [SMILES: NCCCN1CCC[C@H]1COc1nc(N2CC3CCC(C2)N3)c2ccc(-c3c[nH]c4cccc(CCO)c34)c(F)c2n1] | 2-(3-(2-(((S)-1-(3- aminopropyl)pyrrolidin-2- yl)methoxy)-4-((1R,5S)-3,8- diazabicyclo[3.2.1]octan-3-yl)-8- fluoroquinazolin-7-yl)-1H-indol-4- yl)ethan-1-ol |
| P-0831 | 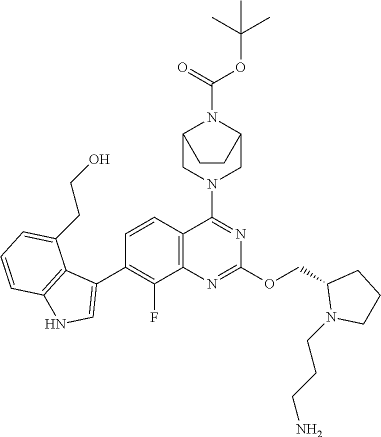 [SMILES: CC(C)(C)OC(=O)N1C2CCC1CN(c1nc(OC[C@@H]3CCCN3CCCN)nc3c(F)c(-c4c[nH]c5cccc(CCO)c45)ccc13)C2] | tert-butyl (1R,5S)-3-(2-(((S)-1-(3- aminopropyl)pyrrolidin-2- yl)methoxy)-8-fluoro-7-(4-(2- hydroxyethyl)-1H-indol-3- yl)quinazolin-4-yl)-3,8- diazabicyclo[3.2.1]octane-8- carboxylate |
| P-0832 | 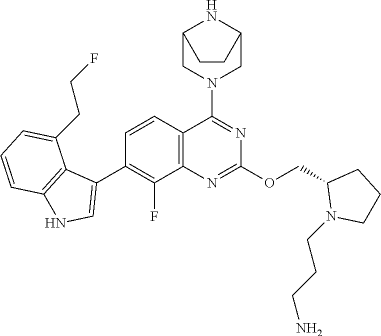 [SMILES: NCCCN1CCC[C@H]1COc1nc(N2CC3CCC(C2)N3)c2ccc(-c3c[nH]c4cccc(CCF)c34)c(F)c2n1] | 3-((S)-2-(((4-((1R,5S)-3,8- diazabicyclo[3.2.1]octan-3-yl)-8- fluoro-7-(4-(2-fluoroethyl)-1H- indol-3-yl)quinazolin-2- yl)oxy)methyl)pyrrolidin-1- yl)propan-1-amine |
| P-0833 | 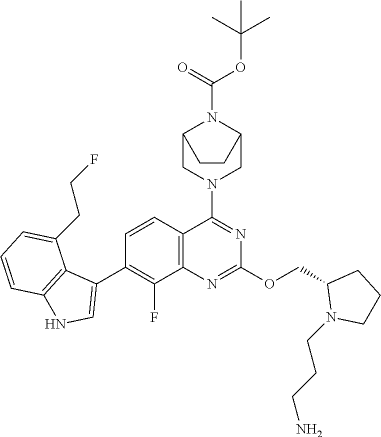 [SMILES: CC(C)(C)OC(=O)N1C2CCC1CN(c1nc(OC[C@@H]3CCCN3CCCN)nc3c(F)c(-c4c[nH]c5cccc(CCF)c45)ccc13)C2] | tert-butyl (1R,5S)-3-(2-(((S)-1-(3- aminopropyl)pyrrolidin-2- yl)methoxy)-8-fluoro-7-(4-(2- fluoroethyl)-1H-indol-3- yl)quinazolin-4-yl)-3,8- diazabicyclo[3.2.1]octane-8- carboxylate |
| P-0834 | 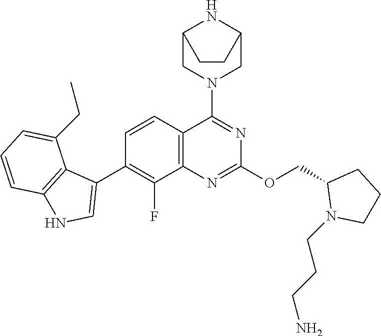 [SMILES: CCc1cccc2[nH]cc(-c3ccc4c(N5CC6CCC(C5)N6)nc(OC[C@@H]5CCCN5CCCN)nc4c3F)c12] | 3-((S)-2-(((4-((1R,5S)-3,8- diazabicyclo[3.2.1]octan-3-yl)-7-(4- ethyl-1H-indol-3-yl)-8- fluoroquinazolin-2- yl)oxy)methyl)pyrrolidin-1- yl)propan-1-amine |
| P-0835 | 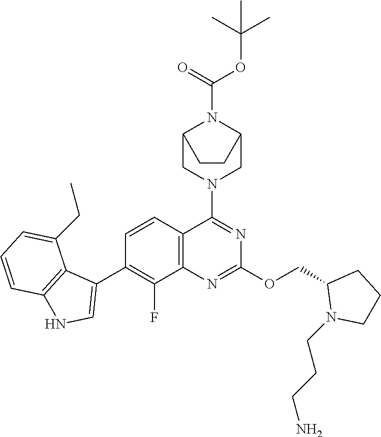 [SMILES: CCc1cccc2[nH]cc(-c3ccc4c(N5CC6CCC(C5)N6C(=O)OC(C)(C)C)nc(OC[C@@H]5CCCN5CCCN)nc4c3F)c12] | tert-butyl (1R,5S)-3-(2-(((S)-1-(3- aminopropyl)pyrrolidin-2- yl)methoxy)-7-(4-ethyl-1H-indol-3- yl)-8-fluoroquinazolin-4-yl)-3,8- diazabicyclo[3.2.1]octane-8- carboxylate |

### Biological Examples

### Biological Test Methods

The compounds of disclosure were tested using the following assays:

### KRAS Binding Disruption Assay

Compound binding to G12D or G12C-mutant KRAS was indirectly measured in a proximity-based binding disruption assays using AlphaScreen technology from Perkin Elmer. Binding of Flag-tagged G12D or G12C-mutant KRAS to biotinylated KRpep-2d analogues or to biotinylated RAF1-RBD was detected using the AlphaScreen FLAG (M2) kit. Compound binding disrupts the interaction of KRpep-2d or RAF1-RBD with KRAS, leading to a decrease in Alpha signal.

G12D or G12C-mutant human KRAS (amino acid residues 1-188) with FLAG tag and human RAF1-RBD (amino acid residues 51-131) with Avi-Tag were purified from E. coli. All assay components were prepared in 20 mM HEPES (pH 7.5), 100 mM NaCl, 5 mM MgCl2, 0.01% Tween-20, and 0.01% BSA. Biotinylated analogues of KRpep-2d were custom synthesized. The binding disruption assays were performed using 5-20 nM of mutant KRAS and 5-20 nM of either biotinylated KRpep-2d analogue or RAF1-RBD. 19 μL of KRAS protein and 4 μL of biotinylated KRpep-2d or RAF1-RBD were added to the wells of a 384-well plate containing 1 μL of various concentrations of test compound or DMSO vehicle. 16 wells containing KRAS, biotinylated KRpep-2d or RAF1-RBD, and 5% DMSO served as high controls. 16 wells containing KRAS only and 5% DMSO served as low controls. Binding reactions were incubated for 1 hour at 25° C., then Anti-Flag Acceptor and Streptavidin Donor Alpha beads were added at a final concentration of 10 μg/mL for an additional hour at room temperature. Alpha signal was read out on a Perkin Elmer Envision HTS instrument. The percentage inhibition at individual compound concentrations relative to high and low controls was calculated. The data were analyzed by using nonlinear regression to generate IC₅₀ values. Various compounds in Table IA were tested using this assay and were found to be active.

### KRAS Binding Competition Assay

Compound binding to G12V-mutant KRAS was indirectly measured in a proximity-based competition assay using AlphaScreen technology from Perkin Elmer. Binding of Flag-tagged G12V-mutant KRAS to biotinylated KRpep-2d analogues was detected using the AlphaScreen FLAG (M2) kit. Compound binding disrupts the interaction of KRpep-2d with KRAS, leading to a decrease in Alpha signal.

G12V-mutant human KRAS (amino acid residues 1-188) with FLAG tag was purified from E. coli. All assay components were prepared in 20 mM HEPES (pH 7.5), 100 mM NaCl, 5 mM MgCl2, 0.01% Tween-20, and 0.01% BSA. Biotinylated analogues of KRpep-2d were custom synthesized. The binding competition assays were performed using 10 nM of mutant KRAS and 10 nM of biotinylated KRpep-2d analogue. 19 μL of KRAS protein and 4 μL of biotinylated KRpep-2d were added to the wells of a 384-well plate containing 1 μL of various concentrations of test compound or DMSO vehicle. 16 wells containing KRAS, biotinylated KRpep-2d, and 5% DMSO served as high controls. 16 wells containing KRAS only and 5% DMSO served as low controls. Binding reactions were incubated for 1 hour at 25° C., then Anti-Flag Acceptor and Streptavidin Donor Alpha beads were added at a final concentration of 10 μg/mL for an additional hour at room temperature. Alpha signal was read out on a Perkin Elmer Envision HTS instrument. The percentage inhibition at individual compound concentrations relative to high and low controls was calculated. The data were analyzed by using nonlinear regression to generate IC₅₀ values. Various compounds in Table IA were tested using this assay and were found to be active.

### Phospho-ERK and Growth Inhibition in AGS KRASG12D Cells

Cellular activity of inhibitors was assessed in the AGS cell line that is heterozygous for KRASG12D. AGS cells were maintained and assayed in F-12K medium supplemented with 10% fetal bovine serum and 1% penicillin/streptomycin at 37° C. in a humidified incubator supplied with 5% CO₂. Phospho-ERK levels were detected using AlphaScreen technology from Perkin Elmer. The phospho-ERK assays were performed as follows. Cells were seeded in a 96-well plate in 50 μL of culture media at a density of 5×10⁴ cells per well. Compound at a maximal concentration of 5 mM was serially diluted 1:3 in DMSO for an 8-point titration. A 1 μL aliquot of each dilution point was added to 249 μL culture media. 50 μL of diluted compound was added to each well, providing 10 μM compound at the maximum concentration point. 4 wells containing cells treated with 0.2% DMSO served as high controls, and 4 wells containing cells treated with 1 μM PD0325901, a MEK inhibitor, served as low controls. After 2 hours of incubation, media was removed from the wells. Cells were lysed in 50 μL of AlphaLISA lysis buffer supplemented with protease and phosphatase inhibitors. The lysates were then transferred to a 384-well plate. Anti-mouse IgG Acceptor Alpha beads were added for a final concentration of 10 μg/mL, and anti-phospho-ERK and biotinylated anti-ERK antibodies were added for a final concentration of 0.02 nM and 0.625 nM, respectively, for 4 hours at room temperature. After the antibody and Acceptor bead incubation, Streptavidin Donor Alpha beads were added for a final concentration of 10 μg/mL for an additional 2 hours at room temperature. Alpha signal was read out on a Perkin Elmer Envision HTS instrument. The percentage inhibition at individual compound concentrations relative to high and low controls was calculated. The data were analyzed by using nonlinear regression to generate IC₅₀ values. Various compounds in Table IA were tested using this assay and were found to be active.

G Growth inhibition assays were performed as follows. Cells were seeded in U-bottom, ultra-low attachment spheroid 96-well plates in 75 μL of culture media at a density of 10³ cells per well. Compound at a maximal concentration of 5 mM was serially diluted 1:3 in DMSO for an 8-point titration. A 1 μL aliquot of each dilution point was added to 249 μL culture media. 75 μL of diluted compound was added to each well, providing 10 μM compound at the maximum concentration point. 8 wells containing cells treated with 0.2% DMSO served as high controls. After 5 days of treatment, cell viability was measured using 50 μL of CellTiter-Glo® 3D from Promega. Following incubation of the plate at room temperature for 10 minutes, luminescent signal was read on a Tecan plate reader. The percentage inhibition at individual concentrations relative to high controls was calculated. The data were analyzed by using nonlinear regression to generate IC₅₀ values. Various compounds in Table IA were tested using this assay and were found to be active.

The following Table 2 provides data, where available, indicating biochemical and/or cell inhibitory activity for exemplary compounds as described herein in Table IA. In Table 2 below, activity is provided as follows: +++=0.0001 μM<IC₅₀≤10 μM; ++=10 μM<IC₅₀≤100 μM; +=100 μM<IC₅₀≤1000 μM. Blank cells of Table 2 and compounds not listed in Table 2 indicate test results that are not yet available, and do not indicate any level of activity or potency.

TABLE 2

| | KRAS | KRAS | KRAS |
|---|---|---|---|
| | GMean | GMean | GMean |
| | IC₅₀ (uM) | IC₅₀ (uM) | IC₅₀ (uM) |
| | MUTATION: | MUTATION: | MUTATION: |
| P # | G12C | G12D | G12V |
| P-0001 | +++ | +++ | ++ |
| P-0002 | +++ | +++ | +++ |
| P-0003 | | +++ | +++ |
| P-0004 | | +++ | |
| P-0005 | +++ | +++ | ++ |
| P-0006 | ++ | +++ | |
| P-0007 | +++ | +++ | +++ |
| P-0008 | +++ | +++ | +++ |
| P-0009 | +++ | +++ | +++ |
| P-0010 | +++ | +++ | +++ |
| P-0011 | | +++ | |
| P-0012 | | +++ | |
| P-0013 | +++ | +++ | |
| P-0014 | | +++ | |
| P-0015 | +++ | +++ | +++ |
| P-0016 | | +++ | ++ |
| P-0017 | ++ | +++ | ++ |
| P-0018 | | +++ | |
| P-0019 | | ++ | ++ |
| P-0020 | +++ | +++ | ++ |
| P-0021 | +++ | +++ | +++ |
| P-0022 | ++ | +++ | |
| P-0023 | ++ | +++ | |
| P-0024 | ++ | +++ | +++ |
| P-0025 | ++ | ++ | |
| P-0026 | +++ | +++ | +++ |
| P-0027 | ++ | +++ | ++ |
| P-0028 | | +++ | + |
| P-0029 | | +++ | +++ |
| P-0030 | ++ | +++ | ++ |
| P-0031 | | +++ | ++ |
| P-0032 | ++ | +++ | ++ |
| P-0033 | | +++ | |
| P-0034 | +++ | +++ | +++ |
| P-0035 | | +++ | |
| P-0036 | +++ | +++ | |
| P-0037 | +++ | +++ | +++ |
| P-0038 | ++ | +++ | ++ |
| P-0039 | +++ | +++ | |
| P-0040 | | +++ | +++ |
| P-0041 | | +++ | |
| P-0042 | | +++ | ++ |
| P-0043 | | +++ | ++ |
| P-0044 | +++ | +++ | +++ |
| P-0045 | | +++ | |
| P-0046 | | +++ | ++ |
| P-0047 | | +++ | +++ |
| P-0048 | +++ | +++ | +++ |
| P-0049 | ++ | +++ | + |
| P-0050 | | +++ | ++ |
| P-0051 | | +++ | |
| P-0052 | | +++ | |
| P-0053 | ++ | +++ | ++ |
| P-0054 | | +++ | ++ |
| P-0055 | +++ | +++ | +++ |
| P-0056 | +++ | +++ | +++ |
| P-0057 | | ++ | |
| P-0058 | ++ | ++ | |
| P-0059 | ++ | ++ | |
| P-0060 | +++ | +++ | +++ |
| P-0061 | + | +++ | |
| P-0062 | ++ | +++ | |
| P-0063 | +++ | +++ | +++ |
| P-0064 | +++ | +++ | +++ |
| P-0065 | +++ | ++ | +++ |
| P-0066 | +++ | +++ | +++ |
| P-0067 | +++ | +++ | +++ |
| P-0068 | +++ | +++ | +++ |
| P-0069 | | +++ | |
| P-0070 | | ++ | |
| P-0071 | | +++ | |
| P-0072 | | +++ | +++ |
| P-0073 | | +++ | +++ |
| P-0074 | | +++ | |
| P-0075 | | +++ | +++ |
| P-0076 | | +++ | |
| P-0077 | | +++ | ++ |
| P-0078 | | +++ | ++ |
| P-0079 | | +++ | ++ |
| P-0080 | | +++ | ++ |
| P-0081 | | +++ | |
| P-0082 | | +++ | |
| P-0083 | | ++ | |
| P-0084 | | +++ | |
| P-0085 | +++ | +++ | +++ |
| P-0086 | | +++ | |
| P-0087 | | +++ | +++ |
| P-0088 | +++ | +++ | +++ |
| P-0089 | | +++ | +++ |
| P-0090 | | +++ | +++ |
| P-0091 | | +++ | +++ |
| P-0092 | | +++ | +++ |
| P-0093 | | +++ | +++ |
| P-0094 | | +++ | +++ |
| P-0095 | | +++ | ++ |
| P-0096 | +++ | +++ | ++ |
| P-0097 | | +++ | +++ |
| P-0098 | | +++ | +++ |
| P-0099 | +++ | +++ | +++ |
| P-0100 | +++ | +++ | +++ |
| P-0101 | | +++ | ++ |
| P-0102 | +++ | +++ | +++ |
| P-0103 | +++ | +++ | +++ |
| P-0104 | | +++ | +++ |
| P-0105 | | +++ | +++ |
| P-0106 | +++ | +++ | +++ |
| P-0107 | | +++ | +++ |
| P-0108 | | +++ | +++ |
| P-0109 | | +++ | ++ |
| P-0110 | +++ | +++ | +++ |
| P-0111 | | +++ | +++ |
| P-0112 | | +++ | +++ |
| P-0113 | | ++ | |
| P-0114 | +++ | +++ | |
| P-0115 | +++ | +++ | ++ |
| P-0116 | ++ | +++ | ++ |
| P-0117 | +++ | +++ | +++ |
| P-0118 | +++ | +++ | ++ |
| P-0119 | +++ | +++ | ++ |
| P-0120 | +++ | +++ | +++ |
| P-0121 | ++ | +++ | ++ |
| P-0122 | +++ | +++ | +++ |
| P-0123 | +++ | +++ | +++ |
| P-0124 | +++ | +++ | +++ |
| P-0125 | +++ | +++ | +++ |
| P-0126 | +++ | +++ | +++ |
| P-0127 | +++ | +++ | +++ |
| P-0128 | +++ | +++ | +++ |
| P-0129 | +++ | +++ | +++ |
| P-0130 | +++ | +++ | +++ |
| P-0131 | +++ | +++ | +++ |
| P-0132 | +++ | +++ | +++ |
| P-0133 | +++ | +++ | +++ |
| P-0134 | +++ | +++ | +++ |
| P-0135 | +++ | +++ | +++ |
| P-0136 | +++ | +++ | +++ |
| P-0137 | +++ | +++ | +++ |
| P-0138 | | +++ | +++ |
| P-0139 | | ++ | ++ |
| P-0140 | | +++ | +++ |
| P-0141 | | +++ | +++ |
| P-0142 | | +++ | +++ |
| P-0143 | | +++ | +++ |
| P-0144 | | +++ | +++ |
| P-0145 | | ++ | ++ |
| P-0146 | | +++ | +++ |
| P-0147 | | +++ | +++ |
| P-0148 | | + | ++ |
| P-0149 | | +++ | +++ |
| P-0150 | | +++ | +++ |
| P-0151 | | + | + |
| P-0152 | | ++ | ++ |
| P-0153 | | ++ | + |
| P-0154 | | ++ | ++ |
| P-0155 | | + | ++ |
| P-0156 | | + | + |
| P-0157 | | ++ | + |
| P-0158 | | ++ | ++ |
| P-0159 | | + | + |
| P-0160 | | + | + |
| P-0161 | | +++ | + |
| P-0162 | | + | + |
| P-0163 | | + | + |
| P-0164 | | ++ | + |
| P-0165 | | ++ | ++ |
| P-0166 | | + | + |
| P-0167 | | + | + |
| P-0168 | | + | ++ |
| P-0169 | | + | + |
| P-0170 | | + | + |
| P-0171 | | + | + |
| P-0172 | | ++ | + |
| P-0173 | | ++ | ++ |
| P-0174 | | + | ++ |
| P-0175 | | + | ++ |
| P-0176 | | + | + |
| P-0177 | | ++ | ++ |
| P-0178 | | + | + |
| P-0179 | | ++ | + |
| P-0180 | | ++ | + |
| P-0181 | | ++ | ++ |
| P-0182 | | + | + |
| P-0183 | | + | ++ |
| P-0184 | | ++ | + |
| P-0185 | | +++ | ++ |
| P-0186 | +++ | +++ | +++ |
| P-0187 | | +++ | +++ |
| P-0188 | | +++ | +++ |
| P-0189 | | ++ | ++ |
| P-0190 | | ++ | ++ |
| P-0191 | | ++ | ++ |
| P-0192 | | ++ | ++ |
| P-0193 | | ++ | ++ |
| P-0194 | | ++ | ++ |
| P-0195 | | ++ | ++ |
| P-0196 | | ++ | ++ |
| P-0197 | | +++ | ++ |
| P-0198 | | +++ | ++ |
| P-0199 | | ++ | ++ |
| P-0200 | | +++ | ++ |
| P-0201 | | +++ | ++ |
| P-0202 | | +++ | ++ |
| P-0203 | | +++ | +++ |
| P-0204 | | ++ | ++ |
| P-0205 | | ++ | + |
| P-0206 | | ++ | ++ |
| P-0207 | | ++ | ++ |
| P-0208 | | +++ | ++ |
| P-0209 | | ++ | ++ |
| P-0210 | | ++ | ++ |
| P-0211 | | ++ | ++ |
| P-0212 | | ++ | ++ |
| P-0213 | | ++ | ++ |
| P-0214 | | ++ | ++ |
| P-0215 | | ++ | + |
| P-0216 | | ++ | ++ |
| P-0217 | | ++ | ++ |
| P-0218 | | ++ | ++ |
| P-0219 | | ++ | ++ |
| P-0220 | | ++ | + |
| P-0221 | | ++ | ++ |
| P-0222 | | ++ | + |
| P-0223 | | ++ | ++ |
| P-0224 | | ++ | +++ |
| P-0225 | | ++ | ++ |
| P-0226 | | + | + |
| P-0227 | | ++ | + |
| P-0228 | | ++ | + |
| P-0229 | | ++ | ++ |
| P-0230 | | ++ | ++ |
| P-0231 | | ++ | ++ |
| P-0232 | | +++ | ++ |
| P-0233 | | +++ | +++ |
| P-0234 | | ++ | + |
| P-0235 | | ++ | ++ |
| P-0236 | | ++ | ++ |
| P-0237 | | ++ | ++ |
| P-0238 | | +++ | +++ |
| P-0239 | | +++ | +++ |
| P-0240 | | +++ | +++ |
| P-0241 | | +++ | +++ |
| P-0242 | | +++ | +++ |
| P-0243 | | +++ | +++ |
| P-0244 | | ++ | ++ |
| P-0245 | | +++ | +++ |
| P-0246 | | +++ | ++ |
| P-0247 | | ++ | ++ |
| P-0248 | | ++ | +++ |
| P-0249 | +++ | +++ | +++ |
| P-0250 | | +++ | +++ |
| P-0251 | | +++ | +++ |
| P-0252 | | ++ | ++ |
| P-0253 | | ++ | + |
| P-0254 | | ++ | ++ |
| P-0255 | | ++ | ++ |
| P-0256 | | ++ | ++ |
| P-0257 | | ++ | ++ |
| P-0258 | | ++ | ++ |
| P-0259 | | +++ | ++ |
| P-0260 | | ++ | ++ |
| P-0261 | | + | + |
| P-0262 | | ++ | ++ |
| P-0263 | | ++ | ++ |
| P-0264 | | ++ | ++ |
| P-0265 | | +++ | +++ |
| P-0266 | | ++ | ++ |
| P-0267 | | ++ | ++ |
| P-0268 | | ++ | ++ |
| P-0269 | | +++ | ++ |
| P-0270 | | +++ | ++ |
| P-0271 | | ++ | +++ |
| P-0272 | | +++ | ++ |
| P-0273 | | ++ | ++ |
| P-0274 | | +++ | + |
| P-0275 | | ++ | ++ |
| P-0276 | | ++ | ++ |
| P-0277 | | +++ | ++ |
| P-0278 | | +++ | ++ |
| P-0279 | +++ | +++ | +++ |
| P-0280 | | +++ | +++ |
| P-0281 | | +++ | +++ |
| P-0282 | | +++ | +++ |
| P-0283 | | ++ | ++ |
| P-0284 | | +++ | +++ |
| P-0285 | | + | + |
| P-0286 | | ++ | ++ |
| P-0287 | | +++ | +++ |
| P-0288 | | ++ | ++ |
| P-0289 | | ++ | ++ |
| P-0290 | | ++ | ++ |
| P-0291 | | ++ | ++ |
| P-0292 | | ++ | ++ |
| P-0293 | | +++ | ++ |
| P-0294 | | ++ | ++ |
| P-0295 | | +++ | + |
| P-0296 | | +++ | ++ |
| P-0297 | | +++ | +++ |
| P-0298 | | +++ | +++ |
| P-0299 | +++ | +++ | +++ |
| P-0300 | | ++ | ++ |
| P-0301 | | +++ | +++ |
| P-0302 | | +++ | ++ |
| P-0303 | | +++ | ++ |
| P-0304 | | ++ | ++ |
| P-0305 | | +++ | ++ |
| P-0306 | | +++ | ++ |
| P-0307 | | +++ | ++ |
| P-0308 | | +++ | ++ |
| P-0309 | | ++ | ++ |
| P-0310 | | +++ | ++ |
| P-0311 | | ++ | ++ |
| P-0312 | | ++ | ++ |
| P-0313 | | ++ | + |
| P-0314 | | ++ | + |
| P-0315 | | ++ | ++ |
| P-0316 | | ++ | ++ |
| P-0317 | | ++ | ++ |
| P-0318 | | +++ | ++ |
| P-0319 | | +++ | ++ |
| P-0320 | | +++ | +++ |
| P-0321 | | ++ | ++ |
| P-0322 | | ++ | ++ |
| P-0323 | | +++ | ++ |
| P-0324 | | + | ++ |
| P-0325 | | ++ | +++ |
| P-0326 | | +++ | +++ |
| P-0327 | | +++ | ++ |
| P-0328 | +++ | +++ | +++ |
| P-0329 | | +++ | ++ |
| P-0330 | | +++ | ++ |
| P-0331 | | +++ | ++ |
| P-0332 | | ++ | ++ |
| P-0333 | | +++ | ++ |
| P-0334 | | ++ | ++ |
| P-0335 | | ++ | ++ |
| P-0336 | | +++ | ++ |
| P-0337 | | +++ | + |
| P-0338 | | +++ | ++ |
| P-0339 | | ++ | ++ |
| P-0340 | | +++ | ++ |
| P-0341 | | + | ++ |
| P-0342 | | +++ | ++ |
| P-0343 | | ++ | ++ |
| P-0344 | | +++ | + |
| P-0345 | | +++ | ++ |
| P-0346 | | +++ | ++ |
| P-0347 | | +++ | +++ |
| P-0348 | | ++ | ++ |
| P-0349 | | ++ | +++ |
| P-0350 | | ++ | +++ |
| P-0351 | +++ | +++ | +++ |
| P-0352 | +++ | +++ | +++ |
| P-0353 | | +++ | +++ |
| P-0354 | +++ | +++ | +++ |
| P-0355 | | +++ | +++ |
| P-0356 | +++ | +++ | +++ |
| P-0357 | | +++ | +++ |
| P-0358 | | +++ | +++ |
| P-0360 | | +++ | ++ |
| P-0361 | | +++ | +++ |
| P-0362 | | +++ | +++ |
| P-0363 | | +++ | +++ |
| P-0364 | | +++ | +++ |
| P-0365 | +++ | +++ | +++ |
| P-0366 | +++ | +++ | +++ |
| P-0368 | | +++ | +++ |
| P-0369 | | +++ | ++ |
| P-0370 | | +++ | +++ |
| P-0371 | | ++ | ++ |
| P-0372 | | +++ | ++ |
| P-0373 | | +++ | ++ |
| P-0374 | | +++ | +++ |
| P-0375 | +++ | +++ | +++ |
| P-0376 | | +++ | +++ |
| P-0377 | | +++ | +++ |
| P-0378 | | +++ | ++ |
| P-0379 | | ++ | + |
| P-0380 | | +++ | ++ |
| P-0381 | | +++ | + |
| P-0382 | | +++ | +++ |
| P-0383 | | ++ | +++ |
| P-0384 | +++ | +++ | +++ |
| P-0385 | | +++ | ++ |
| P-0386 | | +++ | +++ |
| P-0387 | +++ | +++ | +++ |
| P-0388 | | +++ | +++ |
| P-0389 | | +++ | ++ |
| P-0390 | | +++ | +++ |
| P-0391 | | +++ | +++ |
| P-0392 | | +++ | ++ |
| P-0393 | | +++ | +++ |
| P-0394 | | +++ | +++ |
| P-0395 | | +++ | +++ |
| P-0396 | | +++ | +++ |
| P-0397 | +++ | +++ | +++ |
| P-0398 | | +++ | +++ |
| P-0399 | | +++ | +++ |
| P-0400 | | +++ | +++ |
| P-0401 | | +++ | +++ |
| P-0402 | | +++ | +++ |
| P-0403 | | +++ | ++ |
| P-0404 | | +++ | ++ |
| P-0405 | | +++ | ++ |
| P-0406 | | ++ | + |
| P-0407 | | +++ | +++ |
| P-0408 | +++ | +++ | +++ |
| P-0409 | +++ | +++ | +++ |
| P-0410 | | +++ | +++ |
| P-0411 | +++ | +++ | +++ |
| P-0412 | +++ | +++ | +++ |
| P-0413 | | +++ | ++ |
| P-0414 | +++ | +++ | +++ |
| P-0415 | +++ | +++ | +++ |
| P-0416 | | ++ | ++ |
| P-0417 | | +++ | +++ |
| P-0418 | | +++ | ++ |
| P-0419 | | +++ | ++ |
| P-0420 | | +++ | ++ |
| P-0421 | | +++ | ++ |
| P-0422 | | +++ | +++ |
| P-0423 | | +++ | +++ |
| P-0424 | | +++ | ++ |
| P-0425 | | +++ | ++ |
| P-0426 | | +++ | ++ |
| P-0427 | | ++ | + |
| P-0428 | | +++ | + |
| P-0429 | | +++ | ++ |
| P-0430 | | ++ | + |
| P-0431 | | +++ | ++ |
| P-0432 | | +++ | +++ |
| P-0433 | | +++ | +++ |
| P-0434 | | +++ | ++ |
| P-0435 | | +++ | ++ |
| P-0436 | | +++ | + |
| P-0437 | | ++ | + |
| P-0438 | | ++ | ++ |
| P-0439 | | +++ | ++ |
| P-0440 | | +++ | ++ |
| P-0441 | | +++ | +++ |
| P-0442 | | ++ | +++ |
| P-0443 | +++ | +++ | +++ |
| P-0444 | | ++ | + |
| P-0445 | | ++ | ++ |
| P-0446 | | ++ | ++ |
| P-0447 | | +++ | +++ |
| P-0448 | | +++ | ++ |
| P-0449 | | +++ | +++ |
| P-0450 | | ++ | ++ |
| P-0451 | | +++ | ++ |
| P-0452 | | +++ | +++ |
| P-0453 | | +++ | + |
| P-0454 | | +++ | + |
| P-0455 | | +++ | ++ |
| P-0456 | | ++ | ++ |
| P-0457 | | ++ | ++ |
| P-0458 | | ++ | + |
| P-0459 | | +++ | +++ |
| P-0460 | | ++ | ++ |
| P-0461 | | +++ | +++ |
| P-0462 | | ++ | ++ |
| P-0463 | +++ | +++ | +++ |
| P-0464 | | ++ | +++ |
| P-0465 | | +++ | ++ |
| P-0466 | | +++ | ++ |
| P-0467 | | +++ | ++ |
| P-0468 | | +++ | ++ |
| P-0469 | | +++ | + |
| P-0470 | | + | ++ |
| P-0471 | | +++ | + |
| P-0472 | | +++ | +++ |
| P-0473 | | + | + |
| P-0474 | | +++ | ++ |
| P-0475 | | +++ | ++ |
| P-0502 | +++ | +++ | +++ |
| P-0504 | +++ | +++ | +++ |
| P-0505 | +++ | +++ | |
| P-0506 | | +++ | ++ |
| P-0507 | | +++ | |
| P-0508 | | +++ | |
| P-0509 | | +++ | |
| P-0510 | +++ | +++ | +++ |
| P-0511 | | +++ | +++ |
| P-0512 | | +++ | |
| P-0513 | | +++ | |
| P-0514 | | +++ | |
| P-0515 | | +++ | |
| P-0516 | | +++ | |
| P-0517 | | +++ | |
| P-0518 | | +++ | |
| P-0519 | | +++ | |
| P-0520 | | +++ | |
| P-0521 | | +++ | |
| P-0522 | | +++ | |
| P-0523 | | +++ | |
| P-0524 | | +++ | |
| P-0525 | | +++ | |
| P-0526 | | +++ | |
| P-0527 | | +++ | |
| P-0528 | | +++ | |
| P-0529 | | +++ | |
| P-0530 | | +++ | |
| P-0531 | | +++ | |
| P-0532 | | +++ | |
| P-0533 | | +++ | |
| P-0534 | | +++ | |
| P-0535 | | +++ | |
| P-0536 | | +++ | |
| P-0537 | | +++ | |
| P-0538 | | +++ | |
| P-0539 | | +++ | |
| P-0540 | | +++ | |
| P-0541 | | +++ | |
| P-0542 | | +++ | |
| P-0543 | | +++ | |
| P-0544 | | +++ | |
| P-0545 | | +++ | |
| P-0546 | | +++ | |
| P-0547 | | +++ | |
| P-0548 | | +++ | |
| P-0549 | | +++ | |
| P-0550 | | +++ | |
| P-0551 | | +++ | |
| P-0552 | | +++ | |
| P-0553 | | +++ | |
| P-0554 | | +++ | |
| P-0555 | | +++ | |
| P-0556 | | +++ | |
| P-0557 | | +++ | |
| P-0558 | | +++ | |
| P-0559 | | +++ | |
| P-0560 | | +++ | |
| P-0561 | | +++ | |
| P-0562 | | ++ | |
| P-0563 | | +++ | |
| P-0564 | | +++ | |
| P-0565 | | +++ | |
| P-0566 | | +++ | |
| P-0567 | | +++ | |
| P-0568 | | ++ | |
| P-0569 | | +++ | |
| P-0570 | | +++ | |
| P-0571 | | +++ | |
| P-0572 | | +++ | |
| P-0573 | | +++ | |
| P-0574 | | +++ | |
| P-0575 | | +++ | |
| P-0576 | | +++ | |
| P-0577 | | +++ | |
| P-0578 | | +++ | |
| P-0579 | | +++ | |
| P-0580 | | +++ | |
| P-0581 | | +++ | |
| P-0582 | | +++ | |
| P-0583 | | +++ | |
| P-0584 | | +++ | |
| P-0585 | | +++ | |
| P-0586 | | +++ | |
| P-0587 | | +++ | |
| P-0588 | | +++ | |
| P-0589 | | +++ | |
| P-0590 | | +++ | |
| P-0591 | | +++ | |
| P-0592 | | +++ | |
| P-0593 | | +++ | |
| P-0594 | | +++ | |
| P-0595 | | +++ | |
| P-0596 | | +++ | |
| P-0598 | | +++ | |
| P-0599 | | +++ | |
| P-0600 | | +++ | |
| P-0601 | | +++ | |
| P-0602 | | +++ | |
| P-0604 | | +++ | |
| P-0605 | | ++ | |
| P-0606 | | +++ | |
| P-0607 | | +++ | |
| P-0608 | | +++ | |
| P-0609 | | ++ | + |
| P-0610 | | +++ | + |
| P-0611 | | ++ | + |
| P-0612 | | +++ | ++ |
| P-0613 | | +++ | ++ |
| P-0614 | | +++ | ++ |
| P-0615 | | +++ | ++ |
| P-0616 | | +++ | ++ |
| P-0617 | | +++ | ++ |
| P-0618 | | +++ | ++ |
| P-0619 | | +++ | ++ |
| P-0620 | | +++ | ++ |
| P-0621 | | +++ | ++ |
| P-0622 | | +++ | ++ |
| P-0623 | | +++ | ++ |
| P-0624 | | +++ | ++ |
| P-0625 | | +++ | +++ |
| P-0626 | | +++ | ++ |
| P-0627 | | +++ | +++ |
| P-0628 | | +++ | +++ |
| P-0629 | | +++ | ++ |
| P-0630 | | +++ | ++ |
| P-0631 | | +++ | ++ |
| P-0632 | | +++ | +++ |
| P-0633 | | +++ | + |
| P-0634 | | +++ | + |
| P-0635 | | +++ | ++ |
| P-0636 | +++ | +++ | |
| P-0637 | ++ | +++ | |
| P-0638 | ++ | +++ | |
| P-0639 | ++ | +++ | |
| P-0640 | ++ | +++ | |
| P-0641 | ++ | +++ | |
| P-0642 | +++ | +++ | |
| P-0643 | ++ | +++ | |
| P-0645 | ++ | +++ | |
| P-0646 | +++ | +++ | |
| P-0647 | ++ | +++ | |
| P-0648 | ++ | +++ | |
| P-0649 | +++ | +++ | |
| P-0650 | +++ | +++ | |
| P-0651 | +++ | +++ | |
| P-0652 | ++ | +++ | |
| P-0653 | +++ | +++ | |
| P-0654 | ++ | +++ | |
| P-0655 | + | +++ | |
| P-0656 | ++ | +++ | |
| P-0658 | + | +++ | |
| P-0659 | +++ | +++ | |
| P-0660 | +++ | +++ | |
| P-0661 | +++ | +++ | |
| P-0662 | +++ | +++ | |
| P-0663 | +++ | +++ | |
| P-0665 | ++ | ++ | |
| P-0666 | +++ | +++ | |
| P-0667 | +++ | +++ | |
| P-0668 | +++ | +++ | |
| P-0669 | +++ | +++ | |
| P-0670 | + | +++ | |
| P-0671 | ++ | +++ | |
| P-0672 | ++ | +++ | |
| P-0673 | ++ | +++ | |
| P-0674 | +++ | +++ | |
| P-0675 | +++ | +++ | |
| P-0676 | ++ | +++ | |
| P-0677 | ++ | +++ | |
| P-0678 | +++ | +++ | |
| P-0679 | +++ | +++ | |
| P-0680 | + | +++ | |
| P-0681 | + | +++ | |
| P-0682 | ++ | +++ | |
| P-0683 | + | +++ | |
| P-0684 | + | +++ | |
| P-0685 | ++ | +++ | |
| P-0686 | + | +++ | |
| P-0687 | + | +++ | |
| P-0688 | ++ | +++ | |
| P-0689 | ++ | +++ | |
| P-0690 | ++ | +++ | |
| P-0691 | ++ | +++ | |
| P-0692 | ++ | +++ | |
| P-0693 | ++ | +++ | |
| P-0694 | ++ | +++ | |
| P-0695 | ++ | +++ | |
| P-0696 | ++ | +++ | |
| P-0697 | +++ | +++ | |
| P-0698 | ++ | +++ | |
| P-0699 | +++ | +++ | |
| P-0700 | ++ | +++ | |
| P-0701 | + | +++ | |
| P-0702 | ++ | +++ | |
| P-0703 | ++ | +++ | |
| P-0704 | ++ | +++ | |
| P-0705 | ++ | +++ | |
| P-0706 | + | +++ | |
| P-0709 | +++ | +++ | |
| P-0710 | +++ | +++ | |
| P-0711 | +++ | +++ | |
| P-0712 | ++ | ++ | |
| P-0713 | ++ | +++ | |
| P-0714 | ++ | +++ | |
| P-0715 | ++ | ++ | |
| P-0716 | ++ | +++ | |
| P-0717 | ++ | +++ | |
| P-0718 | ++ | +++ | |
| P-0719 | +++ | +++ | |
| P-0720 | ++ | +++ | |
| P-0721 | ++ | +++ | |
| P-0722 | ++ | +++ | |
| P-0723 | +++ | +++ | |
| P-0724 | + | ++ | |
| P-0725 | + | ++ | |
| P-0726 | ++ | +++ | |
| P-0727 | + | + | |
| P-0728 | + | ++ | |
| P-0729 | ++ | +++ | |
| P-0730 | +++ | +++ | |
| P-0731 | +++ | +++ | |
| P-0732 | ++ | +++ | |
| P-0733 | ++ | +++ | |
| P-0734 | ++ | +++ | |
| P-0735 | + | +++ | |
| P-0736 | ++ | +++ | |
| P-0737 | +++ | +++ | |
| P-0738 | ++ | +++ | |
| P-0739 | ++ | +++ | |
| P-0740 | ++ | +++ | |
| P-0741 | ++ | +++ | |
| P-0742 | ++ | +++ | |
| P-0743 | ++ | +++ | |
| P-0744 | ++ | +++ | |
| P-0745 | ++ | +++ | |
| P-0746 | ++ | +++ | |
| P-0747 | ++ | +++ | |
| P-0748 | ++ | +++ | |
| P-0749 | ++ | +++ | |
| P-0750 | ++ | +++ | |
| P-0751 | ++ | +++ | |
| P-0752 | ++ | +++ | |
| P-0753 | +++ | +++ | |
| P-0754 | ++ | +++ | |
| P-0755 | ++ | +++ | |
| P-0756 | + | +++ | |
| P-0757 | + | +++ | |
| P-0758 | + | +++ | |
| P-0759 | ++ | +++ | |
| P-0760 | ++ | +++ | |
| P-0761 | + | +++ | |
| P-0762 | ++ | ++ | |
| P-0763 | +++ | +++ | |
| P-0764 | +++ | +++ | |
| P-0765 | +++ | +++ | |
| P-0766 | ++ | +++ | |
| P-0767 | ++ | +++ | |
| P-0768 | ++ | +++ | |
| P-0769 | ++ | +++ | |
| P-0770 | ++ | +++ | |
| P-0771 | ++ | +++ | |
| P-0772 | ++ | +++ | |
| P-0773 | +++ | +++ | |
| P-0774 | +++ | +++ | |
| P-0775 | ++ | +++ | |
| P-0776 | ++ | +++ | |
| P-0777 | ++ | +++ | |
| P-0778 | ++ | +++ | |
| P-0779 | ++ | +++ | |
| P-0780 | + | ++ | |
| P-0781 | + | ++ | |
| P-0782 | ++ | +++ | |
| P-0783 | + | +++ | |
| P-0784 | ++ | +++ | |
| P-0785 | + | + | |
| P-0786 | +++ | +++ | |
| P-0787 | ++ | +++ | |
| P-0788 | +++ | +++ | |
| P-0789 | ++ | +++ | |
| P-0790 | +++ | +++ | |
| P-0791 | + | +++ | |
| P-0792 | ++ | +++ | |
| P-0793 | ++ | +++ | |
| P-0794 | ++ | +++ | |
| P-0795 | ++ | ++ | |
| P-0796 | + | +++ | |
| P-0797 | ++ | +++ | |
| P-0798 | + | ++ | |
| P-0799 | + | ++ | |
| P-0800 | + | ++ | |
| P-0801 | + | ++ | |
| P-0802 | ++ | +++ | |
| P-0803 | +++ | +++ | |
| P-0804 | ++ | +++ | |
| P-0805 | ++ | +++ | |
| P-0806 | +++ | +++ | |
| P-0807 | +++ | +++ | |
| P-0808 | +++ | +++ | |
| P-0809 | ++ | +++ | |
| P-0810 | ++ | +++ | |
| P-0811 | + | +++ | |
| P-0812 | ++ | +++ | |
| P-0813 | +++ | ++ | |
| P-0818 | | +++ | |

It has been observed that the compounds of this disclosure exhibit an unexpectedly higher potency for KRAS G12D solely because of the unique structural distinction of having a bridged heterocycloalkyl at the R¹ variable. Table 3 below provides side-by-side comparisons of representative compounds of this disclosure to the non-bridged versions of these compounds at the R₁ variable.

TABLE 3

| | | KRAS GMean | Improved KRAS G12D |
|---|---|---|---|
| | | IC₅₀ (uM) | Biochemical Potency of |
| | | MUTATION: | Compounds with Bridged |
| P # | STRUCTURE | G12D | vs Non-Bridged R¹ Groups. |
| P-0027 | 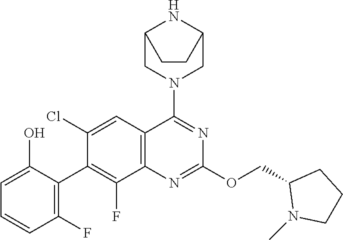 [SMILES: CN1CCC[C@H]1COc1nc(N2CC3CCC(C2)N3)c2cc(Cl)c(-c3c(O)cccc3F)c(F)c2n1] | 0.038 | |
| Non-bridged version of P-0027 | 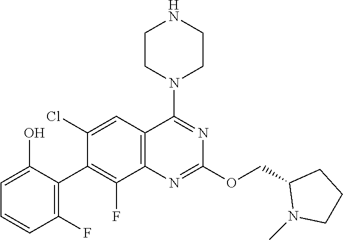 [SMILES: CN1CCC[C@H]1COc1nc(N2CCNCC2)c2cc(Cl)c(-c3c(O)cccc3F)c(F)c2n1] | 14.919 | P-0027 is about 392 times more potent than its non- bridged version at the R¹ variable. |
| P-0001 | 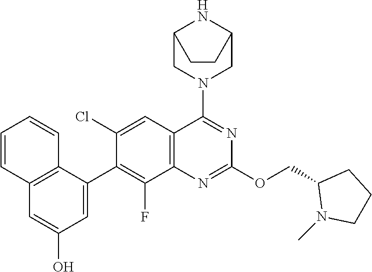 [SMILES: CN1CCC[C@H]1COc1nc(N2CC3CCC(C2)N3)c2cc(Cl)c(-c3cc(O)cc4ccccc34)c(F)c2n1] | 0.016 | |
| Non-bridged version of P-0001 | 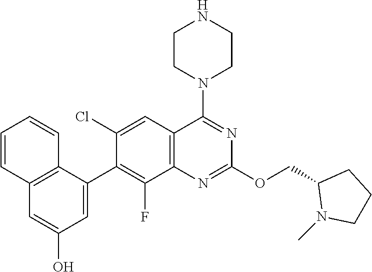 [SMILES: CN1CCC[C@H]1COc1nc(N2CCNCC2)c2cc(Cl)c(-c3cc(O)cc4ccccc34)c(F)c2n1] | 1.115 | P-0001 is about 70 times more potent than its non- bridged version at the R¹ variable. |

It has been observed that when comparing structurally similar compounds from the art, such as those listed in Table 4 from WO 2017/172979, to compounds of this disclosure, that the compounds of this disclosure having the structurally unique bridged heterocycloalkyl at the R¹ variable exhibit an unexpectedly higher potency for KRAS G12D. Table 4 below provides side-by-side comparisons of structurally similar compounds in WO 2017/172979 that do not contain such a bridge to compounds in this disclosure that do contain this bridge.

TABLE 4

| | | | Improved KRAS |
|---|---|---|---|
| | | | G12D Biochemical |
| | | KRAS GMean | Potency of |
| | | IC₅₀ (uM) | Compounds with |
| Compound | | MUTATION: | Bridged vs Non- |
| Source | STRUCTURE | G12D | Bridged R¹ Groups. |
| P-0020 | 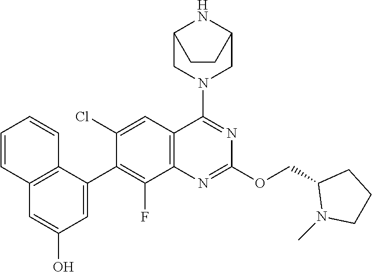 [SMILES: CN1CCC[C@H]1COc1nc(N2CC3CCC(C2)N3)c2cc(Cl)c(-c3cc(O)cc4ccccc34)c(F)c2n1] | 0.016 | |
| Compound 36 in WO 2017/172979 | 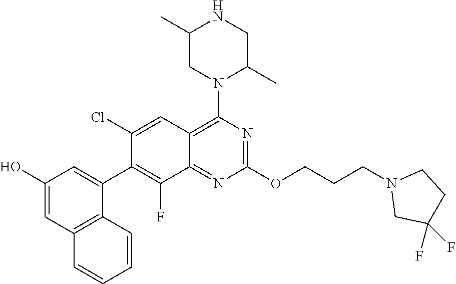 [SMILES: CC1CN(c2nc(OCCCN3CCC(F)(F)C3)nc3c(F)c(-c4cc(O)cc5ccccc45)c(Cl)cc23)C(C)CN1] | 3.065 | P-0020 is about 192 times more potent than Compound 36 in WO 2017/172979. |
| P-0023 | 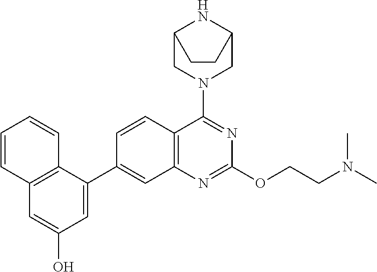 [SMILES: CN(C)CCOc1nc(N2CC3CCC(C2)N3)c2ccc(-c3cc(O)cc4ccccc34)cc2n1] | 0.29 | |
| Compound 149 in WO 2017/172979 | 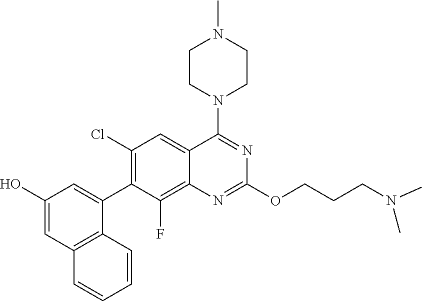 [SMILES: CN(C)CCCOc1nc(N2CCN(C)CC2)c2cc(Cl)c(-c3cc(O)cc4ccccc34)c(F)c2n1] | 16.522 | P-0023 is about 57 times more potent than Compound 149 in WO 2017/172979. |

Other embodiments of this disclosure relate to compounds that exhibit an unexpectedly higher potency for KRAS G12V solely because of the unique structural distinction of having a bridged heterocycloalkyl at the R¹ variable vs the same compounds that do not. Other embodiments of this disclosure relate to compounds that exhibit a higher potency for KRAS G12D over KRAS G12C. Other embodiments of this disclosure relate to compounds that exhibit a higher potency for KRAS G12D over KRAS G12V. Other embodiments of this disclosure relate to compounds that exhibit a higher potency for KRAS G12V over KRAS G12C. Other embodiments of this disclosure relate to compounds that exhibit a higher potency for KRAS G12V over KRAS G12D.

All patents and other references cited herein are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of the embodiments described herein are exemplary and are not intended as limitations on the scope of the disclosure. Changes therein and other uses will occur to those skilled in the art, which are encompassed within the spirit of the disclosure, are defined by the scope of the claims.

It will be readily apparent to one skilled in the art that varying substitutions and modifications may be made to the present disclosure described herein without departing from the scope and spirit of the disclosure. For example, variations can be made to provide additional compounds of the compounds of this disclosure and/or various methods of administration can be used. Thus, such additional embodiments are within the scope of the present disclosure and the following claims.

The present disclosure illustratively described herein suitably may be practiced in the absence of any element or elements, limitation or limitations which is not specifically described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically described by the embodiments and optional features, modification and variation of the concepts herein described may be resorted to by those skilled in the art, and that such modifications and variations are considered to be within the scope of this disclosure as defined by the appended claims.

In addition, where features or aspects of the disclosure are described in terms grouping of alternatives, those skilled in the art will recognize that the disclosure is also thereby described in terms of any individual member or subgroup of members of the groups described herein.

Also, unless indicated to the contrary, where various numerical values are provided for embodiments, additional embodiments are described by taking any 2 different values as the endpoints of a range. Such ranges are also within the scope of the present disclosure.

Thus, additional embodiments are within the scope of the disclosure and within the following claims.

LENGTHY TABLES
The patent application contains a lengthy table section. A copy of the table is available in electronic form from the USPTO web site (https://seqdata.uspto.gov/?pageRequest=docDetail&DocID=US20230081426A1). An electronic copy of the table will also be available from the USPTO upon request and payment of the fee set forth in 37 CFR 1.19(b)(3).

1-32. (canceled)

33. A compound having Formula F 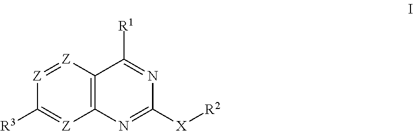
or a pharmaceutically acceptable salt, a solvate, a tautomer, a stereoisomer, or a deuterated analog thereof, wherein:
R¹ is bridged heterocycloalkyl substituted with 0-4 T¹ and 1 G;
R² is alkyl substituted with 0-5 R⁴ and 0-2 J, aryl substituted with 0-4 R⁴ and 0-1 J, arylalkyl substituted with 0-4 R⁴ and 0-1 J, heteroaryl substituted with 0-5 R⁴ and 0-1 J, heteroarylalkyl substituted with 0-5 R⁴ and 0-1 J, heterocycloalkyl substituted with 0-5 R⁴ and 0-1 J, heterocycloalkylalkyl substituted with 0-5 R⁴ and 0-1 J, bridged heterocycloalkyl substituted with 0-5 R⁴ and 0-1 J, bridged heterocycloalkylalkyl substituted with 0-5 R⁴ and 0-1 J, cycloalkyl substituted with 0-5 R⁴ and 0-1 J; cycloalkylalkyl substituted with 0-5 R⁴ and 0-1 J, or 6-11 membered heterocyclic spiro ring substituted with 0-5 R⁴ and 0-1 J; provided that when R² is alkyl substituted with 0-5 R⁴ and 0-2 J, X is not —[C(R¹⁰)₂]₁₋₆;
R³ is aryl, heteroaryl, —N(H)-heteroaryl, or heterocycloalkyl, wherein R³ is substituted with 0-4 R⁵ and 0-1 M;
each R⁴ is independently alkyl, halogen, hydroxyl, CN, haloalkyl, hydroxyalkyl, C₁-C₆cyanoalkyl, or alkoxy, provided that when R⁴ is attached to a nitrogen atom, R⁴ is not halogen or CN;
each R⁵ is independently alkyl, alkynyl, alkoxy, halogen, hydroxyl, CN, haloalkyl, C₁-C₆cyanoalkyl, C₂-C₄alkenyl, alkoxyalkyl, cycloalkyl, cycloalkylalkyl, or hydroxyalkyl, provided that when R⁵ is attached to a nitrogen atom, R⁵ is not halogen or CN;
each R⁶ is independently H, alkyl, haloalkyl, or hydroxyalkyl;
each R⁷ is independently H, halogen, alkyl, alkenyl, alkynyl, CN, haloalkyl, hydroxyalkyl, cycloalkyl, —C(O)OR¹⁰, or alkoxy optionally substituted with 1-3 halogens;
each R⁸ is independently alkyl, halogen, hydroxyl, —NH₂, —N(H)alkyl, —N(alkyl)₂, CN, SH, haloalkyl, or hydroxyalkyl, provided that when R⁸ is attached to nitrogen, R⁸ is not halogen, hydroxyl, —NH₂, —N(H)alkyl, —N(alkyl)₂, or CN;
each R⁹ is independently halogen, hydroxyl, CN, haloalkyl, or hydroxyalkyl;
each R¹⁰ is independently H, N(H)₂, alkyl, hydroxyalkyl, or haloalkyl;
R¹¹ is cycloalkyl substituted with 0-4 R⁴ and 0-1 J;
each J is independently is —N(R¹⁰)₂, —NO₂, —SO₂N(R¹⁰)₂, —NH—SO₂R¹⁰, —NHC(O)N(R¹⁰)₂, —C(O)N(R¹⁰)₂, —C(O)C(R¹⁰)₂-aryl, —C(O)OCH₂-aryl, -alkyl-N(R¹⁰)₂, -alkyl-NHC(O)alkyl, -alkyl-NHC(O)OCH₂-phenyl, —C(O)-alkylene-OH, —OC(O)R¹⁰, heterocycloalkyl substituted with 0-1 hydroxy and 0-1 halo, or —C(O)OR¹⁰; provided that when J is N(R¹⁰)₂, —NO₂, or —SO₂N(R¹⁰)₂, J is not attached to a nitrogen atom;
M is —C(O)R¹⁰, —C(O)NH₂, —N(H)C(O)CH₃, —N(H)SO₂-alkyl, NO₂, —C₁-C₃alkyl-C(O)OR¹⁰, or —N(R¹⁰)₂, provided that M is not attached to a nitrogen atom;
X is absent, —[C(R¹⁰)₂]₁₋₆—, —[C(R¹⁰)₂]₀₋₄—O—, —O—[C(R¹⁰)₂]₁₋₄—, C₂-C₆ alkenylene, —NR⁶—[C(R¹⁰)₂]₀₋₄—, or —[C(R¹⁰)₂]₀₋₄—NR⁶—;
each Z is independently CR⁷ or N;
G is L¹-L²-T² provided that:
when Formula I is Formula I′ 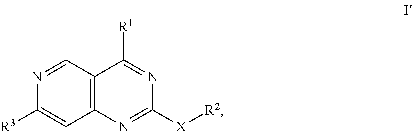
then G is not hydrogen;
each T¹ is independently alkyl, halogen, hydroxyl, haloalkyl, or hydroxyalkyl, provided that when T¹ is attached to nitrogen, T¹ is not halogen or hydroxyl;
T² is H, CN, —C(NH)N(H)CN, —C(NH)OCH₃, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)—SO₂N(R¹⁰)₂, —N(R¹⁰)—C(O)—N(R¹⁰)₂, —N(R¹⁰)—SO₂(R¹⁰), —N(R¹⁰)—C(O)O(R¹⁰), —P(O)(R¹⁰)₂, —N(R¹⁰)C(O)-alkyl, —O—C(O)-alkyl, —C(═NR¹⁰)N(R¹⁰)₂, cycloalkyl, heterocycloalkyl, bridged heterocycloalkyl, heterocyclic spiro ring, carbocyclic spiro ring, aryl, or heteroaryl, wherein T² is substituted with 0-4 R⁸ and 0-1 R¹¹ provided that when L¹ and L² are both absent, T² is not CN, —N(R¹⁰)—SO₂N(R¹⁰)₂, —N(R¹⁰)—C(O)—N(R¹⁰)₂, N(R¹⁰)₂, —OR¹⁰, —N(R¹⁰)C(O)-alkyl, or —O—C(O)-alkyl, and provided that when L¹ and L² taken together are —C(O), —C(O)C(O)N(H)—, —C(O)O—, —C(O)N(H)—, —S(O)₂—, -alkylene-C(O)N(H)—, -alkylene-C(O)—, —SO₂—, or —SO₂NH—, T² is not CN, —N(R¹⁰)—SO₂N(R¹⁰)₂, —N(R¹⁰)—C(O)—N(R¹⁰)₂, —C(O)OR¹⁰, —N(R¹⁰)₂, —OR¹⁰, —C(O)N(R¹⁰)₂, —SO₂N(R¹⁰)₂, —N(R¹⁰)C(O)-alkyl, —O—C(O)-alkyl, —C(NH)OCH₃, or —C(═NR¹⁰)N(R¹⁰)₂;
L¹ is absent, —C(O)—, —C(O)C(O)N(H)—, —C(O)O—, —C(O)N(H)—, —C(O)N(H)—O—, -alkylene-C(O)N(H)—, -alkylene-C(O)—, —SO₂—, or —SO₂N(H)—, provided that when L¹ is —C(O)N(H)—O—, T² is H; and
L² is absent, alkylene substituted with 0-4 R⁹, —C₂-C₄alkenylene, or C₃-C₆alkenylene substituted with 0-4 R⁹, provided that when L¹ is —C(O)— and L² is absent, T² is not H.

34. The compound according to claim 33, wherein:
X is absent, —[C(R¹⁰)₂]₁₋₄—, —(CH₂)₀₋₃—O—, O—[C(R¹⁰)₂]₁₋₃—, C₃-C₄ alkenylene, or —NR⁶—
R¹ is a 6-9 membered bridged heterocycloalkyl having at least one nitrogen atom, wherein R¹ is substituted with 0-4 T¹ and 1 G;
R² is C₁-G₆alkyl substituted with 0-5 R⁴ and 0-2 J, phenyl substituted with 0-4 R⁴ and 0-1 J, —C₀-C₆alkyl-phenyl substituted with 0-4 R⁴ and 0-1 J, 5-10 membered heteroaryl substituted with 0-5 R⁴ and 0-1 J, —C₀-C₆alkyl-5-10 membered heteroaryl substituted with 0-5 R⁴ and 0-1 J, —C₀-C₆alkyl-4-10 membered heterocycloalkyl substituted with 0-5 R⁴ and 0-1 J, or —C₁-C₆alkyl-C₃-C₁₀cycloalkyl substituted with 0-5 R⁴ and 0-1 J, provided that when R² is C₁-C₆alkyl substituted with 0-5 R⁴ and 0-2 J, X is not —C[(R¹⁰)₂]₁₋₃;
R³ is phenyl, naphthyl, 5-10 membered heteroaryl, or 5-10 membered heterocycloalkyl, wherein R³ is substituted with 0-4 R⁵ and 0-1 M;
each R⁴ is independently C₁-C₆alkyl, halogen, hydroxyl, CN, C₁-C₆haloalkyl, C₁-C₆hydroxyalkyl, C₁-C₆cyanoalkyl, or C₁-C₆alkoxy, provided that when R⁴ is attached to a nitrogen atom, R⁴ is not halogen, or CN;
each R⁵ is independently C₁-C₆alkyl, C₁-C₆alkynyl, C₁-C₆alkoxy, halogen, hydroxyl, CN, C₁-C₆haloalkyl, C₁-C₆cynoalkyl, or C₁-C₆hydroxyalkyl, provided that when R⁵ is attached to a nitrogen atom, R⁵ is not halogen or CN;
each R⁶ is independently H, C₁-C₆alkyl, C₁-C₆haloalkyl, or C₁-C₆hydroxyalkyl;
each R⁷ is independently H, halogen, C₁-C₆alkyl, C₂-C₄alkenyl, C₂-C₄alkynyl, CN, C₁-C₆haloalkyl, C₁-C₄hydroxyalkyl; C₃-C₄cycloalkyl, —C(O)OR¹⁰, or C₁-C₄alkoxy optionally substituted with 1-3 halogens;
each R⁸ is independently C₁-C₆alkyl, halogen, hydroxyl, —NH₂, —N(H)C₁-C₆alkyl, —N(C₁-C₆alkyl)₂, CN, C₁-C₆haloalkyl, or C₁-C₆hydroxyalkyl, provided that when R⁸ is attached to nitrogen, R⁸ is not halogen, hydroxyl, —NH₂, —N(H)C₁-C₆alkyl, —N(C₁-C₆alkyl)₂, or CN;
each R⁹ is independently halogen, hydroxyl, CN, C₁-C₆haloalkyl, or C₁-C₆hydroxyalkyl;
each R¹⁰ is independently H, —N(H)₂, C₁-C₆alkyl, or C₁-C₆1haloalkyl;
R¹¹ is C₁-C₆cycloalkyl substituted with 0-3 R⁴ and 0-1 J;
each J is independently is —N(R¹⁰)₂, —SO₂N(R¹⁰)₂, —C(O)N(R¹⁰)₂, or —C(O)OR¹⁰; provided that when J is —N(R¹⁰)₂ or —SO₂N(R¹⁰)₂, J is not attached to a nitrogen atom;
each T¹ is independently C₁-C₆alkyl, halogen, hydroxyl, C₁-C₆haloalkyl, or C₁-C₆hydroxyalkyl, provided that when T¹ is attached to nitrogen, T¹ is not halogen or hydroxyl;
T² is H, CN, —C(O)OR¹⁰, —N(R¹⁰)₂, —OR¹⁰, —C(O)N(R¹⁰)₂, —SO₂N(R¹⁰)₂, —SO₂R¹⁰, —N(R¹⁰)C(O)—C₁-C₆alkyl, —O—C(O)—C₁-C₆alkyl, —C(═NH)NH₂, C₁-C₆cycloalkyl, 4-10 membered heterocycloalkyl, 5-9 membered bridged heterocycloalkyl, 6-9 membered heterocyclic spiro ring, 6-9 membered carbocyclic spiro ring, phenyl, naphthyl, or 5-10 membered heteroaryl, wherein T² is substituted with 0-4 R⁸ and 0-1 R¹¹;
provided that when L¹ and L² are both absent, T² is not CN, —N(R¹⁰)₂, —OR¹⁰, —N(R¹⁰)C(O)—C₁-C₃alkyl, or —O—C(O)—C₁-C₃alkyl, and provided that when L¹ and L² taken together are —C(O)—, —C(O)C(O)N(H)—, —C(O)O—, —C(O)N(H)—, —C(O)N(H)—O—, —C₁-C₃alkyelene-C(O)N(H)—, —C₁-C₃alkylene-C(O)—, —SO₂—, or —SO₂NH—, T² is not CN, —C(O)OR¹⁰, C₁-C₆alkoxy, N(R¹⁰)₂, —OR¹⁰, —C(O)N(R¹⁰)₂, —SO₂N(R¹⁰)₂, —N(R¹⁰)C(O)—C₁-C₆alkyl, —O—C(O)—C₁-C₆alkyl, or —C(═NH)NH₂;
L¹ is absent, —C(O)—, —C(O)C(O)N(H)—, —C(O)O—, —C(O)N(H)—, —C₁-C₃alkyelene-C(O)N(H)—, —C₁-C₃alkylene-C(O)—, —SO₂—, or —SO₂N(H)— provided that when L¹ is —C(O)N(H)—O—, T² is H;
L² is absent, C₁-C₆alkylene substituted with 0-2 R⁹, or C₁-C₆alkenylene substituted with 0-4 R⁹, provided that when L¹ is —C(O)— and L² is absent, T² is not H; and
M is —C(O)NH₂, —NH₂C(O)CH₃, —N(H)SO₂—C₁-C₆alkyl, or —NH₂, provided that M is not attached to a nitrogen atom.

35. The compound according to claim 33 having one of the following formulae: 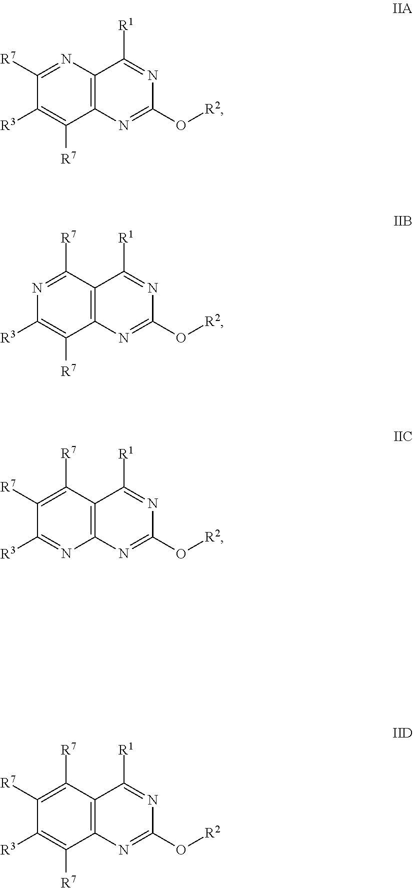
or a pharmaceutically acceptable salt, a solvate, a tautomer, a stereoisomer, or a deuterated analog thereof.

36. The compound according to claim 35, wherein G is H, provided that when the compound has the Formula (IIB), R⁷ is not H.

37. The compound according to claim 35, wherein G is not H.

38. The compound according to claim 35, wherein G is not hydrogen, alkyl, hydroxyalkyl, alkenyl, benzyl, alkoxyalkyl, alkylaminoalkyl, alkylcarbonyl, alkylcarbonylaminoalkyl, cycloalkyl, heterocycloalkyl, bridged heterocycloalkyl, heterocyclic spiro ring, aryl, or heteroaryl.

39. The compound according to claim 35, wherein G has one of the following Formulae:

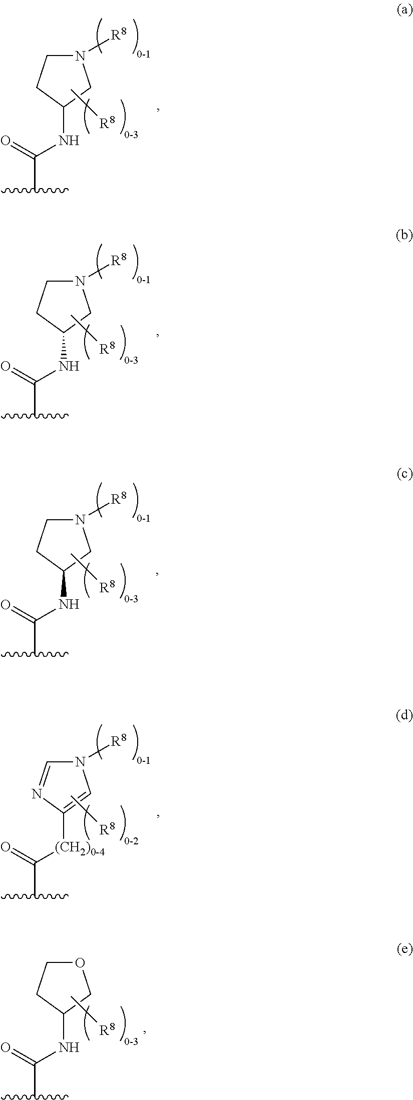

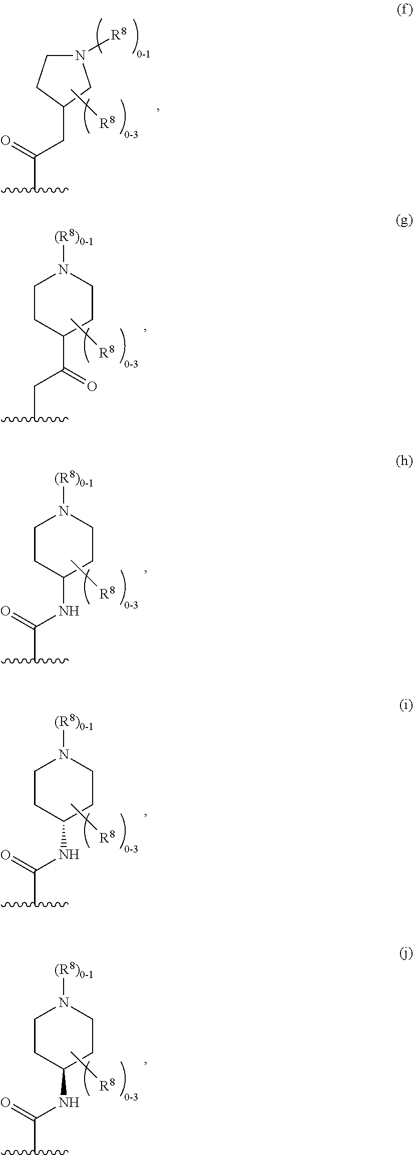

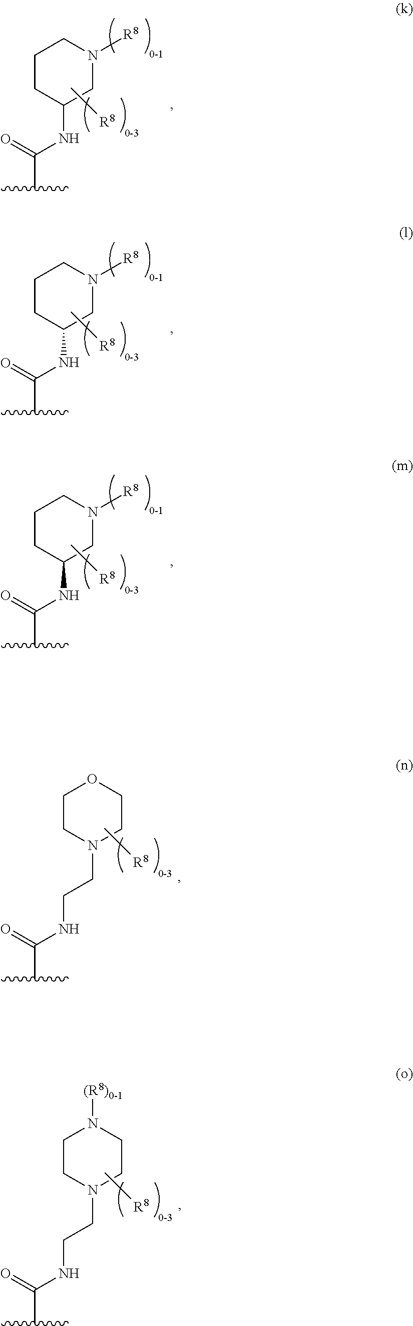

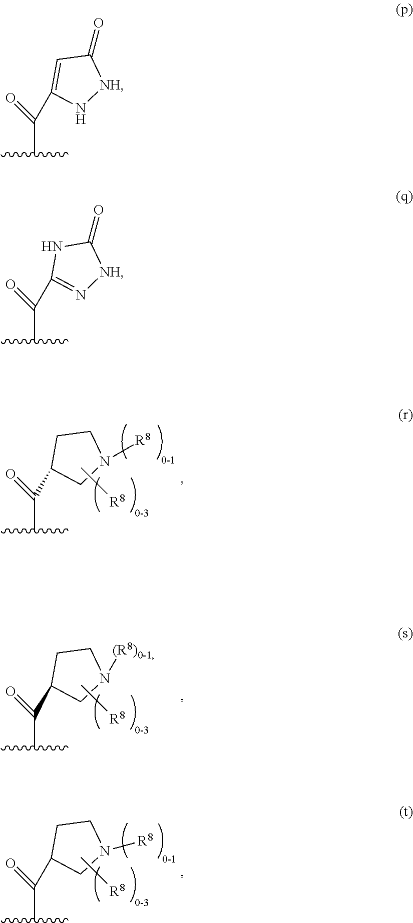

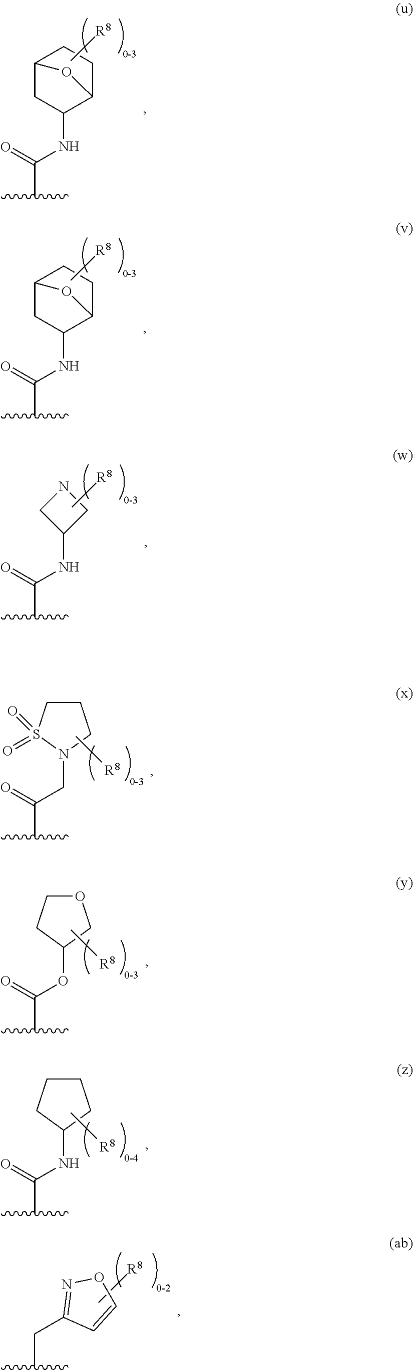

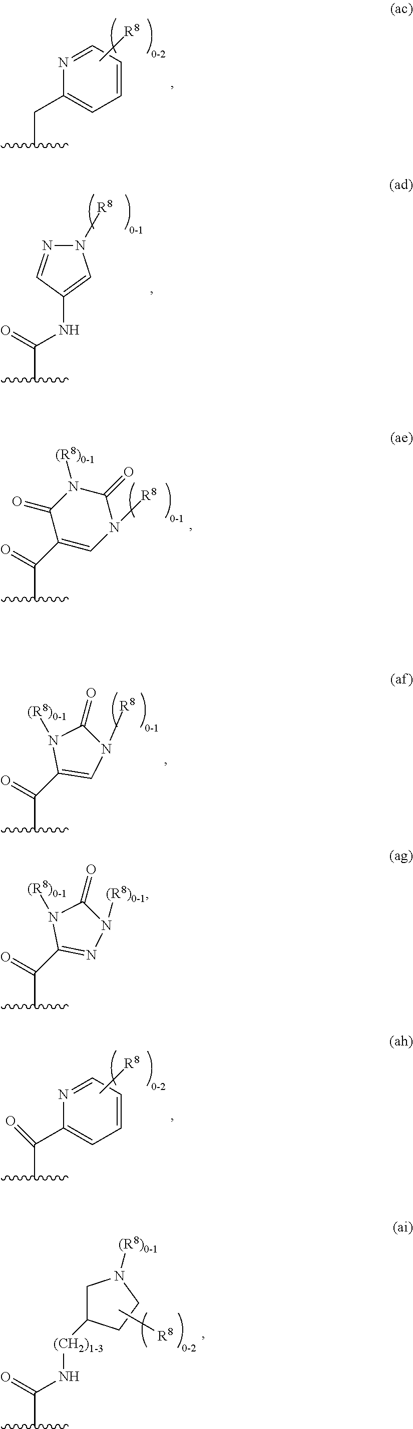

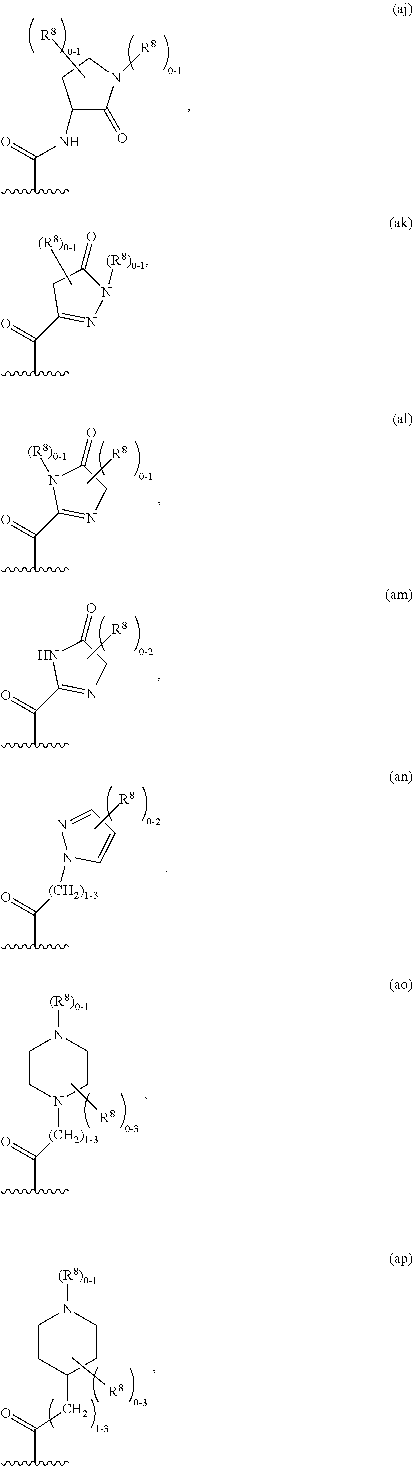

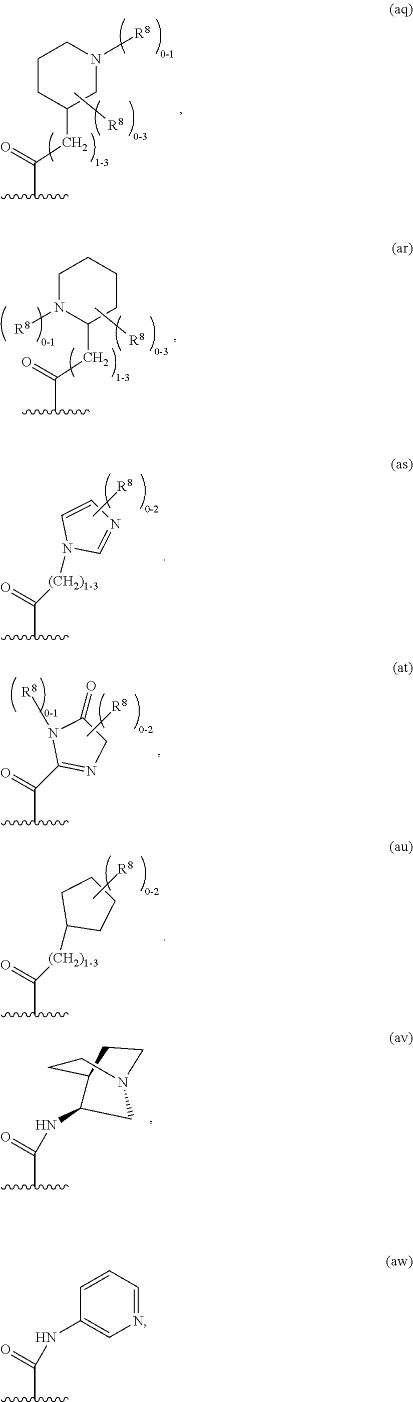

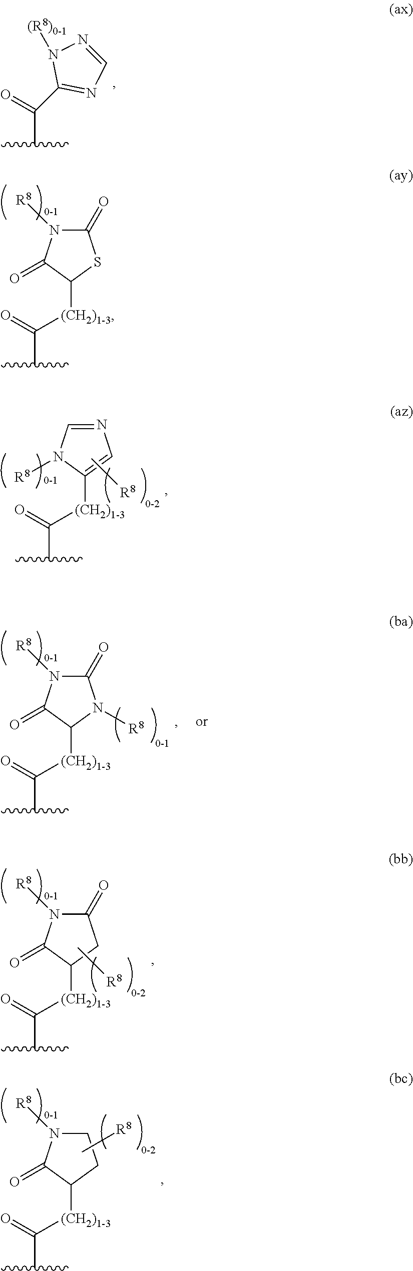

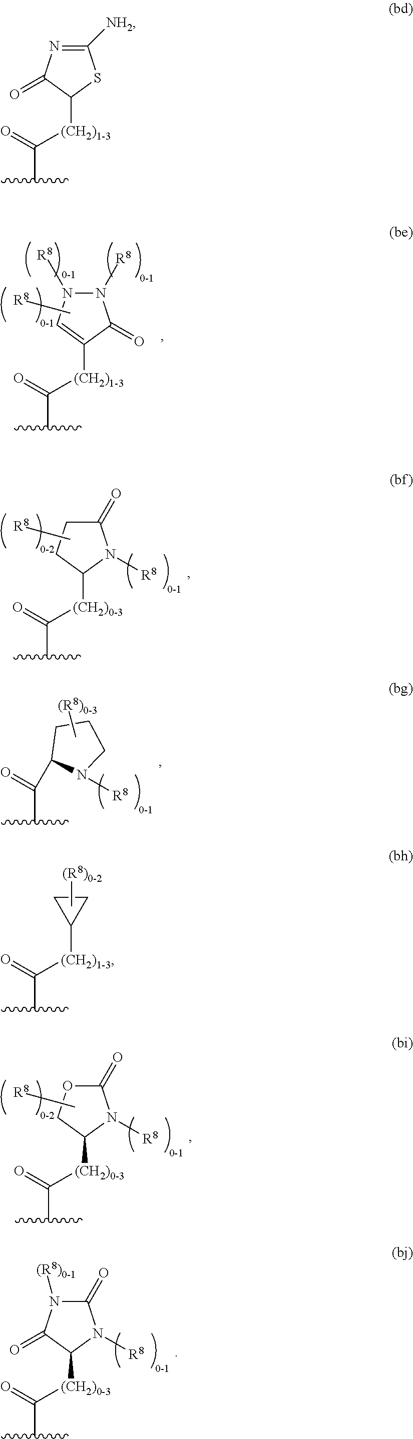

40. The compound according to claim 35, wherein G is cyanoalkylcarbonyl, -alkylene-N(R¹⁰)—C(O)O(R¹⁰) substituted with 0-1 R⁸, -alkylene-NH₂ with 0-1 R⁸, -alkylene-N(R¹⁰)C(O)-alkyl substituted with 1 R⁸, —C(O)—C₂-C₄alkenylene, —C₂-C₄alkenylene-C(O)N(R¹⁰)₂, -alkylene-N(R¹⁰)—SO₂(R¹⁰), -alkylene-N(R¹⁰)—C(O)—N(R¹⁰)₂, -alkylene-C(O)—N(R¹⁰)₂, -alkylene-C(O)OR¹⁰, —C(O)—NH₂, —SO₂N(R¹⁰)₂, —C(O)-alkylene-P(O)(R¹⁰)₂.

41. The compound according to claim 35, wherein R² is not cyanoalkyl, alkylcarbonylaminoalkyl, alkylaminocarbonyl, alkylaminoalkyl, substituted or unsubstituted C₃-C₆cycloalkyl, saturated 5-6 membered heterocycloalkyl with at least one heteroatom selected from the group consisting of N, S and O, or partially unsaturated 8-10 membered heterocycloalkyl with at least one heteroatom selected from the group consisting of N, S and O.

42. The compound according to claim 35, wherein R² is saturated 7-9 membered heterocycloalkyl comprising at least one nitrogen.

43. The compound according to claim 35, wherein R² is 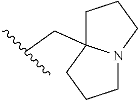 [SMILES: *CC12CCCN1CCC2]
that is substituted with 0-3 R⁴, provided that G is not alkyl, hydroxyalkyl, alkenyl, benzyl, alkoxyalkyl, alkylaminoalkyl, alkylcarbonyl, alkylcarbonylaminoalkyl, cycloalkyl, heterocycloalkyl, bridged heterocycloalkyl, heterocyclic spiro ring, aryl, or heteroaryl.

44. The compound according to claim 35, wherein when G is H, then R² is 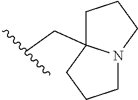 [SMILES: *CC12CCCN1CCC2]
that is substituted with 0-3 R⁴.

45. The compound according to claim 35 having Formula IIB or IID, or a pharmaceutically acceptable salt, a solvate, a tautomer, a stereoisomer, or a deuterated analog thereof.

46. The compound according to claim 33, having one of the following formulae: 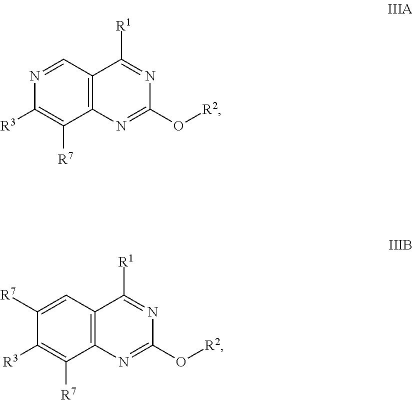
or a pharmaceutically acceptable salt, a solvate, a tautomer, a stereoisomer, or a deuterated analog thereof, wherein each R⁷ is independently H, F, Cl, C₁-C₃alkyl, or C₁-C₃haloalkyl.

47. The compound according to claim 33, having one of the following formulae: 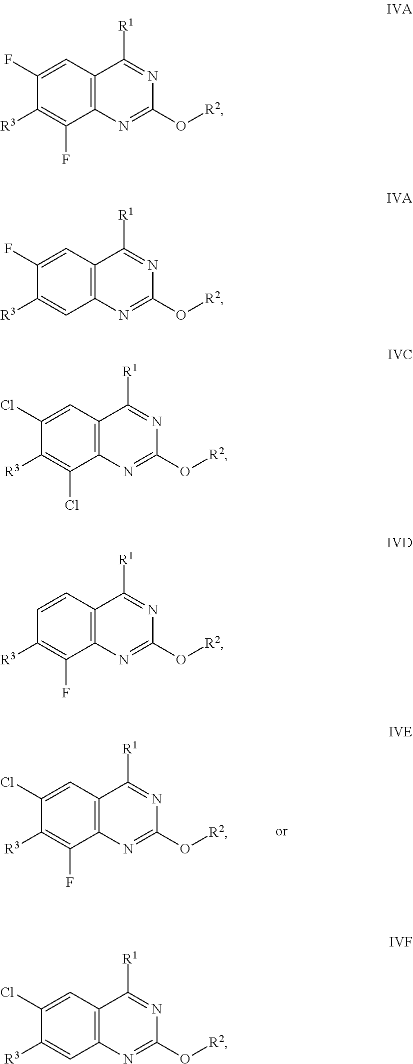
or a pharmaceutically acceptable salt, a solvate, a tautomer, a stereoisomer, or a deuterated analog thereof.

48. The compound according to claim 33, wherein:
R¹ is a 7-9 membered bridged heterocycloalkyl having at least one nitrogen atom, wherein R¹ is substituted with 0-3 T¹ and 1 G;
R² is —C₀-C₅alkyl-4-10 membered heterocycloalkyl substituted with 0-4 R⁴ and 0-1 J;
—C₀-C₅alkyl-5-10 membered heteroaryl substituted with 0-3 R⁴ and 0-1 J; or C₁-C₆alkyl substituted with 1-2 J groups;
R³ is phenyl, naphthyl, 5-10 membered heteroaryl, or 5-10 membered heterocycloalkyl, wherein the naphthyl, 5-10 membered heteroaryl, and 5-10 membered heterocycloalkyl are each substituted with 0-4 R⁵, and wherein the phenyl is substituted with 0-4 R⁵ and 0-1M;
each R⁴ is independently C₁-C₆alkyl, halogen, hydroxyl, CN, C₁-C₅haloalkyl, C₁-C₅hydroxyalkyl, C₁-C₅cyanoalkyl, or C₁-C₅alkoxy, provided that when R⁴ is attached to a nitrogen atom, R⁴ is not halogen, hydroxyl, or CN;
each R⁵ is independently C₁-C₅alkyl, C₁-C₅alkynyl, C₁-C₅alkoxy, halogen, hydroxyl, CN, C₁-C₅haloalkyl, C₁-C₅cynoalkyl, or C₁-C₅hydroxyalkyl;
each R⁸ is independently C₁-C₅alkyl, halogen, hydroxyl, —NH₂, —N(H)C₁-C₅alkyl, —N(C₁-C₅alkyl)₂, CN, C₁-C₅haloalkyl, or C₁-C₅hydroxyalkyl, provided that when R⁸ is attached to nitrogen, R⁸ is not halogen, hydroxyl, —NH₂, —N(H)C₁-C₅ alkyl, —N(C₁-C₅alkyl)₂, or CN;
each R⁹ is independently halogen, hydroxyl, CN, C₁-C₅haloalkyl, or C₁-C₅hydroxyalkyl;
each R¹⁰ is independently H, C₁-C₆alkyl, or C₁-C₅haloalkyl;
each T¹ is independently C₁-C₆alkyl, halogen, hydroxyl, C₁-C₅haloalkyl, or C₁-C₅hydroxyalkyl, provided that when T¹ is attached to nitrogen, T¹ is not halogen or hydroxyl;
T² is H, CN, —C(O)OR¹⁰, C₁-C₅alkoxy, N(R¹⁰)₂, —C(O)N(R¹⁰)₂, —SO₂N(R¹⁰)₂, —SO₂R¹⁰, —N(R¹⁰)C(O)—C₁-C₅alkyl, —O—C(O)—C₁-C₅alkyl, C₁-C₅cycloalkyl, 4-10 membered heterocycloalkyl, 7-9 membered bridged heterocycloalkyl, phenyl, naphthyl, or 5-10 membered heteroaryl, wherein T² substituted with 0-4 R⁸,
provided that when L¹ and L² are both absent, T² is not CN, C₁-C₅alkoxy, N(R¹⁰)₂, —N(R¹⁰)C(O)—C₁-C₅alkyl, or —O—C(O)—C₁-C₅alkyl, and provided that when L¹ and L² taken together are —C(O)—, —C(O)N(H)—, —C₁-C₅alkyelene-C(O)N(H)—, C₁-C₅alkylene-C(O)—, —SO₂—, or —SO₂NH, T² is not CN, —C(O)OR¹⁰, C₁-C₅alkoxy, N(R¹⁰)₂, —C(O)N(R¹⁰)₂, —SO₂N(R¹⁰)₂, —N(R¹⁰)C(O)—C₁-C₅alkyl, or —O—C(O)—C₁-C₅alkyl;
L¹ is absent, —C(O)—, —C(O)N(H)—, —C₁-C₅alkyelene-C(O)N(H)—, —C₁-C₅alkylene-C(O)—, —SO₂—, or —SO₂N(H)—; and
L² is absent, or C₁-C₆alkylene substituted with 0-4 R⁹, provided that when L¹ is —C(O)— and L² is absent, T² is not H; and
M is —C(O)NH₂, —NH₂C(O)CH₃, —N(H)SO₂CH₃, or —NH₂, provided that M is not attached to a nitrogen atom.

49. The compound according to claim 33, wherein:
R¹ is a 7-9 membered bridged heterocycloalkyl having at least one nitrogen atom, wherein R¹ is substituted with 0-2 T¹ and 1 G;
R² is 5-10 membered heterocyloalkyl-C₁-C₄alkyl substituted with 0-3 R⁴ or C₁-C₅alkyl substituted with 1-2 J groups;
R³ is phenyl, naphthyl, 5-10 membered heteroaryl, or a 5-10 membered heterocycloalkyl, wherein the naphthyl, 5-10 membered heteroaryl, and 5-10 membered heterocycloalkyl are each substituted with 0-3 R⁵, and wherein the phenyl is substituted with 0-4 R⁵ and 0-1M;
each R⁴ is independently C₁-C₄alkyl, halogen, hydroxyl, CN, C₁-C₄haloalkyl, C₁-C₄hydroxyalkyl, or C₁-C₄alkoxy, provided that when R⁴ is attached to a nitrogen atom, R⁴ is not halogen, hydroxyl, or CN;
each R⁵ is independently C₁-C₄alkyl, C₁-C₄alkynyl, halogen, hydroxyl, CN, C₁-C₄haloalkyl, C₁-C₄cynoalkyl, or C₁-C₄hydroxyalkyl;
each R⁸ is independently C₁-C₄alkyl, halogen, hydroxyl, —NH₂, —N(H)C₁-C₄alkyl, —N(C₁-C₄alkyl)₂, CN, C₁-C₄haloalkyl, or C₁-C₄hydroxyalkyl, provided that when R⁸ is attached to nitrogen, R⁸ is not halogen, hydroxyl, —NH₂, —N(H)C₁-C₅alkyl, —N(C₁-C₅alkyl)₂, or CN;
each R⁹ is independently halogen, hydroxyl, CN, C₁-C₄haloalkyl, or C₁-C₄hydroxyalkyl;
each R¹⁰ is independently H, C₁-C₄alkyl, or C₁-C₄haloalkyl;
each T¹ is independently C₁-C₄alkyl halogen hydroxyl, C₁-C₄haloalkyl, or C₁-C₄hydroxyalkyl, provided that when T¹ is attached to nitrogen, T¹ is not halogen or hydroxyl;
T² is H, CN, —C(O)OR¹⁰, C₁-C₄alkoxy, —N(R¹⁰)₂, —C(O)N(R¹⁰)₂, SO₂N(R¹⁰)₂, —SO₂R¹⁰, —N(R¹⁰)C(O)—C₁-C₄alkyl, —O—C(O)—C₁-C₄alkyl, C₃-C₆cycloalkyl, 4-6 membered heterocycloalkyl, 7-8 membered bridged heterocycloalkyl, phenyl, naphthyl, or 5-6 membered heteroaryl, wherein T² is substituted with 0-4 R⁸,
provided that when L¹ and L² are both absent, T² is not CN, C₁-C₄alkoxy, N(R¹⁰)₂, —N(R¹⁰)C(O)—C₁-C₄alkyl, or —O—C(O)—C₁-C₄alkyl, and provided that when L¹ and L² taken together are —C(O)—, —C(O)N(H)—, —S(O)₂—, C₁-C₄alkyelene-C(O)N(H)—, or —C₁-C₄alkylene-C(O)—, T² is not CN, —C(O)OR¹⁰, C₁-C₄alkoxy, —N(R¹⁰)₂, —C(O)N(R¹⁰)₂, SO₂N(R¹⁰)₂, —N(R¹⁰)C(O)—C₁-C₄alkyl, or —O—C(O)—C₁-C₄alkyl;
L¹ is absent, —C(O)—, —C(O)N(H)—, —S(O)₂—, —SO₂N(H)—, C₁-C₄alkyelene-C(O)N(H)—, —C₁-C₄alkylene-C(O)—, or —C₁-C₄alkylene-C(O)—; and
L² is absent, or C₁-C₄alkylene substituted with 0-2 R⁹, provided that when L¹ is —C(O)— and L² is absent, T² is not H.

50. The compound according to claim 49, wherein G is H.

51. The compound according to claim 49, wherein G is not H.

52. The compound according to claim 49, wherein G is not hydrogen, alkyl, hydroxyalkyl, alkenyl, benzyl, alkoxyalkyl, alkylaminoalkyl, alkylcarbonyl, alkylcarbonylaminoalkyl, cycloalkyl, heterocycloalkyl, bridged heterocycloalkyl, heterocyclic spiro ring, aryl, or heteroaryl.

53. The compound according to claim 33, wherein R¹ is 7-9 membered bridged piperazine or piperidine substituted with 0-2 T¹ and 1 G.

54. The compound according to claim 33, wherein R¹ is one of the following formulae:

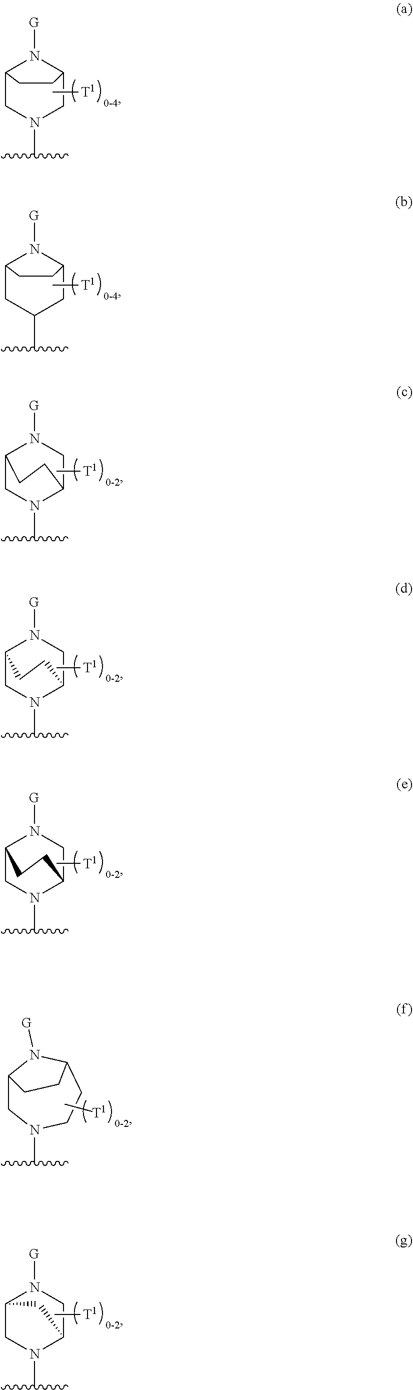

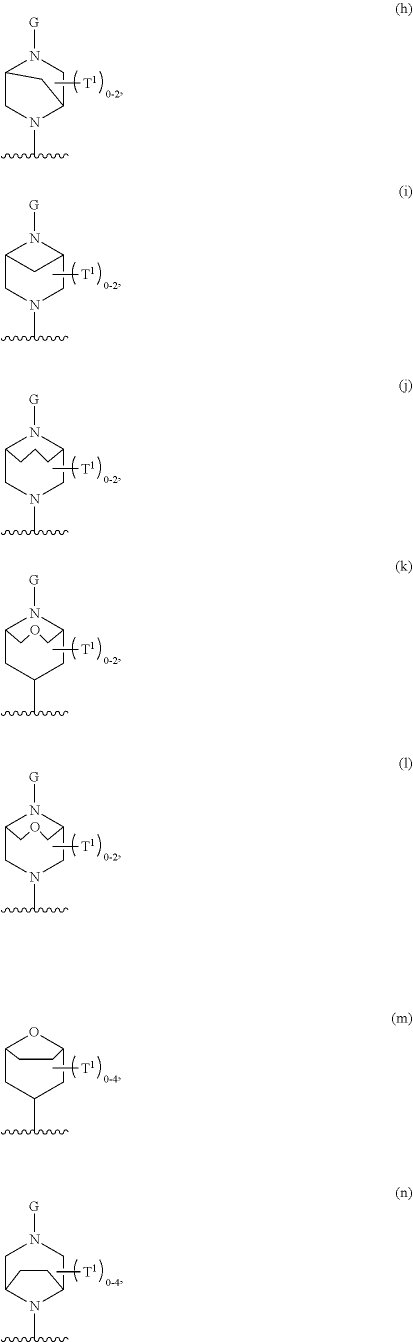

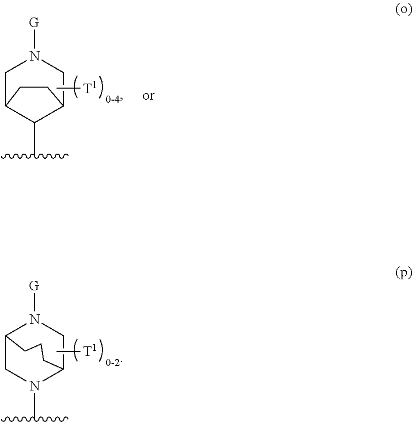

55. The compound according to claim 54, wherein R¹ is formula (a) or (b).

56. The compound according to claim 33, wherein R² is one of the following formulae:

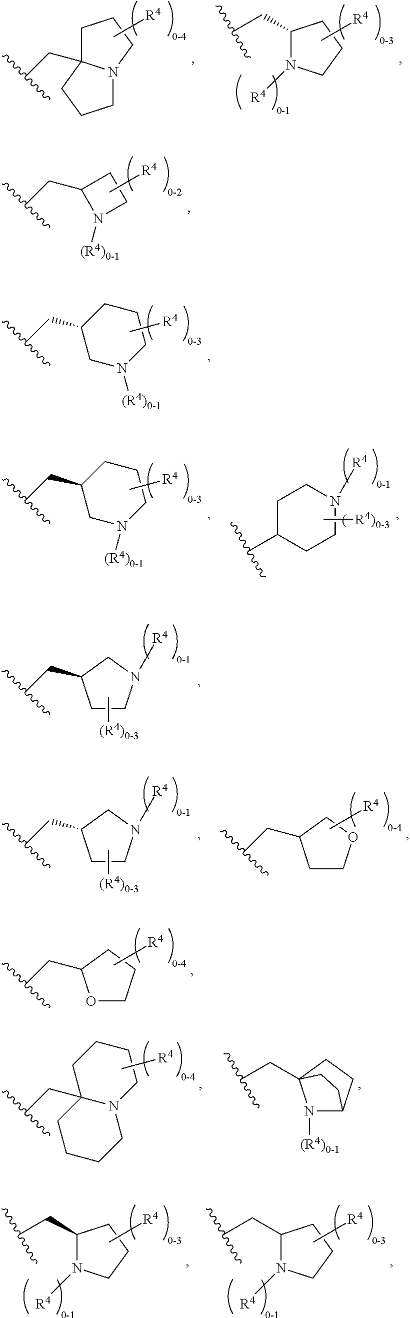

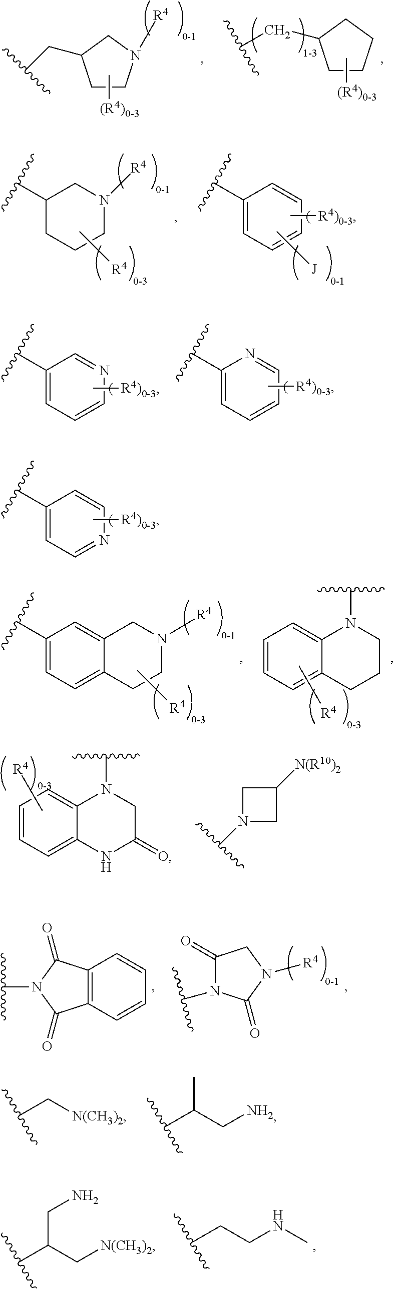

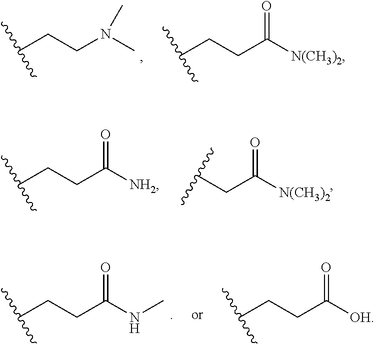

57. The compound according to claim 33, wherein R² is one of the following formulae and R⁴ is H, Cl, or F:

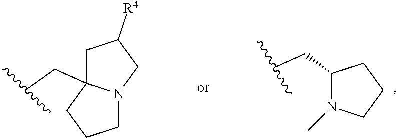

58. The compound according to claim 33, wherein R³ is one of the following formulae:

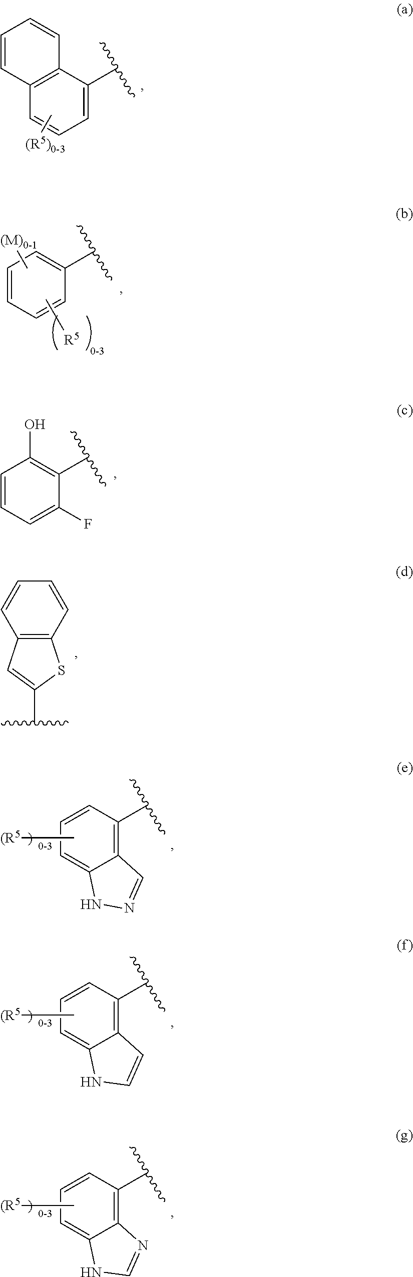

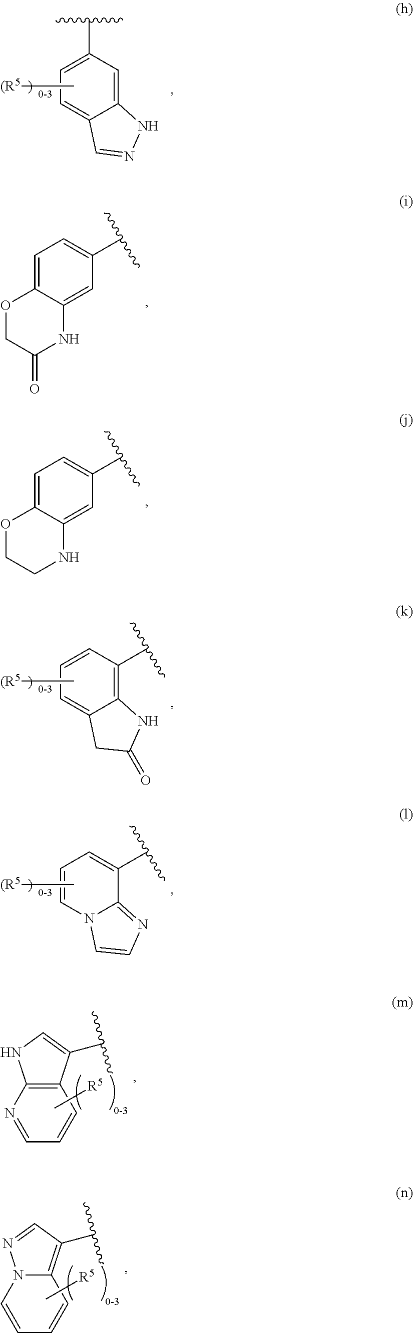

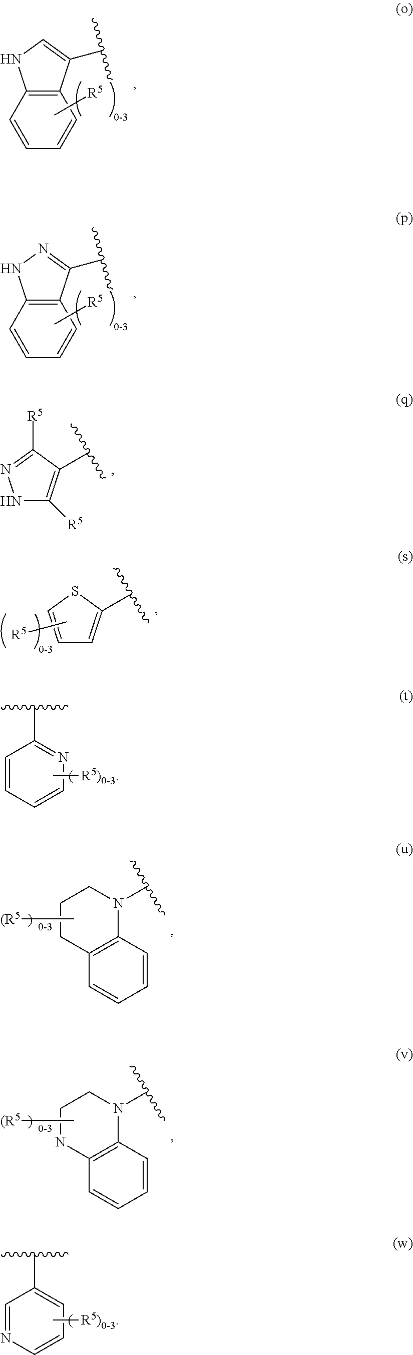

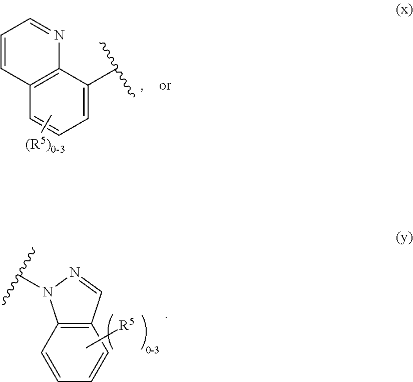

59. The compound according to claim 33, wherein R³ is 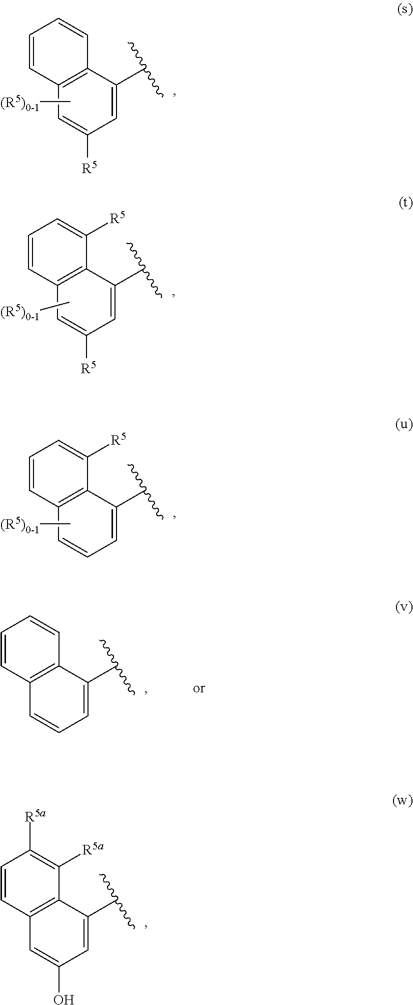
and each R^{5a} is independently hydrogen, halo, or ethynyl.

60. The compound according to claim 33, wherein R³ is: 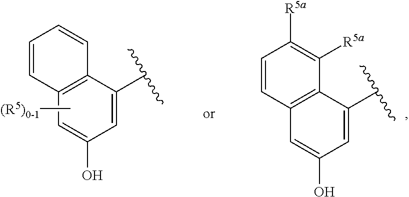
and each R^{5a} is independently hydrogen, halo, or ethynyl.

61. The compound according to claim 33, wherein:
L¹ is —C(O)N(H)—, —C(O)—, —C₁-C₃alkylene, —SO₂—, —SO₂N(H)—, or —C₁-C₃alkylene-C(O)—;
L² is absent or C₁-C₃alkylene; and
T² is one of the following formulae:

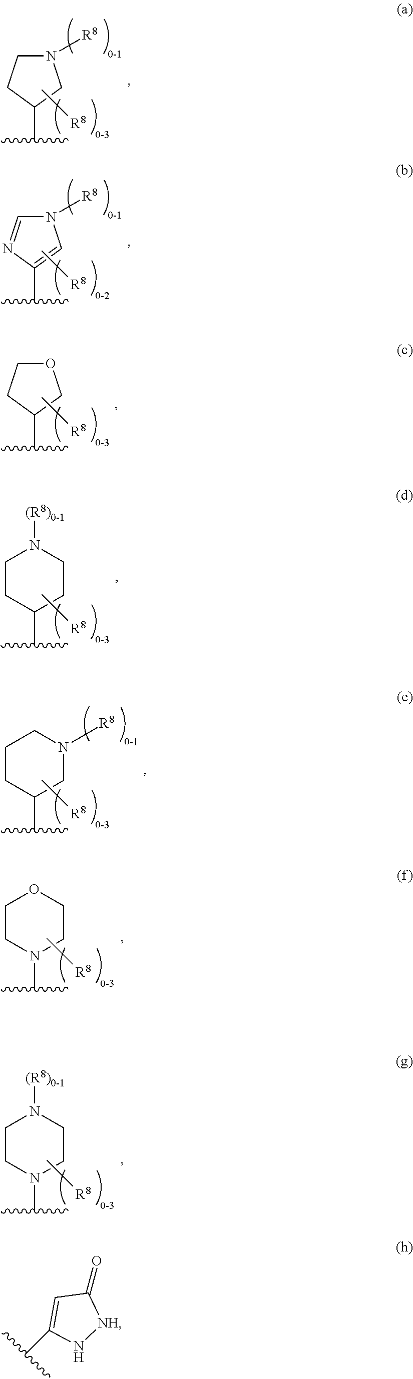

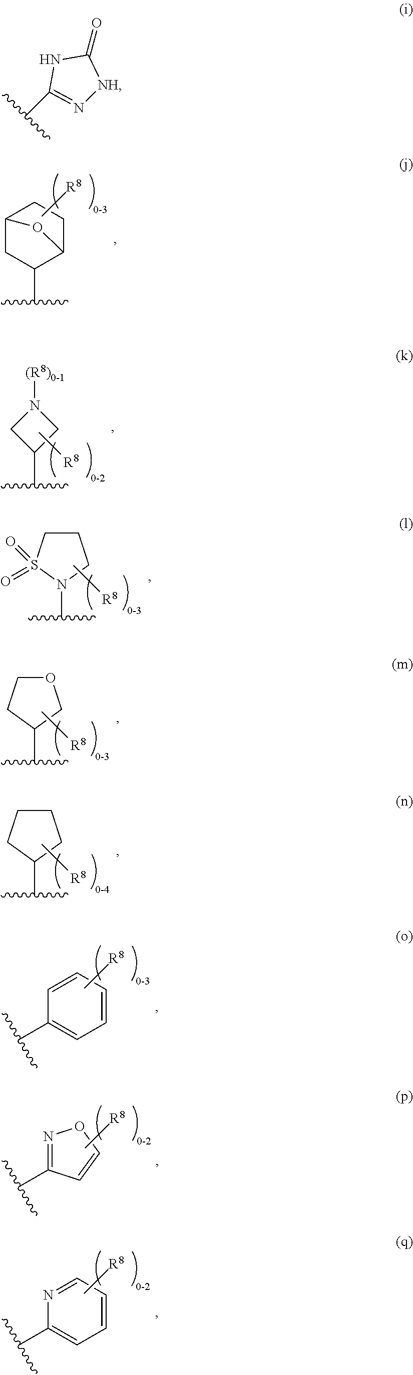

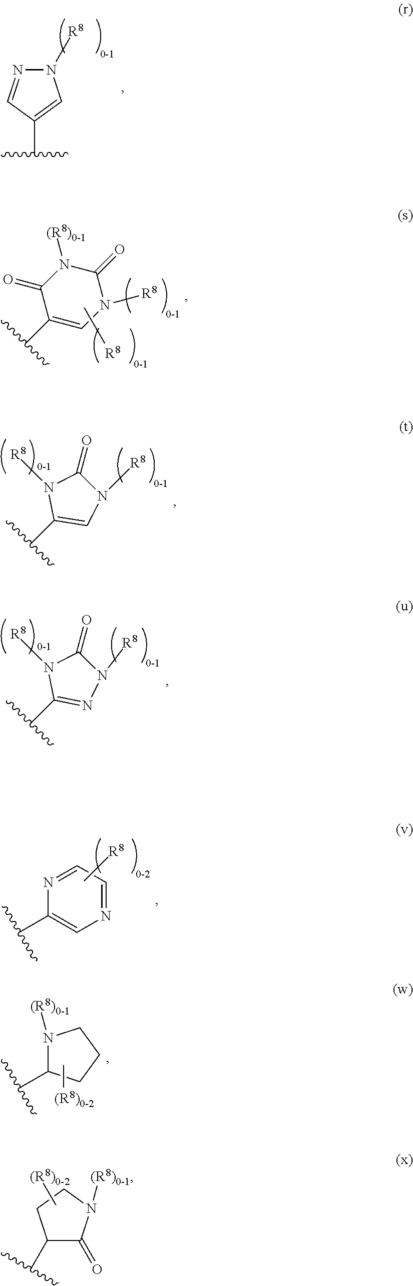

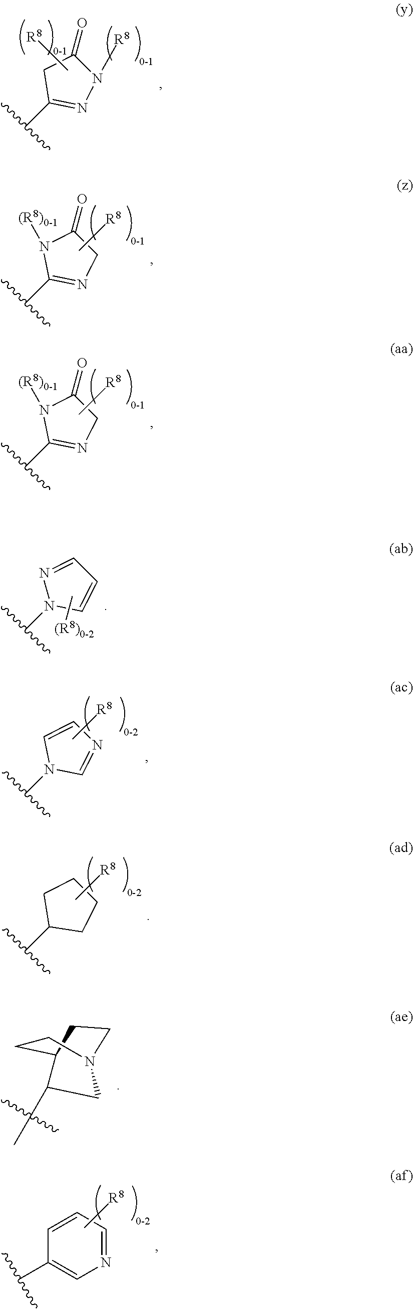

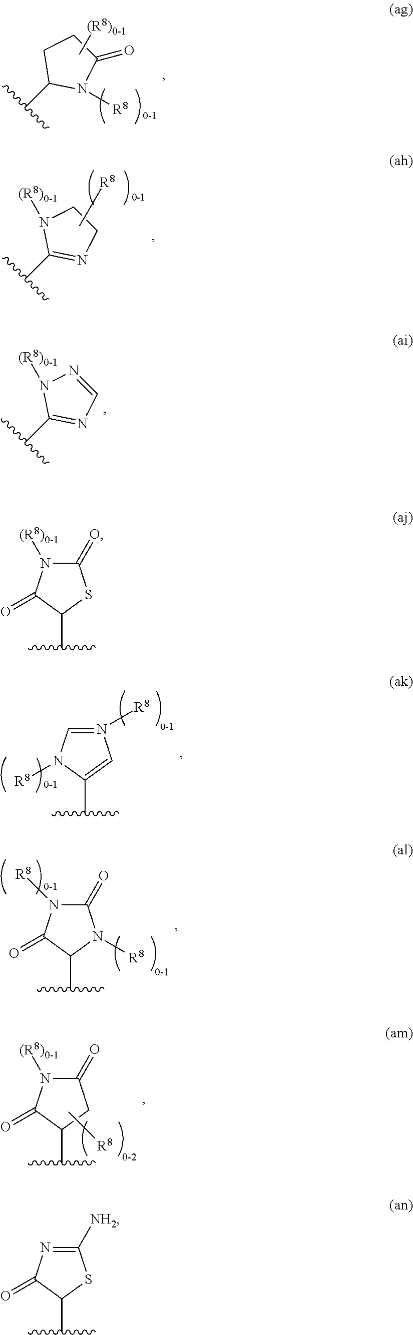

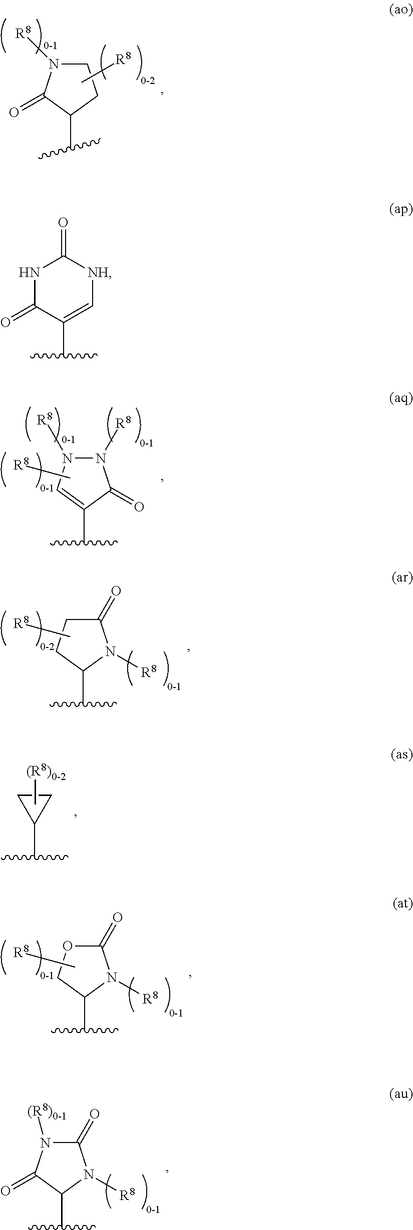

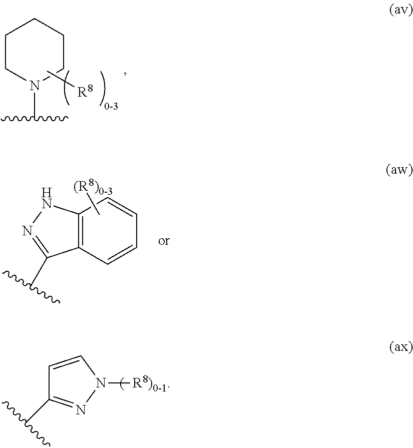

62. The compound according to claim 33, wherein:
L¹ is —C(O)N(H)— or —C(O)—; and
L² is absent, —CH₂—, or —CH₂—CH₂—.

63. The compound according to claim 33, wherein:
L¹ is absent;
L² is absent; and
T² is H.

64. The compound according to claim 33, wherein G is:
—CH₂—C(H)(OH)—CH₂—OH, —C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, —C(O)(CH₂)₂C(O)OCH₃, or one of the following Formulae:

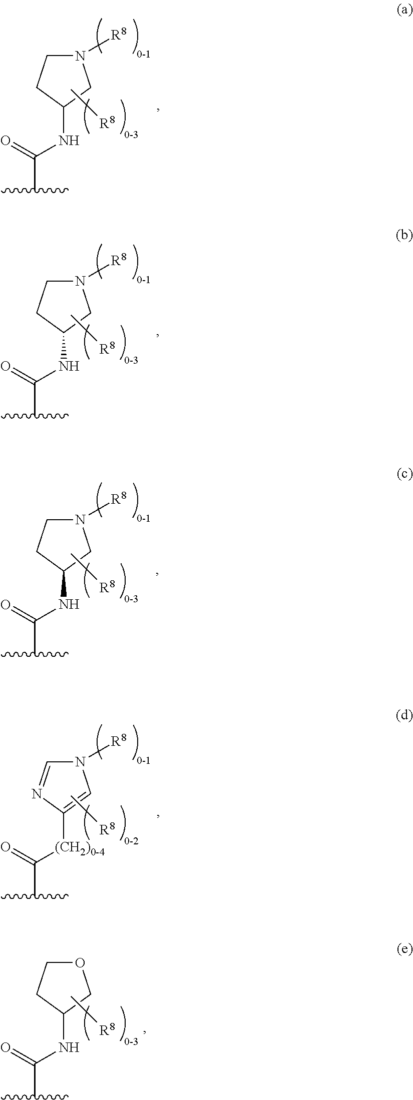

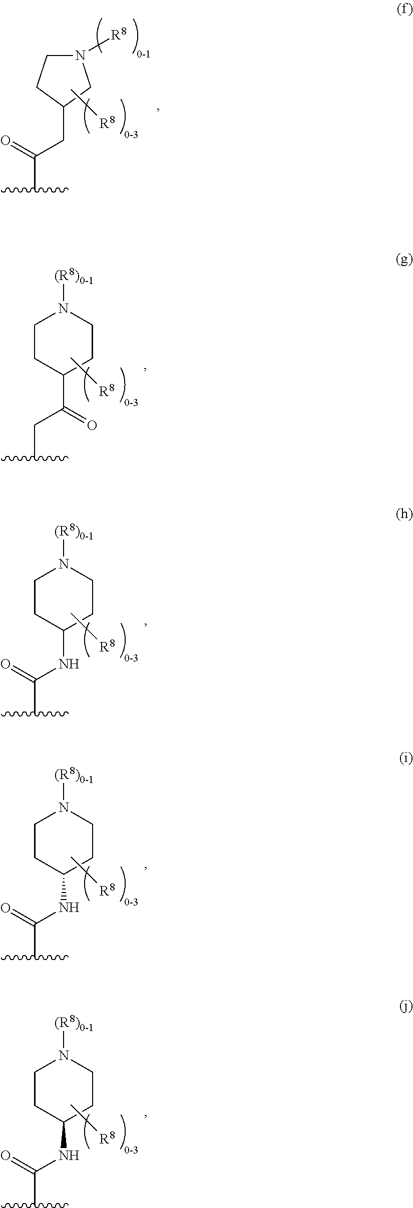

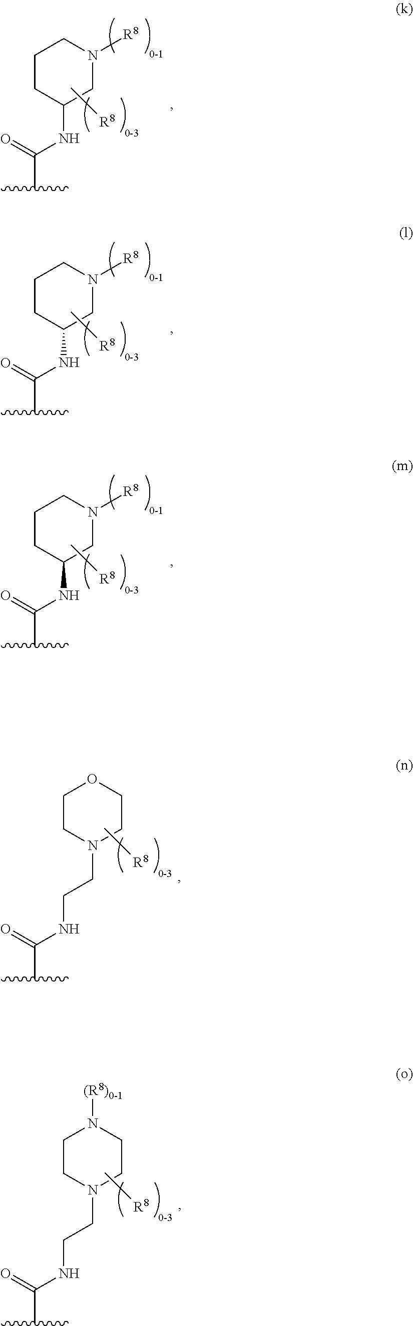

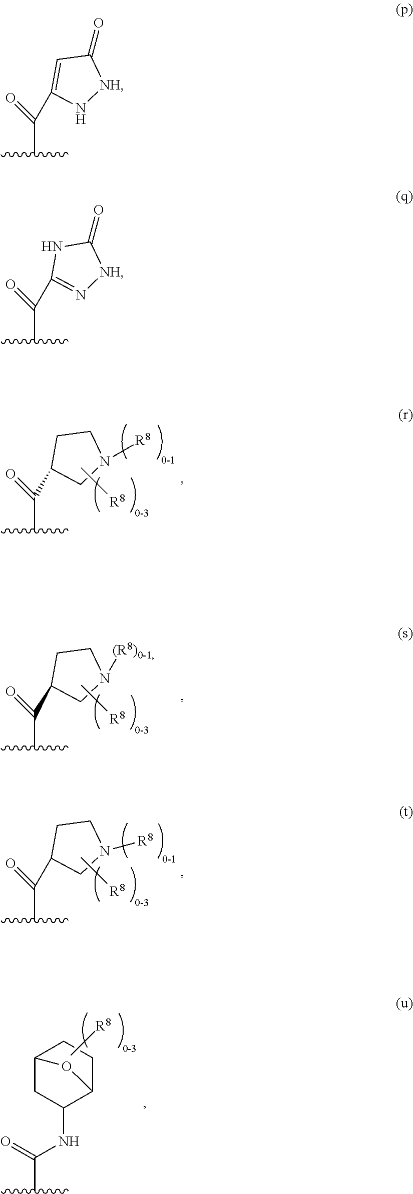

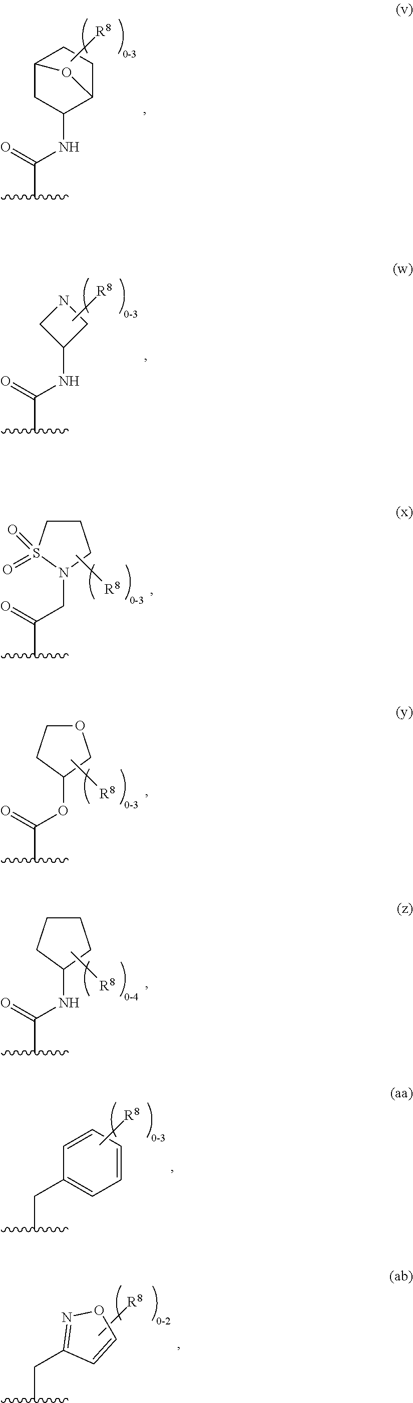

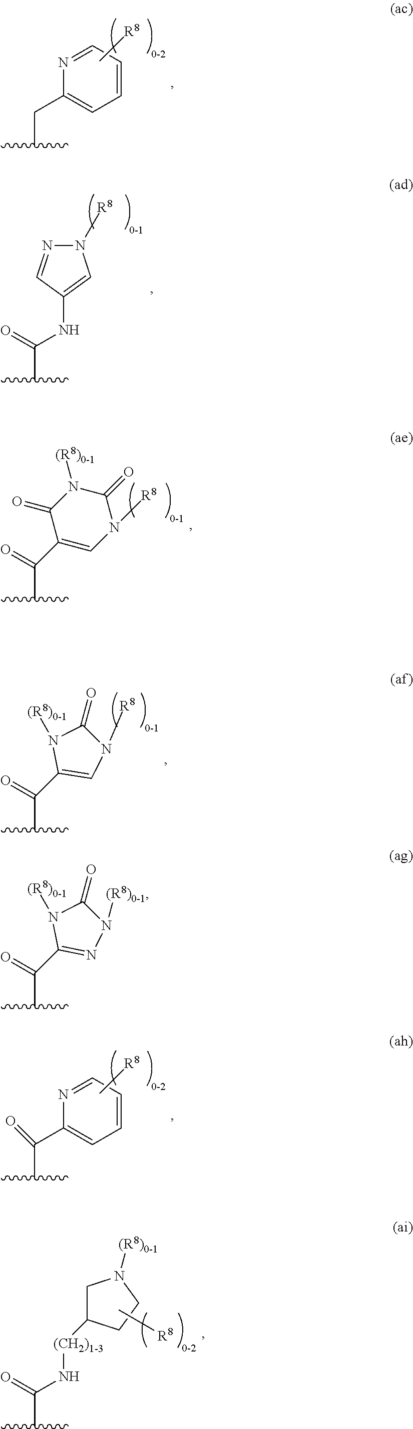

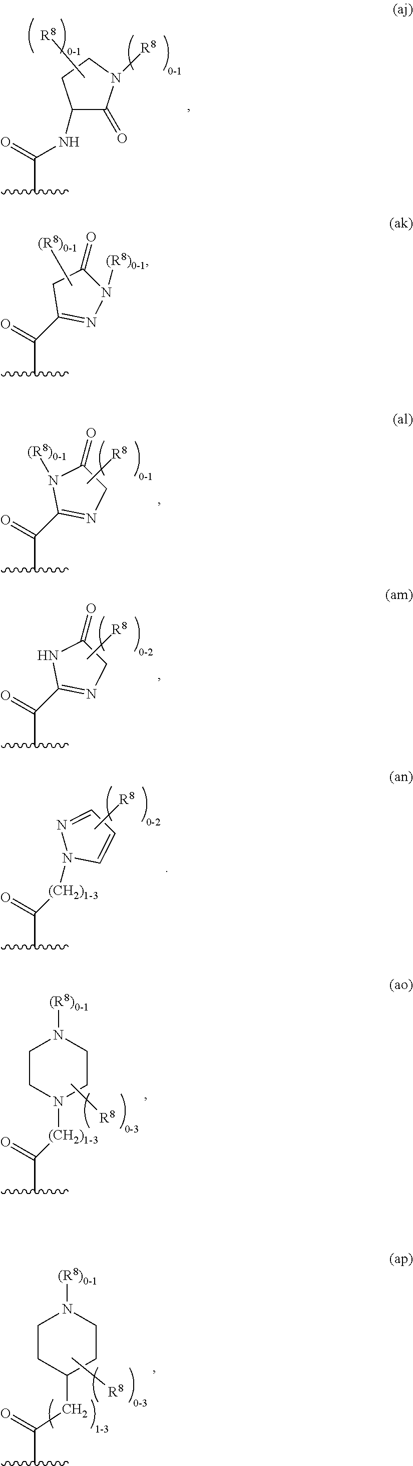

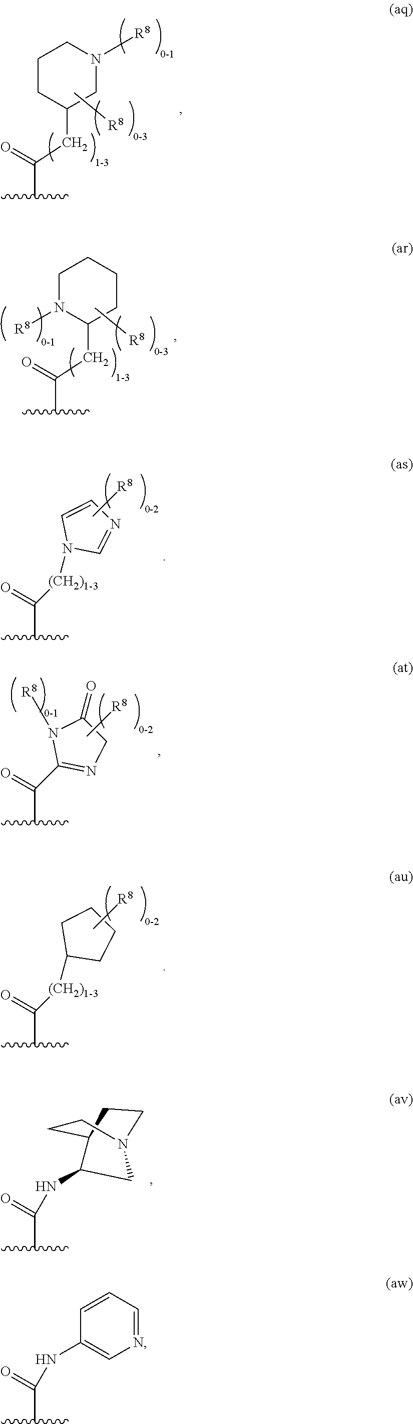

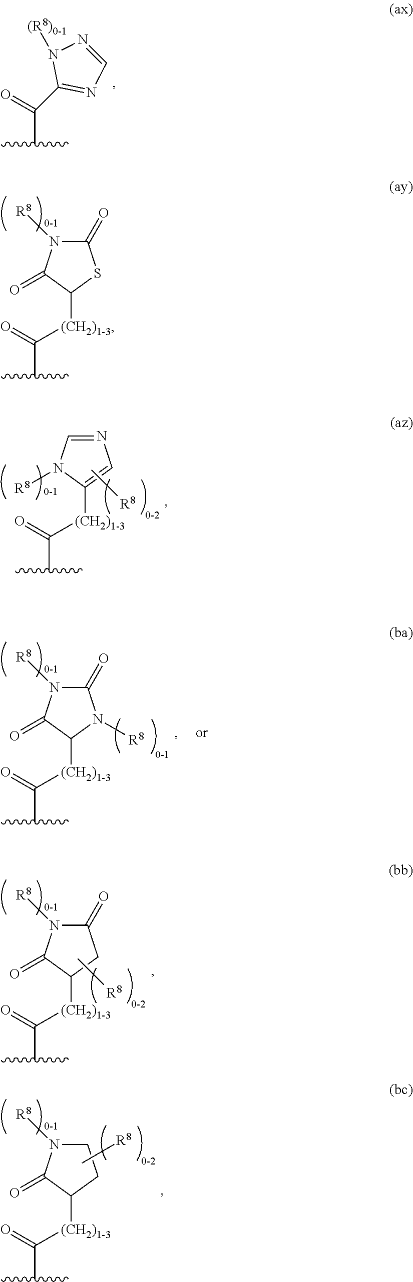

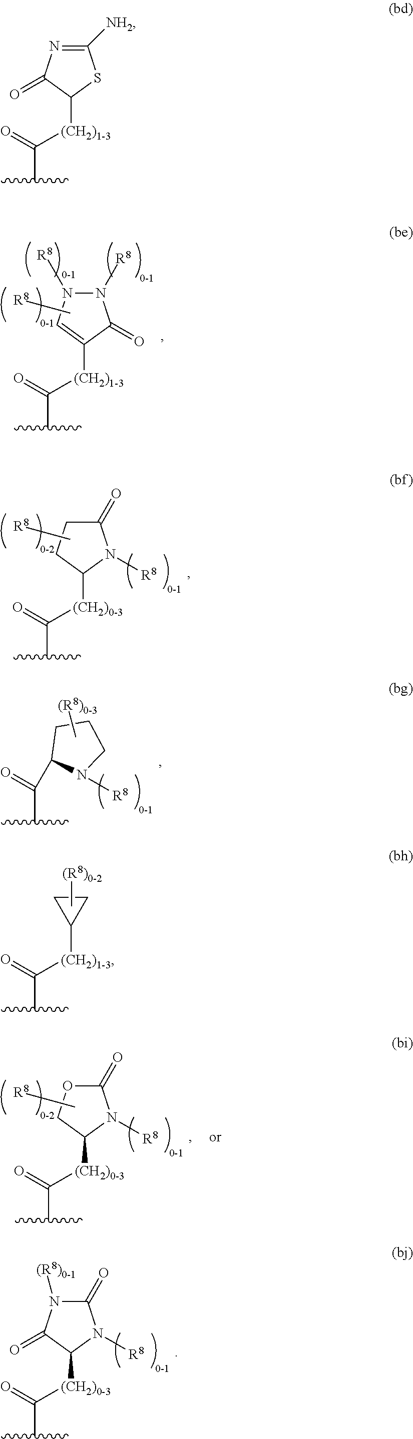

65. The compound according to claim 64, wherein G is —CH₂—C(H)(OH)—CH₂—OH, —C(O)N(H)CH₂COOH, —S(O)₂(CH₂)₂OCH₃, —C(O)CH₂—SO₂CH₃, —C(O)CH₂—SO₂—NH₂, —C(O)C(H)(CH₃)—SO₂—NH₂, —C(O)(CH₂)₂—SO₂—NH₂, —C(O)CH₂—SO₂—N(CH₃)₂, —SO₂N(H)(CH₂)₂CH₃, —SO₂N(H)(CH₂)₂N(CH₃)₂, —SO₂(CH₂)₃N(CH₃)₂, —C(O)CH₂CH(OH)CH₂NH₂, —C(O)CH₂C(OH)(CH₃)₃, —C(O)CH₂CH₂OH, —C(O)CH(CH₃)OH, —C(O)CH(OH)(CH₂)OH, —C(O)CH₂N(H)C(O)CH₃, —C(O)NH₂, —C(O)N(H)CH₂C(O)OCH₂CH₃, —C(O)CH₂NH₂, —C(O)CH₂CN, —C(O)(CH₂)₂CN, —C(O)CH₂NHCH₃, —C(O)CH₂CH₃, —C(O)CH₂N(CH₃)₂, —C(O)CH₂CH₂CH₂N(CH₃)₂, —C(O)N(H)CH₂C(CH₃)₂OH, —C(O)N(H)CH₂C(O)NH₂, —C(O)CH₂C(O)OH, —C(O)(CH₂)₂C(O)OH, —C(O)CH₂OC(O)CH₃, —C(O)CH₂OH, or —C(O)(CH₂)₂C(O)OCH₃.

66. The compound according to claim 64, wherein G is one of formulae (a), (b), (c), (d), (e), (h), (i), (j), (k), (l), (m), (p), (q), (r), (s), (t), (u), (v), (w), (z), (ad), (ae), (af), (ag), (ah), (aj), (ak), (al), (am), (at), (av), (aw), (ax), or (bg).

67. The compound according to claim 64, wherein G is one of formulae (d), (f), (n), (o), (x), (ai), (an), (ao), (ap), (aq), (ar), (as), (au), (ay), (az), (ba), (bb), (be), (bd), (be), (bf), (bi), or (bj).

68. The compound according to claim 64, wherein R² is 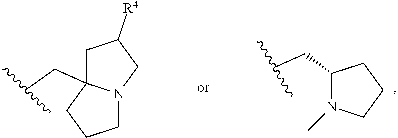
and R⁴ is H, Cl, or F.

69. A compound having Formula F 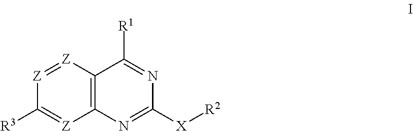
or a pharmaceutically acceptable salt, a solvate, a tautomer, a stereoisomer, or a deuterated analog thereof, wherein:
R¹ is bridged heterocycloalkyl substituted with 0-4 T¹ and 1 G;
R² is alkyl substituted with 0-5 R⁴ and 0-2 J, aryl substituted with 0-4 R⁴ and 0-1 J, arylalkyl substituted with 0-4 R⁴ and 0-1 J, heteroaryl substituted with 0-5 R⁴, heteroarylalkyl substituted with 0-5 R⁴, heterocycloalkyl substituted with 0-5 R⁴, heterocycloalkylalkyl substituted with 0-5 R⁴ and 0-1 J, bridged heterocycloalkyl substituted with 0-5 R⁴, bridged heterocycloalkylalkyl substituted with 0-5 R⁴ and 0-1 J, cycloalkyl substituted with 0-5 R⁴ and 0-1 J; or cycloalkylalkyl substituted with 0-5 R⁴ and 0-1 J; provided that when R² is alkyl substituted with 0-5 R⁴ and 0-2 J, X is not —C[(R¹⁰)₂]₁₋₆;
R³ is aryl, heteroaryl or heterocycloalkyl, wherein R³ is substituted with 0-4 R⁵ and 0-1 M;
each R⁴ is independently alkyl, halogen, hydroxyl, CN, haloalkyl, hydroxyalkyl, or alkoxy, provided that when R⁴ is attached to a nitrogen atom, R⁴ is not halogen, hydroxyl or CN;
each R⁵ is independently alkyl, halogen, hydroxyl, CN, haloalkyl or hydroxyalkyl, provided that when R⁵ is attached to a nitrogen atom, R⁵ is not halogen, hydroxyl or CN;
each R⁶ is independently H, alkyl, haloalkyl or hydroxyalkyl;
each R⁷ is independently H, halogen, alkyl, alkenyl, alkynyl, CN, haloalkyl or hydroxyalkyl;
cycloalkyl, —C(O)OR¹⁰, alkoxy optionally substituted with 1-3 halogens;
each R⁸ is independently alkyl, halogen, hydroxyl, —NH₂, CN, haloalkyl or hydroxyalkyl, provided that when R⁸ is attached to nitrogen, R⁸ is not halogen, hydroxyl, —NH₂ or CN;
each R⁹ is independently halogen, hydroxyl, CN, haloalkyl or hydroxyalkyl;
each R¹⁰ is independently H, alkyl, hydroxyalkyl or haloalkyl;
each J is independently is —N(R¹⁰)₂, —C(O)N(R¹⁰)₂, or —C(O)OR¹⁰;
M is —C(O)NH₂, —N(H)C(O)CH₃, —N(H)SO₂-alkyl, or —N(R¹⁰)₂;
X is absent, —C[(R¹⁰)₂]₁₋₆—, —C[(R¹⁰)₂]₀₋₃—O— or —NR⁶—;
each Z is independently CR⁷ or N;
G is L¹-L²-T²;
each T¹ is independently alkyl halogen hydroxyl, haloalkyl or hydroxyalkyl, provided that when T¹ is attached to nitrogen, T¹ is not halogen or hydroxyl;
T² is H, —C(O)OR¹⁰, —N(R¹⁰)₂, —OR¹⁰, —C(O)N(R¹⁰)₂, —SO₂N(R¹⁰)₂, —N(R¹⁰)C(O)-alkyl, —O—C(O)-alkyl, —C(═NR¹⁰)N(R¹⁰)₂, cycloalkyl, heterocycloalkyl, bridged heterocycloalkyl, heterocyclic spiro ring, carbocyclic spiro ring, aryl or heteroaryl, wherein T² is substituted with 0-4 R⁸, provided that when L¹ and L² are both absent, T² is not N(R¹⁰)₂, —OR¹⁰, —N(R¹⁰)C(O)-alkyl or —O—C(O)-alkyl;
L¹ is absent, —C(O), —C(O)C(O)N(H)—, —C(O)O—, —C(O)N(H)—, —S(O)₂—, -alkylene-C(O)N(H)—, -alkylene-C(O)—, —SO₂—, or —SO₂NH—; and
L² is absent, - or alkylene substituted with 0-4 R⁹, provided that when L¹ is —C(O)— and L² is absent, T² is not H.

70. The compound according to claim 69 having one of the following formulae: 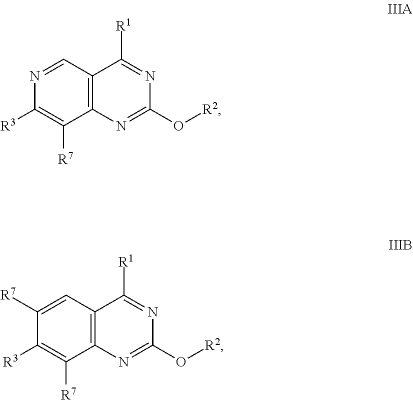
or a pharmaceutically acceptable salt, a solvate, a tautomer, a stereoisomer, or a deuterated analog thereof, wherein each R⁷ is independently H, F, Cl, C₁-C₃alkyl or C₁-C₃haloalkyl.

71. The compound according to claim 69 having one of the following formulae: 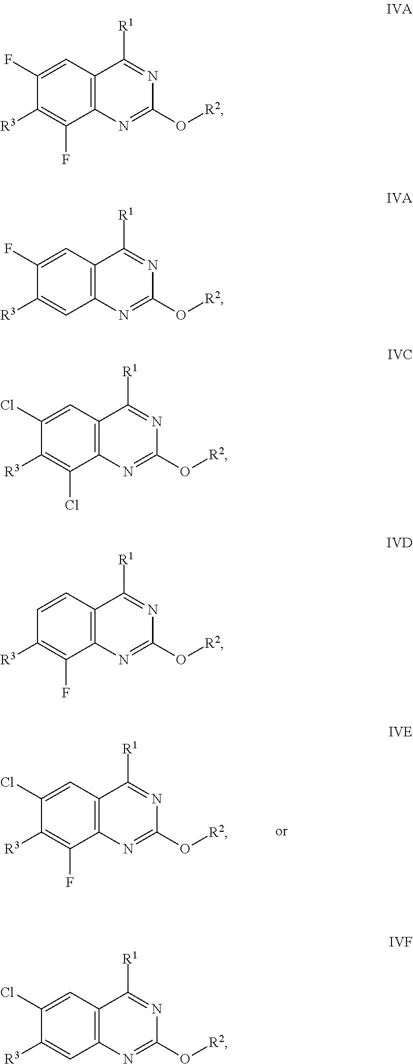
or a pharmaceutically acceptable salt, a solvate, a tautomer, a stereoisomer, or a deuterated analog thereof.

72. The compound according to claim 69, wherein R¹ is one of the following formulae:

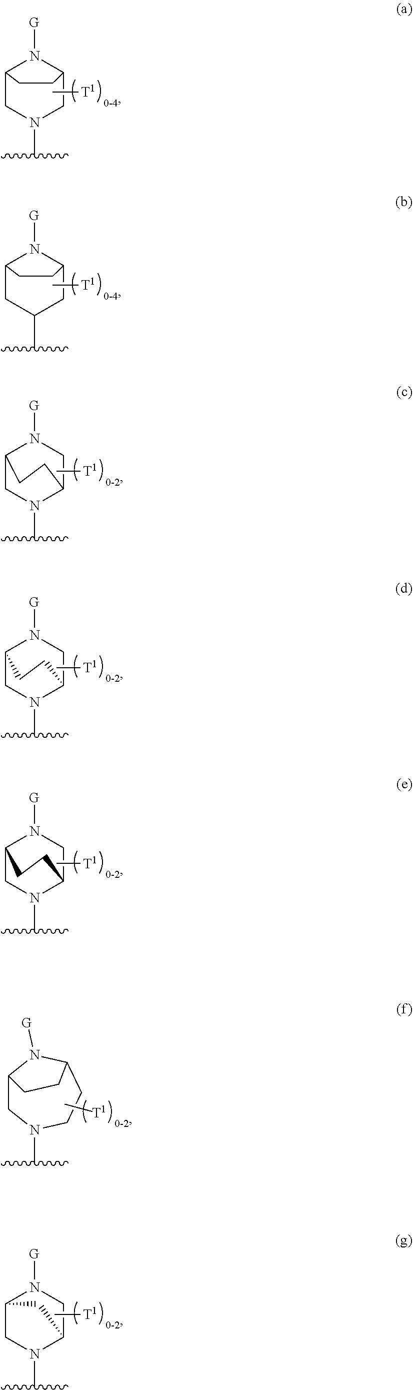

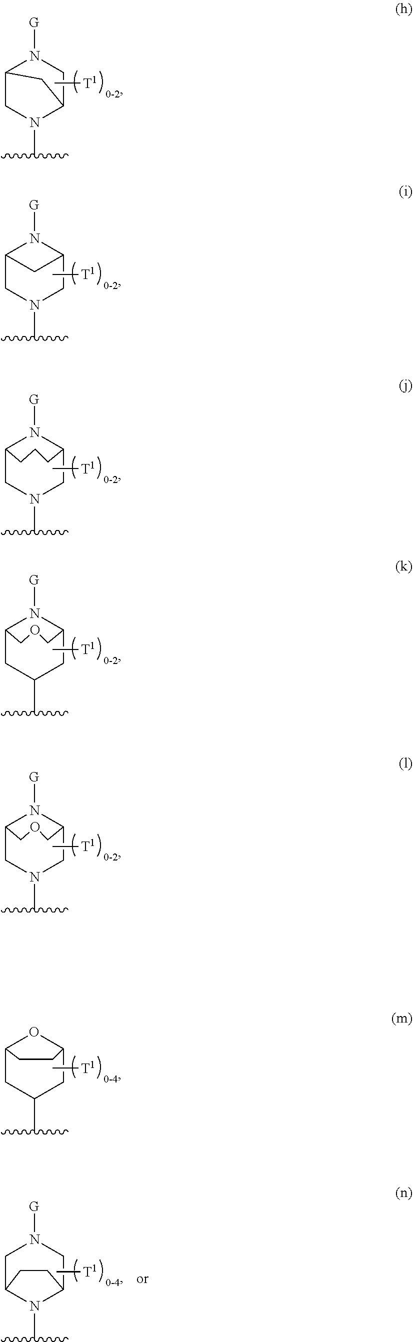

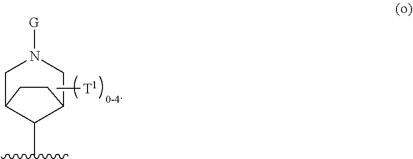

73. The compound according to claim 33, selected from Table IA, or a pharmaceutically acceptable salt thereof:
Lengthy table referenced here
US20230081426A1-20230316-T00001
Please refer to the end of the specification for access instructions.

74. A pharmaceutical composition comprising a compound according to claim 33 and a pharmaceutically acceptable carrier.

75. The pharmaceutical composition according to claim 74, further comprising a second pharmaceutical agent.

76. A method for treating a subject with a disease or condition mediated by KRAS, comprising administering to the subject an effective amount of a compound in claim 33, or a pharmaceutically acceptable salt, a solvate, a tautomer, a stereoisomer, or a deuterated analog thereof.

77. The method of claim 76, wherein the disease or condition is a neoplastic disorder, a cancer, an age-related disease, an inflammatory disorder, a cognitive disorder or a neurodegenerative disease.

78. The method of claim 77, wherein the disease or condition is pancreatic cancer, colorectal cancer, malignant solid tumor, rectal carcinoma, endometrial endometriod carcinoma, appendix carcinoma, mucinous adenocarcinoma, cholangiocarcinoma, extrahepatic cholangiocarcinoma, bladder cancer, bladder urothelial carcinoma, lung cancer, ampulla of vater carcinoma, ovarian cancer, gastric adenocarcinoma, malignant small intestinal neoplasm, poorly differentiated adenocarcinoma, acute myeloid leukemia, breast cancer, breast invasive ductal carcinoma, ampulla vater pancreatobiliary type adenocarcinoma, glioma, multiple myeloma, myelodysplastic syndrome, or small intestine carcinoma.

79. The method of claim 78, wherein the disease or condition is pancreatic cancer, colorectal cancer, malignant solid tumor, rectal carcinoma, lung cancer, acute myeloid leukemia, glioma, multiple myeloma, or myelodysplastic syndrome.

80. The method of claim 79, wherein the disease or condition is pancreatic carcinoma, pancreatic ductal adenocarcinoma, pancreatic adenocarcinoma, colorectal carcinoma, colorectal adenocarcinoma, malignant colorectal neoplasm, malignant solid tumor, rectal adenocarcinoma, lung adenocarcinoma, non-small cell lung cancer, small cell lung carcinoma, squamous cell lung carcinoma, acute myeloid leukemia, glioma, multiple myeloma, or myelodysplastic syndrome.

81. The method of claim 77, wherein the disease or condition is mediated by KRAS G12D.

82. The method of claim 81, wherein the disease or condition is pancreatic ductal adenocarcinoma colorectal carcinoma, pancreatic carcinoma, pancreatic adenocarcinoma, malignant colorectal neoplasm patients, malignant colorectal neoplasm, rectal carcinoma, lung adenocarcinoma, non-small cell lung carcinoma, colorectal carcinoma, malignant solid tumor, acute myeloid leukemia, squamous cell lung carcinoma, small cell lung carcinoma, glioma, myelodysplastic syndrome, breast cancer, gastric carcinoma, ovarian carcinoma, multiple myeloma patients, hepatocellular carcinoma, head and neck squamous cell carcinoma, glioblastoma or thyroid gland undifferentiated carcinoma.

83. The method of claim 77, wherein the disease or condition is mediated by KRAS G12V.

84. The method of claim 83, wherein the disease or condition is pancreatic ductal adenocarcinoma, pancreatic ductal adenocarcinoma, colorectal adenocarcinoma patients, colorectal carcinoma, rectal adenocarcinoma, pancreatic carcinoma, pancreatic adenocarcinoma, malignant colorectal neoplasm, lung adenocarcinoma, non-small cell lung carcinoma, colorectal carcinoma, malignant solid tumor, acute myeloid leukemia, squamous cell lung carcinoma, small cell lung carcinoma, glioma, myelodysplastic syndrome or multiple myeloma.

85. The method of claim 76, further comprising administering one or more additional therapeutic agents.

86. The method of claim 85, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from the group consisting of azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immunotherapy agent selected from a PD-1 or PD-L1 inhibitor; v) a hormone or hormone antagonist selected from the group consisting of enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38 (7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, AEE-788, AG-013736, AMG 706, AMN107, BMS-354825, BMS-599626, UCN-01 (7-hydroxystaurosporine), vemurafenib, dabrafenib, trametinib, cobimetinib selumetinib and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; and xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, a mTOR inhibitor, a PI3K inhibitor, a Cdk4 inhibitor, an Akt inhibitor, a Hsp90 inhibitor, a famesyltransferase inhibitor or an aromatase inhibitor (anastrozole letrozole exemestane); xvi) a Mek inhibitor; xvii) a tyrosine kinase inhibitor; xviii) a c-Kit mutant inhibitor, xix) an EGFR inhibitor, a PD-1 inhibitor, or xx) an epigenetic modulator.
